(19) United States
(12) Patent Application Publication
(10) Pub. No.: US 2026/0306650 A1
(43) Pub. Date: Oct. 8, 2026

(54) CAMPTOTHECIN DRUG CONJUGATE AND USE THEREOF

(71) Applicant: Shanghai Zaimo Biotechnology Co., Ltd., Shanghai (CN)

(72) Inventor: Dapeng SUN, Shanghai (CN)

(21) Appl. No.: 19/574,817

(22) Filed: Mar. 23, 2026

Related U.S. Application Data
(60) Provisional application No. 63/776,923, filed on Mar. 24, 2025.
(60) Provisional application No. 63/963,584, filed on Jan. 20, 2026.

(51) Int. Cl. A61K 47/68; A61P 35/00

(52) U.S. Cl. CPC A61K 47/68037; A61K 47/6851; A61K 47/6889; A61P 35/00

(57) ABSTRACT

Disclosed are a camptothecin drug conjugate and a use thereof. The present disclosure provides a drug conjugate of formula II or a pharmaceutically acceptable salt thereof. The camptothecin drug conjugate provided by the present disclosure has a good inhibitory effect on ovarian cancer, breast ductal carcinoma, breast cancer, pancreatic adenocarcinoma, pancreatic cancer or gastric cancer.

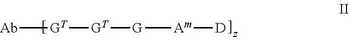

### CROSS-REFERENCE TO RELATED APPLICATIONS

The present application claims the benefit of priority to U.S. Patent Application No. 63/776,923 filed on Mar. 24, 2025 and U.S. Patent Application No. 63/963,584 filed on Jan. 20, 2026, the contents of which are incorporated herein by reference in their entireties.

### REFERENCE TO SEQUENCE LISTING

The instant application contains a Sequence Listing as an XML file entitled “BSP26431887US-SEQ” created on Mar. 20, 2026, and having a size of 26,784 bytes. The Sequence Listing is incorporated by reference herein.

### TECHNICAL FIELD

Provided herein are camptothecin, camptothecin-based linker-payload, drug conjugates and protein-drug conjugates comprising silicon containing functionalities, and methods and uses for treating diseases comprising administrating the protein-drug conjugates comprising silicon containing functionalities.

### BACKGROUND

Antibody-drug conjugates (ADCs) are proteins that are covalently tethered to a biologically active small molecule, also known as a payload. The antibody in ADCs provided a methodology to deliver the biologically active payload to tissues with antibody specific antigens. This delivery strategy potentially expands the therapeutic index (TI) for high potency payload in clinical applications. Up to now, around 13 ADCs have been approved by FDA.

Camptothecins are a class of natural products found in 1966 to target the topoisomerase I, which is crucial to DNA transcription and replication. It is originally isolated from the bark and stem of Camptotheca acuminata, which is a native tree in China. Among all camptothecins, irinotecan, topotecan and belotecan are approved by the regulatory institutes for the treatment of colorectal and ovarian cancer.

### SUMMARY

The technical problem to be solved by the present disclosure is that the type of camptothecin antibody-drug conjugate in the prior art is relatively single, and therefore, the present disclosure provides a camptothecin drug conjugate and a use thereof. The camptothecin drug conjugate provided by the present disclosure has a good inhibitory effect on ovarian cancer, breast ductal carcinoma, breast cancer, pancreatic adenocarcinoma, pancreatic cancer or gastric cancer; especially showing good inhibitory effects on ovarian cancer and HER2-positive drug-resistant breast cancer.

The present disclosure adopts the following technical solutions to solve the above technical problem.

The present disclosure provides a drug conjugate of formula II or a pharmaceutically acceptable salt thereof,

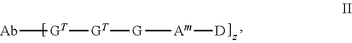

wherein
Ab is an antibody or an antigen-binding fragment thereof,
G^{T} is a group connected to an antibody or an antigen-binding fragment thereof,
G^{L} is a single bond or a linking group;

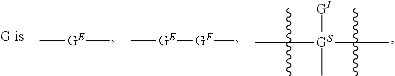

wherein G^{E} is connected to G^{L};
each G^{E} is independently a polypeptide residue consisting of

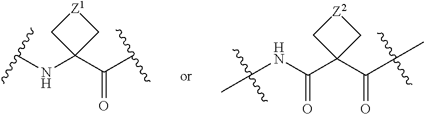

and t amino acid residues, each of the amino acid residues is independently a natural amino acid residue or a non-natural amino acid residue, and t is an integer of 1-10;
Z¹ and Z² are independently —CH₂— or O;
G^{F} and G^{S} are a self-immolative group;
G^{I} is

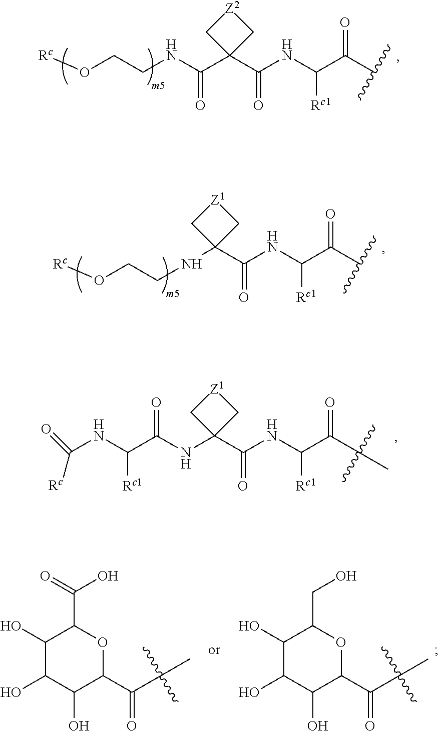

each R^{c} is independently C₁₋₆ alkyl;
each R^{c1} is independently

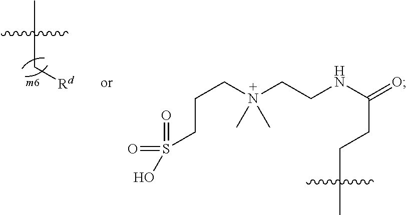

each R^{d} is independently carboxyl,

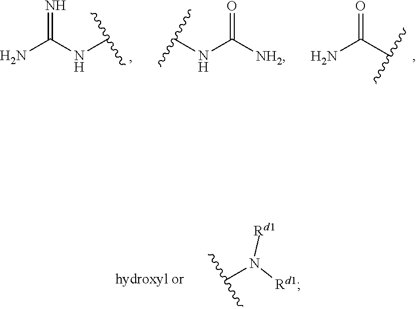

each R^{d1} is independently H or C₁₋₆ alkyl;
m5 and m6 are each independently 1, 2, 3, 4, 5 or 6;
A^{m} is a single bond, a natural amino acid residue or a non-natural amino acid residue;
D is

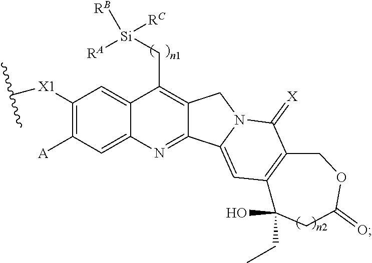

X1 is NH or O;
A is H, halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl;
R^{A}, R^{B} and R^{C} are independently H, halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl;
or, any two of R^{A}, R^{B} and R^{C} together with the silicon atom to which they are attached form a C₅₋₆ cycloalkyl ring or a 5- to 6-membered heterocyclyl ring, and the 5- to 6-membered heterocyclyl optionally further comprises one, two or three types of heteroatoms selected from N, O and S, and the number of the heteroatoms in the 5- to 6-membered heterocyclyl is independently 1, 2 or 3;
X is O or S;
n1 and n2 are independently 0, 1, 2 or 3;
z is an integer or decimal of 1-16; (z is the ratio of drug D to the antibody or the antigen-binding fragment thereof);
5- to 10-membered heteroaryl and 3- to 6-membered heterocyclyl each comprise one, two or three types of heteroatoms selected from N, O and S, and the number of the heteroatoms is independently 1, 2 or 3.

In a certain embodiment, in the drug conjugate of formula II, some groups have the following definitions, and the definitions of groups not mentioned are as described in any one of the embodiments of the present disclosure (the content of this paragraph is hereinafter referred to as “in a certain embodiment”).

In a certain embodiment, Ab is an antibody.

In a certain embodiment, the antibody is an antibody selected from one or more of anti-Her2 antibodies, anti-folate receptor α antibodies, anti-MUC1 antibodies and anti-human IgG1 antibodies, for example, an antibody selected from one or two of anti-Her2 antibodies and anti-MUC1 antibodies.

In a certain embodiment, the antibody is a monoclonal antibody or a bispecific antibody.

In a certain embodiment, the monoclonal antibody is an anti-Her2 antibody, an anti-folate receptor α antibody, an anti-MUC1 antibody or an anti-human IgG1 antibody.

In a certain embodiment, the bispecific antibody is an anti-Her2 and MUC1 antibody.

In a certain embodiment, the anti-Her2 antibody is a Trastuzumab antibody or a variant thereof, an FG1-Tsc antibody or a variant thereof, an FG1-Tsc thiomab antibody or a variant thereof, a Gati-Tsc antibody or a variant thereof, or a 186D-Tsc antibody or a variant thereof.

In a certain embodiment, the variant of the Trastuzumab antibody has at least 90%, at least 95%, at least 96%, at least 97%, at least 98%, at least 99%, at least 99.5% or at least 99.9% homology to the sequence of the Trastuzumab antibody; preferably, the variant of the Trastuzumab antibody has the same CDR sequences as the Trastuzumab antibody.

In a certain embodiment, the heavy chain of the FG1-Tsc antibody has an amino acid sequence as shown in SEQ ID NO: 12, and the light chain of the FG1-Tsc antibody has an amino acid sequence as shown in SEQ ID NO: 13; the variant of the FG1-Tsc antibody has at least 90%, at least 95%, at least 96%, at least 97%, at least 98%, at least 99%, at least 99.5% or at least 99.9% homology to the sequence of the FG1-Tsc antibody; preferably, the variant of the FG1-Tsc antibody has the same CDR sequences as the FG1-Tsc antibody.

In a certain embodiment, the heavy chain of the FG1-Tsc thiomab antibody has an amino acid sequence as shown in SEQ ID NO: 20, and the light chain of the FG1-Tsc thiomab antibody has an amino acid sequence as shown in SEQ ID NO: 21; the variant of the FG1-Tsc thiomab antibody has at least 90%, at least 95%, at least 96%, at least 97%, at least 98%, at least 99%, at least 99.5% or at least 99.9% homology to the sequence of the FG1-Tsc thiomab antibody; preferably, the variant of the FG1-Tsc thiomab antibody has the same CDR sequences as the FG1-Tsc thiomab antibody.

In a certain embodiment, the heavy chain of the Gati-Tsc antibody has an amino acid sequence as shown in SEQ ID NO: 14, and the light chain of the Gati-Tsc antibody has an amino acid sequence as shown in SEQ ID NO: 15; the variant of the Gati-Tsc antibody has at least 90%, at least 95%, at least 96%, at least 97%, at least 98%, at least 99%, at least 99.5% or at least 99.9% homology to the sequence of the Gati-Tsc antibody; preferably, the variant of the Gati-Tsc antibody has the same CDR sequences as the Gati-Tsc antibody.

In a certain embodiment, the heavy chain of the 186D-Tsc antibody has an amino acid sequence as shown in SEQ ID NO: 16, and the light chain of the 186D-Tsc antibody has an amino acid sequence as shown in SEQ ID NO: 17; the variant of the 186D-Tsc antibody has at least 90%, at least 95%, at least 96%, at least 97%, at least 98%, at least 99%, at least 99.5% or at least 99.9% homology to the sequence of the 186D-Tsc antibody; preferably, the variant of the 186D-Tsc antibody has the same CDR sequences as the 186D-Tsc antibody.

In a certain embodiment, the anti-folate receptor α antibody is a v30384 antibody or a variant thereof, the heavy chain of the v30384 antibody has an amino acid sequence as shown in SEQ ID NO: 3 and an amino acid sequence as shown in SEQ ID NO: 4, and the light chain of the v30384 antibody has an amino acid sequence as shown in SEQ ID NO: 5; the variant of the v30384 antibody has at least 90%, at least 95%, at least 96%, at least 97%, at least 98%, at least 99%, at least 99.5% or at least 99.9% homology to the sequence of the v30384 antibody; preferably, the variant of the v30384 antibody has the same CDR sequences as the v30384 antibody.

In a certain embodiment, the anti-MUC1 antibody is an FG1 antibody or a variant thereof, a Gatipotuzumab antibody or a variant thereof, a 186D antibody or a variant thereof, an FG1-Tsc antibody or a variant thereof, a FG1-Tsc thiomab antibody or a variant thereof, a Gati-Tsc or a variant thereof, or a 186D-Tsc antibody or a variant thereof,

In a certain embodiment, the heavy chain of the FG1 antibody has an amino acid sequence as shown in SEQ ID NO: 6, and the light chain of the FG1 antibody has an amino acid sequence as shown in SEQ ID NO: 7; the variant of the FG1 antibody has at least 90%, at least 95%, at least 96%, at least 97%, at least 98%, at least 99%, at least 99.5% or at least 99.9% homology to the sequence of the FG1 antibody; preferably, the variant of the FG1 antibody has the same CDR sequences as the FG1 antibody.

In a certain embodiment, the heavy chain of the Gatipotuzumab antibody has an amino acid sequence as shown in SEQ ID NO: 8, and the light chain of the Gatipotuzumab antibody has an amino acid sequence as shown in SEQ ID NO: 9; the variant of the Gatipotuzumab antibody has at least 90%, at least 95%, at least 96%, at least 97%, at least 98%, at least 99%, at least 99.5% or at least 99.9% homology to the sequence of the Gatipotuzumab antibody; preferably, the variant of the Gatipotuzumab antibody has the same CDR sequences as the Gatipotuzumab antibody.

In a certain embodiment, the heavy chain of the 186D antibody has an amino acid sequence as shown in SEQ ID NO: 10, and the light chain of the 186D antibody has an amino acid sequence as shown in SEQ ID NO: 11; the variant of the 186D antibody has at least 90%, at least 95%, at least 96%, at least 97%, at least 98%, at least 99%, at least 99.5% or at least 99.9% homology to the sequence of the 186D antibody; preferably, the variant of the 186D antibody has the same CDR sequences as the 186D antibody.

In a certain embodiment, the anti-human IgG1 antibody is a DNP antibody (anti-deoxyribonucleoprotein antibody), the heavy chain variable region of the anti-DNP antibody has an amino acid sequence as shown in SEQ ID NO: 18, and the light chain variable region of the anti-DNP antibody has an amino acid sequence as shown in SEQ ID NO: 19.

In a certain embodiment, the anti-Her2 and MUC1 antibody is the G1-Tsc antibody or a variant thereof, the FG1-Tsc thiomab antibody or a variant thereof, the Gati-Tsc antibody or a variant thereof, or the 186D-Tsc antibody or a variant thereof.

In a certain embodiment, the constant region of the antibody is derived from a human antibody heavy chain IgG1 constant region and a human antibody light chain kappa constant region.

In a certain embodiment, the antibody is a Trastuzumab antibody, a v30384 antibody, an FG1 antibody, a Gatipotuzumab antibody, a 186D antibody, an FG1-Tsc antibody, an FG1-Tsc thiomab antibody or a DNP antibody, for example, a Trastuzumab antibody, an FG1 antibody or an FG1-Tsc thiomab antibody, for another example, an FG1-Tsc thiomab antibody.

In a certain embodiment, the antibody is the variant of the Trastuzumab antibody, the variant of the v30384 antibody, the variant of the FG1 antibody, the variant of the Gatipotuzumab antibody, the variant of the 186D antibody, the variant of the FG1-Tsc antibody or the variant of the FG1-Tsc thiomab.

In a certain embodiment, G^{T} is

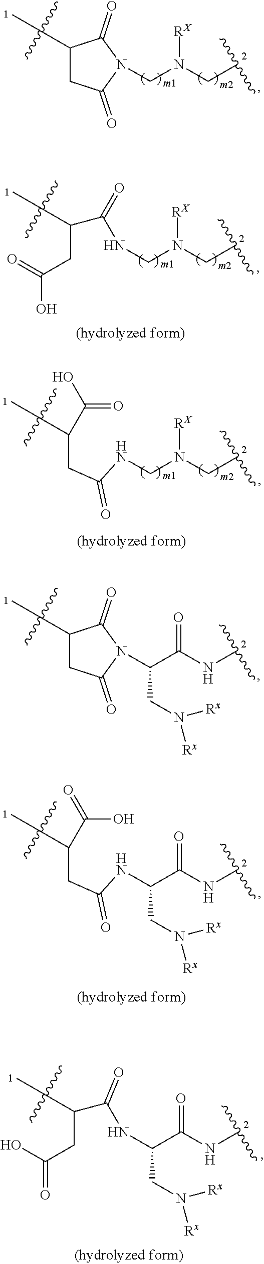

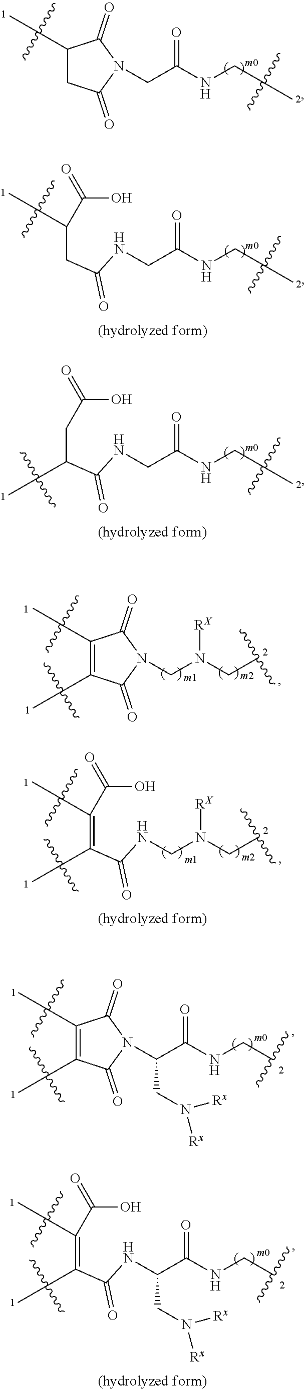

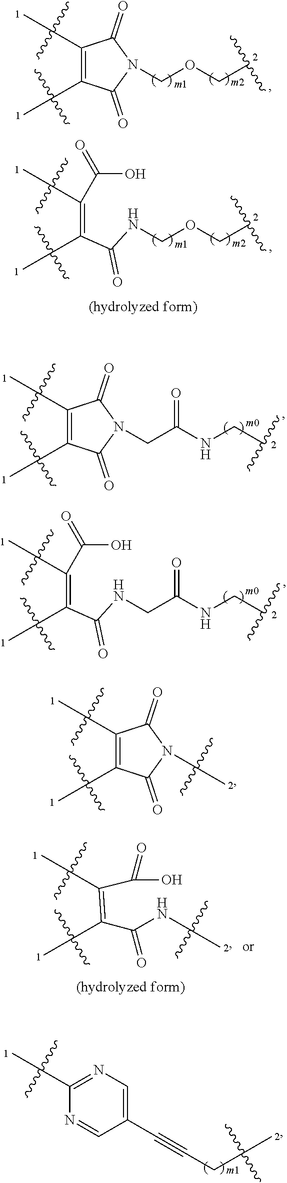

wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl.
preferably

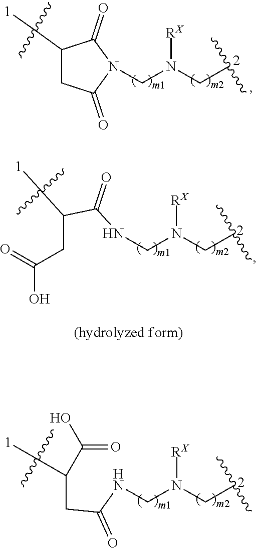

(hydrolyzed form) or

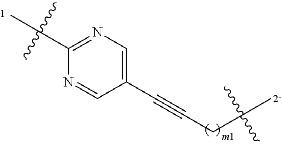 [SMILES: *CC#Cc1cnc(*)nc1]

In a certain embodiment, G^{T} is

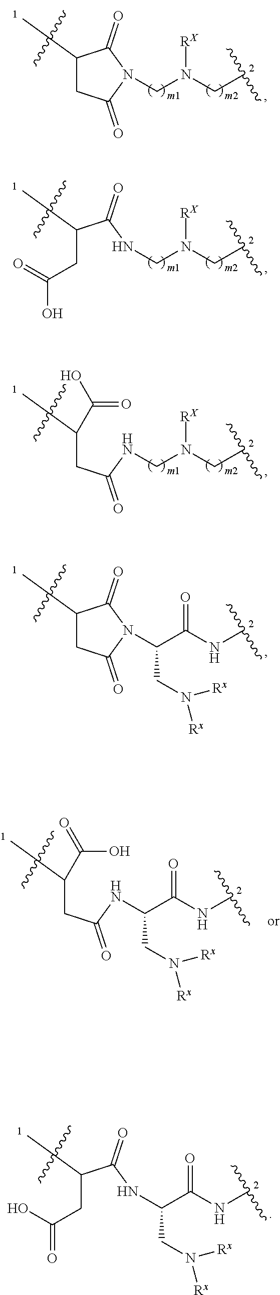

In a certain embodiment, G^{T} is

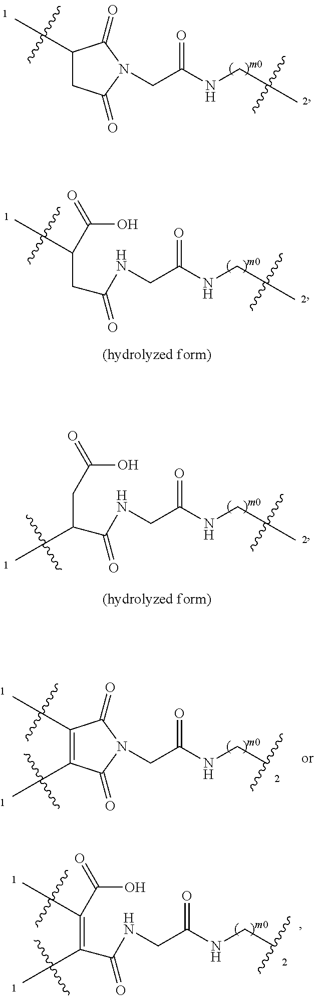

and m0 is 1, 2, 3, 4 or 5.

In a certain embodiment, each R^{x} is independently H, methyl, ethyl, n-propyl, isopropyl, n-butyl, isobutyl, sec-butyl or tert-butyl, for example, H or methyl.

In a certain embodiment, G^{T} is

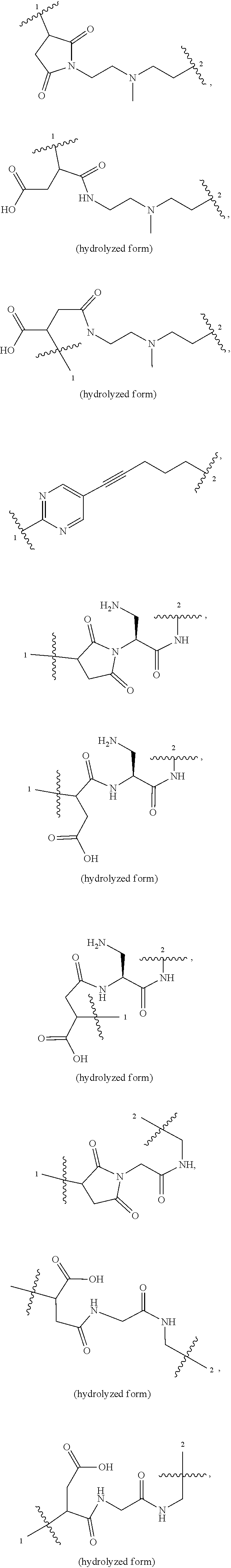

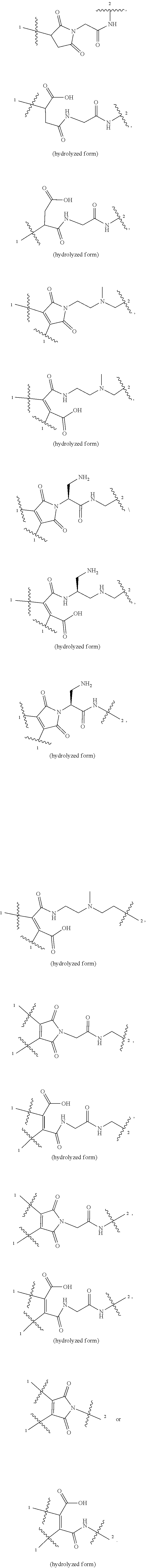

In a certain embodiment, G^{L} is -G^{L1}- or -G^{L1}-G^{L2}- and G^{L1} is connected to G^{T};
G^{L1} (G^{L}) is

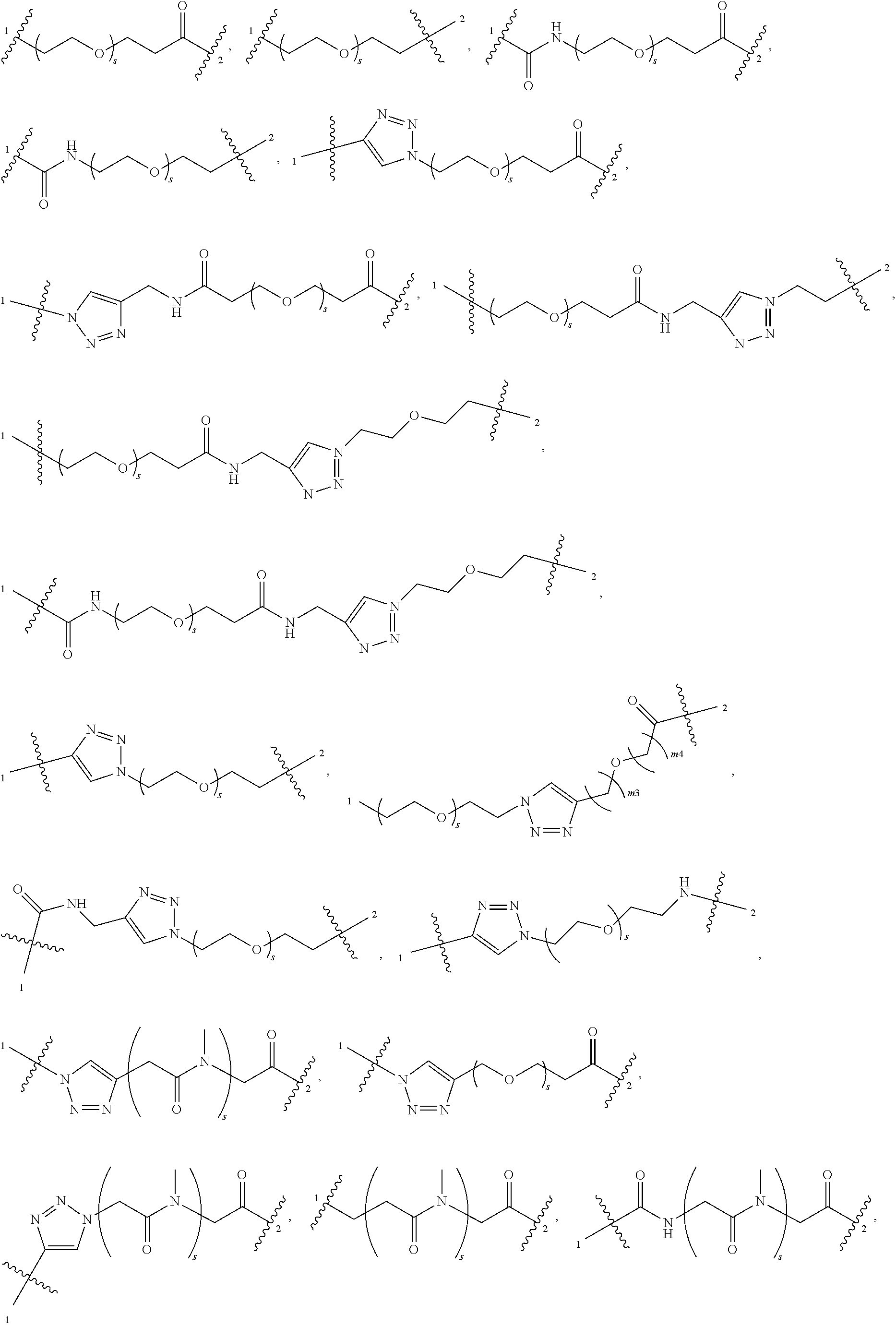

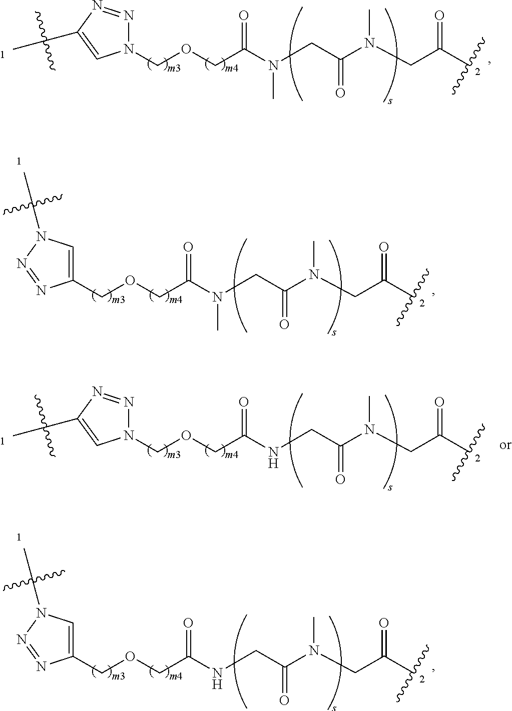

wherein position “1” is connected to G^{T}; position “2” is connected to G^{L2}, G^{E} or G^{S}.
m3 and m4 are each independently 1, 2, 3, 4 or 5;
each s is independently an integer from 0 to 30.

G^{L2} is a single amino acid residue or a short peptide composed of 2-6 amino acid residues; each amino acid residue is independently a natural amino acid or an unnatural amino acid residue.

In a certain embodiment, G^{L2} is

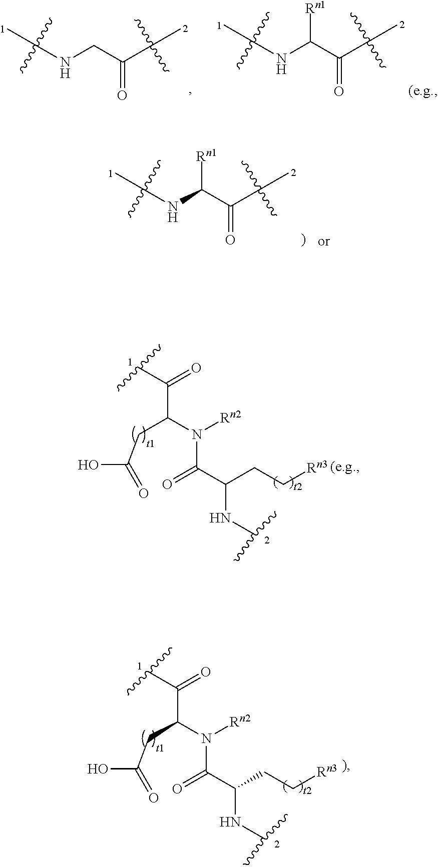

wherein position “1” is connected to G^{L1}; position “2” is connected to G^{E} or G^{S}; t1 and t2 are independently 1, 2, 3 or 4; Rⁿ¹ is C₁₋₄ alkyl, Rⁿ² is H or methyl, and Rⁿ³ is amino,

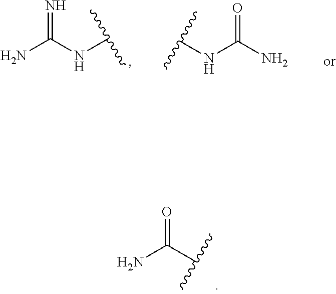

In a certain embodiment, G^{L2} is

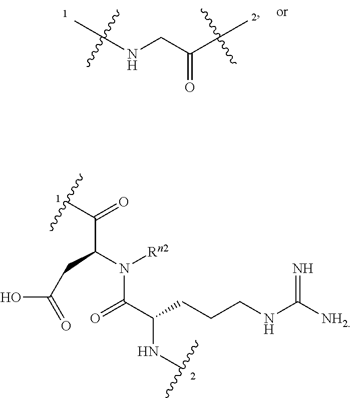

In a certain embodiment, G^{L1} is

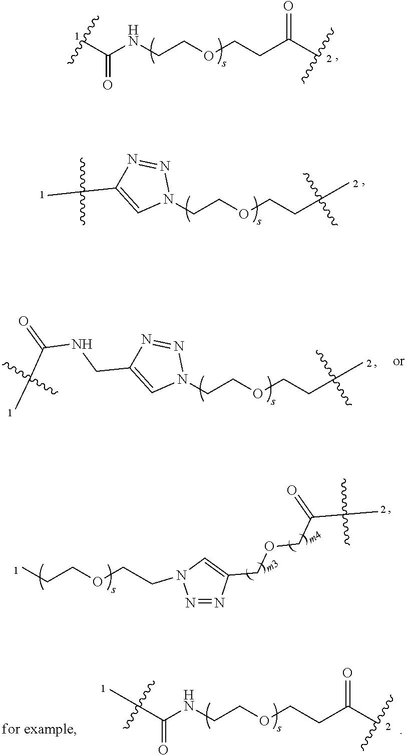

In a certain embodiment, G^{L1} is

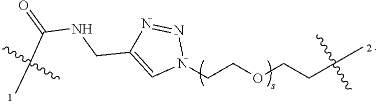

In a certain embodiment, G^{L1} is

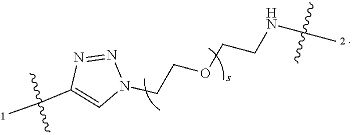

In a certain embodiment, G^{L1} is

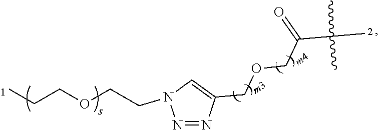

In a certain embodiment, each s is independently 1, 2, 3, 4, 5 or 6.

In a certain embodiment, G^{L1} (or G^{L}) is

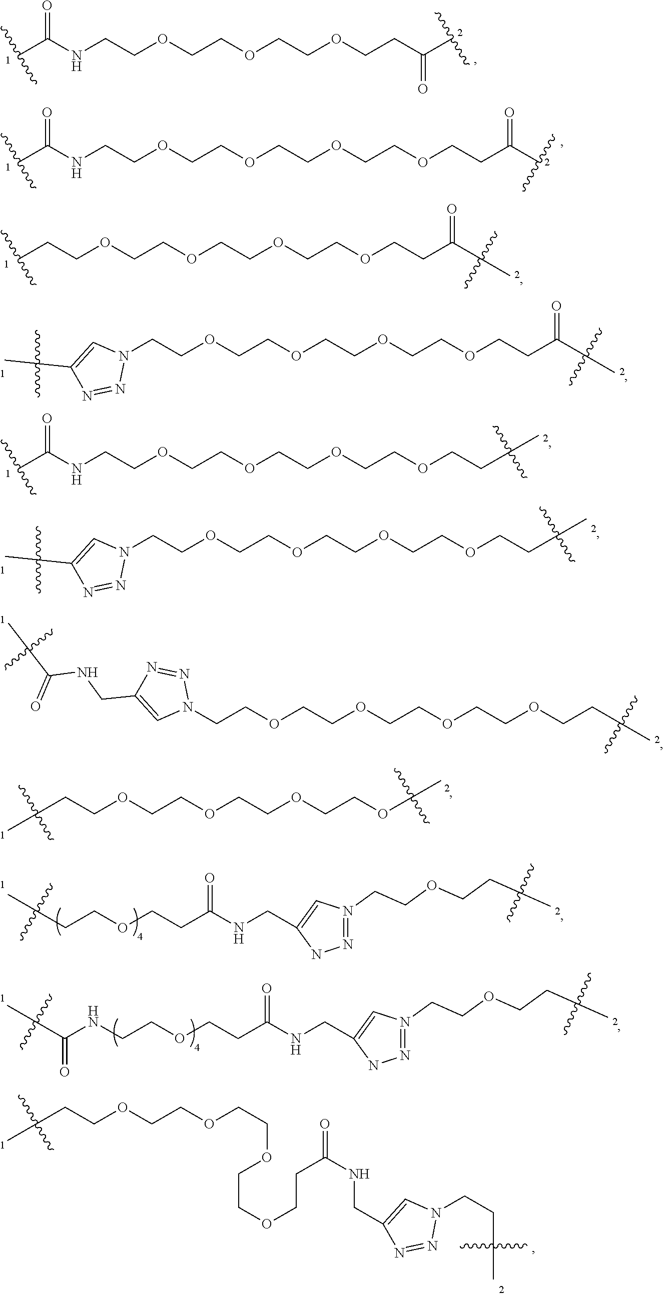

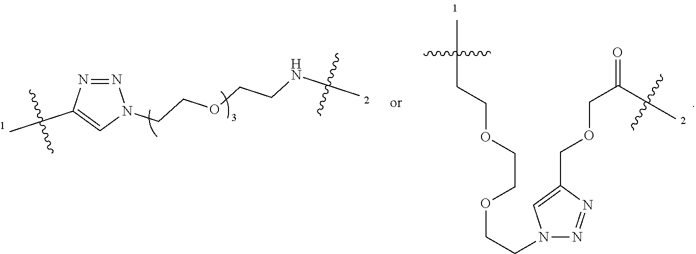

In a certain embodiment, -G^{T}-G^{L1}- is

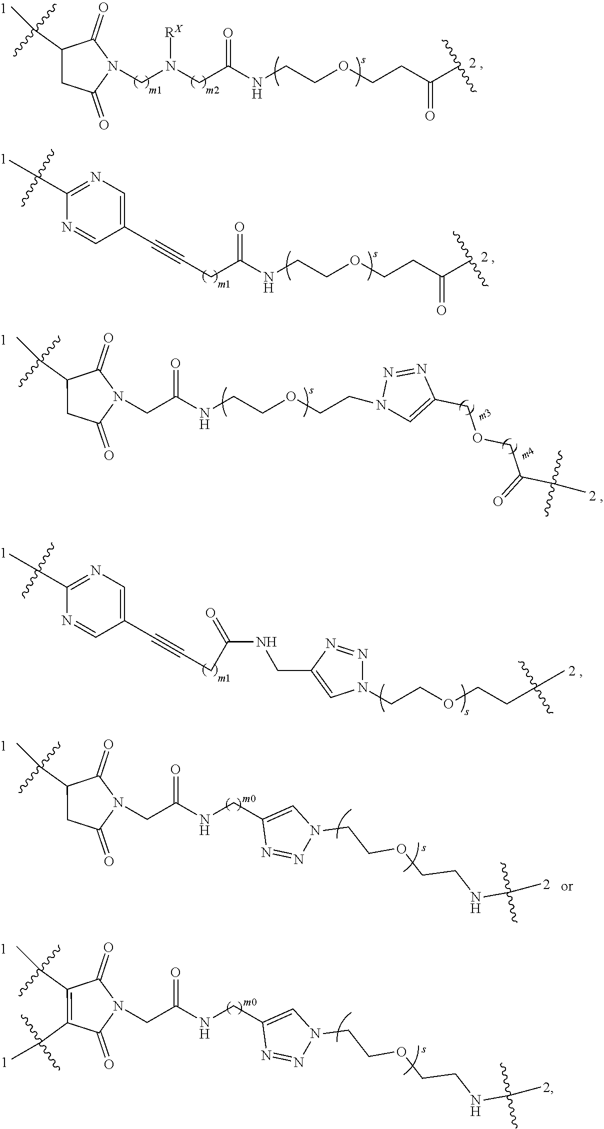

wherein position “1” is connected to the antibody, and position “2” is connected to G^{S} or G^{L2}-; preferably, -G^{T}-G^{L}- is -G^{T}-G^{L1}-.

In a certain embodiment, -G^{T}-G^{L1}- is

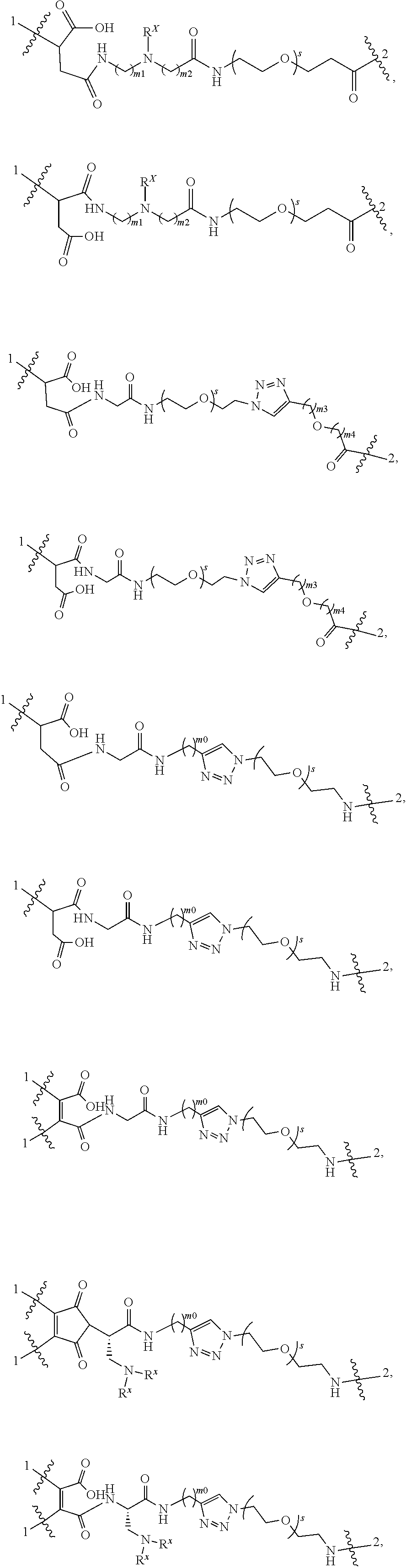

wherein position “1” is connected to the antibody, and position “2” is connected to G^{S} or G^{L2}-.

In a certain embodiment, G^{E} is

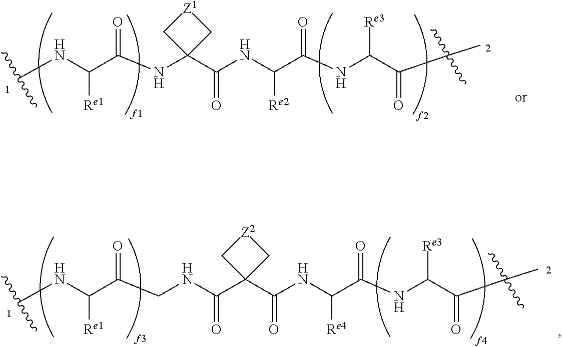

wherein position “1” is connected to G^{L}, and position “2” is connected to G^{F} or D;
Z¹ and Z² are independently —CH₂— or O;
R^{e1}, R^{e2}, R^{e3} and R^{e4} are independently H or —(CH₂)_{f5}—R^{ee},
R^{ee} is —OH, —COOH, —NH₂, —NR^{ee-1}R^{ee-2},

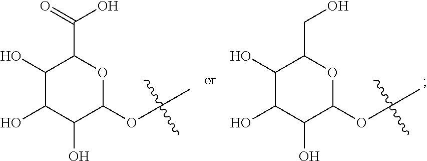 [SMILES: *OC1OC(C(=O)O)C(O)C(O)C1O ; *OC1OC(CO)C(O)C(O)C1O]

R^{ee-1} and R^{ee-2} are independently methyl, ethyl, n-propyl, isopropyl, n-butyl, isobutyl, sec-butyl or tert-butyl;
f1 and f2 are independently 1, 2, 3 or 4; f3 and f4 are independently 0, 1, 2, 3 or 4;
f5 is 1, 2, 3, 4, 5 or 6.
preferably, Z¹ and Z² are O.

In a certain embodiment, G^{E} is

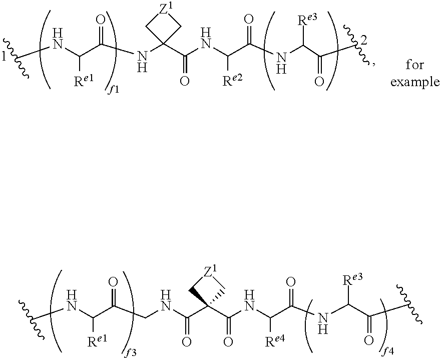

In a certain embodiment, G^{E} is

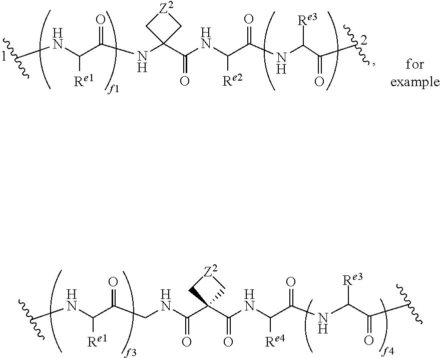

In a certain embodiment, G^{E} is

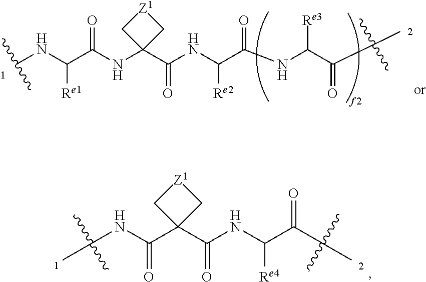

f2 is 1 or 2, for example,

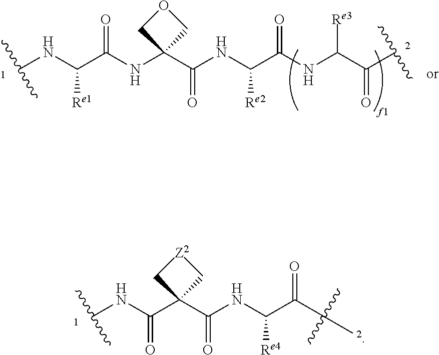

In a certain embodiment, each

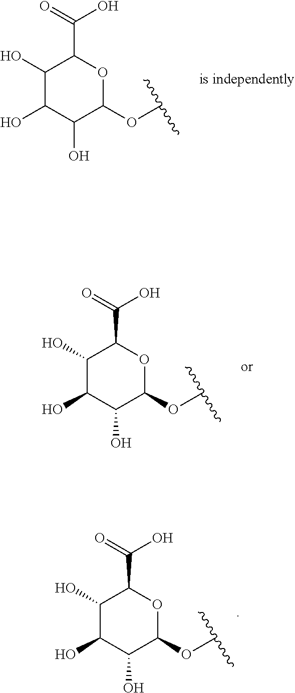 [SMILES: *OC1OC(C(=O)O)C(O)C(O)C1O ; *O[C@@H]1O[C@H](C(=O)O)[C@@H](O)[C@H](O)[C@H]1O ; *O[C@@H]1O[C@H](C(=O)O)[C@@H](O)[C@H](O)[C@H]1O]

In a certain embodiment, each

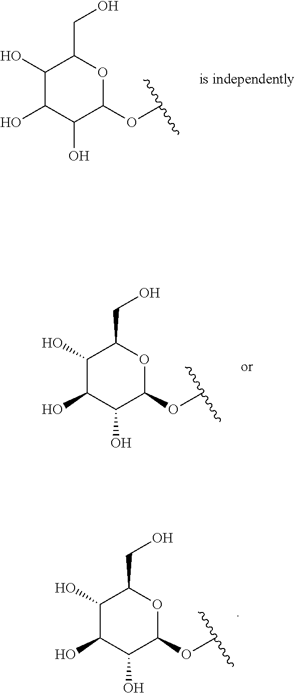 [SMILES: *OC1OC(CO)C(O)C(O)C1O ; *O[C@@H]1O[C@H](CO)[C@@H](O)[C@H](O)[C@H]1O ; *O[C@@H]1O[C@H](CO)[C@@H](O)[C@H](O)[C@H]1O]

In a certain embodiment, R^{e1} is —(CH₂)_{f5}—COOH, for example,

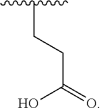 [SMILES: *CCC(=O)O]

In a certain embodiment, R^{e2} is —(CH₂)_{f5}—R^{ee}, R^{ee} is —OH, —NH₂, —NR^{ee-1}R^{ee-2} or

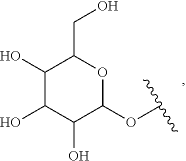 [SMILES: *OC1OC(CO)C(O)C(O)C1O]

for example, R^{e2} is

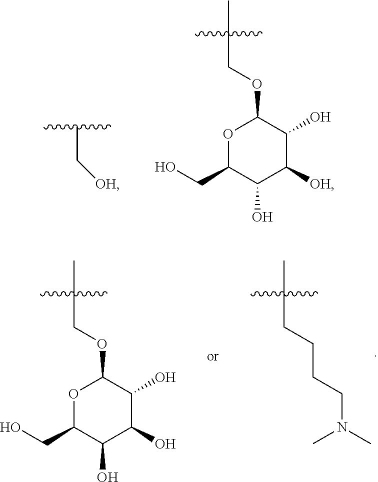

In a certain embodiment, R^{e3} is H.

In a certain embodiment, R^{e4} is —(CH₂)_{f5}—R^{ee}, R^{ee} is —OH, —NH₂ or —NR^{ee-1}R^{ee-2} for example,

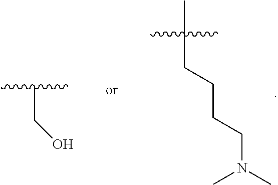

In a certain embodiment, G^{E} is

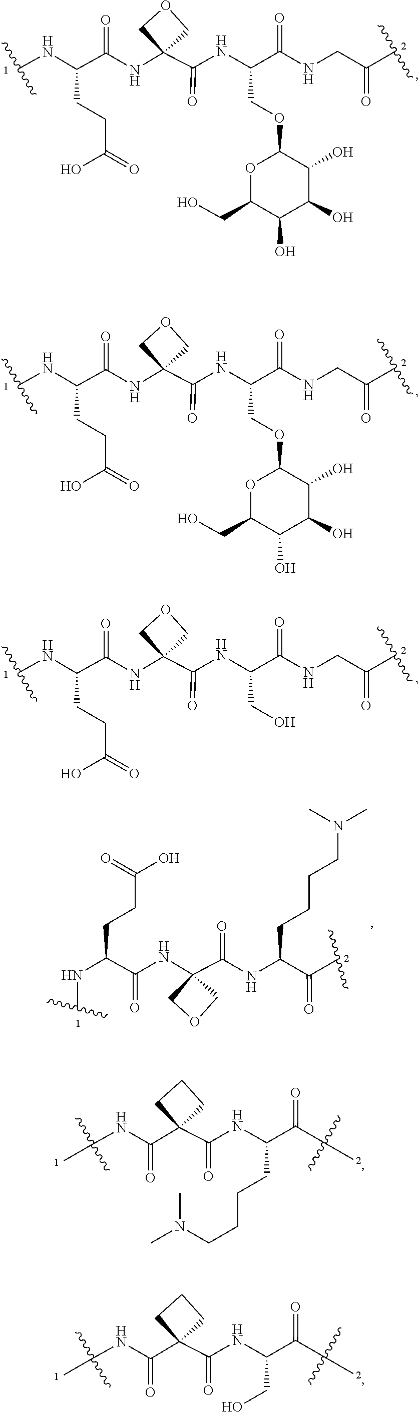

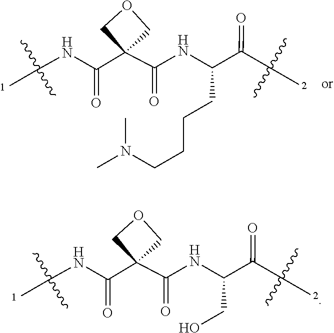

In a certain embodiment, G^{F} is

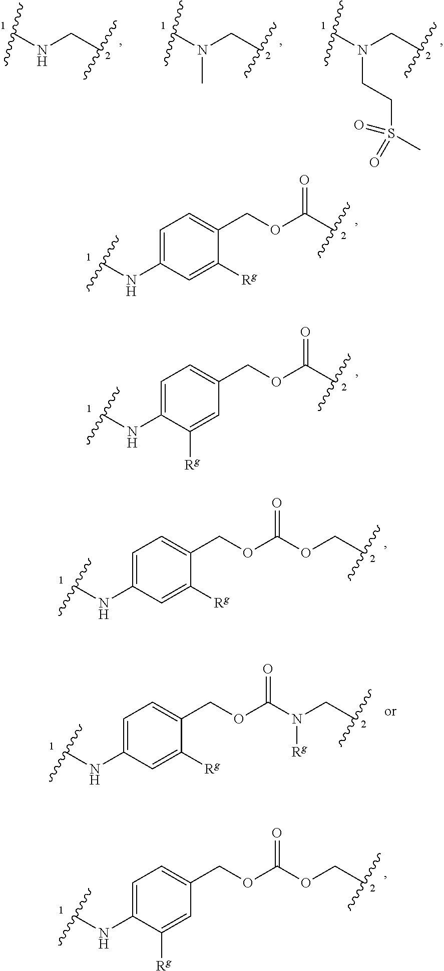

wherein position “1” is connected to G^{E}, and position “2” is connected to A^{m};
each R^{g} is independently H,

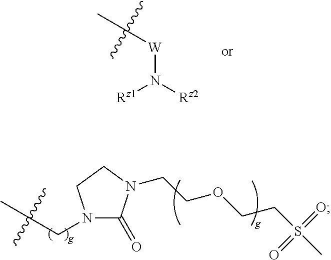

W is independently C₁₋₆ alkylene or C₂₋₆ alkynylene;
R^{z1} and R^{z2} are independently

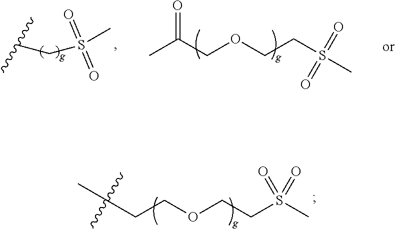

each g is independently 1, 2, 3, 4, 5 or 6.

In a certain embodiment, G^{F} is

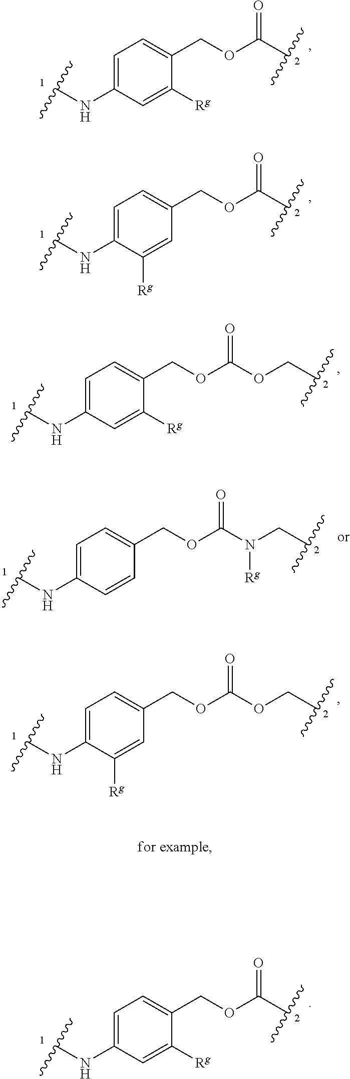

In a certain embodiment, G^{F} is

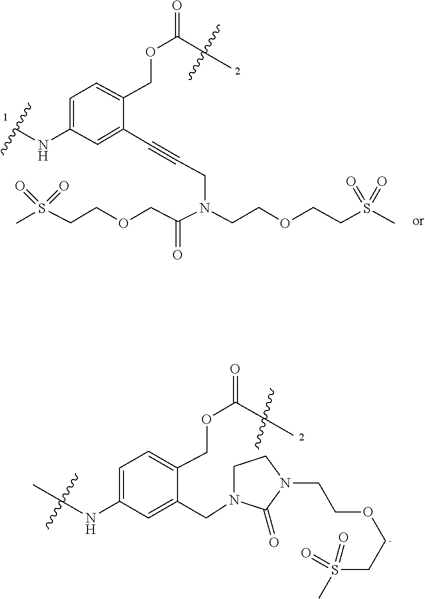 [SMILES: *Nc1ccc(COC(*)=O)c(C#CCN(CCOCCS(C)(=O)=O)C(=O)COCCS(C)(=O)=O)c1 ; *Nc1ccc(COC(*)=O)c(CN2CCN(CCOCCS(C)(=O)=O)C2=O)c1]

In a certain embodiment, G^{S} is

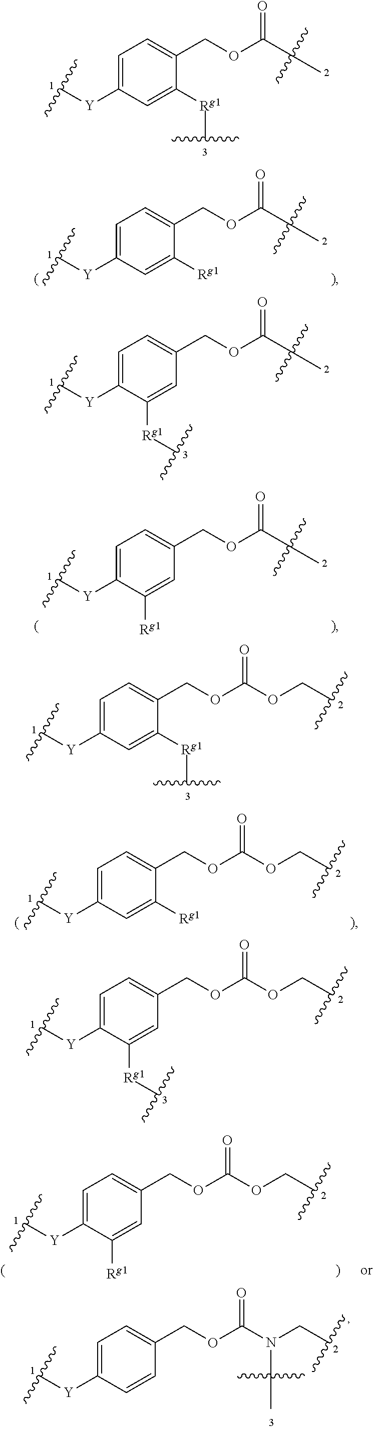

wherein each R^{g1} is independently

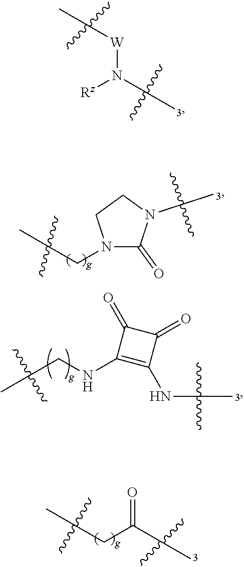

### or —NH—³;

wherein position “1” is connected to G¹, position “2” is connected to A^{m}, and position “3” is connected to G^{L};
each Y is independently NH or a single bond;
each W is independently C₁₋₆ alkylene or C₂₋₆ alkynylene;
each R^{z} is independently

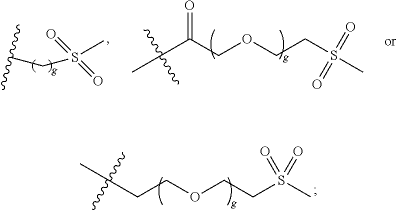

each g is independently 1, 2, 3, 4, 5 or 6.

In a certain embodiment, G^{S} is

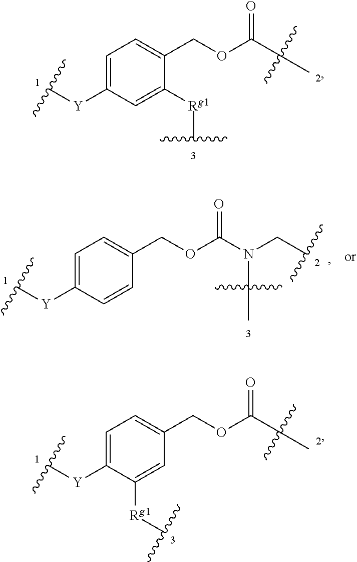

for example,

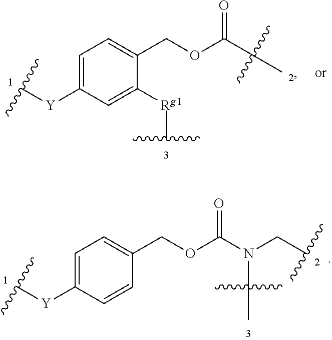

In a certain embodiment, each W is independently methylene,

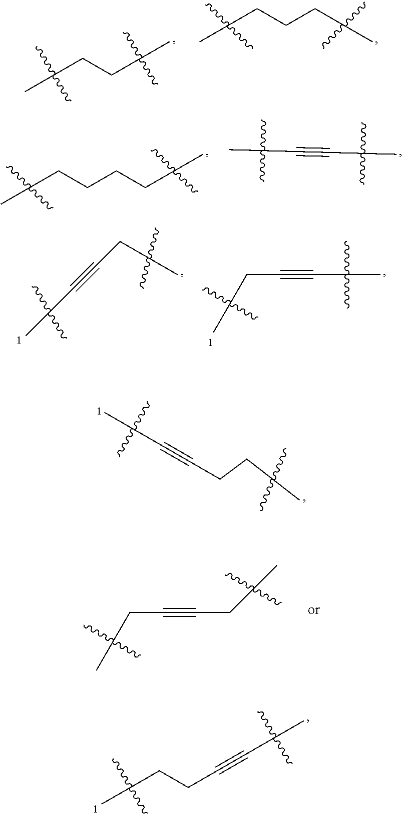

wherein position “1” is connected to a benzene ring; for example, each W is independently methylene or

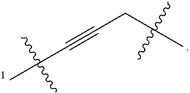 [SMILES: *C#CC*]

In a certain embodiment, R^{z} is

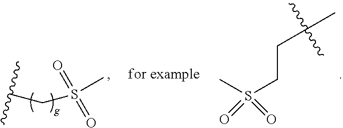

In a certain embodiment,

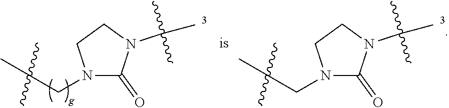

In a certain embodiment,

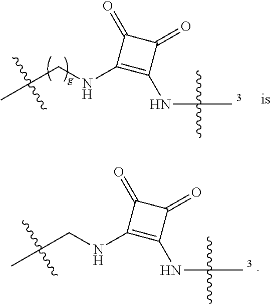

In a certain embodiment,

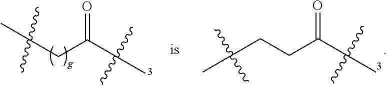

In a certain embodiment, G^{S} is

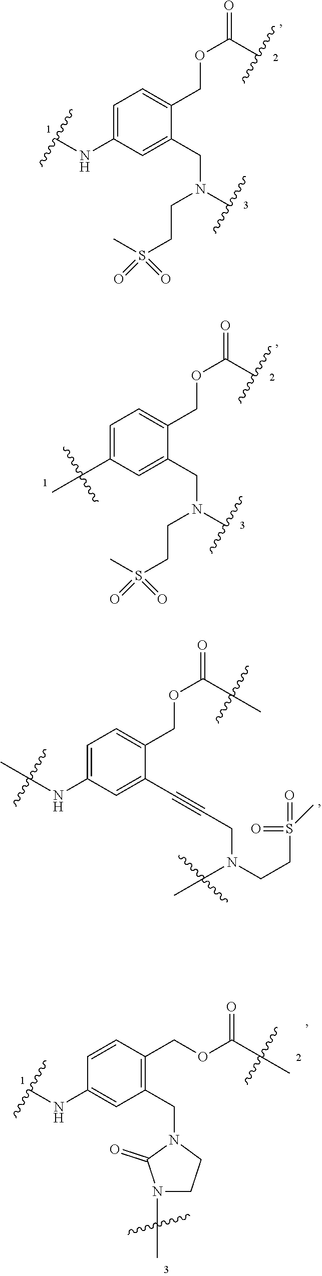 [SMILES: *Nc1ccc(COC(*)=O)c(CN(*)CCS(C)(=O)=O)c1 ; *C(=O)OCc1ccc(*)cc1CN(*)CCS(C)(=O)=O ; *Nc1ccc(COC(*)=O)c(C#CCN(*)CCS(C)(=O)=O)c1 ; *Nc1ccc(COC(*)=O)c(CN2CCN(*)C2=O)c1]

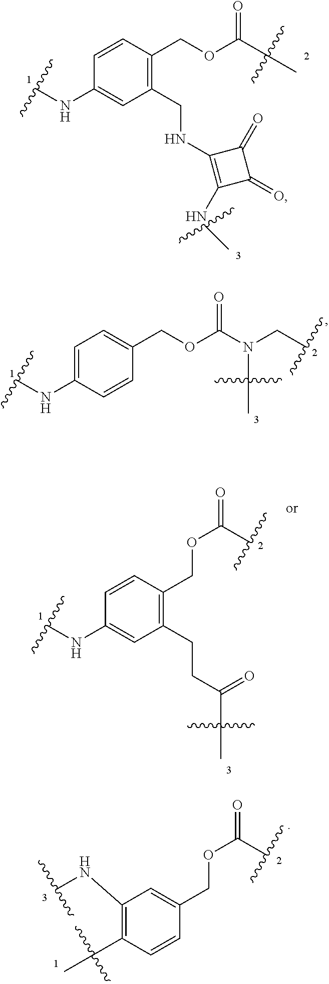

In a certain embodiment, G^{I} is

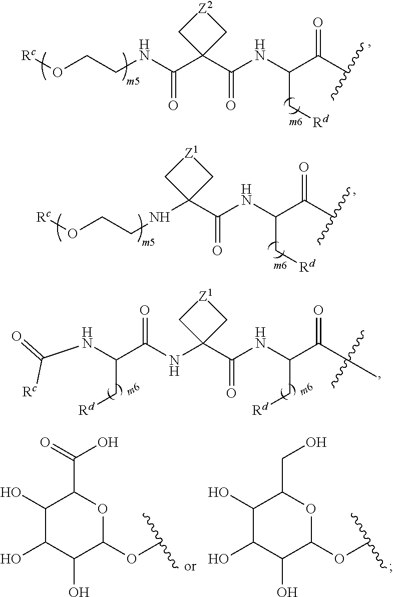

preferably, Z¹ and Z² are —CH₂—.

In a certain embodiment,

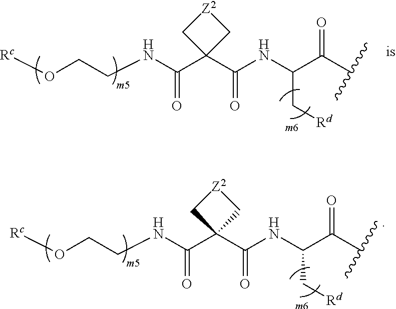

In a certain embodiment,

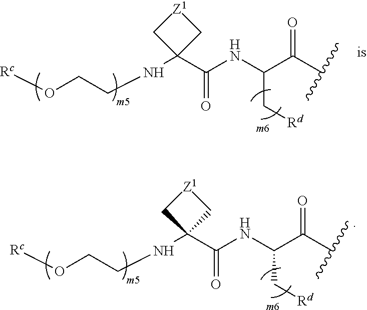

In a certain embodiment,

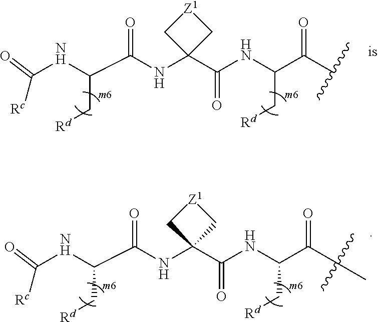

In a certain embodiment, G^{I} is

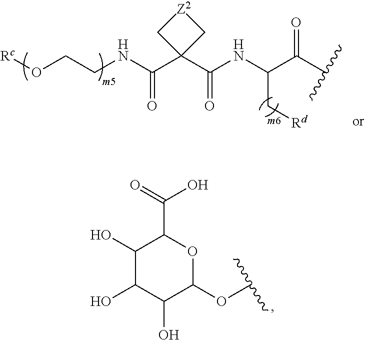

for example,

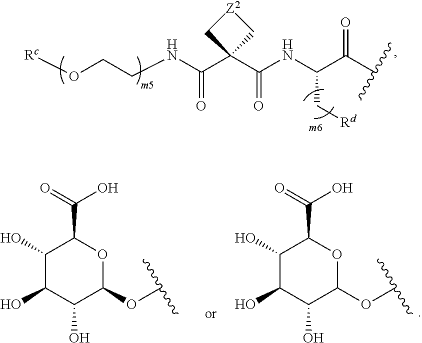

In a certain embodiment, G^{I} is

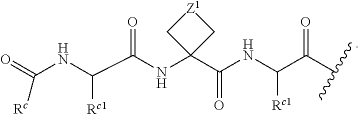

In a certain embodiment, R^{c} and R^{d1} are each independently methyl, ethyl, n-propyl, isopropyl, n-butyl, isobutyl, sec-butyl or tert-butyl, for example, methyl.

In a certain embodiment, G^{I} is

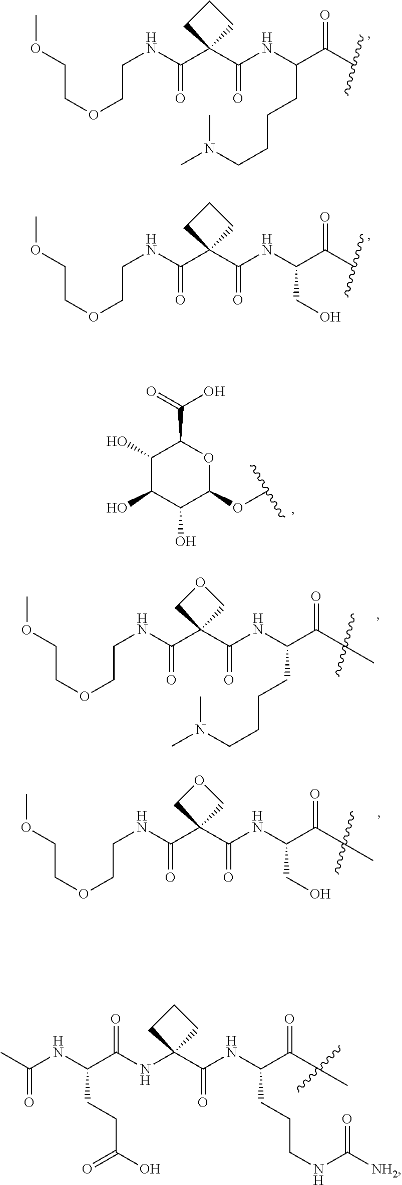

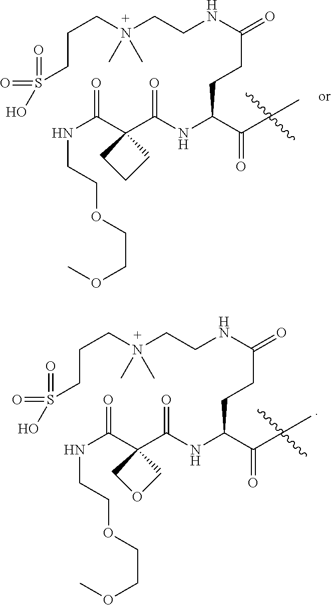

In a certain embodiment, A^{m} is a single bond or

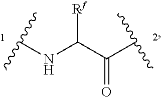

R^{f} is H, C₁₋₆ alkyl or —(CH₂)ₙ—R^{f1}, R^{f1} is —NH₂ or —NR^{ee-1}R^{ee-2}, n is 1, 2, 3, 4, 5 or 6, R^{ee-1} and R^{ee-2} are independently methyl, ethyl, n-propyl, isopropyl, n-butyl, isobutyl, sec-butyl or tert-butyl, wherein position “1” is connected to G^{E}, G^{F} or G^{S}, and position “2” is connected to D;
for example, A^{m} is a single bond,

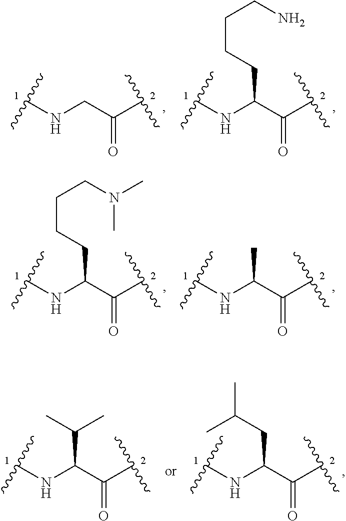

wherein position “1” is connected to G^{E}, G^{F} or G^{S}, and position “2” is connected to D; for another example, A^{m} is a single bond,

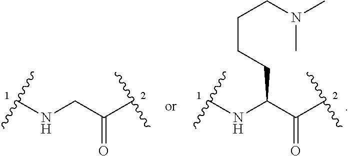 [SMILES: *NCC(*)=O ; *N[C@@H](CCCCN(C)C)C(*)=O]

In a certain embodiment, A is H, F, Cl, Br or I, for example, H or F.

In a certain embodiment, R^{A}, R^{B} and R^{C} are independently C₁₋₆ alkyl, for example, methyl, ethyl, n-propyl, isopropyl, n-butyl, isobutyl, sec-butyl or tert-butyl, for another example, methyl.

In a certain embodiment, X1 is N.

In a certain embodiment, X is O.

In a certain embodiment, n1 is 2.

In a certain embodiment, n2 is 0.

In a certain embodiment, D is

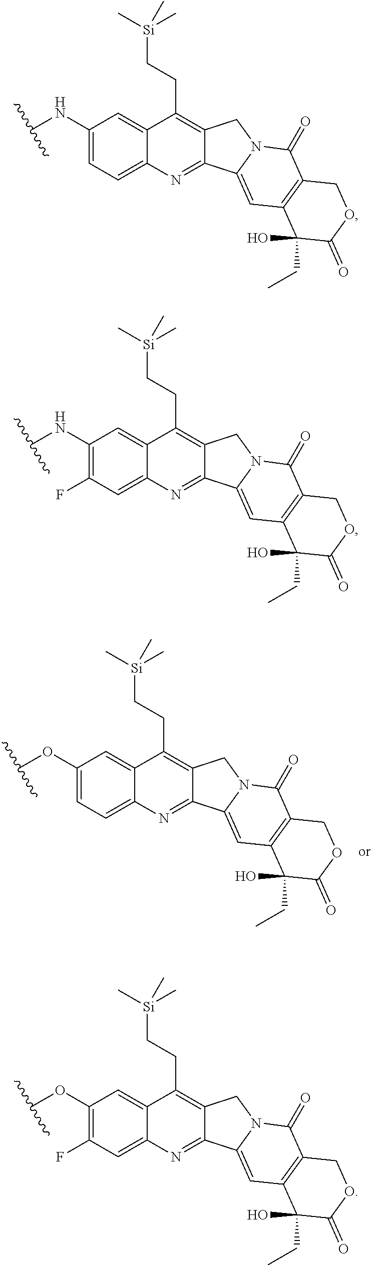 [SMILES: *Nc1ccc2nc3c(c(CC[Si](C)(C)C)c2c1)Cn1c-3cc2c(c1=O)COC(=O)[C@]2(O)CC ; *Nc1cc2c(CC[Si](C)(C)C)c3c(nc2cc1F)-c1cc2c(c(=O)n1C3)COC(=O)[C@]2(O)CC ; *Oc1ccc2nc3c(c(CC[Si](C)(C)C)c2c1)Cn1c-3cc2c(c1=O)COC(=O)[C@]2(O)CC ; *Oc1cc2c(CC[Si](C)(C)C)c3c(nc2cc1F)-c1cc2c(c(=O)n1C3)COC(=O)[C@]2(O)CC]

In a certain embodiment, z is an integer or decimal of 1-8, for example, an integer or decimal of 3-4, or an integer or decimal of 6-8.

In a certain embodiment, z is an integer or decimal of 3 to 5.

In a certain embodiment, z is 3.2, 3.3, 3.4, 3.5, 3.6, 3.7, 3.8, 3.9, 4, 4.1, 6.8, 6.9, 7, 7.1, 7.2, 7.4, 7.5, 7.3, 7.4, 7.6, 7.8, 7.9 or 8.0.

In a certain embodiment, the drug conjugate of formula II is a drug conjugate of formula IIa, IIb or IIc,

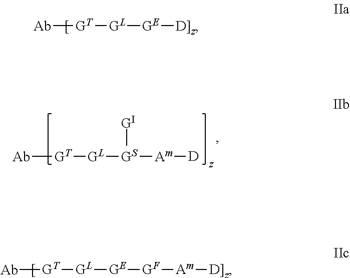

Ab, A^{m}, G^{T}, G^{L}, G^{E}, G^{F}, G^{S}, G^{I}, D and z are as described in any of the previous embodiments, wherein in IIa, G^{L} is G^{L1}.

In a certain embodiment, the drug conjugate of formula II is a drug conjugate of formula IIa,
Ab is an antibody selected from one or more of anti-Her2 antibodies, anti-folate receptor α antibodies, anti-MUC1 antibodies and anti-human IgG1 antibodies;
G^{T} is

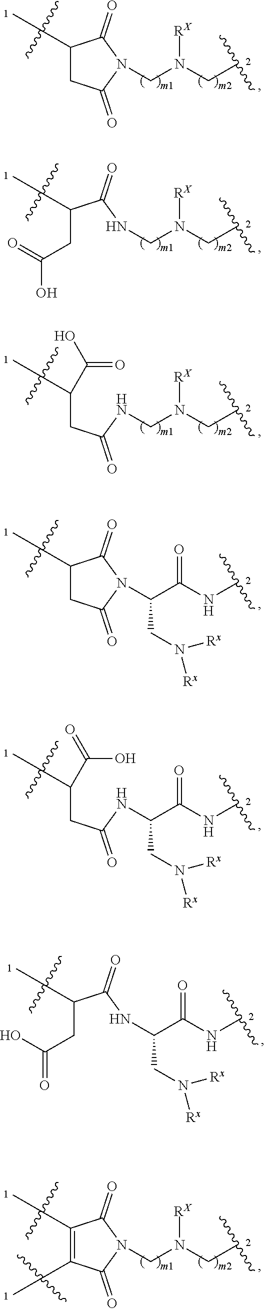

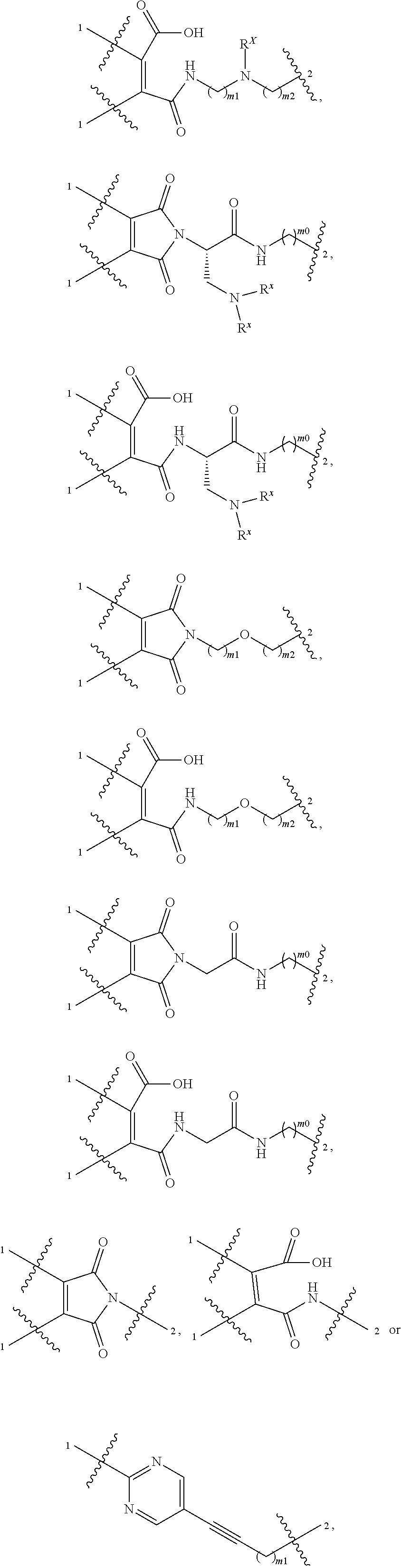

wherein position “1” is connected to Ab, and position “2” is connected to G^{L},
each m0 is independently 0, 1, 2, 3, 4 or 5;
m1 and m2 are each independently 1, 2, 3, 4 or 5;
each R^{x} is independently H or C₁₋₆ alkyl;
G^{L} is

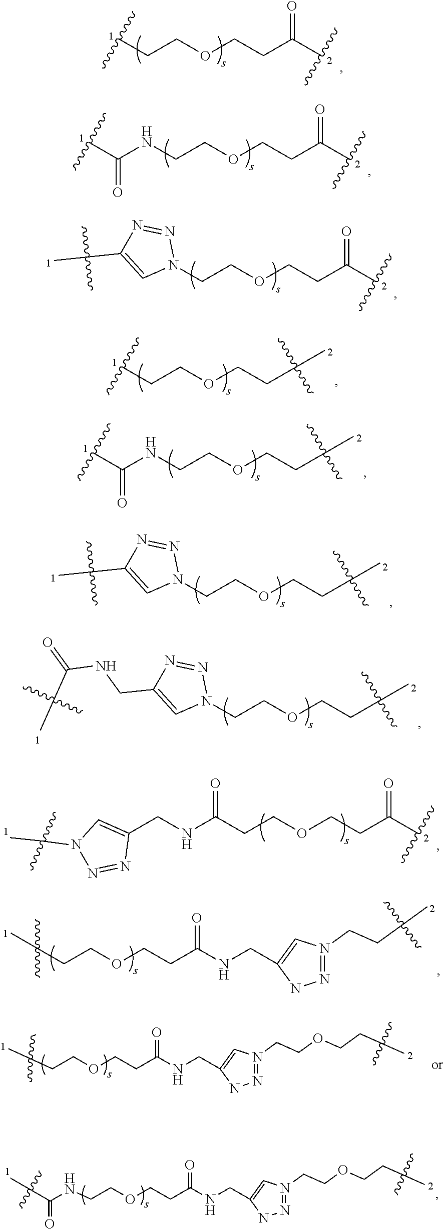

wherein position “1” is connected to G^{T}; position “2” is connected to G^{E};
each s is independently 1, 2, 3, 4, 5 or 6;
G^{E} is

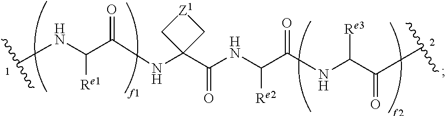

Z¹ is —CH₂— or O;
R^{e1}, R^{e2} and R^{e3} are independently H or —(CH₂)_{f2}—R^{ee}, R^{ee} is —OH, —COOH, —NH₂, —NR^{ee-1}R^{ee-2},

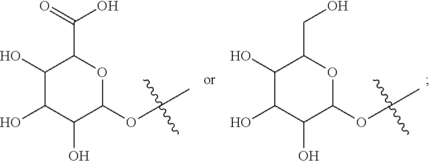 [SMILES: *OC1OC(C(=O)O)C(O)C(O)C1O ; *OC1OC(CO)C(O)C(O)C1O]

R^{ee-1} and R^{ee-2} are independently methyl, ethyl, n-propyl, isopropyl, n-butyl, isobutyl, sec-butyl or tert-butyl, f1 and f2 are independently 1, 2, 3 or 4;
D is

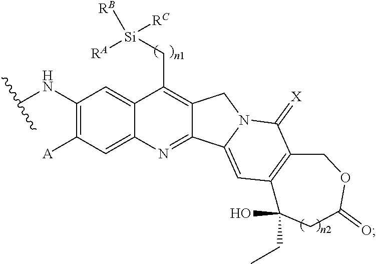

A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8;
preferably, the antibody is a Trastuzumab antibody, an FG1 antibody or an FG1-Tsc thiomab antibody;
G^{T} is

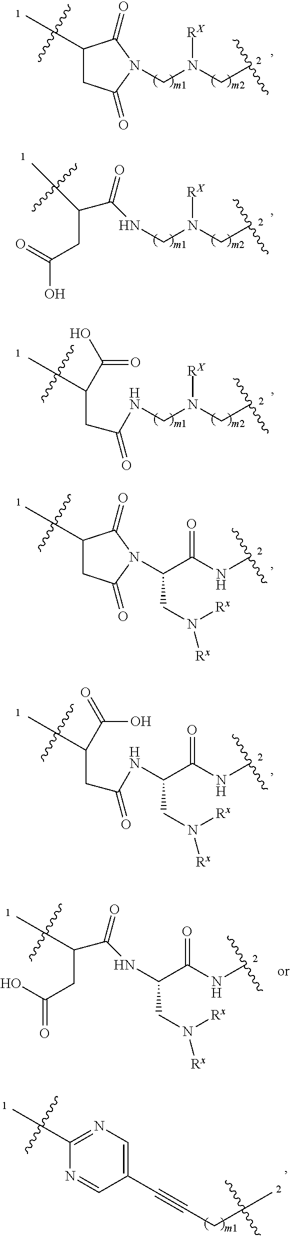

wherein position “1” is connected to Ab, and position “2” is connected to G^{L};
m1 and m2 are each independently 1, 2, 3, 4 or 5;
each R^{x} is independently H or C₁₋₆ alkyl;
G^{L} is

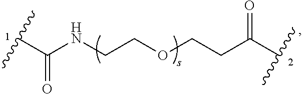

wherein position “1” is connected to G^{T}; position “2” is connected to G^{E};
s is 1, 2, 3, 4, 5 or 6;
G^{E} is

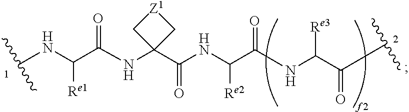

Z¹ is —CH₂— or O;
R^{e1}, R^{e2} and R^{e3} are independently H or —(CH₂)_{f2}—R^{ee}, R^{ee} is —OH, —COOH, —NH₂, —NR^{ee-1}R^{ee-2},

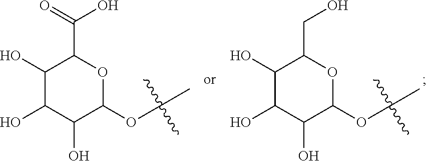 [SMILES: *OC1OC(C(=O)O)C(O)C(O)C1O ; *OC1OC(CO)C(O)C(O)C1O]

R^{ee-1} and R^{ee-2} are independently methyl, ethyl, n-propyl, isopropyl, n-butyl, isobutyl, sec-butyl or tert-butyl, f2 is 1 or 2;
D is

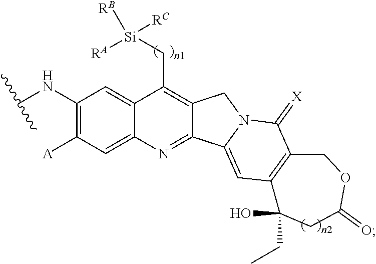

A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8.

In a certain embodiment, the drug conjugate of formula II is a drug conjugate of formula IIb,
the antibody is an antibody selected from one or more of anti-Her2 antibodies, anti-folate receptor α antibodies, anti-MUC1 antibodies and anti-human IgG1 antibodies;
G^{T} is

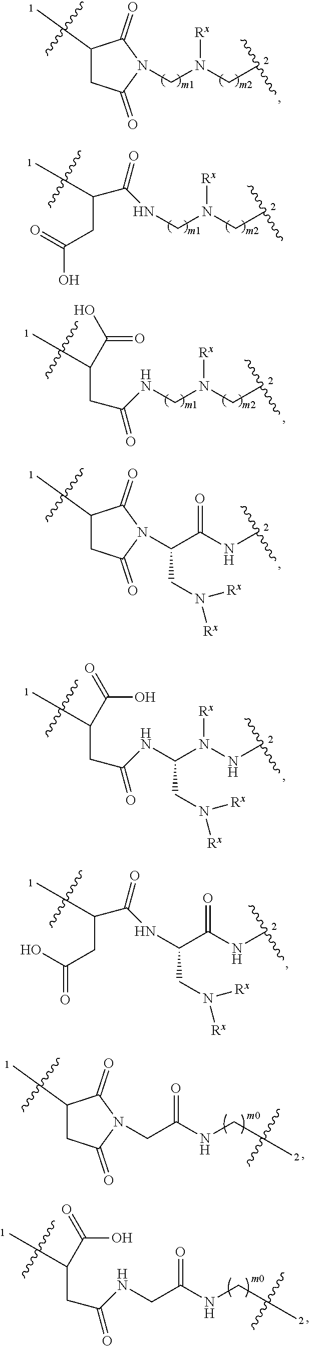

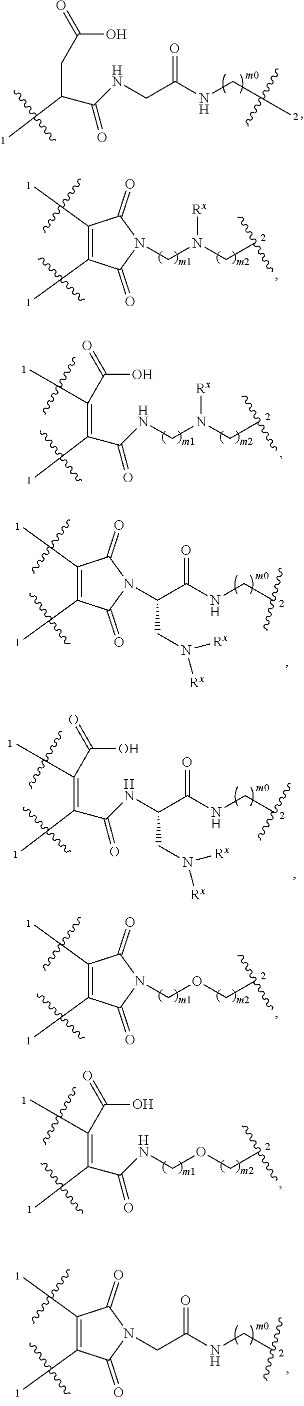

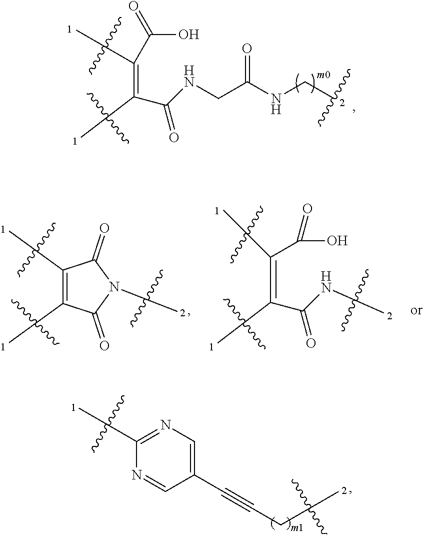

wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl;
G^{L} is -G^{L1}- or -G^{L1}-G^{L2}-G^{L1} is connected to G^{T};
G^{L1} is

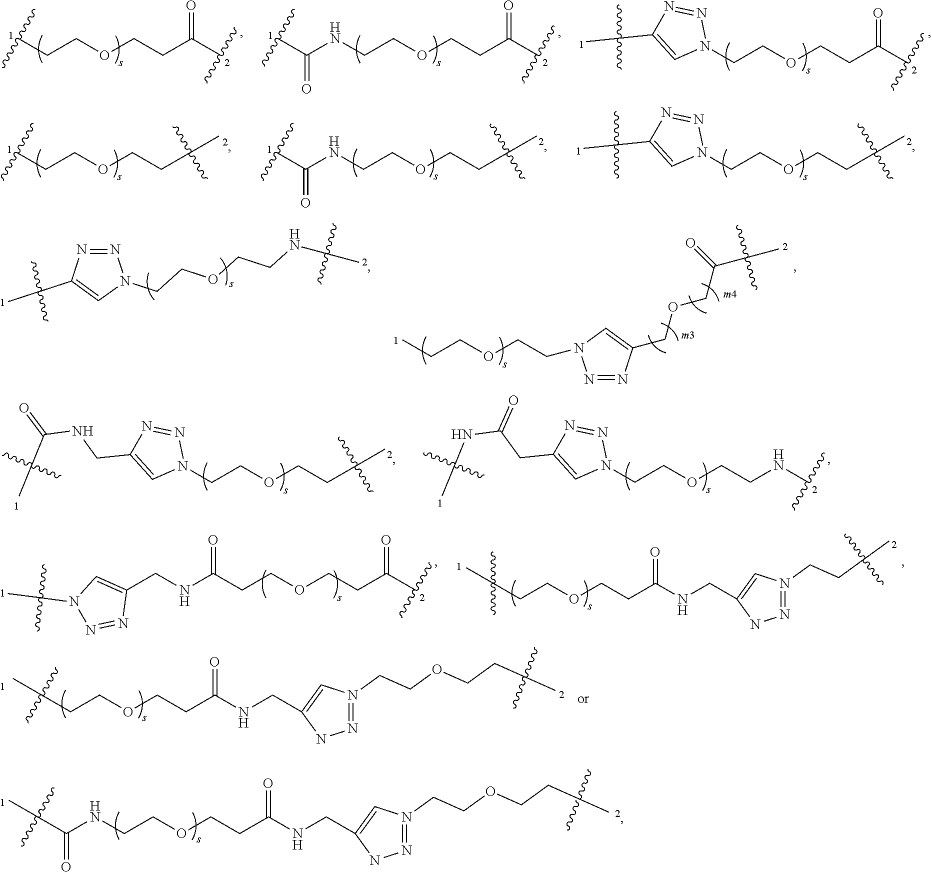

wherein position “1” is connected to G^{T}, and position “2” is connected to G^{L2} or G^{s};
each s is independently 1, 2, 3, 4, 5 or 6;
G^{L2} is

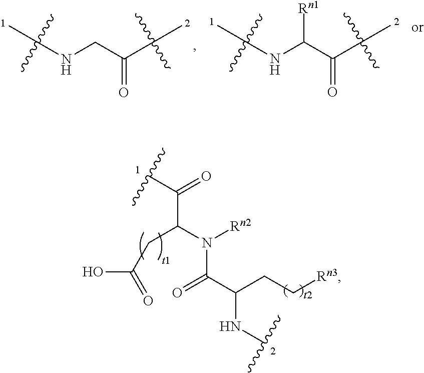

wherein position “1” is connected to G^{L1}; position “2” is connected to G^{E} or G^{S}; t1 and t2 are independently 1, 2, 3 or 4; Rⁿ¹ is C₁₋₄ alkyl, Rⁿ² is H or methyl, and Rⁿ³ is amino,

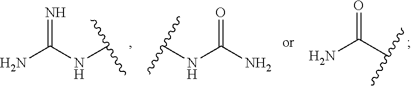

G^{S} is

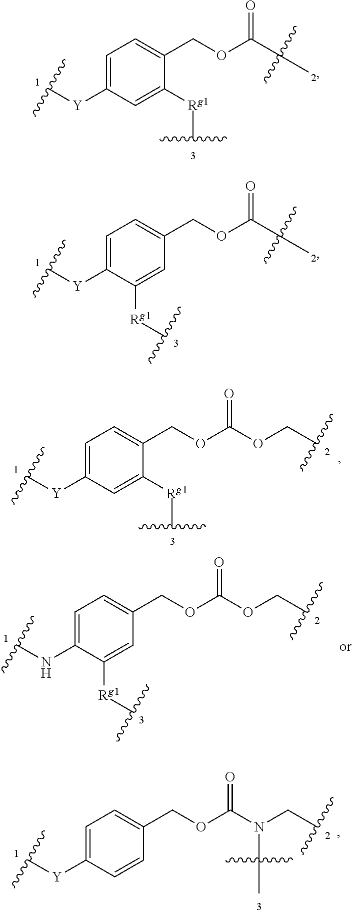

wherein each R^{g1} is independently

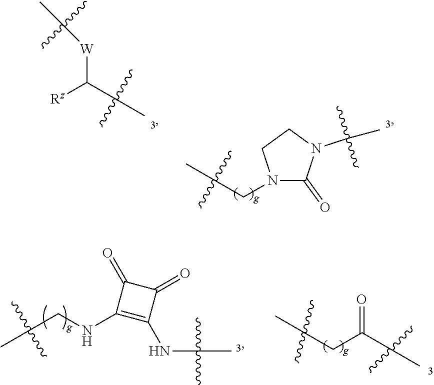

or —NH—³; wherein position “1” is connected to G^{I}, position “2” is connected to A^{m}, and position “3” is connected to G^{L};
each Y is independently NH or a single bond;
each W is independently C₁₋₆ alkylene or C₂₋₆ alkynylene;
each R^{z} is independently

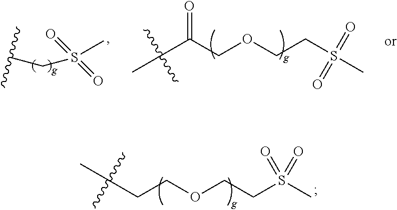

each g is independently 1, 2, 3, 4, 5 or 6;
G^{I} is

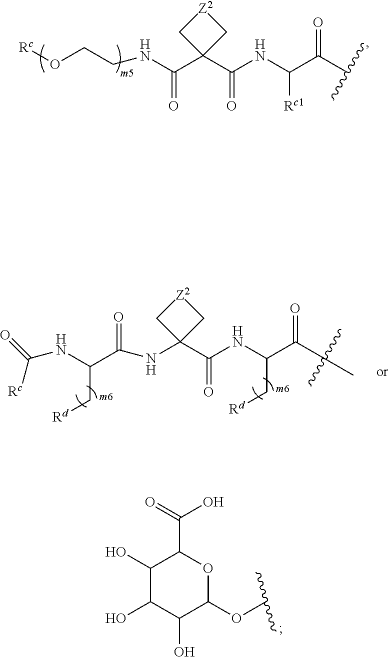

Z² is —CH₂— or O;
R^{c} is C₁₋₆ alkyl;
R^{c1} is

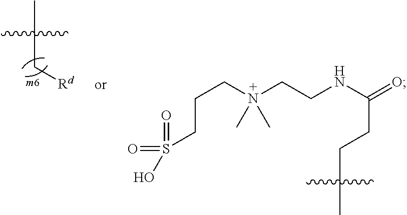

R^{d} is hydroxyl,

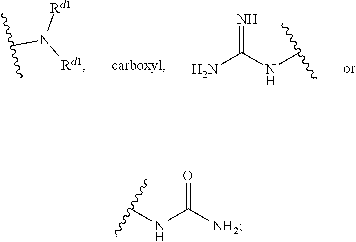

each R^{d1} is independently H or C₁₋₆ alkyl;
m5 and m6 are independently 1, 2, 3, 4, 5 or 6;
A^{m} is a single bond or

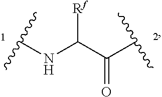

R^{f} is H, C₁₋₆ alkyl or —(CH₂)ₙ—R^{f1}, R^{f1} is —NH₂ or —NR^{ee-1}R^{ee-2} n is 1, 2, 3, 4, 5 or 6, R^{ee-1} and R^{ee-2} are independently methyl, ethyl, n-propyl, isopropyl, n-butyl, isobutyl, sec-butyl or tert-butyl, wherein position “1” is connected to G^{S}, and position “2” is connected to D,
D is

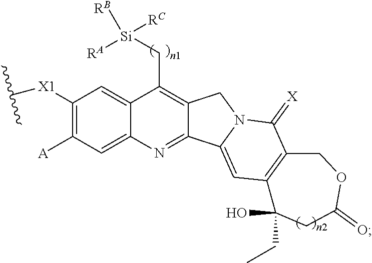

X1 is NH or O;
A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8.

In a certain embodiment, in the drug conjugate of formula IIb, R^{d} is hydroxyl or

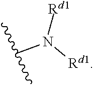

In a certain embodiment, in the drug conjugate of formula IIb, R^{d} is carboxyl,

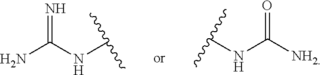 [SMILES: *NC(=N)N ; *NC(N)=O]

In a certain embodiment, the drug conjugate of formula IIb is a drug conjugate of formula IIb-1 or IIb-2,

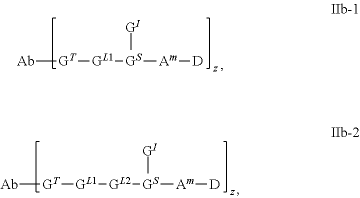

Ab, G^{T}, G^{L1}, G^{L2}, G^{S}, G^{I}, A^{m}, D, and z are defined as described in any one embodiment of the present disclosure;
preferably, in the drug conjugate of formula IIb-1, G^{S} is

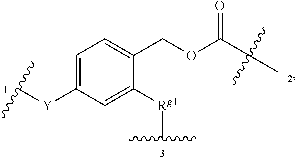

and R^{g1} is

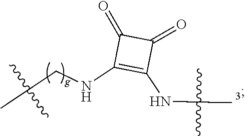

or, in the drug conjugate of formula IIb-2, G^{S} is

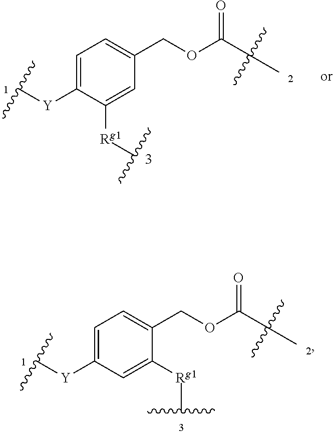

and R^{g1} is

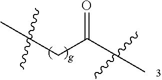

or —NH—³; wherein position “1” is connected to G^{I}, position “2” is connected to A^{m}, and position “3” is connected to G^{L}.

In a certain embodiment, in the drug conjugate of formula IIb-1, G^{T} is

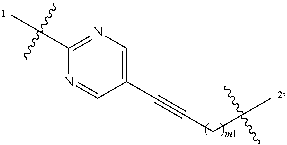

### and G^{L1} is

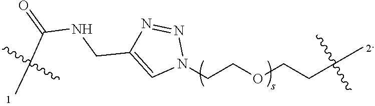

In a certain embodiment, in the drug conjugate of formula IIb-2, G^{T} is

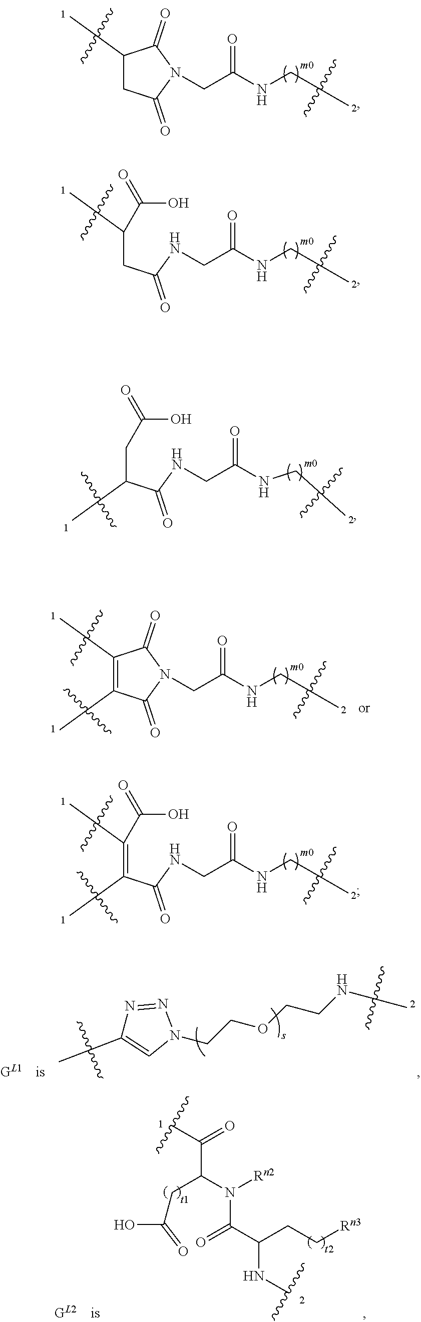

### and R^{g1} is

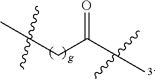 [SMILES: *CC(*)=O]

In a certain embodiment, in the drug conjugate of formula IIb-2, G^{T} is

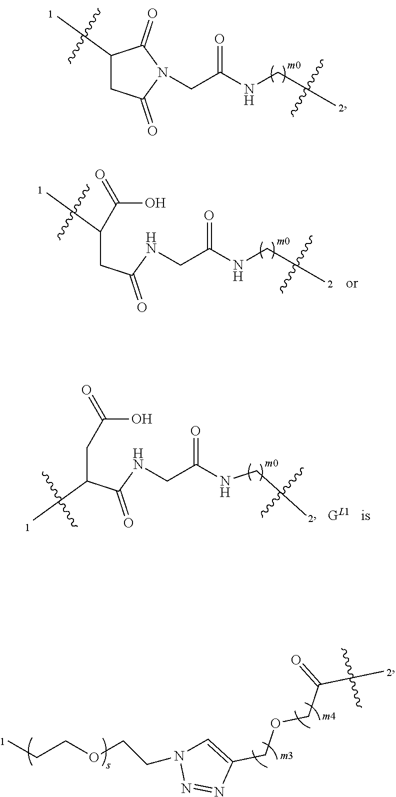

### and G^{L2} is

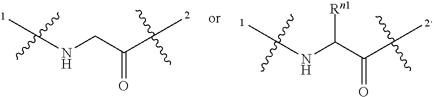

In a certain embodiment, the drug conjugate of formula II is a drug conjugate of formula IIc,
Ab is an antibody selected from one or more of anti-Her2 antibodies, anti-folate receptor α antibodies anti-MUC1 antibodies and anti-human IgG1 antibodies;
G^{T} is

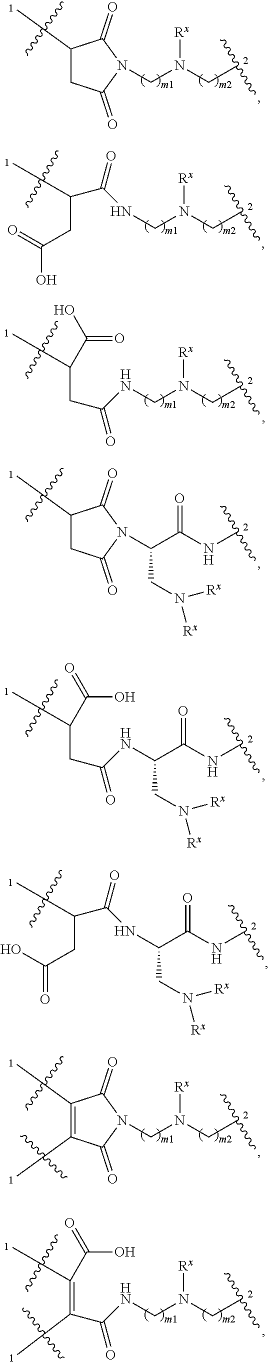

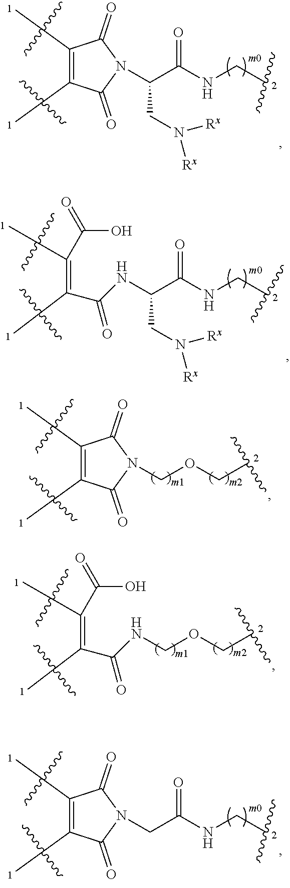

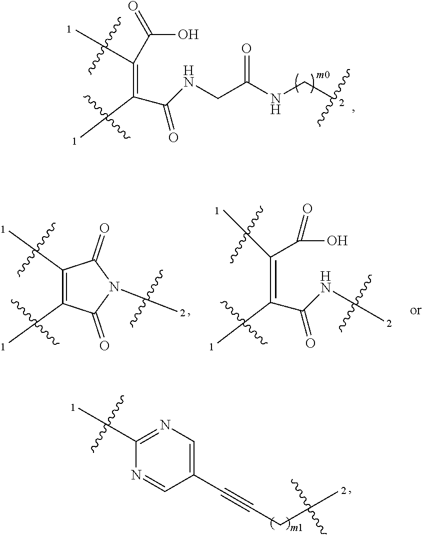

wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl;
G^{L} is

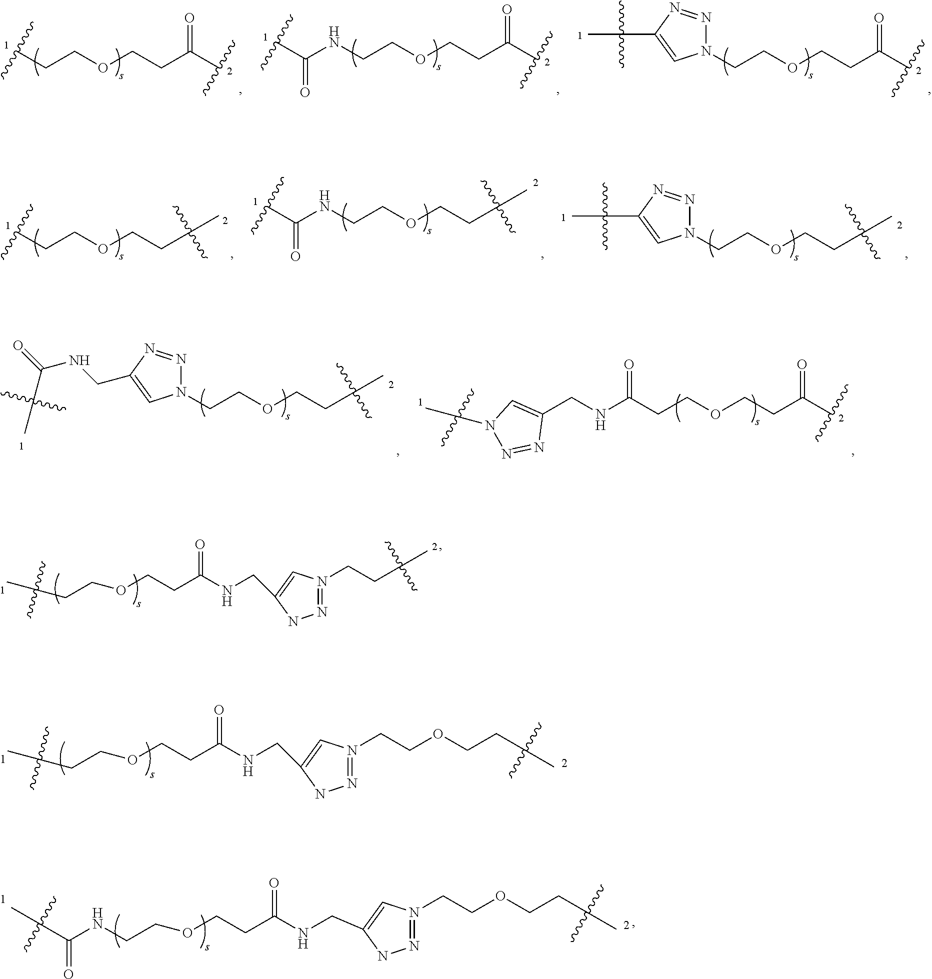

wherein position “1” is connected to G^{T}; position “2” is connected to G^{E};
each s is independently 1, 2, 3, 4, 5 or 6;
G^{E} is

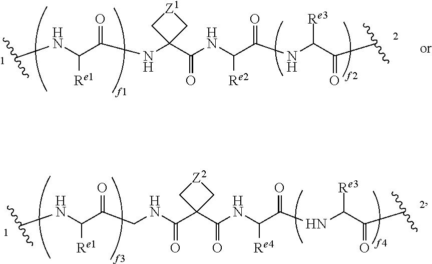

Z¹ and Z² are independently —CH₂— or O;
R^{e1}, R^{e2}, R^{e3} and R^{e4} are independently H or —(CH₂)_{f5}—R^{ee},
R^{ee} is —OH, —COOH, —NH₂, —NR^{ee-1}R^{ee-2},

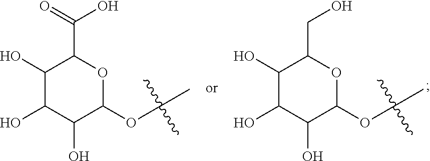 [SMILES: *OC1OC(C(=O)O)C(O)C(O)C1O ; *OC1OC(CO)C(O)C(O)C1O]

R^{ee-1} and R^{ee-2} are independently methyl ethyl, n-propy, isopropyl, n-butyl, isobutyl, sec-butyl or tert-butyl;
f1 and f2 are independently 1, 2, 3 or 4; f3 and f4 are independently 0, 1, 2, 3 or 4;
f5 is 1, 2, 3, 4, 5 or 6;
G^{F} is

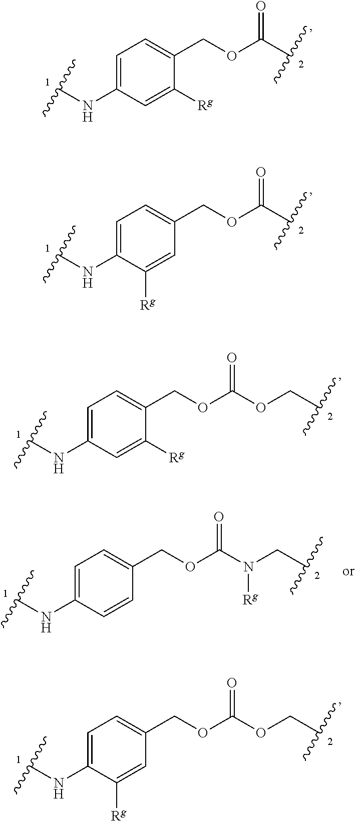

wherein position “1” is connected to G^{L}, and position “2” is connected to A^{m}.
each R^{g} is independently H,

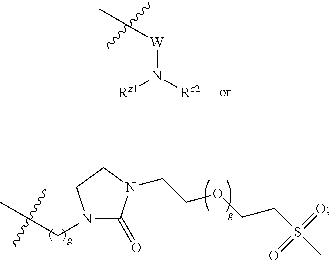

W is independently C₁₋₆ alkylene or C₂₋₆ alkynylene;
R^{z1} and R^{z2} are independently

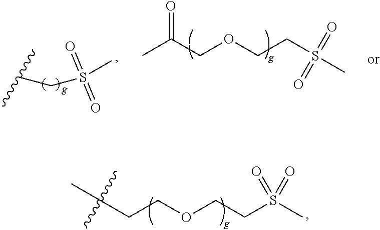

each g is independently 1, 2, 3, 4, 5 or 6;
A^{m} is a single bond or

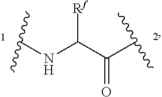

R^{f} is H, C₁₋₆ alkyl or —(CH₂)ₙ—R^{f1}, R^{f1} is —NH₂ or —NR^{ee-1}R^{ee-2} n is 1, 2, 3, 4, 5 or 6, R^{ee-1} and R^{ee-2} are independently methyl, ethyl, n-propyl, isopropyl, n-butyl, isobutyl, sec-butyl or tert-butyl, wherein position “1” is connected to G^{S}, and position “2” is connected to D,
D is

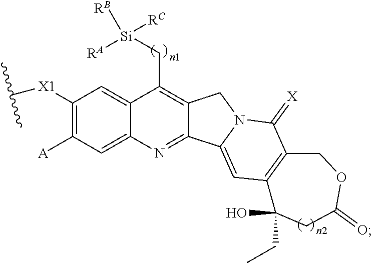

X1 is NH or O;
A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8.

In a certain embodiment, the drug conjugate of formula II is any one of the following drug conjugates or hydrolysates thereof:

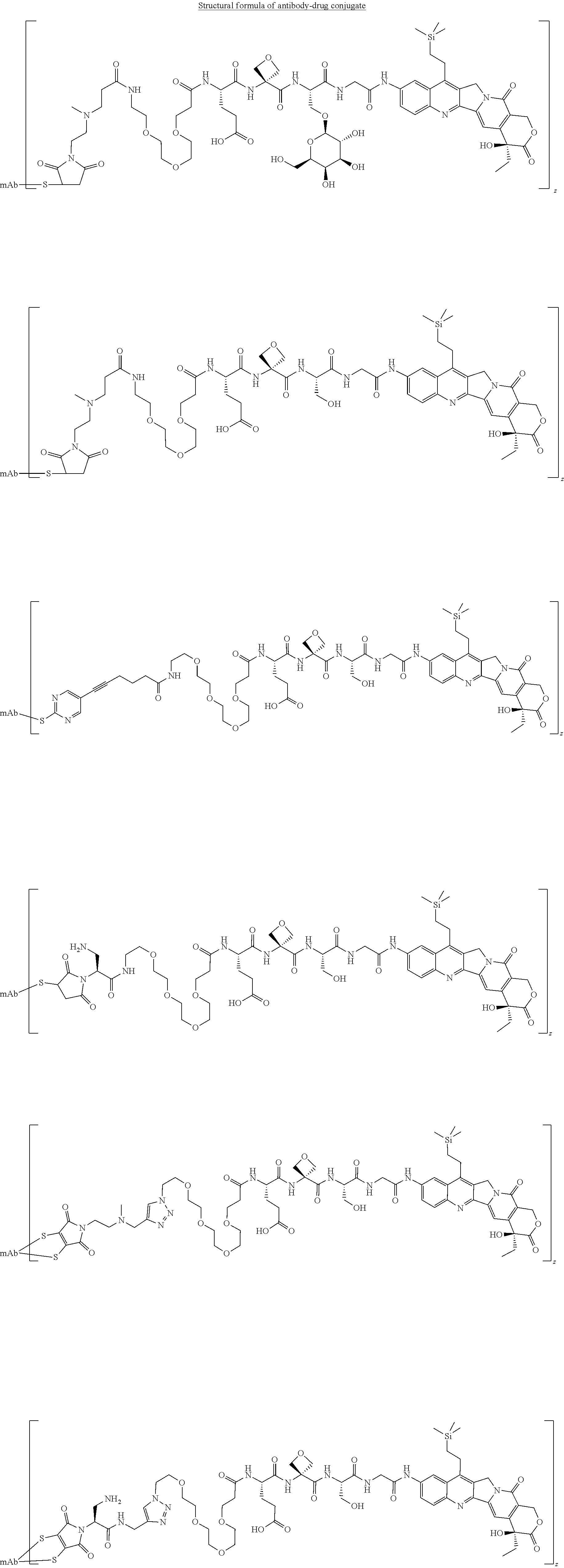

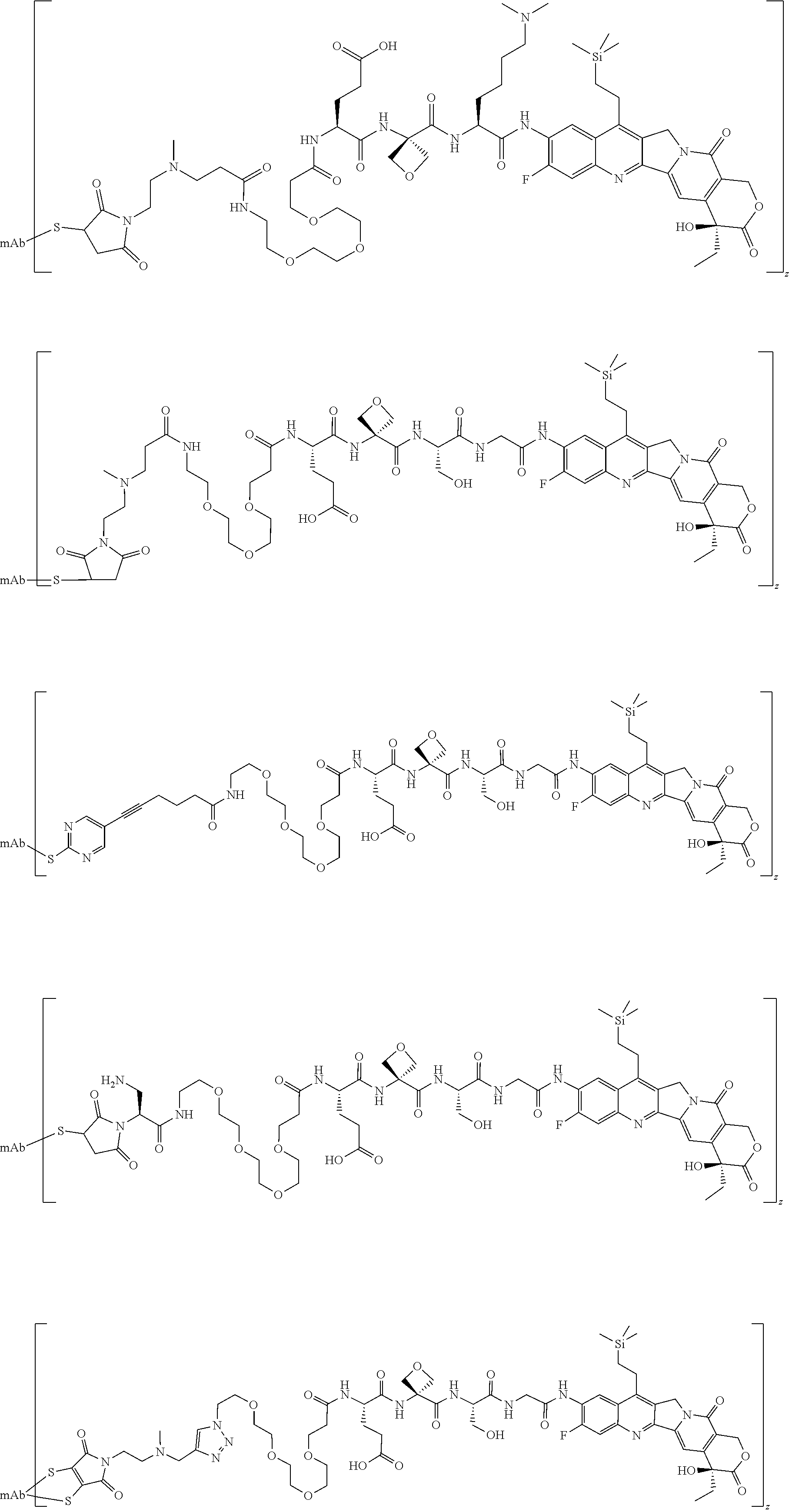

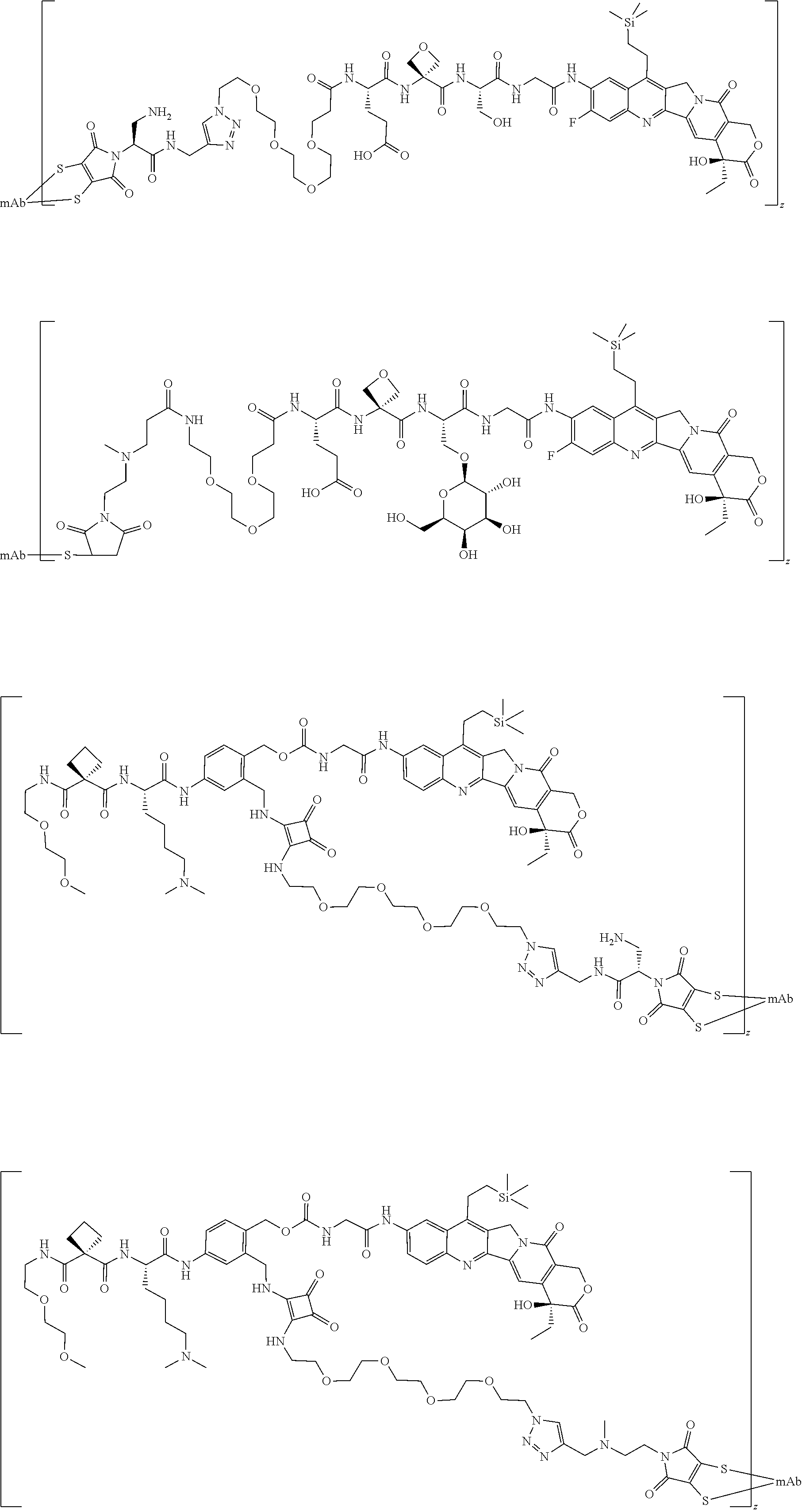

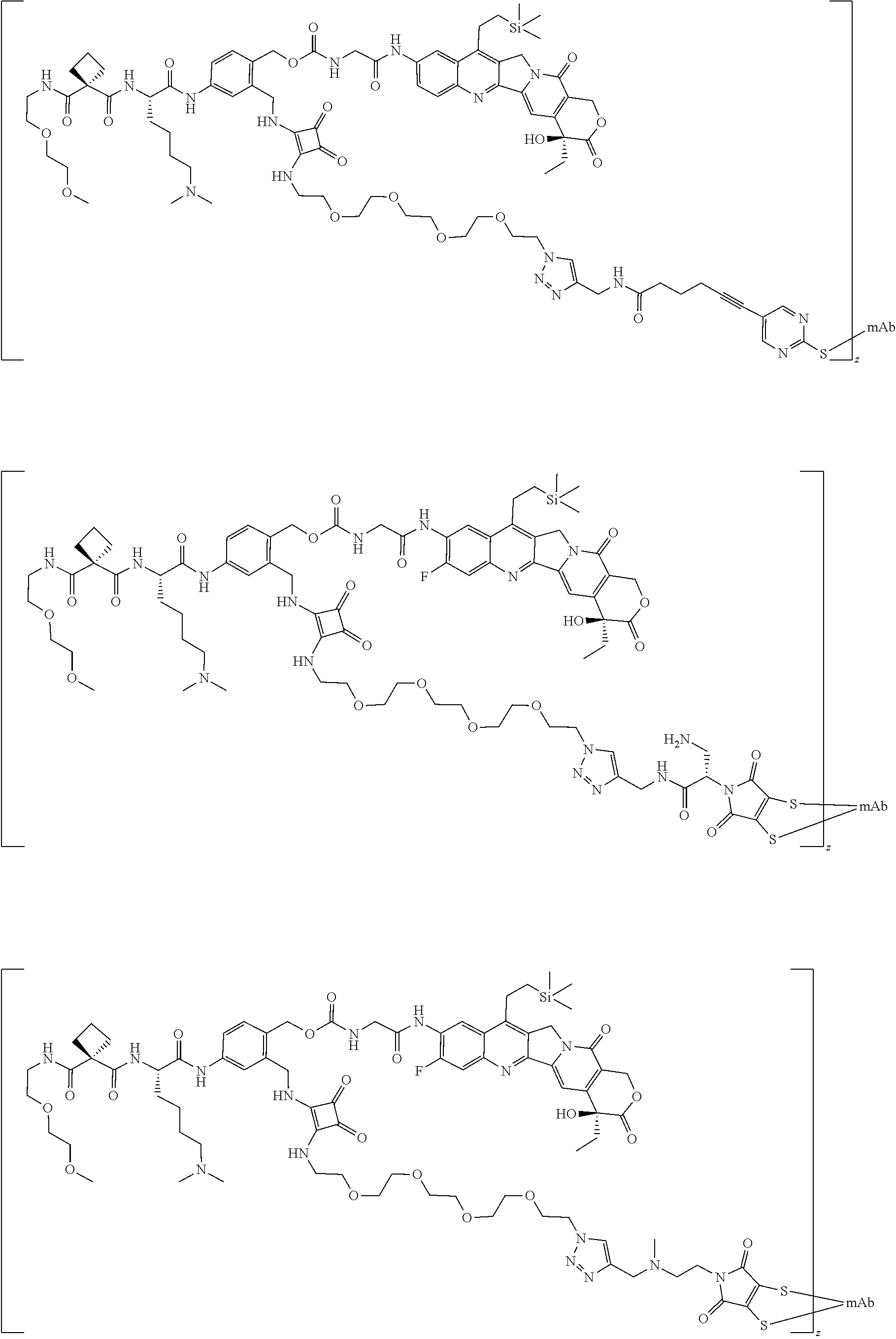

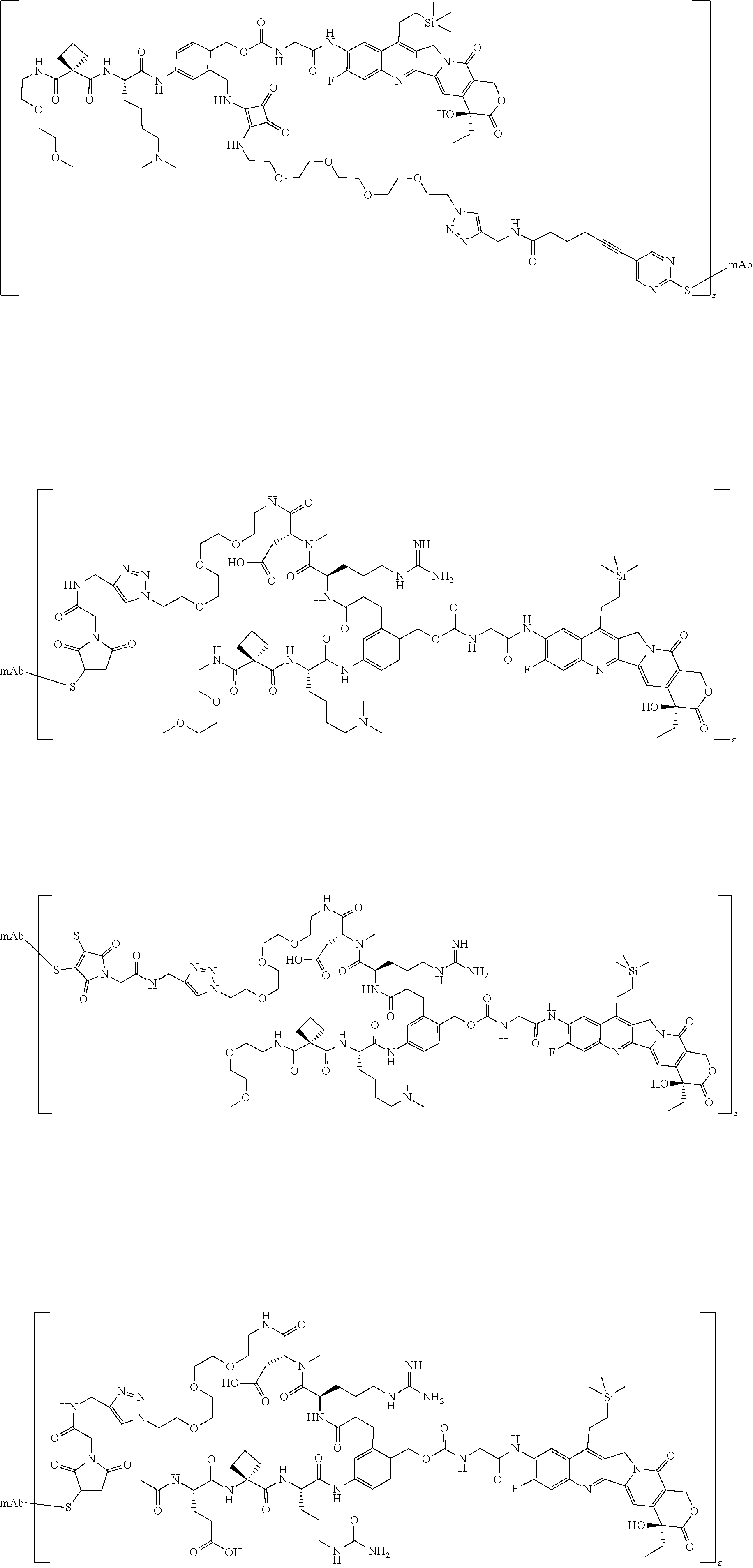

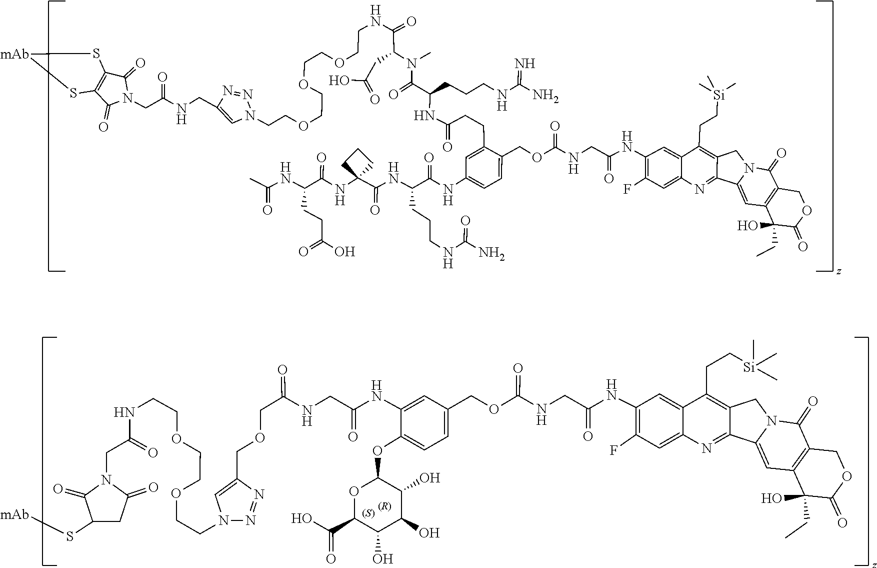

wherein the mAb is independently the Trastuzumab antibody, the FG1 antibody, the v30384 antibody, the Gatipotuzumab antibody, the DNP antibody, the FG1-Tsc antibody or the FG1-Tsc thiomab antibody, and z is independently an integer or decimal of 1-8;
preferably, the drug conjugate of formula II is any one of the following drug conjugates or hydrolysates thereof:

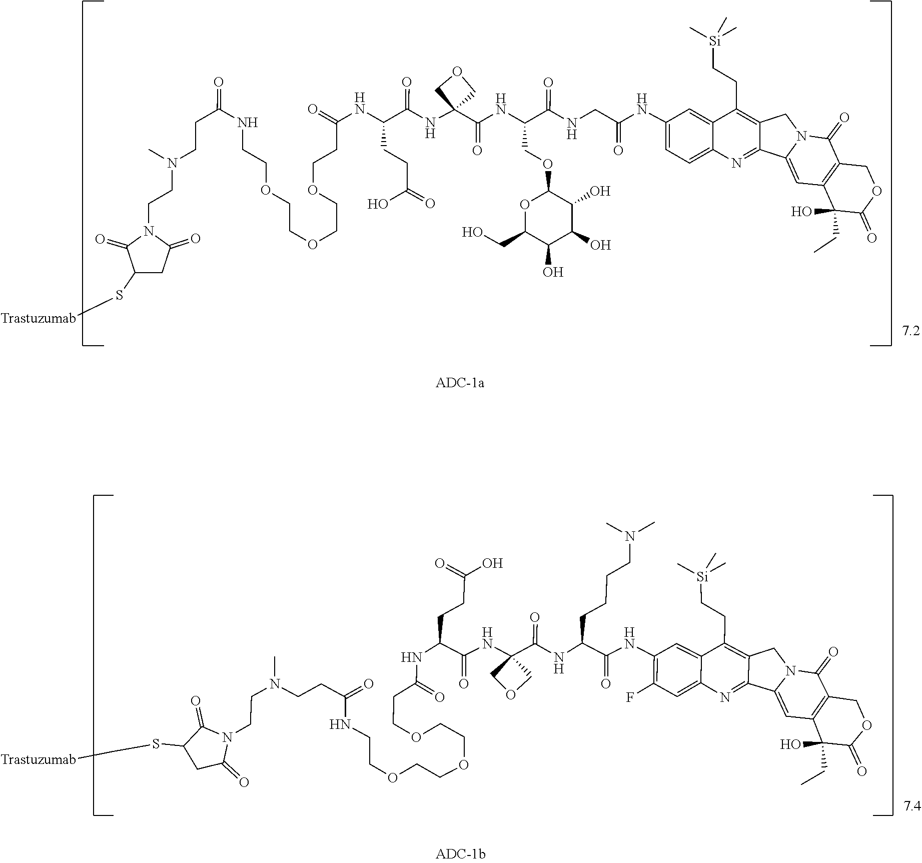

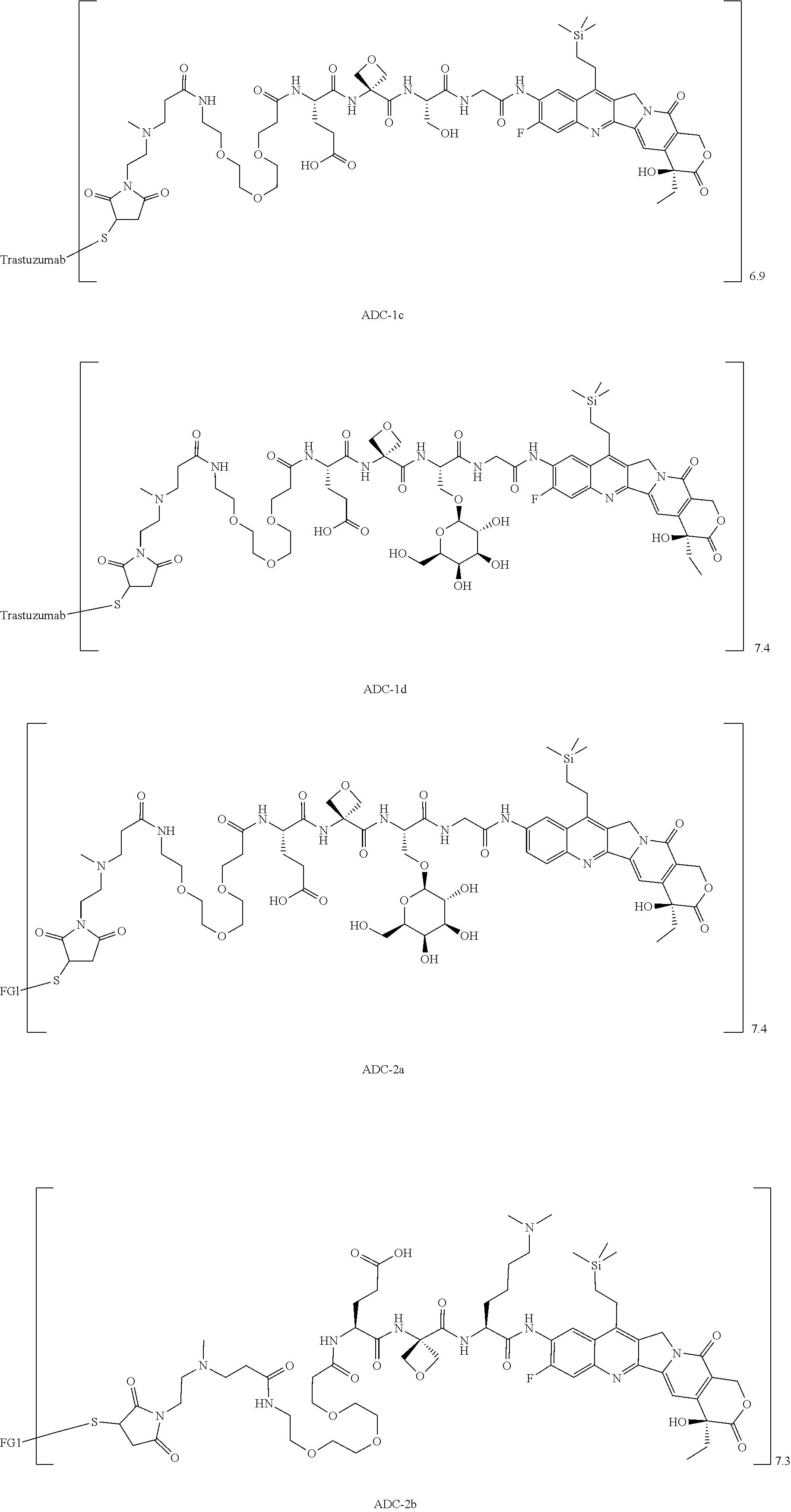

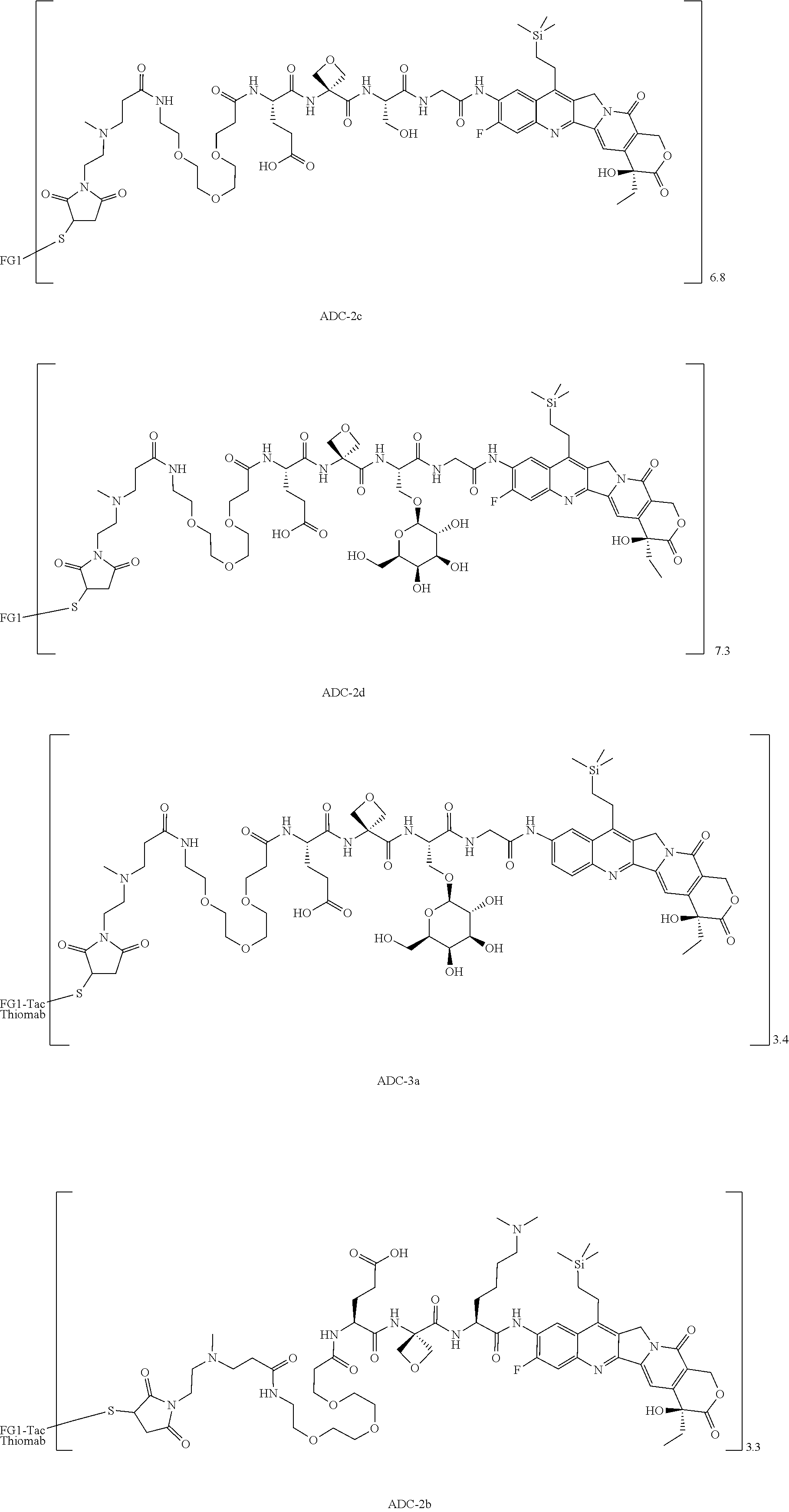

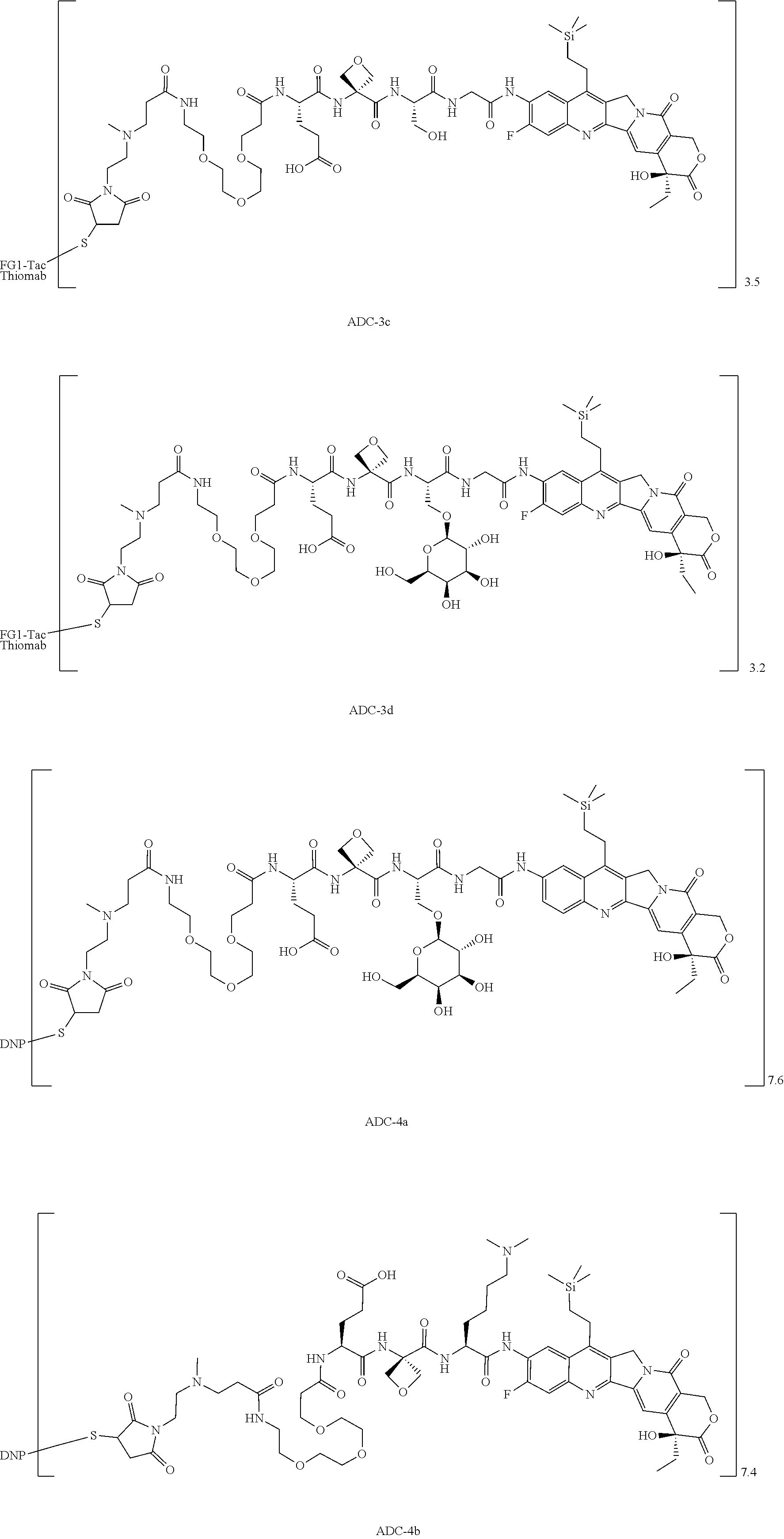

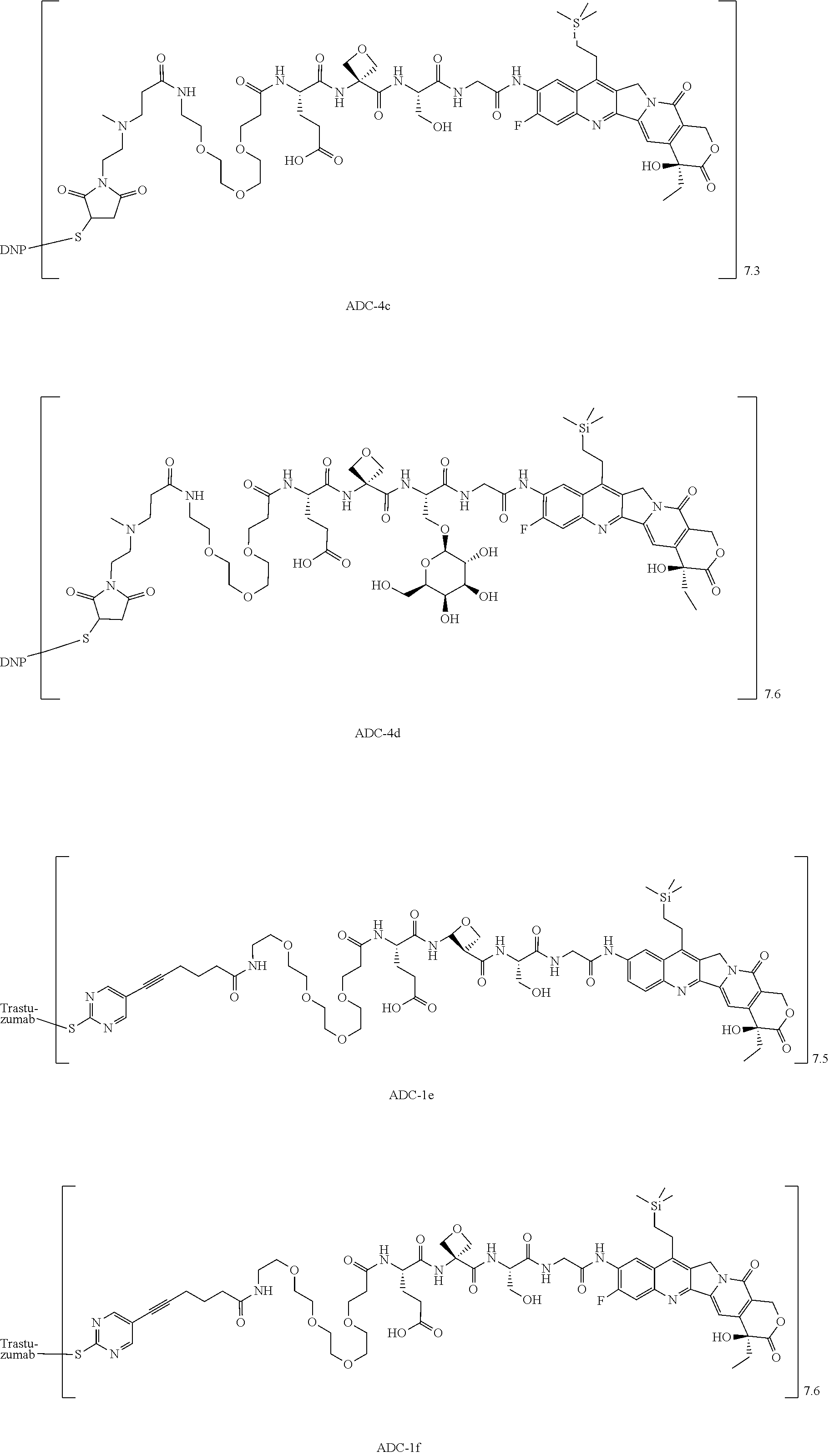

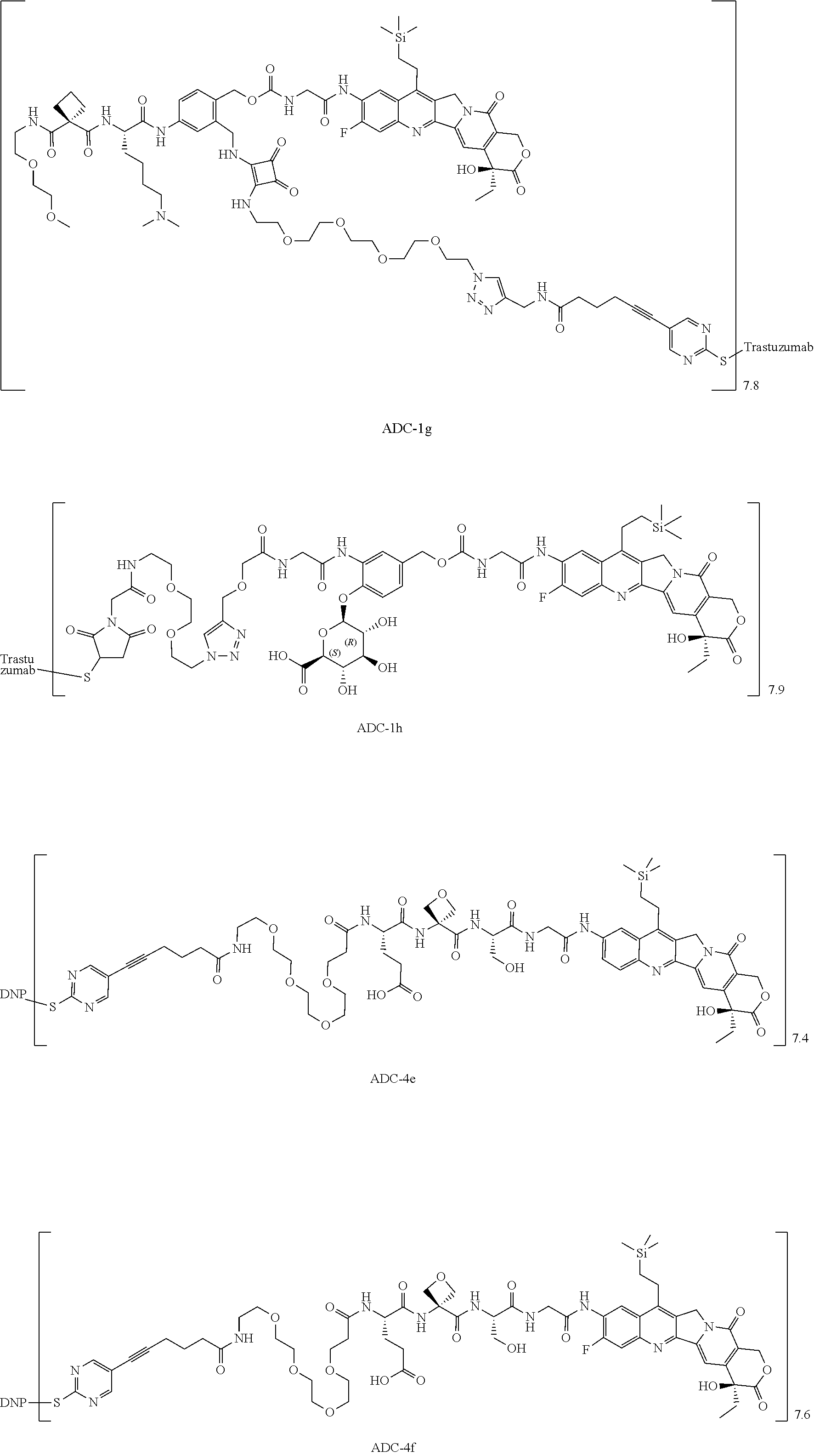

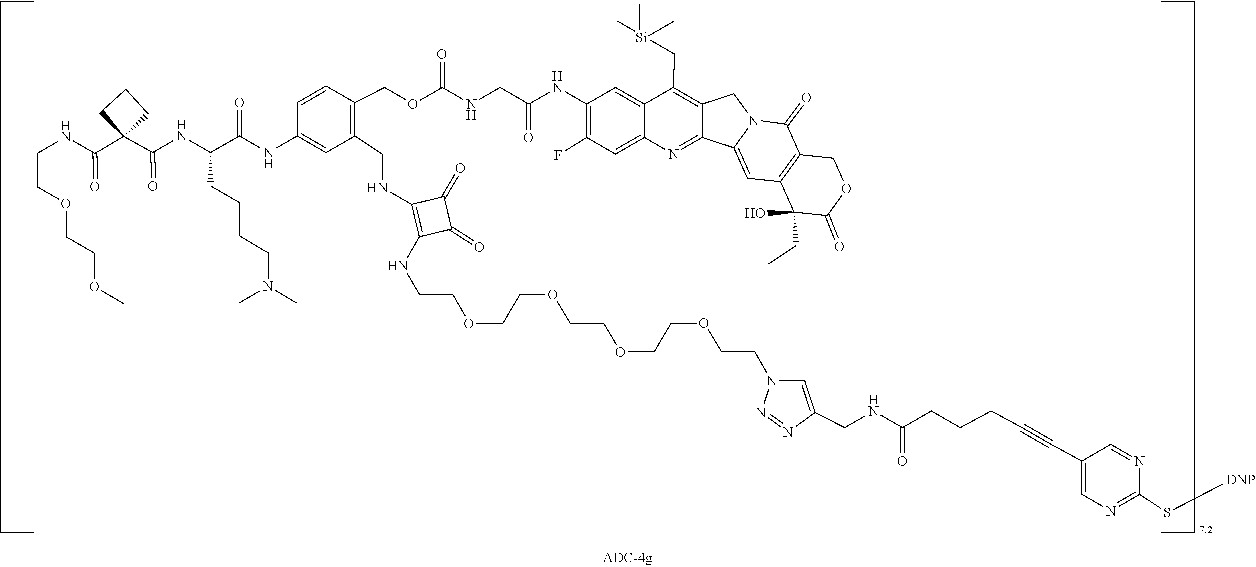

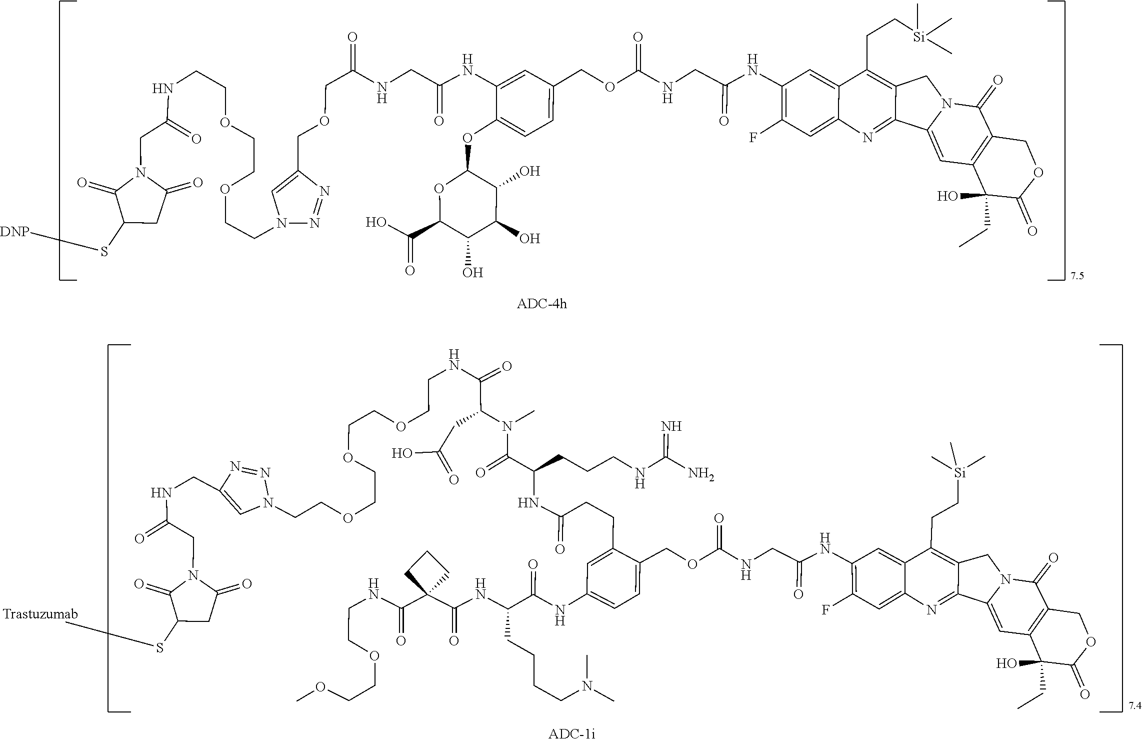

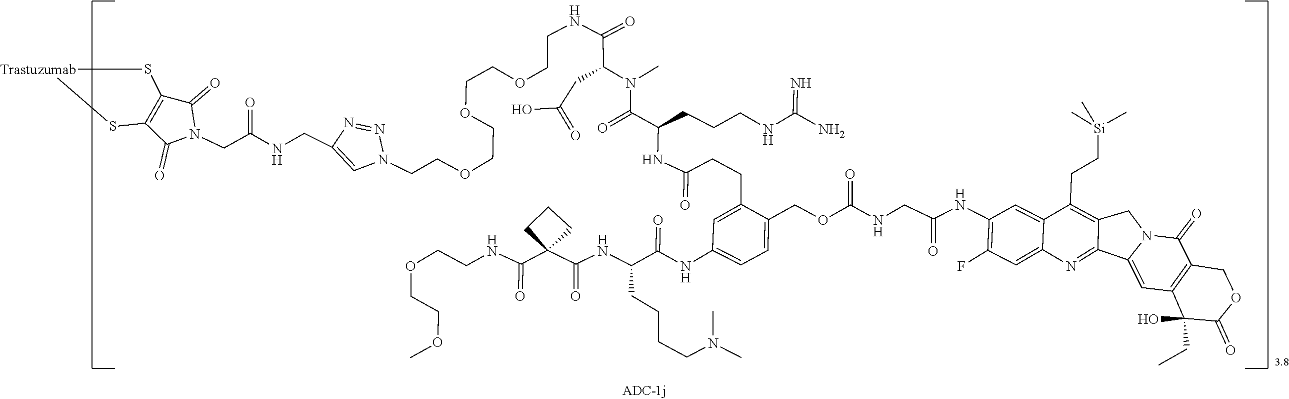

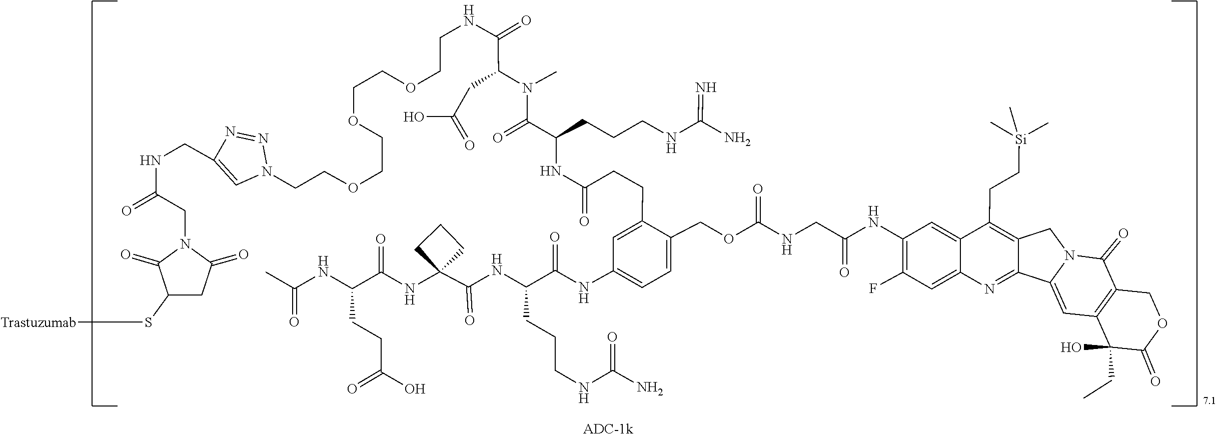

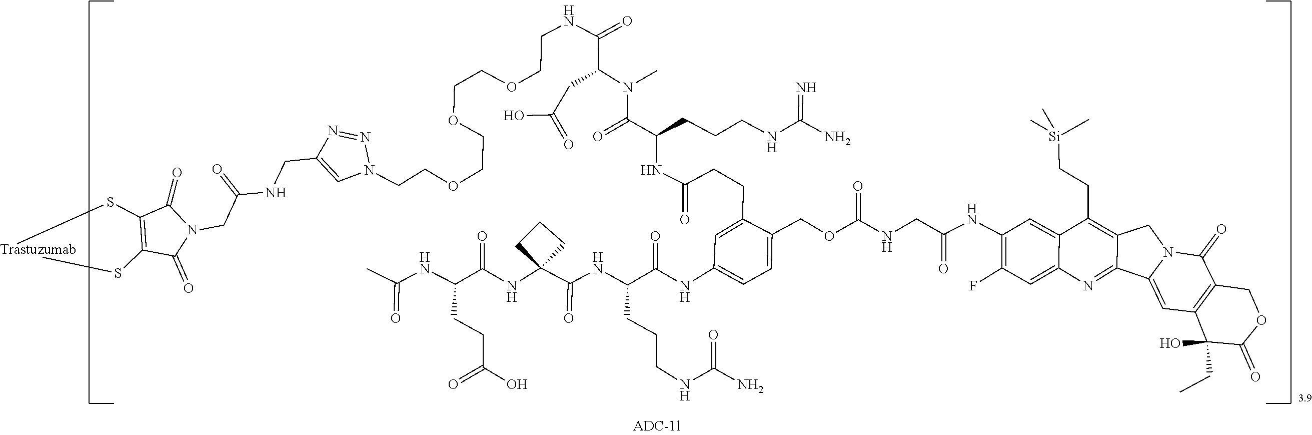

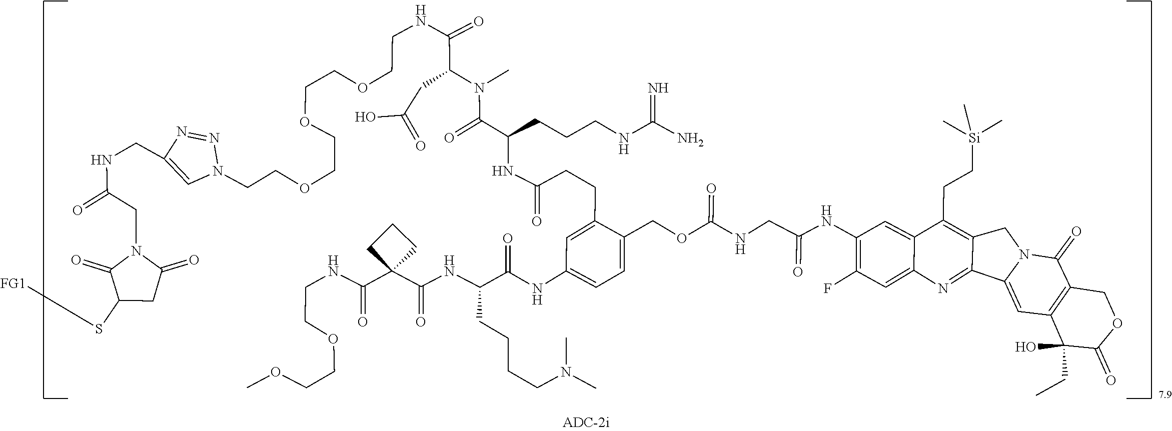

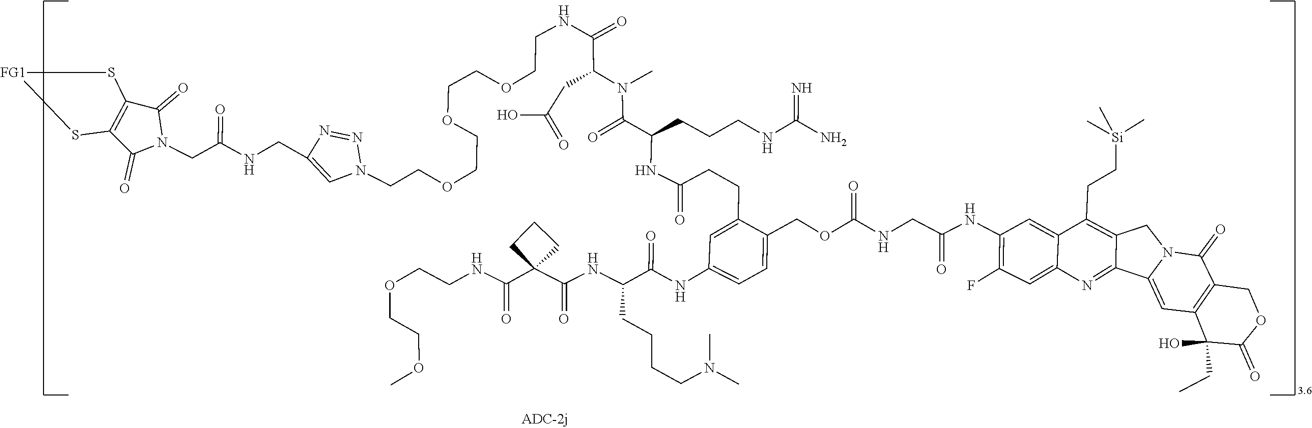

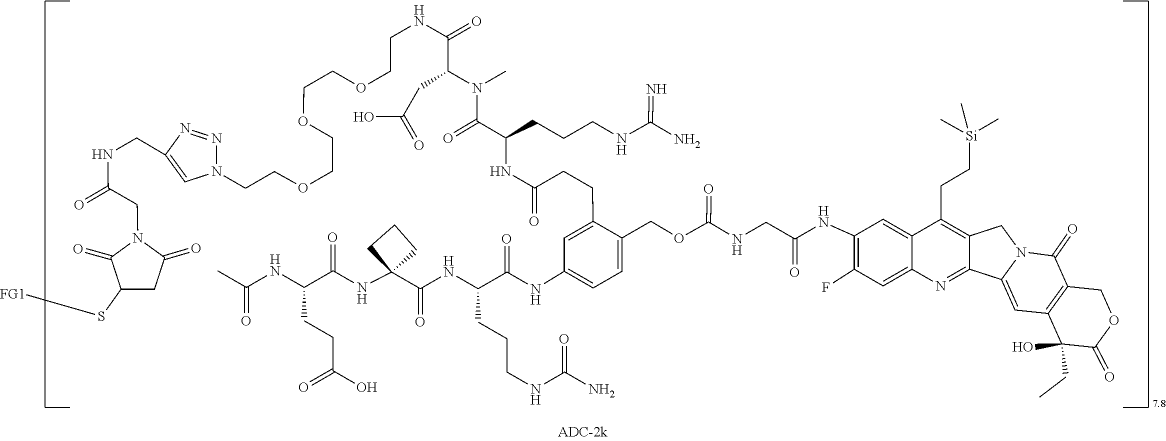

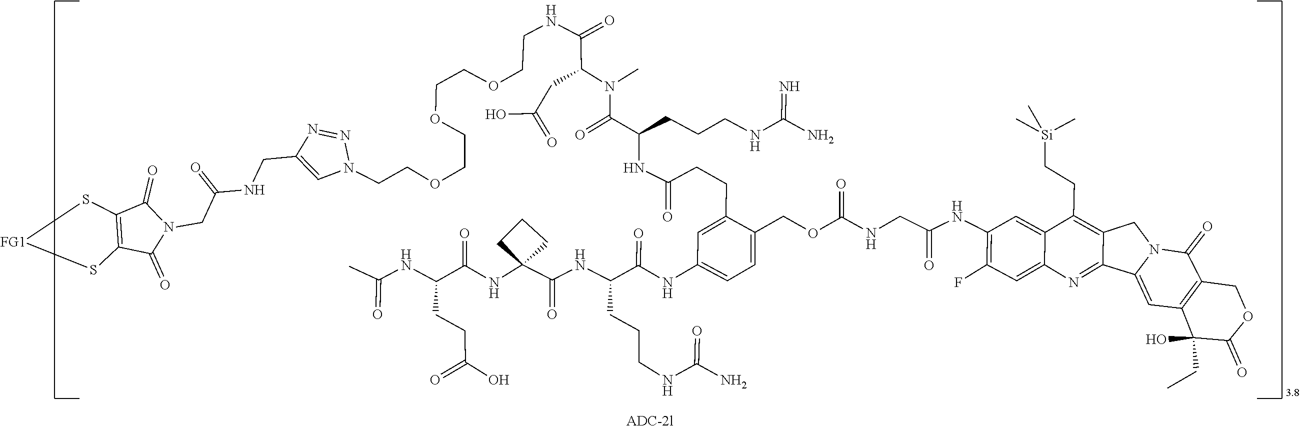

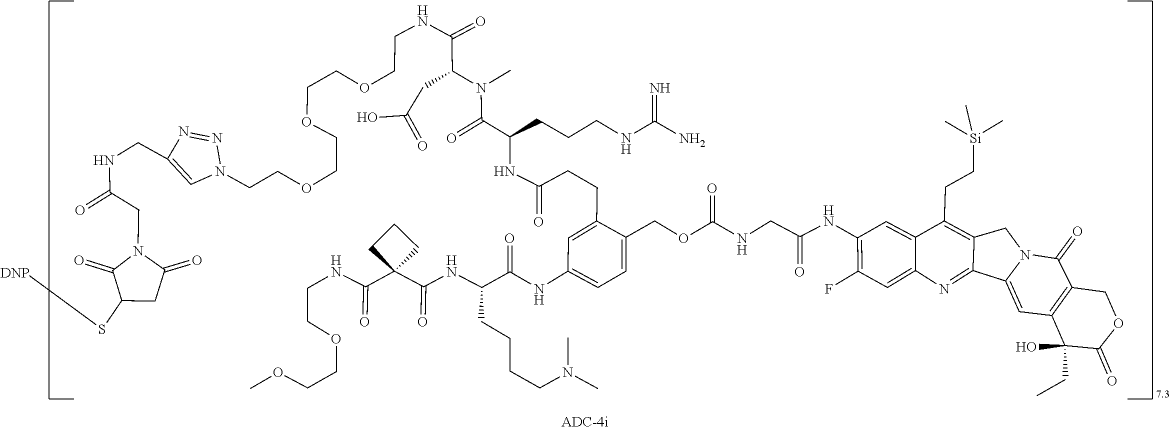

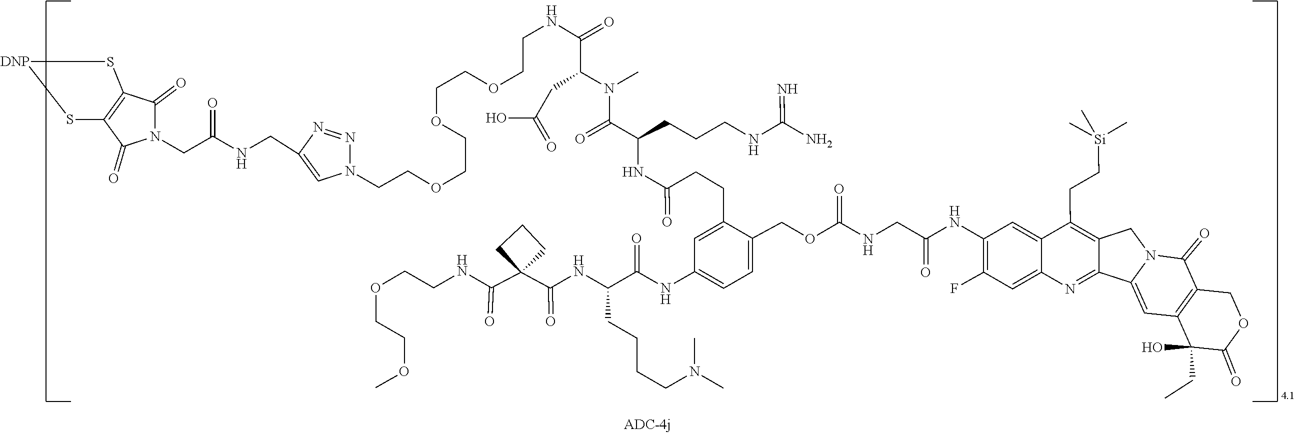

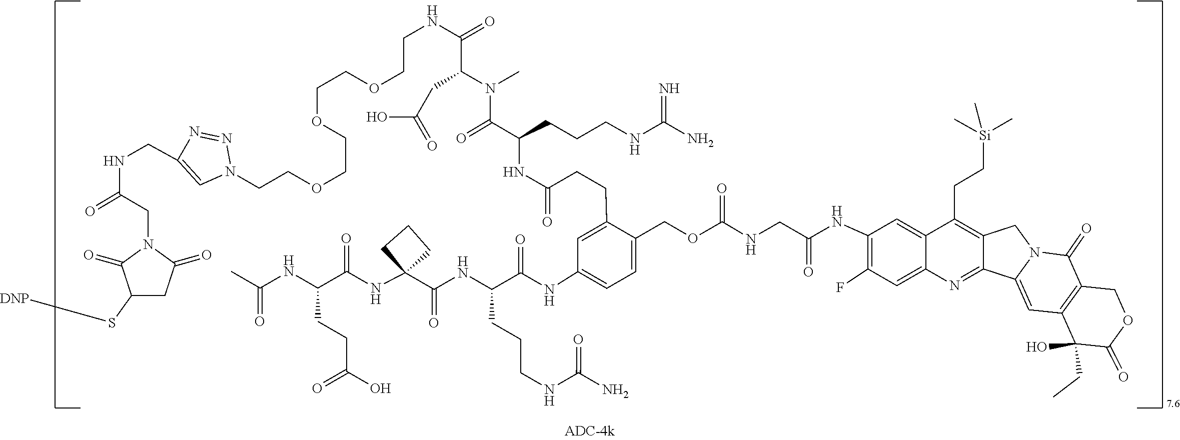

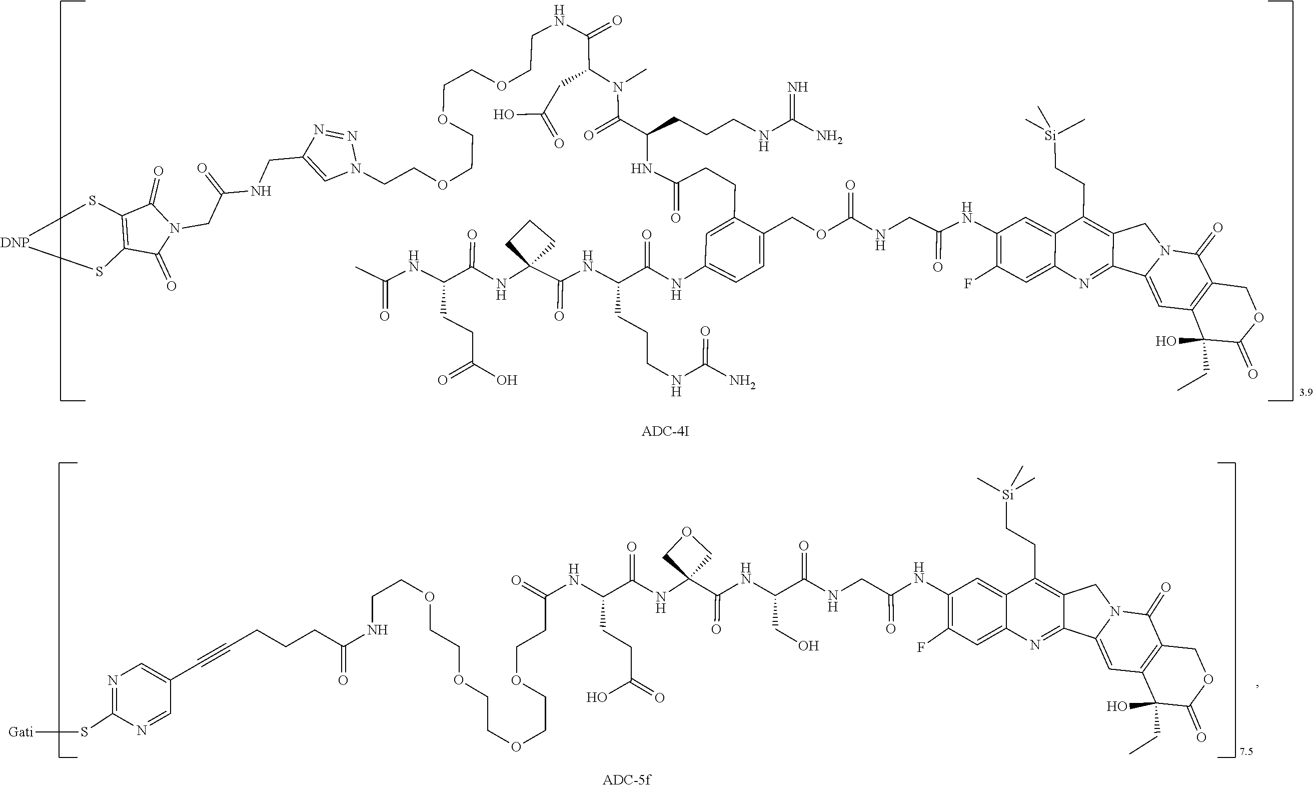

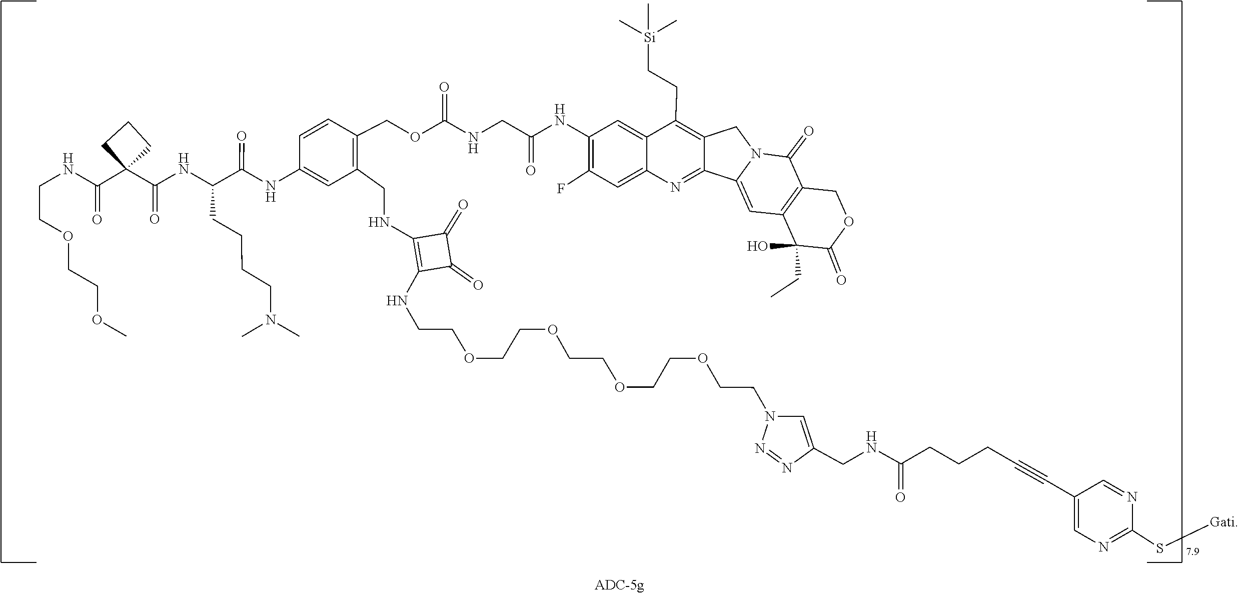

It should be noted that the hydrolysate of the drug conjugate of formula II as described above means that when the structure of the drug conjugate connected to the antibody is

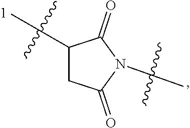 [SMILES: *C1CC(=O)N(*)C1=O]

the hydrolysate of the drug conjugate is a drug conjugate formed by replacing

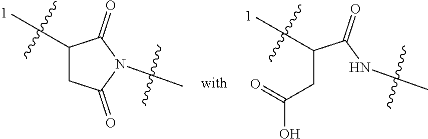 [SMILES: *C1CC(=O)N(*)C1=O ; *NC(=O)C(*)CC(=O)O]

(hydrolyzed form) and/or

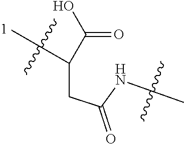 [SMILES: *NC(=O)CC(*)C(=O)O]

(hydrolyzed form), and when the structure of the drug conjugate connected to the antibody is

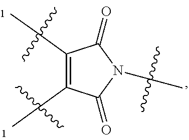 [SMILES: *C1=C(*)C(=O)N(*)C1=O]

the hydrolysate of the drug conjugate is a drug conjugate formed by replacing

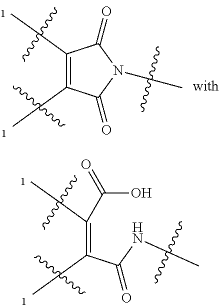

(hydrolyzed form), wherein position “1” is connected to the antibody. For example,
the hydrolysate of

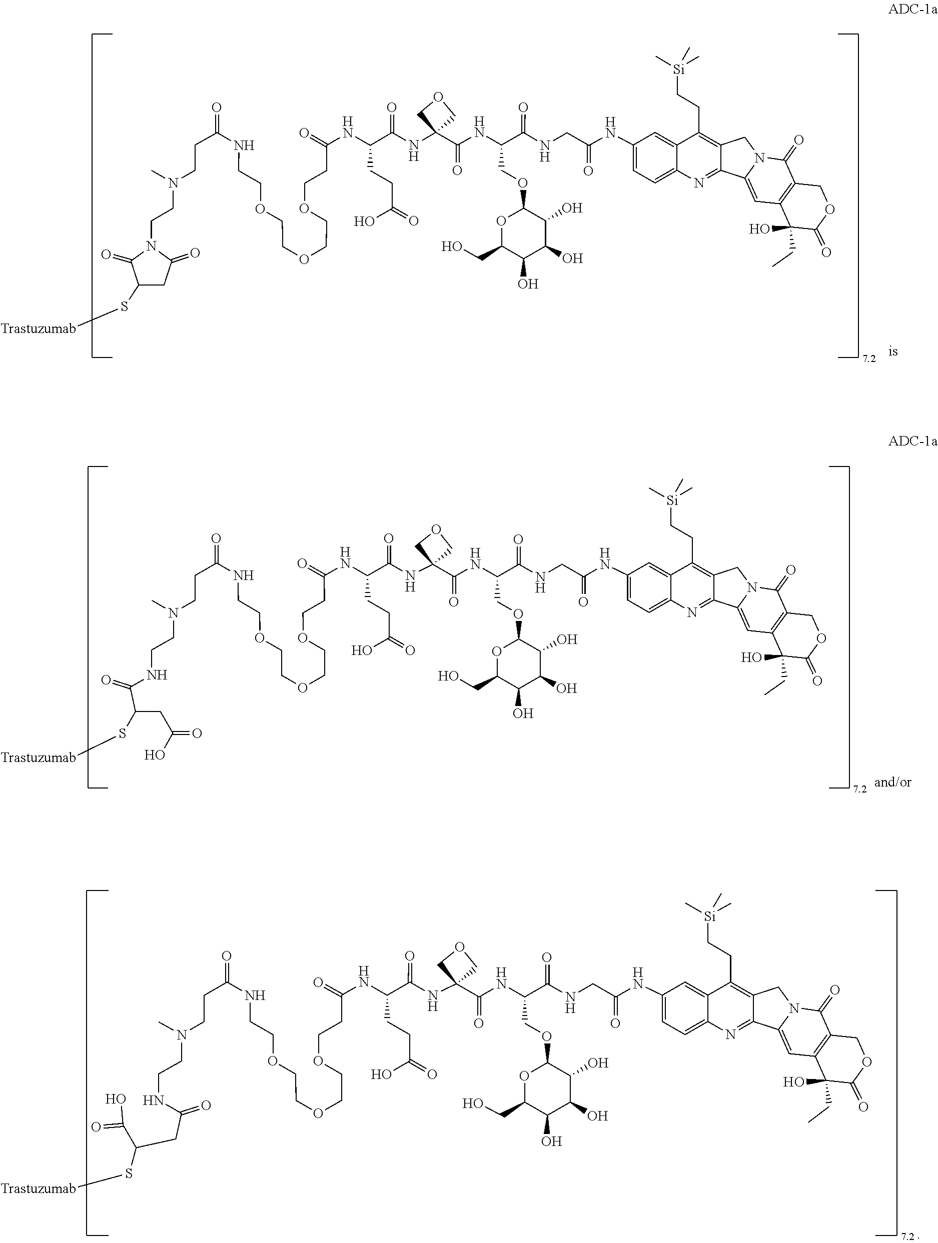

It can be understood by those skilled in the art that the position 1 of G^{T} is connected to the sulfhydryl group contained in Ab (such as antibody) itself after the disulfide bond is opened (for example, the disulfide bond can be opened by reducing the disulfide bond by reducing agent TCEP to generate sulfhydryl group —SH), that is, —S— between L₁ and Ab is not an additional sulfur atom externally attached. For example, in

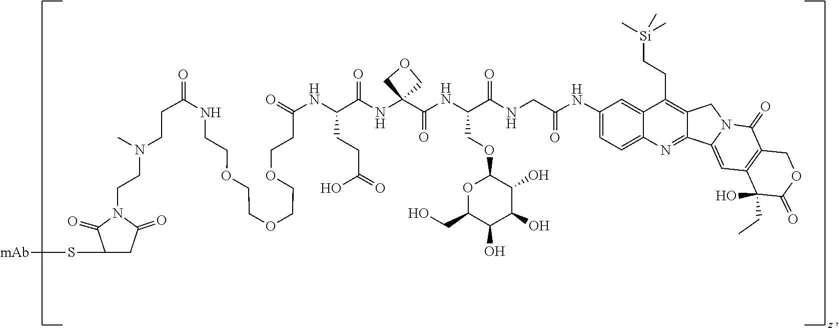

—S— is not an additional sulfur atom externally attached, but —S— formed after the sulfhydryl group contained in the Ab itself after opening the disulfide bond is connected to the position 1 of G^{T} such as

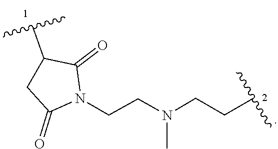 [SMILES: *CCN(C)CCN1C(=O)CC(*)C1=O]

The present disclosure provides a compound of formula III or a pharmaceutically acceptable salt thereof:

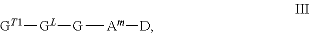

G^{T1} is a group connected to an antibody or an antigen-binding fragment thereof,
G^{L}, G, A^{m} and D are as described in any of the previous embodiments.

In a certain embodiment, G^{T1} is

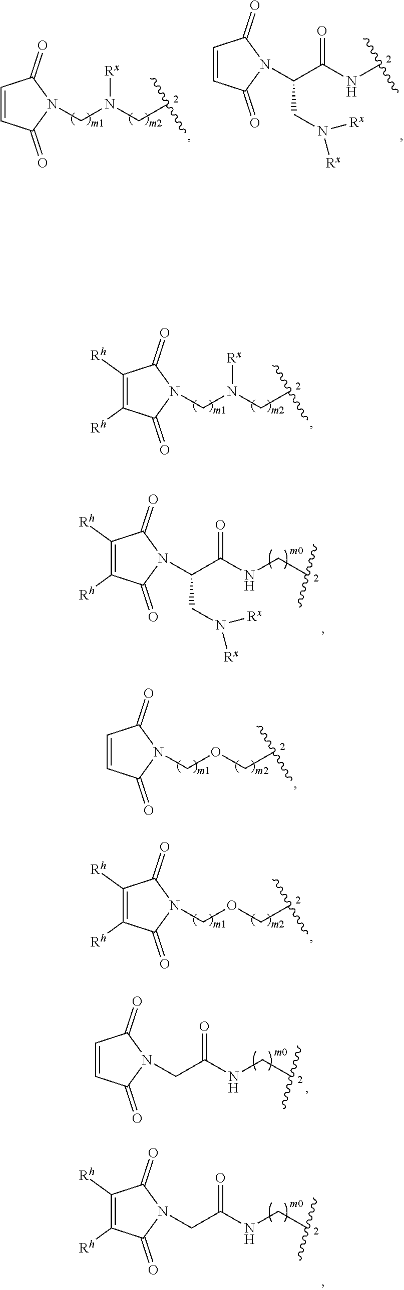

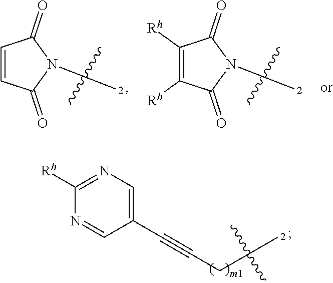

R^{h} is is a leaving group;
m0, m1, m2 and R^{x} are as described in any of the embodiments of the present disclosure.

In a certain embodiment, R^{h} is

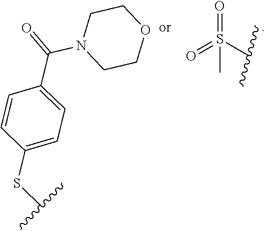

In a certain embodiment, G^{T1} is

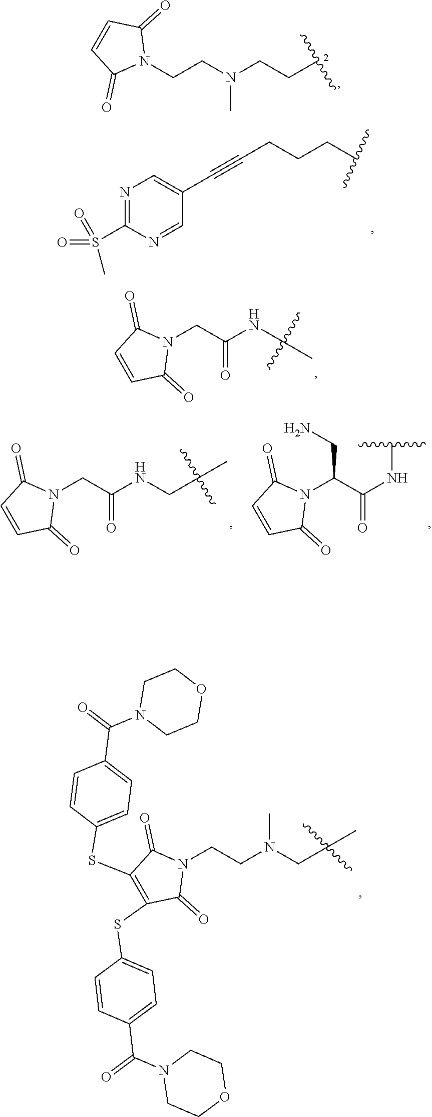

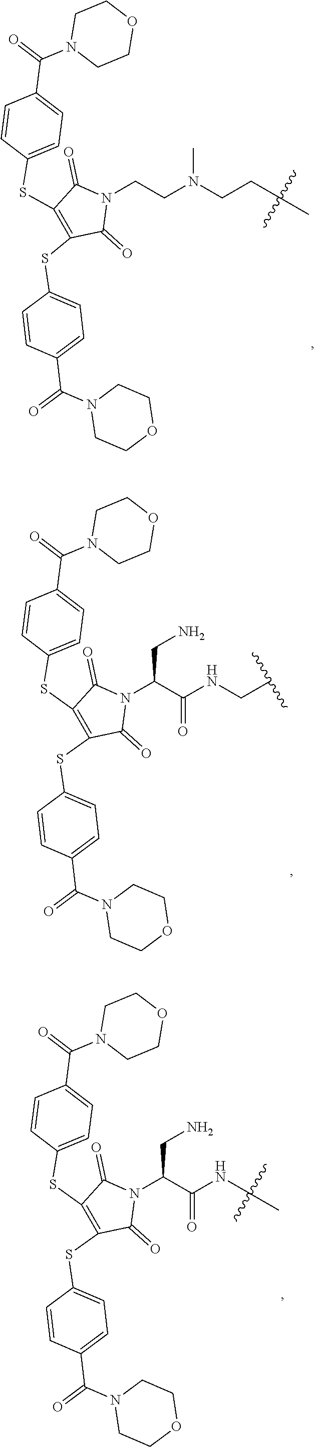

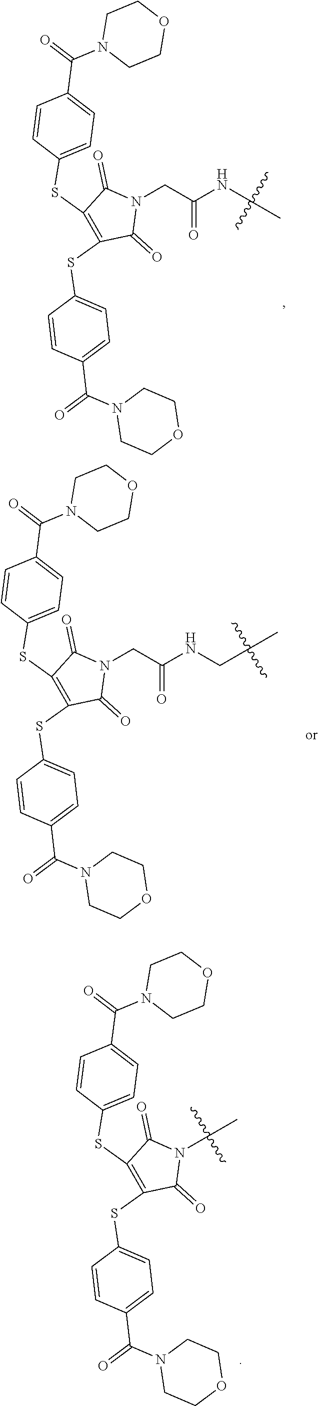

In a certain embodiment, the compound of formula III is any one of the following compounds:

| Com- | |
|---|---|
| pound | |
| number | Structural formula of linker-payload |
| L3P1 | 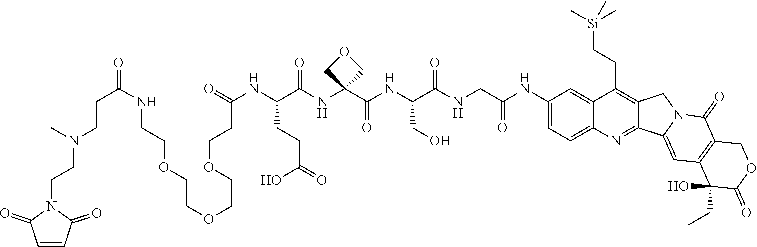 |
| L3aP1 | 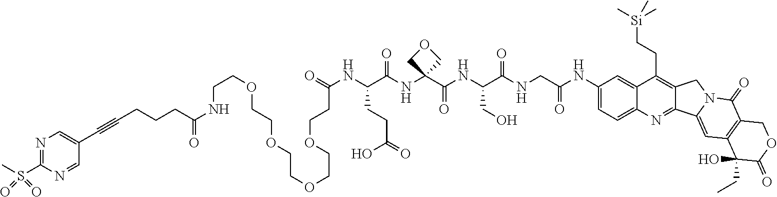 |
| L3bP1 | 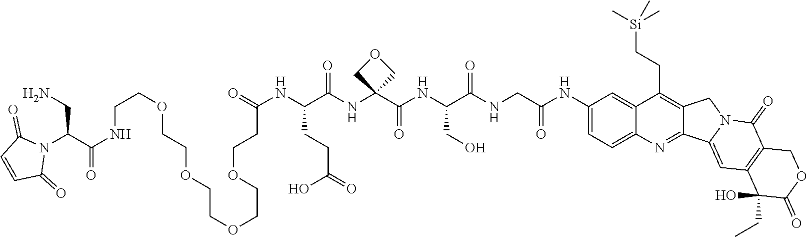 |
| L3cP1 | 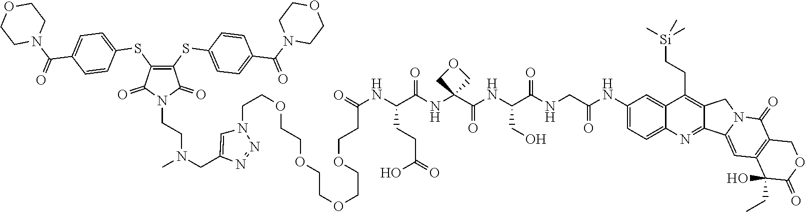 |
| L3dP1 | 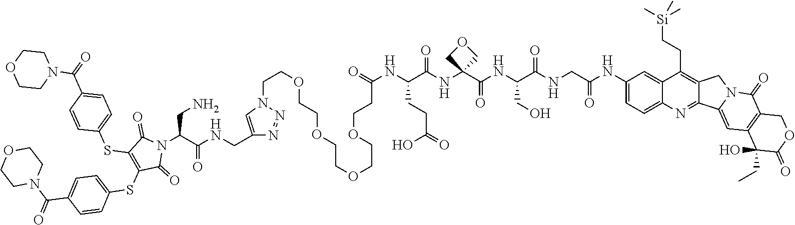 |
| L2P2 | 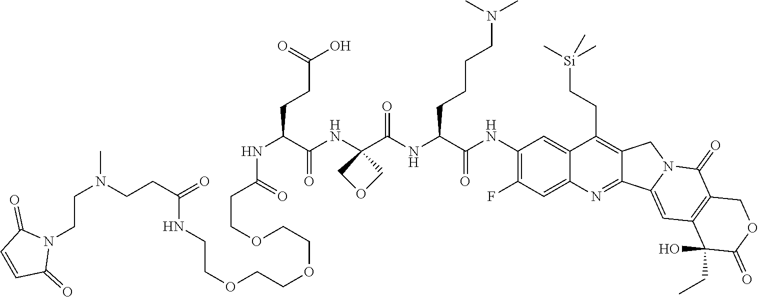 |
| L3P2 | 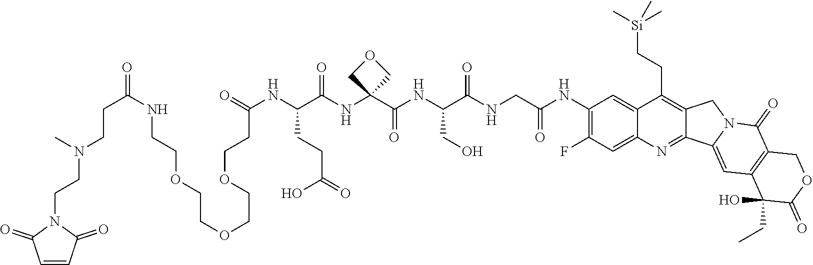 |
| L3aP2 | 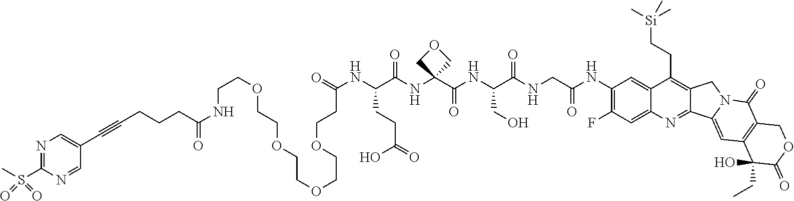 |
| L3bP2 | 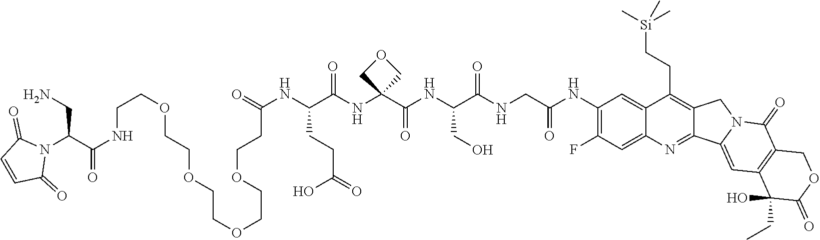 |
| L3cP2 | 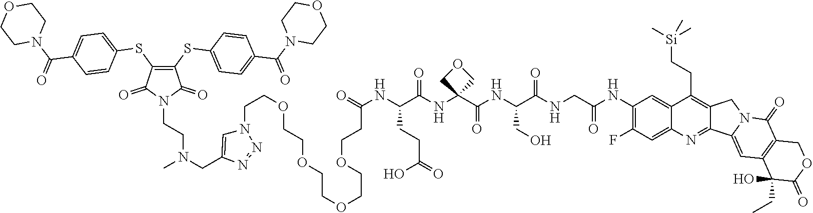 |
| L3dP2 | 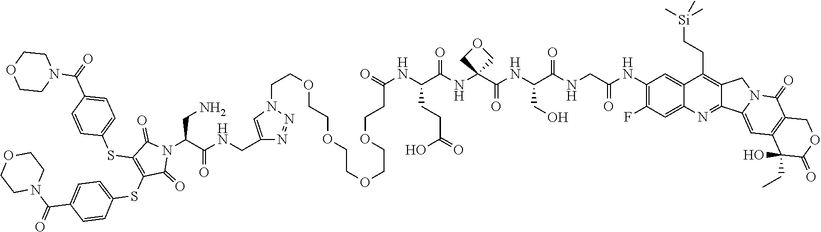 |
| L7P1 | 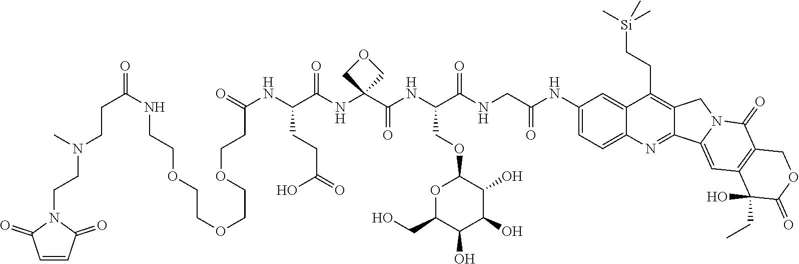 |
| L7P2 | 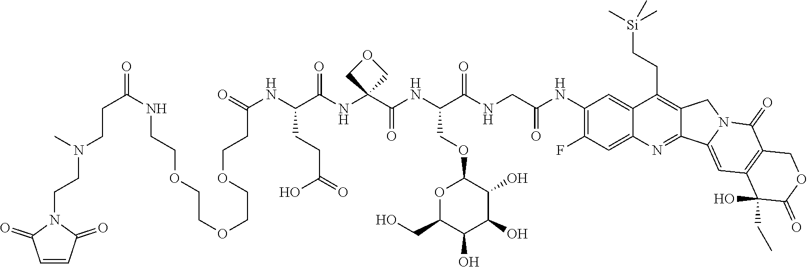 |
| L8rP1 | 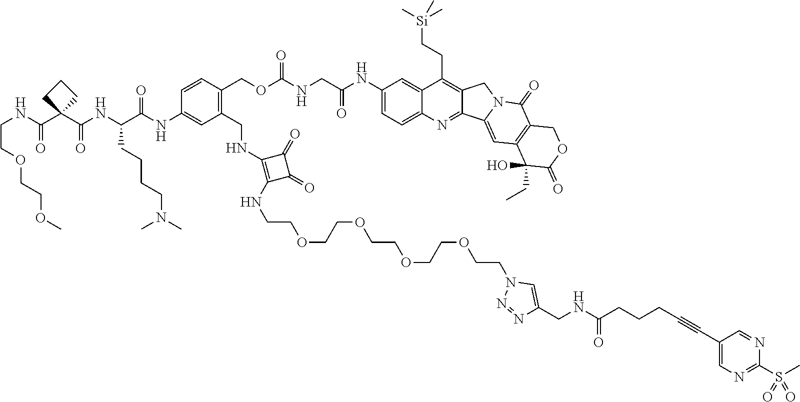 |
| L8rP2 | 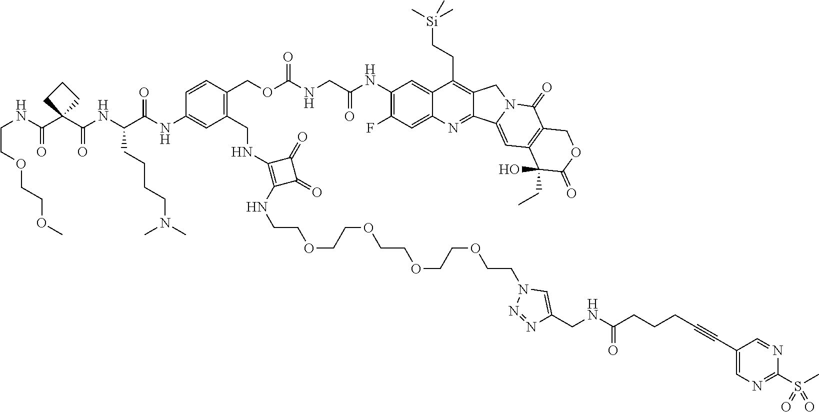 |
| L18aP2 | 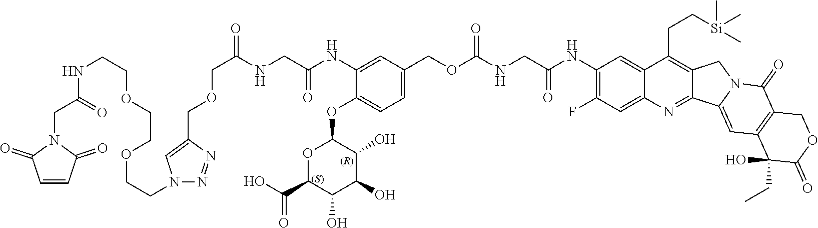 |
| L8rQP2 | 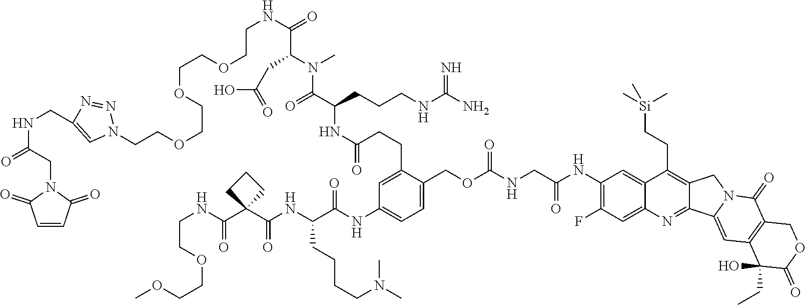 |
| L8rRP2 | 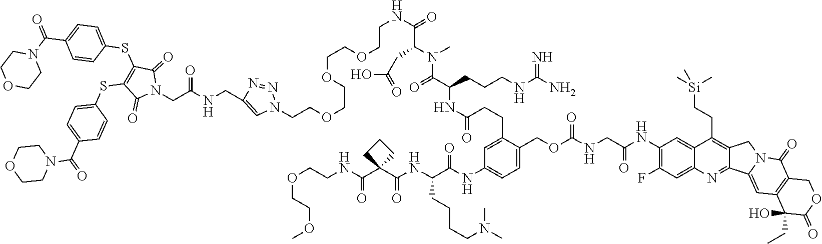 |
| L8rSP2 | 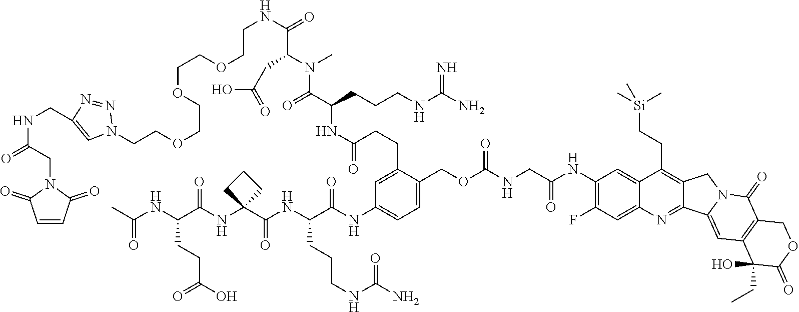 |
| L8rTP2 | 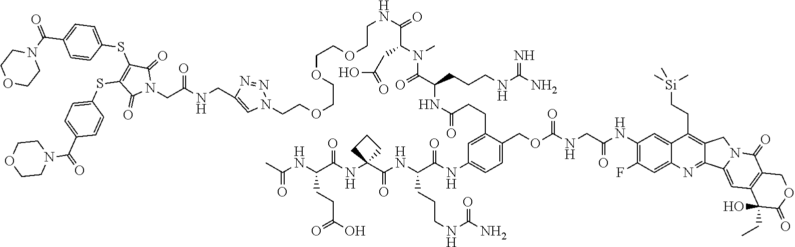 |

The present disclosure further provides a compound of formula I or a pharmaceutically acceptable salt thereof,

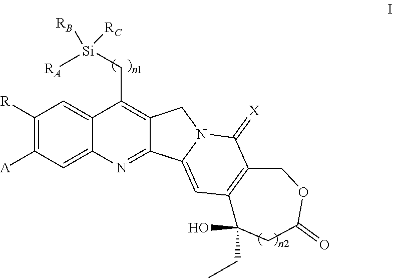

wherein, n1 is 1, 2 or 3;
R is amino or hydroxyl;
A is halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl; the 5- to 10-membered heteroaryl and 3- to 6-membered heterocyclyl each comprise one, two or three types of heteroatoms selected from N, O and S, and the number of the heteroatoms is independently 1, 2 or 3;

R^{A}, R^{B}, R^{C} and n2 are as described in any of the embodiments of the present disclosure.

In a certain embodiment, the compound of formula I is

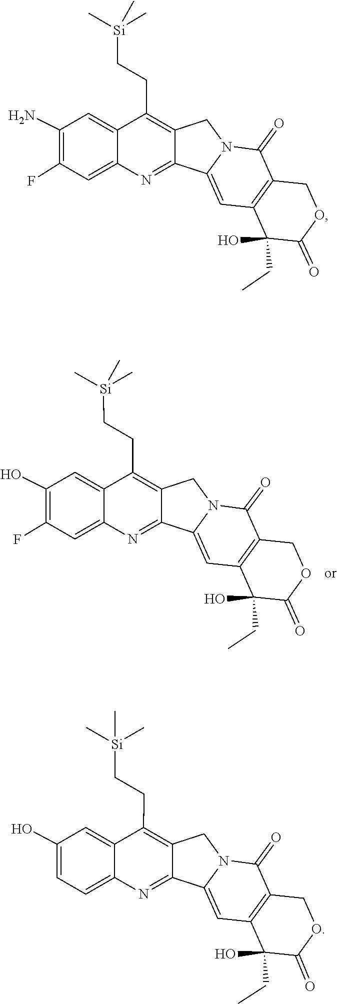 [SMILES: CC[C@@]1(O)C(=O)OCc2c1cc1n(c2=O)Cc2c-1nc1cc(F)c(N)cc1c2CC[Si](C)(C)C ; CC[C@@]1(O)C(=O)OCc2c1cc1n(c2=O)Cc2c-1nc1cc(F)c(O)cc1c2CC[Si](C)(C)C ; CC[C@@]1(O)C(=O)OCc2c1cc1n(c2=O)Cc2c-1nc1ccc(O)cc1c2CC[Si](C)(C)C]

The present disclosure provides a pharmaceutical composition, comprising:
(1) (a therapeutically effective amount of) the compound of formula I, II or III or the pharmaceutically acceptable salt thereof according to any one of the embodiments of the present disclosure, and
(2) a pharmaceutical excipient.

The present disclosure provides a use of the compound of formula I, II or III or the pharmaceutically acceptable salt thereof of any one of the embodiments of the present disclosure or the above pharmaceutical composition in the manufacture of a medicament for preventing and/or treating cancer, wherein the cancer may be ovarian cancer, breast ductal carcinoma, breast cancer, pancreatic adenocarcinoma (such as metastatic pancreatic adenocarcinoma) or pancreatic cancer.

The present disclosure provides a use of the drug conjugate of formula I, II or III or the pharmaceutically acceptable salt thereof of any one of the embodiments of the present disclosure or the above pharmaceutical composition in the manufacture of a medicament for preventing and/or treating a disease related to an antibody target (such as cancer).

In a certain embodiment, the antibody target is selected from one or more of Her2, folate receptor α, MUC1 and human IgG1, for example, the antibody target is Her2, MUC1, or Her2 and MUC1, for another example, the antibody target is Her2 and MUC1.

In a certain embodiment, the disease related to Her2, MUC1, or Her2 and MUC1 targets is cancer, such as ovarian cancer, breast ductal carcinoma, breast cancer, pancreatic adenocarcinoma (such as metastatic pancreatic adenocarcinoma), pancreatic cancer or gastric cancer.

In the present disclosure, the disease related to the antibody target refers to a disease expressing the target antigen of the antibody, for example, the target antigen of the Trastuzumab antibody or a variant thereof is Her2;
the target antigen of the v30384 antibody or a variant thereof is folate receptor α;
the target antigens of the FG1 antibody or a variant thereof, the Gatipotuzumab antibody or a variant thereof, and the 168D antibody or a variant thereof are MUC1;
the target antigens of the FG1-Tsc antibody or a variant thereof, the FG1-Tsc Thiomab antibody or a variant thereof, the Gati-Tsc antibody or a variant thereof, and the 186D-Tsc antibody or a variant thereof are Her2 and/or MUC1;
the Her2-expressing cancer is ovarian cancer, breast cancer, breast adenocarcinoma or gastric cancer;
the MUC1-expressing cancer is ovarian cancer or pancreatic adenocarcinoma.

### Definition of Terms

The term “ratio of drug to antibody or antigen-binding fragment thereof” or “DAR” refers to the number of drugs, e.g., camptothecin drugs attached to an antibody of ADC. The DAR of ADC can range from 1 to 8, but higher loadings (e.g., 20) are also possible depending on the number of attachment sites on the antibody. The term DAR may be used when referring to the number of drugs loaded onto a single antibody, or alternatively, when referring to the average or mean DAR of a group of ADCs.

In the present disclosure, the term “monoclonal antibody” refers to an antibody from a population of substantially homogeneous antibodies, that is, the antibodies constituting the population are identical except for a small amount of natural mutations that may be present. Monoclonal antibodies have high specificity against one determinant (epitope) of an antigen, while polyclonal antibodies comprise different antibodies against different determinants (epitopes). In addition to specificity, the advantages of monoclonal antibodies are that they may not be contaminated by other antibodies during synthesis. The modifier “monoclonal” herein indicates that the antibody is characterized as derived from a substantially homogeneous population of antibodies, and is not to be construed as necessarily being prepared by a particular method.

The term “antigen-binding fragment” refers to a portion of an antibody, preferably an antigen-binding region or a variable region. Examples of antibody fragments include Fab, Fab′, F(ab′)₂, Fd, Fv, dAb and complementarity determining region fragments, diabodies, linear antibodies and single chain antibody molecules. As used herein, the term “antigen-binding fragment” refers to a partial fragment of an antibody having antigen-binding activity, wherein the fragment has complete or partial function of the antibody, including but not limited to single-chain Fv(scFv), Fab, Fab′, F(ab′)₂, disulfide-linked Fv(sdFv), Fv, di-scFv, etc. The term also includes Fab′, which is a monovalent fragment of the variable region of an antibody obtained by treating F(ab′)₂ under reducing conditions. However, the term is not limited to these molecules as long as the fragment has binding affinity to the antigen. In addition, these functional fragments include not only fragments obtained by treating full-length molecules of antibody proteins with appropriate enzymes, but also proteins produced in appropriate host cells using genetically modified antibody genes.

The term “variant” refers to an antibody that maintains at least the same antigen-binding function as its antibody. For example, a variant of a Trastuzumab antibody is an antibody that has at least the same antigen-binding function as the Trastuzumab antibody.

The term “bispecific antibody”, also known as “bifunctional antibody conjugate”, refers to a conjugate formed by a first antibody (fragment) and a second antibody (fragment) through a coupling arm, and the conjugate retains the activity of the respective antibody and thus has bifunctionality and bispecificity.

In the present disclosure, the term “amino acid” refers to a natural amino acid and a non-natural amino acid, and conventional amino acids are written according to conventional usage. Non-natural amino acids refer to amino acids synthesized by artificial methods.

The term “non-natural amino acid” has the following structure:

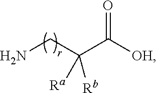

wherein r is selected from 0, 1, 2, 3, 4 and 5; wherein R^{a} and R^{b} are each independently selected from H, C₁₋₆ alkyl, —(CH₂)ₙ—R^{f1}, R^{f1} is —OH, —COOH, —NH₂, —NR^{ee-1}R^{ee-2},

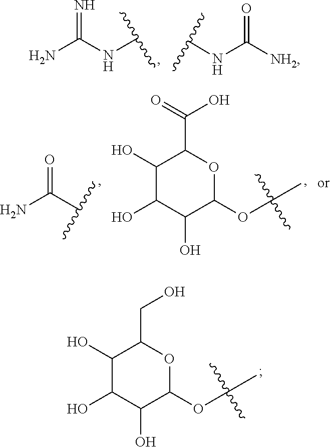

R^{ee-1} and R^{ee-2} are independently H or C₁₋₆ alkyl.

It can be understood by those skilled in the art that, according to the convention used in the art, “ [symbol] ” used in the structural formula of the group described in the present disclosure means that the corresponding group is connected to other fragments and groups in the compound through this site. “-” at the end of a group means that the group is attached to the rest of the molecule through that site, e.g., —OH refers to a hydroxyl group.

The term “optionally substituted or optionally substituted by a substituent” means that it can be substituted or unsubstituted, and unless otherwise specified, the type and number of substituents can be optional on the basis that it can be achieved chemically.

The term “optionally comprising” means either comprising or not comprising.

The term “pharmaceutically acceptable” refers to salts, solvents, excipients, and the like that are generally non-toxic, safe, and suitable for patient use. The “patient” is preferably a mammal, more preferably a human.

The term “pharmaceutically acceptable salt” refers to a salt prepared from a compound of the present disclosure and a relatively non-toxic pharmaceutically acceptable acid or base.

The term “pharmaceutical excipient” refers to an excipient and an additive used in the production and formulation of medicines, and is all substances contained in pharmaceutical preparations except active ingredients.

The term “treatment” refers to therapeutic therapy or alleviation measures. When referring to a specific condition, treatment refers to: (1) alleviating the disease or one or more than one biological manifestation of the condition, (2) interfering with (a) one or more than one point in the biological cascade that leads to or causes the condition or (b) one or more than one biological manifestation of the condition, (3) ameliorating one or more than one symptom, effect or side effect associated with the condition, or one or more than one symptom, effect or side effect associated with the condition or the treatment thereof, or (4) slowing the progression of the condition or one or more than one biological manifestation of the condition. “Treatment” may also refer to prolonging survival compared to a desired survival without treatment.

The term “prevention” refers to a reduced risk of acquiring or developing a disease or disorder.

The term “therapeutically effective amount” refers to the amount of the compound that, when administered to a patient, is sufficient to effectively treat the disease or condition described herein. The term “therapeutically effective amount” will vary based on the compound, the disease and its severity, as well as the age of the patient to be treated, but can be adjusted as needed by those skilled in the art.

The term “patient” refers to any animal that will receive, or has already received, administration of the compound or the composition according to examples of the present disclosure, with mammals being preferred and humans being most preferred. The term “mammal” includes any mammal. Examples of mammals include, but are not limited to, cattle, horses, sheep, pigs, cats, dogs, mice, rats, rabbits, guinea pigs, monkeys, humans, etc., with humans being most preferred.

Unless otherwise specified, the description “be independently . . . ” used in the present disclosure should be understood in a broad sense, which means that the described individuals are independent of each other, and may independently be the same or different specific groups. More specifically, the description “be independently . . . ” may mean that in different groups, specific options expressed by the same symbols do not affect each other; or may mean that in the same group, specific options expressed by the same symbols do not affect each other.

As used herein, a numerical range defined in a substituent such as 0-10, 1-6, 1-3, etc. indicates an integer or decimal within that range, for example, 1-6 is 1, 2, 3, 4, 5, 6.

If a linking group is represented as “absent (i.e., when the number is 0) or a single bond”, the structures on both sides of the linking group are directly connected by a single bond, for example, -A-B—C—; when B is a single bond or absent, -A-B—C— is -A-C—.

In the present disclosure, any combination of variables is allowed only if such combination results in a stable compound.

When any variable (for example, R^{1-a}) appears multiple times in the definition of the compound, the definition of each position of the variable is independent of the definitions of the remaining positions, and their meanings are independent of each other and do not affect each other. Therefore, if a certain group is substituted by 1, 2 or 3 R^{1-a} groups, that is, the group may be substituted by at most 3 R^{1-a}, wherein the definition of a certain position R^{1-a} and the definition of other positions R^{1-a} are independent of each other. In addition, combinations of substituents and/or variables are allowed only when the combination produces stable compounds.

When no substituents are expressly specified in the listed groups, such groups are meant to be unsubstituted. For example, when “C₁₋₆ alkyl” is not limited to “substituted or unsubstituted”, it only refers to “C₁₋₆ alkyl” itself or “unsubstituted C₁₋₆ alkyl”.

The term “more than one” means 2, 3, 4 or 5, preferably 2 or 3.

The term “halogen” refers to fluorine, chlorine, bromine or iodine.

The term “alkyl” refers to a linear or branched alkyl group having the specified number of carbon atoms (for example, C₁₋₆ alkyl), preferably C₁₋₄ alkyl. Alkyl groups include, but are not limited to, methyl, ethyl, n-propyl, isopropyl, n-butyl, tert-butyl, isobutyl, sec-butyl, n-pentyl and n-hexyl.

The term “alkoxy” refers to —O-alkyl, and the alkyl is defined as above. Alkoxy groups include, but are not limited to, methoxy, ethoxy, n-propoxy, isopropoxy, n-butoxy, tert-butoxy, isobutoxy and sec-butoxy.

The term “alkenyl” refers to an alkyl group as defined above consisting of at least two carbon atoms and at least one carbon-carbon double bond, and “C₂₋₆ alkenyl” refers to a linear or branched alkenyl group containing 2-6 carbon atoms, including but not limited to vinyl, 1-propenyl, 2-propenyl, 1-, 2- or 3-butenyl, etc., preferably “C₂₋₄ alkenyl”. In some embodiments, the alkenyl is C₂, C₃, C₄, C₅, C₆ alkenyl.

The term “alkynyl” refers to an alkyl group as defined above consisting of at least two carbon atoms and at least one carbon-carbon triple bond, and “C₂₋₆ alkynyl” refers to a linear or branched alkynyl group containing 2-6 carbon atoms, including but not limited to ethynyl, 1-propynyl, 2-propynyl, 1-, 2- or 3-butynyl, preferably “C₂₋₄ alkynyl”. In some embodiments, the alkynyl is C₂, C₃, C₄, C₅, C₆ alkynyl.

The term “aryl” refers to an aromatic group consisting of carbon atoms that satisfies the 4n+2 rule of conjugated hydrocarbon ring system, each ring being aromatic. In a certain embodiment, “aryl” refers to an aromatic group having 6 to 10 carbon atoms. Examples of aryl groups include, but are not limited to, phenyl, naphthyl, etc.

The term “heteroaryl” refers to a conjugated ring system having carbon atoms and 1 to 3 heteroatoms selected from nitrogen, oxygen and sulfur in the ring and being aromatic, each ring being aromatic. Unless otherwise specifically indicated in the specification, the heteroaryl may be a monocyclic, bicyclic, tricyclic, or more cyclic ring system. A 5- to 10-membered heteroaryl containing 1, 2 or 3 heteroatoms selected from N, O and S is preferred, and a 5- to 6-membered heteroaryl containing 1, 2 or 3 heteroatoms selected from N, O and S is more preferred.

The term “cycloalkyl” refers to a saturated monocyclic or polycyclic (bicyclic fused ring, bicyclic spiro ring, etc.) carbocyclic substituent with a specified number of ring carbon atoms (e.g., C₃₋₆), and it can be connected to the rest of the molecule through any suitable carbon atom by a single bond. Examples of cycloalkyl groups include, but are not limited to, cyclopropyl, cyclobutyl, cyclopentyl and cyclohexyl.

The term “heterocyclyl” refers to a saturated or partially saturated non-aromatic cyclic group having a specified number of ring atoms (e.g., 3- to 6-membered), a specified number of heteroatom groups (e.g., 1, 2 or 3), and a specified heteroatom group (e.g., 1, 2 or 3 types of N, O and S). In a certain embodiment of the present disclosure, heterocyclyl includes “saturated heterocyclyl (defined as heterocycloalkyl)” and “partially saturated heterocyclyl (such as heterocycloalkenyl)”, and heterocycloalkenyl refers to a stable partially saturated monocyclic or polycyclic (such as bicyclic, tricyclic or more cyclic) non-aromatic cyclic group having at least one double bond consisting of carbon atoms and 1, 2 or 3 heteroatoms selected from N, O and S.

The term “heterocycloalkyl” refers to a cyclic group having the specified number of ring atoms (e.g., 3- to 6-membered), the specified number of heteroatoms (e.g., 1, 2 or 3), and the specified heteroatom types (one or more types of N, O and S), which is a monocyclic ring, fused ring, bridged ring or spiro ring, and each ring is saturated.

### BENEFICIAL EFFECTS OF THE PRESENT DISCLOSURE

The conjugate provided by the present disclosure has good anti-tumor cell activity, and ADC and payload molecules can more effectively kill tumor cells, especially have good inhibitory effects on ovarian cancer, breast ductal carcinoma, breast cancer, pancreatic adenocarcinoma or pancreatic cancer. In summary, the payload molecule and ADC of the present disclosure have good clinical application prospects.

### DETAILED DESCRIPTION OF THE PREFERRED EMBODIMENT

The present disclosure will be further described below with reference to examples, but the present disclosure is not therefore limited to the scope of the examples. Experimental methods without specific conditions in the following examples are selected according to conventional methods and conditions, or according to the commercial specification.

### Antibody Sequences for Preparing ADC

| SEQ | | |
|---|---|---|
| ID No. | Sequence Name | Amino acid sequence |
| 1 | Heavy chain for | EVQLVESGGGLVQPGGSLRLSCAASGFNIKDTYIHW |
| | Tsc | VRQAPGKGLEWVARIYPTNGYTRYADSVKGRFTISA |
| | (Trastuzumab) | DTSKNTAYLQMNSLRAEDTAVYYCSRWGGDGFYAM |
| | | DYWGQGTLVTVSSDASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPELLGGPSVFLFPPKPKD |
| | | TLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| 2 | Light chain for | DIQMTQSPSSLSASVGDRVTITCRASQDVNTAVAWY |
| | Tsc | QQKPGKAPKLLIYSASFLYSGVPSRFSGSRSGTDFTLT |
| | (Trastuzumab) | ISSLQPEDFATYYCQQHYTTPPTFGQGTKVEIKQRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 3 | Heavy chain 1 for | EVQLLESGGGLVQPGGSLRLSCAVSGFSLSSYGVSW |
| | v30384 | VRQAPGKGLEWIGAVNSGGSAYYADSVKGRSTISRD |
| | | NSKNTVYLQMNSLRAEDTAVYYCARSGSGYPMDYL |
| | | AIWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCDKTHTCPPCPAPELLGGPSVFLFPPKPKDTL |
| | | MISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYVYPPSRDEL |
| | | TKNQVSLTCLVKGFYPSDIAVEWESNGQPENNYKTT |
| | | PPVLDSDGSFALVSKLTVDKSRWQQGNVFSCSVMHE |
| | | ALHNHYTQKSLSLSPG |
| 4 | Heavy chain 2 for | EVQLLESGGGLVQPGGSLRLSCAVSGFSLSSYGVSW |
| | v30384 | VRQAPGKGLEWIGAVNSGGSAYYADSVKGRSTISRD |
| | | NSKNTVYLQMNSLRAEDTAVYYCARSGSGYPMDYL |
| | | AIWGQGTLVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCDKTHTCPPCPAPELLGGPSVFLFPPKPKDTL |
| | | MISRTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYVLPPSRDEL |
| | | TKNQVSLLCLVKGFYPSDIAVEWESNGQPENNYLTW |
| | | PPVLDSDGSFFLYSKLTVDKSRWQQGNVFSCSVMHE |
| | | ALHNHYTQKSLSLSPG |
| 5 | Light chain for | DYQMTQSPSSVSASVGDRVTITCRASQSIGDWLAWY |
| | v30384 | QQKPGKAPKLLIYEASTLASGVPSRFSGSGSGTDFTL |
| | | TISSVQPEDAATYYCQQGYGRSNVDNIFGGGTKVEV |
| | | KRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| 6 | Heavy chain for | QVQLVQSGAEVKKPGASVKVSCKASGYTFSAYWIE |
| | FG1 | WVRQAPGKGLEWVGEILPGSNNSRYNEKFKGRVTV |
| | | TRDTSTNTAYMELSSLRSEDTAVYYCARSYDFAWFA |
| | | YWGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPELLGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSREEMTK |
| | | NQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGK |
| 7 | Light chain for | DIQMTQSPSSLSASVGDRVTITCKSSQSLLYSSNQKIY |
| | FG1 | LAWYQQKPGKAPKLLIYWASTRESGVPSRFSGSGSG |
| | | TDFTFTISSLQPEDIATYYCQQYYRYPRTFGQGTKVEI |
| | | KRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| 8 | Heavy chain for | EVQLVESGGGLVQPGGSMRLSCVASGFPFSNYWMN |
| | Gati | WVRQAPGKGLEWVGEIRLKSNNYTTHYAESVKGRF |
| | | TISRDDSKNSLYLQMNSLKTEDTAVYYCTRHYYFDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGK |
| 9 | Light chain for | DIVMTQSPLSNPVTPGEPASISCRSSKSLLHSNGITYFF |
| | Gati | WYLQKPGQSPQLLIYQMSNLASGVPDRFSGSGSGTD |
| | | FTLRISRVEAEDVGVYYCAQNLELPPTFGQGTKVEIK |
| | | RTVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |
| 10 | Heavy chain for | QMQLVQSEAELKKPGASVKVSCKASGYSFTGHYMH |
| | 186D | WVRQAPGQGLEWMGWIDPVTGGTKYAQNFQGWV |
| | | TMTRDTSIRTAYLELSRLRSDDTAMYYCAREVTGDR |
| | | GQFDKWGQGTLVTVASASTKGPSVFPLAPSSKSTSG |
| | | GTAALGCLVKDYFPEPVTVSWNSGALTSGVHTFPAV |
| | | LQSSGLYSLSSVVTVPSSSLGTQTYICNVNHKPSNTK |
| | | VDKKVEPKSCDKTHTCPPCPAPELLGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLPPS |
| | | REEMTKNQVSLTCLVKGFYPSDIAVEWESNGQPENN |
| | | YKTTPPVLDSDGSFFLYSKLTVDKSRWQQGNVFSCS |
| | | VMHEALHNHYTQKSLSLSPGK |
| 11 | Light chain for | QSVLTQPPSVSVAPGKTARITCGGNNIGSKSVHWYQ |
| | 186D | QKPGQAPALVIYYGSNRPSGIPERFSGSNSGNTATLTI |
| | | SRVEAGDEADYYCQVWDSSSDWVFGGGTKLTVLG |
| | | QPKAAPSVTLFPPSSEELQANKATLVCLISDFYPGAVT |
| | | VAWKADSSPVKAGVETTTPSKQSNNKYAASSYLSLT |
| | | PEQWKSHRSYSCQVTHEGSTVEKTVAPTECS |
| 12 | Heavy chain for | QVQLVQSGAEVKKPGASVKVSCKASGYTFSAYWIE |
| | FG1-Tsc | WVRQAPGKGLEWVGEILPGSNNSRYNEKFKGRVTV |
| | | TRDTSTNTAYMELSSLRSEDTAVYYCARSYDFAWFA |
| | | YWGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPELLGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSREEMTK |
| | | NQVSLTCLVKGFYPSDIAVEWESNGQPENNYKTTPP |
| | | VLDSDGSFFLYSKLTVDKSRWQQGNVFSCSVMHEAL |
| | | HNHYTQKSLSLSPGKGGGGSGGGGSEVQLVESGGG |
| | | LVQPGGSLRLSCAASGFNIKDTYIHWVRQAPGKCLE |
| | | WVARIYPTNGYTRYADSVKGRFTISADTSKNTAYLQ |
| | | MNSLRAEDTAVYYCSRWGGDGFYAMDYWGQGTLV |
| | | TVSSGGGGSGGGGSGGGGSGGGGSDIQMTQSPSSLS |
| | | ASVGDRVTITCRASQDVNTAVAWYQQKPGKAPKLLI |
| | | YSASFLYSGVPSRFSGSRSGTDFTLTISSLQPEDFATYY |
| | | CQQHYTTPPTFGCGTKVEIK |
| 13 | Light chain for | DIQMTQSPSSLSASVGDRVTITCKSSQSLLYSSNQKIY |
| | FG1-Tsc | LAWYQQKPGKAPKLLIYWASTRESGVPSRFSGSGSG |
| | | TDFTFTISSLQPEDIATYYCQQYYRYPRTFGQGTKVEI |
| | | KRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| 14 | Heavy chain for | EVQLVESGGGLVQPGGSMRLSCVASGFPFSNYWMN |
| | Gati-Tsc | WVRQAPGKGLEWVGEIRLKSNNYTTHYAESVKGRF |
| | | TISRDDSKNSLYLQMNSLKTEDTAVYYCTRHYYFDY |
| | | WGQGTLVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMISR |
| | | TPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSREEMTKN |
| | | QVSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGKGGGGSGGGGSEVQLVESGGGLV |
| | | QPGGSLRLSCAASGFNIKDTYIHWVRQAPGKCLEWV |
| | | ARIYPTNGYTRYADSVKGRFTISADTSKNTAYLQMN |
| | | SLRAEDTAVYYCSRWGGDGFYAMDYWGQGTLVTV |
| | | SSGGGGSGGGGSGGGGSGGGGSDIQMTQSPSSLSAS |
| | | VGDRVTITCRASQDVNTAVAWYQQKPGKAPKLLIYS |
| | | ASFLYSGVPSRFSGSRSGTDFTLTISSLQPEDFATYYC |
| | | QQHYTTPPTFGCGTKVEIK |
| 15 | Light chain for | DIVMTQSPLSNPVTPGEPASISCRSSKSLLHSNGITYFF |
| | Gati-Tsc | WYLQKPGQSPQLLIYQMSNLASGVPDRFSGSGSGTD |
| | | FTLRISRVEAEDVGVYYCAQNLELPPTFGQGTKVEIK |
| | | RTVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |
| 16 | Heavy chain for | QMQLVQSEAELKKPGASVKVSCKASGYSFTGHYMH |
| | 186D-Tsc | WVRQAPGQGLEWMGWIDPVTGGTKYAQNFQGWV |
| | | TMTRDTSIRTAYLELSRLRSDDTAMYYCAREVTGDR |
| | | GQFDKWGQGTLVTVASASTKGPSVFPLAPSSKSTSG |
| | | GTAALGCLVKDYFPEPVTVSWNSGALTSGVHTFPAV |
| | | LQSSGLYSLSSVVTVPSSSLGTQTYICNVNHKPSNTK |
| | | VDKKVEPKSCDKTHTCPPCPAPELLGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLPPS |
| | | REEMTKNQVSLTCLVKGFYPSDIAVEWESNGQPENN |
| | | YKTTPPVLDSDGSFFLYSKLTVDKSRWQQGNVFSCS |
| | | VMHEALHNHYTQKSLSLSPGKGGGGSGGGGSEVQL |
| | | VESGGGLVQPGGSLRLSCAASGFNIKDTYIHWVRQA |
| | | PGKCLEWVARIYPTNGYTRYADSVKGRFTISADTSK |
| | | NTAYLQMNSLRAEDTAVYYCSRWGGDGFYAMDYW |
| | | GQGTLVTVSSGGGGSGGGGSGGGGSGGGGSDIQMT |
| | | QSPSSLSASVGDRVTITCRASQDVNTAVAWYQQKPG |
| | | KAPKLLIYSASFLYSGVPSRFSGSRSGTDFTLTISSL |
| | | QPEDFATYYCQQHYTTPPTFGCGTKVEIK |
| 17 | Light chain for | QSVLTQPPSVSVAPGKTARITCGGNNIGSKSVHWYQ |
| | 186D-Tsc | QKPGQAPALVIYYGSNRPSGIPERFSGSNSGNTATLTI |
| | | SRVEAGDEADYYCQVWDSSSDWVFGGGTKLTVLG |
| | | QPKAAPSVTLFPPSSEELQANKATLVCLISDFYPGAVT |
| | | VAWKADSSPVKAGVETTTPSKQSNNKYAASSYLSLT |
| | | PEQWKSHRSYSCQVTHEGSTVEKTVAPTECS |
| 18 | VH for DNP | QVQLVQSGAEVKKPGASVKVSCKASGYTFTSYGIS |
| | | WVRQAPGQGLEWMGWISAYNGNTNYAQKLQGRVT |
| | | MTTDTSTSTAYMELRSLRSDDTAVYYCARWDSSSWS |
| | | FDYWGQGTLVTVSS |
| 19 | VL for DNP | DIQMTQSPSSLSASVGDRVTITCRASQGISSWLAWYQ |
| | | QKPEKAPKSLIYAASSLQSGVPSRFSGSGSGTDFTLTI |
| | | SSLQPEDFATYYCQQYNSYPYTFGQGTKLEIKR |
| 20 | Heavy chain for | QVQLVQSGAEVKKPGASVKVSCKASGYTFSAYWIE |
| | FG1-Tsc thiomab | WVRQAPGKGLEWVGEILPGSNNSRYNEKFKGRVTV |
| | (HC-S375C) | TRDTSTNTAYMELSSLRSEDTAVYYCARSYDFAWFA |
| | | YWGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKKV |
| | | EPKSCDKTHTCPPCPAPELLGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSREEMTK |
| | | NQVSLTCLVKGFYPCDIAVEWESNGQPENNYKTTPP |
| | | VLDSDGSFFLYSKLTVDKSRWQQGNVFSCSVMHEAL |
| | | HNHYTQKSLSLSPGKGGGGSGGGGSEVQLVESGGG |
| | | LVQPGGSLRLSCAASGFNIKDTYIHWVRQAPGKCLE |
| | | WVARIYPTNGYTRYADSVKGRFTISADTSKNTAYLQ |
| | | MNSLRAEDTAVYYCSRWGGDGFYAMDYWGQGTLV |
| | | TVSSGGGGSGGGGSGGGGSGGGGSDIQMTQSPSSLS |
| | | ASVGDRVTITCRASQDVNTAVAWYQQKPGKAPKLLI |
| | | YSASFLYSGVPSRFSGSRSGTDFTLTISSLQPEDFATYY |
| | | CQQHYTTPPTFGCGTKVEIK |
| 21 | Light chain for | DIQMTQSPSSLSASVGDRVTITCKSSQSLLYSSNQKIY |
| | FG1-Tsc thiomab | LAWYQQKPGKAPKLLIYWASTRESGVPSRFSGSGSG |
| | (LC-V205C) | TDFTFTISSLQPEDIATYYCQQYYRYPRTFGQGTKVEI |
| | | KRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPCTKSFNRGEC |

Abbreviation list of amino acids

| | | |
|---|---|---|
| Alanine | Ala | A |
| Arginine | Arg | R |
| Asparagine | Asn | N |
| Aspartate | Asp | D |
| Aspartate or Asparagine | Asx | B |
| Cysteine | Cys | C |
| Glutamate | Glu | E |
| Glutamine | Gln | Q |
| Glutamate or Glutamine | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Abbreviation list of chemicals and instruments

| | |
|---|---|
| TLC | Thin layer chromatography |
| UV | Ultra violet |
| LC-MS | Liguid chromatography mass spectrometry |
| HPLC | High-performance liquid chromatography |
| RP | Reverse phase |
| NMR | Nuclear magnetic resonance |
| DBU | 1,8-diazabicyclo[5.4.0]undec-7-ene |
| DMF | Dimethyl formamide |
| DCM | dichloromethane |
| TBS | Tert-bulty dimethyl silane |
| MS | Molecular sieves |
| TFA | Trifluoro acetic acid |
| DMSO | Dimethyl sulfoxide |
| DMSO-d6 | Deuterated DMSO |
| DIPEA/DIEA | Diisopropyl ethylamine |
| ESI | Electrospray ionization |
| THF | Tetrahydrofuran |
| TEA | triethylamine |
| r.t. | Room temperature |
| RT | Retention time |
| HATU | 2-(7-Azabenzotriazol-1-yl)-N,N,N′,N′- |
| | tetramethyluronium hexafluorophosphate |
| HBTU | O-Benzotriazolyl-N,N,N′,N′-tetramethyluronium |
| | hexafluorophosphate |
| PPTS | p-Toluenesulfonicacid, pyridineadduct |
| X-phos | 2-Dicyclohexylphosphino-2′,4′,6′-triisopropylbiphenyl |
| NMI | 1-methylimidazole |
| TCFH | Chloro-N,N,N′,N′-tetramethylformamidinium |
| | hexafluorophosphate |
| Fmoc | fluorenylmethoxycarbonyl |
| Boc | tert-butoxycarbonyl |

### Preparation Example of Intermediate: Synthesis of Linker Intermediate

### Intermediate HPH-L01: tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

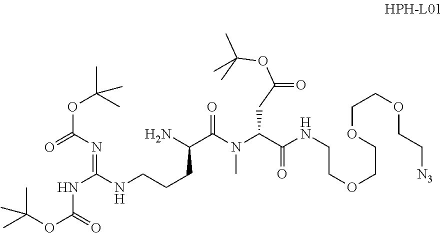 [SMILES: CN(C(=O)[C@H](N)CCCN/C(=N/C(=O)OC(C)(C)C)NC(=O)OC(C)(C)C)[C@H](CC(=O)OC(C)(C)C)C(=O)NCCOCCOCCOCCN=[N+]=[N-]]

### Intermediate HPH-L01-a: tert-butyl (R)-14-((((9H-fluoren-9-yl)methoxy)carbonyl)(methyl)amino)-1-azido-13-oxo-3,6,9-trioxa-12-azahexadecan-16-oate

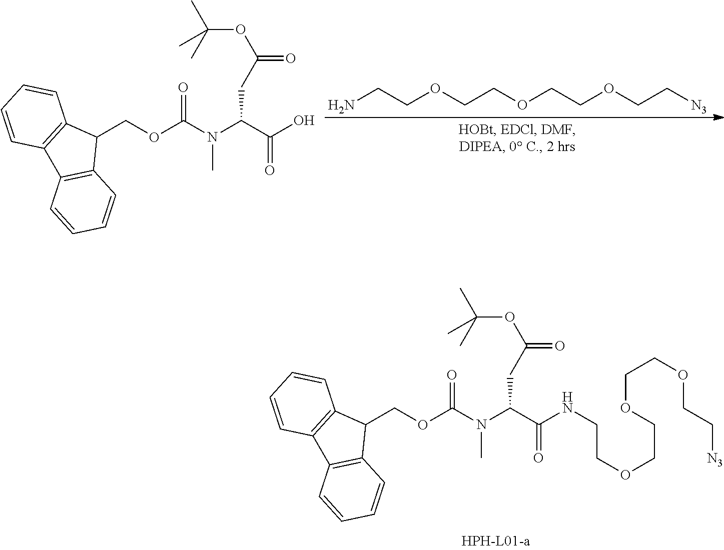 [SMILES: CN(C(=O)OCC1c2ccccc2-c2ccccc21)[C@H](CC(=O)OC(C)(C)C)C(=O)O ; [N-]=[N+]=NCCOCCOCCOCCN ; CN(C(=O)OCC1c2ccccc2-c2ccccc21)[C@H](CC(=O)OC(C)(C)C)C(=O)NCCOCCOCCOCCN=[N+]=[N-]]

(R)-2-((((9H-Fluoren-9-yl)methoxy)carbonyl)(methyl)amino)-4-(tert-butoxy)-4-oxobutanoic acid (CAS RN: 1799443-40-5; 20 g, 47.01 mmol) was dissolved in DMF (CAS RN: 68-12-2; 150 mL) at 0° C. Then HOBt (CAS RN: 2592-95-2; 9.53 g, 70.51 mmol) and EDCI (CAS RN: 25952-53-8, 13.4 g, 70.51 mmol) were added to the reaction mixture at 0° C. The reaction mixture was stirred at 0° C. for 2 hours before the addition of amino-PEG3-azide (CAS RN: 134179-38-7; 1 g, 50.4 mmol) and DIPEA (CAS RN: 7087-68-5; 7.74 g, 60.0 mmol). The reaction mixture was warmed to room temperature and stirred for another 16 hours. The reaction mixture was quenched by addition of 1 liter of distilled water. Ethyl acetate (CAS RN: 141-78-6) was used to extract the product from the reaction mixture (3×300 ml). The organic phase was combined and washed with saturated brine for 5 times (5×500 ml). The organic phase was dried over anhydrous sodium sulfate, and the organic solvent was reduced in vacuo to afford the reaction residue. The residue was purified over silica chromatography (DCM:methanol=10:1) to afford the desirable intermediate HPH-L01-a (tert-butyl (R)-14-((((9H-fluoren-9-yl)methoxy)carbonyl)(methyl)amino)-1-azido-13-oxo-3,6,9-trioxa-12-azahexadecan-16-oate; 28 g, 44.71 mmol, 95.09% in yield) as a colorless oil.

LC-MS (m/z, C₃₂H₄₃N₅O₈)=626.3 [M+H]⁺.

¹H NMR (400 MHz, DMSO-d6) δ 8.00 (t, J=8.0 Hz, 1H), 7.97-7.91 (m, 2H), 7.69-7.65 (m, 2H), 7.49-7.44 (m, 2H), 7.37-7.31 (m, 2H), 4.90-4.86 (m, 1H), 4.39-4.25 (m, 3H), 3.64-3.51 (m, 9H), 3.54-3.38 (m, 4H), 3.30-3.17 (m, 2H), 2.85-2.74 (m, 5H), 2.46-2.37 (m, 1H), 1.35 (s, 9H).

### Intermediate HPH-L01-b: tert-butyl (R)-1-azido-14-(methylamino)-13-oxo-3,6,9-trioxa-12-azahexadecan-16-oate

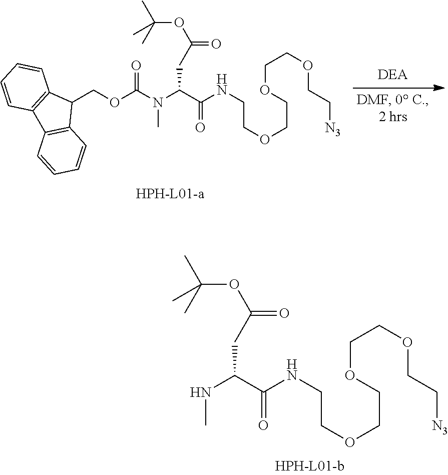 [SMILES: CN(C(=O)OCC1c2ccccc2-c2ccccc21)[C@H](CC(=O)OC(C)(C)C)C(=O)NCCOCCOCCOCCN=[N+]=[N-] ; CN[C@H](CC(=O)OC(C)(C)C)C(=O)NCCOCCOCCOCCN=[N+]=[N-]]

Intermediate HPH-L01-a (tert-butyl (R)-14-((((9H-fluoren-9-yl)methoxy)carbonyl)(methyl)amino)-1-azido-13-oxo-3,6,9-trioxa-12-azahexadecan-16-oate; 28 g, 44.71 mmol) was dissolved in DMF (CAS RN: 68-12-2; 150 mL) at room temperature. Then diethylamine (CAS RN: 109-89-7; 10.26 g, 223.5 mmol) was added to the reaction mixture, and the reaction mixture was stirred at 0° C. for 2 hours. Then 1-liter distilled water was pulled in to quench the reaction mixture. Ethyl acetate (CAS RN: 141-78-6) was used to extract the product from the reaction mixture (3×300 ml). The organic phase was combined and washed with saturated brine for 5 times (5×500 ml). The organic phase was dried over anhydrous sodium sulfate, and the organic solvent was reduced in vacuo to afford the reaction residue. The residue was purified over silica chromatography (DCM:methanol=10:1) to afford the desirable intermediate HPH-L01-b (tert-butyl (R)-1-azido-14-(methylamino)-13-oxo-3,6,9-trioxa-12-azahexadecan-16-oate; 18 g, 44.53 mmol, 99.60% in yield) as a yellow oil.

LC-MS (m/z, C₁₇H₃₃N₅O₆)=404.2 [M+H]⁺.

¹H NMR (400 MHz, DMSO-d6) δ 7.97 (t, J=8.0 Hz, 1H), 3.63-3.55 (m, 10H), 3.45-3.395 (m, 4H), 3.27-3.21 (m, 3H), 2.53-2.51 (m, 1H), 2.34-2.31 (m, 1H), 2.22 (s, 3H), 1.40 (s, 9H).

### Intermediate HPH-L01-c: tert-butyl (R)-14-((R)-2-((((9H-fluoren-9-yl)methoxy)carbonyl)amino)-5-((tert-butoxycarbonyl)amino)-N-methylpentanamido)-1-azido-13-oxo-3,6,9-trioxa-12-azahexadecan-16-oate

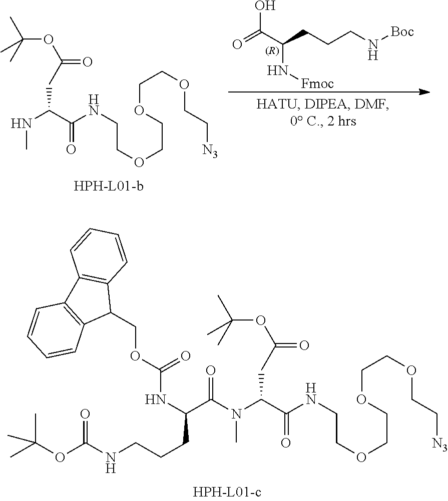

Intermediate HPH-L01-b (tert-butyl (R)-1-azido-14-(methylamino)-13-oxo-3,6,9-trioxa-12-azahexadecan-16-oate; 18 g, 44.53 mmol) was dissolved in DMF (CAS RN: 68-12-2; 210 mL) at 0° C. Then, Fmoc-D-Orn(Boc)-OH (CAS RN: 118476-89-4, 26.36 g, 58.00 mmol), HATU (CAS RN: 148893-10-1; 25.44 g, 66.91 mmol) and DIPEA (CAS RN: 7087-68-5; 11.53 g, 89.37 mmol) were added to the reaction mixture. The reaction mixture was warmed to room temperature and stirred for another 2 hours. The reaction mixture was quenched by addition of 1 liter of distilled water. Ethyl acetate (CAS RN: 141-78-6) was used to extract the product from the reaction mixture (3×300 ml). The organic phase was combined and washed with saturated brine for 5 times (5×500 ml). The organic phase was dried over anhydrous sodium sulfate, and the organic solvent was reduced in vacuo to afford the reaction residue. The residue was purified over silica chromatography (DCM:methanol=10:1) to afford the desirable intermediate HPH-L01-c (tert-butyl (R)-14-((R)-2-((((9H-fluoren-9-yl)methoxy)carbonyl)amino)-5-((tert-butoxycarbonyl)amino)-N-methylpentanamido)-1-azido-13-oxo-3,6,9-trioxa-12-azahexadecan-16-oate; 31 g, 36.91 mmol, 82.89% in yield) as a white solid.

LC-MS (m/z, C₄₂H₆₁N₇O₁₁)=740.3 [M−100]⁺.

### Intermediate HPH-L01-d: tert-butyl (R)-14-((R)-2-((((9H-fluoren-9-yl)methoxy)carbonyl)amino)-5-amino-N-methylpentanamido)-1-azido-13-oxo-3,6,9-trioxa-12-azahexadecan-16-oate

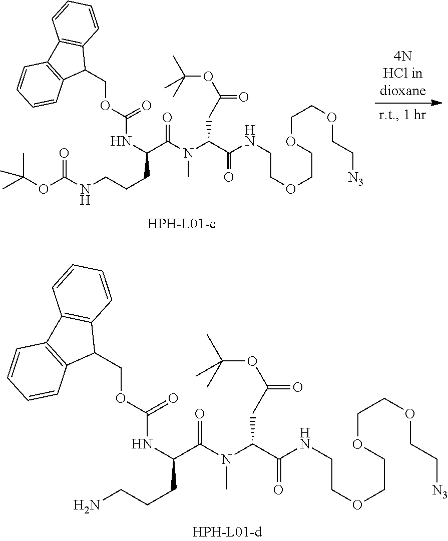 [SMILES: CN(C(=O)[C@@H](CCCNC(=O)OC(C)(C)C)NC(=O)OCC1c2ccccc2-c2ccccc21)[C@H](CC(=O)OC(C)(C)C)C(=O)NCCOCCOCCOCCN=[N+]=[N-] ; CN(C(=O)[C@@H](CCCN)NC(=O)OCC1c2ccccc2-c2ccccc21)[C@H](CC(=O)OC(C)(C)C)C(=O)NCCOCCOCCOCCN=[N+]=[N-]]

Intermediate HPH-L01-c (tert-butyl (R)-14-((R)-2-((((9H-fluoren-9-yl)methoxy)carbonyl)amino)-5-((tert-butoxycarbonyl)amino)-N-methylpentanamido)-1-azido-13-oxo-3,6,9-trioxa-12-azahexadecan-16-oate; 31 g, 36.91 mmol) was dissolved in ethyl acetate (CAS RN: 141-78-6; 210 mL). HCl in dioxane (4N; 70 mL) was added to the reaction mixture, and the reaction mixture was stirred for 1 hour at room temperature. The organic solvent in the reaction mixture was evaporated in vacuo to afford the desirable intermediate HPH-L01-d (tert-butyl (R)-14-((R)-2-((((9H-fluoren-9-yl)methoxy)carbonyl)amino)-5-amino-N-methylpentanamido)-1-azido-13-oxo-3,6,9-trioxa-12-azahexadecan-16-oate; 18 g, 24.31 mmol; 65.86% in yield) as a white solid.

LC-MS (m/z, C₃₇H₅₃N₇O₉)=740.4 [M+H]⁺.

### Intermediate HPH-L01-e: tert-butyl (11R,14R,Z)-11-((((9H-fluoren-9-yl)methoxy)carbonyl)amino)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

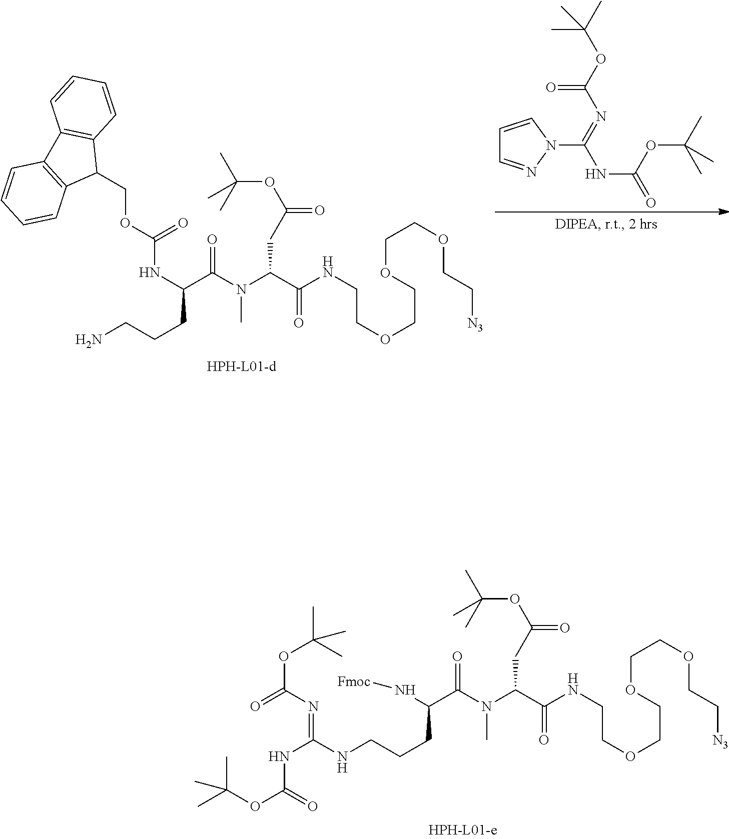

Intermediate HPH-L01-d (tert-butyl (R)-14-((R)-2-((((9H-fluoren-9-yl)methoxy)carbonyl)amino)-5-amino-N-methylpentanamido)-1-azido-13-oxo-3,6,9-trioxa-12-azahexadecan-16-oate; 27 g, 36.49 mmol) was dissolved in DMF (CAS RN: 68-12-2; 60 mL). The reaction mixture was added with N,N′-bis-Boc-1-guanylpyrazol (CAS RN: 152120-54-2, 13.59 g, 43.79 mmol) and DIPEA (CAS RN: 7087-68-5, 7.07 g, 54.80 mmol) at room temperature. The reaction mixture was stirred for another 2 hours at room temperature. The reaction mixture was quenched by addition of 1 liter of distilled water. Ethyl acetate (CAS RN: 141-78-6) was used to extract the product from the reaction mixture (3×300 ml). The organic phase was combined and washed with saturated brine for 5 times (5×500 ml). The organic phase was dried over anhydrous sodium sulfate, and the organic solvent was reduced in vacuo to afford the reaction residue. The residue was purified over silica chromatography (petroleum ether:ethyl acetate=10:1) to afford the desirable intermediate HPH-L01-e (tert-butyl (11R,14R,Z)-11-((((9H-fluoren-9-yl)methoxy)carbonyl)amino)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 14 g, 14.25 mmol, 39.05% in yield) as a white solid.

LC-MS (m/z, C₄₈H₇₁N₉O₁₃)=982.4 [M+H]⁺.

### Intermediate HPH-L01: tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

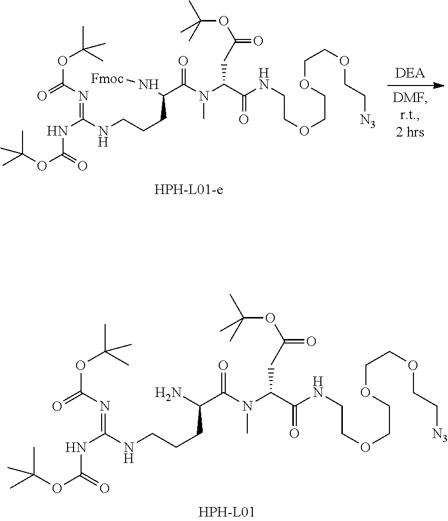

Intermediate HPH-L01-e (tert-butyl (11R,14R,Z)-11-((((9H-fluoren-9-yl)methoxy)carbonyl)amino)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 14 g, 14.25 mmol) was dissolved in DMF (CAS RN: 68-12-2; 60 mL) at room temperature. Then the reaction mixture was added with diethylamine (CAS RN: 109-89-7, 10.26 g, 223.5 mmol) and the reaction mixture was stirred for 2 hours at room temperature. The reaction mixture was quenched by addition of 1 liter of distilled water. Ethyl acetate (CAS RN: 141-78-6) was used to extract the product from the reaction mixture (3×300 ml). The organic phase was combined and washed with saturated brine for 5 times (5×500 ml). The organic phase was dried over anhydrous sodium sulfate, and the organic solvent was reduced in vacuo to afford the reaction residue. The residue was purified over silica chromatography (DCM:methanol=10:1) to afford the desirable intermediate HPH-L01 (tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 9 g, 11.84 mmol, 83.09% in yield) as a white solid.

LC-MS (m/z, C₃₃H₆₁N₉O₁₁)=760.4 [M+H]⁺.

### Intermediate HPH-L02: (S)-1-(2-acetamido-5-(tert-butoxy)-5-oxopentanamido)cyclobutane-1-carboxylic acid

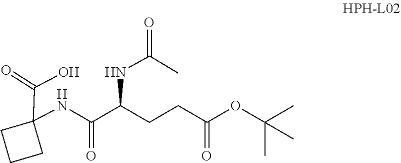 [SMILES: CC(=O)N[C@@H](CCC(=O)OC(C)(C)C)C(=O)NC1(C(=O)O)CCC1]

### Synthetic Method for Intermediate HPH-L02 (Solid-Phase Peptide Synthesis)

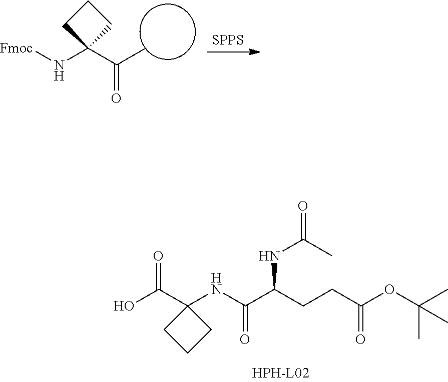

Experimental procedures:

Process step
1. 10.0 g of CTC Resin (CAS RN: 42074-68-0; 1.38 mmol/g,
10 mmol) was weighed and added to a 250 ml flask, and
dichloromethane (CAS RN: 75-09-2; 100 ml) was added;
7.74 g of N,N-diisopropylethylamine (CAS RN:
7087-68-5; 60 mmol) and 3.37 g of 1-((((9H-
fluoren-9-yl)methoxy)carbonyl)amino)cyclo-
butanecarboxylic acid (CAS RN: 885951-77-9;
10 mmol) were weighed and added to the reaction system.
2. The reaction mixture was stirred at a rotation speed
of 180 rpm at 20-25° C. for 4 hours.
3. Methanol (CAS RN: 67-56-1; 10 ml) was measured
and added to the reaction system, and the reaction mixture
was stirred at 20-25° C. at a rotation speed of 180 rpm for
another 1 hour.
4. The reaction mixture was transferred into a 150 ml
reactor, and the reaction mixture was removed by suction
filtration; the resin was washed with
N,N-dimethylformamide (CAS
RN: 68-12-2; 30 ml × 6).
5. 80 ml of N,N-dimethylformamide solution
containing 20% piperidine was added to
the reactor, and the reaction mixture was stirred
for 30 minutes at room temperature under
nitrogen atmosphere.
6. The reaction mixture was removed by suction
filtration, the resin was washed with
N,N-dimethylformamide (CAS RN: 68-12-2;
30 ml × 6), a small amount of resin (about 50
beads) was taken out, the resin was detected
with ninhydrin chromogenic solution, and both
the detection solution and the resin were dark blue.
7. 7.3 g of AC-GLU(OTBU)-OH (CAS RN: 84192-88-1;
30 mmol) and 11.4 g of HATU (CAS RN: 148893-10-1;
30 mmol) were weighed and added into N,N-
dimethylformamide (CAS RN: 68-12-2; 8 ml), 6.6 ml
of N-methylmorpholine (CAS RN: 109-02-4; 60 eq) was
slowly added at room temperature, and the reaction
mixture was stirred for 120 minutes at room
temperature under nitrogen atmosphere.
8. The reaction mixture was removed by suction
filtration, the resin was washed with
N,N-dimethylformamide (CAS RN: 68-12-2;
30 ml × 3), a small amount of resin (about 50
beads) was taken out and detected with
ninhydrin chromogenic solution, the detection
solution was pale yellow, and the resin was
colorless and transparent.
9. The reacted resin (11 g) was weighed and
added into a 250 ml eggplant-shaped flask,
then 30% HFIP/DCM (20 ml) was added, the
reaction mixture was stirred at 20-25° C. at a
rotation speed of 180 rpm for 2 hours; the reaction
mixture was filtered, the resin was washed with DCM
(10 ml × 3), and spin-dried to obtain the product (7.0 g).

LCMS: XT254984-036, Rt, =0.925 min, MS cal. 342.39, MS observed: [M+Na]⁺=365.2.

¹H NMR (400 MHz, DMSO-d₆) δ ppm 12.34 (s, 1H), 8.45 (s, 1H), 8.02 (d, J=8.0 Hz, 1H), 4.28-4.22 (m, 1H), 2.46-2.17 (m, 6H), 1.93-1.65 (m, 7H), 1.40 (s, 9H).

### Intermediate DSRB-C01: 2-(3,4-bis((4-(morpholine-4-carbonyl)phenyl)thio)-2,5-dioxo-2,5-dihydro-1H-pyrrol-1-yl)acetic acid

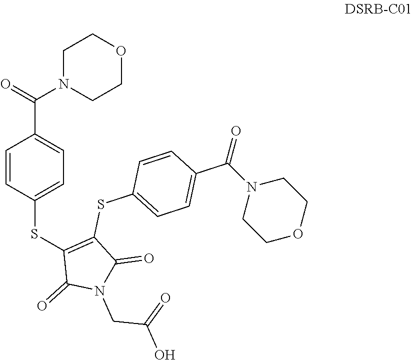 [SMILES: O=C(O)CN1C(=O)C(Sc2ccc(C(=O)N3CCOCC3)cc2)=C(Sc2ccc(C(=O)N3CCOCC3)cc2)C1=O]

The synthetic route for DSRB-C01 is shown below:

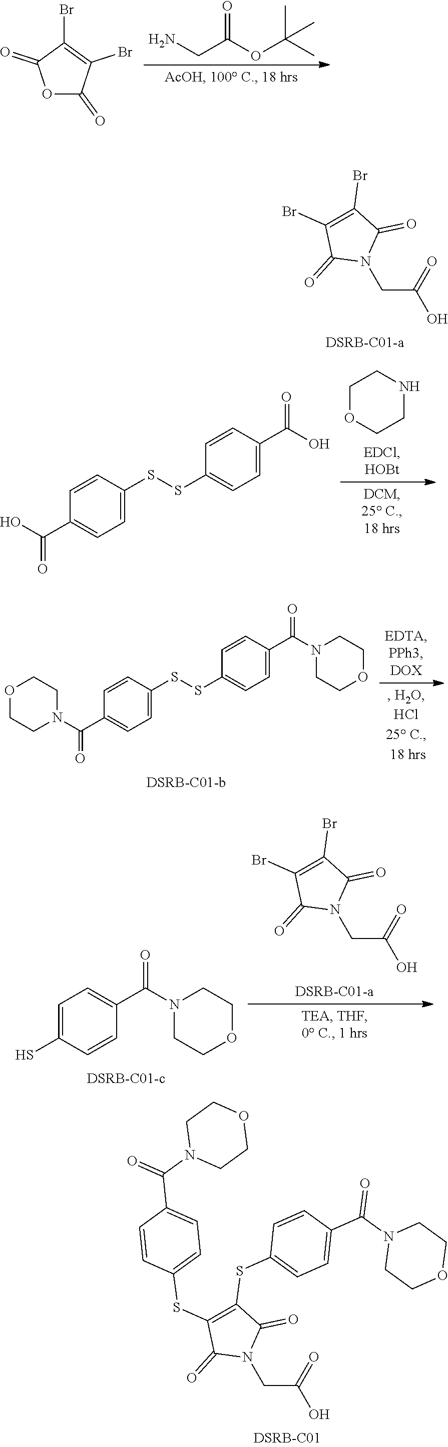

### Intermediate DSRB-C01-a: 2-(3,4-dibromo-2,5-dioxo-2,5-dihydro-1H-pyrrol-1-yl)acetic acid

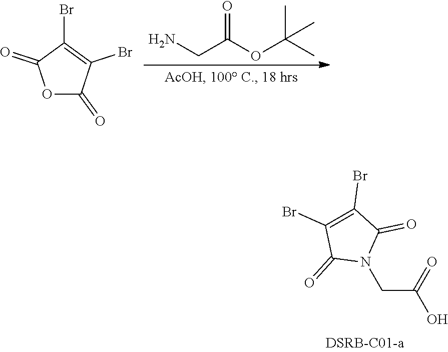

3,4-Dibromofuran-2,5-quinone (CAS RN: 1122-12-9, 1 g, 3.91 mmol) was dissolved in acetic acid (CAS RN: 64-19-7; 10 mL). Then tert-butyl glycinate (CAS RN: 6456-74-2, 513 mg, 3.91 mmol) was added to the reaction mixture at room temperature, and the reaction mixture was heated to 100° C. and stirred for 18 hours under nitrogen atmosphere. Then LC-MS was used to confirm the full consumption of starting materials in the reaction mixture. The solvent in the reaction mixture was reduced in vacuo to afford the reaction residue. The residue was then purified by a preparative reverse-phase liquid chromatograph (Column: Welch-Ultimate-C18-10 μm-21.2*150 mm, mobile phase A: 0.1% TFA in H₂O; mobile phase B: ACN) to recover the desired intermediate DSRB-C01-a (2-(3,4-dibromo-2,5-dioxo-2,5-dihydro-1H-pyrrol-1-yl)acetic acid; 160 mg, 0.39 mmol, 9.97% in yield) as a yellow oil.

¹H NMR (400 MHz, DMSO-d₆) δ 13.41 (s, 1H), 4.28 (s, 2H).

### Intermediate DSRB-C01-b: (disulfanediylbis(4,1-phenylene))bis (morpholinomethanone)

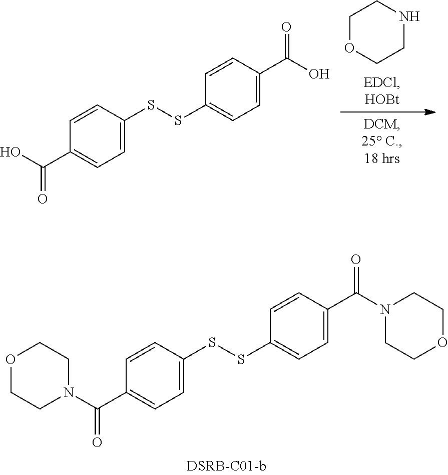

4,4′-Dithiobisbenzoic acid (CAS RN: 1155-51-7, 1 g, 3.26 mmol) was dissolved in DCM (CAS RN: 75-09-2; 15 mL) at room temperature. Then, HOBt (CAS RN: 2592-95-2, 1100 mg, 8.14 mmol) and EDCI (CAS RN: 25952-53-8; 1570 mg, 8.19 mmol) were added to the reaction mixture, and reaction mixture was left stirring for 10 minutes at room temperature. Then, morpholine (CAS RN: 110-91-8; 650 mg, 7.46 mmol) was added to the reaction mixture, and the reaction mixture was left stirring for another 18 hours at room temperature. LC-MS was used to confirm the full consumption of starting materials in the reaction mixture. The organic solvent in the reaction mixture was reduced in vacuo to afford the reaction residue. The residue was further purified by a preparative reverse-phase liquid chromatograph (Column: Welch-Ultimate-C18-10 μm-21.2*150 mm, mobile phase A: 0.1% NH₄HCO₃ in H₂O; mobile phase B: ACN) to recover the desired intermediate DSRB-C01-b ((disulfanediylbis(4,1-phenylene))bis(morpholinomethanone), 1300 mg, 1.72 mmol, 52.62% in yield) as a white solid.

LC-MS (m/z, C₂₂H₂₄N₂O₄S₂)=445.0[M+H]⁺.

¹H NMR (400 MHz, DMSO-d₆) δ 7.65-7.59 (m, 4H), 7.49-7.43 (m, 4H), 3.59-3.39 (m, 16H).

### Intermediate DSRB-C01-c: (4-mercaptophenyl)(morpholino)methanone

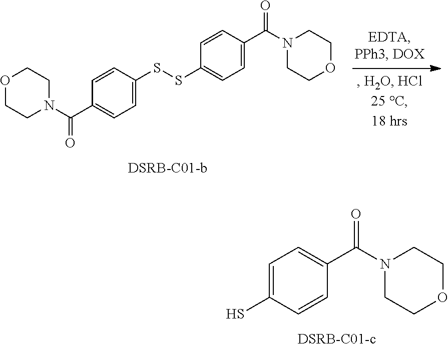 [SMILES: O=C(c1ccc(SSc2ccc(C(=O)N3CCOCC3)cc2)cc1)N1CCOCC1 ; O=C(c1ccc(S)cc1)N1CCOCC1]

Intermediate DSRB-C01-b ((disulfanediylbis(4,1-phenylene))bis(morpholinomethanone); 1900 mg, 4.27 mmol) was dissolved in a mixture of dioxane (CAS RN: 5703-4-8; 10 ml) and water (10 ml). Then EDTA (CAS RN: 60-00-4, 250 mg, 0.855 mmol), triphenylphosphine (CAS RN: 603-35-0; 2250 mg, 8.58 mmol) and hydrogen chloride (1 ml, 1 mol/L) were added to the reaction. Then the reaction mixture was left stirring at room temperature under nitrogen atmosphere for 15 hours. LC-MS was used to confirm the full consumption of starting materials in the reaction mixture. Ethyl acetate was used to extract the reaction mixture for 3 times (10 mL×3). The organic phase was separated and washed with 1 mol/L sodium hydroxide solution. The aqueous phase was combined and acidified by 1 mol/L hydrogen chloride solution until the pH value of the solution reached 3.0. Then ethyl acetate was used to extract the aqueous solution. The organic phase was combined and dried over anhydrous sodium sulphate. The organic solvent in the combined organic phase was reduced in vacuo to afford the desirable intermediate DSRB-C01-c ((4-mercaptophenyl)(morpholino)methanone, 1300 mg, 5.58 mmol, 69.24% in yield) as a colorless oil.

LC-MS (m/z, C₁H₁₃NO₂S)=224.0 [M+H]⁺.

¹H NMR (400 MHz, DMSO-d6) δ 7.38-7.36 (m, 2H), 7.31-7.29 (m, 2H), 5.75 (s, 1H), 3.60-3.35 (m, 8H).

### Intermediate DSRB-C01: 2-(3,4-bis((4-(morpholine-4-carbonyl)phenyl)thio)-2,5-dioxo-2,5-dihydro-1H-pyrrol-1-yl)acetic acid

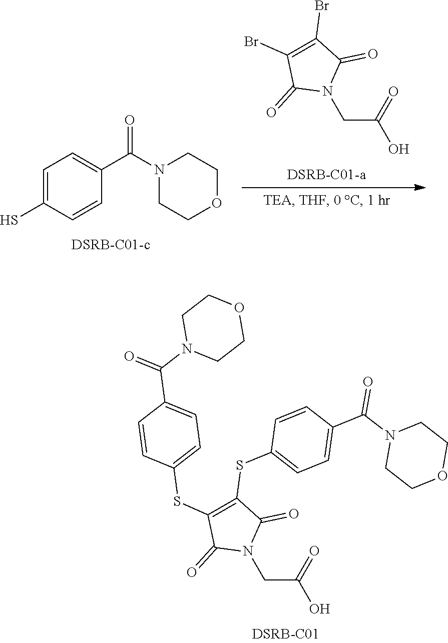 [SMILES: O=C(c1ccc(S)cc1)N1CCOCC1 ; O=C(O)CN1C(=O)C(Br)=C(Br)C1=O ; O=C(O)CN1C(=O)C(Sc2ccc(C(=O)N3CCOCC3)cc2)=C(Sc2ccc(C(=O)N3CCOCC3)cc2)C1=O]

Intermediate DSRB-C01-c ((4-mercaptophenyl)(morpholino)methanone; 72 mg, 0.32 mmol) and intermediate DSRB-C01-a (2-(3,4-dibromo-2,5-dioxo-2,5-dihydro-1H-pyrrol-1-yl)acetic acid; 50 mg, 0.16 mmol) were dissolved in THF (CAS RN: 109-99-9; 3 mL) at 0° C. Then triethylamine (CAS RN: 121-44-8; 109 mg, 1.08 mmol) was added to the reaction mixture, and the reaction mixture was left stirring at 0° C. under nitrogen atmosphere for 1 hour. LC-MS was used to confirm the full consumption of starting materials in the reaction mixture. Formic acid (CAS RN: 64-18-6) was used to adjust the pH of the reaction mixture to 5.0. Then, the organic solvent in the reaction mixture was reduced in vacuo to afford the reaction residue. The residue was further purified by a preparative reverse-phase liquid chromatograph (Column: Welch-Ultimate-C18-10 μm-21.2*150 mm, mobile phase A: 0.1% TFA in H₂O; mobile phase B: ACN) to recover the target intermediate DSRB-C01 (2-(3,4-bis((4-(morpholine-4-carbonyl)phenyl)thio)-2,5-dioxo-2,5-dihydro-1H-pyrrol-1-yl)acetic acid; 65 mg, 0.108 mmol, 67.91% in yield) as a yellow solid.

LC-MS (m/z, C₂₈H₂₇N₃O₈S₂)=598.2[M+H]⁺.

¹H NMR (400 MHz, DMSO-d6) δ 7.35-7.30 (m, 8H), 4.16 (s, 2H), 3.60 (s, 16H).

### Example 1: Payload P1

### Intermediate P1-b: tert-butyl (3-formyl-4-nitrophenyl) carbamate

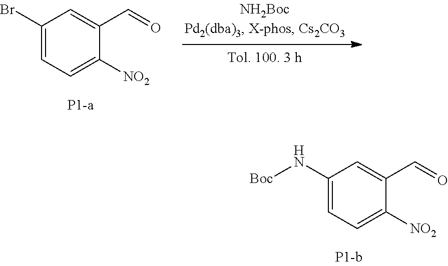 [SMILES: O=Cc1cc(Br)ccc1[N+](=O)[O-] ; CC(C)(C)OC(=O)Nc1ccc([N+](=O)[O-])c(C=O)c1]

A mixture of tert-butyl carbamate (CAS RN: 4248-19-5, 6.7 g, 57.19 mmol), tris(dibenzylideneacetone)dipalladium(0) (CAS RN: 51364-51-3, 2.4 g, 2.62 mmol), X-phos (CAS RN: 564483-18-7, 5.0 g, 10.49 mmol) and cesium carbonate (CAS RN: 534-17-8, 51 g, 156.53 mmol) was added to a toluene solution (CAS RN: 108-88-3, 180 ml) containing 5-bromo-2-nitrobenzaldehyde (intermediate P1-a, CAS RN: 20357-20-4, 12 g, 52.17 mmol). Then the reaction mixture was heated to 100° C. and stirred for 3 hours. LC-MS was used to confirm that the starting material was fully consumed. The reaction mixture was cooled to room temperature and added with distilled water (300 ml). The reaction mixture was further extracted with ethyl acetate twice (500 ml×2). The organic phase was combined, dried and concentrated in vacuo to yield a crude product. The crude product was purified via preparative silica chromatograph (petroleum ether:ethyl acetate=2:1) to yield the desired intermediate P1-b (tert-butyl (3-formyl-4-nitrophenyl)carbamate, 5.6 g, 21.03 mmol, 40.32% in yield) as a yellow solid.

LC-MS (m/z, C₁₂H₁₄N₂O₅)=267.0 [M+H]⁺.

¹H NMR (400 MHz, DMSO-d₆) δ 10.28 (m, 2H), 8.17 (m, 1H), 7.95 (m, 1H), 7.82 (m, 1H), 1.50 (s, 9H).

### Intermediate P1-c: tert-butyl (3-(1-hydroxy-3-(trimethylsilyl) prop-2-yn-1-yl)-4-nitrophenyl)carbamate

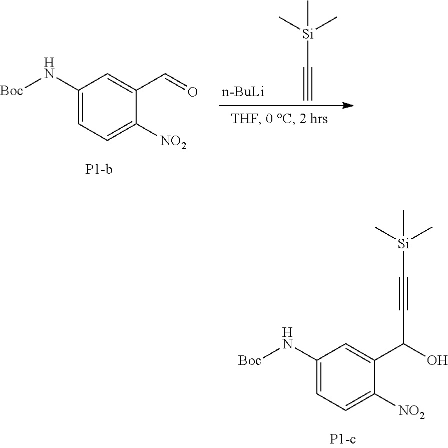

Ethynyltrimethylsilane (CAS RN: 1066-54-2; 498 mg, 5.07 mmol) was dissolved in tetrahydrofuran (CAS RN: 109-99-9, 10 mL) and cooled to 0° C. in anhydrous environment. Then a hexane solution of n-butyllithium (CAS RN: 109-72-8; 2.03 mL, 5.07 mmol, 2.5 mol/L in hexane) was added slowly to the tetrahydrofuran solution at 0° C. in anhydrous environment. The reaction mixture was stirred for 30 minutes at 0° C., followed by the addition of intermediate P1-b (tert-butyl (3-formyl-4-nitrophenyl) carbamate, 1 g, 3.76 mmol). The reaction mixture was then warmed to room temperature and stirred for additional 2 hours. LC-MS was used to confirm the consumption of remaining intermediate P1-b in the reaction. Saturated ammonium chloride solution (10 mL) was added to quench the reaction and ethyl acetate (100 mL×2) was used to extract the product from the reaction mixture. The organic phase was combined, dried over anhydrous sodium sulfate, filtered and evaporated in vacuo to provide the residue. The residue was then purified via silica chromatograph (petroleum ether:ethyl acetate=3:1) to yield the desired intermediate P1-c (tert-butyl (3-(1-hydroxy-3-(trimethylsilyl) prop-2-yn-1-yl)-4-nitrophenyl)carbamate, 800 mg, 2.19 mmol, 58.2% in yield) as a white solid.

LC-MS (m/z, C17H24N2O5Si)=347.2 [M−18]⁺.

¹H NMR (400 MHz, DMSO-d₆) δ 9.93 (s, 1H), 8.09 (m, 1H), 7.83 (m, 1H), 7.40-7.38 (m, 1H), 6.25 (m, 1H), 5.86 (m, 1H), 1.38 (s, 9H), 0.05 (s, 9H).

### Intermediate P1-d: tert-butyl (4-nitro-3-(3-(trimethylsilyl)propioloyl)phenyl) carbamate

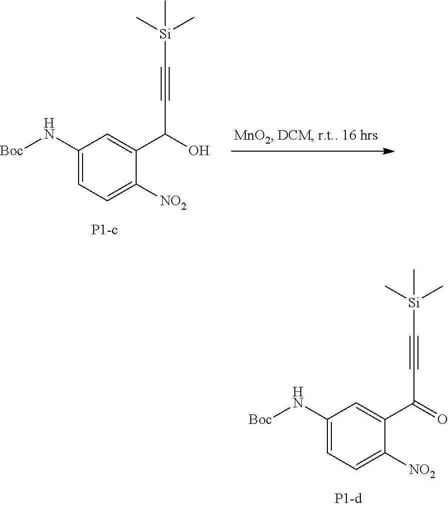 [SMILES: CC(C)(C)OC(=O)Nc1ccc([N+](=O)[O-])c(C(O)C#C[Si](C)(C)C)c1 ; "MnO2, DCM, r.t.. 16 hrs" ; CC(C)(C)OC(=O)Nc1ccc([N+](=O)[O-])c(C(=O)C#C[Si](C)(C)C)c1]

Intermediate P1-c (tert-butyl (3-(1-hydroxy-3-(trimethylsilyl)prop-2-yn-1-yl)-4-nitrophenyl)carbamate, 800 mg, 2.19 mmol) was dissolved in DCM (CAS RN: 75-09-2; 10 mL) at room temperature. Then Manganese(IV) oxide (CAS RN: 1313-13-9, 1.26 g, 14.50 mmol) was added to the reaction mixture and stirred for another 16 hours at room temperature. LC-MS was used to confirm the complete consumption of remaining intermediate P1-c in the reaction. The reaction mixture was filtered to remove the Manganese(IV) oxide and the organic filtrate was evaporated in vacuo to yield the desired intermediate P1-d as a white solid (tert-butyl (4-nitro-3-(3-(trimethylsilyl)propioloyl)phenyl)carbamate, 700 mg, 1.93 mmol, 88.12% in yield).

LC-MS (m/z, C17H22N2O5Si)=363.2 [M+H]⁺.

¹H NMR (400 MHz, DMSO-d₆) δ 10.29 (s, 1H), 8.17-8.00 (m, 2H), 7.76-7.74 (m, 1H), 1.50 (s, 9H), 0.25 (s, 9H).

### Intermediate P1-e: tert-butyl (4-amino-3-(3-(trimethylsilyl)propanoyl)phenyl)carbamate

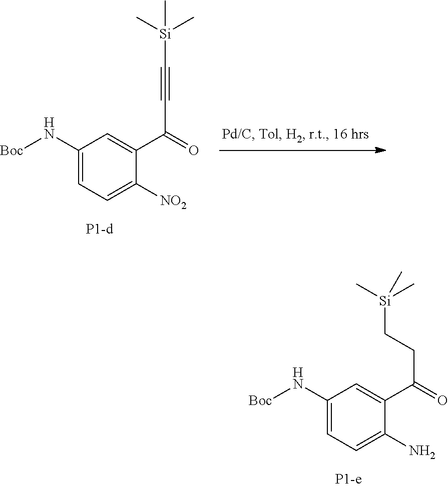 [SMILES: CC(C)(C)OC(=O)Nc1ccc([N+](=O)[O-])c(C(=O)C#C[Si](C)(C)C)c1 ; "Pd/C, Tol, H2, r.t., 16 hrs" ; CC(C)(C)OC(=O)Nc1ccc(N)c(C(=O)CC[Si](C)(C)C)c1]

Palladium on carbon support (CAS RN: 7440-05-3; 70 mg) was added to a toluene solution (CAS RN:108-88-3, 10 mL) of intermediate P1-d (tert-butyl (4-nitro-3-(3-(trimethylsilyl)propioloyl)phenyl)carbamate, 700 mg, 1.93 mmol) at room temperature. Then the reaction mixture was placed in hydrogen atmosphere and stirred for 16 hours at room temperature. LC-MS was used to confirm the complete consumption of remaining intermediate P1-d in the reaction. The reaction mixture was filtered and the organic filtrate was evaporated in vacuo to obtain the residue. The residue was purified via preparative silica chromatograph (petroleum ether:ethyl acetate=2:1) to yield the desired intermediate P1-e as a white solid (tert-butyl (4-amino-3-(3-(trimethylsilyl)propanoyl)phenyl) carbamate, 400 mg, 1.18 mmol, 61.13% in yield).

LC-MS (m/z, C17H28N2O3Si)=337.2[M+H]⁺.

¹H NMR (400 MHz, DMSO-d₆) δ 8.91 (s, 1H), 7.78 (s, 1H), 7.31 (s, 1H), 6.88 (s, 2H), 6.65 (m, 1H), 2.86-2.74 (m, 2H), 1.43 (s, 9H), 0.88-0.68 (m, 2H), 0.01 (s, 9H).

Payload P1: (S)-9-amino-4-ethyl-4-hydroxy-11-(2-(trimethylsilyl)ethyl)-1,12-dihydro-14H-pyrano[3′,4′:6,7]indolizino[1,2-b]quinoline-3,14(4H)-dione

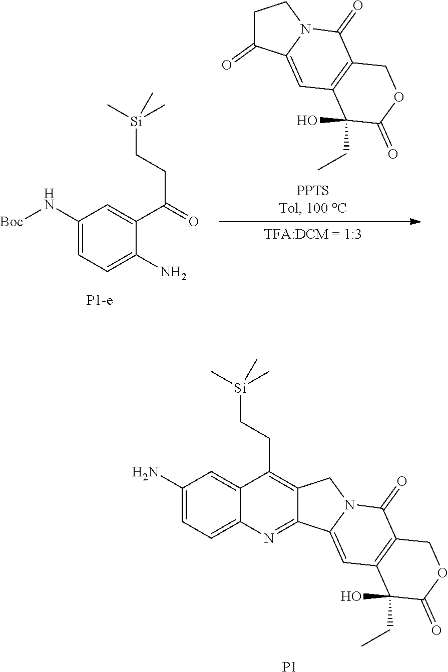

Intermediate P1-e (tert-butyl (4-amino-3-(3-(trimethylsilyl)propanoyl)phenyl)carbamate, 300 mg, 0.89 mmol) was dissolved in 10 ml toluene (CAS RN: 108-88-3) at room temperature. The toluene solution was then added with pyridinium p-toluenesulfonate (CAS RN: 24057-28-1, 14 mg, 0.05 mmol) and (S)-4-ethyl-4-hydroxy-7,8-dihydro-1H-pyrano[3,4-f]indolizine-3,6,10(4H)-trione (CAS RN: 110351-94-5; 211 mg, 0.8 mmol). The reaction mixture was then heated to 100° C. and stirred at this temperature for 16 hours. LC-MS was used to confirm the complete consumption of remaining intermediate P1-e in the reaction. The reaction mixture was then cooled to room temperature and the organic solvent was evaporated in vacuo to afford the residue. The residue was added with a mixture of DCM (CAS RN: 75-09-2, 6 ml) and trifluoroacetic acid (CAS RN: 76-05-1, 3 ml) and stirred at room temperature for an additional 2 hours. The organic solvent and acid were reduced in vacuo again to obtain the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to obtain the final payload P1 as a white solid ((S)-9-amino-4-ethyl-4-hydroxy-11-(2-(trimethylsilyl)ethyl)-1,12-dihydro-14H-pyrano[3′,4′:6,7]indolizino[1,2-b]quinoline-3,14(4H)-dione, 130 mg, 0.28 mmol, 35.0% in yield).

LC-MS (m/z, C25H29N3O4Si)=464.2[M+H]⁺.

¹H NMR (400 MHz, DMSO-d₆) δ 7.69 (m, 1H), 7.11-7.08 (m, 1H), 7.02 (s, 1H), 6.84 (m, 1H), 6.27 (s, 1H), 5.24 (s, 2H), 5.05 (s, 2H), 2.83-2.73 (m, 2H), 1.75-1.64 (m, 2H), 0.76-0.70 (m, 5H), 0.01 (s, 9H).

### Example 2: Payload P2

### Intermediate P2-b: tert-butyl (2-fluoro-5-formyl-4-nitrophenyl) carbamate

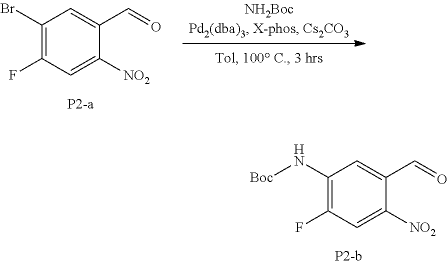 [SMILES: O=Cc1cc(Br)c(F)cc1[N+](=O)[O-] ; CC(C)(C)OC(=O)Nc1cc(C=O)c([N+](=O)[O-])cc1F]

A mixture of tert-butyl carbamate (CAS RN: 4248-19-5, 12.47 g, 106.45 mmol), tris(dibenzylideneacetone)dipalladium(0) (CAS RN: 51364-51-3, 4.43 g, 4.84 mmol), X-phos (CAS RN: 564483-18-7, 9.22 g, 19.35 mmol) and cesium carbonate (CAS RN: 534-17-8, 94.59 g, 290.31 mmol) was added to a toluene solution (CAS RN: 108-88-3, 360 ml) containing 5-bromo-4-fluoro-2-nitrobenzaldehyde (intermediate P2-a, CAS RN: 213382-45-7; 24 g, 96.77 mmol) at room temperature. Then the reaction mixture was heated to 100° C. and stirred at this temperature for 3 hours. The reaction mixture was cooled to room temperature and added with distilled water (500 ml). The reaction mixture was further extracted with ethyl acetate twice (1000 ml×2). The organic phase was combined, dried and concentrated in vacuo to yield a crude product. The crude product was purified via preparative silica chromatograph (petroleum ether:ethyl acetate=1:1) to yield the desired intermediate P2-b (tert-butyl (2-fluoro-5-formyl-4-nitrophenyl) carbamate, 22 g, 77.40 mmol, 79.98% in yield) as a yellow solid.

LC-MS (m/z, C12H13FN2O5)=285.0 [M+H]⁺.

¹H NMR (400 MHz, DMSO-d₆) δ 10.24 (s, 1H), 9.95 (m, 1H), 8.42 (m, 1H), 8.18 (m, 1H), 1.50 (s, 9H).

### Intermediate P2-c: tert-butyl (2-fluoro-5-(1-hydroxy-3-(trimethylsilyl)prop-2-yn-1-yl)-4-nitrophenyl)carbamate

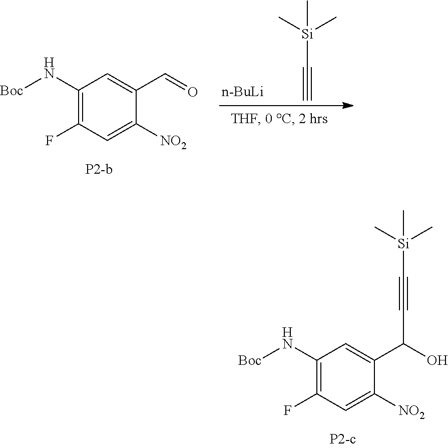

Ethynyltrimethylsilane (CAS RN: 1066-54-2; 196.97 mg, 2.01 mmol) was dissolved in tetrahydrofuran (CAS RN: 109-99-9, 80 mL) and cooled to 0° C. in anhydrous environment. Then a hexane solution of n-butyllithium (CAS RN: 109-72-8; 0.8 mL, 2.01 mmol, 2.5 mol/L in hexane) was added slowly to the tetrahydrofuran solution at 0° C. in anhydrous environment. The reaction mixture was stirred for 30 minutes at 0° C., followed by the addition of intermediate P2-b (tert-butyl (2-fluoro-5-formyl-4-nitrophenyl) carbamate, 400 mg, 1.40 mmol). The reaction mixture was then warmed to room temperature and stirred for additional 2 hours. LC-MS was used to confirm the consumption of remaining intermediate P1-b in the reaction. Saturated ammonium chloride solution (10 mL) was added to quench the reaction and ethyl acetate (50 mL×2) was used to extract the product from the reaction mixture. The organic phase was combined, dried over anhydrous sodium sulfate, filtered and evaporated in vacuo to provide the residue. The residue was then purified via silica chromatograph (petroleum ether:ethyl acetate=3:1) to yield the desired intermediate P2-c (tert-butyl (2-fluoro-5-(1-hydroxy-3-(trimethylsilyl)prop-2-yn-1-yl)-4-nitrophenyl)carbamate, 450 mg, 1.18 mmol, 84.28% in yield) as a white solid.

LC-MS (m/z, C17H23FN2O5Si)=365.2[M−18]⁺.

¹H NMR (400 MHz, DMSO-d₆) δ 9.56 (m, 1H), 8.42 (m, 1H), 7.83 (m, 1H), 6.32 (m, 1H), 5.81 (m, 1H), 1.36 (s, 9H), 0.02 (s, 9H).

### Intermediate P2-d: tert-butyl (2-fluoro-4-nitro-5-(3-(trimethylsilyl)propioloyl)phenyl)carbamate

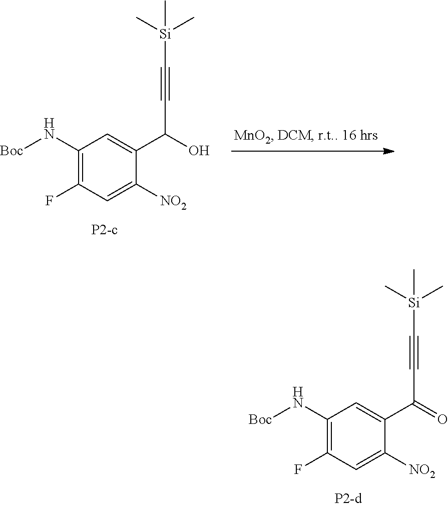 [SMILES: CC(C)(C)OC(=O)Nc1cc(C(O)C#C[Si](C)(C)C)c([N+](=O)[O-])cc1F ; "MnO2, DCM, r.t.. 16 hrs" ; CC(C)(C)OC(=O)Nc1cc(C(=O)C#C[Si](C)(C)C)c([N+](=O)[O-])cc1F]

Intermediate P2-c (tert-butyl (2-fluoro-5-(1-hydroxy-3-(trimethylsilyl)prop-2-yn-1-yl)-4-nitrophenyl)carbamate, 450 mg, 1.18 mmol) was dissolved in DCM (CAS RN: 75-09-2; 10 mL) at room temperature. Then Manganese(IV) oxide (CAS RN: 1313-13-9, 680.25 g, 7.82 mmol) was added to the reaction mixture and stirred for another 16 hours at room temperature. LC-MS was used to confirm the complete consumption of remaining intermediate P2-c in the reaction. The reaction mixture was filtered to remove the Manganese(IV) oxide and the organic filtrate was evaporated in vacuo to yield the desired intermediate P2-d as a white solid (tert-butyl (2-fluoro-4-nitro-5-(3-(trimethylsilyl)propioloyl)phenyl)carbamate, 400 mg, 1.05 mmol, 88.98% in yield).

LC-MS (m/z, C17H21FN2O5Si)=381.0 [M+H]⁺.

### Intermediate P2-e: tert-butyl (4-amino-2-fluoro-5-(3-(trimethylsilyl)propanoyl)phenyl)carbamate

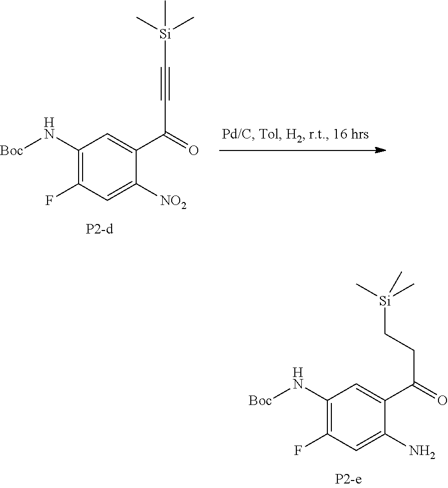 [SMILES: CC(C)(C)OC(=O)Nc1cc(C(=O)C#C[Si](C)(C)C)c([N+](=O)[O-])cc1F ; "Pd/C, Tol, H2, r.t., 16 hrs" ; CC(C)(C)OC(=O)Nc1cc(C(=O)CC[Si](C)(C)C)c(N)cc1F]

Palladium on carbon support (CAS RN: 7440-05-3; 30 mg) was added to a toluene solution (CAS RN: 108-88-3, 10 mL) of intermediate P2-d (tert-butyl (2-fluoro-4-nitro-5-(3-(trimethylsilyl)propioloyl)phenyl)carbamate, 400 mg, 1.05 mmol) at room temperature. Then the reaction mixture was placed in hydrogen atmosphere and stirred for 16 hours at room temperature. LC-MS was used to confirm the complete consumption of remaining intermediate P1-d in the reaction. The reaction mixture was filtered and the organic filtrate was evaporated in vacuo to obtain the residue. The residue was purified via preparative silica chromatograph (petroleum ether:ethyl acetate=2:1) to yield the desired intermediate P2-e as a white solid (tert-butyl (4-amino-2-fluoro-5-(3-(trimethylsilyl)propanoyl)phenyl)carbamate, 300 mg, 0.85 mmol, 80.71% in yield).

LC-MS (m/z, C17H27FN2O3Si)=355.2[M+H]⁺.

¹H NMR (400 MHz, DMSO-d₆) δ 8.48 (s, 1H), 7.72 (m, 1H), 7.26 (s, 2H), 6.51 (m, 1H), 2.96-2.72 (m, 2H), 1.41 (s, 9H), 0.92-0.69 (m, 2H), 0.01 (s, 9H).

### Payload P2: (S)-9-amino-4-ethyl-8-fluoro-4-hydroxy-11-(2-(trimethylsilyl)ethyl)-1,12-dihydro-14H-pyrano[3′,4′:6,7]indolizino[1,2-b]quinoline-3,14(4H)-dione

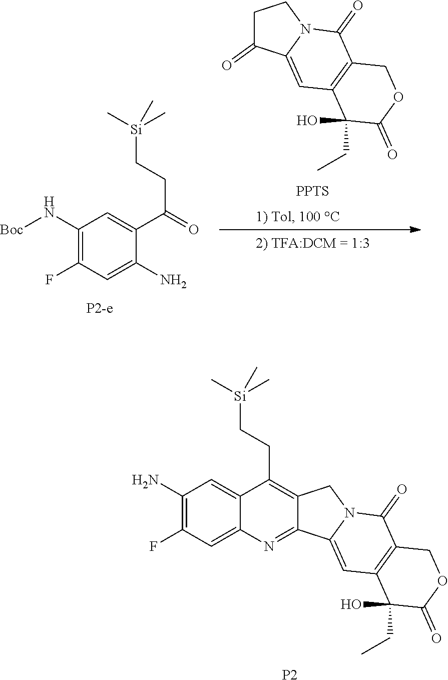

Intermediate P2-e (tert-butyl (4-amino-2-fluoro-5-(3-(trimethylsilyl)propanoyl)phenyl)carbamate, 277 mg, 0.74 mmol) was dissolved in 10 ml toluene (CAS RN: 108-88-3) at room temperature. The toluene solution was then added with pyridinium p-toluenesulfonate (CAS RN: 24057-28-1, 12.78 mg, 0.07 mmol) and (S)-4-ethyl-4-hydroxy-7,8-dihydro-1H-pyrano[3,4-f]indolizine-3,6,10(4H)-trione (CAS RN: 110351-94-5; 195.41 mg, 0.74 mmol). The reaction mixture was then heated to 100° C. and stirred at this temperature for 16 hours. LC-MS was used to confirm the complete consumption of remaining intermediate P2-e in the reaction. The reaction mixture was then cooled to room temperature and the organic solvent was evaporated in vacuo to afford the residue. The residue was added with a mixture of DCM (CAS RN: 75-09-2, 6 ml) and trifluoroacetic acid (CAS RN: 76-05-1, 3 ml) and stirred at room temperature for an additional 2 hours. The organic solvent and acid were reduced in vacuo again to obtain the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to obtain the final payload P2 as a white solid ((S)-9-amino-4-ethyl-8-fluoro-4-hydroxy-11-(2-(trimethylsilyl)ethyl)-1,12-dihydro-14H-pyrano[3′,4′:6,7]indolizino[1,2-b]quinoline-3,14(4H)-dione, 124 mg, 0.26 mmol, 35.13% in yield).

LC-MS (m/z, C25H28FN3O4Si)=482.2[M+H]⁺.

¹H NMR (400 MHz, DMSO-d₆) δ 7.58 (m, 1H), 7.11-7.01 (m, 2H), 6.33 (s, 1H), 5.25 (s, 2H), 5.06 (s, 2H), 2.84-2.76 (m, 2H), 1.73-1.66 (m, 2H), 0.7600.68 (m, 5H), 0.01 (s, 9H).

### Example 3: Linker-Payload L7P1

### Intermediate L7-a: 5-(tert-butyl) 1-(2,5-dioxopyrrolidin-1-yl) (((9H-fluoren-9-yl)methoxy)carbonyl)-L-glutamate

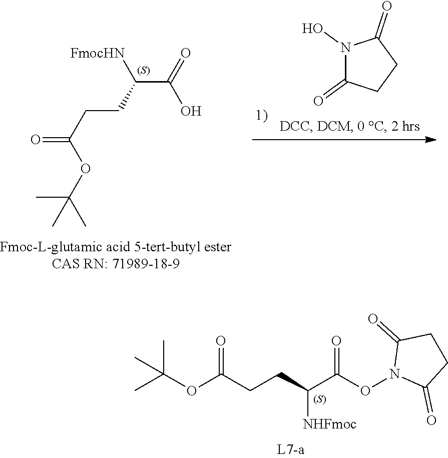

Fmoc-L-glutamic acid 5-tert-butyl ester (CAS RN: 71989-18-9; 1 g, 2.35 mmol) was dissolved in 15 ml DCM (CAS RN: 75-09-2) and cooled to 0° C. Then, HOSu (CAS RN: 6066-82-6; 0.30 g, 2.59 mmol) and DCC (CAS RN: 538-75-0; 0.58 g, 2.81 mmol) were added to the mixture at 0° C. The reaction mixture was left stirring at 0° C. for another 30 minutes. Samples of reaction mixture were taken and analyzed by LC-MS to confirm the full conversion of Fmoc-L-glutamic acid 5-tert-butyl ester to its O-succinate ester. The reaction mixture was then filtered and the filtrate was collected. The organic solvent in the filtrate was evaporated in vacuo to afford the crude intermediate L7-a as a white solid (5-(tert-butyl) 1-(2,5-dioxopyrrolidin-1-yl) (((9H-fluoren-9-yl)methoxy)carbonyl)-L-glutamate; 1.1 g, 2.11 mmol, 89.79% in yield).

LC-MS (m/z, C₂₈H₃₀N₂O₈)=540.2 [M+H₂O]⁺.

¹H NMR (400 MHz, DMSO-d₆) δ 8.12 (m, 1H), 7.90 (m, 2H), 7.71 (m, 2H), 7.44-7.40 (m, 2H), 7.33 (m, 2H), 4.56-4.50 (m, 1H), 4.35-4.32 (m, 2H), 4.26-4.23 (m, 1H), 2.81 (s, 4H), 2.44-2.39 (m, 2H), 2.14-2.06 (m, 1H), 1.99-1.90 (m, 1H), 1.40 (s, 9H).

### Intermediate L7-b: (S)-3-(2-((((9H-fluoren-9-yl)methoxy)carbonyl)amino)-5-(tert-butoxy)-5-oxopentanamido)oxetane-3-carboxylic acid

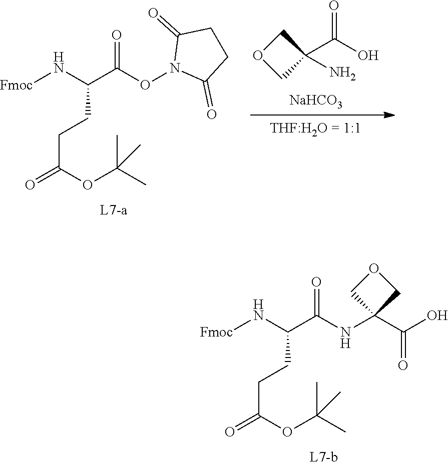

Intermediate L7-a (5-(tert-butyl) 1-(2,5-dioxopyrrolidin-1-yl) (((9H-fluoren-9-yl)methoxy)carbonyl)-L-glutamate; 1.1 g, 2.11 mmol) was dissolved in a mixture of water and tetrahydrofuran (volume H₂O:volume THF=1:1, 16 mL) and cooled to 0° C. Then, 3-amino-3-oxetanecarboxylic acid (CAS RN: 138650-24-5; 370 mg, 3.16 mmol) and sodium bicarbonate (CAS RN: 144-55-8; 350 mg, 4.17 mmol) were added to the mixture and the reaction mixture was left stirring at 0° C. for a further 30 minutes. Samples of reaction mixture were taken and analyzed by LC-MS to confirm the full consumption of intermediate L7-a in the mixture. Then the organic solvent in the reaction mixture was evaporated in vacuo and the resulting residue was purified directly using a preparative reverse-phase liquid chromatograph (Column: Welch-Ultimate-C18-10 μm-21.2*150 mm, mobile phase A: 0.1% TFA in H₂O; mobile phase B: ACN) to yield a white solid as the target intermediate L7-b ((S)-3-(2-((((9H-fluoren-9-yl)methoxy)carbonyl)amino)-5-(tert-butoxy)-5-oxopentanamido)oxetane-3-carboxylic acid; 490 mg, 0.93 mmol, 44.31% in yield).

LC-MS (m/z, C₂₈H₃₂N₂O₈)=525.2[M+H]⁺.

¹H NMR (400 MHz, DMSO-d₆) δ 8.80 (s, 1H), 7.89 (m, 2H), 7.74 (m, 2H), 7.60 (m, 1H), 7.44-7.40 (m, 2H), 7.35-7.31 (m, 2H), 4.81 (m, 1H), 4.73 (m, 1H), 4.63-4.61 (m, 1H), 4.59-4.51 (m, 1H), 4.30-4.20 (m, 3H), 4.05-4.01 (m, 1H), 2.33-2.25 (m, 2H), 1.95-1.90 (m, 1H), 1.80-1.72 (m, 1H), 1.39 (s, 9H).

### Intermediate L7-c: 3-((2-(2,5-dioxo-2,5-dihydro-1H-pyrrol-1-yl)ethyl)(methyl)amino)propanoic acid

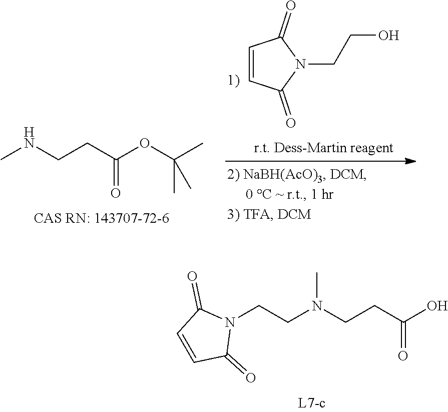 [SMILES: O=C1C=CC(=O)N1CCO ; CNCCC(=O)OC(C)(C)C ; CN(CCC(=O)O)CCN1C(=O)C=CC1=O]

1-(2-Hydroxyethyl)maleimide (CAS RN: 1585-90-6; 2.1 g, 14.9 mmol) was dissolved in DCM (CAS RN: 75-09-2; 10 ml) and cooled to 0° C. Then, Dess-Martin periodinate (CAS RN: 87413-09-0; 6.31 mg, 14.9 mmol) was added to the reaction mixture and the reaction mixture was warmed to room temperature and left stirring for 30 minutes. The reaction mixture was filtered. The resulting filtrate was collected and added with tert-butyl 3-(methylamino)propanoate (CAS RN: 143707-72-6; 2.0 g, 12.6 mmol) dissolved in 5 mL DCM (CAS RN: 75-09-2) and sodium triacetoxyborohydride (CAS RN: 56553-60-7; 3.8 g, 17.9 mmol). The reaction mixture was left stirring under room temperature for another 30 minutes before being cooled to 0° C. again. Trifluoroacetic acid (CAS RN: 76-05-1) was added to the mixture under 0° C. and the reaction mixture was stirred for a further 2 hours at 0° C. Ethyl acetate (10 ml×3) was added to the reaction mixture to extract the desired product. The organic phase was combined, dried over anhydrous sodium sulfate and evaporated in vacuo to provide the oily residue. The residue was then purified via a preparative reverse-phase liquid chromatograph (Column: Welch-Ultimate-C18-10 μm-21.2*150 mm, mobile phase A: 0.1% TFA in H₂O; mobile phase B: ACN) to recover an oil as the desired intermediate L7-c (3-((2-(2,5-dioxo-2,5-dihydro-1H-pyrrol-1-yl)ethyl)(methyl)amino)propanoic acid, 1.25 g, 5.54 mmol, 44% in yield).

LC-MS (m/z, C₁₀H₁₄N₂O₄)=227.2[M+H]⁺.

¹H NMR (400 MHz, CDCl₃) δ 6.69 (s, 2H), 3.89 (m, 2H), 3.45 (m, 2H), 3.35 (m, 2H) 2.91 (s, 3H), 2.86 (m, 2H).

### Intermediate L7P1-a: tert-butyl (S)-(2-((4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

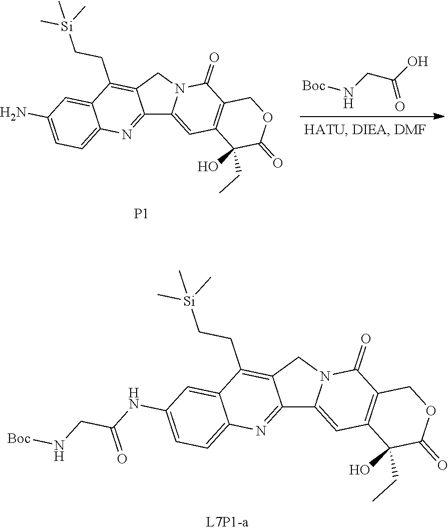 [SMILES: CC[C@@]1(O)C(=O)OCc2c1cc1n(c2=O)Cc2c-1nc1ccc(N)cc1c2CC[Si](C)(C)C ; CC(C)(C)OC(=O)NCC(=O)O ; "P1" ; CC[C@@]1(O)C(=O)OCc2c1cc1n(c2=O)Cc2c-1nc1ccc(NC(=O)CNC(=O)OC(C)(C)C)cc1c2CC[Si](C)(C)C]

Boc-glycine (CAS RN: 4530-20-5; 4.72 g, 26.95 mmol) was dissolved in DMF (CAS RN: 68-12-2; 50 ml) and cooled to 0° C. The reaction mixture was then added with HATU (CAS RN: 148893-10-1; 10.25 g, 26.96 mmol) and DIPEA (CAS RN: 7087-68-5; 3.48 g, 26.92 mmol) and left stirring at 0° C. for 1 hour. Payload P1 ((S)-9-amino-4-ethyl-4-hydroxy-11-(2-(trimethylsilyl)ethyl)-1,12-dihydro-14H-pyrano[3′,4′:6,7]indolizino[1,2-b]quinoline-3,14(4H)-dione; 2.5 g, 5.39 mmol) was added to the reaction mixture and the reaction mixture was warmed to room temperature to react overnight. LC-MS was employed to verify the complete consumption of Payload P1 in the reaction mixture. The organic solvent in the mixture was reduced in vacuo again to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-a as a yellow solid (tert-butyl (S)-(2-((4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate, 3000 mg, 4.83 mmol, 89.61% in yield).

LC-MS (m/z, C32H40N4O7Si)=621.2 [M+H]⁺.

¹H NMR (400 MHz, DMSO-d₆) δ 10.41 (s, 1H), 8.72 (s, 1H), 8.13 (m, 1H), 7.87 (m, 1H), 7.30 (s, 1H), 7.17 (m, 1H), 6.50 (s, 1H), 5.45 (s, 2H), 5.31 (s, 2H), 3.84 (m, 2H), 3.10-3.01 (m, 2H), 1.93-1.85 (m, 2H), 1.43 (s, 9H), 1.00-0.90 (m, 5H), 0.19 (s, 9H).

### Intermediate L7P1-b: (S)-2-amino-N-(4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide

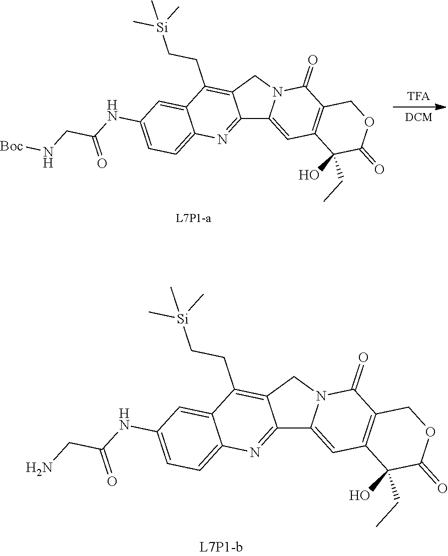 [SMILES: CC[C@@]1(O)C(=O)OCc2c1cc1n(c2=O)Cc2c-1nc1ccc(NC(=O)CNC(=O)OC(C)(C)C)cc1c2CC[Si](C)(C)C ; CC[C@@]1(O)C(=O)OCc2c1cc1n(c2=O)Cc2c-1nc1ccc(NC(=O)CN)cc1c2CC[Si](C)(C)C]

Intermediate L7P1-a (tert-butyl (S)-(2-((4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate, 2.5 g, 4.03 mmol) was dissolved in DCM (CAS RN: 75-09-2, 50 mL) at room temperature. Then, trifluoroacetic acid (CAS RN: 76-05-1; 10 mL, 130.68 mmol) was added to the reaction mixture and stirred for 90 minutes at room temperature. LC-MS was employed to verify the complete consumption of intermediate L7P1-a in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-b as a yellow solid ((S)-2-amino-N-(4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide, 2023 mg, 3.26 mmol, 80.89% in yield).

LC-MS (m/z, C27H32N4O5Si)=521.2[M+H]⁺.

¹H NMR (400 MHz, DMSO-d₆) δ 8.70 (m, 1H), 8.19 (m, 4H), 7.88 (m, 1H), 7.32 (s, 1H), 6.54 (s, 1H), 5.45 (s, 2H), 5.34 (s, 2H), 3.92 (s, 2H), 3.12-3.03 (m, 2H), 1.98-1.85 (m, 2H), 1.01-0.90 (m, 5H), 0.20 (s, 9H).

### Intermediate L7P1-c: (2R,3R,4S,5S,6R)-2-((S)-2-((((9H-fluoren-9-yl)methoxy)carbonyl)amino)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate

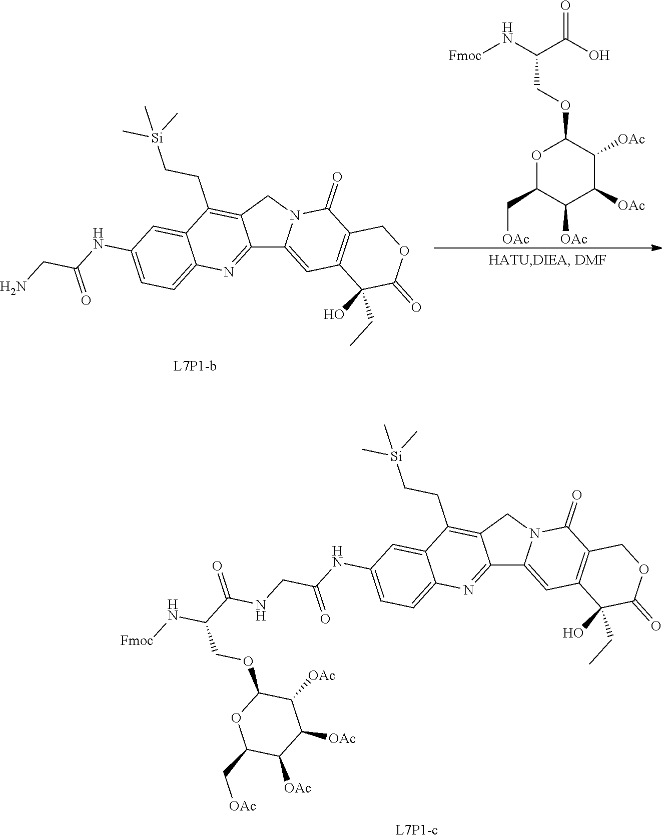

N-(((9H-Fluoren-9-yl)methoxy)carbonyl)-O-((2R,3R,4S,5S,6R)-3,4,5-triacetoxy-6-(acetoxymethyl)tetrahydro-2H-pyran-2-yl)-L-serine (CAS RN: 96383-44-7; 947.29 mg, 1.44 mmol) was dissolved in 15 ml DMF (CAS RN: 68-12-2) under 0° C. The reaction mixture was then added with HATU (CAS RN: 148893-10-1; 821.59 mg, 2.16 mmol) and DIPEA (CAS RN: 7087-68-5; 372.36 mg, 2.88 mmol) and left stirring for 30 minutes under 0° C. Intermediate L7P1-b ((S)-2-amino-N-(4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide, 750 mg, 1.44 mmol) was added to the reaction mixture and the reaction mixture was left stirring for additional 30 minutes under 0° C. LC-MS was employed to verify the complete conversion of intermediate L7P1-b in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-c as a yellow solid ((2R,3R,4S,5S,6R)-2-((S)-2-((((9H-fluoren-9-yl)methoxy)carbonyl)amino)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate; 1200 mg, 1.03 mmol, 71.80% in yield).

LC-MS (m/z, C59H65N5O18Si)=1160.4 [M+H]⁺.

¹H NMR (400 MHz, DMSO-d₆) δ 10.33 (s, 1H), 8.68 (d, J=2.2 Hz, 1H), 8.50 (t, J=5.7 Hz, 1H), 8.12 (d, J=9.1 Hz, 1H), 7.1-7.88 (m, 4H), 7.74 (t, J=6.9 Hz, 2H), 7.65 (d, J=7.9 Hz, 1H), 7.45-7.39 (m, 3H), 7.35 (t, J=6.8 Hz, 3H), 7.31 (s, 1H), 5.45 (s, 2H), 5.30 (s, 2H), 5.30-5.29 (m, 1H), 4.92-4.82 (m, 1H), 4.81 (d, J=8.0 Hz, 1H), 4.26-4.23 (m, 4H), 4.08 (d, J=7.3 Hz, 4H), 3.87 (t, J=4.9 Hz, 2H), 3.03 (s, 2H), 2.12 (s, 3H), 2.02 (s, 3H), 1.98 (s, 3H), 1.93 (s, 3H), 1.91-1.86 (m, 2H), 0.88-0.90 (m, 5H), 0.16 (s, 9H).

### Intermediate L7P1-d: (2R,3S,4S,5R,6R)-2-(acetoxymethyl)-6-((S)-2-amino-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)tetrahydro-2H-pyran-3,4,5-triyl triacetate

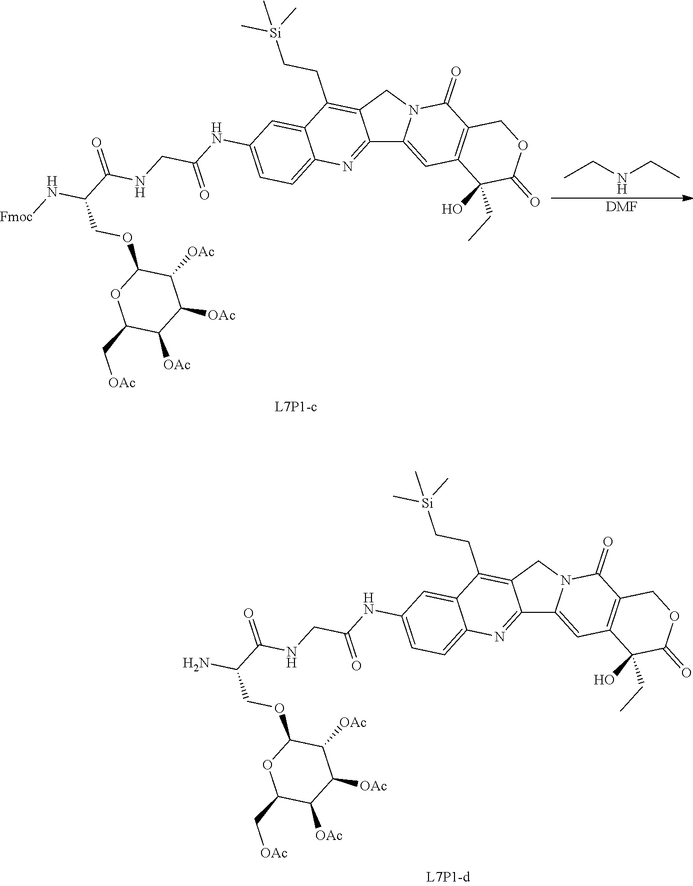

Intermediate L7P1-c ((2R,3R,4S,5S,6R)-2-((S)-2-((((9H-fluoren-9-yl)methoxy)carbonyl)amino)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate, 0.41 g, 0.35 mmol) was dissolved in 10 mL DMF (CAS RN: 68-12-2) under 0° C. Then diethylamine (CAS RN: 109-89-7; 0.36 mL, 3.53 mmol) was added to the reaction mixture and stirred for 2 hours under 0° C. LC-MS was employed to verify the complete conversion of intermediate L7P1-c in the reaction mixture. The organic solvent and excess amount of diethylamine in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-d as a yellow solid ((2R,3S,4S,5R,6R)-2-(acetoxymethyl)-6-((S)-2-amino-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)tetrahydro-2H-pyran-3,4,5-triyl triacetate; 310 mg, 0.33 mmol, 94.42% in yield).

LC-MS (m/z, C44H55N5O16Si)=960.2[M+23]⁺.

¹H NMR (400 MHz, DMSO-d₆) ¹H NMR (400 MHz, DMSO) δ 10.62 (s, 1H), 8.94 (t, J=5.8 Hz, 1H), 8.66 (d, J=2.3 Hz, 1H), 8.28 (d, J=4.9 Hz, 2H), 8.16 (d, J=9.1 Hz, 1H), 7.98-7.90 (m, 1H), 7.31 (s, 1H), 5.45 (s, 2H), 5.33 (s, 2H), 5.30 (d, J=3.6 Hz, 1H), 5.21 (dd, J=10.3, 3.6 Hz, 1H), 5.01-4.99 (m, 1H), 4.84-4.83 (m, 1H), 4.28 (s, 1H), 4.20-4.08 (m, 6H), 4.02-4.01 (m, 2H), 3.11-3.02 (m, 2H), 2.13 (s, 3H), 2.07 (s, 3H), 2.02 (s, 3H), 1.93 (d, J=4.3 Hz, 3H), 1.92-1.85 (m, 2H), 0.94 (d, J=7.4 Hz, 2H), 0.90 (t, J=7.3 Hz, 3H), 0.19 (s, 9H).

### Intermediate L7P1-e: (2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate

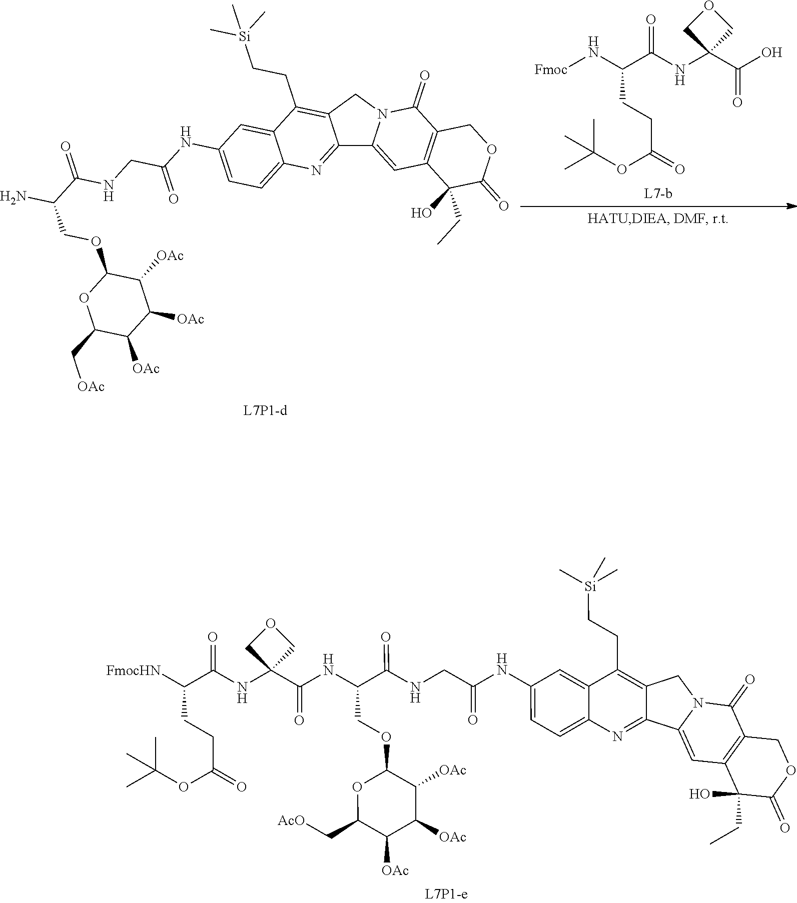

Intermediate L7-b ((S)-3-(2-((((9H-fluoren-9-yl)methoxy)carbonyl)amino)-5-(tert-butoxy)-5-oxopentanamido)oxetane-3-carboxylic acid; 154.35 mg, 0.29 mmol) was dissolved in 5 ml DMF (CAS RN: 68-12-2) under 0° C. The reaction mixture was then added with HATU (CAS RN: 148893-10-1; 139.85 mg, 0.37 mmol) and DIPEA (CAS RN: 7087-68-5; 63.38 mg, 0.49 mmol) and left stirring for 30 minutes under 0° C. Intermediate L7P1-d ((2R,3S,4S,5R,6R)-2-(acetoxymethyl)-6-((S)-2-amino-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)tetrahydro-2H-pyran-3,4,5-triyl triacetate, 230 mg, 0.25 mmol) was added to the reaction mixture and the reaction mixture was left stirring for additional 30 minutes under 0° C. LC-MS was employed to verify the complete conversion of intermediate L7P1-d in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-e as a yellow solid ((2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate, 180 mg, 0.12 mmol, 48.0% in yield).

LC-MS (m/z, C72H85N7O23Si)=1445.4 [M+H]⁺& 723.0 [M+2H]⁺.

### Intermediate L7P1-f: tert-butyl (S)-4-amino-5-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate

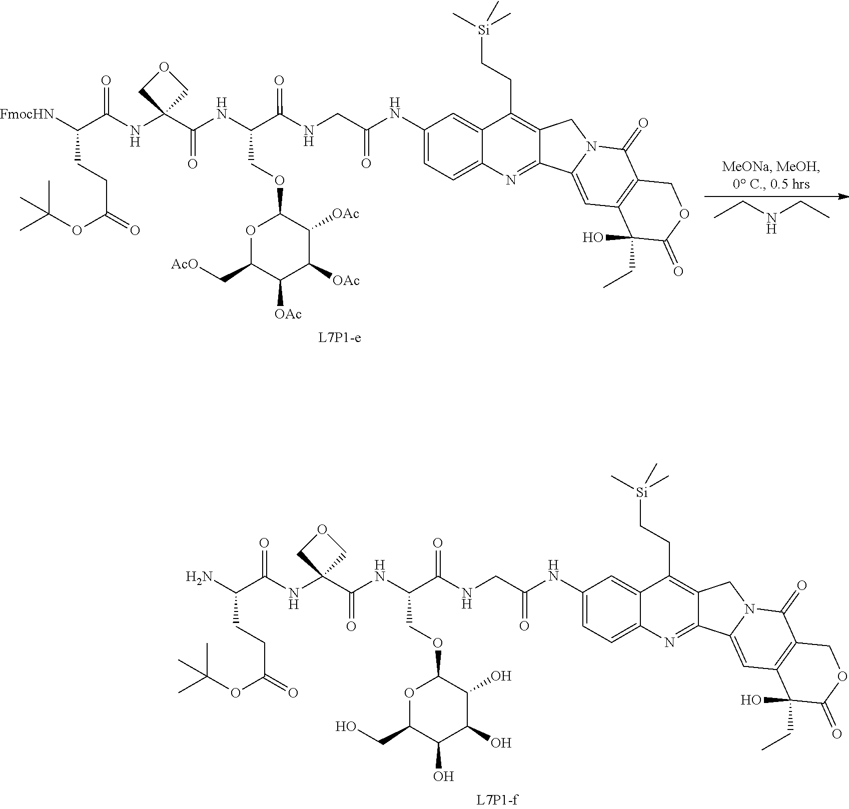

Intermediate L7P1-e ((2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate, 205 mg, 0.14 mmol) was dissolved in 5 mL methanol (CAS RN: 67-56-1) under 0° C. Then sodium methoxide (CAS RN: 124-41-4; 105.12 μL, 5 mol/L in methanol, 0.53 mmol) was added to the reaction mixture and stirred for 2 hours under 0° C. LC-MS was employed to verify the complete conversion of intermediate L7P1-e in the reaction mixture. Then diethylamine (CAS RN: 109-89-7; 73.09 μL, 0.71 mmol) was added to the reaction mixture and stirred for additional 2 hours. 0.1 mol/L HCl solution was added to the reaction mixture to adjust the pH value of the mixture to 5.0 to 7.0. The organic solvent and water in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-f as a yellow solid (tert-butyl (S)-4-amino-5-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate; 110 mg, 0.10 mmol, 71.43% in yield).

LC-MS (m/z, C49H67N7O17Si)=1078.4[M+23]⁺.

¹H NMR (400 MHz, DMSO) δ 10.29 (s, 1H), 9.45 (s, 1H), 8.69 (d, J=2.3 Hz, 1H), 8.39 (t, J=5.8 Hz, 1H), 8.18-8.11 (m, 3H), 7.95 (dd, J=9.2, 2.1 Hz, 1H), 7.85 (d, J=7.7 Hz, 1H), 7.30 (s, 1H), 6.53 (s, 1H), 5.45 (s, 2H), 5.33 (s, 2H), 4.95 (d, J=6.6 Hz, 1H), 4.91 (d, J=6.6 Hz, 1H), 4.63-4.60 (m, 2H), 4.52 (d, J=6.7 Hz, 1H), 4.18 (d, J=7.5 Hz, 1H), 4.06-4.00 (m, 3H), 3.95 (s, 1H), 3.80-3.72 (m, 1H), 3.68 (s, 1H), 3.58-3.51 (m, 4H), 3.38 (d, J=7.5 Hz, 4H), 3.32 (d, J=10.5 Hz, 2H), 3.07 (s, 2H), 2.41 (t, J=8.1 Hz, 2H), 2.10-2.01 (m, 2H), 1.92-1.85 (m, 2H), 1.41 (s, 9H), 0.95 (t, J=8.8 Hz, 2H), 0.90 (t, J=7.3 Hz, 3H), 0.19 (s, 9H).

### Intermediate L7P1-g: tert-butyl (S)-18-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-1-(9H-fluoren-9-yl)-3,16-dioxo-2,7,10,13-tetraoxa-4,17-diazahenicosan-21-oate

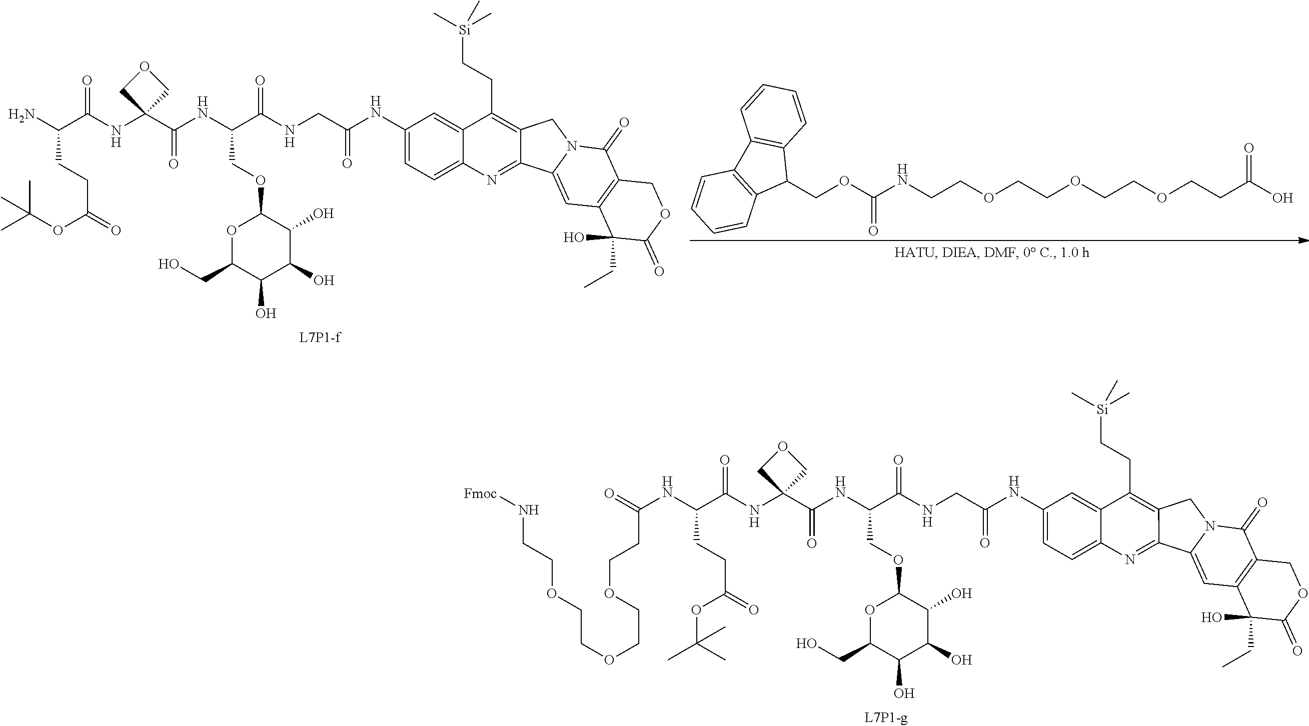

N-Fmoc-12-amino-4,7,10-trioxadodecanoic acid (CAS RN: 867062-95-1; 55.53 mg, 0.13 mmol) was dissolved in 5 ml DMF (CAS RN: 68-12-2) under 0° C. The reaction mixture was then added with HATU (CAS RN: 148893-10-1; 59.51 mg, 0.16 mmol) and DIPEA (CAS RN: 7087-68-5; 26.97 mg, 0.21 mmol) and left stirring for 30 minutes under 0° C. Intermediate L7P1-f (tert-butyl (S)-4-amino-5-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate, 110 mg, 0.10 mmol) was added to the reaction mixture and the reaction mixture was left stirring for additional 30 minutes under 0° C. LC-MS was employed to verify the complete conversion of intermediate L7P1-f in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-g as a yellow solid (tert-butyl (S)-18-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-1-(9H-fluoren-9-yl)-3,16-dioxo-2,7,10,13-tetraoxa-4,17-diazahenicosan-21-oate, 130 mg, 0.09 mmol, 87.85% in yield).

LC-MS (m/z, C73H94N8O23Si)=1479.2 [M+H]⁺.

¹H NMR (400 MHz, DMSO) δ 10.17 (s, 1H), 9.17 (s, 1H), 8.71 (s, 1H), 8.24 (d, J=7.0 Hz, 2H), 8.13 (d, J=9.1 Hz, 1H), 7.97 (d, J=9.3 Hz, 1H), 7.90 (t, J=8.2 Hz, 3H), 7.74-7.67 (m, 3H), 7.42 (q, J=7.4 Hz, 3H), 7.37-7.29 (m, 5H), 5.45 (s, 2H), 5.31 (s, 2H), 4.90 (d, J=6.5 Hz, 1H), 4.83 (d, J=6.5 Hz, 1H), 4.53-4.51 (m, 2H), 4.29 (d, J=6.6 Hz, 2H), 4.20 (t, J=7.3 Hz, 4H), 4.03 (d, J=5.9 Hz, 2H), 3.98 (t, J=5.3 Hz, 2H), 3.79 (d, J=5.9 Hz, 2H), 3.67 (d, J=3.0 Hz, 2H), 3.63-3.55 (m, 4H), 3.53-3.46 (m, 14H), 3.40 (t, J=6.4 Hz, 4H), 3.14 (t, J=5.7 Hz, 2H), 3.06 (s, 2H), 1.94-1.82 (m, 3H), 1.39 (s, 9H), 0.98-0.92 (m, 2H), 0.89 (t, J=7.3 Hz, 3H), 0.18 (s, 9H).

### Intermediate L7P1-h: tert-butyl (S)-1-amino-14-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate

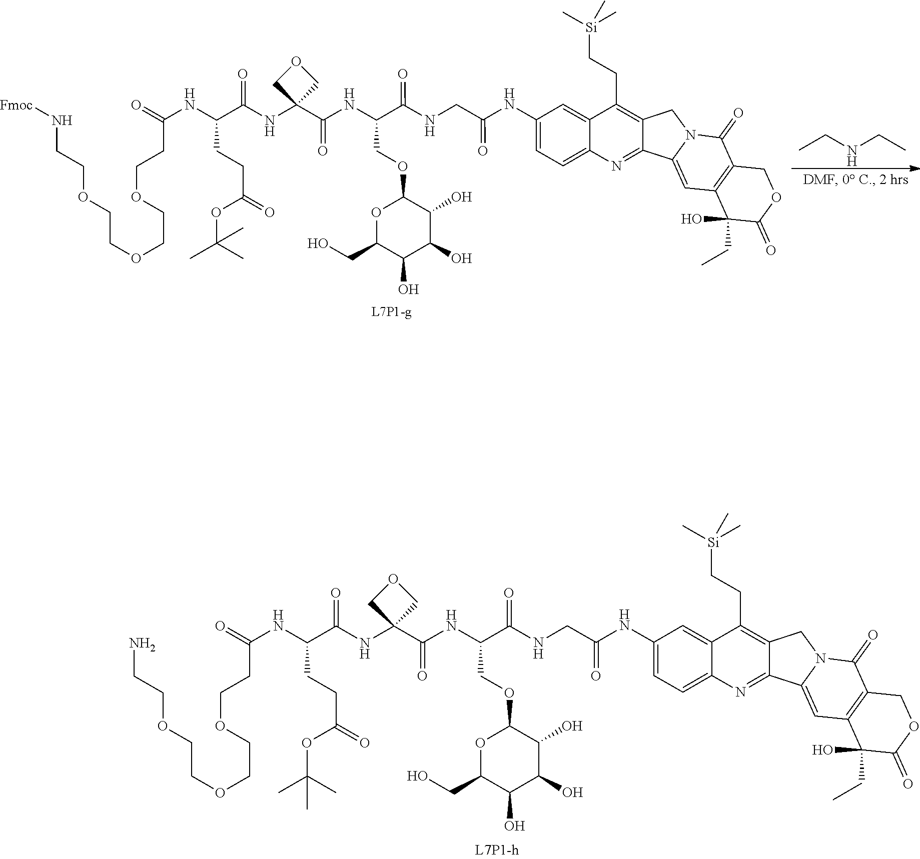

Intermediate L7P1-g (tert-butyl (S)-18-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-1-(9H-fluoren-9-yl)-3,16-dioxo-2,7,10,13-tetraoxa-4,17-diazahenicosan-21-oate, 170 mg, 0.11 mmol) was dissolved in 5 mL DMF (CAS RN: 68-12-2) under 0° C. Then diethylamine (CAS RN: 109-89-7; 0.12 mL, 1.15 mmol) was added to the reaction mixture and stirred for 2 hours under 0° C. The organic solvent and excess amount of diethylamine in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-h as a light yellow solid (tert-butyl (S)-1-amino-14-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate; 140 mg, 0.11 mmol, 96.91% in yield).

LC-MS (m/z, C58H84N8O21Si)=1256.4 [M]⁺& 629[(M+2)/2]⁺.

¹H NMR (400 MHz, DMSO) δ 10.19 (s, 1H), 9.17 (s, 1H), 8.71 (d, J=2.2 Hz, 1H), 8.27 (d, J=6.1 Hz, 1H), 8.24 (d, J=6.4 Hz, 1H), 8.14 (d, J=9.1 Hz, 1H), 7.98 (d, J=9.1 Hz, 1H), 7.72 (d, J=9.8 Hz, 3H), 7.30 (s, 1H), 6.53 (s, 1H), 5.45 (s, 2H), 5.33 (s, 2H), 4.90 (d, J=6.6 Hz, 1H), 4.84 (d, J=6.4 Hz, 1H), 4.56-4.49 (m, 3H), 4.19 (d, J=7.1 Hz, 2H), 4.03 (d, J=5.9 Hz, 2H), 4.01-3.96 (m, 1H), 3.80-3.75 (m, 1H), 3.67-3.65 (m, 2H), 3.62-3.55 (m, 12H), 3.40 (t, J=6.5 Hz, 2H), 3.36-3.32 (m, 2H), 3.07 (s, 2H), 3.01-2.97 (m, 2H), 2.47-2.38 (m, 4H), 2.32-2.26 (m, 3H), 1.96-1.80 (m, 4H), 1.40 (s, 9H), 0.94 (d, J=9.4 Hz, 2H), 0.90 (t, J=7.3 Hz, 3H), 0.19 (s, 9H).

### Intermediate L7P1-i: tert-butyl (S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate

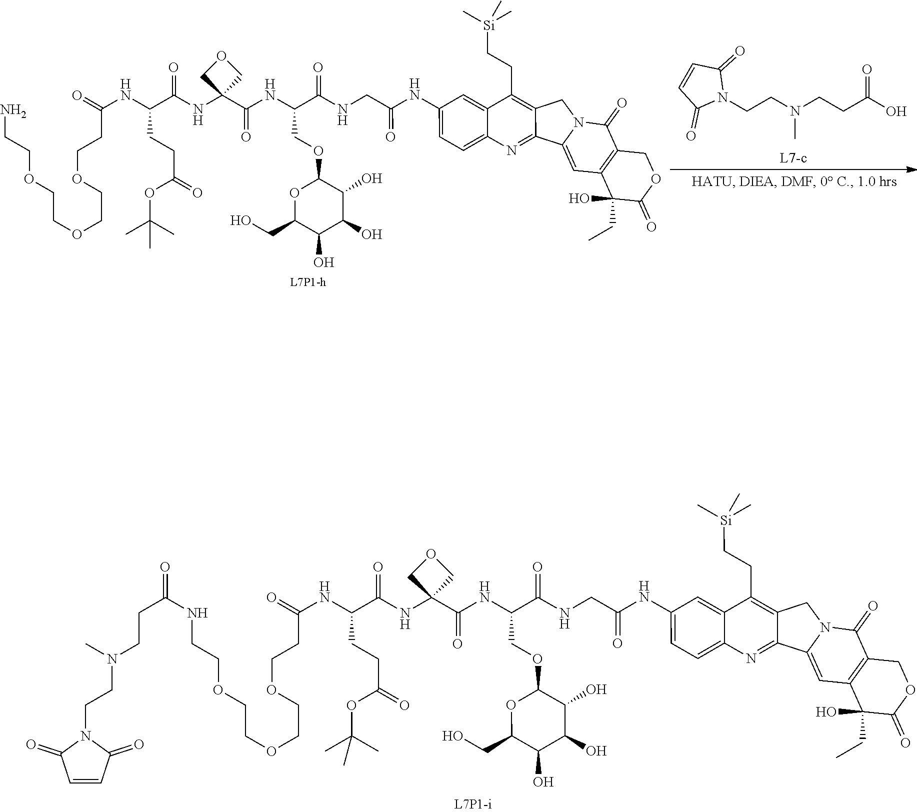

Intermediate L7-c (3-((2-(2,5-dioxo-2,5-dihydro-1H-pyrrol-1-yl)ethyl)(methyl) amino)propanoic acid; 51.28 mg, 0.23 mmol) was dissolved in 5 ml DMF (CAS RN: 68-12-2) under 0° C. The reaction mixture was then added with HATU (CAS RN: 148893-10-1; 28.73 mg, 0.08 mmol) and DIPEA (CAS RN: 7087-68-5; 9.76 mg, 0.08 mmol) and left stirring for 30 minutes under 0° C. Intermediate L7P1-h (tert-butyl (S)-1-amino-14-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate, 95 mg, 0.08 mmol) was added to the reaction mixture and the reaction mixture was left stirring for additional 30 minutes under 0° C. LC-MS was employed to verify the complete conversion of intermediate L7P1-h in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P1-i as a yellow solid (tert-butyl (S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate; 80 mg, 0.05 mmol, 68.40% in yield).

LC-MS (m/z, C69H98N10O23Si)=733.4 [(M+2)/2]⁺.

¹H NMR (400 MHz, DMSO) δ 10.18 (s, 1H), 9.27 (s, 1H), 9.17 (s, 1H), 8.71 (s, 1H), 8.25 (t, J=8.7 Hz, 3H), 8.14 (d, J=9.1 Hz, 1H), 7.97 (d, J=9.3 Hz, 1H), 7.72 (d, J=7.2 Hz, 1H), 7.30 (s, 1H), 7.12 (s, 2H), 6.52 (s, 1H), 5.45 (s, 2H), 5.33 (s, 2H), 4.90 (d, J=6.6 Hz, 1H), 4.84 (d, J=6.5 Hz, 1H), 4.56-4.49 (m, 3H), 4.19 (d, J=7.1 Hz, 2H), 4.03 (d, J=5.8 Hz, 2H), 3.97 (d, J=6.0 Hz, 1H), 3.79 (s, 2H), 3.64-3.60 (m, 2H), 3.57-3.54 (m, 2H), 3.51-3.49 (m, 12H), 3.42 (d, J=5.6 Hz, 6H), 3.37-3.32 (m, 4H), 3.24 (t, J=5.9 Hz, 2H), 3.07 (s, 2H), 2.82 (s, 3H), 2.58 (s, 1H), 2.47-2.38 (m, 4H), 2.32-2.26 (m, 3H), 1.94-1.85 (m, 4H), 1.39 (s, 9H), 0.97-0.95 (m, 2H), 0.90 (t, J=7.3 Hz, 3H), 0.19 (s, 9H).

### Linker-payload L7P1: (S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oic acid

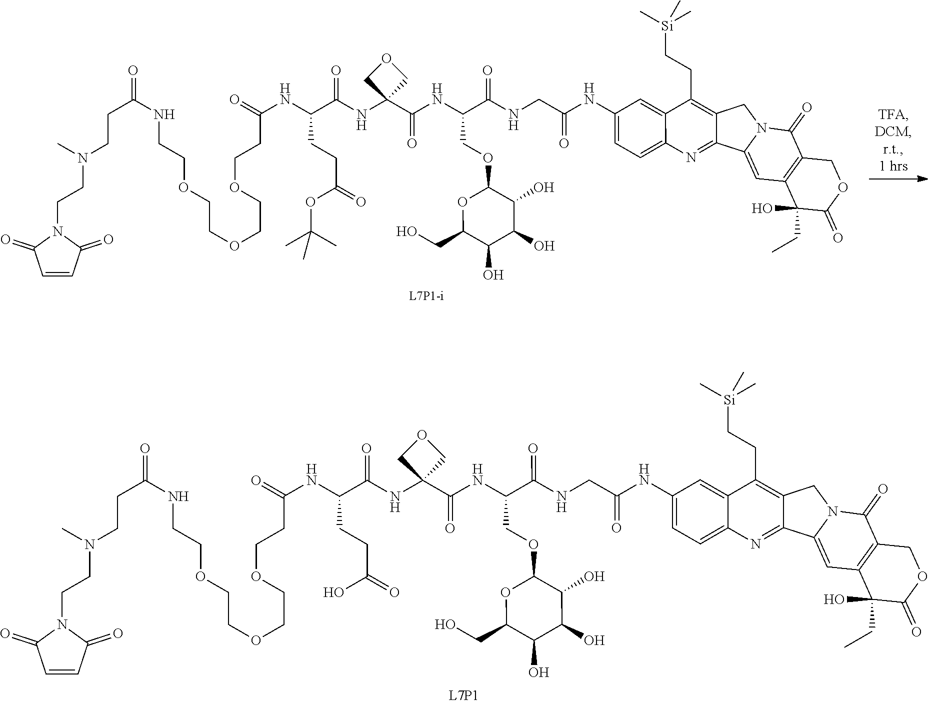

Intermediate L7P1-i (tert-butyl (S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate; 80 mg, 0.05 mmol) was dissolved in DCM (CAS RN: 75-09-2, 5 mL) at room temperature. Then, trifluoroacetic acid (CAS RN: 76-05-1; 2 mL, 26.14 mmol) was added to the reaction mixture and stirred for 30 minutes at room temperature. LC-MS was employed to verify the complete consumption of intermediate L7P1-i in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the linker-payload L7P1 as a yellow solid ((S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oic acid; 15 mg, 0.01 mmol, 19.50% in yield after purification).

LC-MS (m/z, C64H88N10O24Si)=1431.6 [M+23]⁺& 705[(M+2)/2]⁺.

¹H NMR (400 MHz, DMSO) δ 12.16 (s, 1H), 10.19 (s, 1H), 9.28 (d, J=14.5 Hz, 1H), 9.18 (s, 1H), 8.72 (d, J=2.2 Hz, 1H), 8.29-8.22 (m, 2H), 8.14 (d, J=9.1 Hz, 1H), 7.98 (dd, J=9.2, 2.2 Hz, 1H), 7.74 (d, J=7.2 Hz, 1H), 7.31 (s, 1H), 7.12 (s, 2H), 6.53 (s, 1H), 5.45 (s, 2H), 5.33 (s, 2H), 4.91 (d, J=6.5 Hz, 1H), 4.82 (d, J=6.5 Hz, 1H), 4.55 (d, J=6.6 Hz, 2H), 4.50 (d, J=6.4 Hz, 1H), 4.24-4.16 (m, 2H), 4.03 (d, J=5.8 Hz, 2H), 4.04-3.96 (m, 1H), 3.79 (s, 2H), 3.67 (d, J=3.2 Hz, 1H), 3.65-3.59 (m, 2H), 3.59-3.53 (m, 2H), 3.51-3.39 (d, J=5.8 Hz, 8H), 3.41 (d, J=5.7 Hz, 6H), 3.36 (s, 12H), 3.26-3.22 (m, 2H), 3.11-3.04 (m, 2H), 2.82 (s, 3H), 2.57 (d, J=9.7 Hz, 2H), 2.47-2.38 (m, 2H), 2.35-2.28 (m, 2H), 0.95 (t, J=9.1 Hz, 2H), 0.90 (t, J=7.3 Hz, 3H), 0.19 (s, 9H).

### Example 4: Linker-payload L2P2

### Intermediate L2P2-a: (9H-fluoren-9-yl)methyl ((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamate

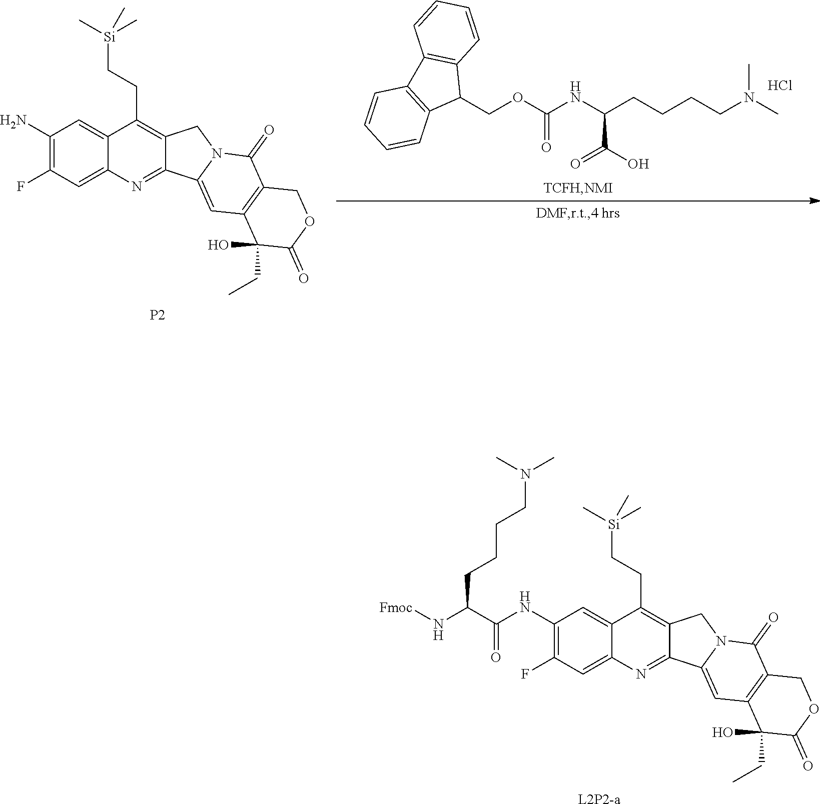

Payload P2 ((S)-9-amino-4-ethyl-8-fluoro-4-hydroxy-11-(2-(trimethylsilyl)ethyl)-1,12-dihydro-14H-pyrano[3′,4′:6,7]indolizino[1,2-b]quinoline-3,14(4H)-dione; 400 mg, 0.83 mmol) was dissolved in 8 ml DMF (CAS RN: 68-12-2) at room temperature. The reaction mixture was then added with Fmoc-N′,N′-dimethyl-L-lysine hydrochloride (CAS RN: 252049-10-8; 400 mg, 0.92 mmol), TCFH (CAS RN: 94790-35-9; 466 mg, 1.66 mmol), and 1-methylimidazole (CAS RN: 616-47-7; 102 mg, 1.24 mmol) and left stirring for 4 hours at room temperature. LC-MS was employed to verify the complete conversion of payload P2 in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L2P2-a as a brownsolid ((9H-fluoren-9-yl)methyl ((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamate, 150 mg, 0.17 mmol, 21.0% in yield).

LC-MS (m/z, C48H54FN5O7Si)=860.3 [M+H]⁺.

¹H NMR (400 MHz, DMSO-d6) δ 10.24 (s, 1H), 8.92 (m, 1H), 8.32 (s, 1H), 8.04 (m, 1H), 7.91 (m, 2H), 7.76 (m, 2H), 7.43 (m, 2H), 7.37-7.28 (m, 3H), 6.55 (s, 1H), 5.45 (s, 2H), 5.33 (s, 2H), 4.44 (s, 1H), 4.36-4.25 (m, 3H), 3.07 (m, 2H), 2.28 (s, 2H), 2.17 (s, 6H), 1.92-1.77 (m, 4H), 1.47 (s, 4H), 0.91-0.87 (m, 5H), 0.15 (s, 9H).

### Intermediate L2P2-b: (S)-2-amino-6-(dimethylamino)-N—((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)hexanamide

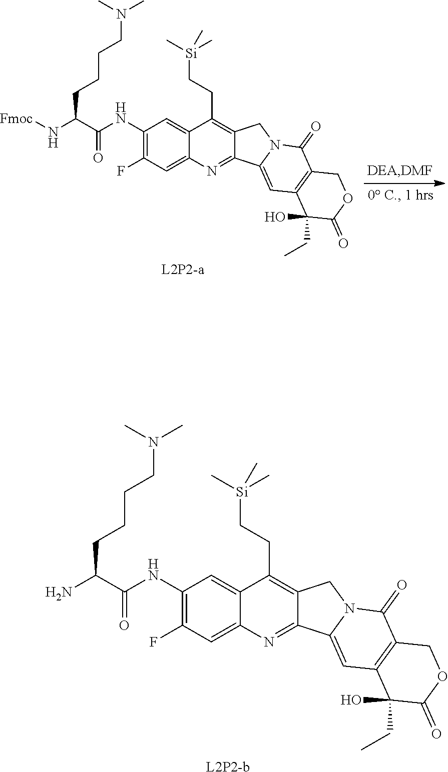

Intermediate L2P2-a ((9H-fluoren-9-yl)methyl ((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamate, 480 mg, 0.56 mmol) was dissolved in 6 mL DMF (CAS RN: 68-12-2) under 0° C. Then diethylamine (CAS RN: 109-89-7; 400 mg, 5.47 mmol) was added to the reaction mixture and stirred for 1 hour under 0° C. The organic solvent and excess amount of diethylamine in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to afford the intermediate L2P2-b as a white solid ((S)-2-amino-6-(dimethylamino)-N—((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)hexanamide; 210 mg, 0.33 mmol, 58.93% in yield).

LC-MS (m/z, C33H44FN5O5Si)=638.4 [M+H]⁺.

¹H NMR (400 MHz, DMSO-d₆) δ 9.09 (m, 1H), 8.29 (s, 1H), 8.05 (m, 1H), 7.31 (s, 1H), 6.55 (bs, 1H), 5.45 (s, 2H), 5.33 (s, 2H), 3.56-3.52 (m, 1H), 3.07 (m, 2H), 2.30 (m, 2H), 2.19 (s, 6H), 1.94-1.75 (m, 4H), 1.45 (s, 4H), 0.93-0.87 (m, 5H), 0.19 (s, 9H).

### Intermediate L2P2-c: tert-butyl (S)-4-((((9H-fluoren-9-yl)methoxy)carbonyl)amino)-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate

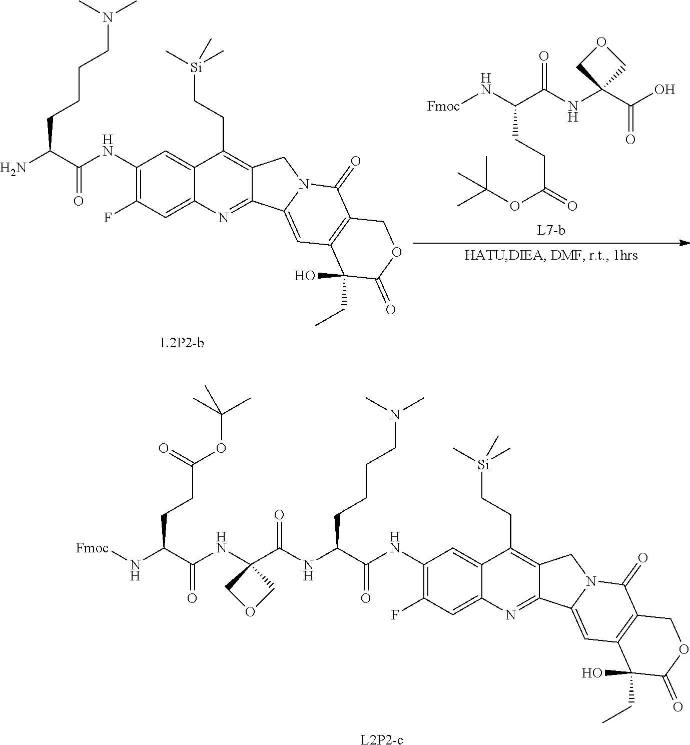

Intermediate L7-b ((S)-3-(2-((((9H-fluoren-9-yl)methoxy)carbonyl)amino)-5-(tert-butoxy)-5-oxopentanamido)oxetane-3-carboxylic acid; 222 mg, 0.42 mmol) was dissolved in 10 ml DMF (CAS RN: 68-12-2) under 0° C. The reaction mixture was then added with HATU (CAS RN: 148893-10-1; 140 mg, 0.37 mmol) and DIPEA (CAS RN: 7087-68-5; 109 mg, 0.84 mmol) and left stirring for 30 minutes under 0° C. Intermediate L2P2-b ((S)-2-amino-6-(dimethylamino)-N—((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)hexanamide, 200 mg, 0.28 mmol) was added to the reaction mixture and the reaction mixture was left stirring for additional 30 minutes under 0° C. LC-MS was employed to verify the complete conversion of intermediate L2P2-b in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L2P2-c as a white solid (tert-butyl (S)-4-((((9H-fluoren-9-yl)methoxy)carbonyl)amino)-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate, 220 mg, 0.19 mmol, 67.90% in yield).

LC-MS (m/z, C61H74FN7O12Si)=1145.4[M+H]⁺& 573[(M+2)/2]⁺.

¹H NMR (400 MHz, DMSO-d₆) δ 10.20 (d, J=32.8 Hz, 1H), 8.84 (dd, J=28.0, 8.7 Hz, 1H), 8.03 (dd, J=12.0, 7.5 Hz, 1H), 7.92-7.84 (m, 4H), 7.70 (d, J=7.6 Hz, 1H), 7.48-7.37 (m, 3H), 7.32 (s, 2H), 6.54 (s, 1H), 6.30 (s, 1H), 5.45 (s, 2H), 5.32 (d, J=6.4 Hz, 2H), 4.97 (dd, J=12.8, 6.4 Hz, 1H), 4.73 (d, J=7.2 Hz, 1H), 4.63 (t, J=6.4 Hz, 2H), 4.53-4.38 (m, 2H), 4.26 (dd, J=13.2, 8.4 Hz, 2H), 2.36-2.26 (m, 5H), 2.13 (s, 6H), 2.04 (s, 4H), 1.87 (dd, J=17.2, 9.2 Hz, 6H), 1.40 (d, J=6.4 Hz, 13H), 0.91-0.89 (m, 5H), 0.15 (s, 9H).

### Intermediate L2P2-d: tert-butyl (S)-4-amino-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate

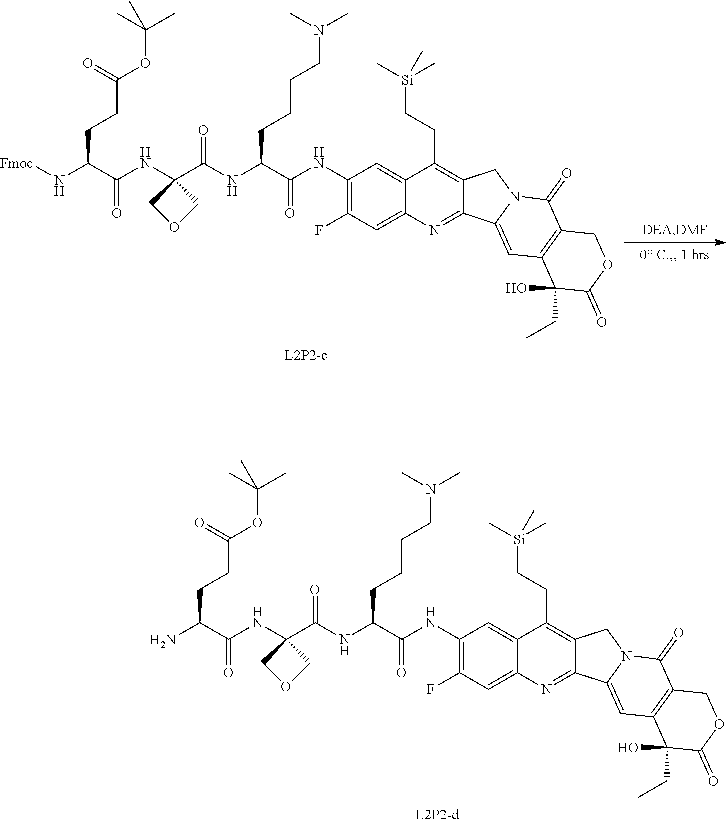

Intermediate L2P2-c (tert-butyl (S)-4-((((9H-fluoren-9-yl)methoxy)carbonyl)amino)-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate; 220 mg, 0.19 mmol) was dissolved in 6 mL DMF (CAS RN: 68-12-2) under 0° C. Then diethylamine (CAS RN: 109-89-7; 140 mg, 1.91 mmol) was added to the reaction mixture and stirred for 1 hour under 0° C. The organic solvent and excess amount of diethylamine in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to afford the intermediate L2P2-d as a white solid (tert-butyl (S)-4-amino-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate; 170 mg, 0.18 mmol, 97.0% in yield).

LC-MS (m/z, C46H64FN7O10Si)=922.4[M+H]⁺& 461.8[(M+2)/2]⁺.

¹H NMR (400 MHz, DMSO-d₆) δ10.25 (s, 1H), 8.87 (d, J=8.4 Hz, 1H), 8.03 (d, J=12.0 Hz, 1H), 7.90 (d, J=7.8 Hz, 1H), 7.32 (s, 1H), 5.45 (s, 2H), 5.33 (s, 2H), 4.99 (d, J=6.4 Hz, 1H), 4.73 (d, J=6.4 Hz, 1H), 4.64 (d, J=6.8 Hz, 2H), 4.46 (d, J=6.4 Hz, 1H), 3.27-3.23 (m, 2H), 3.07 (s, 3H), 2.43-2.21 (m, 6H), 2.14 (s, 6H), 1.91-1.86 (m, 5H), 1.39 (s, 9H), 0.91-0.87 (m, 5H), 0.17 (s, 9H).

### Intermediate L2P2-e: tert-butyl (S)-1-amino-14-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-12-oxo-3,6,9-trioxa-13-azaheptadecan-17-oate

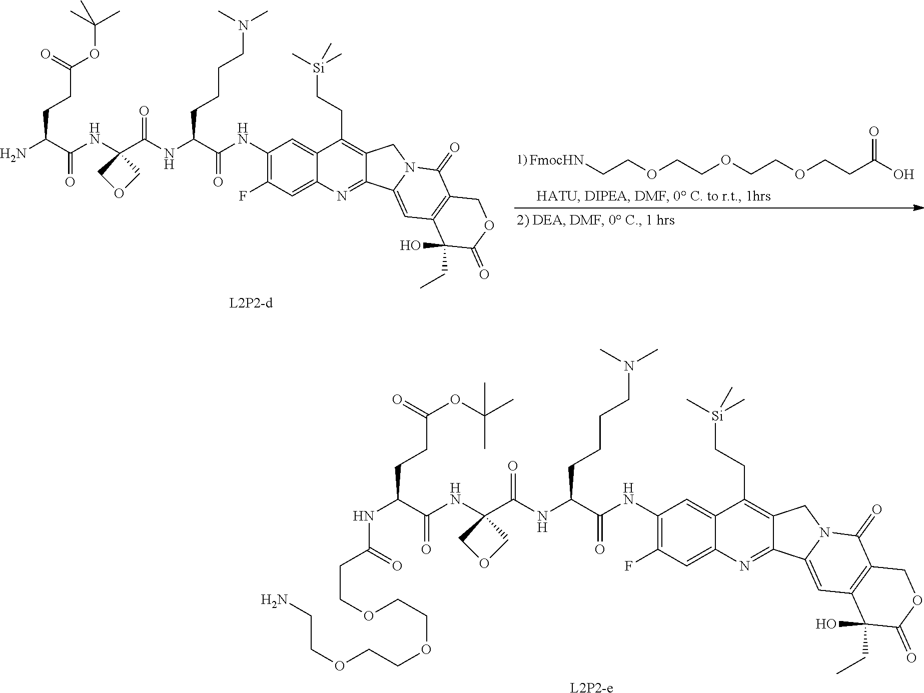

N-Fmoc-12-amino-4,7,10-trioxadodecanoic acid (CAS RN: 867062-95-1; 112 mg, 0.25 mmol) was dissolved in 5 ml DMF (CAS RN: 68-12-2) under 0° C. The reaction mixture was then added with HATU (CAS RN: 148893-10-1; 72 mg, 0.19 mmol) and DIPEA (CAS RN: 7087-68-5; 50 mg, 0.39 mmol) and left stirring for 30 minutes under 0° C. Intermediate L2P2-d (tert-butyl (S)-4-amino-5-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)amino)-5-oxopentanoate, 110 mg, 0.12 mmol) was added to the reaction mixture. The reaction mixture was warmed to room temperature and left stirring for additional 1 hour. LC-MS was employed to verify the complete conversion of intermediate L2P2-d in the reaction mixture. Then diethylamine (CAS RN: 109-89-7; 175 mg, 2.39 mmol) was added to the reaction mixture and stirred for 1 hour at room temperature. In the end, 0.2 mL formic acid (CAS RN: 64-18-6) was added to the reaction mixture to neutralize the excess diethylamine. The organic solvent in the reaction mixture was reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L2P2-e as a white solid (tert-butyl (S)-1-amino-14-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-12-oxo-3,6,9-trioxa-13-azaheptadecan-17-oate; 100 mg, 0.09 mmol, 74.49% in yield).

LC-MS (m/z, C55H81FN8O14Si)=1147.6[M+23]⁺.

¹H NMR (400 MHz, DMSO-d6) δ 10.11 (s, 1H), 9.43 (s, 1H), 9.12 (s, 1H), 8.85 (d, J=8.4 Hz, 1H), 8.31 (d, J=6.4 Hz, 2H), 8.04 (d, J=12.0 Hz, 1H), 7.77 (d, J=7.8 Hz, 1H), 7.32 (s, 1H), 7.11 (s, 2H), 6.55 (s, 1H), 5.46 (s, 2H), 5.34 (s, 2H), 4.93 (d, J=6.4 Hz, 1H), 4.77 (d, J=6.4 Hz, 1H), 4.69 (s, 1H), 4.59 (d, J=6.8 Hz, 1H), 4.49 (d, J=6.4 Hz, 1H), 4.15 (d, J=7.4 Hz, 1H), 3.83-3.60 (m, 6H), 3.50 (d, J=3.6 Hz, 12H), 3.23 (d, J=6.0 Hz, 3H), 3.05 (s, 5H), 2.79 (d, J=3.2 Hz, 10H), 2.41-2.27 (m, 4H), 1.92-1.85 (m, 7H), 1.64 (s, 2H), 1.39 (s, 9H), 0.91-0.87 (m, 5H), 0.17 (s, 9H).

### Intermediate L2P2-f: tert-butyl (S)-21-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-1-(2,5-dioxo-2,5-dihydro-1H-pyrrol-1-yl)-3-methyl-6,19-dioxo-10,13,16-trioxa-3,7,20-triazatetracosan-24-oate

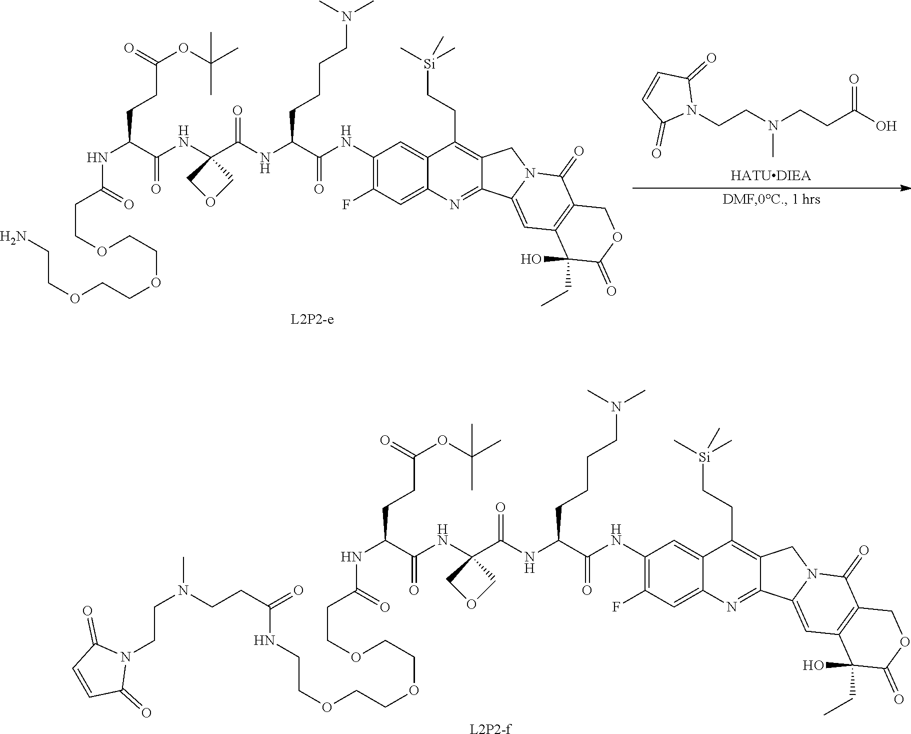

Intermediate L7-c (3-((2-(2,5-dioxo-2,5-dihydro-1H-pyrrol-1-yl)ethyl)(methyl) amino)propanoic acid; 61 mg, 0.27 mmol) was dissolved in 3 ml DMF (CAS RN: 68-12-2) under 0° C. The reaction mixture was then added with HATU (CAS RN: 148893-10-1; 51 mg, 0.13 mmol) and DIPEA (CAS RN: 7087-68-5; 40 mg, 0.31 mmol) and left stirring for 30 minutes under 0° C. Intermediate L2P2-e (tert-butyl (S)-1-amino-14-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-12-oxo-3,6,9-trioxa-13-azaheptadecan-17-oate; 100 mg, 0.09 mmol) was added to the reaction mixture and the reaction mixture was left stirring for additional 30 minutes under 0° C. LC-MS was employed to verify the complete conversion of intermediate L2P2-e in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L2P2-f as a white solid (tert-butyl (S)-21-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-1-(2,5-dioxo-2,5-dihydro-1H-pyrrol-1-yl)-3-methyl-6,19-dioxo-10,13,16-trioxa-3,7,20-triazatetracosan-24-oate; 90 mg, 0.067 mmol, 74.44% in yield).

LC-MS (m/z, C65H93FN10O17Si)=1356.6[M+23]⁺& [(M+2)/2]⁺.

¹H NMR (400 MHz, DMSO-d6) δ 8.83 (d, J=8.0 Hz, 1H), 8.03 (d, J=12.0 Hz, 1H), 7.32 (s, 1H), 5.45 (s, 2H), 5.34 (s, 2H), 4.92 (d, J=6.4 Hz, 1H), 4.74 (d, J=6.4 Hz, 1H), 4.59 (d, J=6.8 Hz, 2H), 4.48 (d, J=6.4 Hz, 1H), 4.15 (s, 2H), 3.64 (d, J=6.8 Hz, 4H), 3.54 (s, 9H), 3.09 (s, 4H), 2.88 (s, 5H), 2.22 (d, J=6.8 Hz, 3H), 2.13 (s, 7H), 1.91-1.83 (m, 8H), 1.40 (s, 15H), 0.91-0.87 (m, 5H), 0.17 (s, 9H).

### Linker-payload L2P2: (S)-21-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-1-(2,5-dioxo-2,5-dihydro-1H-pyrrol-1-yl)-3-methyl-6,19-dioxo-10,13,16-trioxa-3,7,20-triazatetracosan-24-oic acid

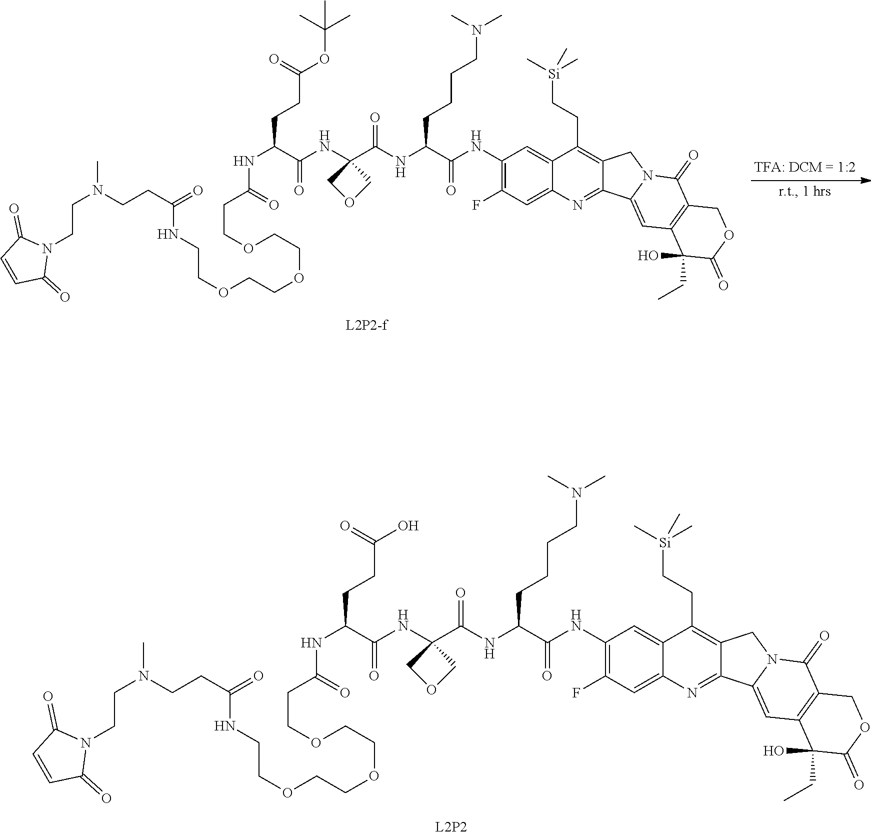

Intermediate L2P2-f (tert-butyl (S)-21-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-1-oxohexan-2-yl)carbamoyl)oxetan-3-yl)carbamoyl)-1-(2,5-dioxo-2,5-dihydro-1H-pyrrol-1-yl)-3-methyl-6,19-dioxo-10,13,16-trioxa-3,7,20-triazatetracosan-24-oate; 90 mg, 0.067 mmol) was dissolved in DCM (CAS RN: 75-09-2, 4 mL) at room temperature. Then, trifluoroacetic acid (CAS RN: 76-05-1; 2 mL, 26.14 mmol) was added to the reaction mixture and stirred for 30 minutes at room temperature. LC-MS was employed to verify the complete consumption of intermediate L2P2-f in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the linker-payload L2P2 as a white solid ((S)-1-(2,5-dioxo-2,5-dihydro-1H-pyrrolo-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolazino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetane-3-yl)carbamoyl)-3-methyl-6,19-dioxo-10,13,16-trioxa-3,7,20-triazatetracosan-24-oic acid; 22 mg, 0.017 mmol, 25.70% in yield after purification).

LC-MS (m/z, C61H85FN10O17Si)=639.4[M/2+1]⁺.

¹H NMR (400 MHz, DMSO-d₆) δ12.18 (s, 1H), 10.11 (s, 1H), 9.36 (s, 1H), 8.87 (d, J=8.4 Hz, 1H), 7.32 (s, 1H), 7.10 (s, 2H), 6.54 (s, 1H), 5.46 (s, 2H), 5.34 (s, 2H), 4.95 (d, J=6.4 Hz, 1H), 4.78-4.59 (m, 3H), 4.48 (d, J=6.4 Hz, 1H), 4.14 (d, J=7.1 Hz, 1H), 3.66-3.61 (m, 4H), 3.50 (s, 11H), 3.40 (s, 6H), 3.21 (s, 3H), 3.14-2.98 (m, 6H), 2.78 (s, 8H), 2.39-2.30 (m, 4H), 2.02-1.79 (m, 6H), 1.64 (s, 2H), 1.35 (d, J=14.8 Hz, 2H), 0.97-0.87 (m, 5H), 0.17 (s, 9H).

### Example 5: Linker-Payload L3P2

### Intermediate L3P2-a: tert-butyl (S)-(2-((4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

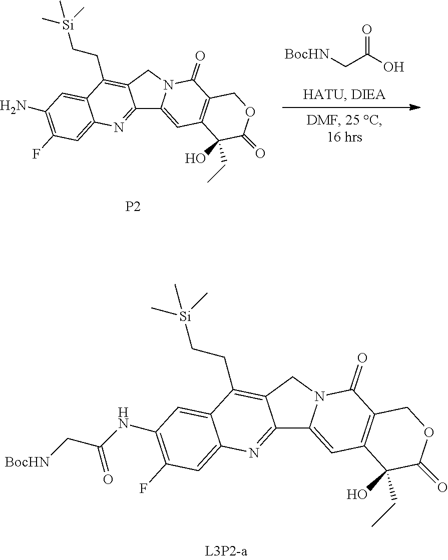 [SMILES: CC[C@@]1(O)C(=O)OCc2c1cc1n(c2=O)Cc2c-1nc1cc(F)c(N)cc1c2CC[Si](C)(C)C ; CC(C)(C)OC(=O)NCC(=O)O ; "P2" ; CC[C@@]1(O)C(=O)OCc2c1cc1n(c2=O)Cc2c-1nc1cc(F)c(NC(=O)CNC(=O)OC(C)(C)C)cc1c2CC[Si](C)(C)C]

Boc-glycine (CAS RN: 4530-20-5; 910 mg, 5.19 mmol) was dissolved in DMF (CAS RN: 68-12-2; 15 ml) at room temperature. The reaction mixture was then added with HATU (CAS RN: 148893-10-1; 1980 mg, 5.21 mmol) and DIPEA (CAS RN: 7087-68-5; 680 mg, 5.26 mmol) and left stirring at room temperature for 0.5 hours. Payload P2 ((S)-9-amino-4-ethyl-8-fluoro-4-hydroxy-11-(2-(trimethylsilyl)ethyl)-1,12-dihydro-14H-pyrano[3′,4′:6,7]indolizino[1,2-b]quinoline-3,14(4H)-dione; 500 mg, 0.93 mmol) was added to the reaction mixture and the reaction mixture was warmed to room temperature and stirred for 16 hours. LC-MS was employed to verify the complete consumption of Payload P2 in the reaction mixture. The organic solvent in the mixture was reduced in vacuo again to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-a as a brown solid (tert-butyl (S)-(2-((4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate, 500 mg, 0.78 mmol, 84.27% in yield).

LC-MS (m/z, C32H39FN4O7Si)=639.2 [M+H]⁺.

¹H NMR (400 MHz, DMSO-d6) δ 10.12 (s, 1H), 9.02 (m, 1H), 8.29 (s, 1H), 8.04 (m, 1H), 7.32 (s, 1H), 6.53 (s, 1H), 5.45 (s, 2H), 5.33 (s, 2H), 3.92 (m, 2H), 3.09-3.04 (m, 2H), 1.93-1.86 (m, 2H), 1.43 (s, 9H), 0.99-0.89 (m, 5H), 0.18 (s, 9H).

### Intermediate L3P2-b: (S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide

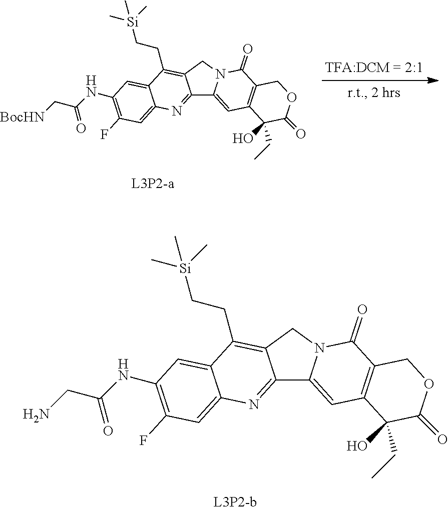 [SMILES: CC[C@@]1(O)C(=O)OCc2c1cc1n(c2=O)Cc2c-1nc1cc(F)c(NC(=O)CNC(=O)OC(C)(C)C)cc1c2CC[Si](C)(C)C ; CC[C@@]1(O)C(=O)OCc2c1cc1n(c2=O)Cc2c-1nc1cc(F)c(NC(=O)CN)cc1c2CC[Si](C)(C)C]

Intermediate L3P2-a (tert-butyl (S)-(2-((4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate, 500 mg, 0.78 mmol) was dissolved in DCM (CAS RN: 75-09-2, 6 mL) at room temperature. Then, trifluoroacetic acid (CAS RN: 76-05-1; 3 mL, 39.2 mmol) was added to the reaction mixture and stirred for 90 minutes at room temperature. LC-MS was employed to verify the complete consumption of intermediate L3P2-a in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-b as a brown solid ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide, 310 mg, 0.57 mmol, 73.08% in yield).

LC-MS (m/z, C27H31FN4O5Si)=539.2[M+H]⁺.

¹H NMR (400 MHz, DMSO-d₆) δ 9.16 (m, 1H), 8.38 (s, 1H), 8.06 (m, 1H), 7.31 (s, 1H), 6.55 (bs, 1H), 5.45 (s, 2H), 5.33 (s, 2H), 3.43 (s, 2H), 3.08-3.04 (m, 2H), 1.92-1.85 (m, 2H), 1.00-0.88 (m, 5H), 0.19 (s, 9H).

### Intermediate L3P2-c: (S)-2-amino-3-((tert-butyldimethylsilyl)oxy)-N-(2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propenamide

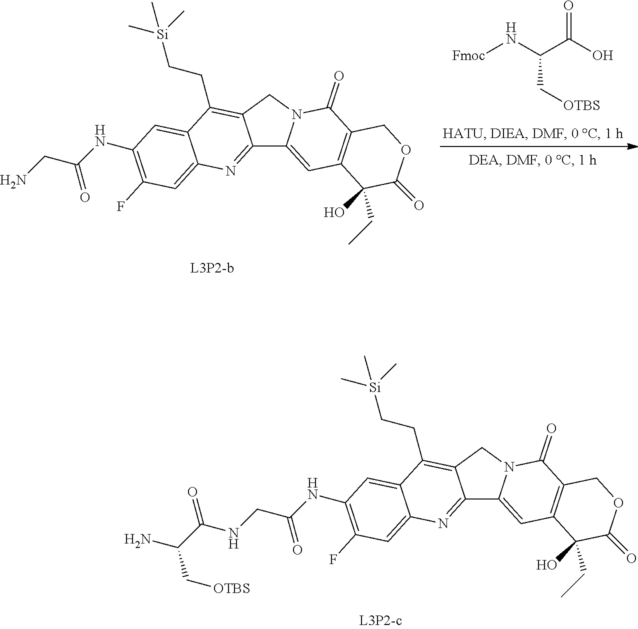

N-(((9H-Fluoren-9-yl)methoxy)carbonyl)-O-(tert-butyldimethylsilyl)-L-serine (CAS RN: 146346-81-8, 350 mg, 0.79 mmol) was dissolved in 10 ml DMF (CAS RN: 68-12-2) under 0° C. The reaction mixture was then added with HATU (CAS RN: 148893-10-1; 302 mg, 0.79 mmol) and DIPEA (CAS RN: 7087-68-5; 205 mg, 1.59 mmol) and left stirring for 30 minutes under 0° C. Intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 310 mg, 0.58 mmol) was added to the reaction mixture. The reaction mixture was left stirring at 0° C. for additional 1 hour. LC-MS was employed to verify the complete conversion of intermediate L3P2-b in the reaction mixture. Then diethylamine (CAS RN: 109-89-7; 470 mg, 6.43 mmol) was added to the reaction mixture and stirred for 1 hour at 0° C. In the end, 0.4 mL formic acid was added to the reaction mixture to neutralize the excess diethylamine. The organic solvent in the reaction mixture was reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-c as a white solid ((S)-2-amino-3-((tert-butyldimethylsilyl)oxy)-N-(2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propanamide; 190 mg, 0.26 mmol, 44.26% in yield).

LC-MS (m/z, C36H50FN5O7Si2)=741.2[M+1]⁺& 370.8[M/2+1]⁺.

¹H NMR (400 MHz, DMSO-d₆) δ 10.22 (s, 1H), 9.01 (m, 1H), 8.38 (s, 1H), 8.03 (m, 1H), 7.32 (s, 1H), 6.54 (s, 1H), 5.45 (s, 2H), 5.33 (s, 2H), 4.11 (s, 2H), 3.71 (m, 2H), 3.40 (m, 1H), 3.07 (m, 2H), 1.92-1.85 (m, 2H), 0.86 (s, 14H), 0.18 (s, 9H), 0.05 (m, 6H).

### Intermediate L3P2-d: tert-butyl (S)-4-amino-5-((3-(((S)-3-((tert-butyldimethylsilyl)oxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate

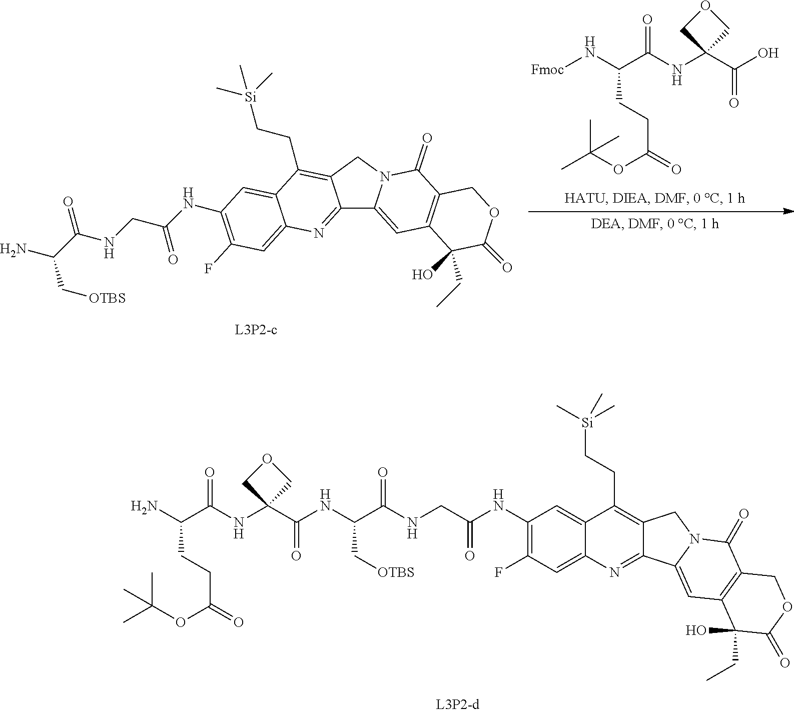

Intermediate L7-b ((S)-3-(2-((((9H-fluoren-9-yl)methoxy)carbonyl)amino)-5-(tert-butoxy)-5-oxopentanamido)oxetane-3-carboxylic acid, 105 mg, 0.20 mmol) was dissolved in 5 ml DMF (CAS RN: 68-12-2) under 0° C. The reaction mixture was then added with HATU (CAS RN: 148893-10-1; 95 mg, 0.25 mmol) and DIPEA (CAS RN: 7087-68-5; 65 mg, 0.50 mmol) and left stirring for 30 minutes under 0° C. Intermediate L3P2-c ((S)-2-amino-3-((tert-butyldimethylsilyl)oxy)-N-(2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propanamide; 140 mg, 0.19 mmol) was added to the reaction mixture. The reaction mixture was warmed to room temperature and left stirring for additional 1 hour. LC-MS was employed to verify the complete conversion of intermediate L3P2-c in the reaction mixture. Then diethylamine (CAS RN: 109-89-7; 150 mg, 2.05 mmol) was added to the reaction mixture and stirred for 1 hour at 0° C. In the end, 0.2 mL formic acid (CAS RN: 64-18-6) was added to the reaction mixture to neutralize the excess diethylamine. The organic solvent in the reaction mixture was reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-d as a white solid (tert-butyl (S)-4-amino-5-((3-(((S)-3-((tert-butyldimethylsilyl)oxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate; 80 mg, 0.09 mmol, 47.36% in yield).

LC-MS (m/z, C49H70FN7O12Si2)=1026.3[M+H]⁺& 1048[M+23]⁺.

¹H NMR (400 MHz, DMSO-d₆) δ 10.20 (s, 1H), 8.99 (d, J=8.4 Hz, 1H), 8.03 (d, J=12.0 Hz, 1H), 7.32 (s, 1H), 6.54 (s, 1H), 5.45 (s, 2H), 5.33 (s, 2H), 4.84 (dd, J=15.6, 6.3 Hz, 1H), 4.55 (d, J=6.4 Hz, 1H), 4.46 (s, 1H), 4.12 (s, 2H), 3.80 (s, 2H), 3.36 (s, 10H), 3.08 (s, 2H), 2.30 (d, J=7.6 Hz, 2H), 1.87-1.84 (m, 2H), 1.39 (s, 9H), 0.99-0.81 (m, 16H), 0.17 (s, 9H).

### Intermediate L3P2-e: tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-12-oxo-3,6,9-trioxa-13-azaheptadecan-17-oate

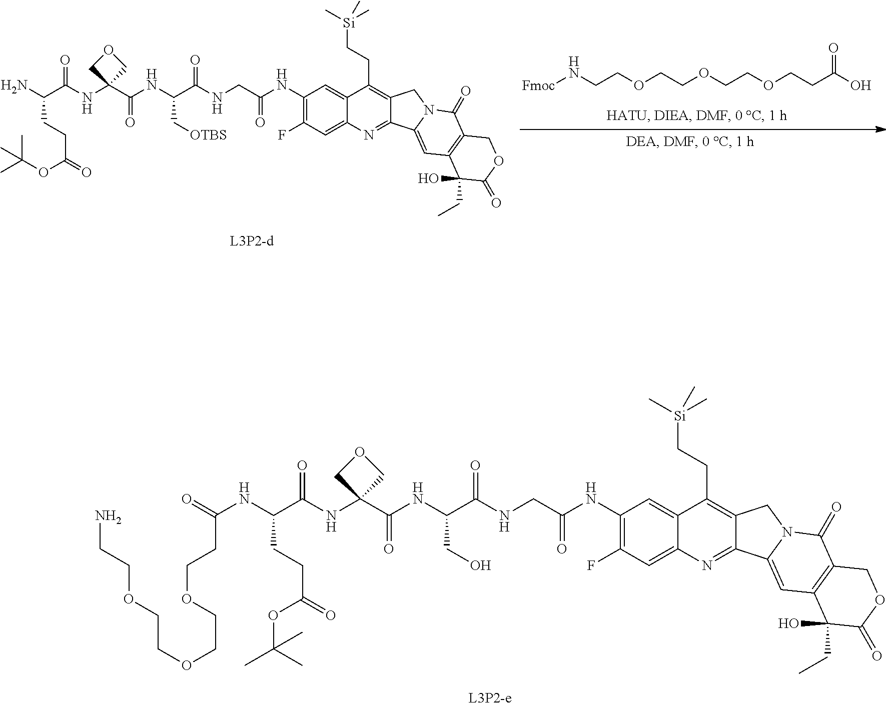

N-Fmoc-12-amino-4,7,10-trioxadodecanoic acid (CAS RN: 867062-95-1; 50 mg, 0.11 mmol) was dissolved in 5 ml DMF (CAS RN: 68-12-2) under 0° C. The reaction mixture was then added with HATU (CAS RN: 148893-10-1; 50 mg, 0.13 mmol) and DIPEA (CAS RN: 7087-68-5; 34 mg, 0.26 mmol) and left stirring for 30 minutes under 0° C. Intermediate L3P2-d (tert-butyl (S)-4-amino-5-((3-(((S)-3-((tert-butyldimethylsilyl)oxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate, 80 mg, 0.08 mmol) was added to the reaction mixture. The reaction mixture was left stirring at 0° C. for additional 0.5 hours. LC-MS was employed to verify the complete conversion of intermediate L3P2-d in the reaction mixture. Then diethylamine (CAS RN: 109-89-7; 100 mg, 1.37 mmol) was added to the reaction mixture and stirred for 1 hour. In the end, 0.1 mL formic acid (CAS RN: 64-18-6) was added to the reaction mixture to neutralize the excess diethylamine. The organic solvent in the reaction mixture was reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-e as a white solid (tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-12-oxo-3,6,9-trioxa-13-azaheptadecan-17-oate; 60 mg, 0.051 mmol, 67.44% in yield).

LC-MS (m/z, C52H73FN8O16Si)=1135.4[M+23]⁺& 557.4 [M/2+1]⁺.

¹H NMR (400 MHz, DMSO-d6) δ9.34 (s, 1H), 8.95 (d, J=8.4 Hz, 1H), 8.49 (s, 1H), 8.03 (d, J=12.0 Hz, 1H), 7.65 (s, 1H), 7.32 (s, 1H), 5.45 (s, 2H), 5.33 (s, 2H), 4.83 (dd, J=42.9, 6.4 Hz, 2H), 4.51 (dd, J=22.4, 6.4 Hz, 2H), 4.34 (d, J=6.9 Hz, 1H), 4.12 (d, J=18.8 Hz, 4H), 3.71-3.48 (m, 26H), 3.08 (s, 3H), 2.28 (d, J=9.6 Hz, 2H), 1.94-1.85 (m, 5H), 0.94-0.87 (m, 5H), 0.18 (s, 9H).

### Intermediate L3P2-f: tert-butyl (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-3-methyl-6,19-dioxo-10,13,16-trioxa-3,7,20-triazatetracosan-24-oate

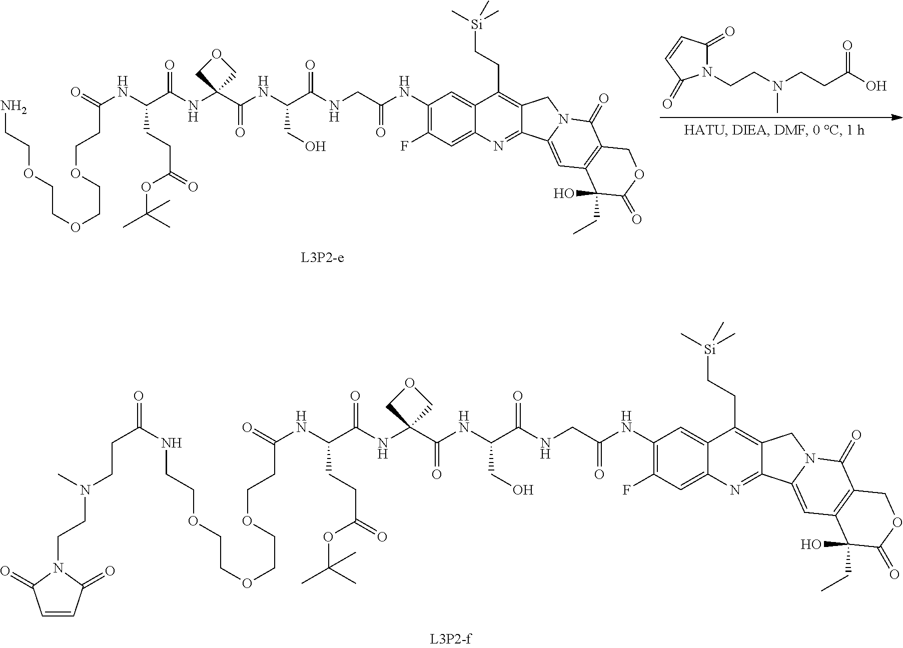

Intermediate L7-c (3-((2-(2,5-dioxo-2,5-dihydro-1H-pyrrol-1-yl)ethyl)(methyl) amino)propanoic acid; 37 mg, 0.16 mmol) was dissolved in 3 ml DMF (CAS RN: 68-12-2) under 0° C. The reaction mixture was then added with HATU (CAS RN: 148893-10-1; 62 mg, 0.16 mmol) and DIPEA (CAS RN: 7087-68-5; 21 mg, 0.16 mmol) and left stirring for 30 minutes under 0° C. Intermediate L3P2-e (tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-12-oxo-3,6,9-trioxa-13-azaheptadecan-17-oate; 60 mg, 0.054 mmol) was added to the reaction mixture and the reaction mixture was left stirring for additional 1 hour under 0° C. LC-MS was employed to verify the complete conversion of intermediate L3P2-e in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3P2-f as a white solid (tert-butyl (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-3-methyl-6,19-dioxo-10,13,16-trioxa-3,7,20-triazatetracosan-24-oate; 40 mg, 0.03 mmol, 55.56% in yield).

LC-MS (m/z, C62H85FN10O19Si)=1321.6[M+1]⁺& 661.4 [M/2+1]⁺.

### Linker-payload L3P2: (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-3-methyl-6,19-dioxo-10,13,16-trioxa-3,7,20-triazatetracosan-24-oic acid

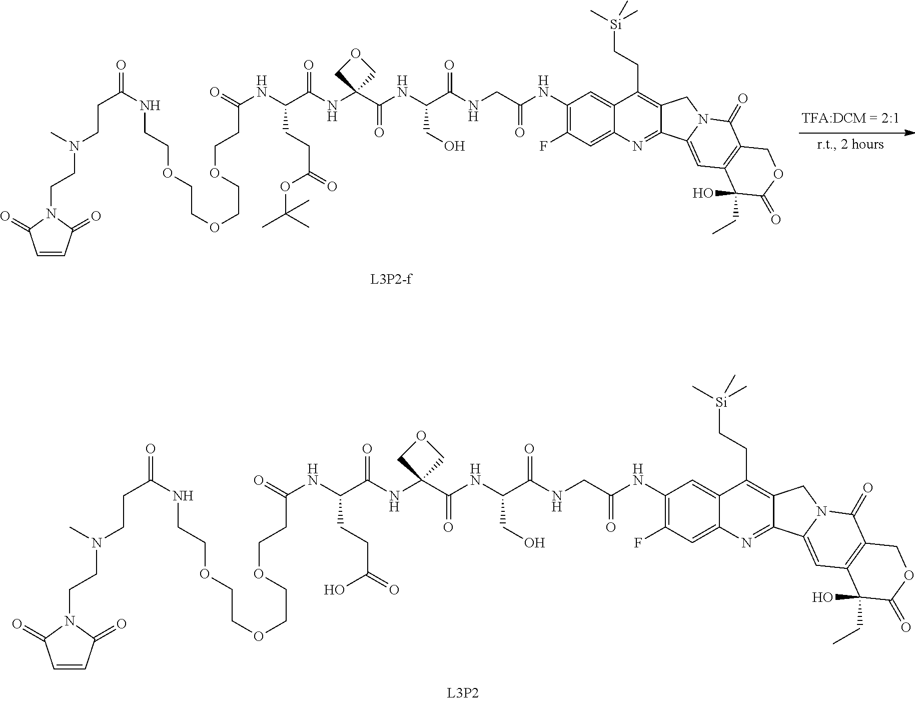

Intermediate L3P2-f (tert-butyl (S)-21-((3-(((S)-6-(dimethylamino)-1-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolazino[1,2-b]quinolin-9-yl)amino)-1-oxyhexan-2-yl)carbamoyl)oxetan-3-yl)carbamoyl)-1-(2,5-dioxo-2,5-dihydro-1H-pyrrol-1-yl)-3-methyl-6,19-dioxo-10,13,16-trioxa-3,7,20-triazatetracosan-24-oate; 40 mg, 0.03 mmol) was dissolved in DCM (CAS RN: 75-09-2, 2 mL) at room temperature. Then, trifluoroacetic acid (CAS RN: 76-05-1; 1 mL, 13.07 mmol) was added to the reaction mixture and stirred for 30 minutes at room temperature. LC-MS was employed to verify the complete consumption of intermediate L3P2-f in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the linker-payload L3P2 as a white solid ((S)-1-(2,5-dioxo-2,5-dihydro-1H-pyrrol-1-yl)-21-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolazino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetane-3-yl)-3-methyl-6,19-dioxo-10,13,16-trioxa-3,7,20-triazatetracosan-24-oic acid; 5.62 mg, 0.004 mmol, 14.80% in yield after purification).

LC-MS (m/z, C58H77FN10O19Si)=1265.4[M+H]⁺& 633.4 [M/2+1]⁺.

¹H NMR (400 MHz, DMSO-d₆) δ 12.19 (s, 1H), 10.06 (s, 1H), 9.27 (s, 1H), 9.16 (s, 1H), 8.96 (d, J=8.4 Hz, 1H), 8.38 (d, J=6.0 Hz, 1H), 8.31 (t, J=6.0 Hz, 1H), 8.24 (s, 1H), 8.04 (d, J=12.0 Hz, 1H), 7.52 (d, J=7.6 Hz, 1H), 7.32 (s, 1H), 7.12 (s, 2H), 6.54 (s, 1H), 5.45 (s, 2H), 5.33 (s, 2H), 4.97 (s, 1H), 4.90 (d, J=6.4 Hz, 1H), 4.77 (d, J=6.4 Hz, 1H), 4.51 (dd, J=24.8, 6.4 Hz, 2H), 4.35 (q, J=5.6 Hz, 1H), 4.12 (q, J=6.4 Hz, 2H), 3.79 (s, 2H), 3.72-3.57 (m, 4H), 3.49 (d, J=8.4 Hz, 6H), 3.42 (t, J=5.6 Hz, 3H), 3.24 (s, 2H), 3.12-3.00 (m, 2H), 2.83 (s, 2H), 2.57 (d, J=11.2 Hz, 2H), 2.47-2.26 (m, 5H), 1.95-1.83 (m, 5H), 1.04-0.81 (m, 5H), 0.02 (s, 9H).

### Example 6: Linker-payload L7P2

### Intermediate L7P2-a: (2R,3S,4S,5R,6R)-2-(acetoxymethyl)-6-((S)-2-amino-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)tetrahydro-2H-pyran-3,4,5-triyl triacetate

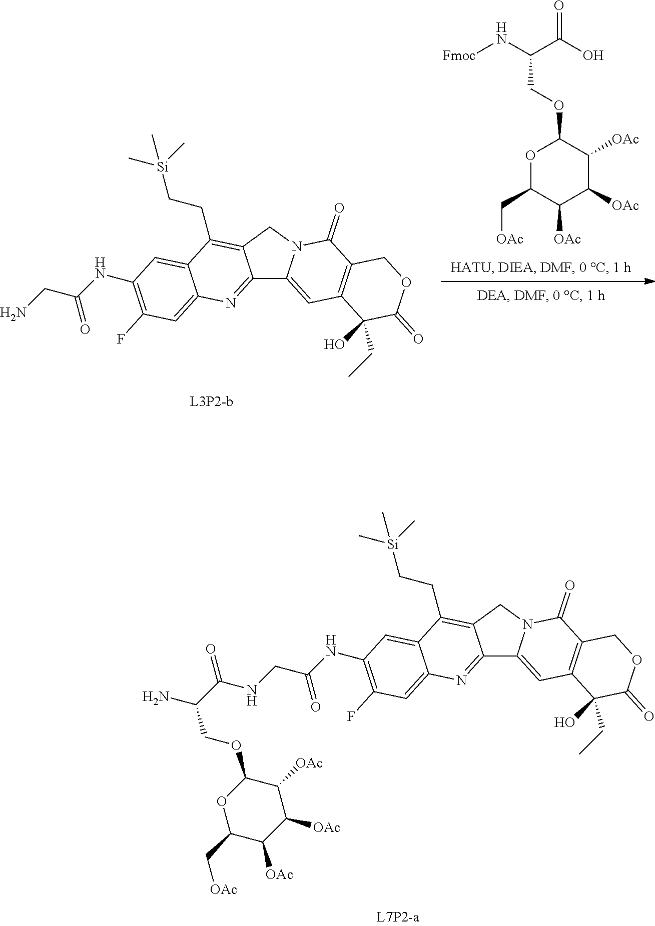

N-(((9H-Fluoren-9-yl)methoxy)carbonyl)-O-((2R,3R,4S,5S,6R)-3,4,5-triacetoxy-6-(acetoxymethyl)tetrahydro-2H-pyran-2-yl)-L-serine (CAS RN: 96383-44-7; 488 mg, 0.74 mmol) was dissolved in 8 ml DMF (CAS RN: 68-12-2) under 0° C. The reaction mixture was then added with HATU (CAS RN: 148893-10-1; 244 mg, 0.64 mmol) and DIPEA (CAS RN: 7087-68-5; 192 mg, 1.49 mmol) and left stirring for 30 minutes under 0° C. Intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 266 mg, 0.44 mmol) was added to the reaction mixture and the reaction mixture was left stirring for additional 1 hour under 0° C. LC-MS was employed to verify the complete conversion of intermediate L3P2-b in the reaction mixture. Then diethylamine (CAS RN: 109-89-7; 400 mg, 5.47 mmol) was added to the reaction mixture and stirred for 1 hour under 0° C. The organic solvent and excess amount of diethylamine in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P2-a as a white solid ((2R,3S,4S,5R,6R)-2-(acetoxymethyl)-6-((S)-2-amino-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)amino)-3-oxopropoxy)tetrahydro-2H-pyran-3,4,5-triyl triacetate; 240 mg, 0.25 mmol, 56.8% in yield).

LC-MS (m/z, C65H93FN10O17Si)=956.4[M+H]⁺.

¹H NMR (400 MHz, DMSO-d6) δ 10.23 (s, 1H), 8.98 (d, J=8.4 Hz, 1H), 8.37 (s, 2H), 8.04 (d, J=12.0 Hz, 1H), 7.32 (s, 1H), 6.54 (s, 1H), 5.45 (s, 2H), 5.33 (s, 2H), 5.28 (d, J=3.6 Hz, 1H), 5.18 (dd, J=10.4, 3.6 Hz, 1H), 4.96 (dd, J=10.4, 8.0 Hz, 1H), 4.76 (d, J=8.0 Hz, 1H), 4.22 (d, J=6.4 Hz, 1H), 4.15-4.03 (m, 4H), 3.83-3.80 (m, 1H), 3.73-3.70 (m, 1H), 3.49 (d, J=5.6 Hz, 2H), 3.08 (s, 2H), 2.13 (s, 3H), 2.04 (d, J=14.8 Hz, 6H), 1.98-1.81 (m, 6H), 0.98-0.85 (m, 5H), 0.18 (s, 9H).

### Intermediate L7P2-b: (2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate

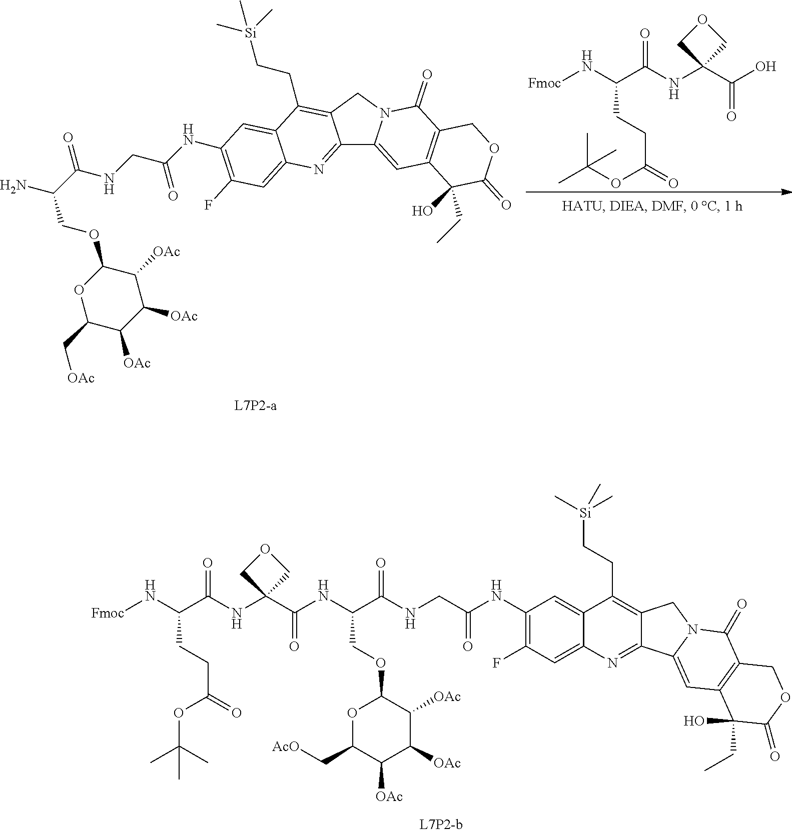

Intermediate L7-b ((S)-3-(2-((((9H-fluoren-9-yl)methoxy)carbonyl)amino)-5-(tert-butoxy)-5-oxopentanamido)oxetane-3-carboxylic acid; 198 mg, 0.38 mmol) was dissolved in 5 ml DMF (CAS RN: 68-12-2) under 0° C. The reaction mixture was then added with HATU (CAS RN: 148893-10-1; 118 mg, 0.31 mmol) and DIPEA (CAS RN: 7087-68-5; 93 mg, 0.72 mmol) and left stirring for 30 minutes under 0° C. Intermediate L7P2-a ((2R,3S,4S,5R,6R)-2-(acetoxymethyl)-6-((S)-2-amino-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)tetrahydro-2H-pyran-3,4,5-triyl triacetate; 240 mg, 0.25 mmol) was added to the reaction mixture and the reaction mixture was left stirring for additional 1 hour under 0° C. LC-MS was employed to verify the complete conversion of intermediate L7P2-a in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P2-b as a white solid ((2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate; 230 mg, 0.16 mmol, 65.94% in yield).

LC-MS (m/z, C72H84FN7O23Si)=1463.4[M+H]⁺.

¹H NMR (400 MHz, DMSO-d₆) δ 10.15 (s, 1H), 9.25 (s, 1H), 8.92 (d, J=8.4 Hz, 1H), 8.47 (s, 1H), 8.28 (s, 1H), 8.01 (d, J=12.0 Hz, 1H), 7.87 (d, J=7.6 Hz, 2H), 7.80-7.69 (m, 4H), 7.39 (t, J=7.5 Hz, 2H), 7.32 (d, J=6.4 Hz, 3H), 6.54 (s, 1H), 5.45 (s, 2H), 5.30 (s, 2H), 5.23 (s, 1H), 5.12 (d, J=10.8 Hz, 1H), 4.95-4.90 (m, 1H), 4.83 (d, J=6.5 Hz, 2H), 4.70 (d, J=8.0 Hz, 1H), 4.59-4.50 (m, 3H), 4.38 (s, 1H), 4.30-4.11 (m, 4H), 4.08-4.01 (m, 5H), 3.85 (d, J=6.6 Hz, 2H), 3.60 (s, 4H), 3.01 (s, 2H), 2.39 (t, J=7.6 Hz, 3H), 2.08 (s, 3H), 1.99 (s, 3H), 1.96 (s, 3H), 1.92-1.85 (m, 6H), 0.91-0.88 (m, 5H), 0.02 (s, 9H).

### Intermediate L7P2-c: tert-butyl (S)-4-amino-5-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano [3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate

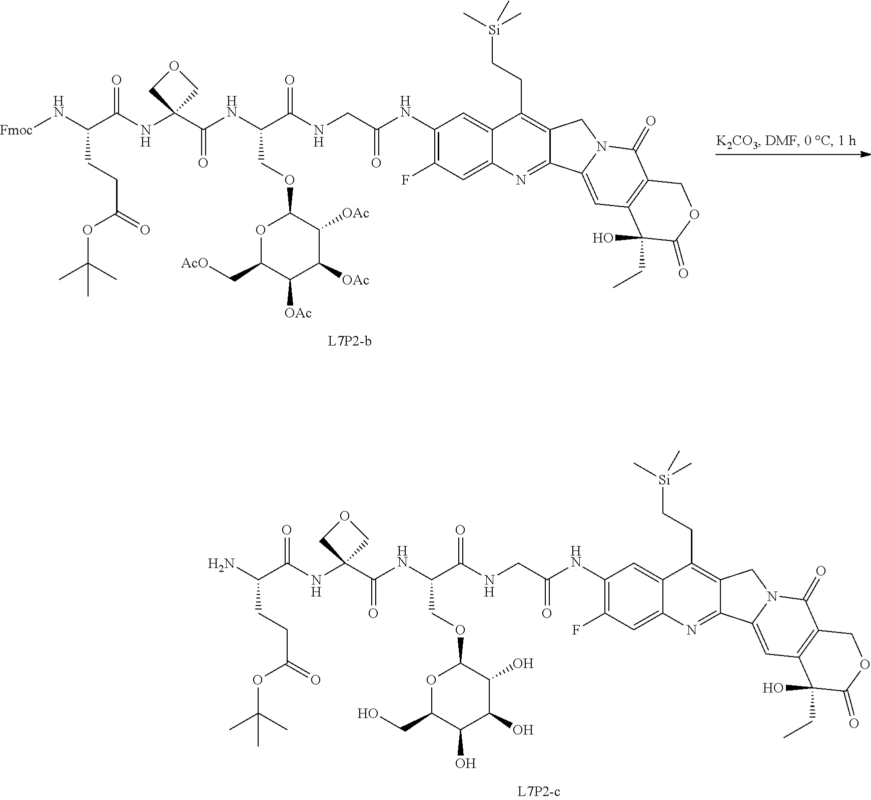

Intermediate L7P2-b ((2R,3R,4S,5S,6R)-2-((S)-2-(3-((S)-2-((((9H-fluoren-9-yl)methoxy)carbonyl)amino)-5-(tert-butoxy)-5-oxopentanamido)oxetane-3-carboxamido)-3-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-oxopropoxy)-6-(acetoxymethyl)tetrahydro-2H-pyran-3,4,5-triyl triacetate, 190 mg, 0.13 mmol) was dissolved in 6 mL DMF (CAS RN: 68-12-2) under 0° C. Then potassium carbonate (CAS RN: 584-08-7; 144 mg, 1.04 mmol) was added to the reaction mixture at 0° C. And the reaction mixture was warmed up to room temperature and stirred for 1 hour. LC-MS was employed to verify the complete conversion of intermediate L7P2-b in the reaction mixture. 0.1 ml formic acid was added to the reaction mixture to neutralize the potassium carbonate. The organic solvent and water in the reaction mixture were reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P2-c as a white solid (tert-butyl (S)-4-amino-5-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano [3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate; 110 mg, 0.10 mmol, 79.00% in yield).

LC-MS (m/z, C49H66FN7O17Si)=1072.4[M+H]⁺.

¹H NMR (400 MHz, DMSO-d₆) δ10.18 (s, 1H), 8.94 (d, J=8.4 Hz, 1H), 8.35 (s, 2H), 8.03 (d, J=12.0 Hz, 1H), 7.92 (d, J=8.0 Hz, 1H), 7.32 (s, 1H), 6.54 (s, 1H), 5.45 (s, 2H), 5.32 (s, 2H), 4.96 (d, J=6.8 Hz, 2H), 4.91-4.79 (m, 3H), 4.75-4.61 (m, 4H), 4.50 (d, J=7.2 Hz, 2H), 4.19-4.11 (m, 4H), 4.06-3.96 (m, 3H), 3.78-3.67 (m, 5H), 3.07 (s, 2H), 2.41 (t, J=8.0 Hz, 2H), 2.04 (s, 2H), 1.92-1.86 (m, 2H), 1.41 (s, 9H), 1.19 (t, J=7.3 Hz, 1H), 0.94-0.87 (m, 5H), 0.18 (s, 9H).

### Intermediate L7P2-d: tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate

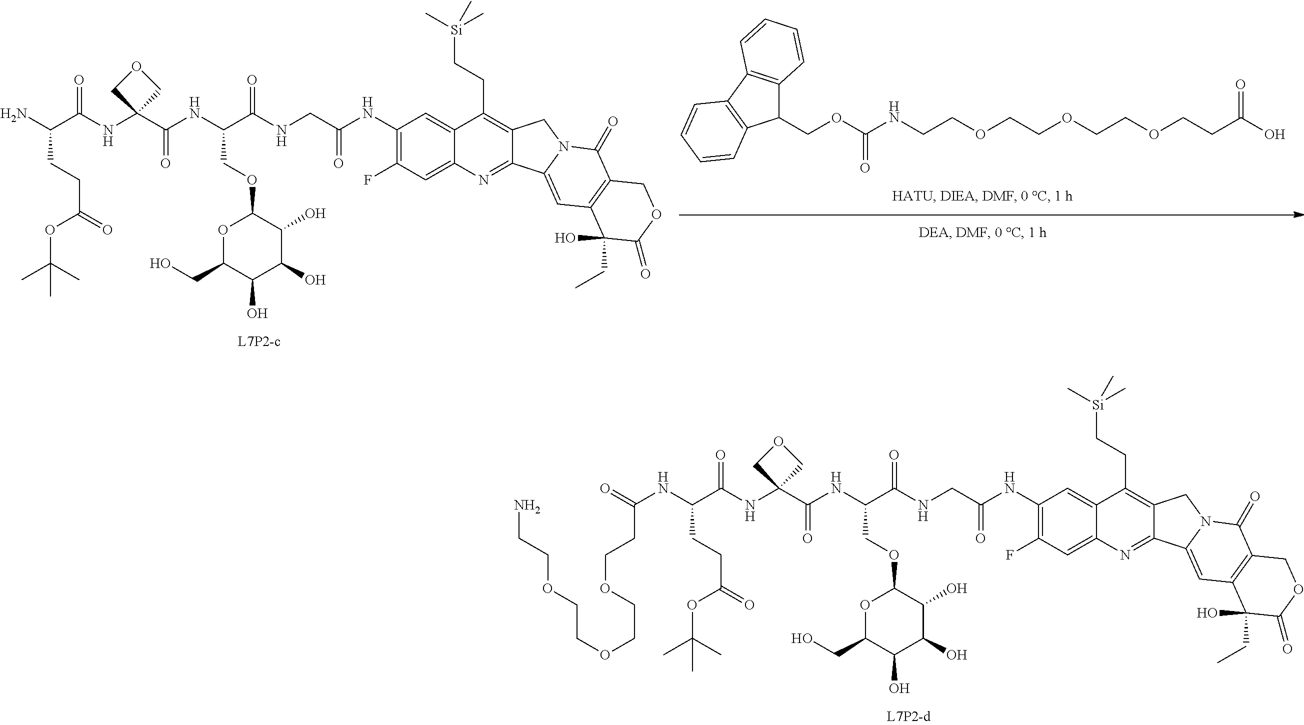

N-Fmoc-12-amino-4,7,10-trioxadodecanoic acid (CAS RN: 867062-95-1; 87 mg, 0.20 mmol) was dissolved in 5 ml DMF (CAS RN: 68-12-2) under 0° C. The reaction mixture was then added with HATU (CAS RN: 148893-10-1; 65 mg, 0.17 mmol) and DIPEA (CAS RN: 7087-68-5; 51 mg, 0.39 mmol) and left stirring for 30 minutes under 0° C. Intermediate L7P2-c (tert-butyl (S)-4-amino-5-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano [3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)amino)-5-oxopentanoate; 140 mg, 0.13 mmol) was added to the reaction mixture. The reaction mixture was left stirring at 0° C. for additional 1 hour. LC-MS was employed to verify the complete conversion of intermediate L7P2-c in the reaction mixture. Then diethylamine (CAS RN: 109-89-7; 146 mg, 2.0 mmol) was added to the reaction mixture and stirred for 1 hour. In the end, 0.2 mL formic acid (CAS RN: 64-18-6) was added to the reaction mixture to neutralize the excess diethylamine. The organic solvent in the reaction mixture was reduced in vacuo to yield the crude product. The crude product was then purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P2-d as a white solid (tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate; 120 mg, 0.0941 mmol, 72.45% in yield).

LC-MS (m/z, C55H81FN8O14Si)=1275.6[M+H]⁺& 638.4[M/2+1]⁺.

1H NMR (400 MHz, DMSO-d6)

### Intermediate L7P2-e: tert-butyl (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate

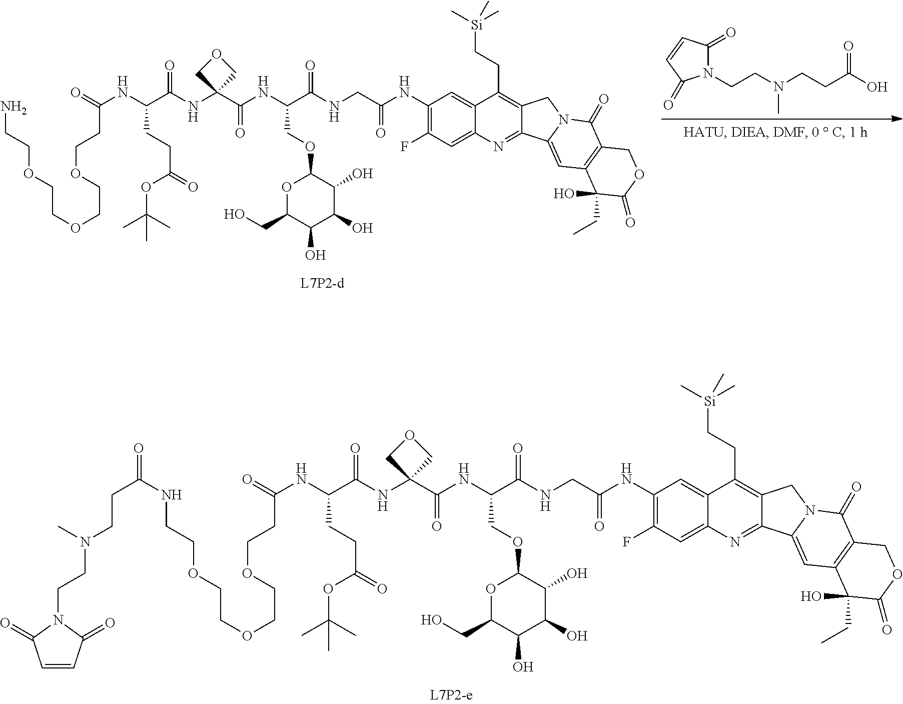

Intermediate L7-c (3-((2-(2,5-dioxo-2,5-dihydro-1H-pyrrol-1-yl)ethyl)(methyl) amino)propanoic acid; 53 mg, 0.23 mmol) was dissolved in 3 ml DMF (CAS RN: 68-12-2) under 0° C. The reaction mixture was then added with HATU (CAS RN: 148893-10-1; 47 mg, 0.12 mmol) and DIPEA (CAS RN: 7087-68-5; 30 mg, 0.23 mmol) and left stirring for 30 minutes under 0° C. Intermediate L7P2-d (tert-butyl (S)-1-amino-14-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-12-oxo-3,6,9-trioxa-13-azaheptadecan-17-oate; 100 mg, 0.078 mmol) was added to the reaction mixture and the reaction mixture was left stirring for additional 1 hour under 0° C. LC-MS was employed to verify the complete conversion of intermediate L7P2-d in the reaction mixture. The organic solvent in the mixture was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the intermediate L7P2-e as a white solid (tert-butyl (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate; 100 mg, 0.067 mmol, 86.44% in yield).

LC-MS (m/z, C68H95FN10O24Si)=1483.6[M+H]⁺& 742.6 [M/2+1]⁺.

¹H NMR (400 MHz, DMSO-d6) δ 10.14 (s, 1H), 9.28 (s, 1H), 9.15 (s, 1H), 8.96 (d, J=8.8 Hz, 1H), 8.23 (s, 2H), 8.03 (d, J=12.1 Hz, 1H), 7.65 (d, J=7.5 Hz, 1H), 7.32 (s, 1H), 7.12 (s, 2H), 6.54 (s, 1H), 5.45 (s, 2H), 5.33 (s, 2H), 4.95-4.73 (m, 4H), 4.66-4.36 (m, 5H), 4.18-4.12 (m, 4H), 3.95 (s, 1H), 3.85-3.41 (m, 27H), 2.83 (s, 3H), 2.29 (d, J=8.0 Hz, 3H), 2.01-1.74 (m, 5H), 1.40 (s, 9H), 1.28-1.20 (m, 1H), 0.91-0.87 (m, 5H), 0.18 (s, 9H).

### Linker-payload L7P2: (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oic acid

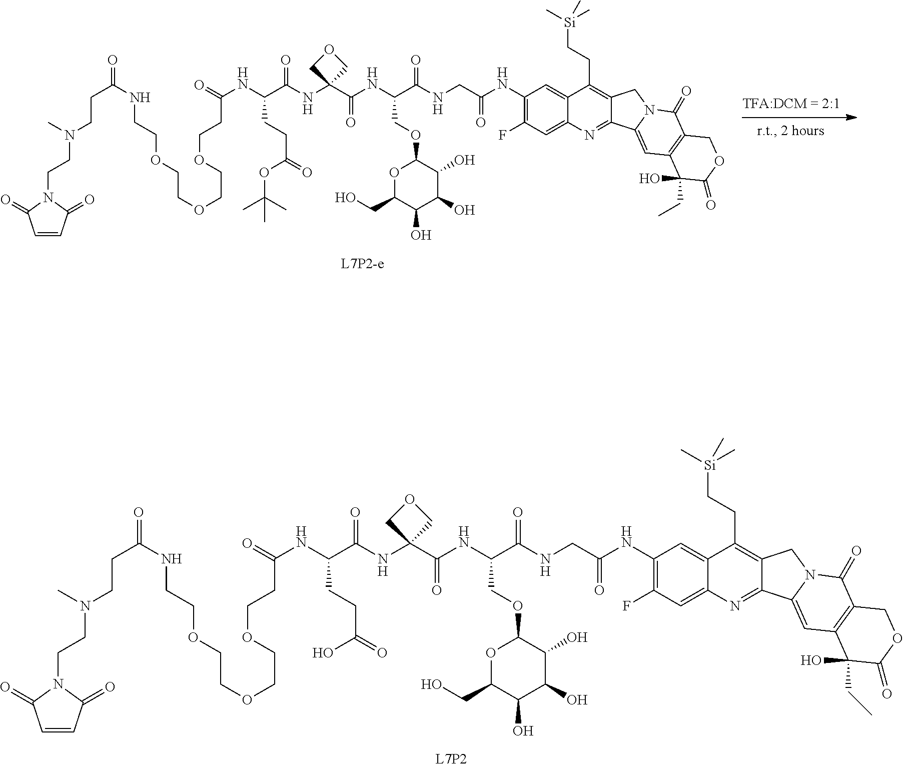

Intermediate L7P2-e (tert-butyl (S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oate; 100 mg, 0.067 mmol) was dissolved in DCM (CAS RN: 75-09-2, 4 mL) at room temperature. Then, trifluoroacetic acid (CAS RN: 76-05-1; 2 mL, 26.14 mmol) was added to the reaction mixture and stirred for 2 hours at room temperature. LC-MS was employed to verify the complete consumption of intermediate L7P2-e in the reaction mixture. The organic solvent and excess TFA acid were reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the linker-payload L7P2 as a white solid ((S)-1-(2,5-dioxo-2,5-dihydro-1H-pyrrol-1-yl)-21-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxo-3-(((2R,3R,4S,5R,6R)-3,4,5-trihydroxy-6-(hydroxymethyl)tetrahydro-2H-pyran-2-yl)oxy)propan-2-yl)carbamoyl)oxetan-3-yl)carbamoyl)-3-methyl-6,19-dioxo-10,13,16-trioxa-3,7,20-triazatetracosan-24-oic acid; 22 mg, 0.015 mmol, 23.03% in yield after purification).

LC-MS (m/z, C64H87FN10O24Si)=713.8[M/2+H].

¹H NMR (400 MHz, DMSO-d₆) δ12.11 (s, 1H), 10.14 (s, 1H), 9.19 (d, J=26.0 Hz, 2H), 8.96 (d, J=8.4 Hz, 1H), 8.24 (s, 2H), 8.04 (d, J=12.0 Hz, 1H), 7.66 (d, J=7.6 Hz, 1H), 7.32 (s, 1H), 7.12 (s, 2H), 6.54 (s, 1H), 5.45 (s, 2H), 5.33 (s, 2H), 5.02-4.72 (m, 4H), 4.64-4.40 (m, 4H), 4.18-4.11 (m, 4H), 3.94 (s, 1H), 3.79 (s, 3H), 3.63 (d, J=19.2 Hz, 4H), 3.53-3.38 (m, 15H), 3.24 (s, 3H), 3.08 (s, 2H), 2.83 (s, 2H), 2.41 (s, 2H), 2.32-2.27 (m, 2H), 1.90-1.83 (m, 5H), 0.91-0.87 (m, 5H), 0.18 (s, 9H).

### Example 7: Linker-Payload L3aP1

### Intermediate L3aP1-a: (S)-2-amino-3-(tert-butoxy)-N-(2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propanamide

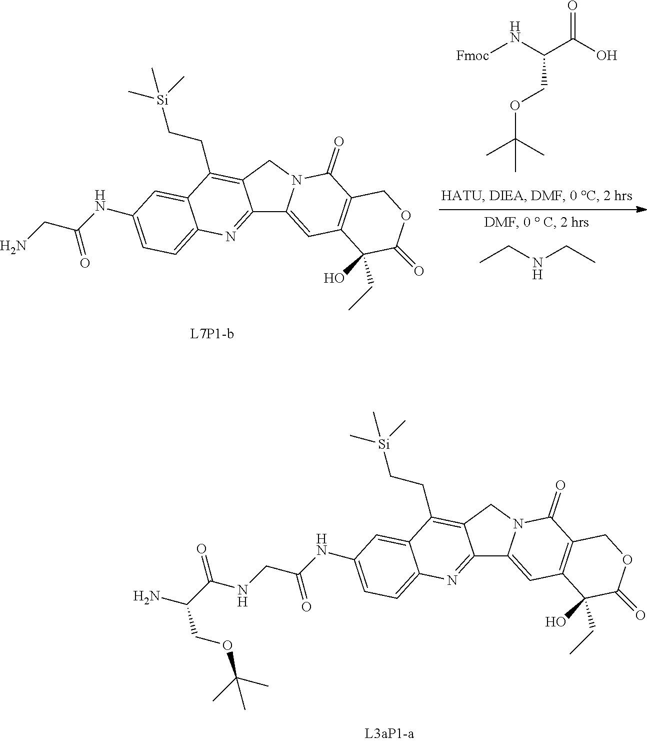

N-(((9H-Fluoren-9-yl)methoxy)carbonyl)-O-(tert-butyl)-L-serine (CAS RN: 71989-33-8; 154.66 mg, 0.40 mmol) was dissolved in 5 ml DMF (CAS RN: 68-12-2) under 0° C. The reaction mixture was added with HATU (CAS RN: 148893-10-1; 153.36 mg, 0.40 mmol) and DIPEA (CAS RN: 7087-68-5; 99.30 mg, 0.77 mmol) and left stirring for 1 hour under 0° C. Intermediate L7P1-b ((S)-2-amino-N-(4-ethyl-4-hydroxy-3,14-dioxo-11-(2-(trimethylsilyl)ethyl)-3,4,12,14-tetrahydro-1H-pyrano [3′,4′:6,7]indolizino[1,2-b]quinolin-9-yl)acetamide, 200 mg, 0.38 mmol) was added to the reaction mixture and the reaction mixture was left stirring for additional 60 minutes under 0° C. LC-MS was employed to verify the complete conversion of intermediate L7P1-b in the reaction mixture. Then the reaction mixture was added with diethylamine (CAS RN: 109-89-7; 0.48 mL, 4.60 mmol) and stirred for 3 hours under 0° C. LC-MS was employed to verify the complete removal of Fmoc protecting group in the mixture. Formic acid solution (CAS RN: 64-18-6; 1 mol/L in water) was added to the reaction mixture to adjust the pH to 2.0-3.0. The excess organic solvent and formic acid in the mixture were reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3aP1-a as a white solid ((S)-2-amino-3-(tert-butoxy)-N-(2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propanamide; 120 mg, 0.18 mmol, 47.11% in yield).

LC-MS (m/z, C34H45N5O7Si)=664.4 [M+H]⁺.

¹H NMR (400 MHz, DMSO) δ 10.52 (s, 1H), 8.70 (m, 1H), 8.38 (m, 2H), 8.13 (m, 1H), 7.87 (m, 1H), 7.30 (s, 1H), 6.50 (s, 2H), 5.45 (s, 2H), 5.30 (s, 2H), 4.05 (s, 2H), 3.53-3.50 (m, 1H), 3.45-3.33 (m, 2H), 3.09-3.00 (m, 2H), 1.93-1.82 (m, 2H), 1.16 (s, 9H), 0.98-0.87 (m, 5H), 0.19 (s, 9H).

### Intermediate L3aP1-b: tert-butyl (S)-4-amino-5-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate

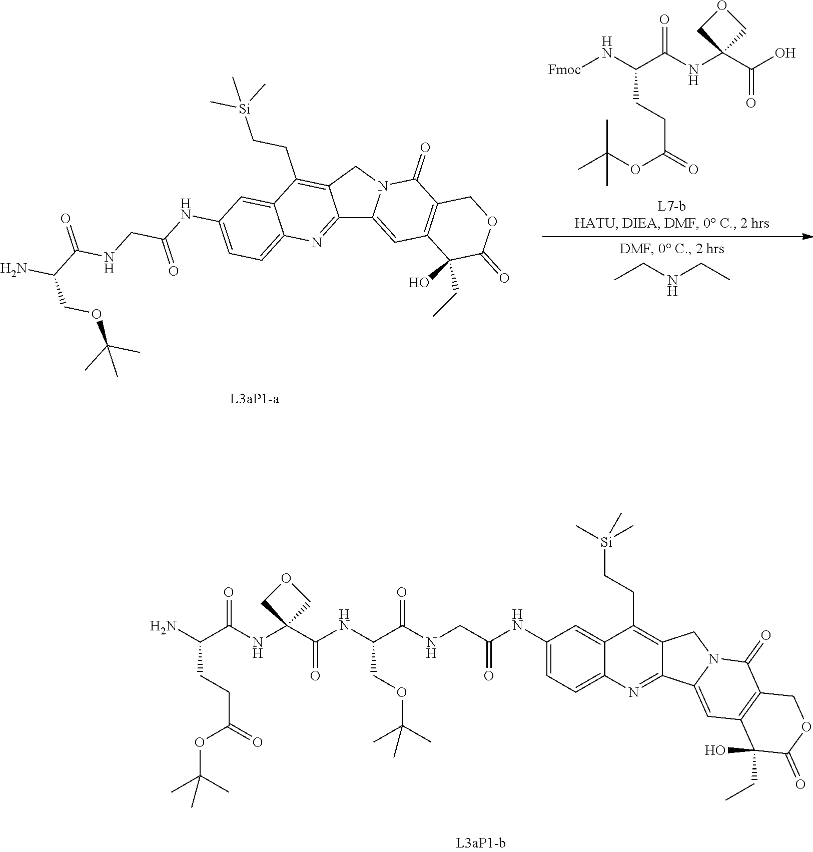

Intermediate L7-b ((S)-3-(2-((((9H-fluoren-9-yl)methoxy)carbonyl)amino)-5-(tert-butoxy)-5-oxopentanamido)oxetane-3-carboxylic acid, 104.31 mg, 0.20 mmol) was dissolved in 8 ml DMF (CAS RN: 68-12-2) under 0° C. The reaction mixture was added with HATU (CAS RN: 148893-10-1; 75.61 mg, 0.20 mmol) and DIPEA (CAS RN: 7087-68-5; 46.73 mg, 0.36 mmol) and left stirring for 1 hour under 0° C. Intermediate L3aP1-a ((S)-2-amino-3-(tert-butoxy)-N-(2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)propanamide, 120 mg, 0.18 mmol) was added to the reaction mixture and the reaction mixture was left stirring for additional 60 minutes under 0° C. LC-MS was employed to verify the complete conversion of intermediate L3aP1-a in the reaction mixture. Then the reaction mixture was added with diethylamine (CAS RN: 109-89-7; 0.24 mL, 2.34 mmol) and stirred for 3 hours under 0° C. LC-MS was employed to verify the complete removal of Fmoc protecting group in the mixture. Formic acid solution (CAS RN: 64-18-6; 1 mol/L in water) was used to adjust the pH value of reaction mixture to 2.0 to 3.0. The excess organic solvent and formic acid in the mixture were reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3aP1-b as a white solid (tert-butyl (S)-4-amino-5-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate; 101 mg, 0.11 mmol, 59.09% in yield).

LC-MS (m/z, C47H65N7O12Si)=948.4[M+H]⁺.

### Intermediate L3aP1-c: tert-butyl (S)-1-amino-17-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)carbamoyl)-15-oxo-3,6,9,12-tetraoxa-16-azaicosan-20-oate

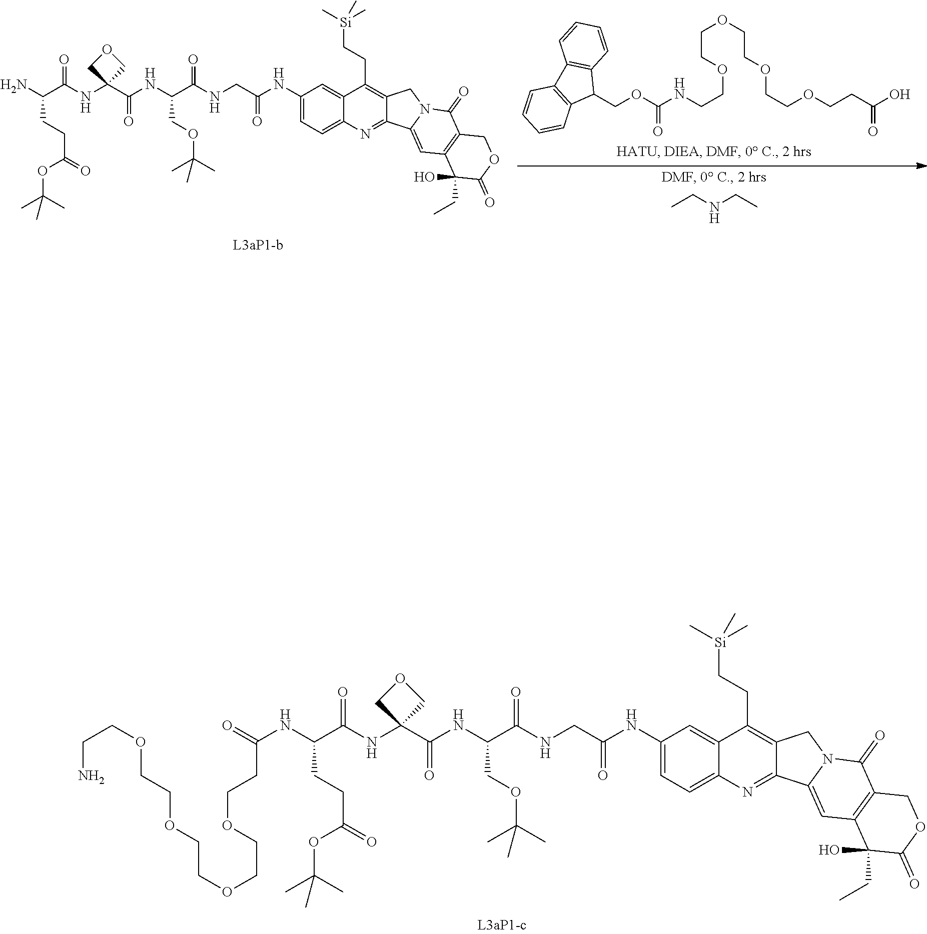

N-Fmoc-12-amino-4,7,10-trioxadodecanoic acid (CAS RN: 867062-95-1, 66.85 mg, 0.14 mmol) was dissolved in 5 ml DMF (CAS RN: 68-12-2) under 0° C. The reaction mixture was added with HATU (CAS RN: 148893-10-1; 48.12 mg, 0.13 mmol) and DIPEA (CAS RN: 7087-68-5; 27.26 mg, 0.21 mmol) and left stirring for 60 minutes under 0° C. Intermediate L3aP1-b (tert-butyl (S)-4-amino-5-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)amino)-5-oxopentanoate, 101 mg, 0.11 mmol) was added to the reaction mixture and the reaction mixture was left stirring for additional 60 minutes under 0° C. LC-MS was employed to verify the complete conversion of intermediate L3aP1-b in the reaction mixture. Then the reaction mixture was added with diethylamine (CAS RN: 109-89-7; 0.13 mL, 1.28 mmol) and stirred for 3 hours under 0° C. LC-MS was employed to verify the complete removal of Fmoc protecting group in the mixture. Formic acid was added to the reaction mixture to adjust to an acidic pH. The excess organic solvent and formic acid in the mixture were reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3aP1-c as a white solid (tert-butyl (S)-1-amino-17-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)carbamoyl)-15-oxo-3,6,9,12-tetraoxa-16-azaicosan-20-oate; 101 mg, 0.08 mmol, 85.55% in yield).

LC-MS (m/z, C58H86N8O17Si)=1197.6[M+H]⁺.

### Intermediate L3aP1-d: (S)-1-amino-17-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-15-oxo-3,6,9,12-tetraoxa-16-azaicosan-20-oic acid

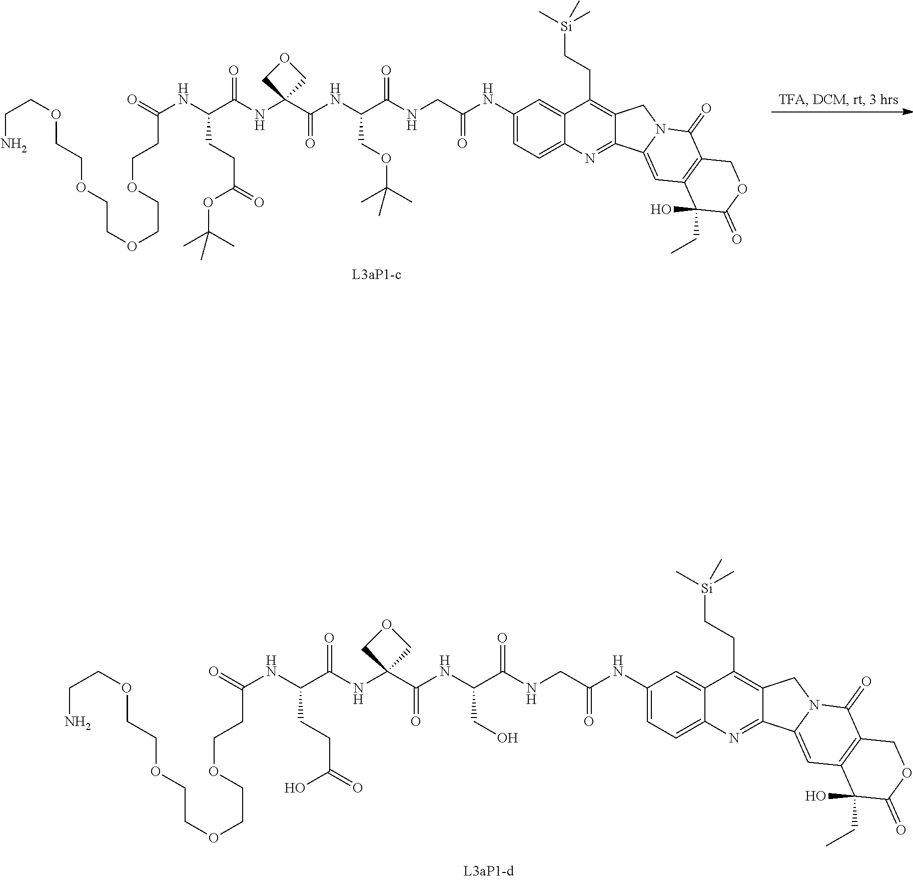

Intermediate L3aP1-c (tert-butyl (S)-1-amino-17-((3-(((S)-3-(tert-butoxy)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)carbamoyl)-15-oxo-3,6,9,12-tetraoxa-16-azaicosan-20-oate; 101 mg, 0.08 mmol) was dissolved in DCM (CAS RN: 75-09-2, 6 mL) at room temperature. Then, trifluoroacetic acid (CAS RN: 76-05-1; 3 mL, 39.20 mmol) was added to the reaction mixture and stirred for 120 minutes at room temperature. LC-MS was employed to verify the complete consumption of intermediate L3aP1-c in the reaction mixture. The organic solvent and excess TFA acid were reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the intermediate L3aP1-d as a white solid ((S)-1-amino-17-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-15-oxo-3,6,9,12-tetraoxa-16-azaicosan-20-oic acid; 50 mg, 0.04 mmol, 47.20% in yield).

LC-MS (m/z, C50H7ON8O17Si)=1084.4[M+H]⁺.

### Linker-payload L3aP1: (S)-4-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoic acid

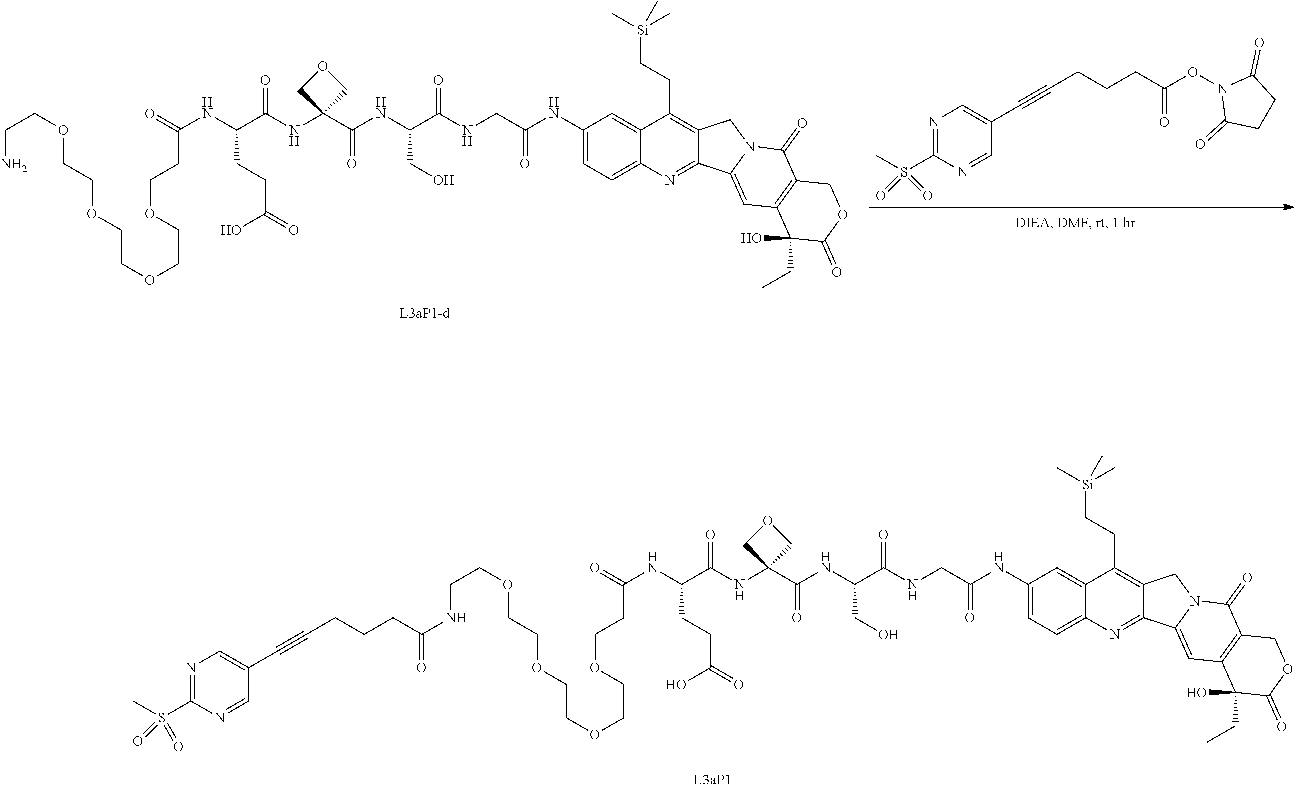

2,5-Dioxopyrrolidin-1-yl 6-(2-(methylsulfonyl)pyrimidin-5-yl)hex-5-ynoate (CAS RN: 2813270-39-0, 18.34 mg, 0.05 mmol) was dissolved in 3 ml DMF (CAS RN: 68-12-2) under 0° C. The reaction mixture was added with intermediate L3aP1-d ((S)-1-amino-17-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-15-oxo-3,6,9,12-tetraoxa-16-azaicosan-20-oic acid; 40 mg, 0.03 mmol) and DIPEA (CAS RN: 7087-68-5; 6.49 mg, 0.05 mmol) and left stirring for 30 minutes under 25° C. LC-MS was employed to verify the complete conversion of intermediate L3aP1-d in the reaction mixture. The excess organic solvent in the mixture was reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the linker-payload L3aP1 as a white solid ((S)-4-((3-(((S)-1-((2-(((S)-4-ethyl-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-3-hydroxy-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoic acid; 16.72 mg, 0.012 mmol, 41.8% in yield).

LC-MS (m/z, C61H80N10O20SSi)=1334.4 [M+H]⁺.

### Example 8: Linker-payload L3aP2

### Intermediate L3aP2-a: N-(3-((S)-5-(tert-butoxy)-2-(22-(2-(methylsulfonyl)pyrimidin-5-yl)-17-oxo-4,7,10,13-tetraoxa-16-azadocos-21-ynamido)-5-oxopentanamido)oxetane-3-carbonyl)-O-(tert-butyl)-L-serine

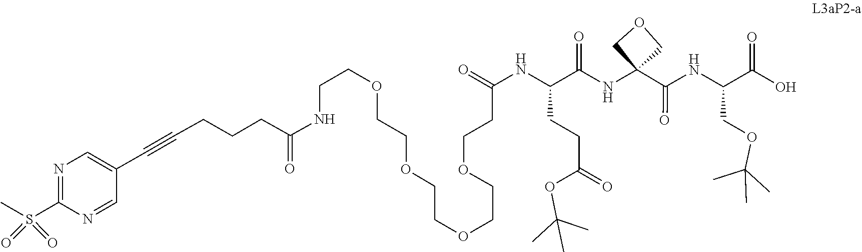

### Synthetic Route for Intermediate L3aP2-a (Solid-Phase Peptide Synthesis)

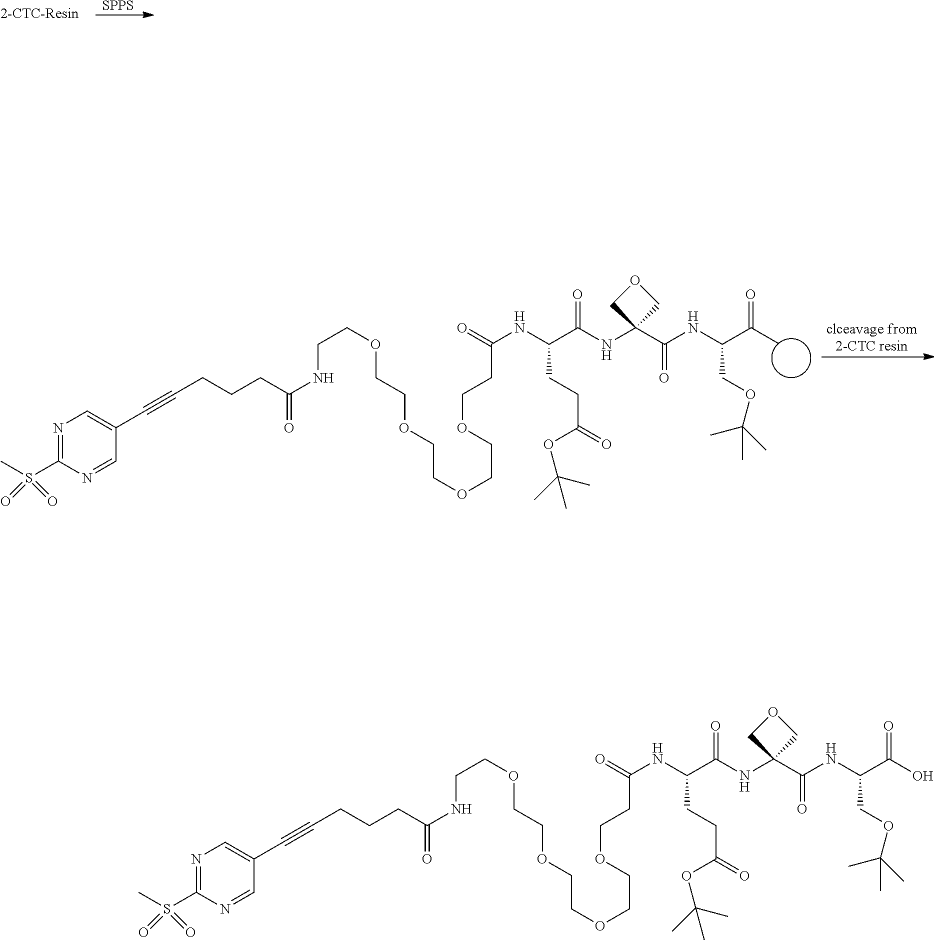

### Synthetic Steps

1.0 g of CTC Resin (CAS RN: 42074-68-0; 1.0 mmol/g. 0.7 mmol) was weighed and added into a 100 ml flask, and DCM (10 ml) was added.

2. DIPEA (CAS RN: 7087-68-5; 361 mg, 2.8 eq) and Fmoc-O-tert-butyl-L-serine (CAS RN: 71989-33-8; 268 mg, 0.7 eq) were weighed and added into the reaction system.

3. The reaction mixture was stirred at 20-25° C. at a rotation speed of 180 rpm for 4 hours.

4. MeOH (1 ml) was measured and added to the reaction system, and the reaction mixture was stirred at 20-25° C. at a rotation speed of 180 rpm for another 1 hour.

5. The reaction mixture was transferred to a 150 ml reactor, and the reaction mixture was removed by suction filtration.

6. The resin was washed with DMF (20 ml×6).

7. 20% piperidine in DMF (20 ml) was added into the reactor, and the reaction was carried out for 30 minutes at room temperature under stirring and nitrogen bubbling.

8. The reaction mixture was removed by suction filtration, the resin was washed with DMF (20 ml×6), a small amount of resin (about 50 beads) was taken out, the resin was tested with ninhydrin chromogenic solution, and both the detection solution and the resin were dark blue.

9. 3-((((9H-Fluoren-9-yl)methoxy)carbonyl)amino)oxetane-3-carboxylic acid (CAS RN: 1380327-56-9; 712 mg, 2.1 mmol), 710 mg of HATU (2.1 mmol) and 361 mg of DIPEA (2.8 mmol) were added to the reactor, the reaction was carried out for 1 hour at room temperature under stirring and nitrogen bubbling.

10. The reaction mixture was removed by suction filtration, the resin was washed with DMF (20 ml×3), a small amount of resin (about 50 beads) was taken out and detected with ninhydrin chromogenic solution, the detection solution was pale yellow, and the resin was colorless and transparent.

11. 20% Pip/DMF (20 ml) was added to the reactor, and the reaction was carried out for 0.5 hours at room temperature with stirring and nitrogen bubbling.

12. The reaction mixture was removed by suction filtration, the resin was washed with DMF (20 ml×6), a small amount of resin (about 50 beads) was taken out, the resin was detected with ninhydrin chromogenic solution, and both the detection solution and the resin were dark blue.

13. 0.892 g of Fmoc-O-tert-butyl-L-glutamic acid (CAS RN: 71989-18-9; 2.1 mmol, 3 eq), 710 mg of HATU (2.1 mmol) and 361 mg of DIPEA were weighed, and the reaction was carried out with stirring and nitrogen bubbling at room temperature for 1 hour.

14. The reaction mixture was removed by suction filtration, the resin was washed with DMF (20 ml×3), a small amount of resin (about 50 beads) was taken out and detected with ninhydrin chromogenic solution, the detection solution was pale yellow, and the resin was colorless and transparent.

15. 20% Pip/DMF (20 ml) was added to the reactor, and the reaction was carried out for 0.5 hours at room temperature under stirring and nitrogen bubbling.

16. The reaction mixture was removed by suction filtration, the resin was washed with DMF (20 ml×6), a small amount of resin (about 50 beads) was taken out, the resin was detected with ninhydrin chromogenic solution, and both the detection solution and the resin were dark blue.

17. 1.02 g of Fmoc-N-amido-PEG4-acid (CAS RN: 557756-85-1; 3 eq, 2.1 mmol), 710 mg of HATU (2.1 mmol) and 361 mg of DIPEA (2.8 mmol) were weighed, and the reaction was carried out under stirring and nitrogen bubbling at room temperature for 1 hour.

18. The reaction mixture was removed by suction filtration, the resin was washed with DMF (20 ml×3), a small amount of resin (about 50 beads) was taken out and detected with ninhydrin chromogenic solution, the detection solution was pale yellow, and the resin was colorless and transparent.

19. 20% Pip/DMF (20 ml) was added to the reactor, and the reaction was carried out for 0.5 hours at room temperature under stirring and nitrogen bubbling.

20. The reaction mixture was removed by suction filtration, the resin was washed with DMF (20 ml×6), a small amount of resin (about 50 beads) was taken out, the resin was detected with ninhydrin chromogenic solution, and both the detection solution and the resin were dark blue.

21. 6-(2-(Methylsulfonyl)pyrimidin-5-yl)hex-5-ynoic acid (CAS RN: 2356229-58-6; 562 mg, 2.1 mmol), 710 mg of HATU (2.1 mmol) and 361 mg of DIPEA (2.8 mmol) were weighed. The reaction was carried out with stirring and nitrogen bubbling at room temperature for 1 hour.

22. The reaction mixture was removed by suction filtration, the resin was washed with DMF (20 ml×3), a small amount of resin (about 50 beads) was taken out and detected with ninhydrin chromogenic solution. The detection solution was pale yellow, and the resin was colorless and transparent.

23. The resin was washed with MeOH (20 ml×3) and methyl tert-butyl ether (20 ml×3), and after being dried for 2 hours, the resin (1.5 g) was obtained.

24. 1.5 g of the resin was weighed and added to a 100 ml eggplant-shaped flask, then 30% HFIP/DCM (15 ml) was added. The reaction mixture was stirred at 20-25° C. at a rotation speed of 180 rpm for 2 hours. The reaction mixture was filtered, the resin was washed with DCM (10 ml×3) and spin-dried, then the product (200 mg) was obtained.

LC-MS (m/z, C₄₂H₆₆N₆O₁₆S)=943.12 [M+H]⁺.

### Intermediate L3aP2-b: tert-butyl (S)-4-((3-(((S)-3-(tert-butoxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoate

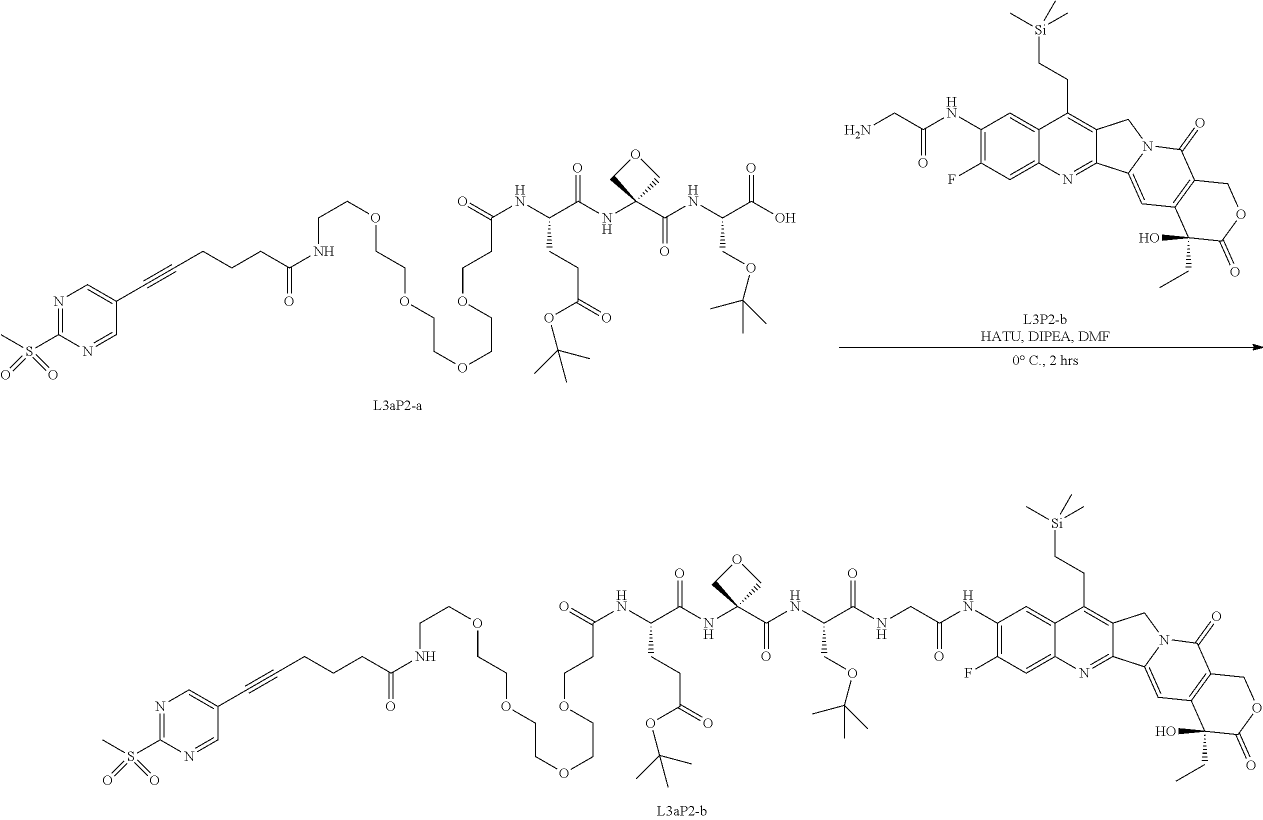

Intermediate L3aP2-a (N-(3-((S)-5-(tert-butoxy)-2-(22-(2-(methylsulfonyl)pyrimidin-5-yl)-17-oxo-4,7,10,13-tetraoxa-16-azadocos-21-ynamido)-5-oxopentanamido)oxetane-3-carbonyl)-O-(tert-butyl)-L-serine; 100 mg, 0.11 mmol) was dissolved in DMF (CAS RN: 68-12-2; 5 ml) at 0° C. Then, HATU (CAS RN: 148893-10-1; 60 mg, 0.16 mmol) and DIPEA (CAS RN: 7087-68-5; 27 mg, 0.21 mmol) were added to the reaction mixture. The reaction mixture was left stirring at 0° C. for 20 minutes before the addition of intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 57 mg, 0.11 mmol). The reaction mixture was left stirring at 0° C. for another 2 hours. The organic solvent in the reaction mixture was reduced in vacuo to afford the reaction residue. The residue was further purified by a preparative reverse-phase chromatography (Column: Welch-Ultimate-C18-10 μm-21.2*150 mm, mobile phase A: 0.1% TFA in H₂O; mobile phase B: ACN) to recover the desired intermediate L3aP2-b as a white solid (tert-butyl (S)-4-((3-(((S)-3-(tert-butoxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)amino)-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoate; 110 mg, 0.075 mmol, 68.27% in yield).

LC-MS (m/z, C₆₉H₉₅FN₁₀O₂₀SSi)=1464.6[M+H]⁺.

### Linker-payload L3aP2: (S)-4-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-3-hydroxy-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoic acid

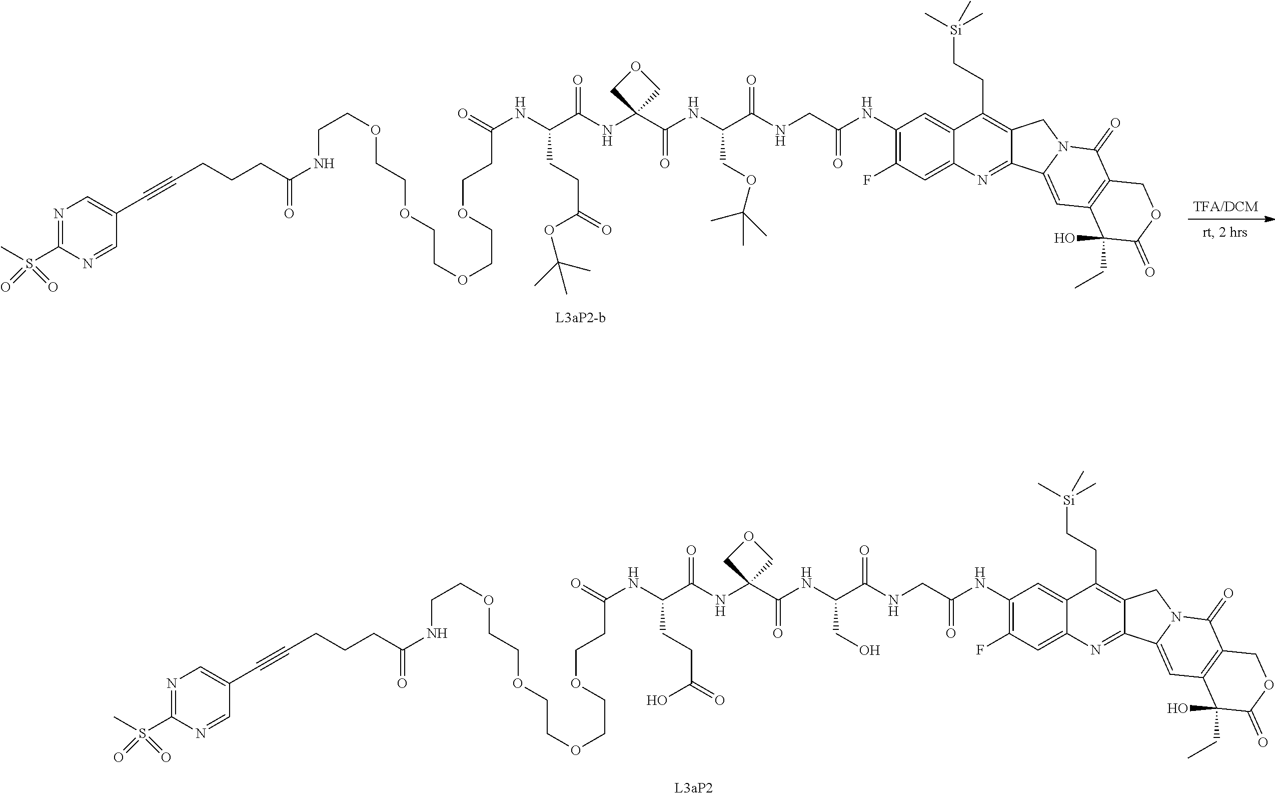

Intermediate L3aP2-b (tert-butyl (S)-4-((3-(((S)-3-(tert-butoxy)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)amino)-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoate; 50 mg, 0.03 mmol) was dissolved in DCM (CAS RN: 75-09-2; 3 ml) at 0° C. TFA (CAS RN: 76-05-1; 1.0 mL) was added to the reaction mixture and the reaction mixture was further stirred at 0° C. for another 2 hours. The organic solvent and excess TFA in the reaction mixture were evaporated in vacuo to afford the reaction residue. The residue was further purified via a preparative reverse-phase liquid chromatography (Column: Welch-Ultimate-C18-10 μm-21.2*150 mm, mobile phase A: 0.1% TFA in H₂O; mobile phase B: ACN) to recover the linker-payload L3aP2 as a white solid ((S)-4-((3-(((S)-1-((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-3-hydroxy-1-oxopropan-2-yl)carbamoyl)oxetan-3-yl)carbamoyl)-27-(2-(methylsulfonyl)pyrimidin-5-yl)-6,22-dioxo-9,12,15,18-tetraoxa-5,21-diazaheptacos-26-ynoic acid; 5 mg, 0.0037 mmol, 12.33% in yield after purification).

LC-MS (m/z, C₆₁H₇₉FN₁₀O₂₀SSi)=1351.2[M+H]⁺.

### Example 9: Linker-Payload L8rP2

### Intermediate L8rP2-a: 6-(2-(methylsulfonyl)pyrimidin-5-yl)-N-(prop-2-yn-1-yl)hex-5-ynamide

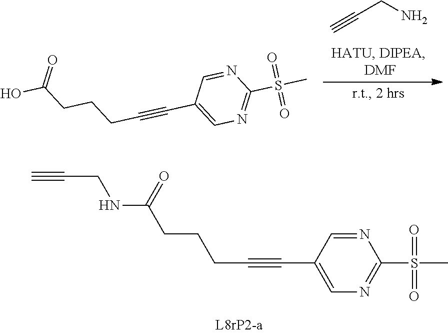

6-(2-Methanesulfonylpyrimidin-5-yl)hex-5-ynoic acid (CAS RN: 2356229-58-6; 400 mg, 1.49 mmol) was dissolved in DMF (CAS RN: 68-12-2; 5 mL) at 0° C. Then HATU (CAS RN: 148893-10-1; 850 mg, 2.24 mmol) and DIPEA (CAS RN: 7087-68-5; 385 mg, 2.98 mmol) were added to the reaction mixture. The reaction mixture was stirred for 30 minutes at 0° C. before the addition of propargylamine (CAS RN: 2450-71-7; 82 mg, 1.49 mmol). The reaction mixture was warmed to room temperature and stirred for another 2 hours. The organic solvent was reduced in vacuo to yield the crude product. The crude product was purified by preparative reverse-phase liquid chromatograph (Column: Welch-Ultimate-C18-10 μm-21.2*150 mm, mobile phase A: 0.1% TFA in H₂O; mobile phase B: ACN) to recover the desired intermediate L8rP2-a (6-(2-(methylsulfonyl)pyrimidin-5-yl)-N-(prop-2-yn-1-yl)hex-5-ynamide; 300 mg, 0.98 mmol, 65.8% in yield).

LC-MS (m/z, C14H15N3O3S)=306.0[M+H]⁺.

¹H NMR (400 MHz, DMSO-d6) δ 9.14 (s, 2H), 8.34 (t, J=5.5 Hz, 1H), 3.88 (dd, J=5.5, 2.5 Hz, 2H), 3.43 (s, 3H), 3.11 (t, J=2.5 Hz, 1H), 2.58 (t, J=7.1 Hz, 2H), 2.30 (t, J=7.4 Hz, 2H), 1.84 (q, J=7.3 Hz, 2H).

### Intermediate L8rP2-b1: ethyl 1-((2-(2-methoxyethoxy)ethyl)carbamoyl)cyclobutane-1-carboxylate

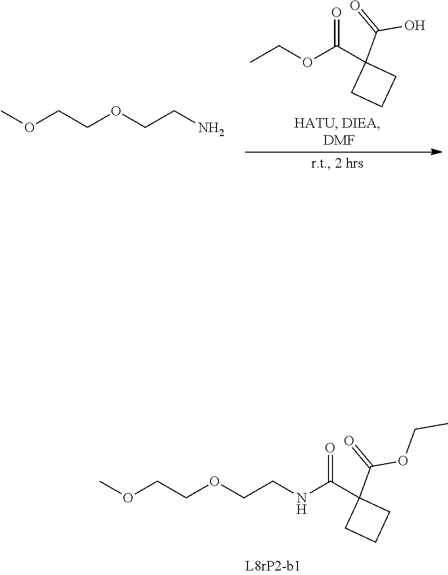

1-(Ethoxycarbonyl)cyclobutane-1-carboxylic acid (CAS RN: 54450-84-9, 1445 mg, 8.39 mmol) was dissolved in DMF (CAS RN: 68-12-2; 20 mL) at 0° C. Then, HATU (CAS RN: 148893-10-1; 4786 mg, 12.59 mmol) and DIPEA (CAS RN: 7087-68-5; 2169 mg, 16.78 mmol) were added to the reaction mixture and the reaction mixture was stirred at 0° C. for 30 minutes. 2-(2-Methoxyethoxy)ethylamine (CAS RN: 31576-51-9; 1000 mg, 8.39 mmol) was added to the reaction mixture at 0° C. and the reaction mixture was stirred for another 2 hours at room temperature. The organic solvent in the reaction mixture was evaporated in vacuo to afford the reaction residue. The residue was further purified by a C18 preparative reverse-phase HPLC (Column: Welch-Ultimate-C18-10 μm-21.2*150 mm, mobile phase A: 0.1% TFA in H₂O; mobile phase B: ACN) to afford the target intermediate L8rP2-b1 (ethyl 1-((2-(2-methoxyethoxy)ethyl)carbamoyl)cyclobutane-1-carboxylate; 1500 mg, 5.49 mmol, 65.4% in yield) as a white solid.

LC-MS (m/z, C13H23NO5)=274.2[M+H]⁺.

### Intermediate L8rP2-b2: 1-((2-(2-methoxyethoxy)ethyl)carbamoyl)cyclobutane-1-carboxylic acid

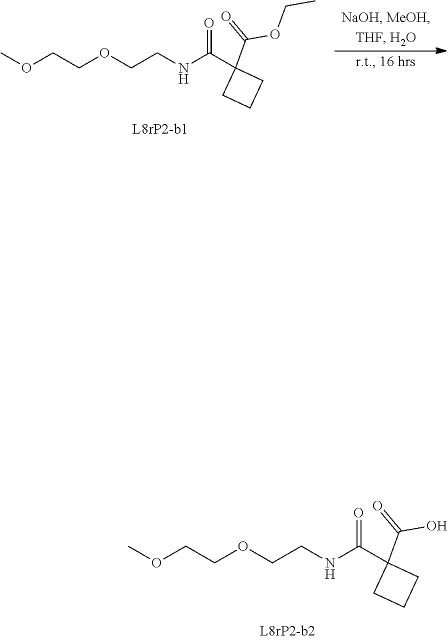

Intermediate L8rP2-b1 (ethyl 1-((2-(2-methoxyethoxy)ethyl)carbamoyl)cyclobutane-1-carboxylate; 1.0 g, 3.66 mmol) was dissolved in a mixture of methanol (CAS RN: 67-56-1; 5 mL), THF (CAS RN: 109-99-9; 5 mL) and water (5 mL). Sodium hydroxide (CAS RN: 1310-73-2; 1.46 g, 36.50 mmol) was added to the reaction mixture at room temperature and stirred for another 16 hours. The organic solvent in the reaction mixture was evaporated in vacuo to afford the reaction residue. The residue was further purified by a preparative reverse-phase HPLC to afford the desired intermediate L8rP2-b2 (1-((2-(2-methoxyethoxy)ethyl)carbamoyl)cyclobutane-1-carboxylic acid; 800 mg, 3.26 mmol, 89.15% in yield) as a white solid.

LC-MS (m/z, C11H19NO5)=246.2[M+H]⁺.

¹H NMR (400 MHz, DMSO-d₆) δ 7.82 (s, 1H), 3.52-3.49 (m, 2H), 3.46-3.37 (m, 4H), 3.25 (s, 3H), 3.24-3.21 (m, 2H), 2.36 (m, 4H), 1.84-1.69 (m, 2H).

### Intermediate L8rP2-c: tert-butyl (2-(hydroxymethyl)-5-nitrobenzyl)carbamate

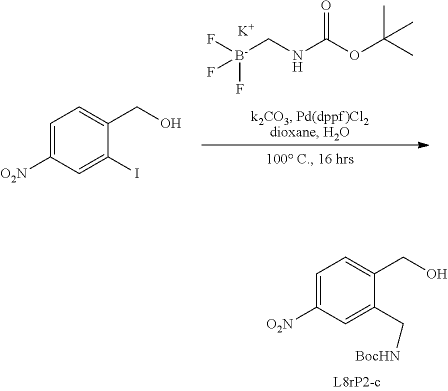 [SMILES: CC(C)(C)OC(=O)NC[B-](F)(F)F ; [K+] ; O=[N+]([O-])c1ccc(CO)c(I)c1 ; "100° C., 16 hrs" ; "k2CO3, Pd(dppf)Cl2dioxane, H2O" ; CC(C)(C)OC(=O)NCc1cc([N+](=O)[O-])ccc1CO]

(2-Iodo-4-nitrophenyl)methanol (CAS RN: 500563-90-6, 500 mg, 1.79 mmol), potassium N-Boc methyltrifluoroborate (CAS RN: 1314538-55-0, 1019 mg, 4.30 mmol), potassium carbonate (CAS RN: 584-08-7, 594 mg, 4.30 mmol) and 1′-bis(diphenylphosphino)ferrocene-palladium(II) dichloride (CAS RN: 72287-26-4, 153 mg, 0.21 mmol) were dissolved in a mixture of water (2 ml) and dioxane (CAS RN: 123-91-1; 10 m1) at room temperature. The reaction mixture was heated to 100° C. and stirred for 16 hours. LC-MS was used to monitor the full consumption of starting material (2-iodo-4-nitrophenyl)methanol in the reaction mixture. The organic solvent in the reaction mixture was reduced in vacuo to afford the reaction residue. The residue was purified by preparative silica chromatography (petroleum ether:ethyl acetate=1:1) to afford the desired intermediate L8rP2-c as a brown oil (tert-butyl (2-(hydroxymethyl)-5-nitrobenzyl)carbamate; 200 mg, 0.71 mmol, 39.58% in yield).

¹H NMR (400 MHz, DMSO-d6) δ 8.15 (dd, J=8.4, 2.5 Hz, 1H), 8.06 (d, J=2.5 Hz, 1H), 7.72 (d, J=8.4 Hz, 1H), 7.55 (t, J=6.0 Hz, 1H), 5.51 (s, 1H), 4.67 (d, J=3.7 Hz, 2H), 4.22 (d, J=6.1 Hz, 2H), 1.43 (s, 9H).

### Intermediate L8rP2-d: tert-butyl (2-(((tert-butyldimethylsilyl)oxy)methyl)-5-nitrobenzyl)carbamate

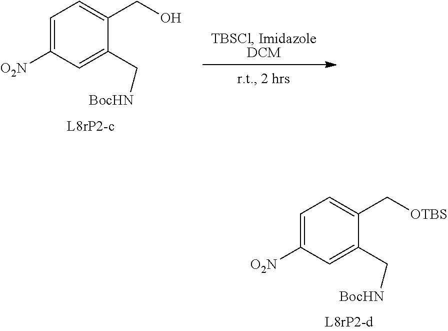 [SMILES: CC(C)(C)OC(=O)NCc1cc([N+](=O)[O-])ccc1CO ; "TBSCl, ImidazoleDCM" ; "r.t., 2 hrs" ; CC(C)(C)OC(=O)NCc1cc([N+](=O)[O-])ccc1CO[Si](C)(C)C(C)(C)C]

Intermediate L8rP2-c (tert-butyl (2-(hydroxymethyl)-5-nitrobenzyl)carbamate; 100 mg, 0.35 mmol) was dissolved in DCM (CAS RN: 75-09-2; 5 mL) at 0° C. Then the reaction mixture was added with imidazole (CAS RN: 288-32-4; 58 mg, 0.85 mmol) and tert-butyldimethylsilyl chloride (CAS RN: 18162-48-6; 96 mg, 0.64 mmol). The reaction mixture was heated up to room temperature and stirred for another 2 hours. LC-MS was employed to confirm the full consumption of starting material in the mixture. The organic solvent in the reaction mixture was evaporated in vacuo to afford the reaction residue. The residue was purified by silica chromatography (petroleum ether:ethyl acetate=5:1) to afford the desired intermediate L8rP2-d as a yellow oil (tert-butyl (2-(((tert-butyldimethylsilyl)oxy)methyl)-5-nitrobenzyl)carbamate; 100 mg, 0.253 mmol, 72.28% in yield).

¹H NMR (400 MHz, DMSO-d₆) δ 8.17 (dd, J=8.4, 2.5 Hz, 1H), 8.08 (d, J=2.4 Hz, 1H), 7.71 (d, J=8.5 Hz, 1H), 7.54 (t, J=6.0 Hz, 1H), 4.88 (s, 2H), 4.23 (d, J=6.1 Hz, 2H), 1.42 (s, 9H), 0.94 (s, 9H), 0.13 (s, 6H).

### Intermediate L8rP2-e: tert-butyl (5-amino-2-(((tert-butyldimethylsilyl)oxy)methyl)benzyl)carbamate

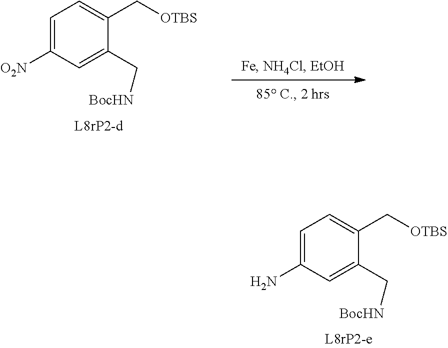

Intermediate L8rP2-d (tert-butyl (2-(((tert-butyldimethylsilyl)oxy)methyl)-5-nitrobenzyl)carbamate, 100 mg, 0.251 mmol) was dissolved in a mixture of ethanol (5 ml) and water (2 ml). Then the reaction mixture was added with ammonium chloride (CAS RN: 12125-02-9, 46 mg, 0.86 mmol) and iron powder (CAS RN: 7439-89-6, 80 mg, 1.43 mmol). The reaction mixture was heated to 85° C. and left stirring for another 2 hours. LC-MS was used to confirm the full conversion of starting material. The organic solvent in the reaction mixture was evaporated in vacuo to afford the reaction residue. The residue was further purified by silica chromatography (petroleum ether:ethyl acetate=1:1) to afford the desired intermediate L8rP2-e as a yellow solid (tert-butyl (5-amino-2-(((tert-butyldimethylsilyl)oxy)methyl)benzyl)carbamate; 50 mg, 0.136 mmol, 54.18% in yield).

¹H NMR (400 MHz, DMSO-d₆) δ 6.99 (d, J=6.0 Hz, 1H), 6.94 (d, J=8.0 Hz, 1H), 6.48 (d, J=2.3 Hz, 1H), 6.39 (dd, J=8.0, 2.4 Hz, 1H), 5.03 (s, 2H), 4.56 (s, 2H), 4.09 (d, J=6.1 Hz, 2H), 1.41 (s, 9H), 0.88 (s, 9H), 0.05 (s, 6H).

### Intermediate L8rP2-f: tert-butyl (S)-(5-(2-((((9H-fluoren-9-yl)methoxy)carbonyl)amino)-6-(dimethylamino)hexanamido)-2-(((tert-butyldimethylsilyl)oxy)methyl)benzyl)carbamate

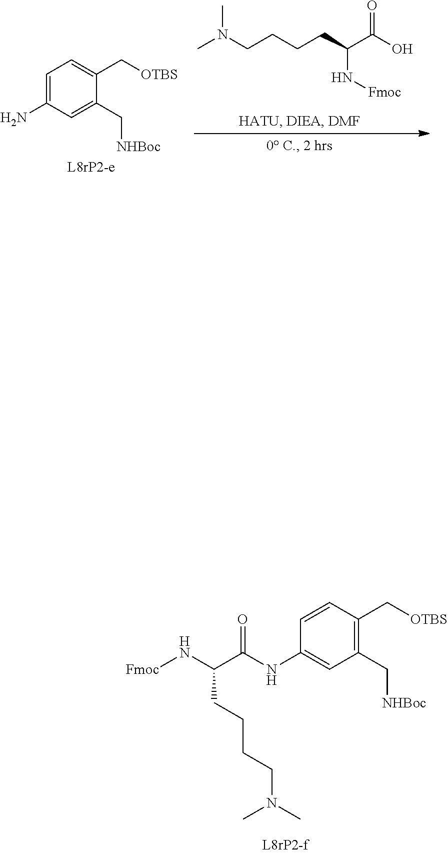

Fmoc-N′,N′-dimethyl-L-lysine hydrochloride (CAS RN: 252049-10-8; 50 mg, 0.13 mmol) was dissolved in DMF (CAS RN: 68-12-2; 2 mL) at 0° C. The reaction mixture was added with HATU (CAS RN: 148893-10-1; 72 mg, 0.19 mmol) and DIPEA (CAS RN: 7087-68-5; 33 mg, 0.26 mmol). The reaction was stirred for 30 minutes at 0° C. before the addition of intermediate L8rP2-e (tert-butyl (5-amino-2-(((tert-butyldimethylsilyl)oxy)methyl)benzyl)carbamate; 46 mg, 0.13 mmol). The reaction mixture was further stirred for 2 hours at 0° C. The organic solvent in the reaction mixture was reduced in vacuo to afford the reaction residue. The residue was further purified via preparative reverse-phase liquid chromatography (Column: Welch-Ultimate-C18-10 μm-21.2*150 mm, mobile phase A: 0.1% TFA in H₂O; mobile phase B: ACN) to afford the desired intermediate L8rP2-f as a white solid (tert-butyl (S)-(5-(2-((((9H-fluoren-9-yl)methoxy)carbonyl)amino)-6-(dimethylamino)hexanamido)-2-(((tert-butyldimethylsilyl)oxy)methyl)benzyl)carbamate; 50 mg, 0.067 mmol, 51.54% in yield).

LC-MS (m/z, C42H60N4O6Si)=745.4[M+H]⁺.

### Intermediate L8rP2-g: tert-butyl (S)-(5-(2-((((9H-fluoren-9-yl)methoxy)carbonyl)amino)-6-(dimethylamino)hexanamido)-2-(hydroxymethyl)benzyl)carbamate

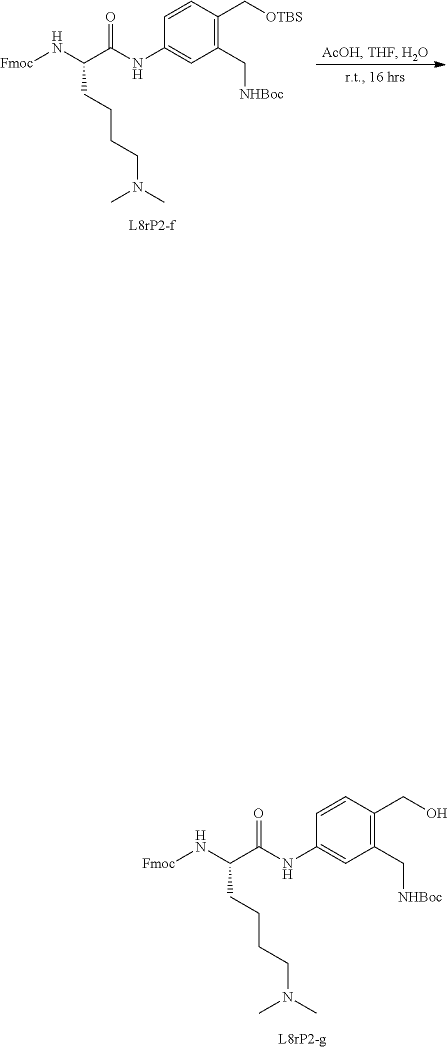

Intermediate L8rP2-f (tert-butyl (S)-(5-(2-((((9H-fluoren-9-yl)methoxy)carbonyl)amino)-6-(dimethylamino)hexanamido)-2-(((tert-butyldimethylsilyl)oxy)methyl)benzyl)carbamate; 1000 mg, 1.34 mmol) was added to a mixture of water (1 ml), acetic acid (CAS RN: 64-19-7; 3 mL) and THF (CAS RN: 109-99-9; 1 mL) at room temperature. The reaction mixture was stirred for 16 hours at room temperature. The organic solvent and excess acetic acid were evaporated in vacuo to afford the reaction residue. The residue was further purified via preparative reverse-phase chromatography (Column: Welch-Ultimate-C18-10 μm-21.2*150 mm, mobile phase A: 0.1% TFA in H₂O; mobile phase B: ACN) to afford the desired intermediate L8rP2-g as a white solid (tert-butyl (S)-(5-(2-((((9H-fluoren-9-yl)methoxy)carbonyl)amino)-6-(dimethylamino)hexanamido)-2-(hydroxymethyl)benzyl)carbamate; 400 mg, 0.635 mmol, 47.39% in yield).

LC-MS (m/z, C36H46N4O6)=631.4[M+H]⁺.

### Intermediate L8rP2-h: tert-butyl (S)-(5-(2-((((9H-fluoren-9-yl)methoxy)carbonyl)amino)-6-(dimethylamino)hexanamido)-2-((((4-nitrophenoxy)carbonyl)oxy)methyl)benzyl)carbamate

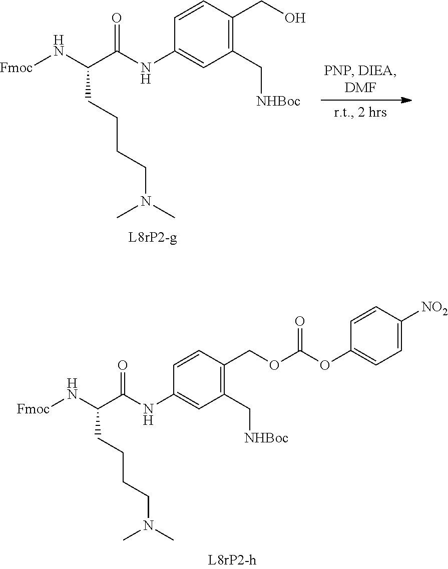

Intermediate L8rP2-g (tert-butyl (S)-(5-(2-((((9H-fluoren-9-yl)methoxy)carbonyl)amino)-6-(dimethylamino)hexanamido)-2-(hydroxymethyl)benzyl)carbamate; 100 mg, 0.16 mmol) was dissolved in DMF (CAS RN: 68-12-2; 2 mL) at room temperature. Then bis(4-nitrophenyl) carbonate (CAS RN: 5070-13-3; 72 mg, 0.24 mmol) and DIPEA (CAS RN: 7087-68-5; 41 mg, 0.32 mmol) were added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rP2-h as a white solid (tert-butyl (S)-(5-(2-((((9H-fluoren-9-yl)methoxy)carbonyl)amino)-6-(dimethylamino)hexanamido)-2-((((4-nitrophenoxy)carbonyl)oxy)methyl)benzyl)carbamate; 100 mg, 0.13 mmol, 78.48% in yield).

LC-MS (m/z, C43H49N5O10)=796.4[M+H]⁺.

### Intermediate L8rP2-i: 4-((S)-2-((((9H-fluoren-9-yl)methoxy)carbonyl)amino)-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

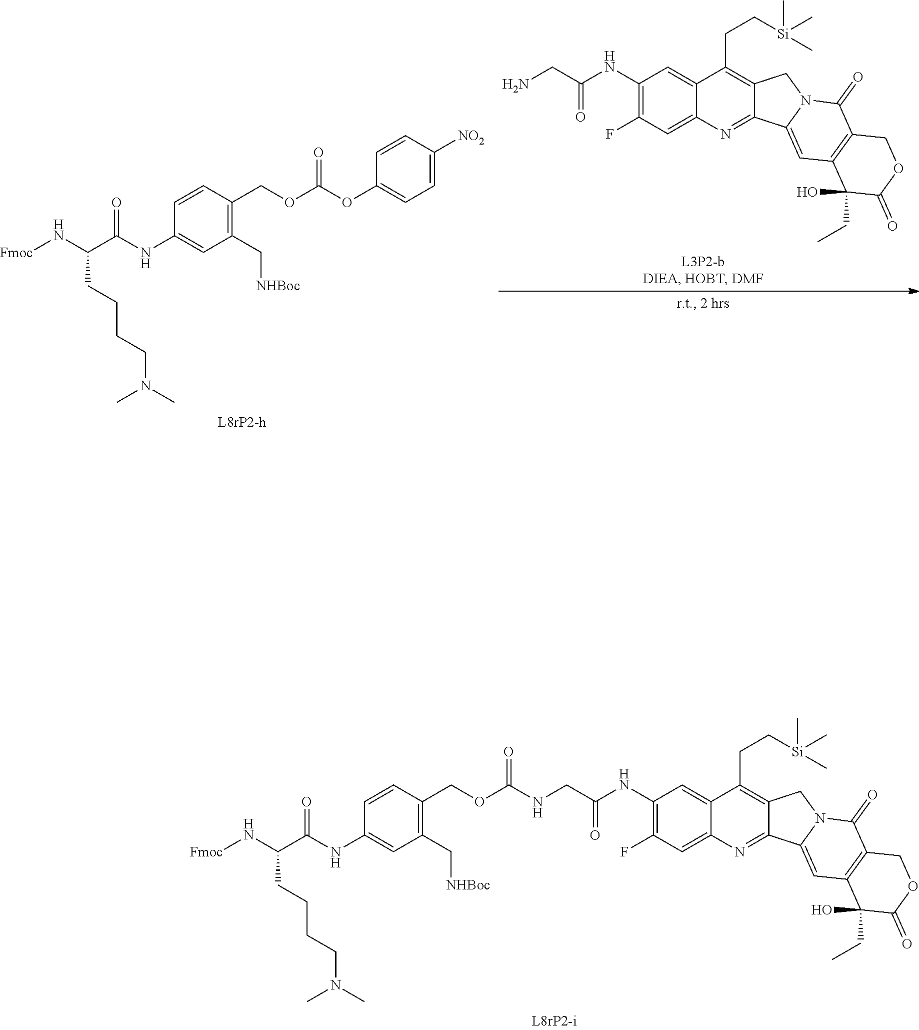

Intermediate L8rP2-h (tert-butyl (S)-(5-(2-((((9H-fluoren-9-yl)methoxy)carbonyl)amino)-6-(dimethylamino)hexanamido)-2-((((4-nitrophenoxy)carbonyl)oxy)methyl)benzyl)carbamate; 50 mg, 0.063 mmol) and intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 34 mg, 0.063 mmol) were dissolved in DMF (CAS RN: 68-12-2; 2 mL) at room temperature. Then HOBt (CAS RN: 2592-95-2; 8.1 mg, 0.06 mmol) and DIPEA (CAS RN: 7087-68-5; 16.24 mg, 0.13 mmol) were added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rP2-i as a white solid (4-((S)-2-((((9H-fluoren-9-yl)methoxy)carbonyl)amino)-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 50 mg, 0.04 mmol, 66.34% in yield).

LC-MS (m/z, C64H75FN8O12Si)=1196.4[M+H]⁺.

### Intermediate L8rP2-j: 4-((S)-2-amino-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamate

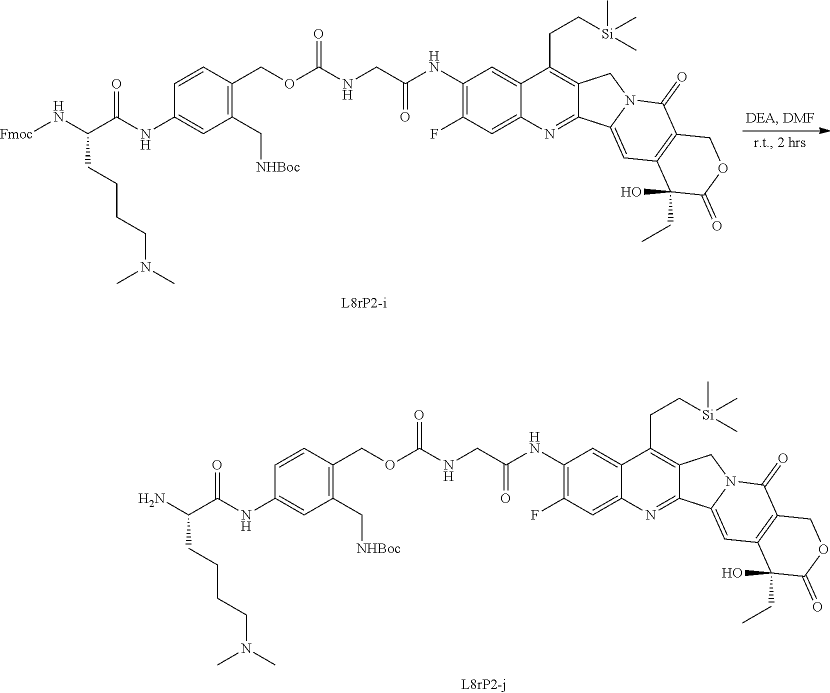

Intermediate L8rP2-i (4-((S)-2-((((9H-fluoren-9-yl)methoxy)carbonyl)amino)-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 20 mg, 0.0167 mmol) was dissolved in DMF (CAS RN: 68-12-2; 2 mL) at room temperature. Then diethylamine (CAS RN: 109-89-7; 12.48 mg, 0.17 mmol) was added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rP2-j as a white solid (4-((S)-2-amino-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 10 mg, 0.01 mmol, 61.51% in yield).

LC-MS (m/z, C49H65FN8O10Si)=973.4[M+H]⁺.

### Intermediate L8rP2-k: 2-(((tert-butoxycarbonyl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

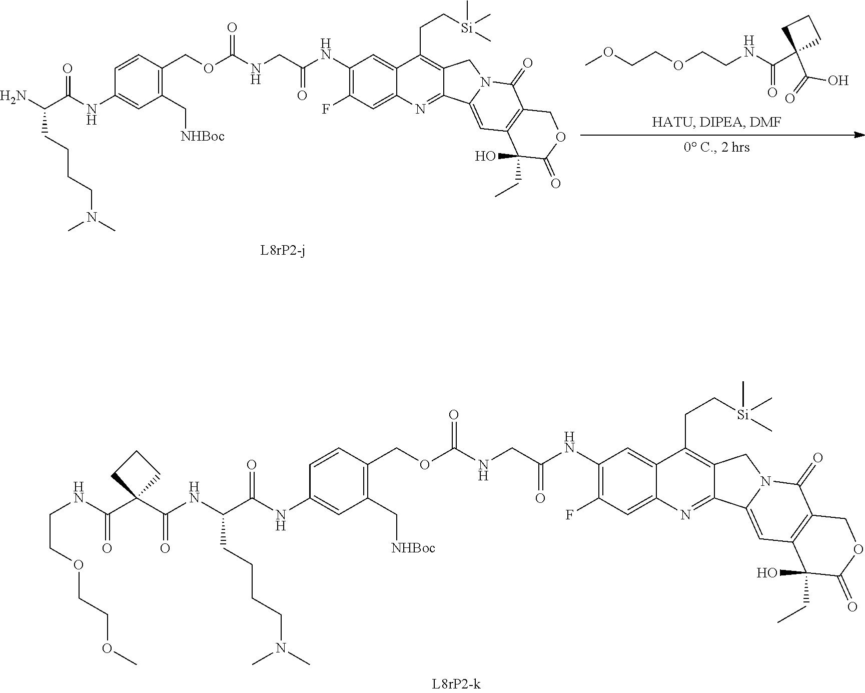

Intermediate L8rP2-b2 (1-((2-(2-methoxyethoxy)ethyl)carbamoyl)cyclobutane-1-carboxylic acid; 5 mg, 0.02 mmol) was dissolved in DMF (CAS RN: 7087-68-5; 1 mL) at 0° C. Then HATU (CAS RN: 148893-10-1; 6 mg, 0.016 mmol) and DIPEA (CAS RN: 7087-68-5; 3 mg, 0.0232 mmol) were added to the reaction mixture. The reaction mixture was stirred at 0° C. for 30 minutes before the addition of intermediate L8rP2-j (4-((S)-2-amino-6-(dimethylamino)hexanamido)-2-(((tert-butoxycarbonyl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 10 mg, 0.01 mmol). The reaction mixture was further stirred at 0° C. for another 2 hours. The organic solvent in the reaction mixture was evaporated in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to afford the desirable intermediate L8rP2-k as a white solid (2-(((tert-butoxycarbonyl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 10 mg, 0.0083 mmol, 81.37% in yield).

LC-MS (m/z, C₆₀H₈₂FN₉O₁₄Si)=1201.6[M+H]⁺.

### Intermediate L8rP2-1: 2-(aminomethyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

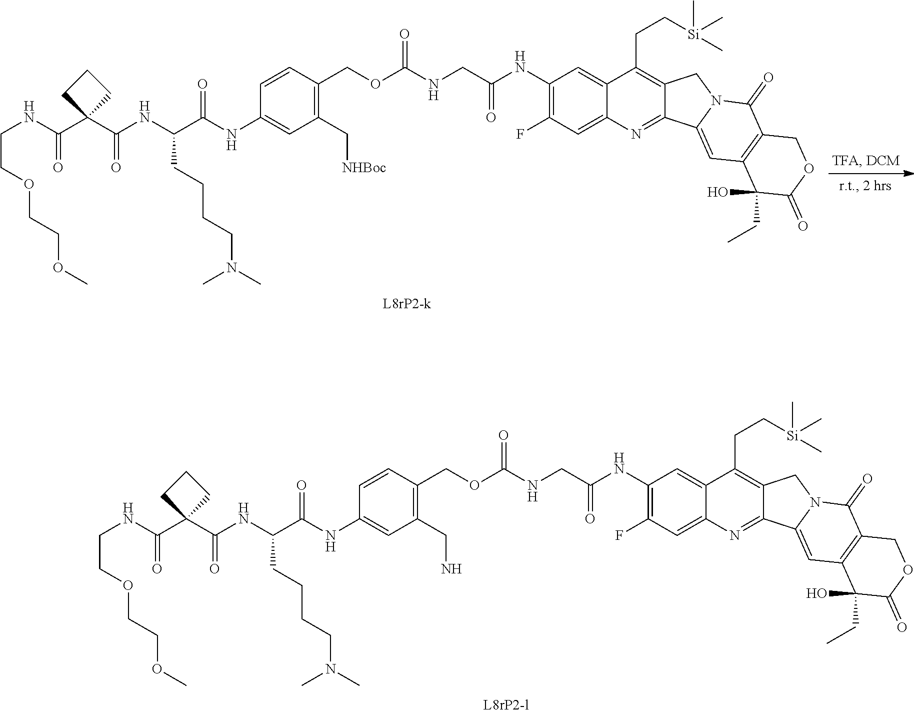

Intermediate L8rP2-k (2-(((tert-butoxycarbonyl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 20 mg, 0.0166 mmol) was dissolved in DCM (CAS RN: 75-09-2; 3 mL) at room temperature. Then TFA (CAS RN: 76-05-1; 1 mL) was added to the reaction mixture and the reaction mixture was stirred for 2 hours at room temperature. The organic solvent was reduced in vacuo to afford the reaction residue. The residue was then purified by preparative reverse-phase liquid chromatography (Column: Welch-Ultimate-C18-10 μm-21.2*150 mm, mobile phase A: 0.1% TFA in H₂O; mobile phase B: ACN) to afford the desirable intermediate L8rP2-1 (2-(aminomethyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 15 mg, 0.0136 mmol, 82.15% in yield).

LC-MS (m/z, C₅₅H₇₄FN₉O₁₂Si)=1100.4[M+H]⁺.

### Intermediate L8rP2-m: 4-((S)-6-(dimethylamino)-2-(1-((2-(2-methoxyethoxy)ethyl)carbamoyl)cyclobutane-1-carboxamido)hexanamido)-2-(((2-methoxy-3,4-dioxocyclobut-1-en-1-yl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

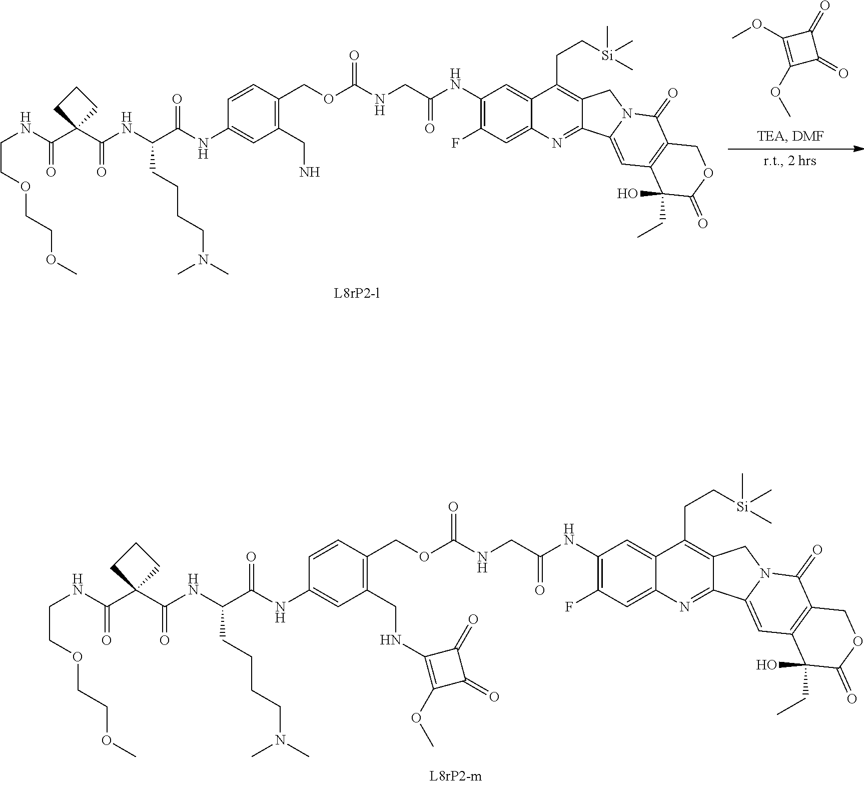

Intermediate L8rP2-1 (2-(aminomethyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 20 mg, 0.0182 mmol) was dissolved in DMF (CAS RN: 68-12-2; 1 mL) at room temperature. Then squaric acid dimethyl ester (CAS RN: 5222-73-1; 2.84 mg, 0.02 mmol) and triethylamine (CAS RN: 121-44-8; 1.84 mg, 0.02 mmol) were added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rP2-m as a white solid (4-((S)-6-(dimethylamino)-2-(1-((2-(2-methoxyethoxy)ethyl)carbamoyl)cyclobutane-1-carboxamido)hexanamido)-2-(((2-methoxy-3,4-dioxocyclobut-1-en-1-yl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 15 mg, 0.13 mmol, 68.18% in yield).

LC-MS (m/z, C₆₀H₇₆FN₉O₁₅Si)=1211.4[M+H]⁺.

### Intermediate L8rP2-n: 2-(((2-((14-azido-3,6,9,12-tetraoxatetradecyl)amino)-3,4-dioxocyclobut-1-en-1-yl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

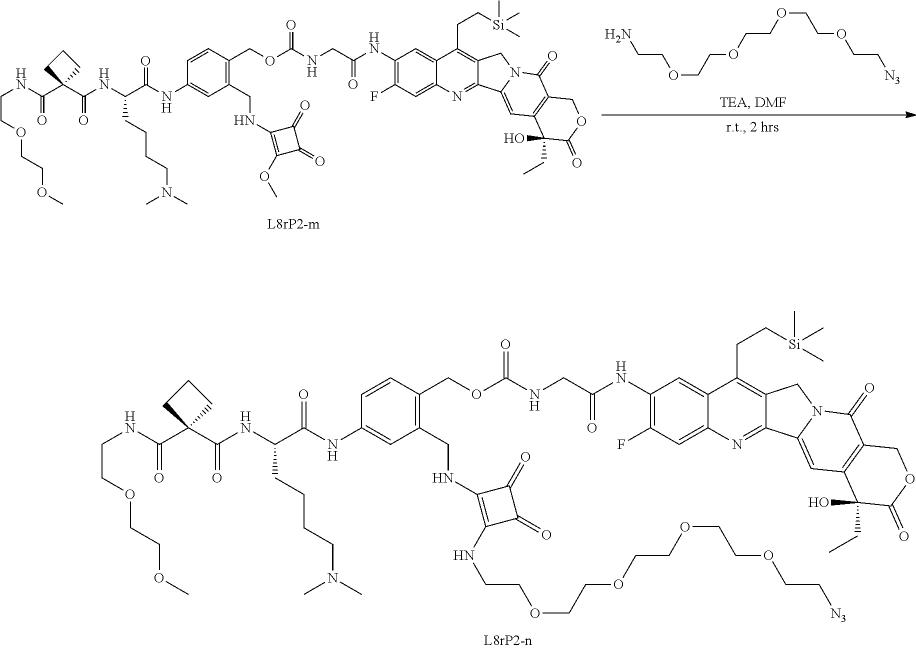

Intermediate L8rP2-m (4-((S)-6-(dimethylamino)-2-(1-((2-(2-methoxyethoxy)ethyl)carbamoyl)cyclobutane-1-carboxamido)hexanamido)-2-(((2-methoxy-3,4-dioxocyclobut-1-en-1-yl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 10 mg, 0.00826 mmol) was dissolved in DMF (CAS RN: 68-12-2; 1 mL) at room temperature. Then 14-amino-3,6,9,12-tetraoxatetradecanyl azide (CAS RN: 951671-92-4; 2.5 mg, 0.01 mmol) and triethylamine (CAS RN: 121-44-8; 1.06 mg, 0.01 mmol) were added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rP2-n as a white solid (2-(((2-((14-azido-3,6,9,12-tetraoxatetradecyl)amino)-3,4-dioxocyclobut-1-en-1-yl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 5 mg, 0.0035 mmol, 42.02% in yield).

LC-MS (m/z, C₆₉H₉₄FN₁₃O₁₈Si)=1441.4[M+H]⁺.

### Linker-payload L8rP2: 4-((S)-6-(dimethylamino)-2-(1-((2-(2-methoxyethoxy)ethyl)carbamoyl)cyclobutane-1-carboxamido)hexanamido)-2-(((2-((14-(4-((6-(2-(methylsulfonyl)pyrimidin-5-yl)hex-5-ynamido)methyl)-1H-1,2,3-triazol-1-yl)-3,6,9,12-tetraoxatetradecyl)amino)-3,4-dioxocyclobut-1-en-1-yl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate

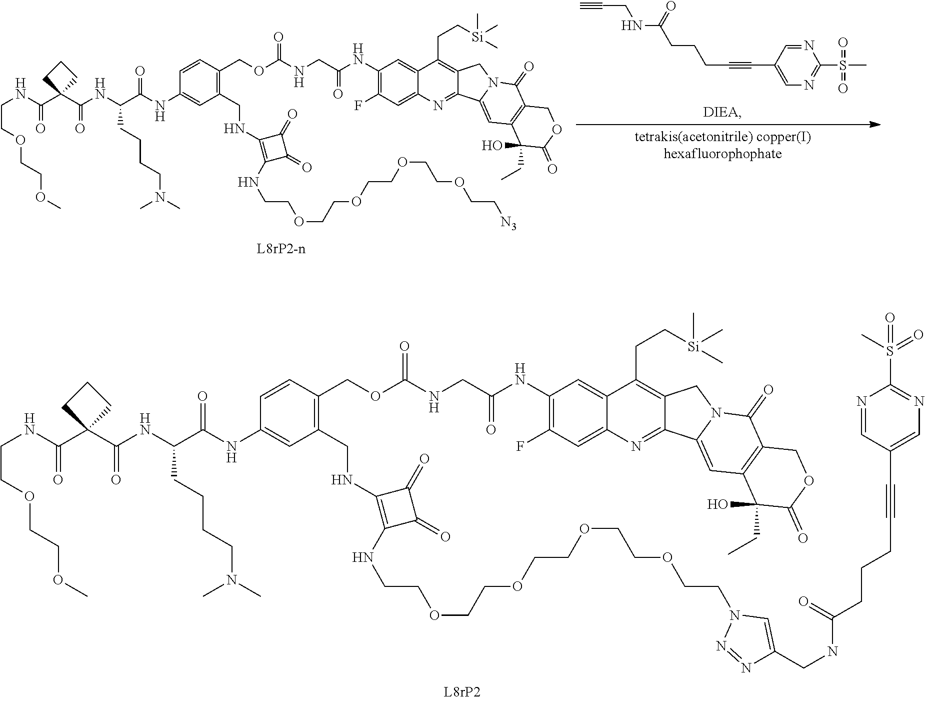

Intermediate L8rP2-n (2-(((2-((14-azido-3,6,9,12-tetraoxatetradecyl)amino)-3,4-dioxocyclobut-1-en-1-yl)amino)methyl)-4-((S)-6-(dimethylamino)-2-(1-((2-(2-methoxyethoxy)ethyl)carbamoyl)cyclobutane-1-carboxamido)hexanamido)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 30 mg, 0.02 mmol) was dissolved in DMF (CAS RN: 68-12-2; 2 mL) at room temperature. Then Intermediate L8rP2-a (6-(2-(methylsulfonyl)pyrimidin-5-yl)-N-(prop-2-yn-1-yl)hex-5-ynamide; 12.7 mg, 0.0415 mmol), tetrakis(acetonitrile) copper(I) hexafluorophosphate (CAS RN: 64443-05-6; 7.7 mg, 0.02 mmol) and DIPEA (CAS RN: 7087-68-5; 5.2 mg, 0.04 mmol) were added to the reaction mixture and the reaction mixture was left stirring for 2 hours at room temperature. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L8rP2 as a white solid (4-((S)-6-(dimethylamino)-2-(1-((2-(2-methoxyethoxy)ethyl)carbamoyl)cyclobutane-1-carboxamido)hexanamido)-2-(((2-((14-(4-((6-(2-(methylsulfonyl)pyrimidin-5-yl)hex-5-ynamido)methyl)-1H-1,2,3-triazol-1-yl)-3,6,9,12-tetraoxatetradecyl)amino)-3,4-dioxocyclobut-1-en-1-yl)amino)methyl)benzyl (2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamate; 4 mg, 0.0023 mmol, 11.45% in yield after purification).

LC-MS (m/z, C83H109FN16O21SSi)=873.8[M/2+H]⁺.

### Example 10: Linker-Payload L18aP2

### Intermediate L18aP2-a: N-(2-(2-(2-azidoethoxy)ethoxy)ethyl)-2-(2,5-dioxo-2,5-dihydro-1H-pyrrol-1-yl)acetamide

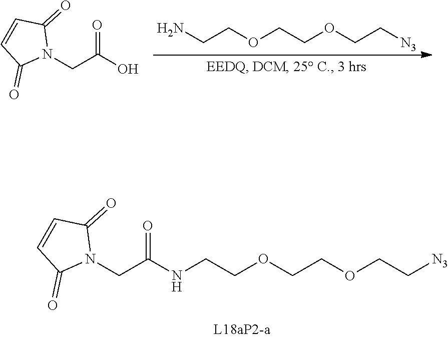 [SMILES: O=C(O)CN1C(=O)C=CC1=O ; [N-]=[N+]=NCCOCCOCCN ; [N-]=[N+]=NCCOCCOCCNC(=O)CN1C(=O)C=CC1=O]

2-(2,5-Dioxo-2,5-dihydro-1H-pyrrol-1-yl)acetic acid (CAS RN: 25021-08-3; 1780 mg, 11.48 mmol) was dissolved in DCM (CAS RN: 75-09-2; 10 ml) under ice-bath. Then, azido-PEG2-amine (CAS RN: 166388-57-4; 1000 mg, 5.74 mmol) and EEDQ (CAS RN: 16357-59-8; 2839 mg, 11.48 mmol) were added to the reaction mixture under ice-bath. The reaction mixture was then warmed to room temperature and stirred for another 3 hours. LC-MS was used to confirm the full consumption of starting material in the reaction mixture. The organic solvent in the reaction mixture was reduced in vacuo to afford the reaction residue. The residue was then purified by silica chromatography (DCM:ACN=10:1) to afford the desirable product L18aP2-a (N-(2-(2-(2-azidoethoxy)ethoxy)ethyl)-2-(2,5-dioxo-2,5-dihydro-1H-pyrrol-1-yl)acetamide) in the eluent (DCM:ACN=10:1) as a colourless liquid. The eluent was used in the next step without further solvent evaporation.

LC-MS (m/z, C12H17N5O5)=312.0 [M+H]⁺.

### Intermediate L18aP2-b: 2-((1-(2-(2-(2-(2-(2,5-dioxo-2,5-dihydro-1H-pyrrol-1-yl)acetamido)ethoxy) ethoxy)ethyl)-1H-1,2,3-triazol-4-yl)methoxy)acetic acid

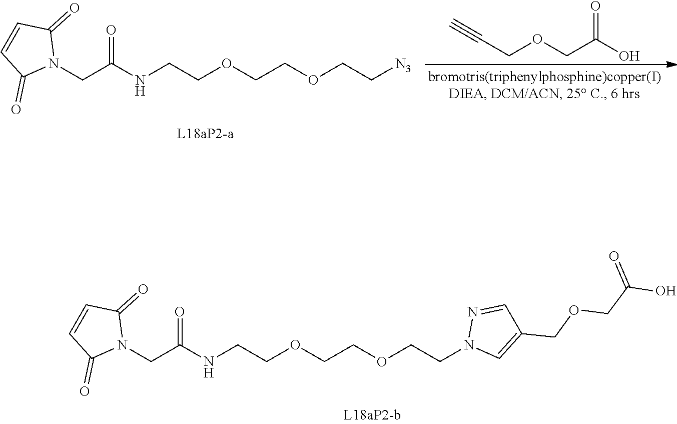

The DCM and acetonitrile mixture containing intermediate L18aP2-a (N-(2-(2-(2-azidoethoxy)ethoxy)ethyl)-2-(2,5-dioxo-2,5-dihydro-1H-pyrrol-1-yl)acetamide) obtained from the last step was added with 2-(prop-2-ynyloxy)acetic acid (CAS RN: 98021-61-5; 350 mg, 3.07 mmol), bromotris(triphenylphosphine) copper(I) (CAS RN: 15709-74-7; 766 mg, 2.06 mmol) and DIPEA (CAS RN: 7087-68-5; 797 mg, 6.17 mmol) at room temperature. The reaction mixture was then stirred at 25° C. for 6 hours. LC-MS was used to confirm the full consumption of starting material in the reaction mixture. The organic solvent in the reaction mixture was reduced in vacuo to afford the reaction residue. The residue was purified by preparative reverse-phase liquid chromatography (acetonitrile/water (formic acid) system) (water (0.1% TFA)/ACN=80%/20%), UV=254 nm, Puninglech-Pntulips-C18-10-30*250 mm, 25° C., 20 mL/min) to afford the desirable intermediate L18aP2-b as a colorless oil (2-((1-(2-(2-(2-(2-(2,5-dioxo-2,5-dihydro-1H-pyrrol-1-yl)acetamido)ethoxy) ethoxy)ethyl)-1H-1,2,3-triazol-4-yl)methoxy)acetic acid, 40 mg, 0.094 mmol, 1.60% in yield over two steps).

LC-MS (m/z, C17H23N5O8)=426.2 [M+H]⁺.

¹H NMR (400 MHz, DMSO-d₆) δ 8.24 (t, J=5.7 Hz, 1H), 8.10 (s, 1H), 7.11 (s, 2H), 4.54 (m, 2H), 4.24 (m, 2H), 3.83 (m, 2H), 3.57-3.54 (m, 4H), 3.50 (m, 2H), 3.39 (m, 2H), 3.20 (m, 2H), 3.08-3.05 (m, 2H).

### Intermediate L18aP2-c: (2S,3R,4S,5S,6S)-2-(2-amino-4-(hydroxymethyl)phenoxy)-6-(methoxycarbonyl)tetrahydro-2H-pyran-3,4,5-triyl triacetate

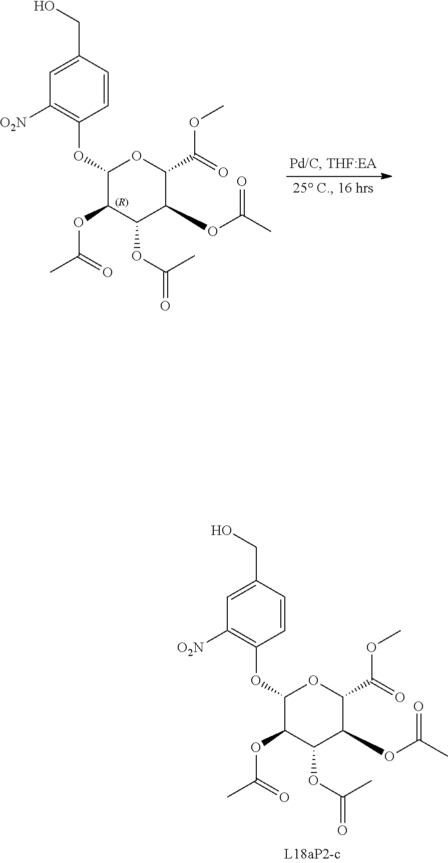

(2S,3R,4S,5S,6S)-2-(4-(Hydroxymethyl)-2-nitrophenoxy)-6-(methoxycarbonyl)tetrahydro-2H-pyran-3,4,5-triyl triacetate (CAS RN: 148579-94-6; 2000 mg, 4.12 mmol) was dissolved in a mixture of THF (CAS RN: 109-99-9; 10 mL) and ethyl acetate (CAS RN: 141-78-6; 10 mL) at room temperature. Then, palladium on carbon (10% wt loading; CAS RN: 7440-05-3; 200 mg, 1.88 mmol) was added to the reaction mixture and the reaction mixture was then stirred under hydrogen atmosphere at room temperature for 16 hours. LC-MS was used to confirm the full consumption of starting material in the reaction mixture. The reaction mixture was filtered to remove palladium on carbon. The organic solvent in the filtrate was reduced in vacuo to obtain the desired intermediate L18aP2-c ((2S,3R,4S,5S,6S)-2-(2-amino-4-(hydroxymethyl)phenoxy)-6-(methoxycarbonyl)tetrahydro-2H-pyran-3,4,5-triyl triacetate; 1800 mg, 3.67 mmol, 89.16% in yield) as a white solid.

LC-MS (m/z, C20H25NO11)=456.2 [M+H]⁺.

¹H NMR (400 MHz, DMSO-d₆) δ 6.83 (d, J=8.2 Hz, 1H), 6.69 (d, J=2.0 Hz, 1H), 6.48 (m, 1H), 5.51 (m, 1H), 5.42 (m, 1H), 5.16-5.05 (m, 2H), 4.99 (m, 1H), 4.69 (m, 1H), 4.61 (s, 2H), 4.34 (m, 2H), 3.67 (s, 3H), 2.06 (s, 3H), 2.02 (m, 6H).

### Intermediate L18aP2-d: (2S,3R,4S,5S,6S)-2-(2-(2-((tert-butoxycarbonyl)amino)acetamido)-4-(hydroxymethyl) phenoxy)-6-(methoxycarbonyl) tetrahydro-2H-pyran-3,4,5-triyl triacetate

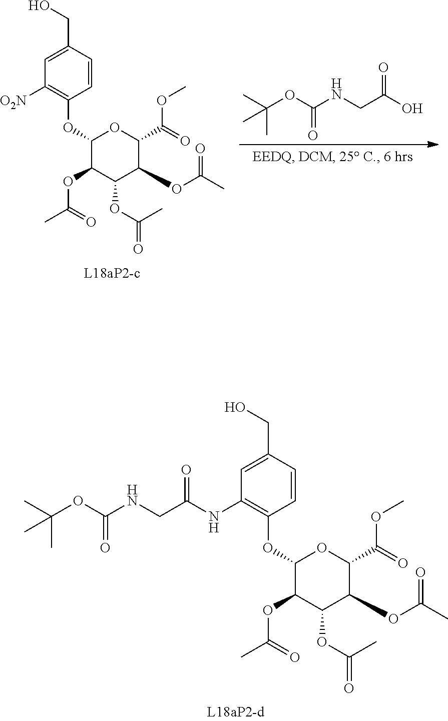 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; CC(C)(C)OC(=O)NCC(=O)O ; COC(=O)[C@H]1O[C@@H](Oc2ccc(CO)cc2NC(=O)CNC(=O)OC(C)(C)C)[C@H](OC(C)=O)[C@@H](OC(C)=O)[C@@H]1OC(C)=O]

Intermediate L18aP2-c ((2S,3R,4S,5S,6S)-2-(2-amino-4-(hydroxymethyl)phenoxy)-6-(methoxycarbonyl)tetrahydro-2H-pyran-3,4,5-triyl triacetate; 455 mg, 1.00 mmol) was dissolved in DCM (CAS RN: 75-09-2; 5 ml) at 0° C. Then EEQD (CAS RN: 16357-59-8; 370 mg, 1.50 mmol) and Boc-glycine (CAS RN: 4530-20-5; 525 mg, 3.00 mmol) were added to the reaction mixture and the reaction mixture was warmed-up to 25° C. The reaction mixture was then stirred for 6 hours at 25° C. LC-MS was used to confirm the full consumption of starting material in the reaction mixture. The organic solvent in the reaction mixture was reduced in vacuo to afford the reaction residue. The residue was purified by preparative reverse-phase liquid chromatography (acetonitrile/water (formic acid) system) (water (0.1% TFA)/ACN=80%/20%), UV=254 nm, Puninglech-Pntulips-C18-10-30*250 mm, 25° C., 20 mL/min) to afford the desirable intermediate L18aP2-d ((2S,3R,4S,5S,6S)-2-(2-(2-((tert-butoxycarbonyl)amino)acetamido)-4-(hydroxymethyl) phenoxy)-6-(methoxycarbonyl) tetrahydro-2H-pyran-3,4,5-triyl triacetate; 600 mg, 0.98 mmol, 98.0% in yield) as a white solid.

LC-MS (m/z, C27H36N2O14)=613.2[M+H]⁺.

¹H NMR (400 MHz, DMSO-d₆) δ 8.76 (s, 1H), 7.96 (s, 1H), 7.06 (m, 3H), 5.60-5.49 (m, 2H), 5.20 (m, 1H), 5.09 (m, 1H), 4.71 (m, 1H), 4.43 (s, 2H), 3.77 (t, J=5.2 Hz, 2H), 3.65 (s, 3H), 3.47 (s, 1H), 2.06 (s, 3H), 2.03 (m, 6H), 1.43 (s, 9H).

### Intermediate L18aP2-e: (2S,3R,4S,5S,6S)-2-(2-(2-((tert-butoxycarbonyl)amino)acetamido)-4-((((4-nitrophenoxy)carbonyl)oxy)methyl)phenoxy)-6-(methoxycarbonyl)tetrahydro-2H-pyran-3,4,5-triyl triacetate

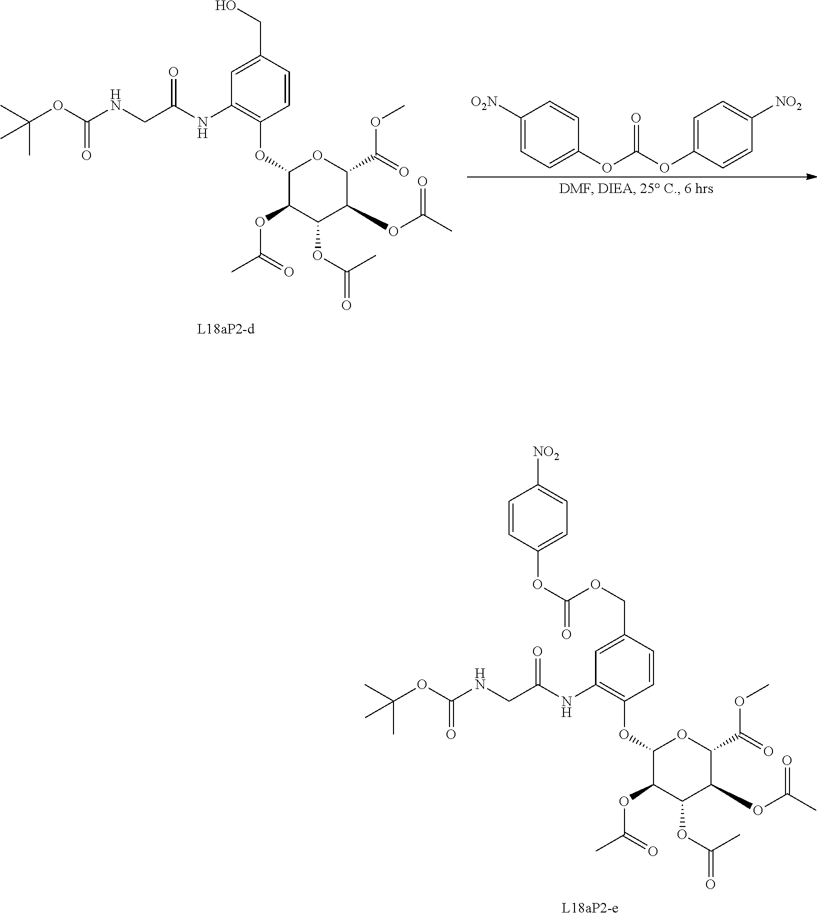 [SMILES: COC(=O)[C@H]1O[C@@H](Oc2ccc(CO)cc2NC(=O)CNC(=O)OC(C)(C)C)[C@H](OC(C)=O)[C@@H](OC(C)=O)[C@@H]1OC(C)=O ; O=C(Oc1ccc([N+](=O)[O-])cc1)Oc1ccc([N+](=O)[O-])cc1 ; COC(=O)[C@H]1O[C@@H](Oc2ccc(COC(=O)Oc3ccc([N+](=O)[O-])cc3)cc2NC(=O)CNC(=O)OC(C)(C)C)[C@H](OC(C)=O)[C@@H](OC(C)=O)[C@@H]1OC(C)=O]

Intermediate L18aP2-d ((2S,3R,4S,5S,6S)-2-(2-(2-((tert-butoxycarbonyl)amino)acetamido)-4-(hydroxymethyl)phenoxy)-6-(methoxycarbonyl) tetrahydro-2H-pyran-3,4,5-triyl triacetate; 800 mg, 1.31 mmol) was dissolved in DMF (CAS RN: 68-12-2; 5 ml) at 0° C. Then bis (4-nitropheny)carbonate (CAS RN: 5070-13-3; 795 mg, 2.62 mmol) and DIPEA (CAS RN: 7087-68-5; 506 mg, 3.91 mmol) were added to the reaction mixture and the reaction mixture was warmed-up to 25° C. The reaction mixture was then stirred for 6 hours at 25° C. LC-MS was used to confirm the full consumption of starting material in the reaction mixture. The organic solvent in the reaction mixture was reduced in vacuo to afford the reaction residue. The residue was purified by preparative reverse-phase liquid chromatography (acetonitrile/water (formic acid) system) (water (0.1% TFA)/ACN=80%/20%), UV=254 nm, Puninglech-Pntulips-C18-10-30*250 mm, 25° C., 20 mL/min) to afford the desirable intermediate L18aP2-e ((2S,3R,4S,5S,6S)-2-(2-(2-((tert-butoxycarbonyl)amino)acetamido)-4-((((4-nitrophenoxy)carbonyl)oxy)methyl)phenoxy)-6-(methoxycarbonyl)tetrahydro-2H-pyran-3,4,5-triyl triacetate; 900 mg, 1.16 mmol, 88.55% in yield) as a white solid.

LC-MS (m/z, C34H39N3O18)=678.2[M-Boc]⁺.

¹H NMR (400 MHz, DMSO-d₆) δ 8.87 (s, 1H), 8.41-8.27 (m, 2H), 8.11 (s, 1H), 7.64-7.54 (m, 2H), 7.25 (dd, J=8.5, 2.1 Hz, 1H), 7.16 (d, J=8.5 Hz, 1H), 7.06 (t, J=6.0 Hz, 1H), 5.67 (m, 1H), 5.53 (m, 1H), 5.27 (s, 2H), 5.22 (m, 1H), 5.09 (m, 1H), 4.75 (m, 1H), 3.85-3.72 (m, 2H), 3.66 (s, 3H), 2.09-1.99 (m, 9H), 1.43 (s, 9H).

### Intermediate L18aP2-f: (2S,3R,4S,5S,6S)-2-(2-(2-((tert-butoxycarbonyl)amino)acetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate

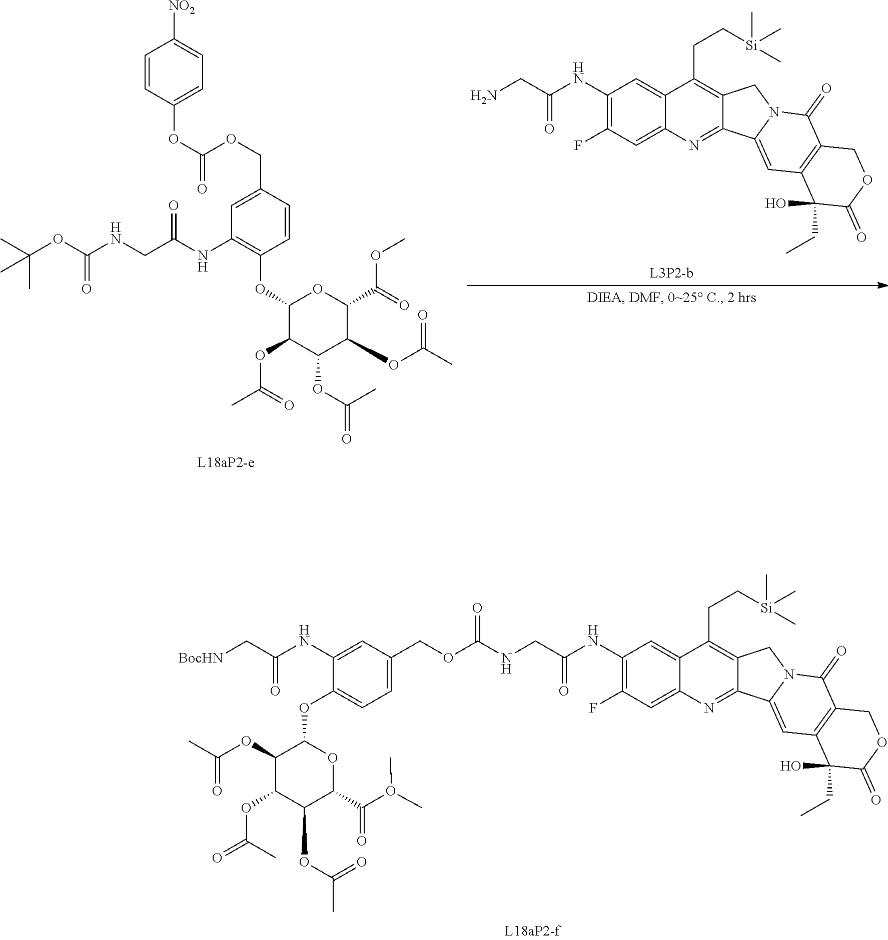

Intermediate L18aP2-e ((2S,3R,4S,5S,6S)-2-(2-(2-((tert-butoxycarbonyl)amino)acetamido)-4-((((4-nitrophenoxy)carbonyl)oxy)methyl)phenoxy)-6-(methoxycarbonyl)tetrahydro-2H-pyran-3,4,5-triyl triacetate; 370 mg, 0.476 mmol) was dissolved in DMF (CAS RN: 68-12-2; 5 mL) at 0° C. Intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 300 mg, 0.556 mmol) and DIPEA (193.6 mg, 1.55 mmol) were added to the reaction mixture at 0° C. The reaction mixture was left stirring for 2 hours at 0° C. LC-MS was used to confirm the full conversion of starting material in the reaction mixture. Formic acid was used to adjust the pH value of reaction mixture to 4.0. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L18aP2-f as a white solid ((2S,3R,4S,5S,6S)-2-(2-(2-((tert-butoxycarbonyl)amino)acetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate; 500 mg, 0.424 mmol, 89.07% in yield).

LC-MS (m/z, C55H65FN6O20Si)=1177.4[M+H]⁺.

### Intermediate L18aP2-g: (2S,3R,4S,5S,6S)-2-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate

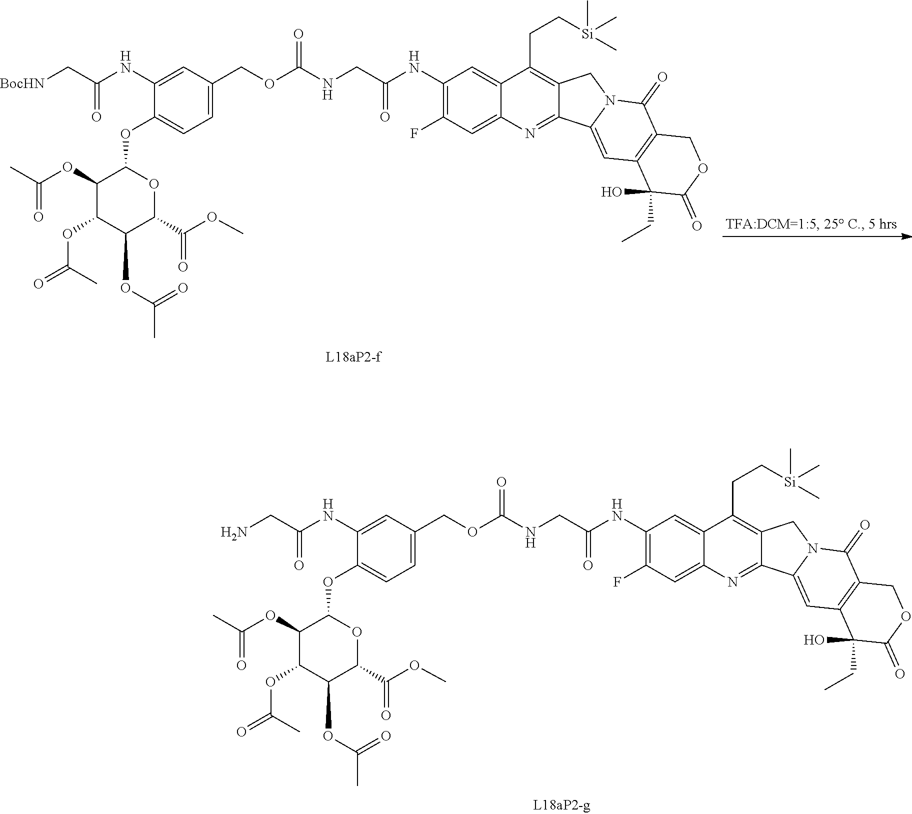 [SMILES: CC[C@@]1(O)C(=O)OCc2c1cc1n(c2=O)Cc2c-1nc1cc(F)c(NC(=O)CNC(=O)OCc3ccc(O[C@@H]4O[C@H](C(=O)OC)[C@@H](OC(C)=O)[C@H](OC(C)=O)[C@H]4OC(C)=O)c(NC(=O)CNC(=O)OC(C)(C)C)c3)cc1c2CC[Si](C)(C)C ; CC[C@@]1(O)C(=O)OCc2c1cc1n(c2=O)Cc2c-1nc1cc(F)c(NC(=O)CNC(=O)OCc3ccc(O[C@@H]4O[C@H](C(=O)OC)[C@@H](OC(C)=O)[C@H](OC(C)=O)[C@H]4OC(C)=O)c(NC(=O)CN)c3)cc1c2CC[Si](C)(C)C]

Intermediate L18aP2-f ((2S,3R,4S,5S,6S)-2-(2-(2-((tert-butoxycarbonyl)amino)acetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate; 500 mg, 0.424 mmol) was dissolved in DCM (CAS RN: 75-09-2, 15 mL) at room temperature. Then, trifluoroacetic acid (CAS RN: 76-05-1; 3 mL, 39.21 mmol) was added to the reaction mixture and stirred for 5 hours at 25° C. LC-MS was employed to verify the complete consumption of intermediate L18aP2-f in the reaction mixture. The organic solvent and excess TFA acid were reduced in vacuo to yield the crude product. The crude product was purified by a preparative reverse-phase liquid chromatograph (Column: Welch-Ultimate-C18-10 μm-21.2*150 mm, mobile phase A: 0.1% TFA in H₂O; mobile phase B: ACN) to recover the desired intermediate L18aP2-g as a yellow solid ((2S,3R,4S,5S,6S)-2-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate; 370 mg, 0.343 mmol, 80.89% in yield).

LC-MS (m/z, C50H57FN6O18Si)=1077.2 [M+H]⁺.

### Intermediate L18aP2-h: (2S,3S,4S,5R,6S)-6-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-3,4,5-trihydroxytetrahydro-2H-pyran-2-carboxylic acid

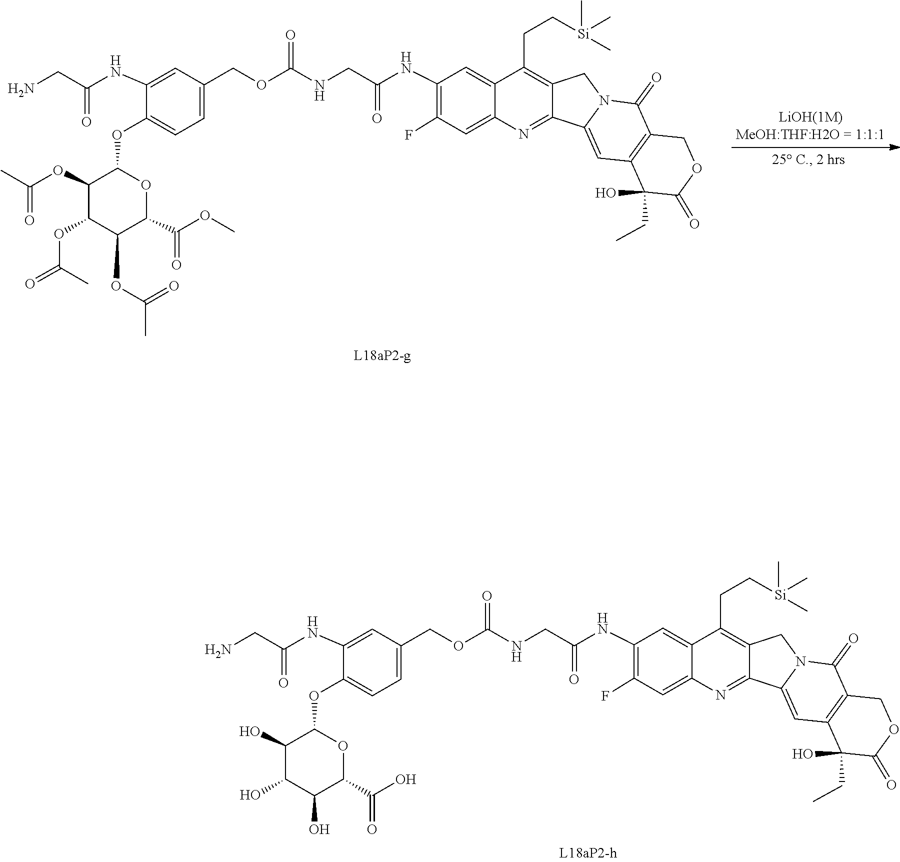 [SMILES: CC[C@@]1(O)C(=O)OCc2c1cc1n(c2=O)Cc2c-1nc1cc(F)c(NC(=O)CNC(=O)OCc3ccc(O[C@@H]4O[C@H](C(=O)OC)[C@@H](OC(C)=O)[C@H](OC(C)=O)[C@H]4OC(C)=O)c(NC(=O)CN)c3)cc1c2CC[Si](C)(C)C ; CC[C@@]1(O)C(=O)OCc2c1cc1n(c2=O)Cc2c-1nc1cc(F)c(NC(=O)CNC(=O)OCc3ccc(O[C@@H]4O[C@H](C(=O)O)[C@@H](O)[C@H](O)[C@H]4O)c(NC(=O)CN)c3)cc1c2CC[Si](C)(C)C]

Intermediate L18aP2-g ((2S,3R,4S,5S,6S)-2-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenoxy)-6-(methoxycarbonyl)tetrahydro-2H-pyran-3,4,5-triyl triacetate; 50 mg, 0.05 mmol) was dissolved in a mixture of water (1 ml), THF (CAS RN: 109-99-9; 1 ml) and methanol (CAS RN: 67-56-1; 1 ml) at 25° C. LiOH aqueous solution (CAS RN: 1310-65-2; 1 mol/L, 0.10 mL, 0.10 mmol) was added to the reaction mixture at 25° C. The reaction mixture was left stirring for 2 hours at 25° C. LC-MS was used to confirm the full conversion of starting material in the reaction mixture. Formic acid was used to adjust the pH value of reaction mixture to 4.0. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L18aP2-h as a yellow solid ((2S,3S,4S,5R,6S)-6-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-3,4,5-trihydroxytetrahydro-2H-pyran-2-carboxylic acid; 36 mg, 0.038 mmol, 76.80% in yield).

LC-MS (m/z, C43H49FN6O15Si)=937.2 [M+H]⁺.

### Linker-payload L18aP2: (2S,3S,4S,5R,6S)-6-(2-(2-(2-((1-(2-(2-(2-(2-(2,5-dioxo-2,5-dihydro-1H-pyrrol-1-yl)acetamido)ethoxy)ethoxy)ethyl)-1H-1,2,3-triazol-4-yl)methoxy)acetamido)acetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-3,4,5-trihydroxytetrahydro-2H-pyran-2-carboxylic acid

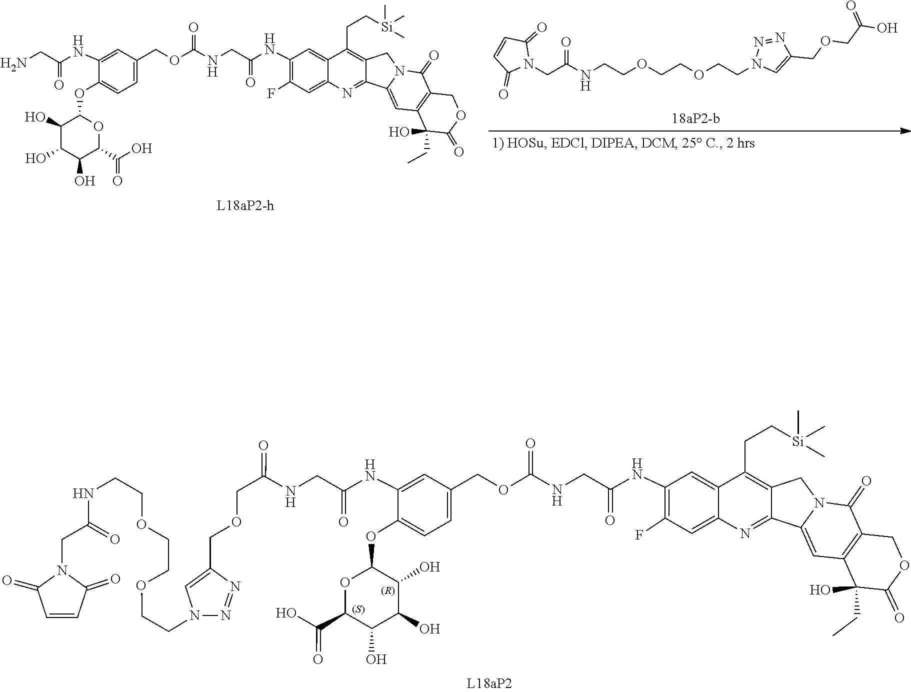

Intermediate L18aP2-b (2-((1-(2-(2-(2-(2-(2,5-dioxo-2,5-dihydro-1H-pyrrol-1-yl)acetamido)ethoxy) ethoxy)ethyl)-1H-1,2,3-triazol-4-yl)methoxy)acetic acid; 40 mg, 0.094 mmol) was dissolved in DCM (CAS RN: 75-09-2; 5 ml) at 25° C. N-Hydroxysuccinimide (HOSu; CAS RN: 6066-82-6; 12 mg, 0.10 mmol) and EDCI (CAS RN: 7084-11-9; 20 mg, 0.10 mmol) were added to the reaction mixture and the reaction mixture was left stirring at 25° C. for 2 hours. Then a DMF solution (CAS RN: 68-12-2; 5 ml) containing intermediate L18aP2-h ((2S,3S,4S,5R,6S)-6-(2-(2-aminoacetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-3,4,5-trihydroxytetrahydro-2H-pyran-2-carboxylic acid; 93 mg, 0.10 mmol) and DIPEA (CAS RN: 7087-68-5; 25 mg, 0.19 mmol) was added to the reaction mixture. The reaction mixture was left stirring for another 3 hours at 25° C. LC-MS was used to confirm the full conversion of starting material in the reaction mixture. Formic acid was used to adjust the pH value of reaction mixture to 4.0. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L18aP2 as a yellow solid ((2S,3S,4S,5R,6S)-6-(2-(2-(2-((1-(2-(2-(2-(2-(2,5-dioxo-2,5-dihydro-1H-pyrrol-1-yl)acetamido)ethoxy)ethoxy)ethyl)-1H-1,2,3-triazol-4-yl)methoxy)acetamido)acetamido)-4-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenoxy)-3,4,5-trihydroxytetrahydro-2H-pyran-2-carboxylic acid; 16 mg, 0.012 mmol, 11.9% in yield after purification).

LC-MS (m/z, C60H70FN11O22Si)=1344.4 [M+H]⁺.

### Example 11: Linker-Payload L8rQP2

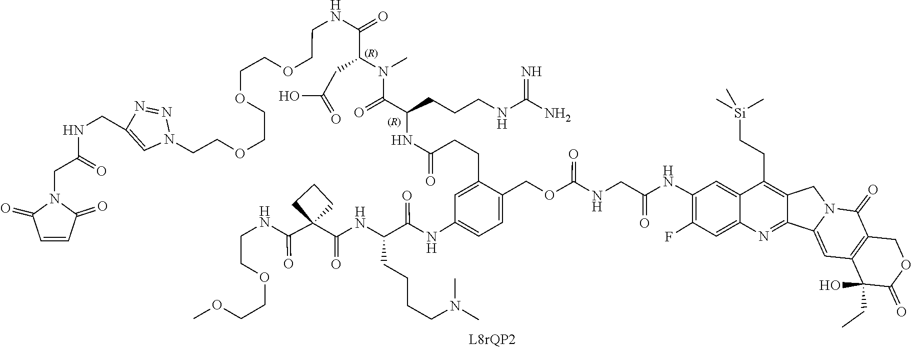

### Intermediate L8rQP2-a: tert-butyl((2-iodo-4-nitrobenzyl)oxy)dimethylsilane

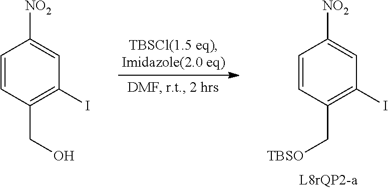 [SMILES: O=[N+]([O-])c1ccc(CO)c(I)c1 ; CC(C)(C)[Si](C)(C)OCc1ccc([N+](=O)[O-])cc1I]

(2-Iodo-4-nitro-phenyl)-methanol (CAS RN: 500563-90-6; 10 g, 35.84 mmol) was dissolved in DCM (CAS RN: 75-09-2; 250 mL) at room temperature. Then the reaction mixture was added with imidazole (CAS RN: 288-32-4; 5.80 g, 85.19 mmol) and tert-butyldimethylsilyl chloride (CAS RN: 18162-48-6, 9.80 g, 65.02 mmol). The reaction mixture was left stirring for 2 hours at room temperature. LC-MS was employed to verify the full consumption of starting material in the reaction mixture. The organic solvent in the reaction mixture was reduced in vacuo to afford the residue. The residue was then purified via preparative silica chromatography to afford the desirable intermediate L8rQP2-a (tert-butyl((2-iodo-4-nitrobenzyl)oxy)dimethylsilane; 11 g, 27.99 mmol, 78.09% in yield) as a white solid.

LC-MS (m/z, C13H20INO3Si)=394.4[M+H]⁺.

¹H NMR (400 MHz, DMSO-d₆) δ8.59 (d, J=2.4 Hz, 1H), 8.33 (dd, J=8.6, 2.4 Hz, 1H), 7.96-7.27 (m, 1H), 4.69 (s, 2H), 0.96 (s, 9H), 0.16 (s, 6H).

### Intermediate L8rQP2-b: tert-butyl 3-(2-(((tert-butyldimethylsilyl)oxy)methyl)-5-nitrophenyl)propiolate

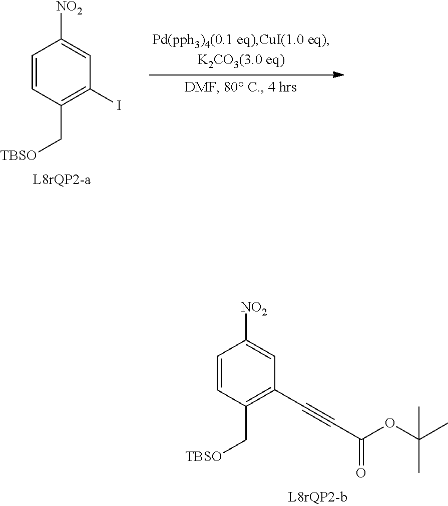 [SMILES: CC(C)(C)[Si](C)(C)OCc1ccc([N+](=O)[O-])cc1I ; CC(C)(C)OC(=O)C#Cc1cc([N+](=O)[O-])ccc1CO[Si](C)(C)C(C)(C)C]

Intermediate L8rQP2-a (tert-butyl((2-iodo-4-nitrobenzyl)oxy)dimethylsilane; 1.00 g, 2.54 mmol) was dissolved in DMF (CAS RN: 68-12-2; 200 mL). Then, tert-butyl propiolate (CAS RN: 13831-03-3; 1.60 g, 12.6 mmol), tetrakis(triphenylphosphine)palladium (CAS RN: 14221-01-3, 0.30 g, 0.26 mmol), copper iodide (CAS RN: 7681-65-4; 0.5 g, 2.63 mmol) and potassium carbonate (CAS RN: 584-08-7; 1.00 g, 7.24 mmol) were added to the reaction mixture at room temperature. Then the reaction mixture was heated to 80° C. and left stirring for 4 hours. LC-MS was used to confirm the full consumption of starting material in the reaction mixture. The organic solvent in the reaction mixture was reduced in vacuo to afford the reaction residue. The residue was then purified via silica chromatography to afford the desirable intermediate L8rQP2-b (tert-butyl 3-(2-(((tert-butyldimethylsilyl)oxy)methyl)-5-nitrophenyl)propiolate; 0.8 g, 2.05 mmol, 80.70% in yield) as a white solid.

LC-MS (m/z, C20H29NO5Si)=392.5 [M+H]⁺.

¹H NMR (400 MHz, DMSO-d₆) δ 8.41 (m, 2H), 7.87-7.74 (m, 1H), 4.92 (s, 2H), 1.52 (s, 9H), 0.94 (s, 9H), 0.14 (s, 6H).

### Intermediate L8rQP2-c: tert-butyl 3-(5-amino-2-(((tert-butyldimethylsilyl)oxy)methyl)phenyl)propanoate

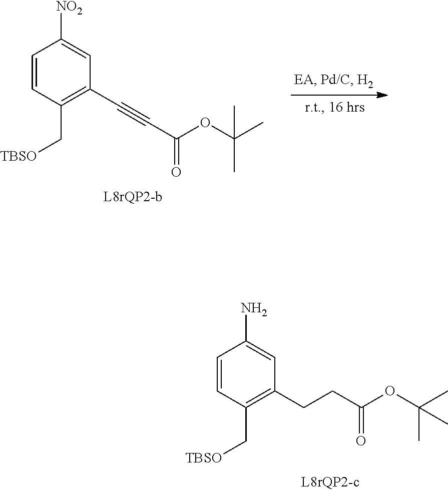 [SMILES: CC(C)(C)OC(=O)C#Cc1cc([N+](=O)[O-])ccc1CO[Si](C)(C)C(C)(C)C ; CC(C)(C)OC(=O)CCc1cc(N)ccc1CO[Si](C)(C)C(C)(C)C]

Intermediate L8rQP2-b (tert-butyl 3-(2-(((tert-butyldimethylsilyl)oxy)methyl)-5-nitrophenyl)propiolate; 800 mg, 2.04 mmol) was dissolved in ethyl acetate (CAS RN: 141-78-6; 10 mL) at room temperature. Then, palladium on carbon (CAS RN: 7440-05-3; 100 mg) was added to the reaction mixture and the mixture was stirred at room temperature under hydrogen atmosphere for 16 hours. LC-MS was used to confirm the full consumption of starting materials in the reaction mixture. The organic solvent in the reaction mixture was reduced in vacuo to afford the reaction residue. The residue was then purified via preparative silica chromatography (petroleum ether:ethyl acetate=9:1) to afford the desirable intermediate L8rQP2-c (tert-butyl 3-(5-amino-2-(((tert-butyldimethylsilyl)oxy)methyl)phenyl)propanoate; 600 mg, 1.64 mmol, 80.39% in yield) as a white solid.

LC-MS (m/z, C20H35NO3Si)=388.2[M+Na]⁺.

¹H NMR (400 MHz, DMSO) δ 6.91 (d, J=8.0 Hz, 1H), 6.49-6.26 (m, 2H), 4.97 (s, 2H), 4.52 (s, 2H), 2.73 (m, 2H), 2.46 (m, 2H), 1.40 (s, 9H), 0.88 (s, 9H), 0.05 (s, 6H).

### Intermediate L8rQP2-d: tert-butyl (S)-3-(5-(2-((((9H-fluoren-9-yl)methoxy)carbonyl)amino)-6-(dimethylamino)hexanamido)-2-(((tert-butyldimethylsilyl)oxy)methyl)phenyl)propanoate

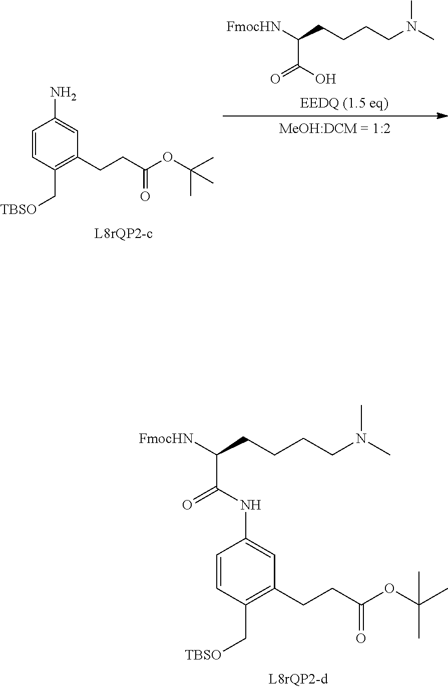

Intermediate L8rQP2-c (tert-butyl 3-(5-amino-2-(((tert-butyldimethylsilyl)oxy)methyl)phenyl)propanoate; 3.3 g, 9.03 mmol) was dissolved in a mixture of methanol (CAS RN: 67-56-1; 10 ml) and DCM (CAS RN: 75-09-2; 20 ml). Then N-ethoxycarbonyl-2-ethoxy-1,2-dihydroquinoline (CAS RN: 16357-59-8, 2.79 g, 11.28 mmol) and N²-Fmoc-N⁶,N⁶-dimethyl-L-lysine (CAS RN: 378802-00-7, 3.58 g, 9.03 mmol) were added to the reaction mixture and the reaction mixture was stirred for another 3 hours at room temperature. LC-MS was employed to verify the full consumption of starting materials in the reaction mixture. The organic solvent in the reaction mixture was reduced in vacuo to afford the residue. The residue was then purified via preparative silica chromatography (DCM:methanol=2:1) to afford the desirable intermediate L8rQP2-d (tert-butyl (S)-3-(5-(2-((((9H-fluoren-9-yl)methoxy)carbonyl)amino)-6-(dimethylamino)hexanamido)-2-(((tert-butyldimethylsilyl)oxy)methyl)phenyl)propanoate; 3.00 g, 4.03 mmol, 44.63% in yield) as a light yellow oil.

LC-MS (m/z, C₄₃H₆₁N₃O₆Si)=744.4[M+H]⁺.

### Intermediate L8rQP2-e: tert-butyl (S)-3-(5-(2-((((9H-fluoren-9-yl)methoxy) carbonyl)amino)-6-(dimethylamino)hexanamido)-2-(hydroxymethyl)phenyl)propanoate

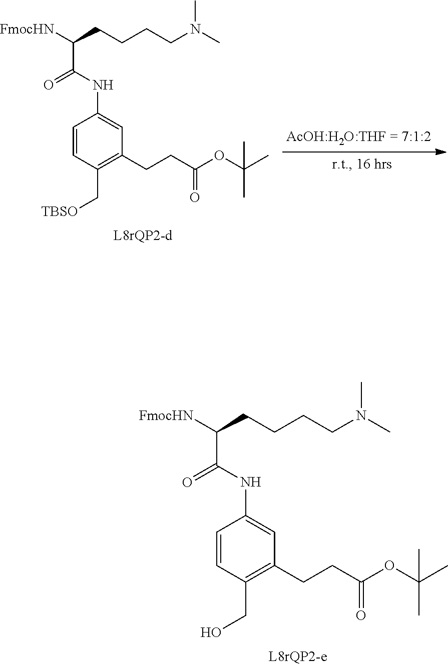

Intermediate L8rQP2-d (tert-butyl (S)-3-(5-(2-((((9H-fluoren-9-yl)methoxy)carbonyl)amino)-6-(dimethylamino)hexanamido)-2-(((tert-butyldimethylsilyl)oxy)methyl)phenyl)propanoate; 3.00 g, 4.03 mmol) was dissolved in a mixture of acetic acid (CAS RN: 64-19-7; 21 ml), water (3 ml) and THF (CAS RN: 109-99-9; 6 ml) at room temperature. The reaction mixture was stirred at room temperature for 18 hours. Samples of reaction mixture were taken to be analyzed by LC-MS to confirm the full consumption of starting materials. The organic solvent in the reaction mixture was reduced in vacuo to afford the residue. The residue was then purified via preparative reverse-phase liquid chromatography (Column: Welch-Ultimate-C18-10 μm-21.2*150 mm, mobile phase A: 0.1% TFA in H₂O; mobile phase B: ACN) to afford the desirable intermediate L8rQP2-e (tert-butyl (S)-3-(5-(2-((((9H-fluoren-9-yl)methoxy)carbonyl)amino)-6-(dimethylamino)hexanamido)-2-(hydroxymethyl)phenyl)propanoate; 1.00 g, 1.59 mmol, 39.38% in yield) as a white solid.

LC-MS (m/z, C₃₇H₄₇N₃O₆)=630.4[M+H]⁺.

### Intermediate L8rQP2-f: tert-butyl (S)-3-(5-(2-((((9H-fluoren-9-yl)methoxy)carbonyl)amino)-6-(dimethylamino)hexanamido)-2-((((4-nitrophenoxy)carbonyl)oxy)methyl)phenyl)propanoate

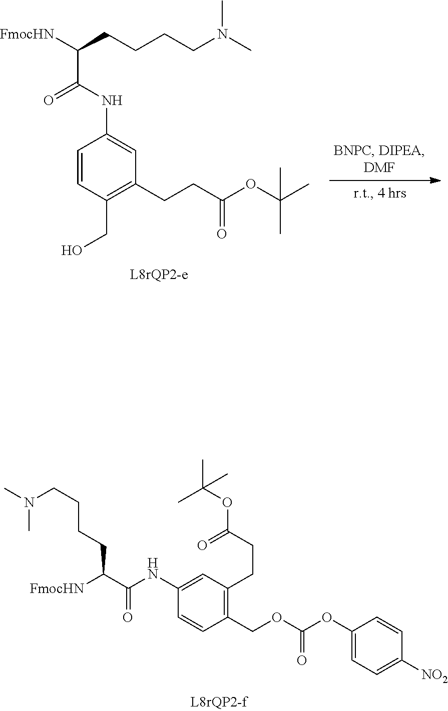

Intermediate L8rQP2-e (tert-butyl (S)-3-(5-(2-((((9H-fluoren-9-yl) methoxy) carbonyl) amino)-6-(dimethylamino)hexanamido)-2-(hydroxymethyl)phenyl)propanoate; 920 mg, 1.46 mmol) was dissolved in DMF (CAS RN: 68-12-2; 10 ml) at room temperature. Then DIPEA (CAS RN: 7087-68-5; 377 mg, 2.92 mmol) and bis(4-nitrophenyl)carbonate (BNPC; CAS RN: 5070-13-3; 1110 mg, 3.65 mmol) were dissolved in the reaction mixture and the reaction mixture was left stirring at room temperature for 4 hours. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-f (tert-butyl (S)-3-(5-(2-((((9H-fluoren-9-yl)methoxy)carbonyl)amino)-6-(dimethylamino)hexanamido)-2-((((4-nitrophenoxy)carbonyl)oxy)methyl)phenyl)propanoate; 610 mg, 0.77 mmol, 52.53% in yield) as a white solid.

LC-MS (m/z, C₄₄H₅₀N₄O₁₀)=795.4[M+H]⁺.

¹H NMR (DMSO-d₆, 400 MHz) δ 10.13 (s, 1H), 9.38 (s, 1H), 8.33 (m, 2H), 7.92 (m, 2H), 7.7-7.8 (m, 3H), 7.5-7.6 (m, 4H), 7.3-7.5 (m, 4H), 5.34 (s, 2H), 4.3-4.4 (m, 2H), 4.2-4.3 (m, 1H), 4.1-4.2 (m, 1H), 3.0-3.1 (m, 2H), 2.92 (m, 2H), 2.77 (m, 6H), 2.5-2.6 (m, 2H), 1.6-1.7 (m, 4H), 1.4-1.4 (m, 9H), 1.3-1.4 (m, 2H).

### Intermediate L8rQP2-g: tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate

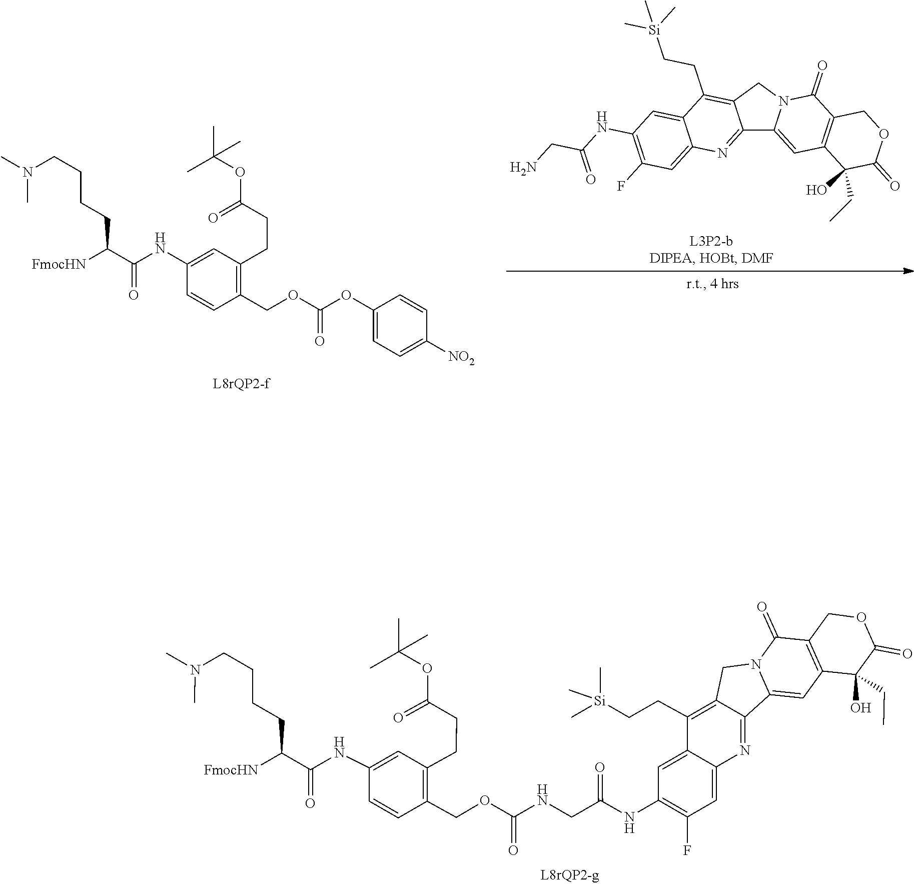

Intermediate L8rQP2-f (tert-butyl (S)-3-(5-(2-((((9H-fluoren-9-yl)methoxy)carbonyl)amino)-6-(dimethylamino)hexanamido)-2-((((4-nitrophenoxy)carbonyl)oxy)methyl)phenyl)propanoate; 600 mg, 0.75 mmol) and intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 488 mg, 0.91 mmol) was dissolved in DMF (CAS RN: 68-12-2; 10 mL) at room temperature. Then, DIPEA (CAS RN: 7087-68-5; 156 mg, 1.21 mmol) and HOBt (CAS RN: 2592-95-2; 102 mg, 0.75 mmol) were added to the reaction mixture and the reaction mixture was stirred at room temperature for another 4 hours. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-g (tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 610 mg, 0.51 mmol, 50.96% in yield) as a yellow solid.

LC-MS (m/z, C₆₅H₇₆FN₇O₁₂Si)=597.8 [1/2M+H]⁺.

### Intermediate L8rQP2-h: 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid

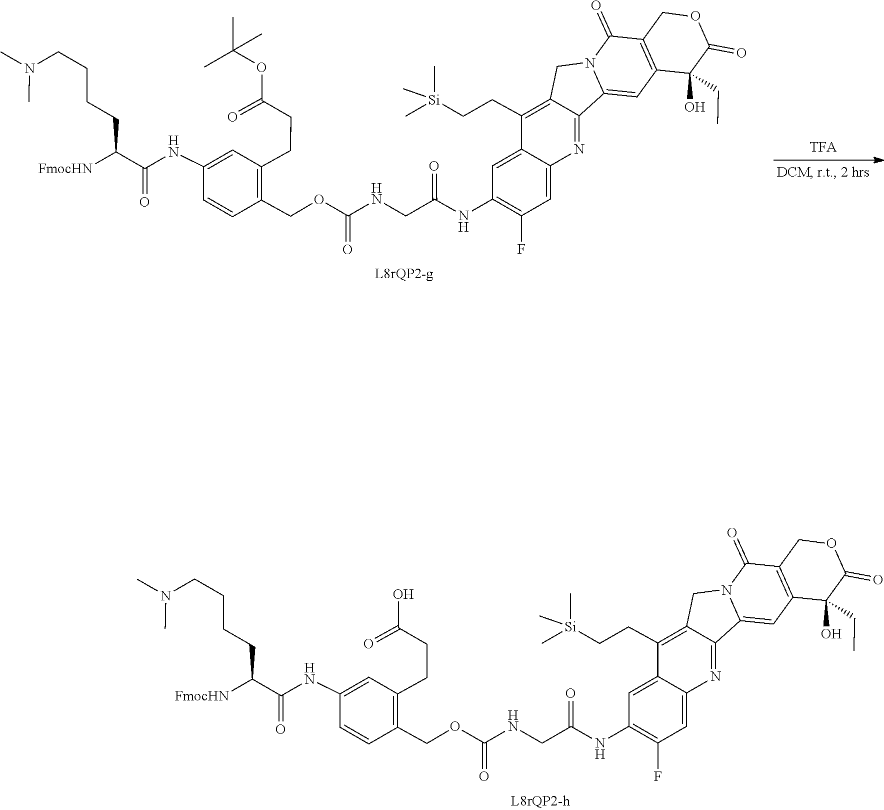

Intermediate L8rQP2-g (tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanoate; 400 mg, 0.33 mmol) was dissolved in DCM (CAS RN: 75-09-2; 10 mL). Then, TFA (CAS RN: 76-05-1; 3 mL) was added to the reaction mixture. The reaction mixture was stirred at room temperature for another 3 hours. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-h (3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 200 mg, 0.176 mmol, 52.46% in yield) as a yellow solid.

LC-MS (m/z, C₆₁H₆₈FN₇O₁₂Si)=1138.4 [M+H]⁺.

### Intermediate L8rQP2-i: tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

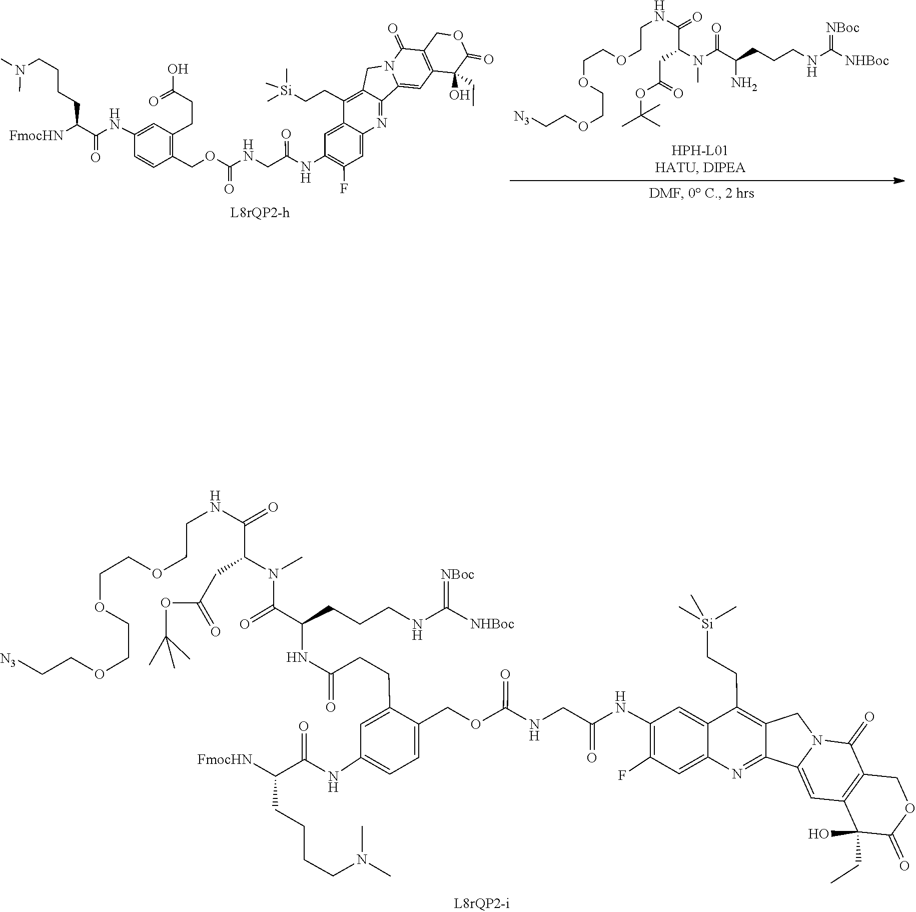

Intermediate L8rQP2-h (3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 35 mg, 0.03 mmol) was dissolved in DMF (CAS RN: 68-12-2; 2 mL) at 0° C. Then, DIPEA (CAS RN: 7087-68-5, 8 mg, 0.06 mmol), HATU (CAS RN: 148893-10-1, 18 mg, 0.05 mmol) and intermediate HPH-L01 (tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 20 mg, 0.03 mmol) were added to the reaction mixture, and the reaction mixture was left stirring at 0° C. for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-i (tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 20 mg, 0.01 mmol, 36.53% in yield) as a yellow solid.

LC-MS (m/z, C₉₄H₁₂₇FN₁₆O₂₂Si)=940.8[M/2+H]⁺.

### Intermediate L8rQP2-j: tert-butyl (11R,14R,E)-11-(3-(5-((S)-2-amino-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

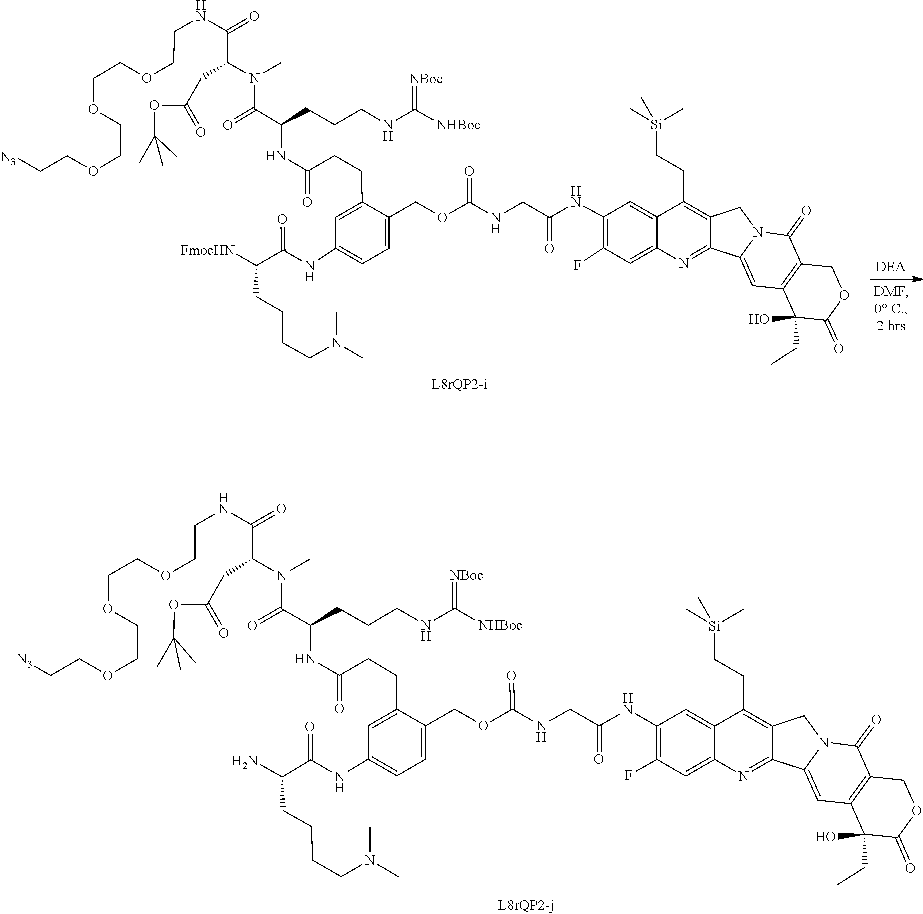

Intermediate L8rQP2-i (tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 20 mg, 0.01 mmol) was dissolved in DMF (CAS RN: 68-12-2; 2 mL) at 0° C. Then, diethylamine (CAS RN: 109-89-7, 0.02 mL, 0.11 mmol) was added to the reaction mixture, and the reaction mixture was left stirring at 0° C. for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-j (tert-butyl (11R,14R,E)-11-(3-(5-((S)-2-amino-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 10 mg, 0.0053 mmol, 53.00% in yield) as a yellow solid.

LC-MS (m/z, C₇₉H₁₁₇FN₁₆O₂₀Si)=829.6[M/2+H]⁺.

### Intermediate L8rQP2-k: tert-butyl (11R,14R,E)-14-((azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

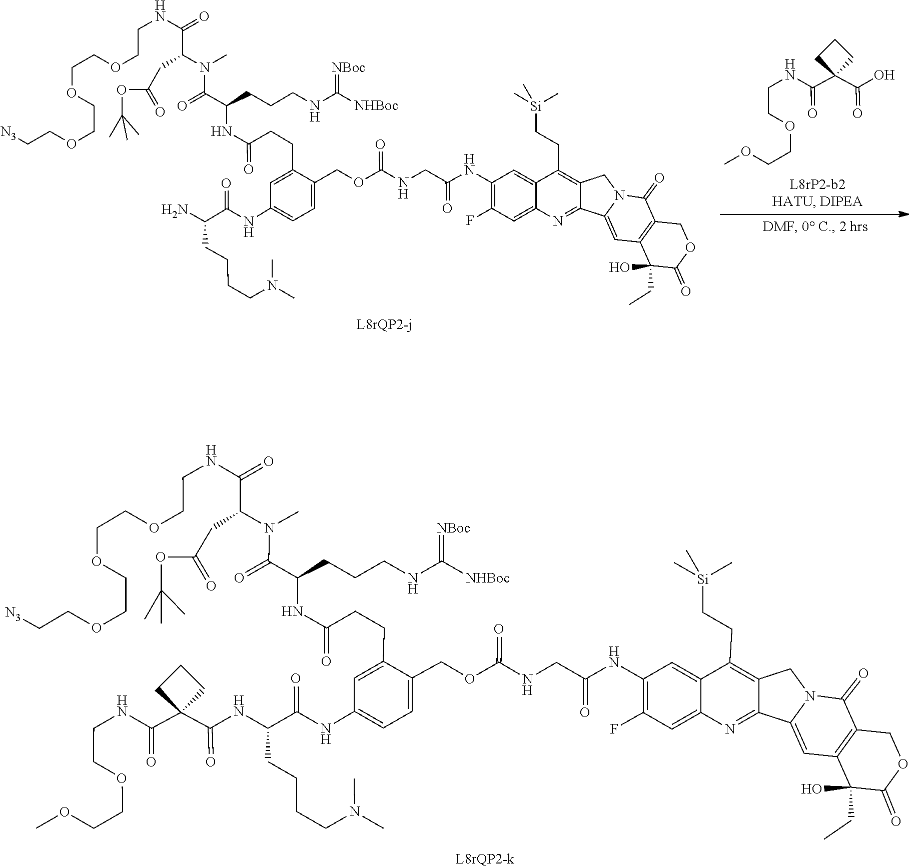

Intermediate L8rP2-b2 (1-((2-(2-methoxyethoxy)ethyl)carbamoyl)cyclobutane-1-carboxylic acid; 3 mg, 0.01 mol) was dissolved in DMF (CAS RN: 68-12-2; 1 ml) at 0° C. Then, DIPEA (CAS RN: 7087-68-5, 2 mg, 0.02 mmol) and HATU (CAS RN:148893-10-1, 4 mg, 0.01 mmol) were added to the reaction mixture, and the reaction mixture was stirred at 0° C. for 2 hours before the addition of intermediate L8rQP2-j (tert-butyl (11R,14R,E)-11-(3-(5-((S)-2-amino-6-(dimethylamino)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 10 mg, 0.00603 mmol). The reaction mixture was left stirring at 0° C. for additional 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-k (tert-butyl (11R,14R,E)-14-((azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 5 mg, 0.00265 mmol, 43.95% in yield) as a white solid.

LC-MS (m/z, C₉₀H₁₃₄FN₁₇O₂₄Si)=943.0[M/2+H]⁺.

### Intermediate L8rQP2-1: tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

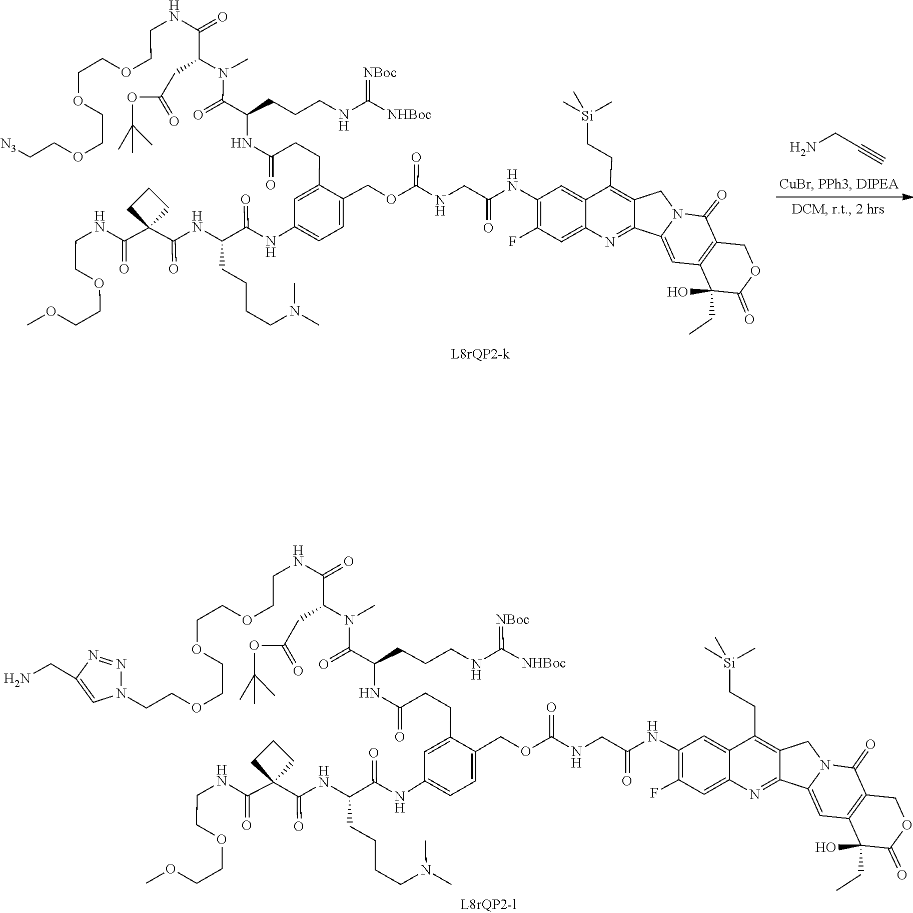

Intermediate L8rQP2-k (tert-butyl (11R,14R,E)-14-((azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 20 mg, 0.0106 mmol) was dissolved in DCM (CAS RN: 75-09-2; 2 mL) at room temperature. Then, triphenylphosphine (CAS RN:603-35-0; 3 mg, 0.01 mmol), DIPEA (CAS RN: 7087-68-5; 3 mg, 0.02 mmol), propargylamine (CAS RN: 2450-71-7; 3 mg, 0.05 mmol) and copper(I) bromide (CAS RN:7787-70-4; 3 mg, 0.02 mmol) were added to the reaction mixture, and the reaction mixture was stirred at room temperature for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-1 (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 10 mg, 0.00515 mmol, 48.58% in yield) as a white solid.

### Intermediate L8rQP2-m: tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate

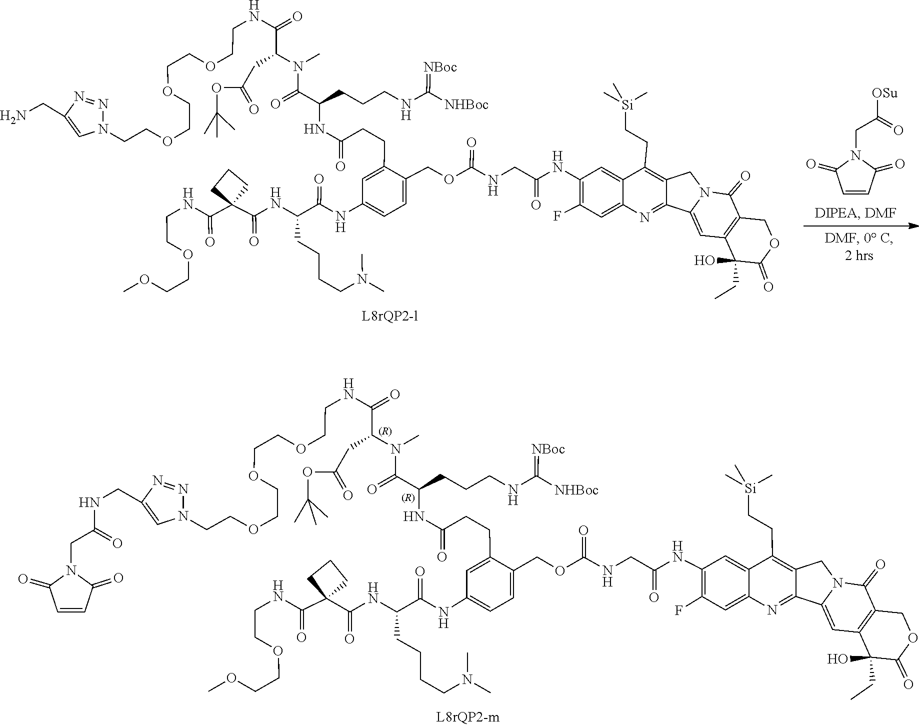

Intermediate L8rQP2-1 (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 120 mg, 0.0618 mmol) was dissolved in DMF (CAS RN: 68-12-2; 5 mL) at 0° C. Then, DIPEA (CAS RN: 7087-68-5; 17.00 mg, 0.13 mmol) and N-succinimidyl maleimidoacetate (CAS RN: 55750-61-3, 25 mg, 0.10 mmol) were added to the reaction mixture, and the reaction mixture was further stirred at 0° C. for another 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rQP2-m (tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 80 mg, 0.0385 mmol, 62.30% in yield) as a white solid.

LC-MS (m/z, C₉₉H₁₄₂FN₁₉O₂₇Si)=1039.3 [M/2+H]⁺.

### Linker-payload L8rQP2: (R)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

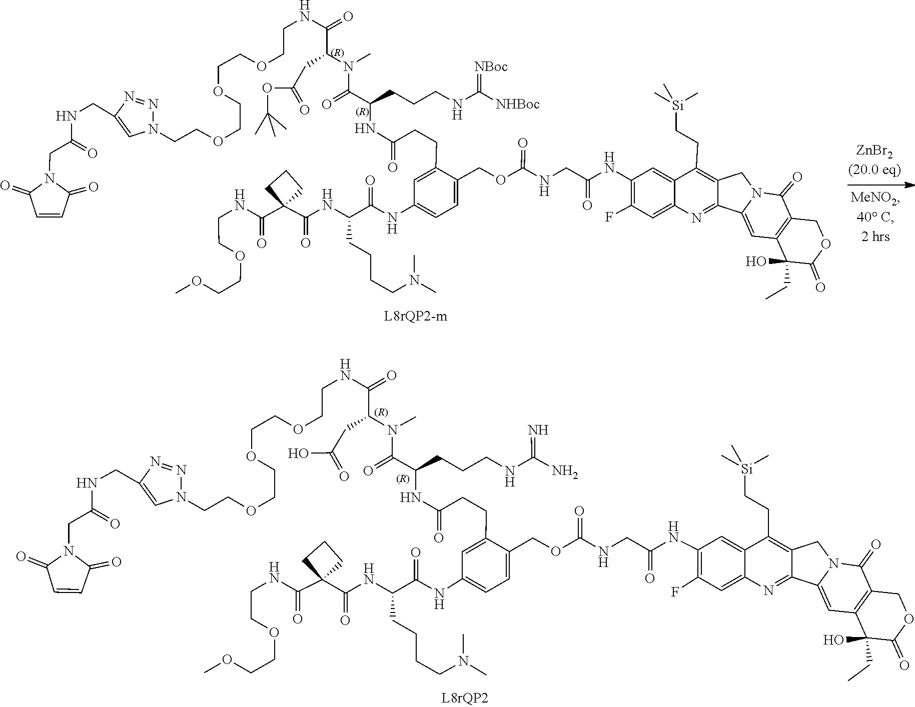

Intermediate L8rQP2-m (tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 80 mg, 0.0385 mmol) was dissolved in nitromethane (CAS RN: 75-52-5; 5 ml) at 40° C. Then, zinc bromide (CAS RN: 7699-45-8, 184 mg, 0.82 mmol) was added to the reaction mixture, and the reaction mixture was stirred for 2 hours at 40° C. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L8rQP2 ((R)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 8.89 mg, 0.0049 mmol, 12.68% in yield after purification) as a white solid.

LC-MS (m/z, C₈₅H₁₁₈FN₁₉O₂₃Si)=911.2[M/2+H]⁺.

### Example 12: Linker-Payload L8rRP2

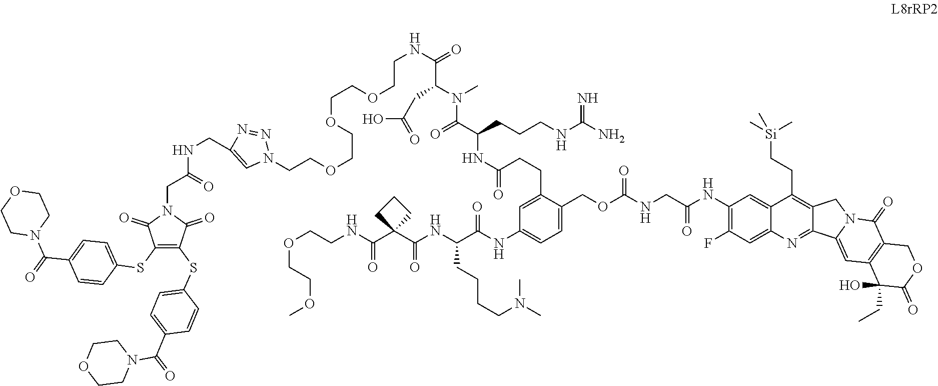

### Intermediate L8rRP2-a: tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

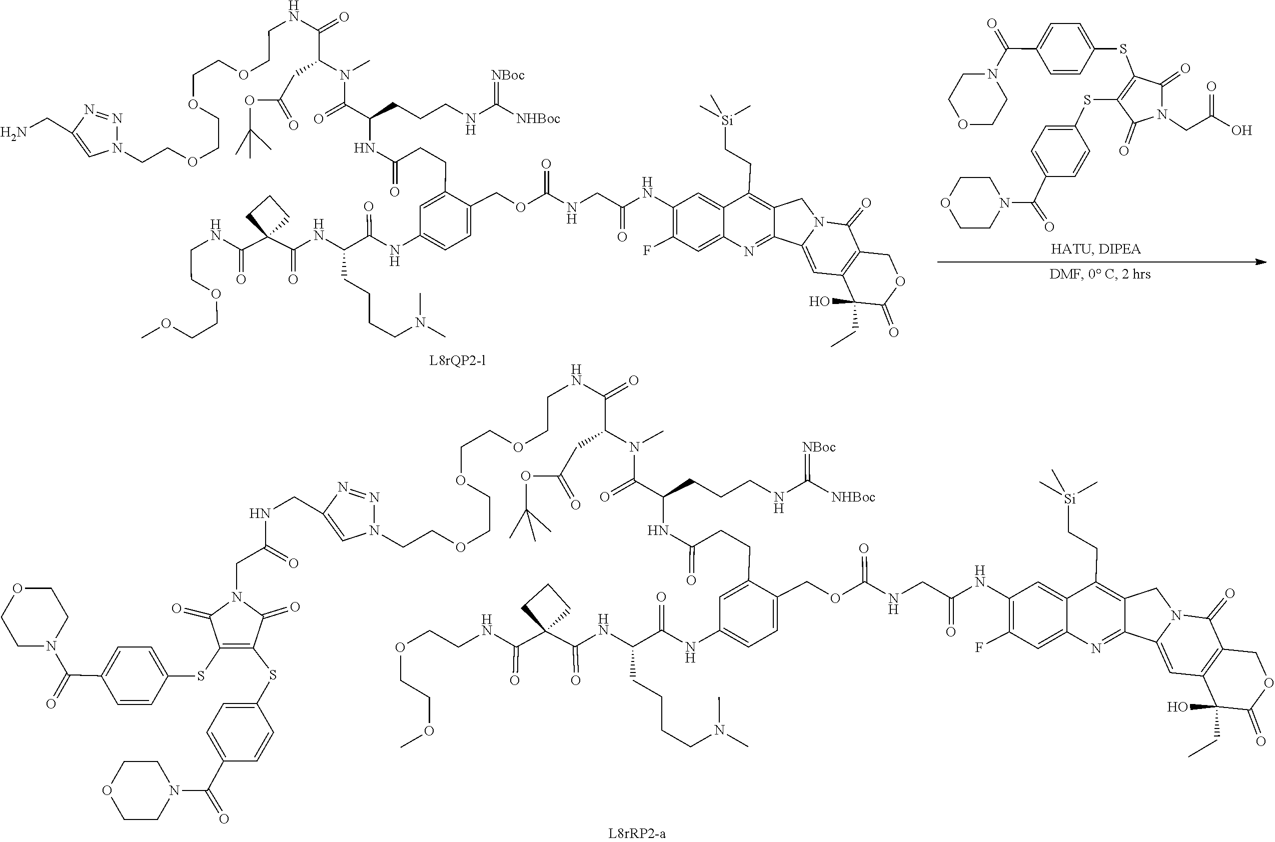

Intermediate L8rQP2-1 (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 120 mg, 0.0618 mmol) was dissolved in DMF (CAS RN: 68-12-2; 5 mL) at 0° C. Then, DIPEA (CAS RN: 7087-68-5, 17.00 mg, 0.13 mmol), HATU (CAS RN: 148893-10-1; 38 mg, 0.10 mmol) and intermediate DSRB-C01 (2-(3,4-bis((4-(morpholine-4-carbonyl)phenyl)thio)-2,5-dioxo-2,5-dihydro-1H-pyrrol-1-yl)acetic acid; 59 mg, 0.10 mmol) were added to the reaction mixture, and the reaction mixture was further stirred at 0° C. for another 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rRP2-a (tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 80 mg, 0.0317 mmol, 51.39% in yield) as a yellow solid.

LC-MS (m/z, C₁₂₁H₁₆₄FN₂₁O₃₁S₂Si)=1260.5 [M/2+H]⁺.

### Linker-payload L8rRP2: (R)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-13-oxo-3,6,9-trioxa-12-azahexadecan-16-oic acid

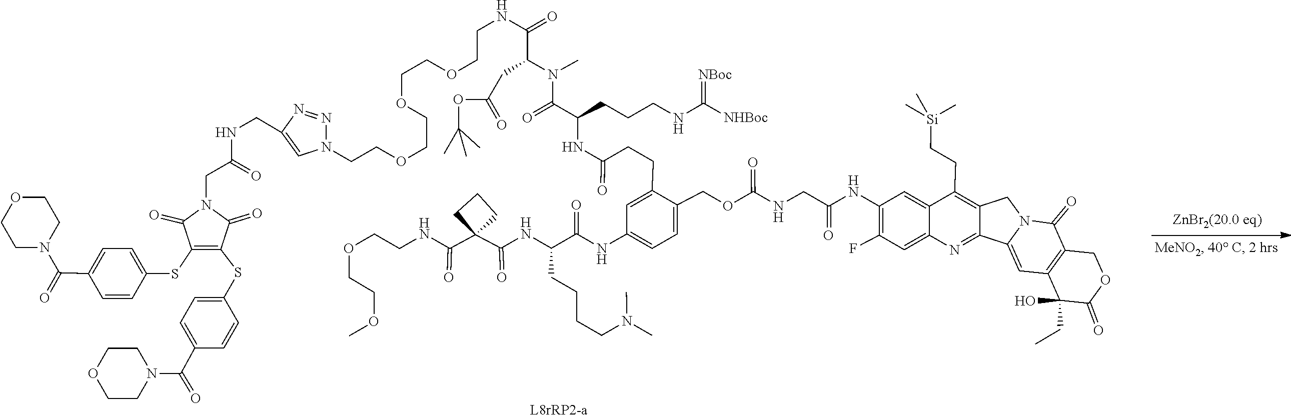

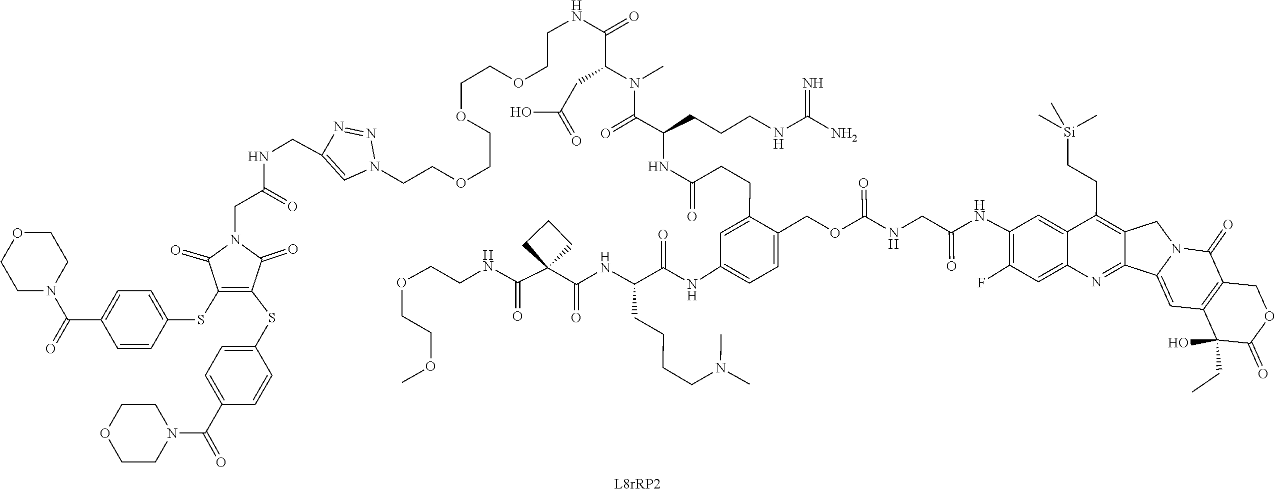

Intermediate L8rRP2-a (tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 80 mg, 0.0317 mmol) was dissolved in nitromethane (CAS RN: 75-52-5; 5 ml) at 40° C. Then, zinc bromide (CAS RN: 7699-45-8, 150 mg, 0.67 mmol) was added to the reaction mixture, and the reaction mixture was stirred for 2 hours at 40° C. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L8rRP2 ((R)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-13-oxo-3,6,9-trioxa-12-azahexadecan-16-oic acid; 12.72 mg, 0.00562 mmol, 17.73% in yield after purification) as a yellow solid.

LC-MS (m/z, C₁₀₇H₁₄₀FN₂₁O₂₇S₂Si)=1132.3[M/2+H]⁺.

### Example 13: Linker-payload L8rSP2

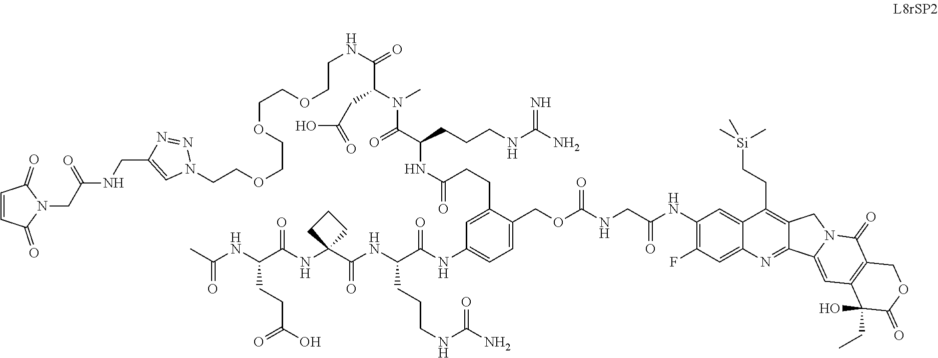

### Intermediate L8rSP2-a: tert-butyl (S)-3-(5-(2-((((9H-fluoren-9-yl)methoxy)carbonyl)amino)-5-ureidopentanamido)-2-(((tert-butyldimethylsilyl)oxy)methyl)phenyl)propanoate

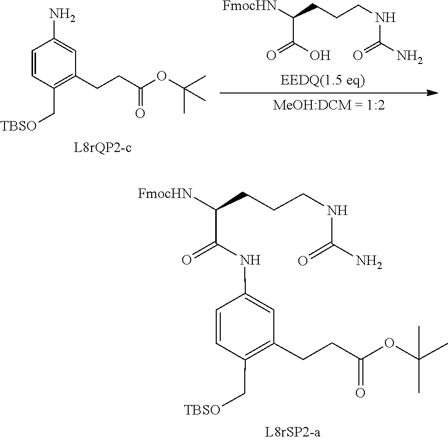

Intermediate L8rQP2-c (tert-butyl 3-(5-amino-2-(((tert-butyldimethylsilyl)oxy)methyl)phenyl)propanoate; 0.7 g, 1.92 mmol) was dissolved in a mixture of methanol (CAS RN: 67-56-1; 6 ml) and DCM (CAS RN: 75-09-2; 3 ml). Then N-ethoxycarbonyl-2-ethoxy-1,2-dihydroquinoline (CAS RN: 16357-59-8, 597 mg, 2.41 mmol) and Fmoc-L-citrulline (CAS RN: 133174-15-9; 767 mg, 1.93 mmol) were added to the reaction mixture and the reaction mixture was stirred for another 3 hours at room temperature. LC-MS was employed to verify the full consumption of starting materials in the reaction mixture. The organic solvent in the reaction mixture was reduced in vacuo to afford the residue. The residue was then purified via preparative silica chromatography (DCM:methanol=2:1) to afford the desirable intermediate L8rSP2-a (tert-butyl (S)-3-(5-(2-((((9H-fluoren-9-yl)methoxy)carbonyl)amino)-5-ureidopentanamido)-2-(((tert-butyldimethylsilyl)oxy)methyl)phenyl)propanoate; 800 mg, 1.07 mmol, 55.73% in yield) as a yellow oil.

LC-MS (m/z, C₄₁H₅₆N₄O₇Si)=N/A.

¹H NMR (400 MHz, DMSO-d₆) δ 9.97 (s, 1H), 7.91 (d, J=7.6 Hz, 2H), 7.76 (dd, J=7.6, 3.5 Hz, 2H), 7.68 (d, J=8.0 Hz, 1H), 7.55-7.42 (m, 4H), 7.35 (dt, J=9.2, 4.6 Hz, 2H), 7.31-7.20 (m, 1H), 6.00 (s, 1H), 5.44 (s, 2H), 4.65 (s, 2H), 4.26 (m, 1H), 4.20-4.09 (m, 2H), 3.00 (m, 2H), 2.82 (m, 2H), 2.49 (m, 2H) 1.65 (s, 2H), 1.39 (m, 11H), 0.89 (s, 9H), 0.08 (s, 6H).

### Intermediate L8rSP2-b: tert-butyl (S)-3-(5-(2-((((9H-fluoren-9-yl)methoxy)carbonyl)amino)-5-ureidopentanamido)-2-(hydroxymethyl)phenyl)propanoate

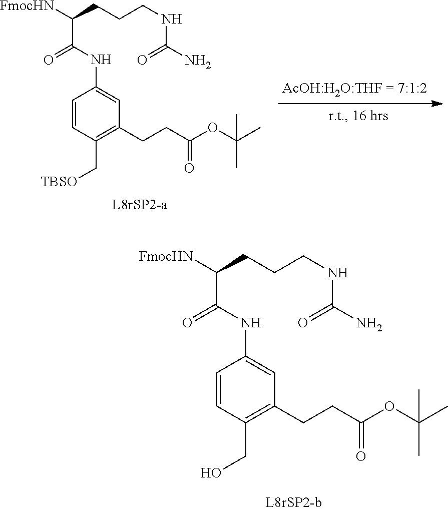

Intermediate L8rSP2-a (tert-butyl (S)-3-(5-(2-((((9H-fluoren-9-yl)methoxy)carbonyl)amino)-5-ureidopentanamido)-2-(((tert-butyldimethylsilyl)oxy)methyl)phenyl)propanoate; 800 mg, 0.98 mmol) was dissolved in a mixture of acetic acid (CAS RN: 64-19-7; 21 ml), water (3 ml) and THF (CAS RN: 109-99-9; 6 ml) at room temperature. The reaction mixture was stirred at room temperature for 16 hours. Samples of reaction mixture were taken to be analyzed by LC-MS to confirm the full consumption of starting materials. The organic solvent in the reaction mixture was reduced in vacuo to afford the residue. The residue was then purified via preparative reverse-phase liquid chromatography (Column: Welch-Ultimate-C18-10 μm-21.2*150 mm, mobile phase A: 0.1% TFA in H₂O; mobile phase B: ACN) to afford the desirable intermediate L8rSP2-b (tert-butyl (S)-3-(5-(2-((((9H-fluoren-9-yl)methoxy)carbonyl)amino)-5-ureidopentanamido)-2-(hydroxymethyl)phenyl)propanoate; 500 mg, 0.7923 mmol, 73.78% in yield) as a white solid.

LC-MS (m/z, C₃₅H₄₂N₄O₇)=632.4[M+H]⁺.

¹H NMR (400 MHz, DMSO-d₆) δ 9.95 (s, 1H), 7.91 (m, 2H), 7.77 (m, 2H), 7.67 (m, 1H), 7.50-7.41 (m, 4H), 7.35 (m, 2H), 7.26 (m, 1H), 6.01 (s, 1H), 5.44 (s, 2H), 5.02 (t, J=5.2 Hz, 1H), 4.47 (m, 2H), 4.35-4.12 (m, 4H), 3.09-2.94 (m, 2H), 2.83 (t, J=7.6 Hz, 2H), 1.78-1.55 (m, 2H), 1.50-1.30 (m, 11H).

### Intermediate L8rSP2-c: tert-butyl (S)-3-(5-(2-((((9H-fluoren-9-yl)methoxy)carbonyl)amino)-5-ureidopentanamido)-2-((((4-nitrophenoxy)carbonyl)oxy)methyl)phenyl)propanoate

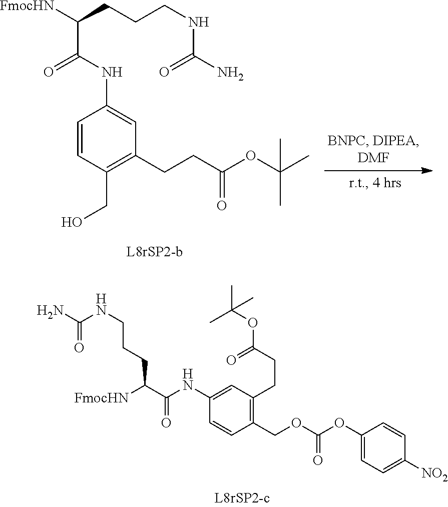

Intermediate L8rSP2-b (tert-butyl (S)-3-(5-(2-((((9H-fluoren-9-yl)methoxy)carbonyl)amino)-5-ureidopentanamido)-2-(hydroxymethyl)phenyl)propanoate; 460 mg, 0.73 mmol) was dissolved in DMF (CAS RN: 68-12-2; 10 ml) at room temperature. Then DIPEA (CAS RN: 7087-68-5; 179 mg, 1.38 mmol) and bis(4-nitrophenyl)carbonate (BNPC; CAS RN: 5070-13-3; 317 mg, 1.04 mmol) were dissolved in the reaction mixture and the reaction mixture was left stirring at room temperature for 4 hours. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-c (tert-butyl (S)-3-(5-(2-((((9H-fluoren-9-yl)methoxy)carbonyl)amino)-5-ureidopentanamido)-2-((((4-nitrophenoxy)carbonyl)oxy)methyl)phenyl)propanoate; 230 mg, 0.289 mmol, 39.58% in yield) as a white solid.

LC-MS (m/z, C₄₂H₄₅N₅O₁₁)=740.2 [M−55]⁺.

¹H NMR (400 MHz, DMSO-d₆) δ 10.11 (s, 1H), 8.33 (d, J=8.8 Hz, 2H), 7.92 (d, J=7.6 Hz, 2H), 7.79-7.75 (m, 2H), 7.71 (d, J=7.6 Hz, 1H), 7.58 (m, 3H), 7.52 (m, 1H), 7.44 (m, 2H), 7.37 (3, 2H), 6.02 (s, 1H), 5.45 (bs, 2H), 5.33 (s, 2H), 4.35-4.11 (m, 4H), 3.06-2.87 (m, 4H), 2.5 (m, 2H), 1.63 (m, 2H), 1.37 (s, 11H).

### Intermediate L8rSP2-d: tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate

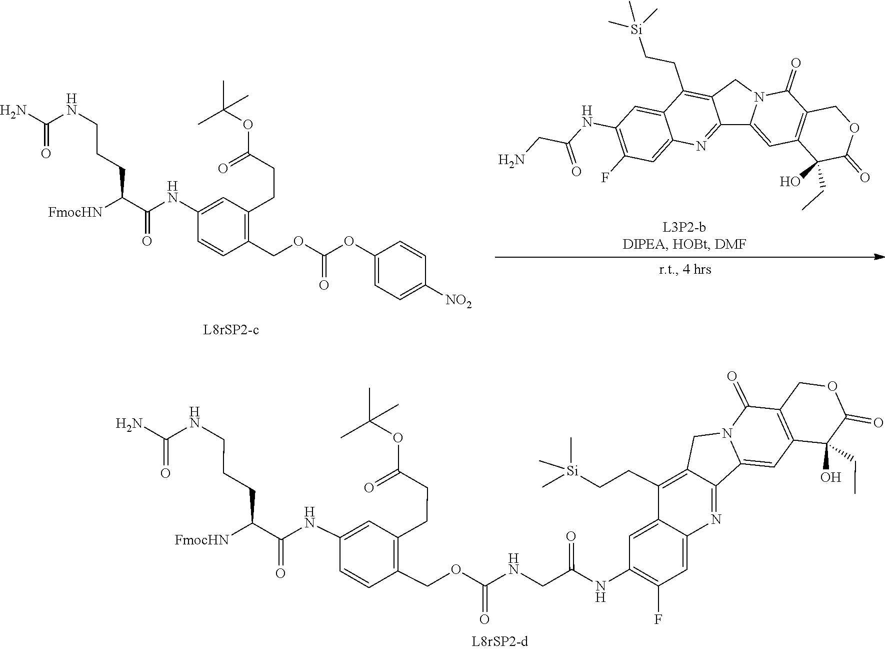

Intermediate L8rSP2-c (tert-butyl (S)-3-(5-(2-((((9H-fluoren-9-yl)methoxy)carbonyl)amino)-5-ureidopentanamido)-2-((((4-nitrophenoxy)carbonyl)oxy)methyl)phenyl)propanoate; 1.50 g, 1.88 mmol) and intermediate L3P2-b ((S)-2-amino-N-(4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)acetamide; 1.02 g, 1.88 mmol) were dissolved in DMF (CAS RN: 68-12-2; 8 mL) at room temperature. Then, DIPEA (CAS RN: 7087-68-5; 370 mg, 2.86 mmol) and HOBt (CAS RN: 2592-95-2; 380 mg, 2.81 mmol) were added to the reaction mixture and the reaction mixture was stirred at room temperature for another 4 hours. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-d (tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 1.92 g, 1.61 mmol, 85.43% in yield) as a yellow solid.

LC-MS (m/z, C₆₃H₇₁FN₈O₁₃Si)=1195.2[M+H]⁺.

### Intermediate L8rSP2-e: 3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid

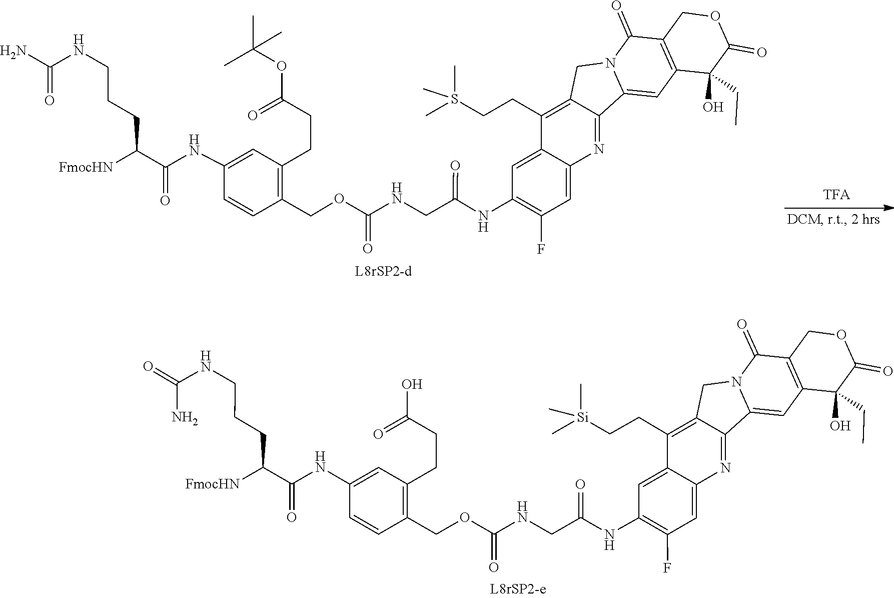

Intermediate L8rSP2-d (tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 1.92 g, 1.61 mmol) was dissolved in DCM (CAS RN: 75-09-2; 8 mL). Then, TFA (CAS RN: 76-05-1; 2.6 mL) was added to the reaction mixture. The reaction mixture was stirred at room temperature for another 3 hours. LC-MS was used to confirm the full conversion of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-e (3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 825 mg, 0.72 mmol, 45.00% in yield) as a yellow solid.

LC-MS (m/z, C₅₉H₆₃FN₈O₁₃Si)=1139.4[M+H]⁺.

### Intermediate L8rSP2-f: tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

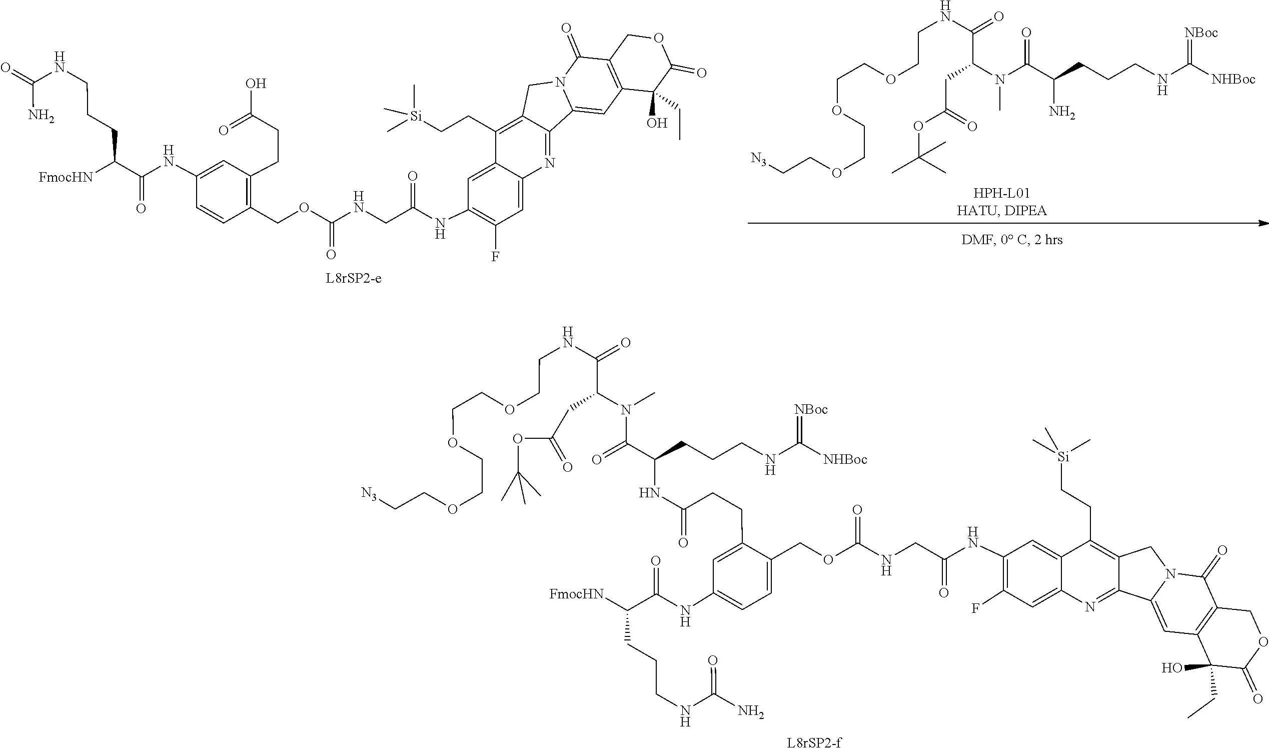

Intermediate L8rSP2-e (3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 825 mg, 0.72 mmol) was dissolved in DMF (CAS RN: 68-12-2; 5 mL) at 0° C. Then, DIPEA (CAS RN: 7087-68-5, 141 mg, 1.09 mmol), HATU (CAS RN: 148893-10-1, 414 mg, 1.09 mmol) and intermediate HPH-L01 (tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 551 mg, 0.73 mmol) were added to the reaction mixture, and the reaction mixture was left stirring at 0° C. for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-f (tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 742 mg, 0.39 mmol, 54.82% in yield) as a yellow solid.

LC-MS (m/z, C₉₂H₁₂₂FN₁₇O₂₃Si)=941.0[M/2+H]⁺.

### Intermediate L8rSP2-g: tert-butyl (11R,14R,E)-11-(3-(5-((S)-2-amino-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

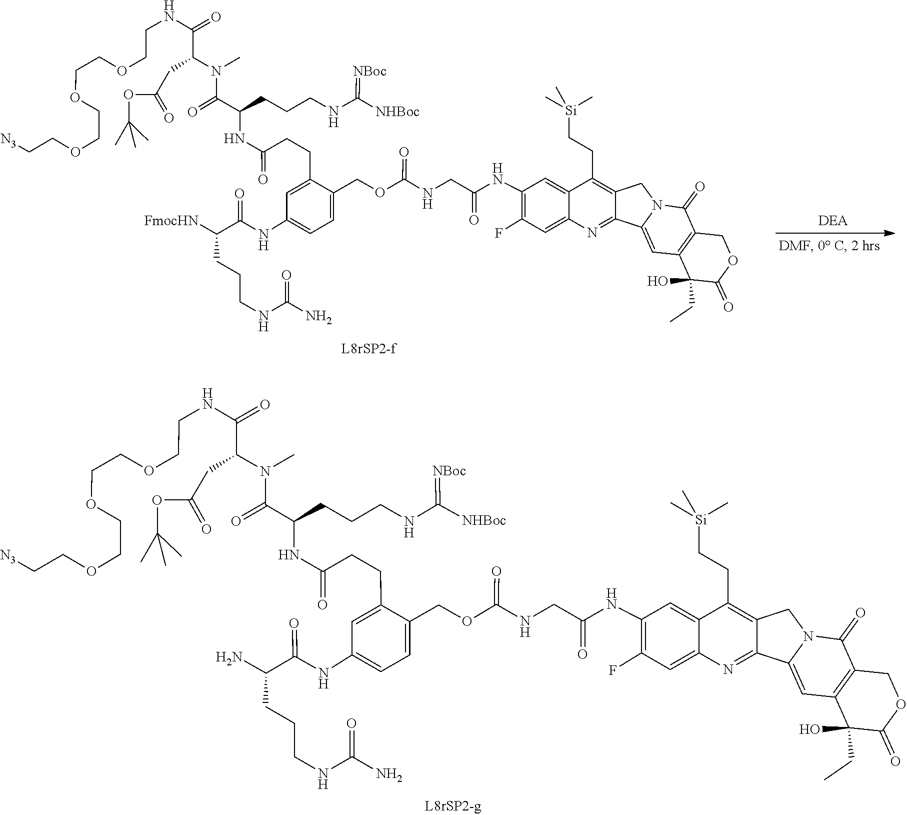

Intermediate L8rSP2-f (tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 742 mg, 0.39 mmol) was dissolved in DMF (CAS RN: 68-12-2; 4 mL) at 0° C. Then, diethylamine (CAS RN: 109-89-7; 0.41 mL, 3.95 mmol) was added to the reaction mixture, and the reaction mixture was left stirring at 0° C. for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-g (tert-butyl (11R,14R,E)-11-(3-(5-((S)-2-amino-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 370 mg, 0.22 mmol, 57.22% in yield) as a yellow solid.

LC-MS (m/z, C₇₇H₁₁₂FN₁₇O₂₁Si)=830.0[M/2+H]⁺.

### Intermediate L8rSP2-h: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

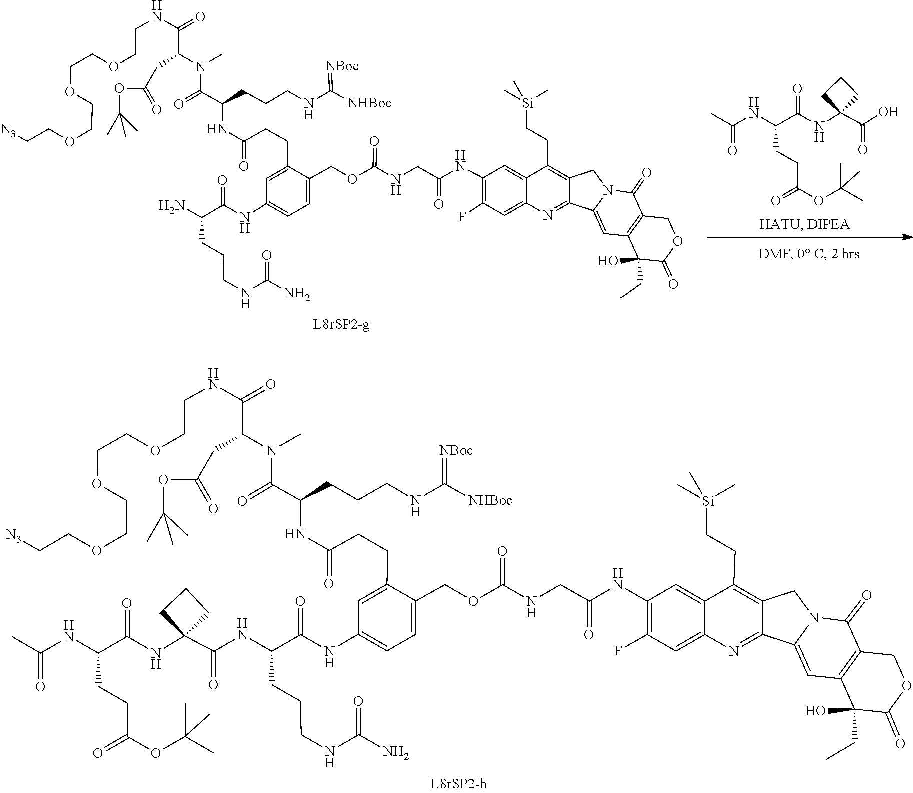

Intermediate HPH-L02 ((S)-1-(2-acetamido-5-(tert-butoxy)-5-oxopentanamido)cyclobutane-1-carboxylic acid; 77 mg, 0.22 mmol) was dissolved in DMF (CAS RN: 68-12-2; 3 ml) at 0° C. Then, DIPEA (CAS RN: 7087-68-5; 44 mg, 0.34 mmol) and HATU (CAS RN: 148893-10-1; 128 mg, 0.33 mmol) were added to the reaction mixture, and the reaction mixture was stirred at 0° C. for 2 hours before the addition of intermediate L8rSP2-g (tert-butyl (11R,14R,E)-11-(3-(5-((S)-2-amino-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 370 mg, 0.22 mmol). The reaction mixture was left stirring at 0° C. for additional 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-h (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 340 mg, 0.17 mmol, 77.93% in yield) as a white solid.

LC-MS (m/z, C₉₃H₁₃₆FN₁₉O₂₆Si)=992.4[M/2+H]⁺.

### Intermediate L8rSP2-i: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

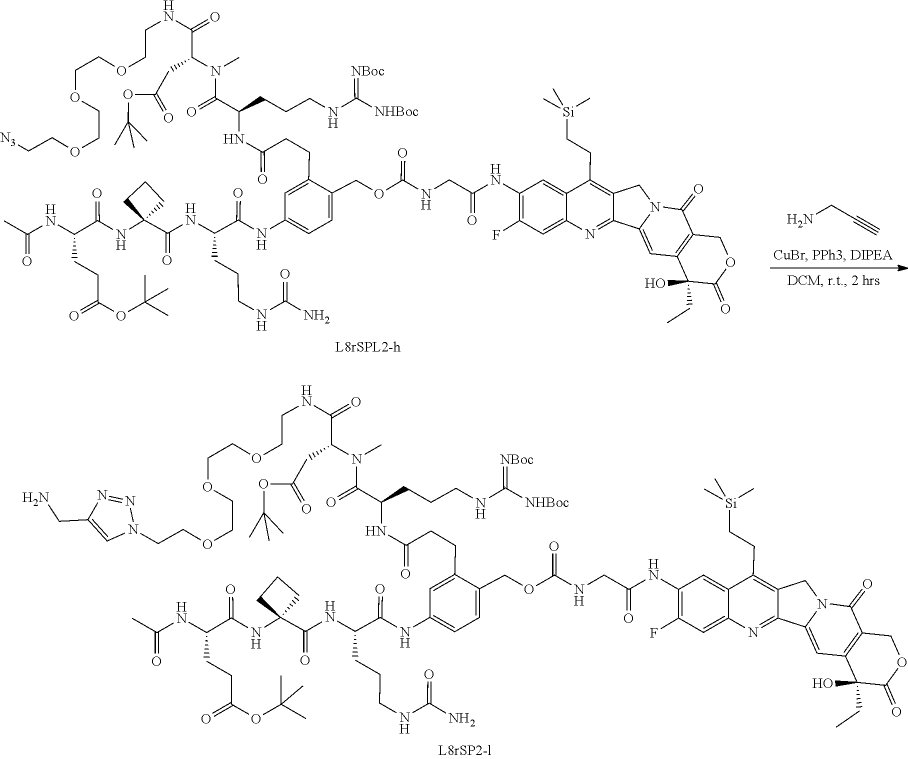

Intermediate L8rSP2-h (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 340 mg, 0.17 mmol) was dissolved in DCM (CAS RN: 75-09-2; 4 mL) at room temperature. Then, triphenylphosphine (CAS RN:603-35-0; 45 mg, 0.17 mmol), DIPEA (CAS RN: 7087-68-5; 23 mg, 0.18 mmol), propargylamine (CAS RN:2450-71-7; 10 mg, 0.18 mmol) and copper(I) bromide (CAS RN:7787-70-4; 25 mg, 0.17 mmol) were added to the reaction mixture, and the reaction mixture was stirred at room temperature for 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-i (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 130 mg, 0.06 mmol, 37.49% in yield) as a white solid.

LC-MS (m/z, C₉₆H₁₄₁FN₂₀O₂₆Si)=1020.0[M/2+H]⁺

### Intermediate L8rSP2-j: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate

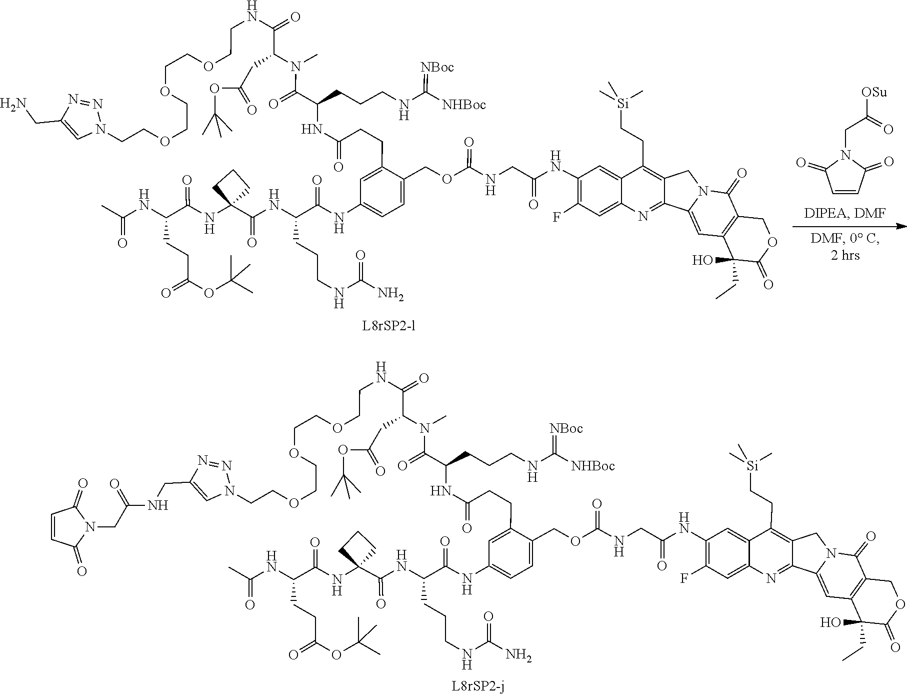

Intermediate L8rSP2-i (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 60 mg, 0.03 mmol) was dissolved in DMF (CAS RN: 68-12-2; 3 mL) at 0° C. Then, DIPEA (DIPEA; CAS RN: 7087-68-5, 8 mg, 0.06 mmol) and N-succinimidyl maleimidoacetate (CAS RN: 55750-61-3, 8 mg, 0.0317 mmol) were added to the reaction mixture, and the reaction mixture was further stirred at 0° C. for another 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rSP2-j (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 40 mg, 0.0184 mmol, 61.33% in yield) as a white solid.

LC-MS (m/z, C₁₀₂H₁₄₄FN₂₁O₂₉Si)=1088.3[M/2+H]⁺.

### Linker-payload L8rSP2: (R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

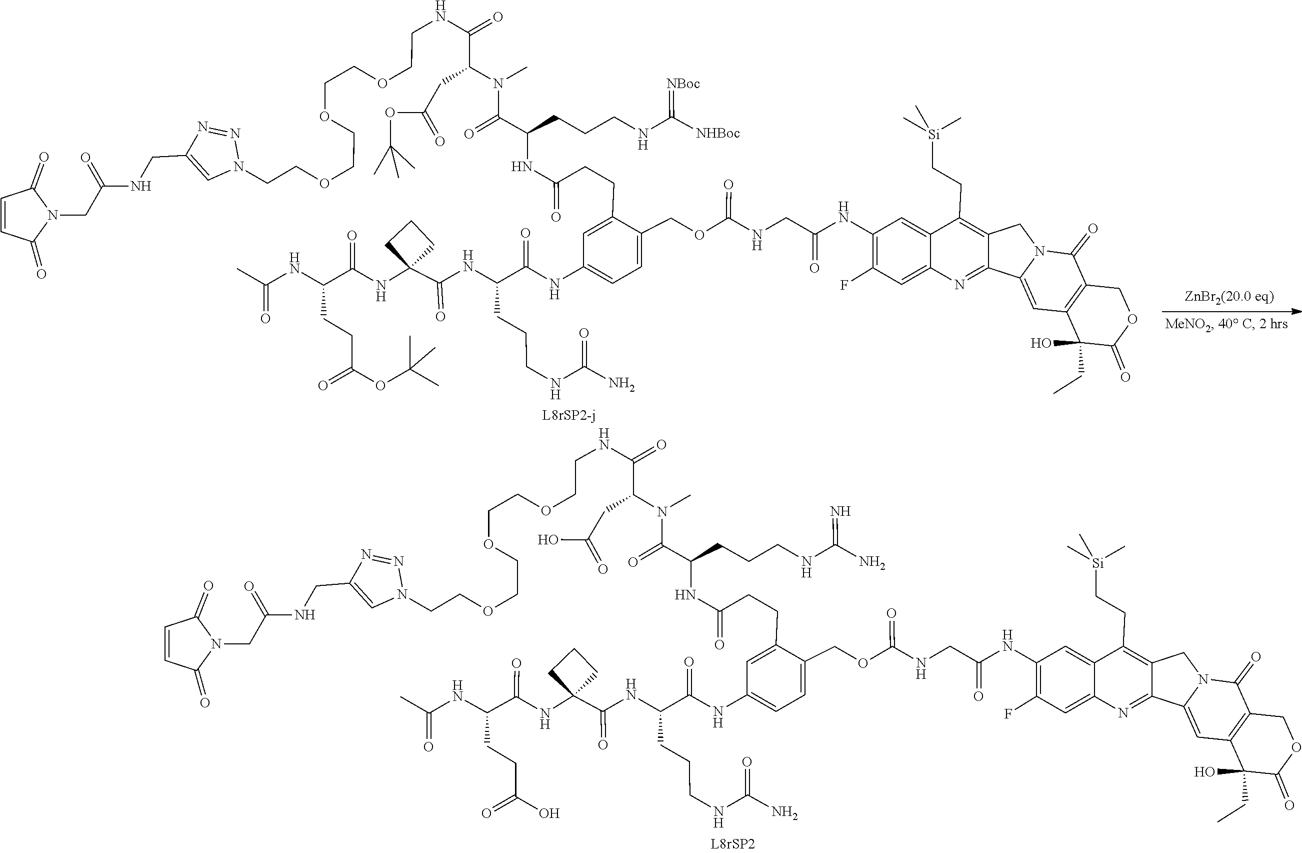

Intermediate L8rSP2-j (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 40 mg, 0.0184 mmol) was dissolved in nitromethane (CAS RN: 75-52-5; 3 ml) at 40° C. Then, zinc bromide (CAS RN: 7699-45-8, 83 mg, 0.364 mmol) was added to the reaction mixture, and the reaction mixture was stirred for 2 hours at 40° C. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L8rSP2 ((R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 8.83 mg, 0.0047 mmol, 25.76% in yield after purification) as a white solid.

LC-MS (m/z, C₈₄H₁₁₂FN₂₁O₂₅Si)=932.2[M/2+H]⁺.

### Example 14: Linker-Payload L8rTP2

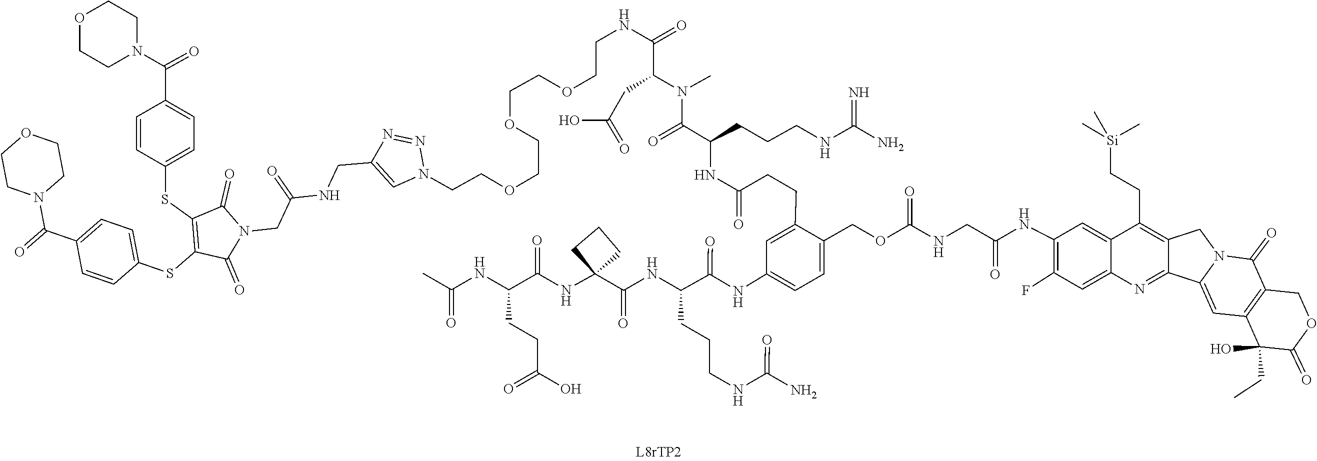

### Intermediate L8rTP2-a: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

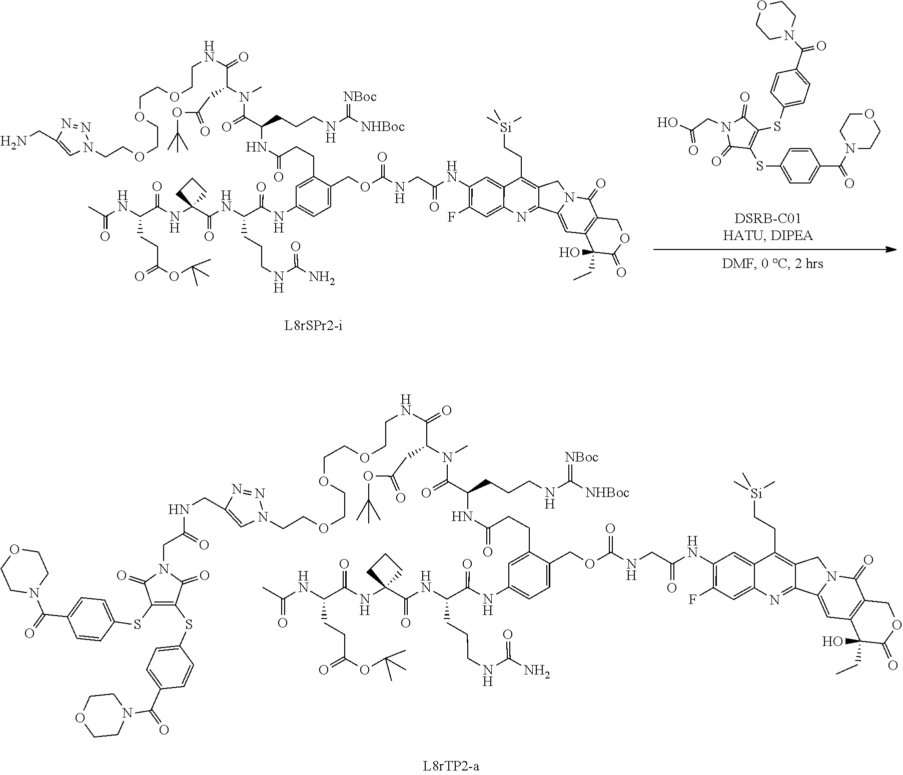

Intermediate L8rSP2-i (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 60 mg, 0.03 mmol) was dissolved in DMF (CAS RN: 68-12-2; 2 mL) at 0° C. Then, DIPEA (CAS RN: 7087-68-5, 12 mg, 0.09 mmol), HATU (CAS RN: 148893-10-1; 36 mg, 0.09 mmol) and intermediate DSRB-C01 (2-(3,4-bis((4-(morpholine-4-carbonyl)phenyl)thio)-2,5-dioxo-2,5-dihydro-1H-pyrrol-1-yl)acetic acid; 40 mg, 0.067 mmol) were added to the reaction mixture, and the reaction mixture was further stirred at 0° C. for another 2 hours. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired intermediate L8rTP2-a (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 45 mg, 0.0172 mmol, 57.31% in yield) as a yellow solid.

LC-MS (m/z, C₉₈H₁₂₃FN₁₄O₂₇S₂)=1309.6[M/2+H]⁺.

### Linker-payload L8rTP2: (R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

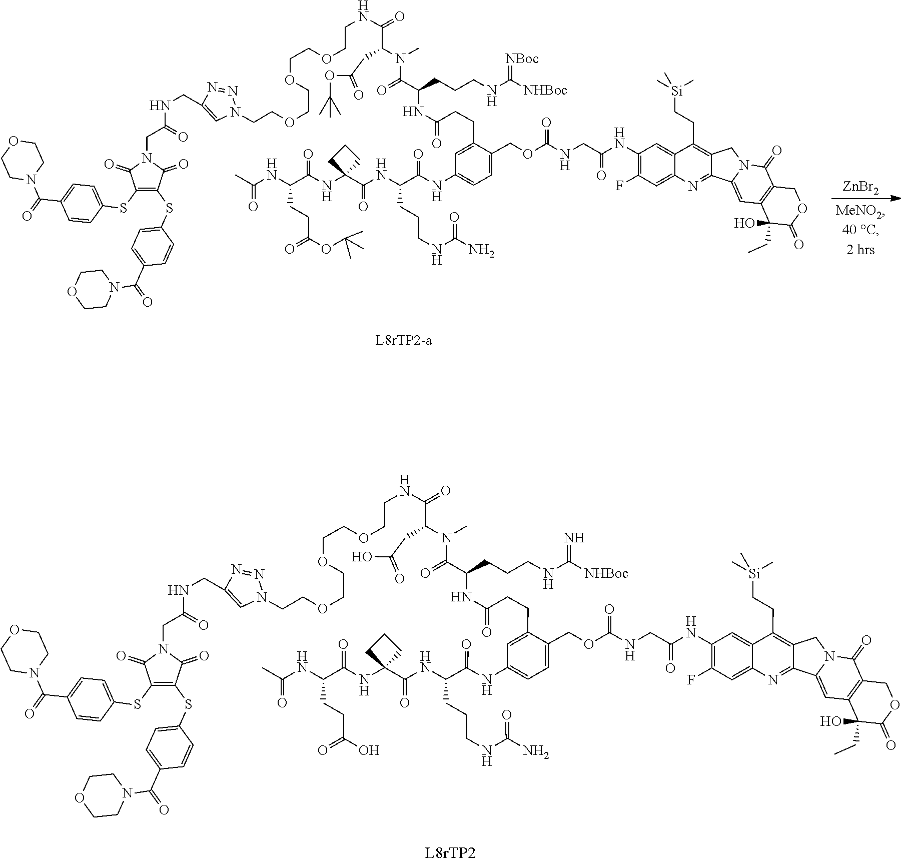

Intermediate L8rTP2-a (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 45 mg, 0.0172 mmol) was dissolved in nitromethane (CAS RN: 75-52-5; 3 ml) at 40° C. Then, zinc bromide (CAS RN: 7699-45-8, 120 mg, 0.53 mmol) was added to the reaction mixture, and the reaction mixture was stirred for 2 hours at 40° C. LC-MS was used to confirm the full consumption of starting material in the reaction mixture. The organic solvent in the reaction mixture was reduced in vacuo to afford the reaction residue. The residue was further purified by preparative reverse-phase liquid chromatography (Column: Welch-Ultimate-C18-10 μm-21.2*150 mm, mobile phase A: 0.1% TFA in H₂O; mobile phase B: ACN) to recover the desired linker-payload L8rTP2 ((R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-3,14-dioxo-11-(2-(trimethylsilyl)ethyl)-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 12 mg, 0.0052 mmol, 30.27% in yield after purification) as a yellow solid.

LC-MS (m/z, C₁₀₆H₁₃₄FN₂₃O₂₉S₂Si)=1153.4[M/2+H]⁺.

### Comparative Example 1: Synthesis of Control Payloads

### Payload Pr1

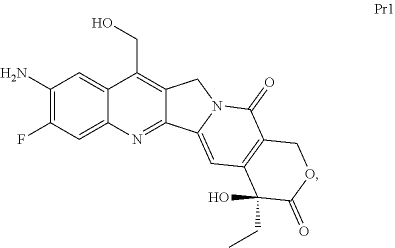 [SMILES: CC[C@@]1(O)C(=O)OCc2c1cc1n(c2=O)Cc2c-1nc1cc(F)c(N)cc1c2CO]

the synthesis procedures for Payload Pr1 followed the description of compound 141 in WO2022246576.

### Payload Pr2

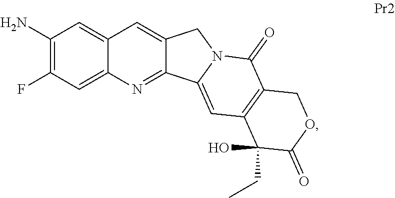 [SMILES: CC[C@@]1(O)C(=O)OCc2c1cc1n(c2=O)Cc2cc3cc(N)c(F)cc3nc2-1]

the synthesis procedures for Payload Pr2 followed the description of compound 140 in WO2022246576.

### Comparative Example 2: Synthesis of Linker-Payload

### Linker-Payload L8rMPr1

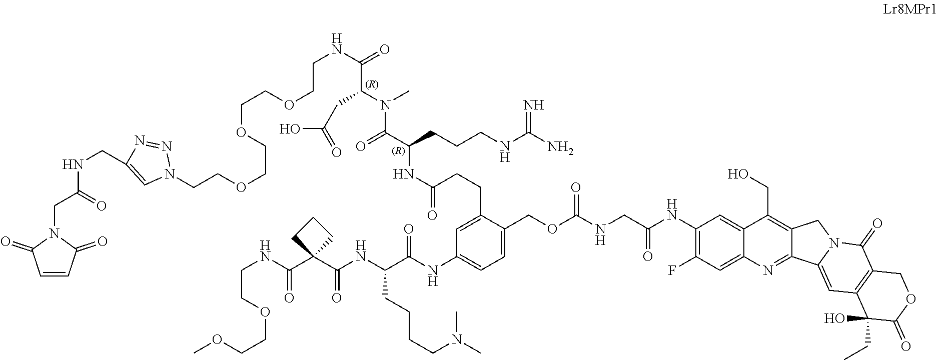

### Intermediate L8rMPr1-a: tert-butyl (S)-(2-((4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)carbamate

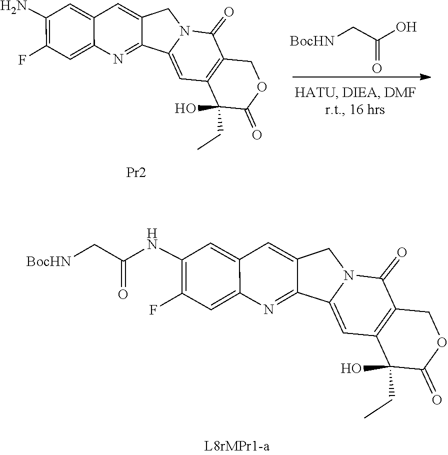 [SMILES: CC[C@@]1(O)C(=O)OCc2c1cc1n(c2=O)Cc2cc3cc(N)c(F)cc3nc2-1 ; CC(C)(C)OC(=O)NCC(=O)O ; "Pr2" ; CC[C@@]1(O)C(=O)OCc2c1cc1n(c2=O)Cc2cc3cc(NC(=O)CNC(=O)OC(C)(C)C)c(F)cc3nc2-1]

Payload Pr2 ((S)-9-amino-4-ethyl-8-fluoro-4-hydroxy-1,12-dihydro-14H-pyrano[3′,4′:6,7]indolizino[1,2-b]quinoline-3,14(4H)-dione; 36 g, 94.40 mmol) was dissolved in DMF (CAS RN: 68-12-2; 360 mL) at room temperature. Then, HATU (CAS RN: 148893-10-1, 179.47 g, 471.99 mmol), DIPEA (CAS RN: 7087-68-5, 82.21 mL, 471.99 mmol) and Boc-glycine (CAS RN: 4530-20-5, 74.42 g, 424.79 mmol) were added to the reaction mixture, and the reaction mixture was left stirring at room temperature for additional 16 hours. LC-MS was used to confirm the full consumption of starting material in the reaction mixture. Icy water (300 ml) was poured into the reaction mixture to quench the reaction, and the mixture was stirred for 15 minutes. The solid in the reaction mixture was collected by filtration. And the dried solid was purified by silica chromatography (DCM:methanol=20:1) to afford the desirable intermediate L8rMPr1-a (tert-butyl (S)-(2-((4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)carbamate; 25.00 g, 46.42 mmol, 49.18% in yield) as a yellow solid.

LC-MS (m/z, C₂₇H₂₇FN₄O₇)=539.4 [M+H]⁺.

¹H NMR (400 MHz, DMSO-d₆) δ 10.10 (s, 1H), 8.77 (d, 1H), 8.65 (s, 1H), 8.00 (d, 1H), 7.30 (s, 1H), 7.21-7.09 (m, 1H), 6.55 (s, 1H), 5.44 (s, 2H), 5.21 (s, 2H), 3.93 (d, 2H), 1.93-1.84 (m, 2H), 1.43 (s, 9H), 0.90-0.86 (m, 3H).

### Intermediate L8rMPr1-b: tert-butyl (S)-(2-((4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamate

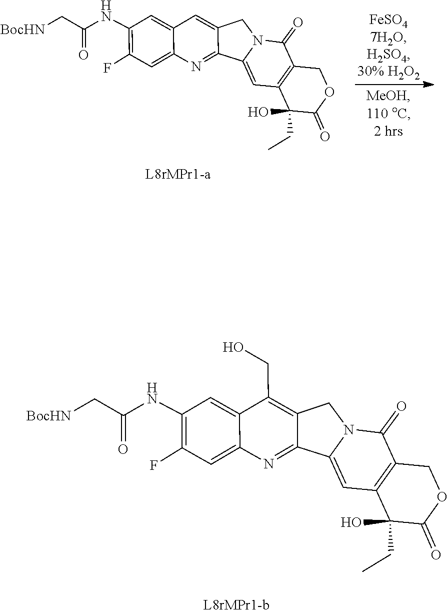 [SMILES: CC[C@@]1(O)C(=O)OCc2c1cc1n(c2=O)Cc2cc3cc(NC(=O)CNC(=O)OC(C)(C)C)c(F)cc3nc2-1 ; CC[C@@]1(O)C(=O)OCc2c1cc1n(c2=O)Cc2c-1nc1cc(F)c(NC(=O)CNC(=O)OC(C)(C)C)cc1c2CO]

Intermediate L8rMPr-a (tert-butyl (S)-(2-((4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)carbamate; 10.20 g, 18.94 mmol) was dissolved in anhydrous methanol (CAS RN: 67-56-1; 500 mL) at room temperature. Then, the reaction mixture was added with sulfuric acid (CAS RN: 7664-93-9; 1.02 mL, 1.33 mmol), ferrous sulphate heptahydrate (CAS RN: 7782-63-0; 5.27 g, 18.94 mmol) and 30% hydrogen peroxide solution (CAS RN: 7722-84-1; 0.64 g, 5.68 mmol, 30% in water). The reaction mixture was heated to 110° C. and stirred for another 2 hours at this temperature. Icy-cold water (300 mL) was poured into the reaction mixture to quench the reaction. The yellow solid in the reaction mixture was collected by filtration as the desirable intermediate L8rMPr1-b (tert-butyl (S)-(2-((4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamate; 4.5 g, 7.91 mmol, 41.79% in yield).

LC-MS (m/z, C₂₈H₂₉FN₄O₈)=569.2 [M+H]⁺.

¹H NMR (400 MHz, DMSO-d₆) δ 10.16 (s, 1H), 8.86 (d, 1H), 8.02 (d, 1H), 7.30 (s, 1H), 7.19-7.16 (m, 1H), 6.50 (s, 1H), 5.41 (s, 2H), 5.39 (s, 2H), 5.14 (s, 2H), 3.89 (d, 2H), 1.92-1.81 (m, 2H), 1.40 (s, 9H), 0.92-0.84 (m, 3H).

### Intermediate L8rMPr1-c: (S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide

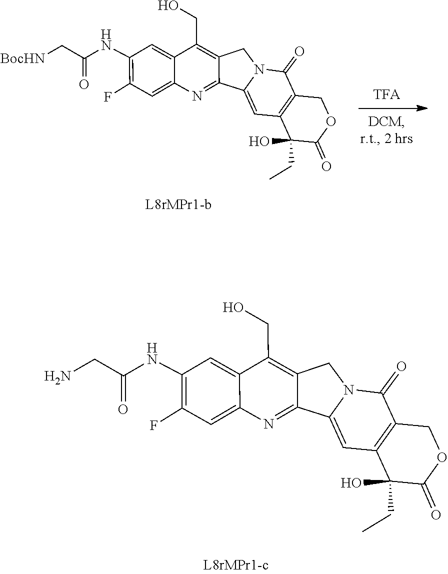 [SMILES: CC[C@@]1(O)C(=O)OCc2c1cc1n(c2=O)Cc2c-1nc1cc(F)c(NC(=O)CNC(=O)OC(C)(C)C)cc1c2CO ; CC[C@@]1(O)C(=O)OCc2c1cc1n(c2=O)Cc2c-1nc1cc(F)c(NC(=O)CN)cc1c2CO]

Intermediate L8rMPr1-b (tert-butyl (S)-(2-((4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamate; 4.5 g, 7.91 mmol) was dissolved in DCM (CAS: 75-09-2, 10 mL) at room temperature. Then TFA (CAS. RN: 76-05-1, 3.3 mL) was added to the reaction mixture and the reaction mixture was left stirring for another 2 hours. LC-MS was used to verify the full consumption of starting materials in the reaction mixture. The excess organic solvent and TFA in the reaction mixture were reduced in vacuo to afford the reaction residue. The residue was purified by preparative reverse-phase liquid chromatography (acetonitrile/water (formic acid) system) (water (0.1% TFA)/ACN=80%/20%), UV=254 nm, Puninglech-Pntulips-C18-10-30*250 mm, 25° C., 20 mL/min) to afford the desirable intermediate L8rMPr1-c ((S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide; 3.60 g, 7.68 mmol, 97.10% in yield) as a light yellow solid.

LC-MS (m/z, C₂₃H₂₁FN₄O₆)=469.2[M+H]⁺.

¹H NMR (400 MHz, DMSO-d₆) δ 10.73 (s, 1H), 8.91 (d, 1H), 8.15 (s, 2H), 8.11 (d, 1H), 7.31 (s, 1H), 6.50 (s, 1H), 5.85 (s, 1H), 5.43 (s, 2H), 5.41 (s, 2H), 5.16 (s, 2H), 3.98 (s, 2H), 1.93-1.81 (m, 2H), 0.90-0.86 (m, 3H).

### Intermediate L8rMPr1-d: tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate

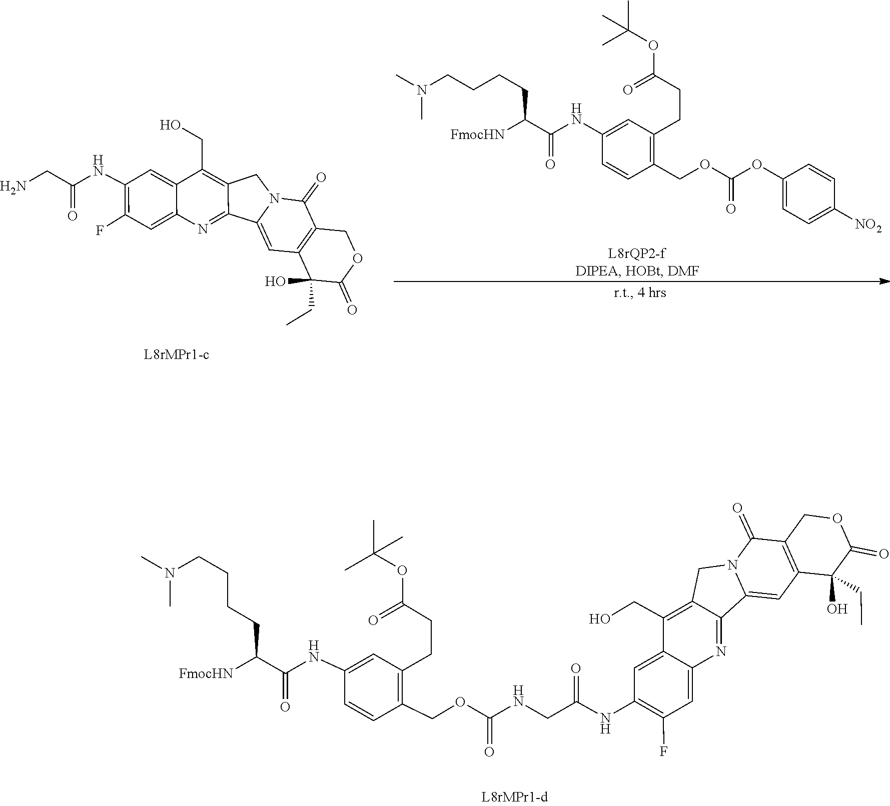

The synthesis procedures of intermediate L8rMPr1-d referred to the synthesis procedures of intermediate L8rQP2-g. Using intermediate L8rMPr1-c ((S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide; 1.20 g, 2.56 mmol) and intermediate L8rQP2-f (tert-butyl (S)-3-(5-(2-((((9H-fluoren-9-yl)methoxy)carbonyl)amino)-6-(dimethylamino)hexanamido)-2-((((4-nitrophenoxy)carbonyl)oxy)methyl)phenyl)propanoate; 1.67 g, 2.23 mmol) as the starting material, the desirable intermediate L8rMPr1-d (tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanoate; 1.30 g, 1.16 mmol, 45.14% in yield) was obtained as a light yellow solid.

LC-MS (m/z, C61H66FN7O13)=1124.4 [M+H]⁺.

### Intermediate L8rMPr1-e: 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid

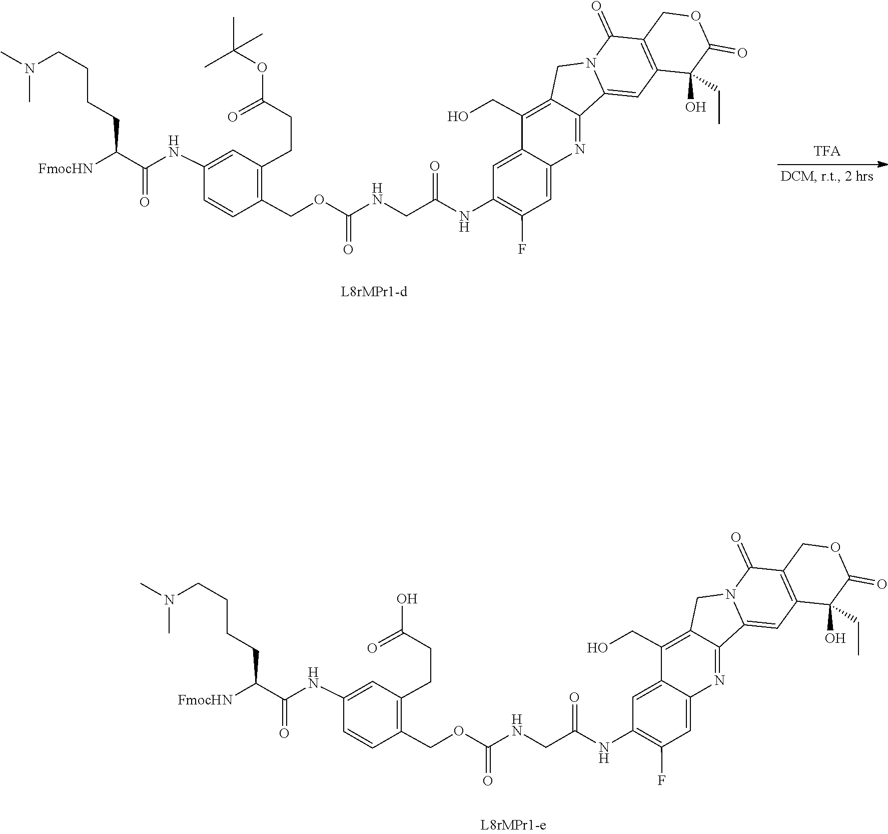

The synthesis procedures of intermediate L8rMPr1-e referred to the synthesis procedures of intermediate L8rQP2-h. Using intermediate L8rMPr1-d (tert-butyl 3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 1.30 g, 1.16 mmol) as the starting material, the desirable intermediate L8rMPr1-e (3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanoic acid; 0.80 g, 0.75 mmol, 64.77% in yield) was recovered as a light yellow solid.

LC-MS (m/z, C57H58FN7O13)=1068.4 [M+H]⁺.

### Intermediate L8rMPr1-f: tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

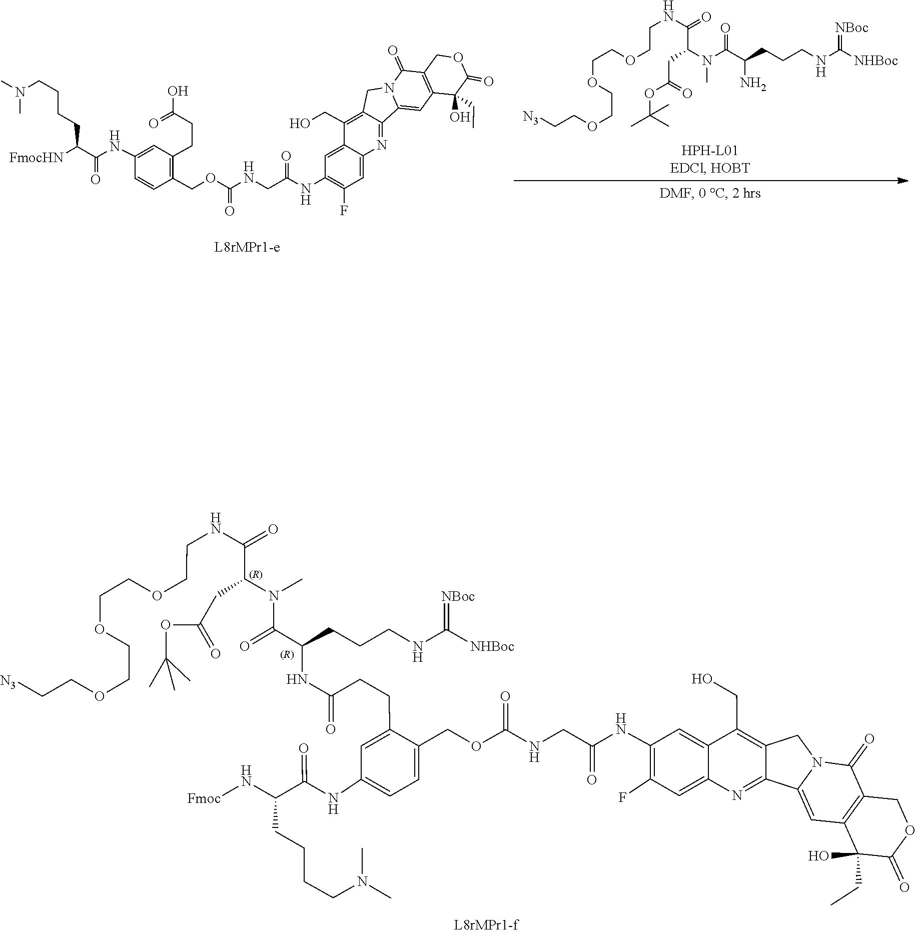

The synthesis procedures of intermediate L8rMPr1-f referred to the synthesis procedures of intermediate L8rQP2-i. Using intermediate L8rMPr1-e (3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 760 mg, 0.71 mmol) and intermediate HPH-L01 (tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 540 mg, 0.71 mmol) as the starting material, the desirable intermediate L8rMPr1-f (tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 570 mg, 0.315 mmol, 44.37% in yield) was recovered as a yellow solid.

LC-MS (m/z, C₉₀H₁₁₇FN₁₆O₂₃)=905.6 [M/2+H]⁺.

### Intermediate L8rMPr1-g: tert-butyl (11R,14R,E)-11-(3-(5-((S)-2-amino-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

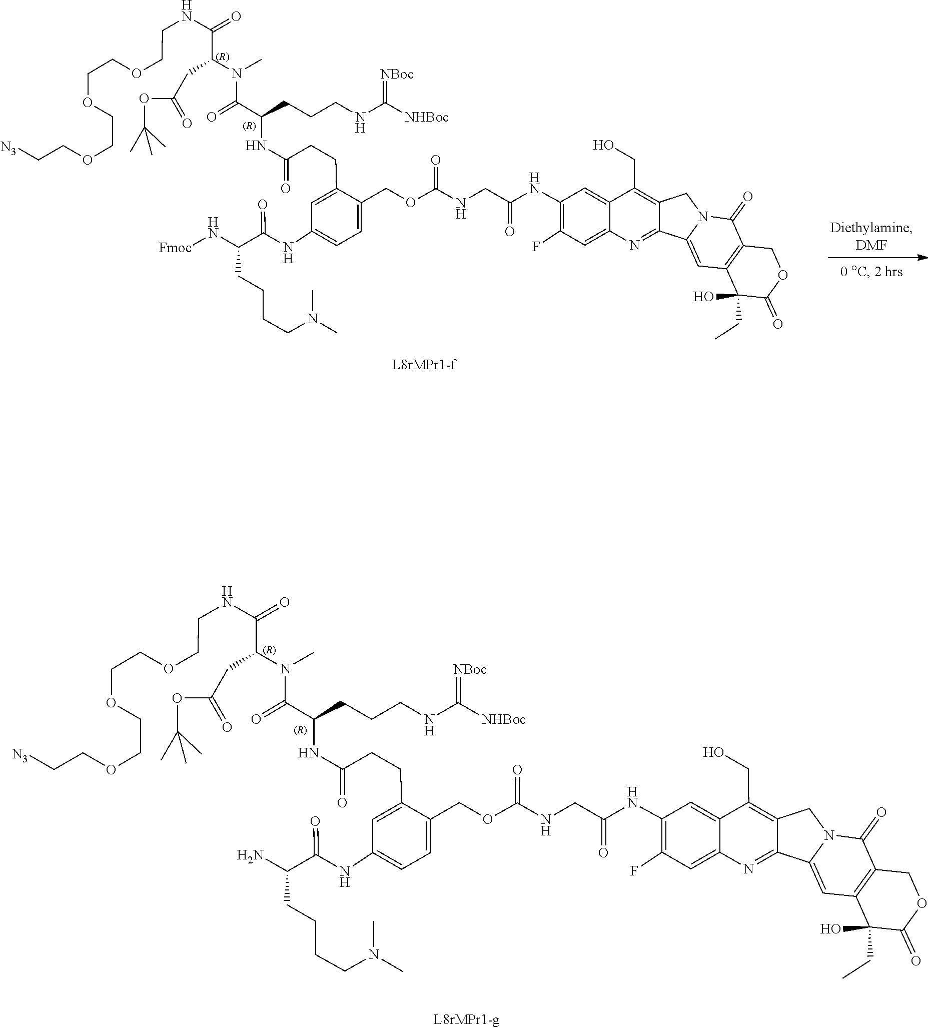

The synthesis procedures of intermediate L8rMPr1-g referred to the synthesis procedures of intermediate L8rQP2-j. Using intermediate L8rMPr1-f (tert-butyl (11R,14R,E)-11-(3-(5-((S)-2-((((9H-fluoren-9-yl)methoxy)carbonyl)amino)-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 570 mg, 0.26 mmol) as the starting material, the desirable intermediate L8rMPr1-g (tert-butyl (11R,14R,E)-11-(3-(5-((S)-2-amino-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 330 mg, 0.208 mmol, 80.0% in yield) was recovered as a yellow solid.

LC-MS (m/z, C₇₅H₁₀₇FN₁₆O₂₁)=794.6 [M/2+H]⁺.

### Intermediate L8rMPr1-h: tert-butyl (11R,14R,E)-14-((2-(2-(2-(2-azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

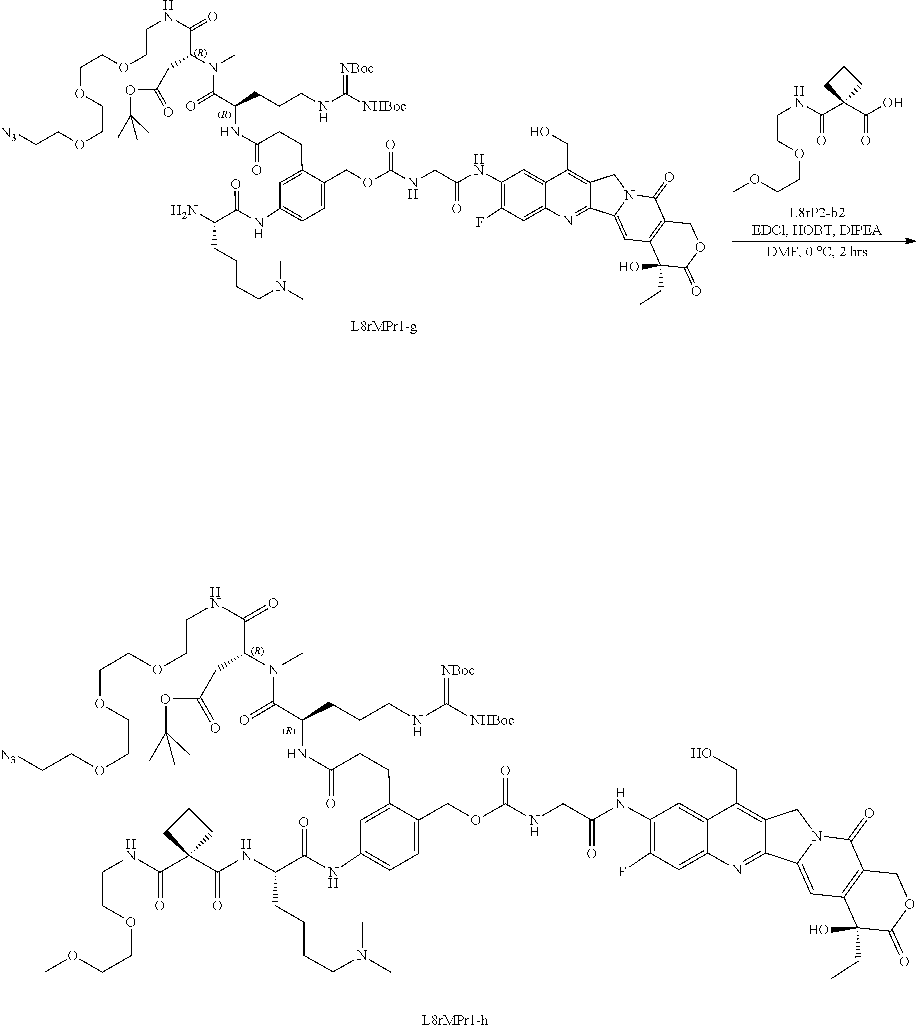

The synthesis procedures of intermediate L8rMPr1-h referred to the synthesis procedures of intermediate L8rQP2-k. Using intermediate L8rMPr1-g (tert-butyl (11R,14R,E)-11-(3-(5-((S)-2-amino-6-(dimethylamino)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 330 mg, 0.21 mmol) and intermediate L8rP2-b2 (1-((2-(2-methoxyethoxy)ethyl)carbamoyl)cyclobutane-1-carboxylic acid; 102 mg, 0.42 mmol) as the starting material, the desirable intermediate L8rMPr1-h (tert-butyl (11R,14R,E)-14-((2-(2-(2-(2-azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 180 mg, 0.0992 mmol, 47.25% in yield) was recovered as a yellow solid.

LC-MS (m/z, C₈₆H₁₂₄FN₁₇O₂₅)=908.0 [M/2+H]⁺.

### Intermediate L8rMPr1-i: tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

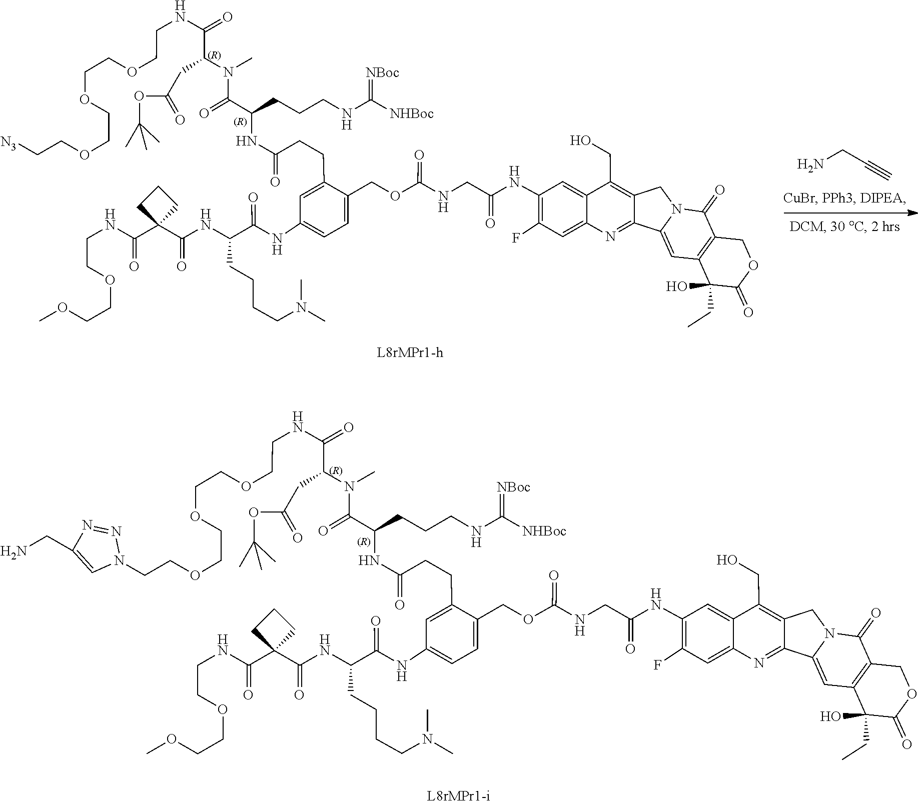

The synthesis procedures of intermediate L8rMPr1-i referred to the synthesis procedures of intermediate L8rQP2-1. Using intermediate L8rMPr1-h (tert-butyl (11R,14R,E)-14-((2-(2-(2-(2-azidoethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 180 mg, 0.10 mmol) as the starting material, the desirable intermediate L8rMPr1-i (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 140 mg, 0.075 mmol, 75.78% in yield) was recovered as a yellow solid.

LC-MS (m/z, C₈₉H₁₂₉FN₁₈O₂₅)=935.6 [M/2+H]⁺.

### Intermediate L8rMPr1-j: tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate

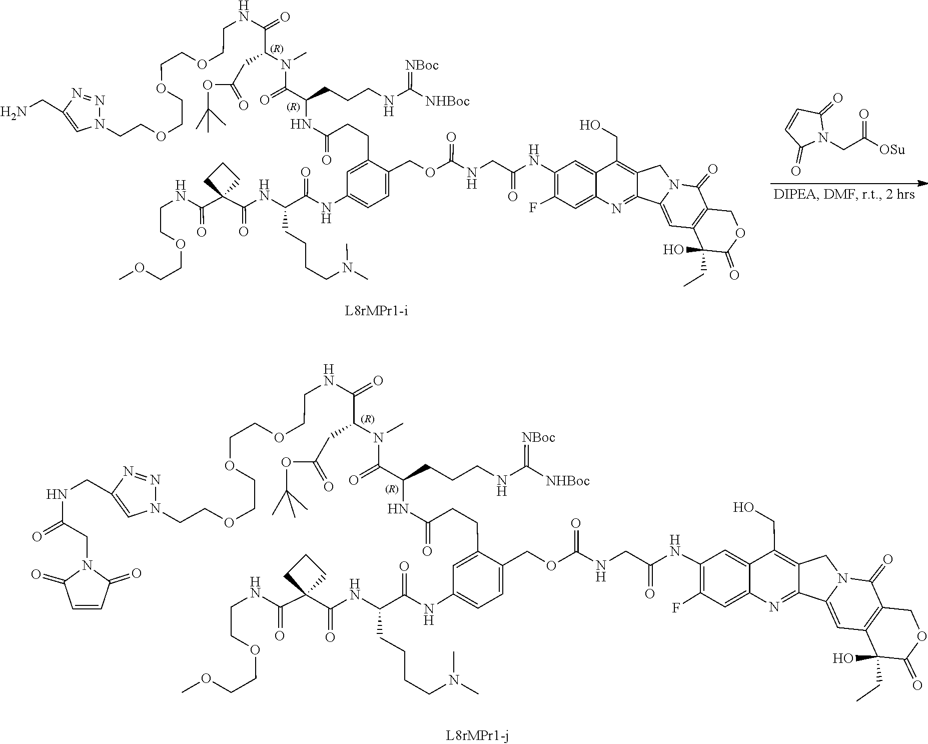

The synthesis procedures of intermediate L8rMPr1-j referred to the synthesis procedures of intermediate L8rQP2-m. Using intermediate L8rMPr1-i (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 80 mg, 0.0428 mmol) as the starting material, the desirable intermediate L8rMPr1-j (tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 70 mg, 0.0349 mmol, 81.54% in yield) was recovered as a yellow solid.

LC-MS (m/z, C₉₅H₁₃₂FN₁₉O₂₈)=1004.2 [M/2+H]⁺.

### Linker-payload L8rMP1: (R)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

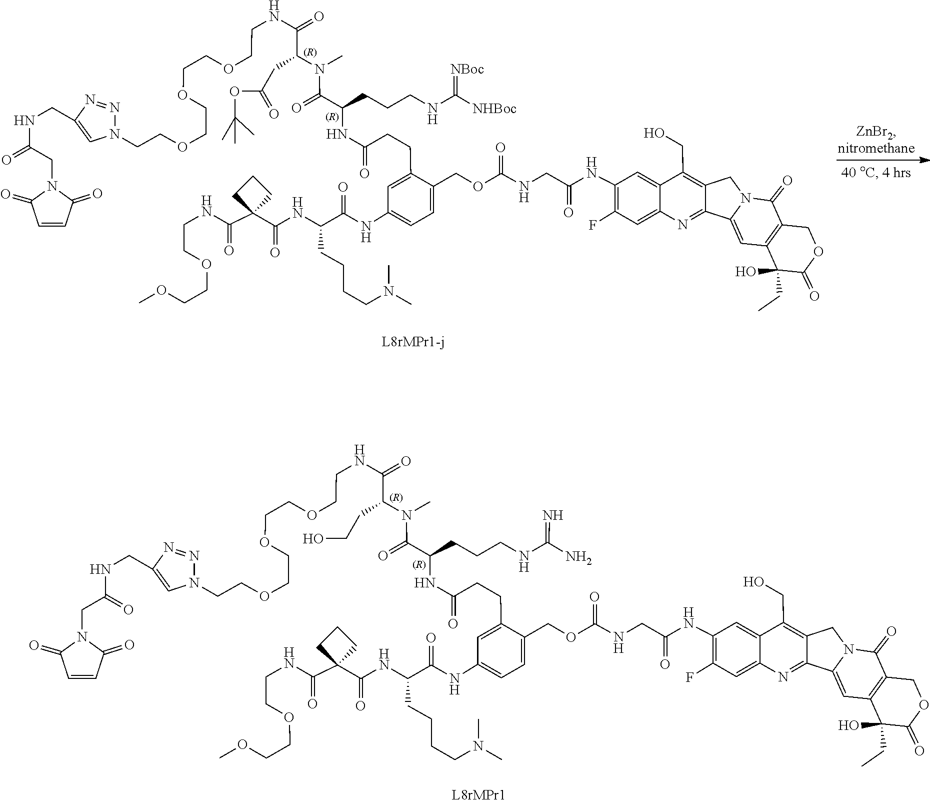

The synthesis procedures of linker-payload L8rMPr1 referred to the synthesis procedures of linker-payload L8rQP2. Using intermediate L8rMPr1-j (tert-butyl (11R,14R,E)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 70 mg, 0.0349 mmol) as the starting material, the desirable intermediate L8rMPr1 ((R)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 7.83 mg, 0.00447 mmol, 12.81% in yield after purification) was recovered as a yellow solid.

LC-MS (m/z, C₈₁H₁₀₈FN₁₉O₂₄)=1751.8 [M+H]⁺.

### Linker-Payload L8rNPr1

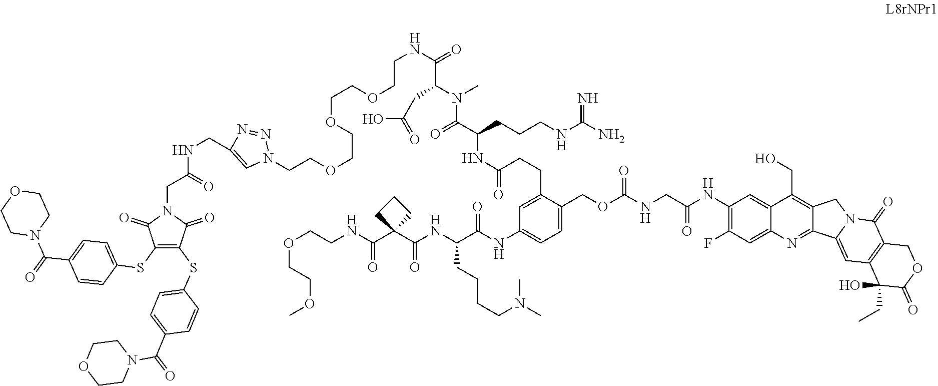

Intermediate L8rNPr1-a: tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate

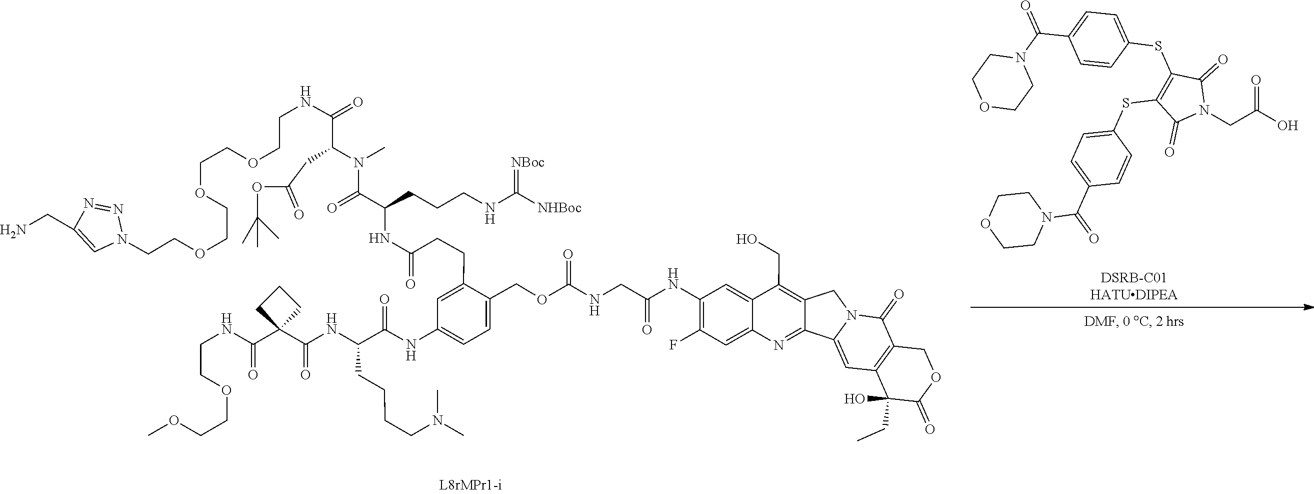

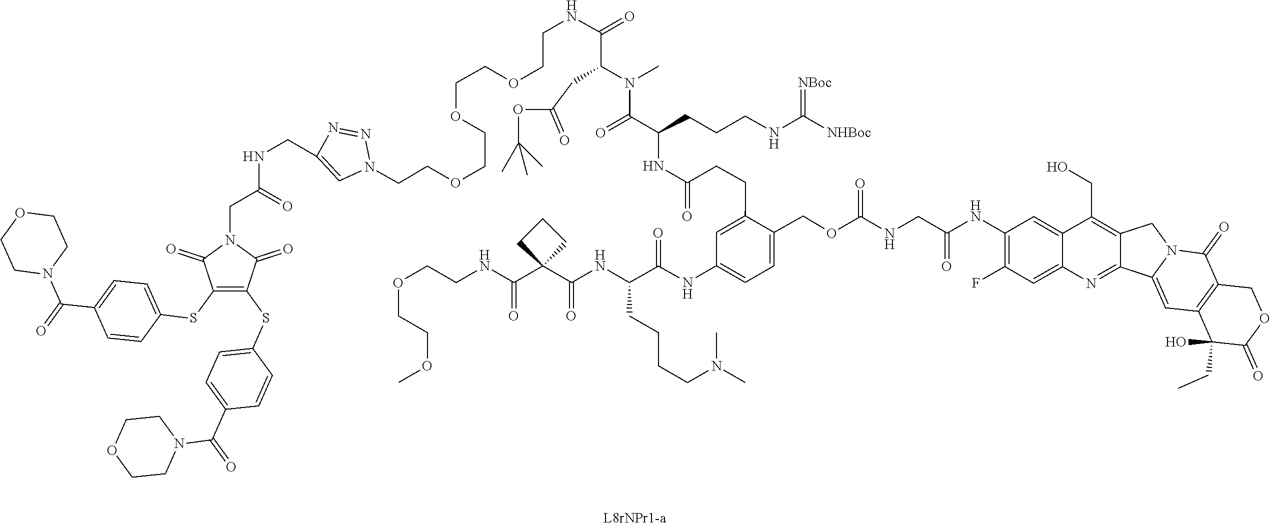

The synthesis procedures of intermediate L8rNPr1-a referred to the synthesis procedures of intermediate L8rRP2-a. Using intermediate L8rMPr1-i (tert-butyl (11R,14R,E)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 80 mg, 0.0428 mmol) as the starting material, the desirable intermediate L8rNPr1-a (tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 60 mg, 0.0245 mmol, 57.24% in yield) was recovered as a yellow solid.

LC-MS (m/z, C₁₁₇H₁₅₄FN₂₁O₃₂S₂)=1225.3 [M/2+H]⁺.

### Linker-payload L8rNPr1: (R)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-13-oxo-3,6,9-trioxa-12-azahexadecan-16-oic acid

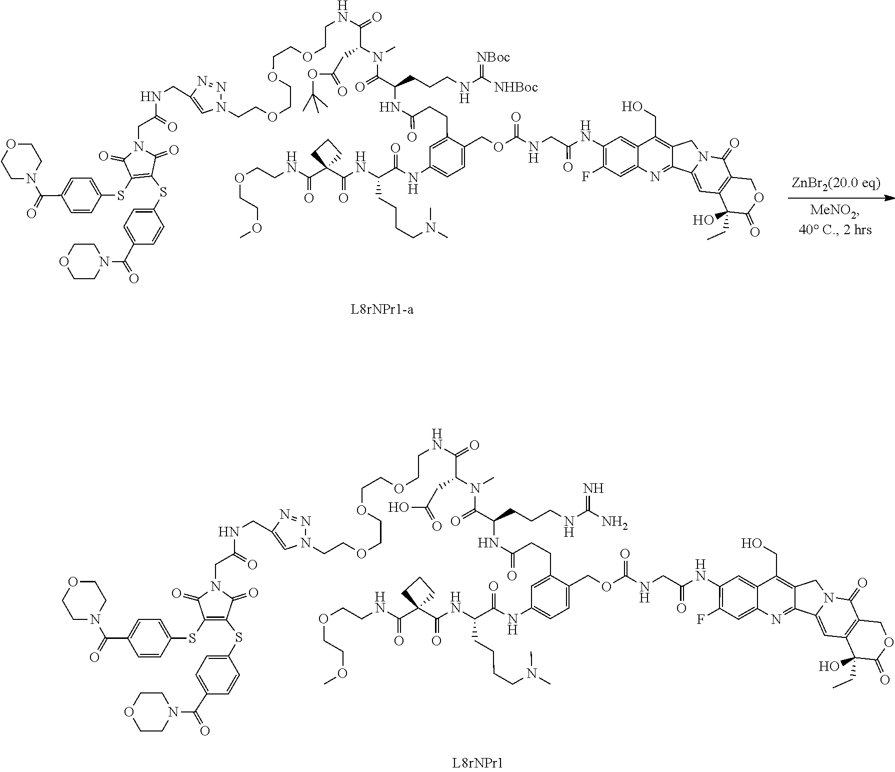

The synthesis procedures of linker-payload L8rNPr1 referred to the synthesis procedures of linker-payload L8rRP2. Using intermediate L8rNPr1-a (tert-butyl (11R,14R,E)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-11-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-2,2,13-trimethyl-4,12-dioxo-3-oxa-5,7,13-triazahexadec-5-en-16-oate; 60 mg, 0.0245 mmol) as the starting material, the desirable linker-payload L8rNPr1 ((R)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-14-((R)-2-(3-(5-((S)-6-(dimethylamino)-2-(1-((2-(2-methoxyethoxy)ethyl)carbamoyl)cyclobutane-1-carboxamido)hexanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-13-oxo-3,6,9-trioxa-12-azahexadecan-16-oic acid; 6.25 mg, 0.00285 mmol, 11.63% in yield after purification) was recovered as a yellow solid.

LC-MS (m/z, C₁₀₃H₁₃₀FN₂₁O₂₈S₂)=1097.4 [M/2+H]⁺.

### Linker-Payload L8rOPr1

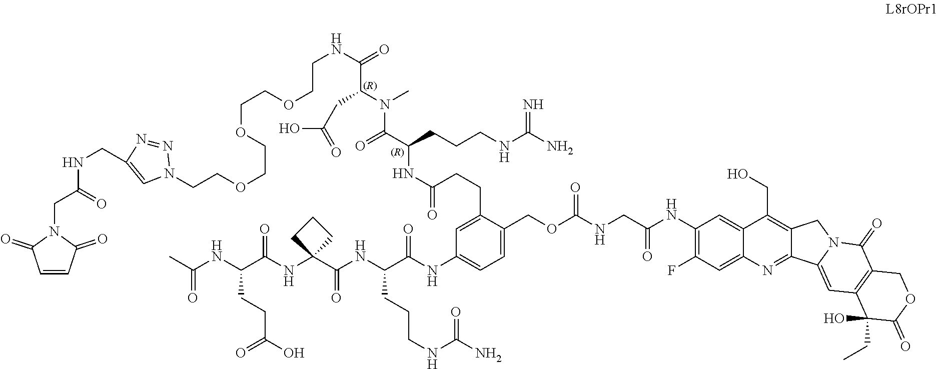

### Intermediate L8rOPr1-a: tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate

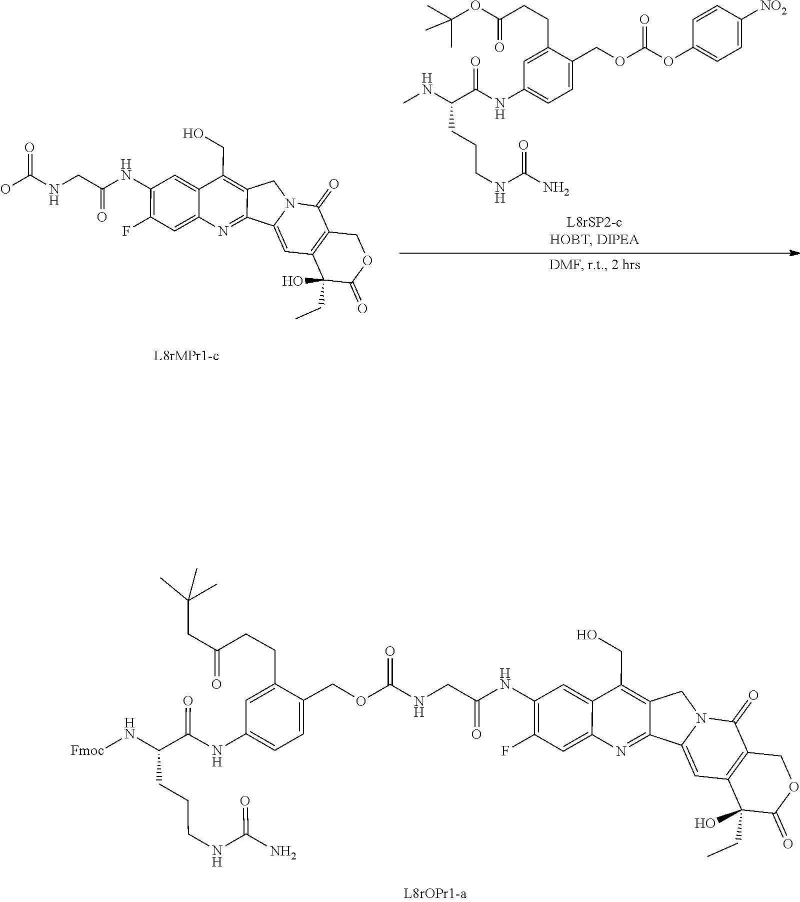

The synthesis procedures of intermediate L8rOPr1-a referred to the synthesis procedures of intermediate L8rSP2-d. Using intermediate L8rMPr1-c ((S)-2-amino-N-(4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)acetamide; 2 g, 4.27 mmol) and intermediate L8rSP2-c (tert-butyl (S)-3-(5-(2-((((9H-fluoren-9-yl)methoxy)carbonyl)amino)-5-ureidopentanamido)-2-((((4-nitrophenoxy)carbonyl)oxy)methyl)phenyl)propanoate; 2.88 g, 3.84 mmol) as the starting material, the desirable intermediate L8rOPr1-a (tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 1.50 g, 1.33 mmol, 31.19% in yield) was obtained as a light yellow solid.

LC-MS (m/z, C59H61FN8O14)=1126.4 [M+H]⁺.

### Intermediate L8rOPr1-b: 3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid

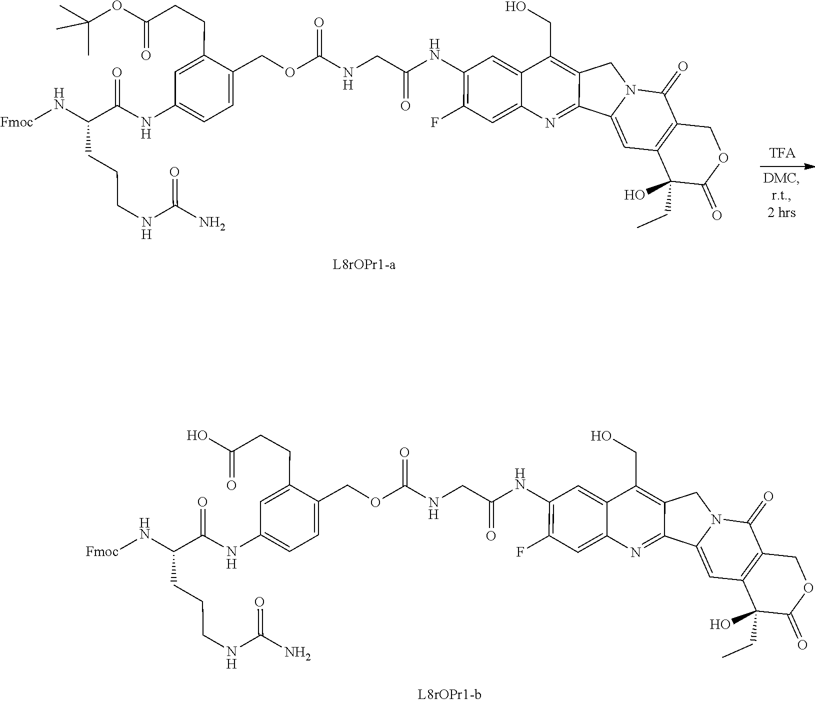

The synthesis procedures of intermediate L8rOPr1-b referred to the synthesis procedures of intermediate L8rSP2-e. Using intermediate L8rOPr1-a (tert-butyl 3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoate; 1.60 g, 1.42 mmol) as the starting material, the desirable intermediate L8rOPr1-b (3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanoic acid; 1.50 g, 1.40 mmol, 98.59% in yield) was obtained as a yellow solid.

LC-MS (m/z, C55H53FN8O14)=1069.4 [M+H]⁺.

### Intermediate L8rOPr1-c: tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

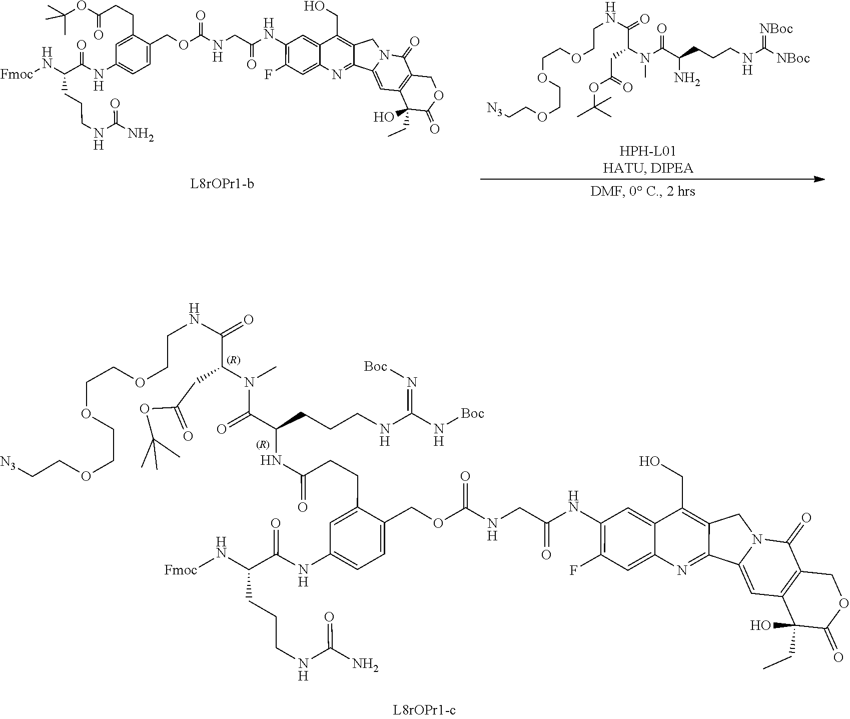

The synthesis procedures of intermediate L8rOPr1-c referred to the synthesis procedures of intermediate L8rSP2-f Using intermediate L8rOPr1-b (3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanoic acid; 2.74 g, 2.56 mmol) and intermediate HPH-L01 (tert-butyl (11R,14R,Z)-11-amino-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 1.50 g, 1.97 mmol) as the starting material, the desirable intermediate L8rOPr1-c (tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 0.80 g, 0.44 mmol, 22.43% in yield) was obtained as a yellow solid.

LC-MS (m/z, C88H112FN17O24)=906.2 [M/2+H]⁺.

### Intermediate L8rOPr1-d: tert-butyl (11R,14R,E)-11-(3-(5-((S)-2-amino-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

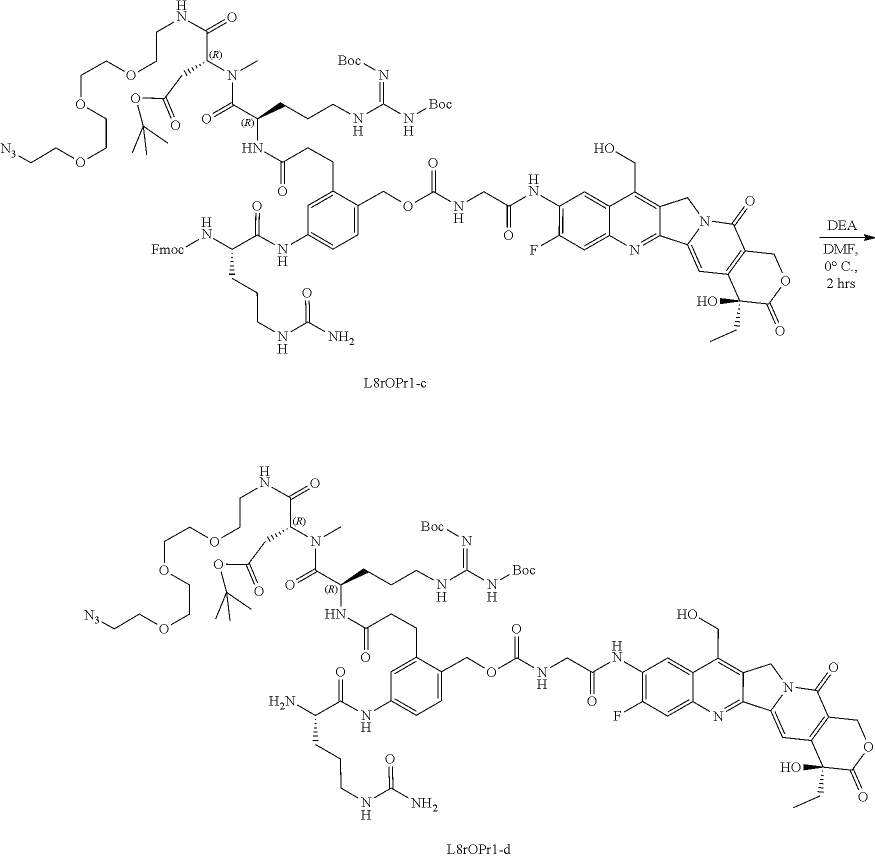

The synthesis procedures of intermediate L8rOPr1-d referred to the synthesis procedures of intermediate L8rSP2-g. Using intermediate L8rOPr1-c (tert-butyl (11R,14R,E)-11-(3-(5-((S)-2-((((9H-fluoren-9-yl)methoxy)carbonyl)amino)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 750 mg, 0.41 mmol) as the starting material, the desirable intermediate L8rOPr1-d (tert-butyl (11R,14R,E)-11-(3-(5-((S)-2-amino-5-ureidopentanamido)-2-((((2-(((S′)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 400 mg, 0.252 mmol, 61.45% in yield) was obtained as a yellow solid.

LC-MS (m/z, C₇₃H₁₀₂FN₁₇O₂₂)=794.8 [M/2+H]⁺.

### Intermediate L8rOPr1-e: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

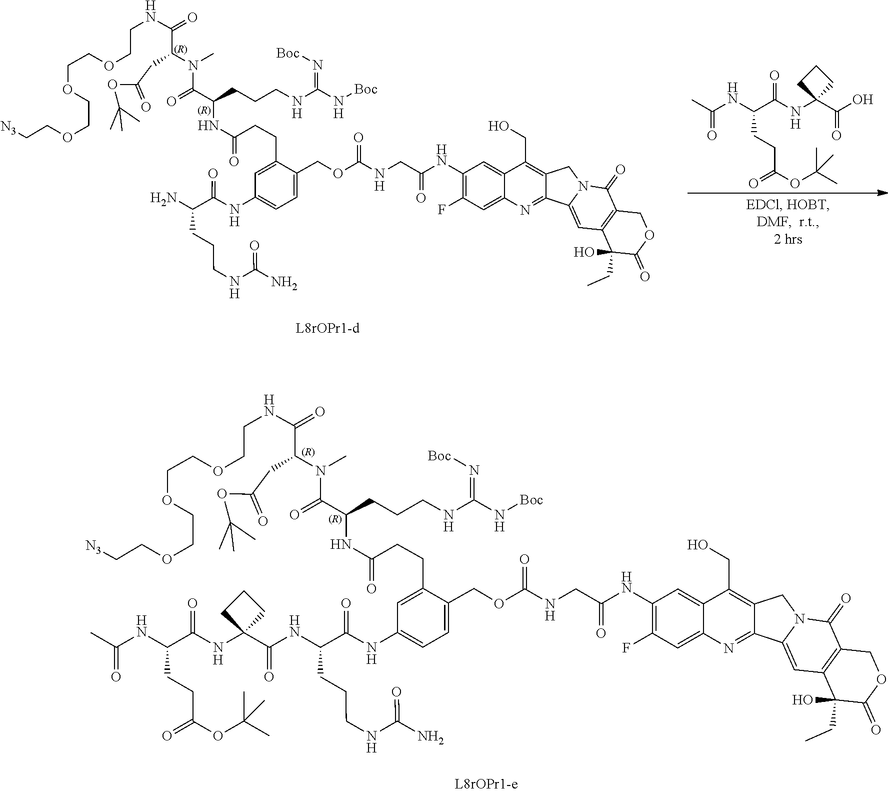

The synthesis procedures of intermediate L8rOPr1-e referred to the synthesis procedures of intermediate L8rSP2-h. Using intermediate L8rOPr1-d (tert-butyl (11R,14R,E)-11-(3-(5-((S)-2-amino-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 400 mg, 0.252 mmol) and intermediate HPH-L02 ((S)-1-(2-acetamido-5-(tert-butoxy)-5-oxopentanamido)cyclobutane-1-carboxylic acid; 103.92 mg, 0.304 mmol) as the starting material, the desirable intermediate L8rOPr1-e (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 350 mg, 0.183 mmol, 72.62% in yield) was obtained as a yellow solid.

LC-MS (m/z, C₈₉H₁₂₆FN₁₉O₂₇)=957.0 [M/2+H]⁺.

### Intermediate L8rOPr1-f: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

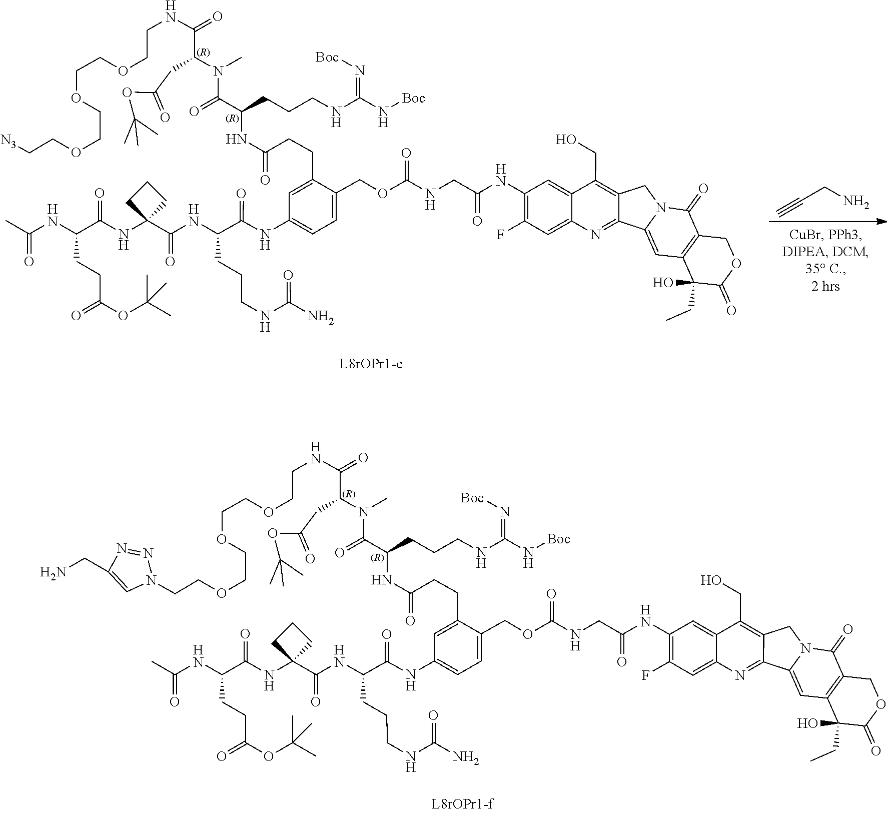

The synthesis procedures of intermediate L8rOPr1-f referred to the synthesis procedures of intermediate L8rSP2-i. Using intermediate L8rOPr1-e (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-azidoethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 170 mg, 0.0889 mmol) as the starting material, the desirable intermediate L8rOPr1-f (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 130 mg, 0.0661 mmol, 74.35% in yield) was obtained as a yellow solid.

LC-MS (m/z, C₉₂H₁₃₁FN₂₀O₂₇)=984.8 [M/2+H]⁺.

### Intermediate L8rOPr1-g: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate

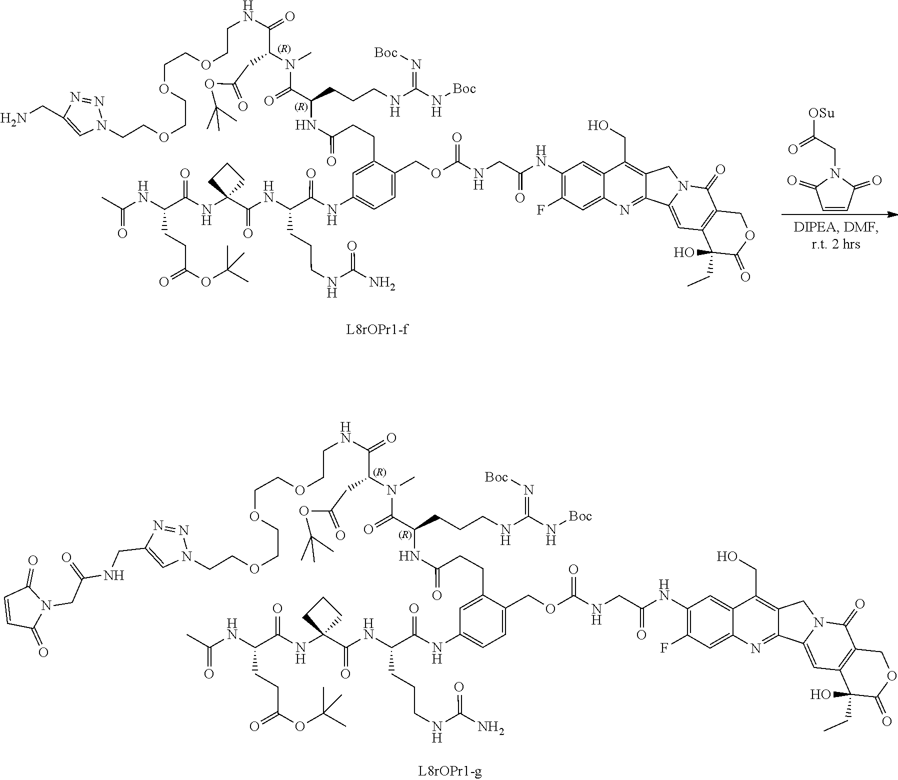

The synthesis procedures of intermediate L8rOPr1-g referred to the synthesis procedures of intermediate L8rSP2-j. Using intermediate L8rOPr1-f (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 45 mg, 0.0229 mmol) as the starting material, the desirable intermediate L8rOPr1-g (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 30 mg, 0.0143 mmol, 62.44% in yield) was obtained as a yellow solid.

LC-MS (m/z, C₉₈H₁₃₄FN₂₁O₃₀)=1053.0 [M/2+H]⁺.

### Linker-payload L8rOPr1: (R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

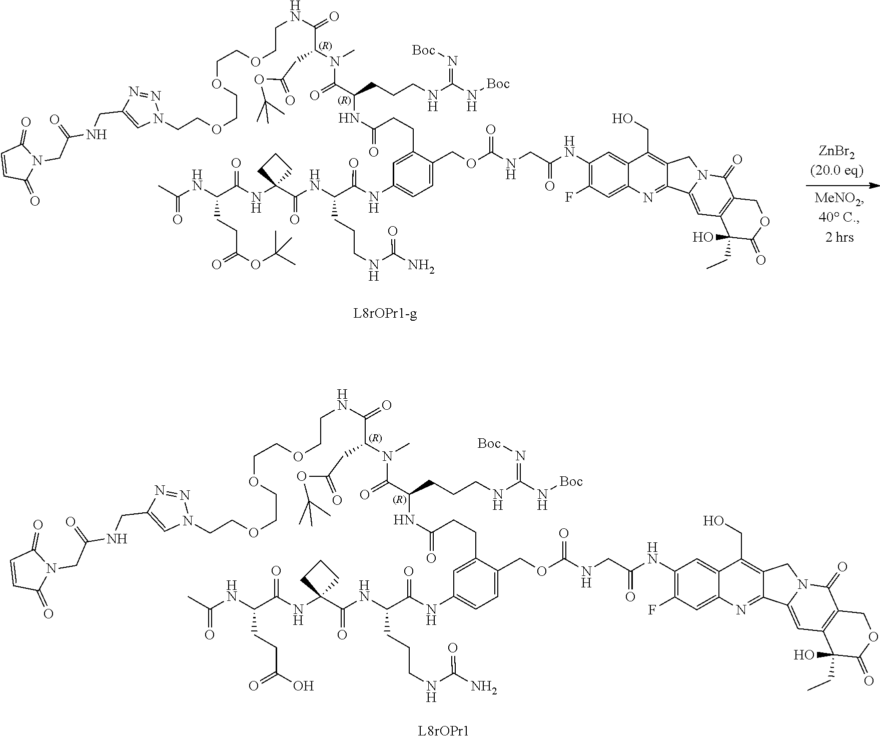

The synthesis procedures of linker-payload L8rOPr1 referred to the synthesis procedures of linker-payload L8rSP2. Using intermediate L8rOPr1-g (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-6-((tert-butoxycarbonyl)amino)-14-((2-(2-(2-(2-(4-((2-(2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-2,2,13-trimethyl-4,12-dioxo-3-oxa-5,7,13-triazahexadec-5-en-16-oate; 35 mg, 0.0166 mmol) as the starting material, the desirable linker-payload L8rOPr1 ((R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′: 6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 6 mg, 0.00335 mmol, 20.18% in yield) was obtained as a yellow solid.

LC-MS (m/z, C₈₀H₁₀₂FN₂₁O₂₆)=1791.7 [M+H]⁺.

### Linker-Payload L8rPPr1

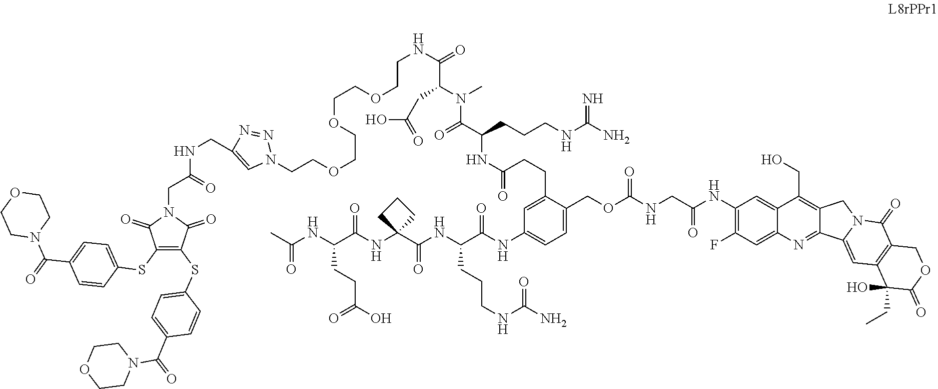

### Intermediate L8rPPr1-a: tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate

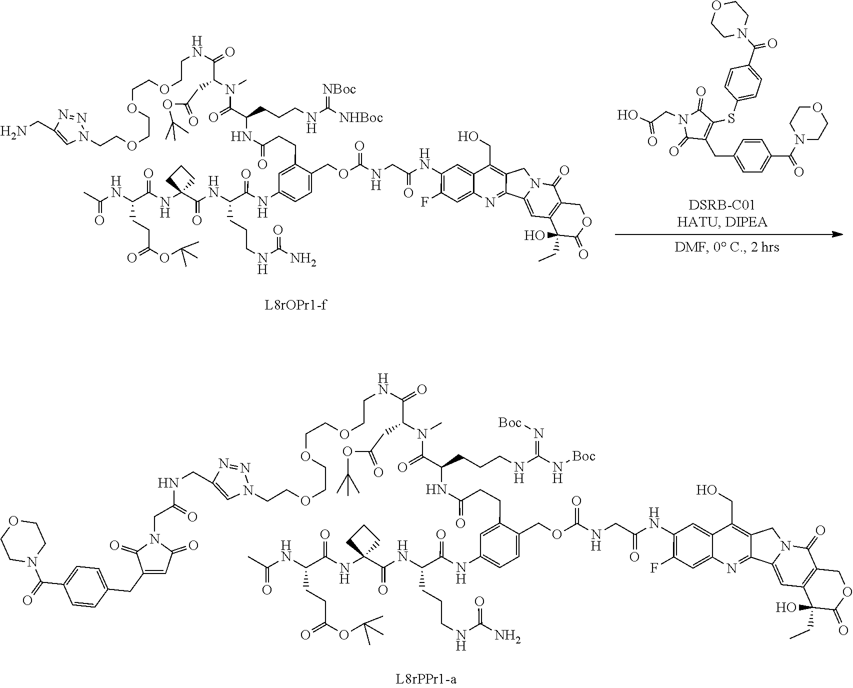

The synthesis procedures of intermediate L8rPPr1-a referred to the synthesis procedures of intermediate L8rTP2-a. Using intermediate L8rOPr1-f (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-(aminomethyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 60 mg, 0.0305 mmol) and intermediate DSRB-C01 (2-(3,4-bis((4-(morpholine-4-carbonyl)phenyl)thio)-2,5-dioxo-2,5-dihydro-1H-pyrrol-1-yl)acetic acid; 36 mg, 0.06 mmol) as the starting material, the desirable intermediate L8rPPr1-a (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 30 mg, 0.0118 mmol, 38.69% in yield) was obtained as a yellow solid.

LC-MS (m/z, C₉₈H₁₃₄FN₂₁O₃₀)=1274.1 [M/2+H]⁺.

### Linker-payload L8rPPr1: (R)-14-((R)-2-(3-(5-((S)-2-(1-((S)-2-acetamido-4-carboxybutanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid

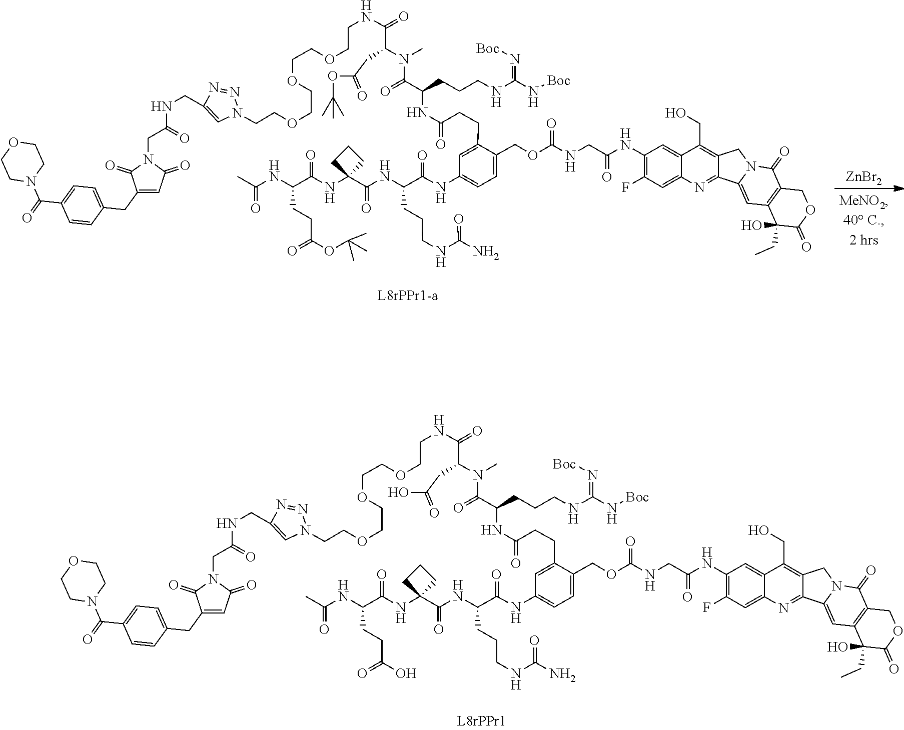

The synthesis procedures of linker-payload L8rPPr1 referred to the synthesis procedures of linker-payload L8rTP2. Using intermediate L8rPPr1-a (tert-butyl (11R,14R,E)-11-(3-(5-((S)-2-(1-((S)-2-acetamido-5-(tert-butoxy)-5-oxopentanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-14-((2-(2-(2-(2-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)ethoxy)ethoxy)ethoxy)ethyl)carbamoyl)-6-((tert-butoxycarbonyl)amino)-2,2,13-trimethyl-4,12-dioxo-3-oxa-5,7,13-triazahexadec-5-en-16-oate; 30 mg, 0.0118 mmol) as the starting material, the desirable linker-payload L8rPPr1 ((R)-14-((R)-2-(3-(5-((S′)-2-(1-((S′)-2-acetamido-4-carboxybutanamido)cyclobutane-1-carboxamido)-5-ureidopentanamido)-2-((((2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)carbamoyl)oxy)methyl)phenyl)propanamido)-5-guanidino-N-methylpentanamido)-1-(4-((2-(3,4-bis((4-(morpholine-4-carbonyl)phenyl)thio)-2,5-dioxo-2,5-dihydro-1H-pyrrol-1-yl)acetamido)methyl)-1H-1,2,3-triazol-1-yl)-13-oxo-3,6,9-trioxa-12-azahexadecan-16-oic acid; 8 mg, 0.00358 mmol, 30.34% in yield after purification) was obtained as a yellow solid.

LC-MS (m/z, C₁₀₂H₁₂₄FN₂₃O₃₀S₂)=1118.2 [M/2+H]⁺.

### Example 15. Preparation of Antibody for Conjugation

Antibodies with or without site-specific modification (thiomab, described in WO200603448 and DOI: 10.1021/bc500276m) were prepared with the following amino acid sequence below.

TABLE A
humanized antibody and bispecific antibody for conjugation

| Antibody | | |
|---|---|---|
| Name | Target | Amino acid sequence |
| Tsc | Her2 | SEQ ID No. 1 and SEQ ID No. 2 |
| v30384 | Folic acid | SEQ ID No. 3 and SEQ ID |
| | receptor alpha | No. 4 and SEQ ID No. 5 |
| FG1 | MUC1 | SEQ ID No. 6 and SEQ ID No. 7 |
| Gati | MUC1 | SEQ ID No. 8 and SEQ ID No. 9 |
| 186D | MUC1 | SEQ ID No. 10 and SEQ ID No. 11 |
| FG1-Tsc | Her2 & MUC1 | SEQ ID No. 12 and SEQ ID No. 13 |
| FG1-Tsc | Her2 & MUC1 | SEQ ID No. 20 and SEQ ID No. 21 |
| thiomab | | |
| Gati-Tsc | Her2 & MUC1 | SEQ ID No. 14 and SEQ ID No. 15 |
| 186D-Tsc | Her2 & MUC1 | SEQ ID No. 16 and SEQ ID No. 17 |
| DNP | Humanized IgG1 | SEQ ID No. 18 and SEQ ID No. 19 |
| | antibody as | |
| | control | |

cDNA was synthesized according to the amino acid sequences of the light chain and the heavy chain of each humanized antibody (i.e., the sequences as shown in SEQ ID NOs: 1, 2, 3, 4, 5, 6, 7, 8, 9, 10, 11, 12, 13, 14, 15, 16, 17, 20 and 21 in the sequence listing, respectively), and the cDNA fragment was inserted into the expression vector of the light chain or the heavy chain, respectively; cDNA was synthesized according to the amino acid sequences of the light chain variable region and the heavy chain variable region of the DNP antibody (i.e., the sequences as shown in SEQ ID NOs: 18 and 19 in the sequence listing, respectively), the heavy chain variable region cDNA was digested with FspAI and AfeI, and the light chain variable region cDNA was digested with FspAI and BsiwI, and then the cDNA fragment was inserted into the expression vector containing the signal peptide and the humanized heavy chain antibody IgG1 constant region and the expression vector containing the signal peptide and the humanized light chain antibody kappa constant region through FspAI/Afel or FspAI/BsiwI enzyme cutting sites, respectively.

The above recombinant plasmids were verified by sequencing, and the recombinant plasmids with improved purity were quantitatively extracted using an alkaline lysis kit (purchased from MACHEREY-NAGEL), with a mass of 500 μg or more, and filtered through a 0.22 μm filter membrane (purchased from Millopore) for transfection.

Before transfection, 293E cells (purchased from Invitrogen) were cultured in Freestyle 293 expression medium (purchased from Invitrogen). During transfection, 10% (v/v) F68 (purchased from Invitrogen) was added into Freestyle 293 expression medium until the final concentration of F68 was 0.1% (v/v), obtaining Freestyle 293 expression medium containing 0.1% (v/v) F68, namely medium A. 5 mL of medium A and 200 μg/mL PEI (purchased from Sigma) were mixed evenly to obtain medium B. 5 mL of medium A and 100 μg of heavy-chain and light-chain recombinant plasmids (the mass ratio of the heavy-chain recombinant plasmid to the light-chain recombinant plasmid was in the range of 1:1 to 1:3) were mixed to obtain medium C. After 5 minutes, medium B and medium C were combined and mixed evenly, then left to stand for 15 minutes to obtain mixture D. 10 mL of mixture D was slowly added to 100 mL of Freestyle 293 expression medium containing 293E cells until the cell density of 293E reached 1.5×10⁶ cells/mL, oscillating while adding to avoid excessive concentration of PEI, and placed in a shaker for cultivation, with the shaker set at 37° C., 130 RPM, and 8% CO₂ (v/v). On the second day, peptone was added to a final concentration of 0.5% (w/v). On days 5 to 7, the antibody titer of the culture supernatant was measured. On days 6 to 7, the supernatant was collected by centrifugation (3500 RPM, not less than 30 minutes), filtered through a 0.22 μm membrane, and the filtered cell supernatant was obtained for purification.

During antibody purification, for the continuously produced endotoxin-free chromatography column and Protein A resin (purchased from GE), 5 column volumes of 0.5 M NaOH solution were used for washing. Then, after being equilibrated to neutrality with 5 column volumes of PBS (PBS buffer, pH 7.4), the filtered cell supernatant was loaded onto the column, and the flow-through was collected when necessary. After the sample loading onto the column was completed, it was washed with PBS of 5 times the column volume. Elution was performed with 5 column volumes of 0.1 M Glycine-HCl at pH 3.0, the eluate was collected, and the antibody was immediately neutralized in the eluate by adding 0.1 volume of 1 M Tris-HCl buffer at pH 8.5. After monoclonal antibodies and bispecific antibodies were harvested, dialysis was carried out in 1×PBS buffer for 4 hours to avoid endotoxin contamination. After the dialysis was completed, the concentration was determined using spectrophotometry or the BCA kit, the antibody purity was measured using HPLC-SEC, and the antibody endotoxin content was detected using the endotoxin assay kit (purchased from Lonza). The obtained monoclonal antibodies and bispecific antibodies were characterized, and the quality inspection and identification results are shown in Table B below.

TABLE B
QC results for humanized monoclonal antibody or
bispecific antibody expression

| | Monomer purity by SEC- |
|---|---|
| Antibody Name | HPLC |
| Tsc (Trastuzumab) | 99.8% |
| v30384 | 96.5% |
| FG1 | 98.2% |
| Gati (Gatipotuzumab) | 99.5% |
| 186D | 96.4% |
| FG1-Tsc | 96.7% |
| FG1-Tsc thiomab | 97.2% |
| Gati-Tsc | 96.0% |
| 186D-Tsc | 97.5% |
| DNP | 98.7% |

### Example 16. Preparation of Antibody-Drug Conjugates

### Conjugation Steps of Monoclonal Antibody-Drug Conjugates and Bispecific Antibody-Drug Conjugates

The monoclonal antibody and bispecific antibody prepared in Example 15 were dialyzed into a PBS solution of 20 to 50 mM (pH value of 6.5 to 8.0), and the antibody concentration was diluted and adjusted to 5.0 to 10 mg/mL. A reducing reagent containing 6- to 12-fold molar ratio of tris(2-carboxyethyl)phosphine (TCEP hydrochloride; CAS RN: 51805-45-9) was added to the antibody to reduce the inter-chain disulfide bonds of the antibody. The antibody was incubated at 37° C. for 2 to 4 hours, then a stock solution in an organic solvent (such as DMSO, DMF, DMAc, or acetonitrile) containing 10- to 16-fold molar ratio of linker-payloads (for example, L7P1, L2P2, L3P2, L7P2, L8rP2, L8rQP2, L8rRP2, L8rSP2, and L8rTP2, previously described linker-payloads) was added to the reduced antibody and mixed evenly, and incubation was continued at 25° C. for another 2 to 4 hours. Excess tris(2-carboxyethyl)phosphine and linker-payload were removed through a desalting column or by dialysis. The obtained conjugates were subsequently filtered through a 0.22 μm membrane for use in in vivo and in vitro experiments. The DAR value of the conjugate was 4 (disulfide-bridged conjugation) or 8 (full interchain disulfide conjugation). In addition, linker-payloads such as GGFG-Dxd (CAS RN: 1599440-13-7), which could be purchased, were obtained from MedChemExpress (Shanghai, China). The conjugation method of GGFG-Dxd conjugate was the same as the above conjugation method.

### Conjugation Steps of Site-Specific Mutated (Cysteine) Antibodies

In Example 15, the antibody that had been modified by cysteine site-specific modification was dialyzed into a 20 to 50 mM PBS solution (pH value of 6.5 to 8.0), and the antibody concentration was diluted and adjusted to 5.0 to 10 mg/mL. A reducing reagent containing 15- to 30-fold molar ratio of tris(2-carboxyethyl)phosphine (TCEP hydrochloride; CAS RN: 51805-45-9) was added to the antibody to reduce the inter-chain disulfide bonds and the disulfide bonds at the sites modified by cysteine of the antibody. After incubation at 37° C. for 2 hours, the reduced antibody was re-dialyzed into a fresh 20 to 50 mM PBS solution (pH value of 6.5 to 8.0), and dehydroascorbic acid (CAS RN: 490-83-5) was added. The antibody was incubated at 37° C. for two hours. After the reoxidized antibody was dialyzed into a fresh 20 to 50 mM PBS solution (pH value of 6.5 to 8.0), a stock solution in an organic solvent (such as DMSO, DMF, DMAc, or acetonitrile) containing linker-payloads (for example, L7P1, L2P2, L3P2, L7P2, L8rP2, L8rQP2, L8rRP2, L8rSP2, and L8rTP2, previously described linker-payloads) at a 6- to 8-fold molar ratio was added to the antibody and mixed evenly. The antibody was incubated at 25° C. for 2 to 4 hours. The excess linker-payload in the solution was removed by a desalting column or dialysis. The obtained conjugates were subsequently filtered through a 0.22 μm membrane for use in in vivo and in vitro experiments. In addition, linker-payloads such as GGFG-Dxd (CAS RN: 1599440-13-7), which could be purchased, were obtained from MedChemExpress (Shanghai, China). The conjugation method of GGFG-Dxd conjugate was the same as the above conjugation method.

The antibody-drug conjugates prepared are as described in Table C

TABLE C
ADC planned to be generated

| | | | Linker- | Aimed |
|---|---|---|---|---|
| | | | payload | DAR |
| ADC | Antibody | Antibody | used for | value for |
| name | name | sequence | conjugation | conjugation |
| ADC-1a | TSc | SEQ ID No. 1 & | L7P1 | 8 |
| ADC-1b | (Trastuzumab) | SEQ ID No. 2 | L2P2 | 8 |
| ADC-1c | | | L3P2 | 8 |
| ADC-1d | | | L7P2 | 8 |
| ADC-1r1 | | | MC-GGFG- | 8 |
| | | | Dxd | |
| ADC-2a | FG1 | SEQ ID No. 6 & | L7P1 | 8 |
| ADC-2b | | SEQ ID No. 7 | L2P2 | 8 |
| ADC-2c | | | L3P2 | 8 |
| ADC-2d | | | L7P2 | 8 |
| ADC-3a | FG1-Tsc | SEQ ID No. 20 | L7P1 | 8 |
| ADC-3b | Thiomab | & SEQ ID | L2P2 | 8 |
| ADC-3c | | No. 21 | L3P2 | 8 |
| ADC-3d | | | L7P2 | 8 |
| ADC-4a | DNP | SEQ ID No. 18 | L7P1 | 8 |
| ADC-4b | | & SEQ ID | L2P2 | 8 |
| ADC-4c | | No. 19 | L3P2 | 8 |
| ADC-4d | | | L7P2 | 8 |
| ADC-4r1 | | | MC-GGFG- | 8 |
| | | | Dxd | |
| ADC-1e | TSc | SEQ ID No. 1 & | L3aP1 | 8 |
| ADC-1f | (Trastuzumab) | SEQ ID No. 2 | L3aP2 | 8 |
| ADC-1g | | | L8rP2 | 8 |
| ADC-1h | | | L18aP2 | 8 |
| ADC-4e | DNP | SEQ ID No. 18 | L3aP1 | 8 |
| ADC-4f | | & SEQ ID | L3aP2 | 8 |
| ADC-4g | | No. 19 | L8rP2 | 8 |
| ADC-4h | | | L18aP2 | 8 |
| ADC-1i | TSc | SEQ ID No. 1 & | L8rQP2 | 8 |
| ADC-1j | (Trastuzumab) | SEQ ID No. 2 | L8rRP2 | 8 |
| ADC-1k | | | L8rSP2 | 8 |
| ADC-1l | | | L8rTP2 | 8 |
| ADC-1IR | | | L8rMPr1 | 8 |
| ADC-1JR | | | L8rNPr1 | 8 |
| ADC-1KR | | | L8rOPr1 | 8 |
| ADC-1LR | | | L8rPPr1 | 8 |
| ADC-2i | FG1 | SEQ ID No. 6 & | L8rQP2 | 8 |
| ADC-2j | | SEQ ID No. 7 | L8rRP2 | 8 |
| ADC-2k | | | L8rSP2 | 8 |
| ADC-2l | | | L8rTP2 | 8 |
| ADC-2IR | | | L8rMPr1 | 8 |
| ADC-2JR | | | L8rNPr1 | 8 |
| ADC-2KR | | | L8rOPr1 | 8 |
| ADC-2LR | | | L8rPPr1 | 8 |
| ADC-4i | DNP | SEQ ID No. 18 | L8rQP2 | 8 |
| ADC-4j | | & SEQ ID | L8rRP2 | 8 |
| ADC-4k | | No. 19 | L8rSP2 | 8 |
| ADC-4l | | | L8rTP2 | 8 |
| ADC-4IR | | | L8rMPr1 | 8 |
| ADC-4JR | | | L8rNPr1 | 8 |
| ADC-4KR | | | L8rOPr1 | 8 |
| ADC-4LR | | | L8rPPr1 | 8 |
| ADC-5f | Gati | SEQ ID No. 8 & | L3aP2 | 8 |
| ADC-5g | | SEQ ID No. 9 | L8rP2 | 8 |
| ADC-5r1 | | | MC-GGFG- | 8 |
| | | | Dxd | |

### LCMS Method for Drug to Antibody Ratio (DAR) of ADC Determination

LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 A, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min) at a flow rate of 0.5 mL/min
Injected sample amount: 10 μg
MS analysis conditions: Intact and denaturing MS data were acquired in HMR mode at a setting of R=15k and deconvolved using the ReSpect™ algorithm and Sliding Window integration in Thermo Scientific™ BioPharma Finder™ 4.0 software.

### SEC Method for ADC Purity

The analytical parameters for SEC are shown below:
HPLC system: Waters H-Class UPLC System; Detector: measurement wavelength: 280 nm
Column: ACQUITY UPLC BEH200 SEC 1.7 μm 4.6×150 mm, Waters
Column temperature: room temperature; mobile phase A: 50 mM Phosphate buffer, 15% isopropyl alcohol, pH 7.0
Gradient program: under 10 min isocratic elutions with the flow rate of 0.3 mL/min

TABLE D
QC results for ADC generated

| | | Linker-payload | DAR | Monomer |
|---|---|---|---|---|
| ADC | Antibody | used for | value by | purity by |
| name | name | conjugation | LC-MS | SEC-HPLC |
| ADC-1a | TSc | L7P1 | 7.2 | 96.7% |
| ADC-1b | (trastuzumab) | L2P2 | 7.4 | 97.0% |
| ADC-1c | | L3P2 | 6.9 | 97.2% |
| ADC-1d | | L7P2 | 7.4 | 96.3% |
| ADC-1r1 | | MC-GGFG-Dxd | 8.0 | 98.8% |
| ADC-2a | FG1 | L7P1 | 7.4 | 96.4% |
| ADC-2b | | L2P2 | 7.3 | 97.2% |
| ADC-2c | | L3P2 | 6.8 | 97.3% |
| ADC-2d | | L7P2 | 7.3 | 96.5% |
| ADC-2r1 | | MC-GGFG-Dxd | 7.9 | 98.7% |
| ADC-3a | | L7P1 | 3.4 | 96.2% |
| ADC-3b | FG1-Tsc | L2P2 | 3.3 | 97.2% |
| ADC-3c | Thiomab | L3P2 | 3.5 | 98.0% |
| ADC-3d | | L7P2 | 3.2 | 96.1% |
| ADC-4a | DNP | L7P1 | 7.6 | 96.4% |
| ADC-4b | | L2P2 | 7.4 | 96.8% |
| ADC-4c | | L3P2 | 7.3 | 97.1% |
| ADC-4d | | L7P2 | 7.6 | 96.5% |
| ADC-4r1 | | GGFG-Dxd | 7.9 | 98.9% |
| ADC-1e | TSc | L3aP1 | 7.5 | 97.6% |
| ADC-1f | | L3aP2 | 7.6 | 97.9% |
| ADC-1g | | L8rP2 | 7.8 | 98.2% |
| ADC-1h | | L18aP2 | 7.9 | 97.4% |
| ADC-4e | DNP | L3aP1 | 7.4 | 98.1% |
| ADC-4f | | L3aP2 | 7.6 | 97.8% |
| ADC-4g | | L8rP2 | 7.2 | 97.6% |
| ADC-4h | | L18aP2 | 7.5 | 98.3% |
| ADC-1i | TSc | L8rQP2 | 7.4 | 98.9% |
| ADC-1j | | L8rRP2 | 3.8 | 99.1% |
| ADC-1k | | L8rSP2 | 7.1 | 98.3% |
| ADC-1l | | L8rTP2 | 3.9 | 98.6% |
| ADC-1IR | | L8rMPr1 | 7.4 | 97.9% |
| ADC-1JR | | L8rNPr1 | 3.7 | 98.2% |
| ADC-1KR | | L8rOPr1 | 7.6 | 98.5% |
| ADC-1LR | | L8rPPr1 | 4.1 | 98.7% |
| ADC-2i | FG1 | L8rQP2 | 7.9 | 98.4% |
| ADC-2j | | L8rRP2 | 3.6 | 98.1% |
| ADC-2k | | L8rSP2 | 7.8 | 97.6% |
| ADC-2l | | L8rTP2 | 3.8 | 98.3% |
| ADC-2IR | | L8rMPr1 | 7.0 | 99.1% |
| ADC-2JR | | L8rNPr1 | 3.9 | 99.2% |
| ADC-2KR | | L8rOPr1 | 7.6 | 98.7% |
| ADC-2LR | | L8rPPr1 | 4.0 | 98.5% |
| ADC-4i | DNP | L8rQP2 | 7.3 | 98.1% |
| ADC-4j | | L8rRP2 | 4.1 | 98.2% |
| ADC-4k | | L8rSP2 | 7.6 | 97.9% |
| ADC-4l | | L8rTP2 | 3.9 | 98.6% |
| ADC-4IR | | L8rMPr1 | 7.6 | 98.5% |
| ADC-4JR | | L8rNPr1 | 3.8 | 98.6% |
| ADC-4KR | | L8rOPr1 | 7.1 | 98.7% |
| ADC-4LR | | L8rPPr1 | 3.9 | 98.9% |
| ADC-5f | Gati | L3aP2 | 7.5 | 98.6% |
| ADC-5g | | L8rP2 | 7.9 | 98.3% |
| ADC-5r1 | | MC-GGFG-Dxd | 7.6 | 99.0% |

### Effect Test:

### Example 17. Cell Viability Assays for Camptothecin Analogues and ADCs

Dxd (CAS RN: 1599440-33-1) as a camptothecin payload reference was purchased from MedChemExpress (MedChemExpress, HY-13631D). The in vitro efficacy of the camptothecin analogues and ADCs was measured by evaluating their cytotoxic activity on various cell lines. This assay was conducted in clear 96-well cell culture plates (Greiner: 655090). The cell lines used were SK-OV-3 (human ovarian serous cystadenocarcinoma), BT-474 (human mammary ductal carcinoma), AsPC-1 (human pancreatic ductal adenocarcinoma, ABCG2-expressing), JIMT-1 (human breast carcinoma), HEK-293 cell line, NCI-N87 (human gastric carcinoma), SK-BR-3 (human breast adenocarcinoma) and CHO-K1 (Chinese hamster ovarian cell line).

Compound P1, compound P2, and the above-mentioned different antibody-drug conjugates were respectively subjected to gradient dilution with complete medium (5-fold gradient dilution), and 100 μL of cell line was added to a 96-well cell culture plate at a density of 1,000 to 3,000 cells per well. After culturing the cell suspension overnight in a 37° C. incubator (with 5% CO₂), 10 μL of different concentrations of purified antibody-drug conjugates, or dilutions of compound P1 or compound P2 were added to each well. The cells were then incubated at 37° C. (with 5% CO₂) for another 4 to 6 days. Cell viability was then assessed using the CellTiter-Glo kit (purchased from Promega; refer to the product instructions for usage). The data of cell viability detection were presented as viability percentages based on blank control wells, as described in Table E.1, Table F.1, Table F.2, and Table F.3.

TABLE E.1
cytotoxicity of camptothecin analogues

| | IC₅₀ (nM) | | |
|---|---|---|---|
| Cell lines | P1 | P2 | Dxd |
| SK-OV-3 | 1.42 | 0.92 | 8.45 |
| (96 hours incubation) | | | |
| BT-474 | 40.2 | 38.7 | >250 |
| (96 hours incubation) | | | |
| AsPC-1 | 2.57 | 0.99 | 22.44 |
| (96 hours incubation) | | | |
| JIMT-1 | 28.22 | 5.98 | >250 |
| (96 hours incubation) | | | |

TABLE F.1
cytotoxicity of ADC samples

| | IC₅₀ (nM) | | |
|---|---|---|---|
| Cell lines | ADC-3c | ADC-1c | ADC-4C |
| SK-OV-3 | 0.77 | 2.83 | 155.60 |
| (120 hours incubation) | | | |
| HEK-293 (null) | 45.54 | 75.65 | 81.99 |
| (120 hours incubation) | | | |

TABLE F.2
cytotoxicity of ADC samples

| | IC₅₀ (nM) | | | | | | | |
|---|---|---|---|---|---|---|---|---|
| | ADC- | ADC- | ADC- | ADC- | ADC- | ADC- | ADC- | ADC- |
| Cell lines | 1r1 | 1f | 1g | 1h | 4r1 | 4f | 4g | 4h |
| NCI-N87 | 0.198 | 0.497 | 0.528 | N/A | >250 | >250 | >250 | N/A |
| (120 hours | | | | | | | | |
| incubation) | | | | | | | | |
| SK-BR-3 | 0.98 | 0.08 | 0.262 | 0.261 | >250 | >250 | >250 | >250 |
| (120 hours | | | | | | | | |
| incubation) | | | | | | | | |
| CHO-K1 (null) | >250 | >250 | >250 | >250 | >250 | >250 | >250 | >250 |
| (120 hours | | | | | | | | |
| incubation) | | | | | | | | |

TABLE F.3
cytotoxicity of ADC samples

| | IC₅₀ (nM) | | | |
|---|---|---|---|---|
| Cell lines | ADC-1k | ADC-11 | ADC-4k | ADC-41 |
| NCI-N87 | 0.246 | 0.186 | >250 | >250 |
| (120 hours | | | | |
| incubation) | | | | |
| CHO-K1 (null) (120 | >250 | >250 | >250 | >250 |
| hours incubation) | | | | |

### Example 18. In Vivo Efficacy Studies of Resulting ADCs in SK-OV-3 Cell-Derived Xenograft (CDX) Mouse Model

In SK-OV-3 CDX mouse model, the cells were suspended in the basal medium and mixed with 50% Matrigel® (8×10⁷) 100 μL, which were inoculated subcutaneously into the right flank of BALB/c mice. When the tumors grew to 160 mm³, the mice were randomly divided into seven groups according to the tumor volume, with five mice per group, as shown in Tables G.1, G.2, or G.3.

On the first day of the experiment, different doses of antibody-drug conjugates (such as ADC-4c, ADC-1r1, ADC1c, ADC2c, ADC-5f, ADC-5g, ADC-5r) and PBS vehicle control were injected into the tail vein. And the administration was carried out once on day 14, with a total of two administrations, as shown in Table G.1, Table G.2, and Table G.3. Tumor volume and mouse weight were measured twice a week to record data.

The formula for calculating tumor volume (TV) was as follows: TV=0.5×a×b₂, in which a (mm) was expressed as the length of the tumor measured with a vernier caliper, and b (mm) was expressed as the width of the tumor measured with a vernier caliper.

The formula for calculating tumor growth inhibition (TGI) was as follows:

$$TGI = \frac{1 - ( {TV}_{t} - T {V}_{initial} )}{{CTV}_{t} - {CTV}_{initial}} \times 100 \% ,$$

in which TVₜ was the average tumor volume of mice on the measurement day in the antibody-drug conjugate test group at a certain dose; TVᵢₙᵢₜᵢₐₗ was the average tumor volume of mice in the antibody-drug conjugate test group at that dose on day 0 at the start of the experiment; CTVₜ was the average tumor volume of mice on the measurement day in the PBS vehicle control group; CTVᵢₙᵢₜᵢₐₗ was the average tumor volume of mice in the PBS vehicle control group on day 0 at the start of the experiment.

TABLE G.1
in vivo activity of ADCs in the SK-OV-3 CDX mouse model

| | Linker- | | | | |
|---|---|---|---|---|---|
| | payloads in | | | | |
| | antibody- | Administration | Average tumor size on | Average tumor | |
| Experimental | drug | schedule and | Day 14 | size on Day 28 | TGI on |
| grouping | conjugates | dosage | (mm³) ± SEM | (mm³) ± SEM | Day 28 |
| Vehicle | N/A | i.v., | 522.52 ± 67.53 | 1398.67 ± 140.46 | N/A |
| (PBS | | Q2W × 2 | | | |
| control) | | | | | |
| ADC-4c | L3P2 | i.v., | 325.43 ± 69.66 | 842.44 ± 269.89 | 44.93% |
| | | 8 mg/kg, | | | |
| | | Q2W × 2 | | | |
| ADC-1r1 | MC- | 3 mg/kg, | 231.60 ± 52.07 | 347.89 ± 83.45 | 85.09% |
| | GGFG- | Q2W × 2 | | | |
| | Dxd | | | | |
| ADC-1c | L3P2 | i.v., | 141.46 ± 17.63 | 102.57 ± 9.58 | 105.15% |
| | | 3 mg/kg, | | | |
| | | Q2W × 2 | | | |
| ADC-1c | L3P2 | 8 mg/kg, | 104.34 ± 11.22 | 65.89 ± 3.73 | 107.98% |
| | | Q2W × 2 | | | |
| ADC-2c | L3P2 | i.v., | 171.33 ± 23.71 | 177.55 ± 25.87 | 78.30% |
| | | 3 mg/kg, | | | |
| | | Q2W × 2 | | | |
| ADC-2c | L3P2 | i.v., | 288.27 ± 31.02 | 499.99 ± 92 | 98.97% |
| | | 8 mg/kg, | | | |
| | | Q2W × 2 | | | |

As shown in Table G.1, all antibody-drug conjugates tested in the SK-OV-3 CDX mouse model were observed to have achieved good tumor growth inhibition effects on day 28 after the start of the experiment.

TABLE G.2
in vivo activity of antibody-drug conjugates in the
SK-OV-3 CDX mouse model

| | Linker- | | Average tumor | |
|---|---|---|---|---|
| Experi- | payloads in | Administration | size on Day 31 | |
| mental | antibody-drug | schedule | (mm³) ± | TGI on |
| grouping | conjugates | and dosage | SEM | Day 31 |
| Vehicle | N/A | i.v., Q2W × 2 | 1388.22 ± 207.09 | N/A |
| (PBS control) | | | | |
| ADC-5r1 | MC-GGFG- | i.v., 8 mg/kg, | 646.15 ± 41.14 | 59.08% |
| | Dxd | Q2W × 2 | | |
| ADC-5g | L8rP2 | i.v., 8 mg/kg, | 292.12 ± 75.08 | 87.39% |
| | | Q2W × 2 | | |

As shown in Table G.2, the antibody-drug conjugate ADC-5g tested in the SK-OV-3 CDX mouse model was observed to have achieved a better in vivo tumor growth inhibition effect than ADC-5r1 on day 31 after the start of the experiment.

TABLE G.3
in vivo activity of antibody-drug conjugates in the
SK-OV-3 CDX mouse model

| | Linker- | | Average | |
|---|---|---|---|---|
| | payloads | | tumor size | |
| | in antibody- | Administration | on Day 28 | |
| Experimental | drug | schedule | (mm³) ± | TGI on |
| grouping | conjugates | and dosage | SEM | Day 28 |
| Vehicle | N/A | i.v., Q2W × 2 | 942.53 ± 70.77 | N/A |
| (PBS | | | | |
| control) | | | | |
| ADC-2k | L8rSP2 | i.v., 3 mg/kg, | 136.41 ± 21.55 | 97.49% |
| | | Q2W × 2 | | |
| ADC-4k | L8rSP2 | i.v., 3 mg/kg, | 630.39 ± 149.69 | 37.83% |
| | | Q2W × 2 | | |
| ADC-4l | L8rTP2 | i.v., 3 mg/kg, | 892.91 ± 112.76 | 6.10% |
| | | Q2W × 2 | | |
| ADC-2i | L8rQP2 | i.v., 3 mg/kg, | 335.77 ± 59.55 | 73.36% |
| | | Q2W × 2 | | |
| ADC-2j | L8rRP2 | i.v., 3 mg/kg, | 393.55 ± 102.54 | 66.29% |
| | | Q2W × 2 | | |
| ADC-4i | L8rQP2 | i.v., 3 mg/kg, | 719.90 ± 39.55 | 26.91% |
| | | Q2W × 2 | | |
| ADC-4j | L8rRP2 | i.v., 3 mg/kg, | 757.74 ± 43.33 | 22.28% |
| | | Q2W × 2 | | |
| ADC-2IR | L8rMPr1 | i.v., 3 mg/kg, | 598.69 ± 71.17 | 41.59% |
| | | Q2W × 2 | | |
| ADC-2KR | L8rOPr1 | i.v., 3 mg/kg, | 503.31 ± 103.77 | 53.11% |
| | | Q2W × 2 | | |
| ADC-4IR | L8rMPr1 | i.v., 3 mg/kg, | 746.62 ± 131.03 | 23.77% |
| | | Q2W × 2 | | |
| ADC-4KR | L8rOPr1 | i.v., 3 mg/kg, | 646.45 ± 142.30 | 35.78% |
| | | Q2W × 2 | | |
| ADC-2JR | L8rNPr1 | i.v., 3 mg/kg, | 636.29 ± 46.03 | 36.97% |
| | | Q2W × 2 | | |
| ADC-4JR | L8rNPr1 | i.v., 3 mg/kg, | 720.31 ± 95.62 | 26.85% |
| | | Q2W × 2 | | |
| ADC-4LR | L8rPPr1 | i.v., 3 mg/kg, | 811.27 ± 113.66 | 15.88% |
| | | Q2W × 2 | | |
| ADC-2r1 | MC-GGFG- | i.v., 3 mg/kg, | 590.18 ± 51.15 | 42.71% |
| | Dxd | Q2W × 2 | | |
| ADC-4r1 | MC-GGFG- | i.v., 3 mg/kg, | 675.28 ± 141.61 | 32.42% |
| | Dxd | Q2W × 2 | | |

As shown in Table G.3, all antibody-drug conjugates ADC-2k, ADC-2i, and ADC-2j tested in the SK-OV-3 CDX mouse model were observed to have achieved better in vivo tumor growth inhibition effects than ADC-2MR, ADC-2KR, ADC-2JR, and ADC-2r1 on day 24 after the start of the experiment.

### Example 19: In Vivo Efficacy of Antibody-Drug Conjugates in the JIMT-1 (Her2-Positive Human Breast Cancer) CDX Mouse Model

In JIMT-1 CDX mouse model, the cells were suspended in the basal medium and mixed with 5000 Matrigel® (1×10⁷) 100 μL, which were inoculated subcutaneously into the right flank of female BALB/c mice. When the tumors grew to 180 mm³, the mice were randomly divided into seven groups according to the tumor volume, with five mice per group, as shown in Table G.4 or G.5.

On day 1 (Table G.4 and Table G.5) and day 14 (Table G.5) of the experiment, different doses of antibody-drug conjugates (such as ADC-1r1, ADC-1c, ADC-4c) and PBS vehicle control were injected into the tail vein, administered 1 or 2 times in total as shown in Table G.4 and G.5. Tumor volume and mouse weight were measured twice a week to record data. The formula for calculating tumor volume (TV) was as follows:

$$TV = 0.5 \times a \times {b}^{2}$$

in which a (mm) was expressed as the length of the tumor measured with a vernier caliper, and b (mm) was expressed as the width of the tumor measured with a vernier caliper.

The formula for calculating tumor growth inhibition (TGI) was as follows:

$$TGI = \frac{1 - ( {TV}_{t} - T {V}_{initial} )}{{CTV}_{t} - {CTV}_{initial}} \times 100 \%$$

in which TVₜ was the average tumor volume of mice on the measurement day in the antibody-drug conjugate test group at a certain dose; TVᵢₙᵢₜᵢₐₗ was the average tumor volume of mice in the antibody-drug conjugate test group at that dose on day 0 at the start of the experiment; CTVₜ was the average tumor volume of mice on the measurement day in the PBS vehicle control group; CTVᵢₙᵢₜᵢₐₗ was the average tumor volume of mice in the PBS vehicle control group on day 0 at the start of the experiment.

TABLE G.4
in vivo activity of antibody-drug conjugates in the
JIMT-1 CDX mouse model

| | Linker- | | | |
|---|---|---|---|---|
| | payloads | | | |
| | in | | Average tumor | |
| Experi- | antibody- | Administration | size on Day 35 | TGI |
| mental | drug | schedule and | (mm³) ± | on Day |
| grouping | conjugates | dosage | SEM | 35 |
| Vehicle | N/A | i.v., | 1513.14 ± 259.85 | N/A |
| (PBS | | single dose | | |
| control) | | | | |
| ADC-4c | L3P2 | i.v., | 1175.61 ± 169.20 | 25.31% |
| | | 3 mg/kg, single | | |
| | | dose | | |
| ADC-1r1 | MC- | i.v., 3 mg/kg, | 810.08 ± 157.19 | 52.82% |
| | GGFG- | single dose | | |
| | Dxd | | | |
| ADC-1c | L3P2 | i.v., | 130.29 ± 49.38 | 103.82% |
| | | 3 mg/kg, single | | |
| | | dose | | |

As shown in Table G.4, all antibody-drug conjugates ADC-1c tested in the JIMT-1 (Her2-positive, drug-resistant breast cancer cell) CDX mouse model were observed to have achieved better tumor growth inhibition effects than other antibody-drug conjugates ADC-4c and ADC-1r1 on day 35 day after the start of the experiment.

TABLE G.5
in vivo activity of antibody-drug conjugates in the
JIMT-1 CDX mouse model

| | Linker- | | | |
|---|---|---|---|---|
| | payloads | | Average tumor | |
| Experi- | in antibody- | Administration | size on Day 37 | TGI on |
| mental | drug | schedule | (mm³) ± | Day |
| grouping | conjugates | and dosage | SEM | 37 |
| Vehicle | N/A | i.v., | 919.21 ± 122.43 | N/A |
| (PBS | | Q2W×2 | | |
| control) | | | | |
| ADC-1k | L8rSP2 | i.v., | 56.08 ± 15.68 | 116.28% |
| | | 3 mg/kg, | | |
| | | Q2W × 2 | | |
| ADC-11 | L8rTP2 | i.v., | 138.53 ± 43.14 | 105.47% |
| | | 3 mg/kg, | | |
| | | Q2W × 2 | | |
| ADC-4k | L8rSP2 | i.v., | 849.56 ± 95.37 | 9.41% |
| | | 3 mg/kg, | | |
| | | Q2W × 2 | | |
| ADC-4l | L8rTP2 | i.v., | 879.41 ± 128.53 | 5.18% |
| | | 3 mg/kg, | | |
| | | Q2W × 2 | | |
| ADC-1i | L8rQP2 | i.v., | 128.40 ± 44.09 | 106.35% |
| | | 3 mg/kg, | | |
| | | Q2W × 2 | | |
| ADC-1j | L8rRP2 | i.v., | 276.71 ± 20.61 | 86.35% |
| | | 3 mg/kg, | | |
| | | Q2W × 2 | | |
| ADC-4i | L8rQP2 | i.v., | 906.71 ± 62.20 | 1.45% |
| | | 3 mg/kg, | | |
| | | Q2W × 2 | | |
| ADC-4j | L8rRP2 | i.v., | 912.15 ± 116.61 | 0.89% |
| | | 3 mg/kg, | | |
| | | Q2W × 2 | | |
| ADC-1IR | L8rMPr1 | i.v., | 736.09 ± 66.43 | 24.85% |
| | | 3 mg/kg, | | |
| | | Q2W × 2 | | |
| ADC-1KR | L8rOPr1 | i.v., | 557.13 ± 63.07 | 48.59% |
| | | 3 mg/kg, | | |
| | | Q2W × 2 | | |
| ADC-4IR | L8rMPr1 | i.v., | 946.55 ± 101.29 | −3.51% |
| | | 3 mg/kg, | | |
| | | Q2W × 2 | | |
| ADC-4KR | L8rOPr1 | i.v., | 1127.89 ± 173.22 | −28.19% |
| | | 3 mg/kg, | | |
| | | Q2W × 2 | | |
| ADC-1JR | L8rNPr1 | i.v., | 942.05 ± 146.60 | −3.33% |
| | | 3 mg/kg, | | |
| | | Q2W × 2 | | |
| ADC-1LR | L8rPPr1 | i.v., | 546.97 ± 79.36 | 49.91% |
| | | 3 mg/kg, | | |
| | | Q2W × 2 | | |
| ADC-4JR | L8rNPr1 | i.v., | 1266.39 ± 345.39 | −46.98% |
| | | 3 mg/kg, | | |
| | | Q2W × 2 | | |
| ADC-4LR | L8rPPr1 | i.v., | 1302.13 ± 402.37 | −51.65% |
| | | 3 mg/kg, | | |
| | | Q2W × 2 | | |
| ADC-4r1 | MC-GGFG- | i.v., | 952.48 ± 144.55 | −4.76% |
| | Dxd | 3 mg/kg, | | |
| | | Q2W × 2 | | |

As shown in Table G.5, all antibody-drug conjugates ADC-1i, ADC-1j, ADC-1k, and ADC-1l tested in the JIMT-1 (Her2-positive human breast cancer) CDX mouse model were observed to have achieved better in vivo tumor inhibitory activities than ADC-1IR, ADC-1KR, ADC-1JR, and ADC-1LR.

1. A drug conjugate of formula II or a pharmaceutically acceptable salt thereof, 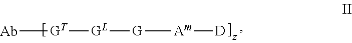
wherein
Ab is an antibody or an antigen-binding fragment thereof,
G^{T} is a group connected to an antibody or an antigen-binding fragment thereof,
G^{L} is a single bond or a linking group; 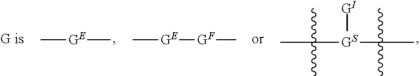
wherein G^{E} is connected to G^{L};
each G^{E} is independently a polypeptide residue consisting of 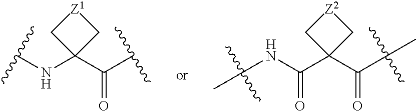
and t amino acid residues, each of the amino acid residues is independently a natural amino acid residue or a non-natural amino acid residue, and t is an integer of 1-10;
Z¹ and Z² are independently —CH₂— or O;
G^{F} and G^{S} are a self-immolative group; G¹ is 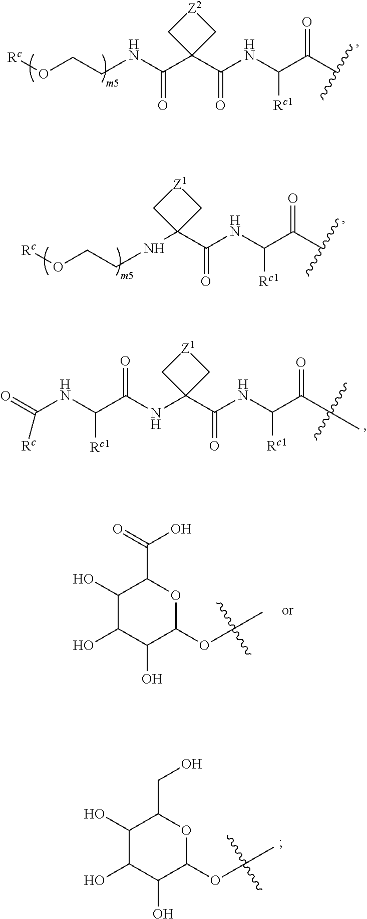
each R^{c} is independently C₁₋₆ alkyl;
each R^{c1} is independently 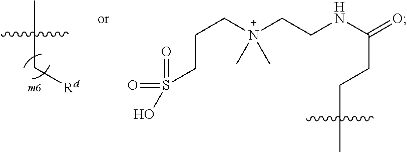
each R^{d} is independently carboxyl, 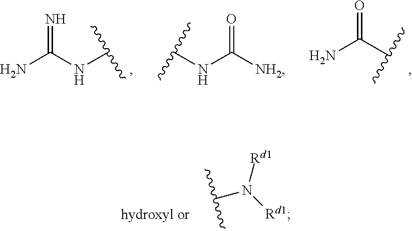
each R^{d1} is independently H or C₁₋₆ alkyl;
m5 and m6 are each independently 1, 2, 3, 4, 5 or 6;
A^{m} is a single bond, a natural amino acid residue or a non-natural amino acid residue;
D is 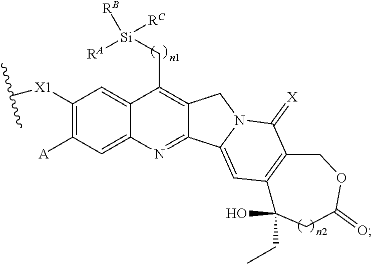
X1 is NH or O;
A is H, halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl;
R^{A}, R^{B} and R^{C} are independently H, halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl;
or, any two of R^{A}, R^{B} and R^{C} together with the silicon atom to which they are attached form a C₅₋₆ cycloalkyl ring or a 5- to 6-membered heterocyclyl ring, and the 5- to 6-membered heterocyclyl optionally further comprises one, two or three types of heteroatoms selected from N, O and S, and the number of the heteroatoms in the 5- to 6-membered heterocyclyl is independently 1, 2 or 3;
X is O or S;
n1 and n2 are independently 0, 1, 2 or 3;
z is an integer or decimal of 1-16;
5- to 10-membered heteroaryl and 3- to 6-membered heterocyclyl each comprise one, two or three types of heteroatoms selected from N, O and S, and the number of the heteroatoms is independently 1, 2 or 3.

2. The drug conjugate of formula II or the pharmaceutically acceptable salt thereof according to claim 1, wherein the drug conjugate satisfies one or more of the following conditions:
(1) Ab is an antibody;
(2) G^{T} is 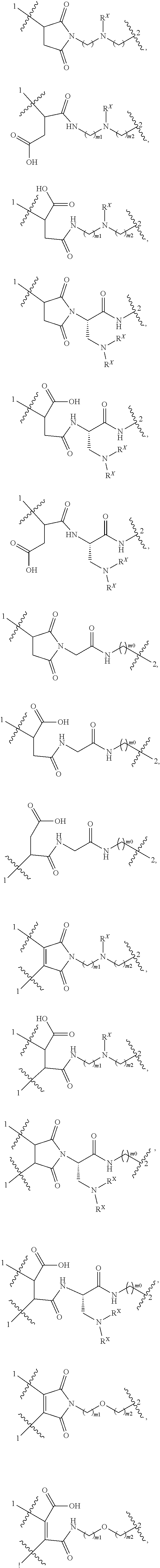 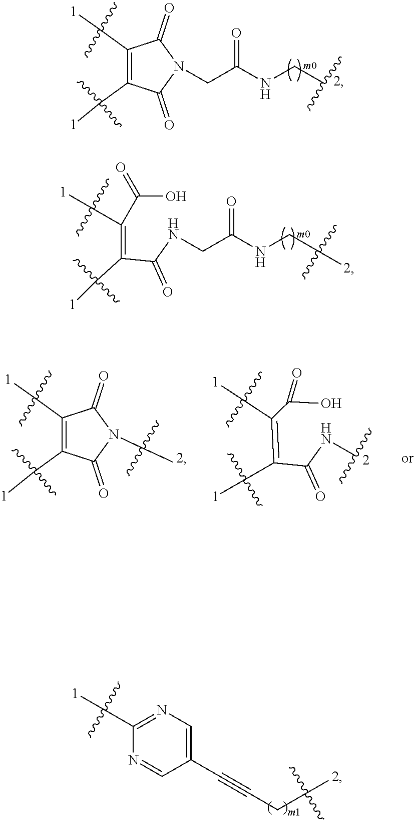
wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl;
(3) G^{L} is -G^{L1}- or -G^{L1}-G^{L2}-, G^{L1} is connected to G^{T};
G^{L1} is 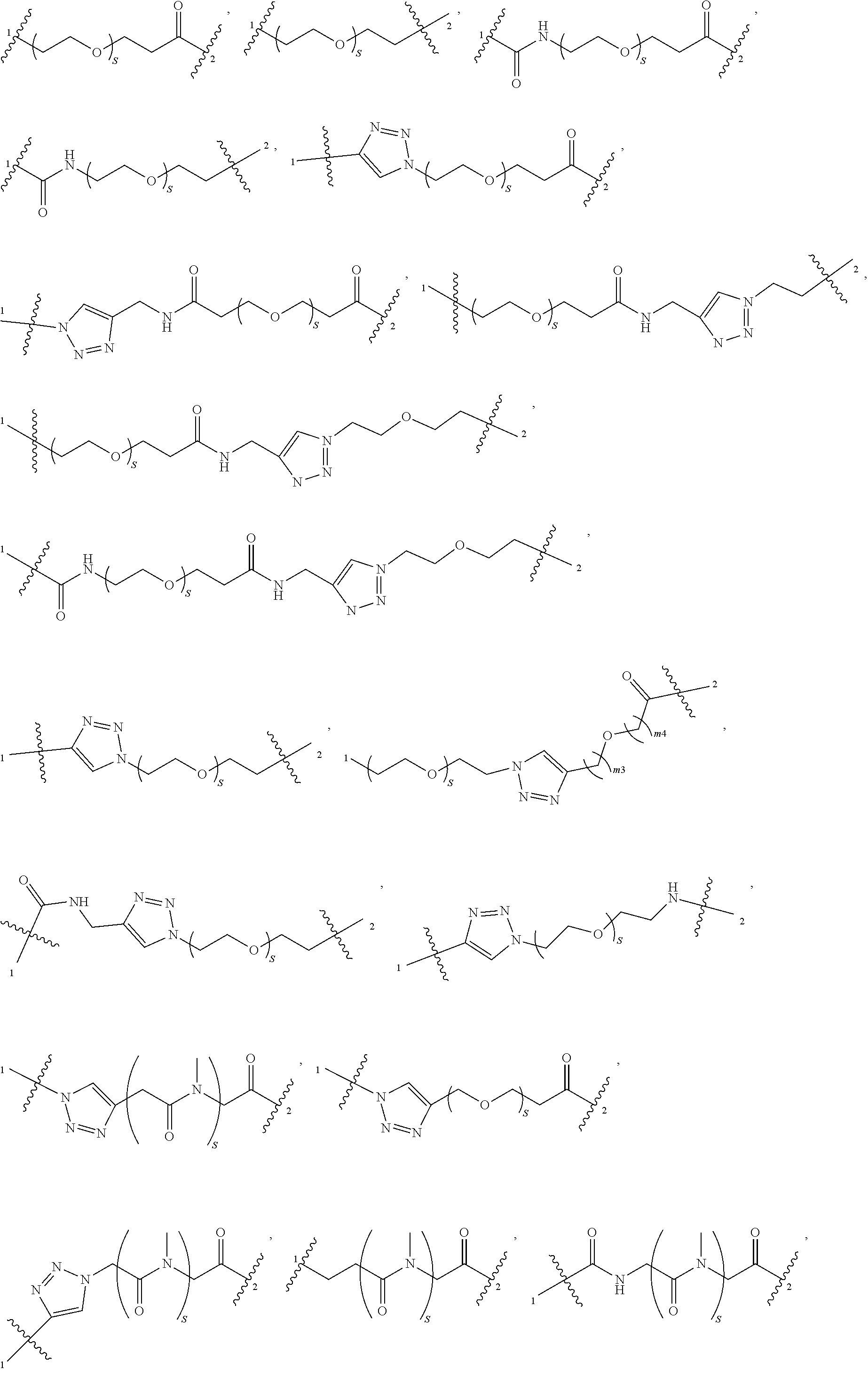 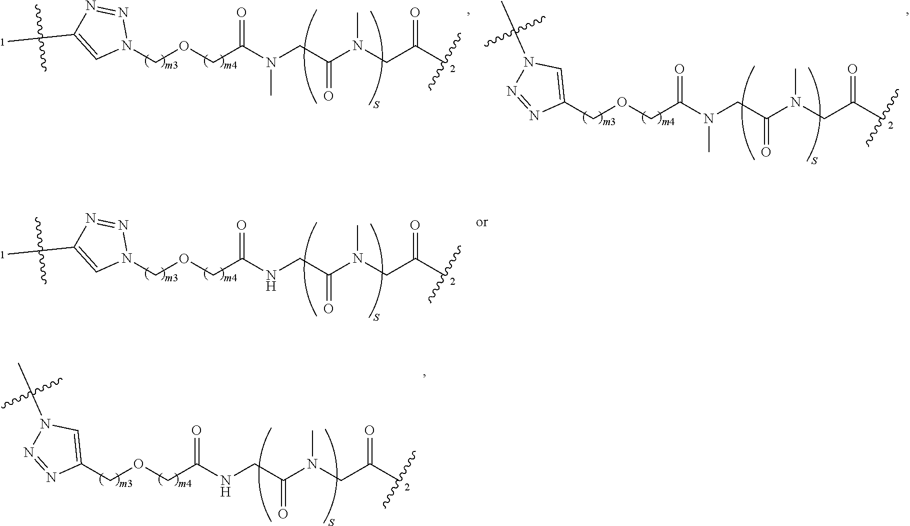
wherein position “1” is connected to G^{T}; position “2” is connected to G^{L2}, G^{E} or G^{S};
m3 and m4 are each independently 1, 2, 3, 4 or 5;
each s is independently an integer from 0-30;
G^{L2} is 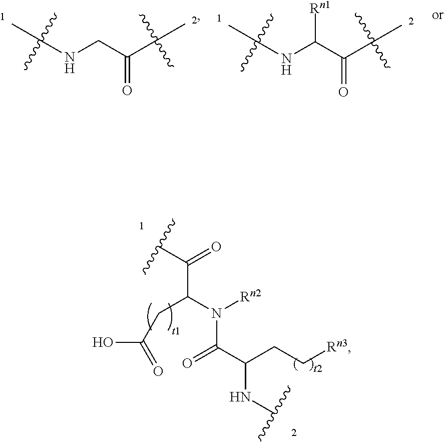
wherein position “1” is connected to G^{L1}; position “2” is connected to G^{E} or G^{S}; t1 and t2 are independently 1, 2, 3 or 4; Rⁿ¹ is C₁₋₄ alkyl, Rⁿ² is H or methyl, and Rⁿ³ is amino, 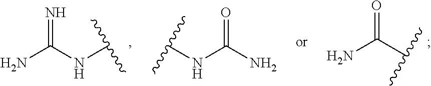
(4) G^{E} is 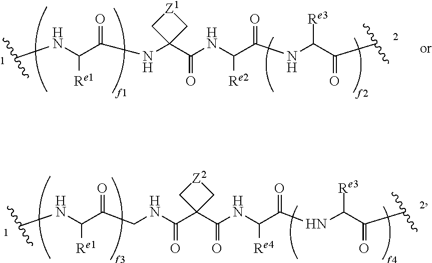
wherein position “1” is connected to G^{L}, and position “2” is connected to G^{F} or D;
Z¹ and Z² are independently —CH₂— or O;
R^{e1}, R^{e2}, R^{e3} and R^{e4} are independently H or —(CH₂)_{f5}—R^{ee};
R^{ee} is —OH, —COOH, —NH₂, —NR^{ee-1}R^{ee-2}, 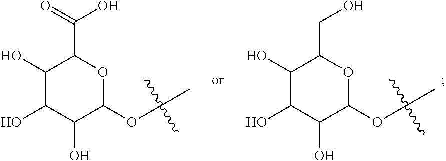 [SMILES: *OC1OC(C(=O)O)C(O)C(O)C1O ; *OC1OC(CO)C(O)C(O)C1O]
R^{ee-1} and R^{ee-2} are independently methyl, ethyl, n-propyl, isopropyl, n-butyl, isobutyl, sec-butyl or tert-butyl;
f1 and f2 are independently 1, 2, 3 or 4; f3 and f4 are independently 0, 1, 2, 3 or 4;
f5 is 1, 2, 3, 4, 5 or 6;
(5) G^{F} is 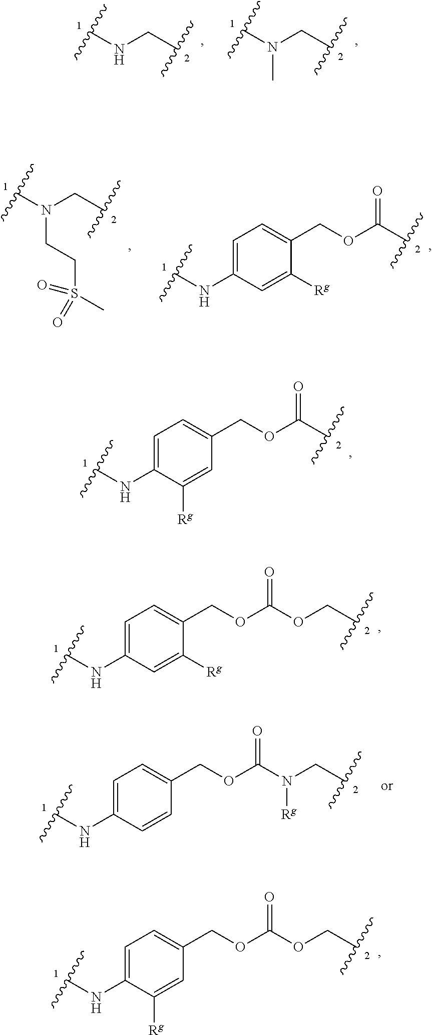
wherein position “1” is connected to G^{E}, and position “2” is connected to A^{m};
each R^{g} is independently H, 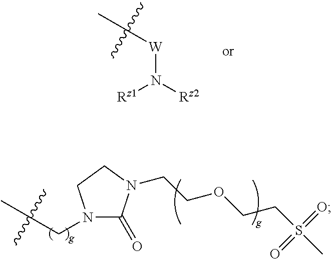
W is independently C₁₋₆ alkylene or C₂₋₆ alkynylene;
R^{z1} and R^{z2} are independently 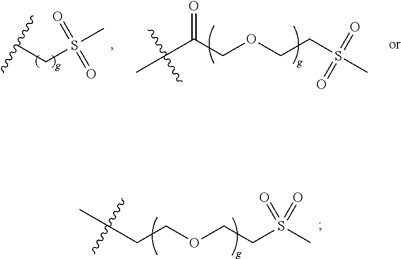
each g is independently 1, 2, 3, 4, 5 or 6;
(6) G^{S} is 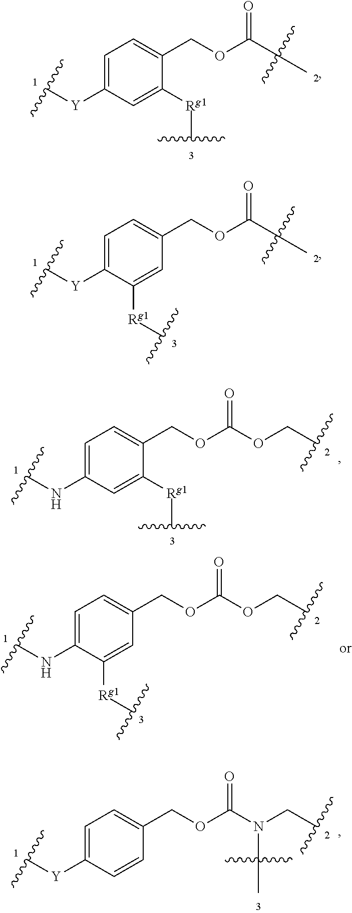
each R^{g1} is independently 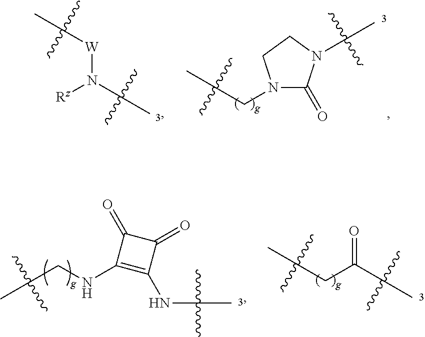
or —NH—³;
wherein position “1” is connected to G¹, position “2” is connected to A^{m}, and position “3” is connected to G^{L};
each Y is independently NH or a single bond;
each W is independently C₁₋₆ alkylene or C₂₋₆ alkynylene;
each R^{z} is independently 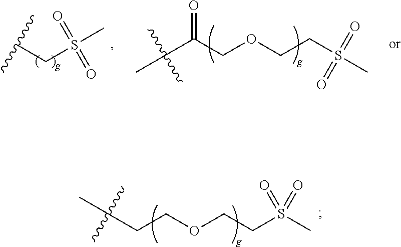
each g is independently 1, 2, 3, 4, 5 or 6;
(7) A^{m} is a single bond or 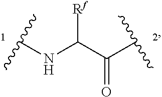
R^{f} is H, C₁₋₆ alkyl or —(CH₂)ₙ—R^{f1}, R^{f1} is —NH₂ or —NR^{ee-1}R^{ee-2}, n is 1, 2, 3, 4, 5 or 6, R^{ee-1} and R^{ee-2} are independently methyl, ethyl, n-propyl, isopropyl, n-butyl, isobutyl, sec-butyl or tert-butyl, wherein position “1” is connected to G^{E}, G^{F} or G^{S}, and position “2” is connected to D.

3. The drug conjugate of formula II or the pharmaceutically acceptable salt thereof according to claim 2, wherein the drug conjugate satisfies one or more of the following conditions:
(1) Ab is an antibody selected from one or more of anti-Her2 antibodies, anti-folate receptor α antibodies, anti-MUC1 antibodies and anti-human IgG1 antibodies; for example, an antibody selected from one or two of anti-Her2 antibodies and anti-MUC1 antibodies; for another example, the Ab is Trastuzumab antibody or a variant thereof, v30384 antibody or a variant thereof, FG1 antibody or a variant thereof, Gatipotuzumab antibody or a variant thereof, 186D antibody, FG1-Tsc antibody or a variant thereof or FG1-Tsc thiomab or a variant thereof,
the variant of the Trastuzumab antibody has at least 90%, at least 95%, at least 96%, at least 97%, at least 98%, at least 99%, at least 99.5% or at least 99.9% homology to the sequence of the Trastuzumab antibody, and the variant of the Trastuzumab antibody has the same CDR sequences as the Trastuzumab antibody;
the heavy chain of the v30384 antibody has an amino acid sequence as shown in SEQ ID NO: 3, and an amino acid sequence as shown in SEQ ID NO: 4, and the light chain of the v30384 antibody has an amino acid sequence as shown in SEQ ID NO: 5; the variant of the v30384 antibody has at least 90%, at least 95%, at least 96%, at least 97%, at least 98%, at least 99%, at least 99.5% or at least 99.9% homology to the sequence of the v30384 antibody, and the variant of the v30384 antibody has the same CDR sequences as the v30384 antibody;
the heavy chain of the FG1 antibody has an amino acid sequence as shown in SEQ ID NO: 6, and the light chain of the FG1 antibody has an amino acid sequence as shown in SEQ ID NO: 7; the variant of the FG1 antibody has at least 90%, at least 95%, at least 96%, at least 97%, at least 98%, at least 99%, at least 99.5% or at least 99.9% homology to the sequence of the FG1 antibody; the variant of the FG1 antibody has the same CDR sequences as the FG1 antibody;
the heavy chain of the Gatipotuzumab antibody has an amino acid sequence as shown in SEQ ID NO: 8, and the light chain of the Gatipotuzumab antibody has an amino acid sequence as shown in SEQ ID NO: 9; the variant of the Gatipotuzumab antibody has at least 90%, at least 95%, at least 96%, at least 97%, at least 98%, at least 99%, at least 99.5% or at least 99.9% homology to the sequence of the Gatipotuzumab antibody, and the variant of the Gatipotuzumab antibody has the same CDR sequences as the Gatipotuzumab antibody;
the heavy chain of the 186D antibody has the amino acid sequence as shown in SEQ ID NO: 10, and the light chain of the 186D antibody has the amino acid sequence as shown in SEQ ID NO: 11; the variant of the 186D antibody has at least 90%, at least 95%, at least 96%, at least 97%, at least 98%, at least 99%, at least 99.5% or at least 99.9% homology to the sequence of the 186D antibody; the variant of the 186D antibody has the same CDR sequences as the 186D antibody;
the heavy chain of the FG1-Tsc antibody has an amino acid sequence as shown in SEQ ID NO: 12, and the light chain of the FG1-Tsc antibody has an amino acid sequence as shown in SEQ ID NO: 13; the variant of the FG1-Tsc antibody has at least 90%, at least 95%, at least 96%, at least 97%, at least 98%, at least 99%, at least 99.5% or at least 99.9% homology to the sequence of the FG1-Tsc antibody, and the variant of the FG1-Tsc antibody has the same CDR sequences as the FG1-Tsc antibody;
the heavy chain of the FG1-Tsc thiomab antibody has an amino acid sequence as shown in SEQ ID NO: 20, and the light chain of the FG1-Tsc thiomab antibody has an amino acid sequence as shown in SEQ ID NO: 21; the variant of the FG1-Tsc thiomab antibody has at least 90%, at least 95%, at least 96%, at least 97%, at least 98%, at least 99%, at least 99.5% or at least 99.9% homology to the sequence of the FG1-Tsc thiomab antibody, and the variant of the FG1-Tsc thiomab antibody has the same CDR sequences as the FG1-Tsc thiomab antibody;
(2) G^{T} is selected from Scheme 1, Scheme 2 and Scheme 3: 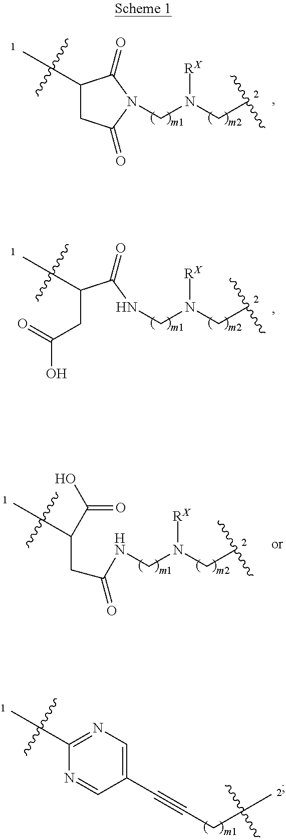 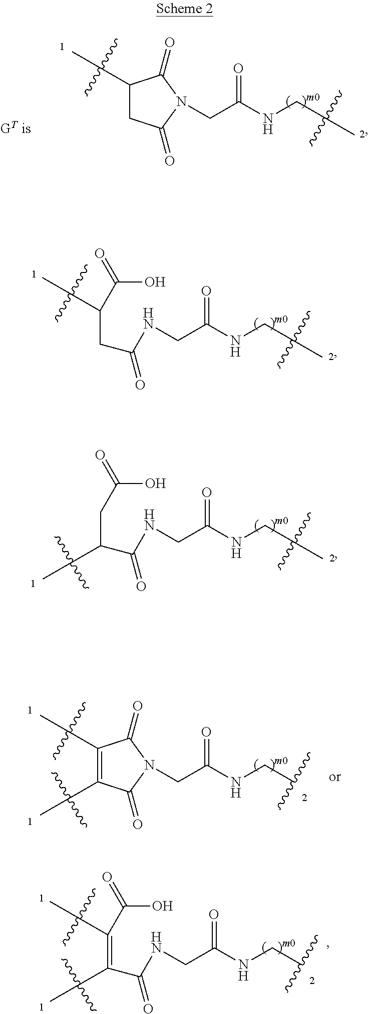
and m0 is 1, 2, 3, 4 or 5;
(3) each R^{x} is independently H, methyl, ethyl, n-propyl, isopropyl, n-butyl, isobutyl, sec-butyl or tert-butyl, for example, H or methyl;
(4) G^{L1} is 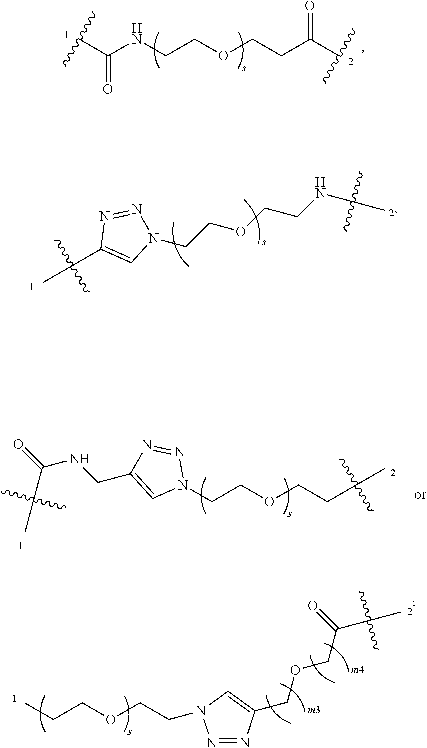
(5) G^{L2} is 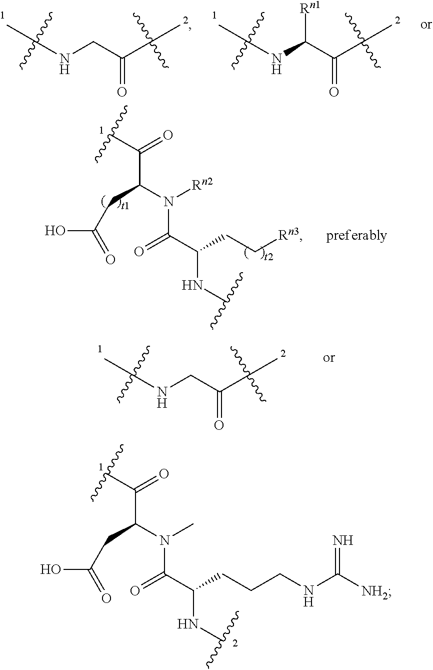
(6) each s is independently 1, 2, 3, 4, 5 or 6;
(7) G^{E} is selected from Scheme 1 and Scheme 2:
Scheme 1: G^{E} is 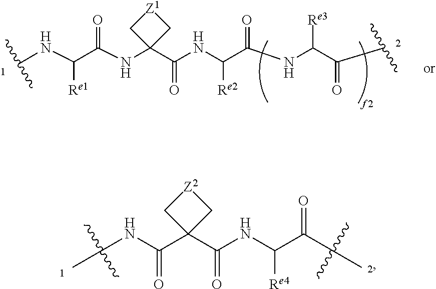
f2 is 1 or 2, for example 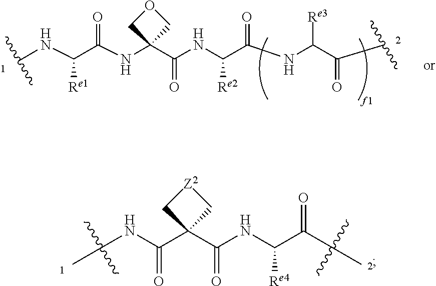
Scheme 2: G^{E} is 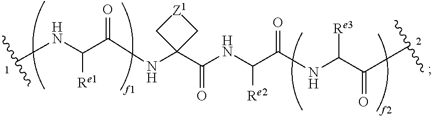
for example 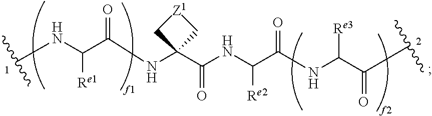
(8) R^{e1} is —(CH₂)_{f5}—COOH, for example, 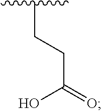 [SMILES: *CCC(=O)O]
(9) R^{e2} is —(CH₂)_{f5}—R^{ee}, R^{ee} is —OH, —NH₂, —NR^{ee-1}R^{ee-2} or 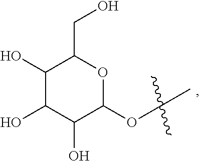 [SMILES: *OC1OC(CO)C(O)C(O)C1O]
for example, R^{e2} is 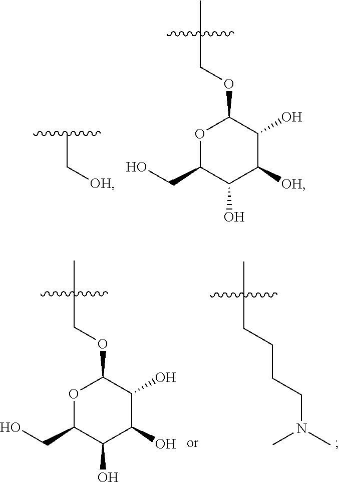
(10) R^{e3} is H;
(11) R^{e4} is —(CH₂)_{f5}—R^{ee}, R^{ee} is —OH, —NH₂ or —NR^{ee-1}R^{ee-2}, for example, 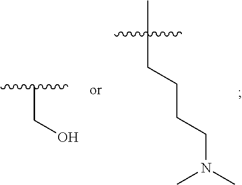
(12) G^{F} is 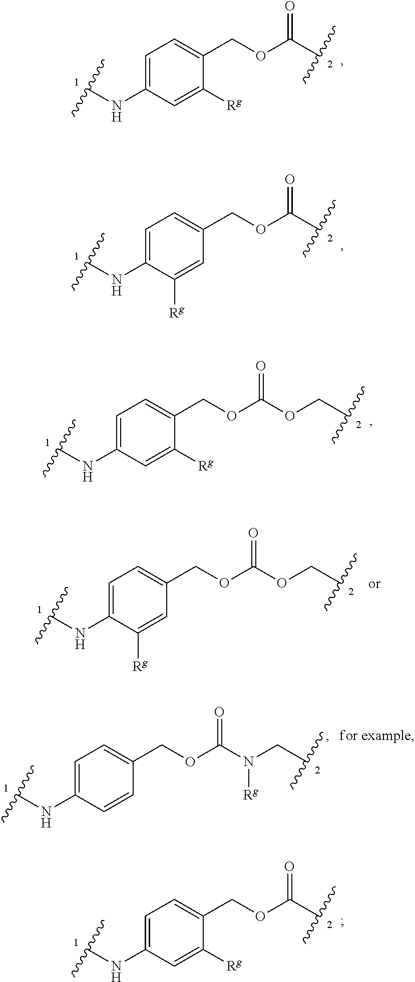
(13) G^{S} is 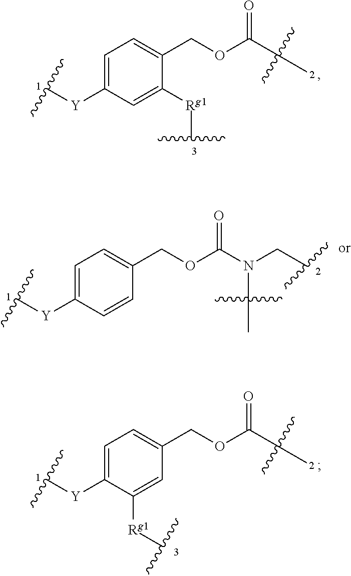
(14) each W is independently methylene, 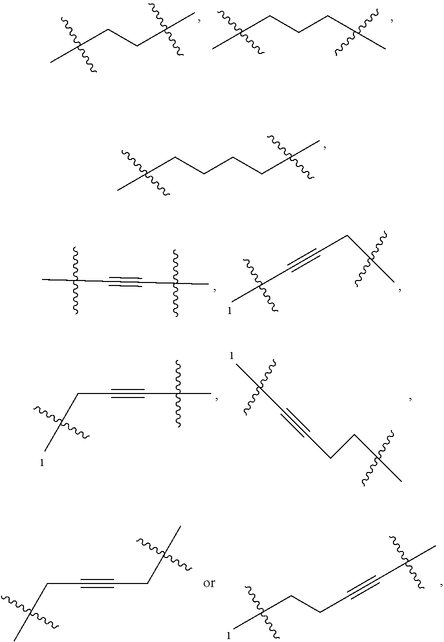
wherein position “1” is connected to a benzene ring;
(15) R^{z} is 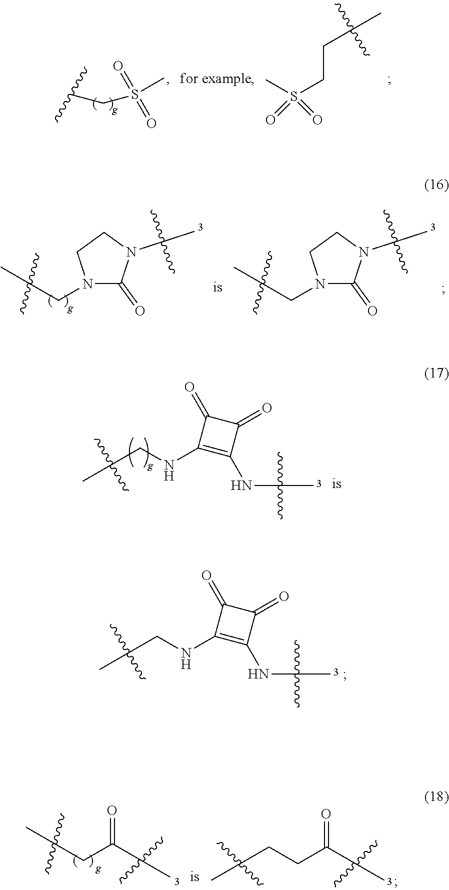
(19) G^{I} is 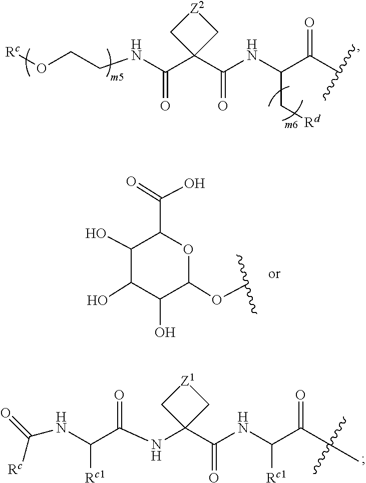
for example, 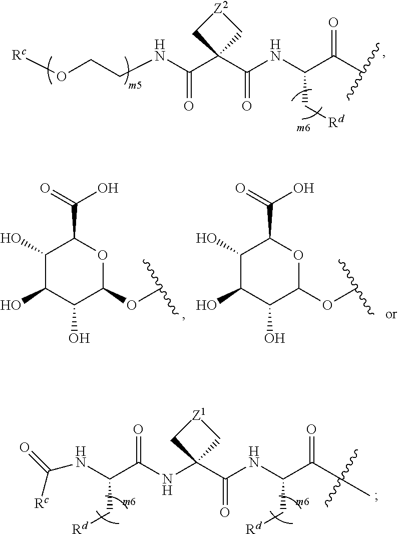
(20) R^{c} and R^{d1} are each independently methyl, ethyl, n-propyl, isopropyl, n-butyl, isobutyl, sec-butyl or tert-butyl, for example, methyl;
(21) A is H, F, Cl, Br or I, for example, H or F;
(22) R^{A}, R^{B} and R^{C} are independently C₁₋₆ alkyl, for example, methyl, ethyl, n-propyl, isopropyl, n-butyl, isobutyl, sec-butyl or tert-butyl, for another example, methyl;
(23) X is O;
(24) n1 is 2;
(25) n2 is 0;
(26) z is an integer or decimal of 1-8, for example, an integer or decimal of 3-5, or an integer or decimal of 6-8.

4. The drug conjugate of formula II or the pharmaceutically acceptable salt thereof according to claim 1, wherein the drug conjugate satisfies one or more of the following conditions:
(1) G^{T} is 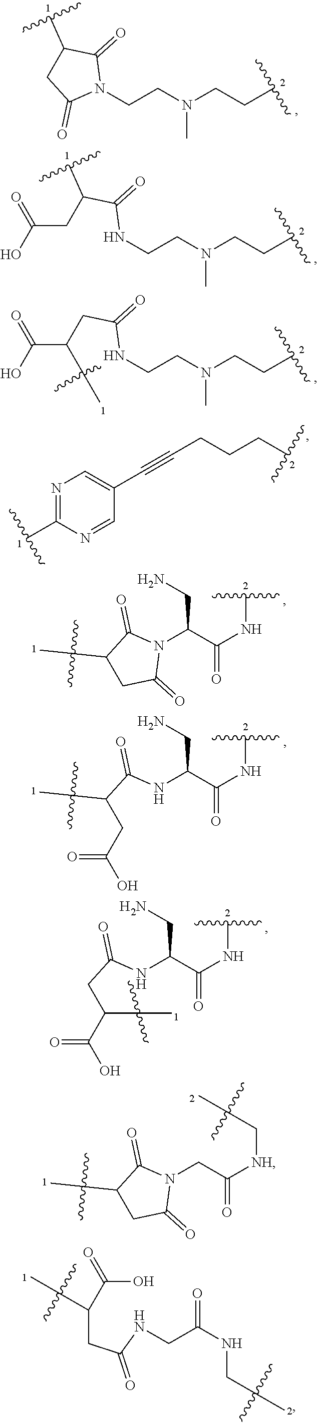 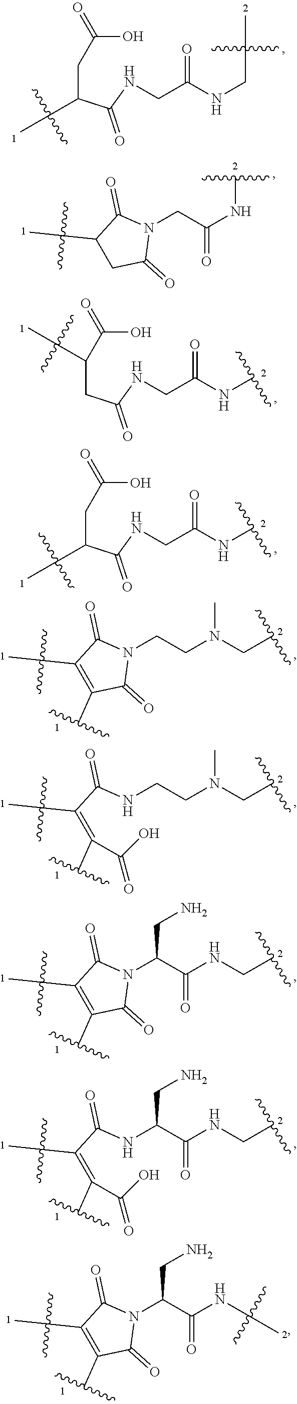 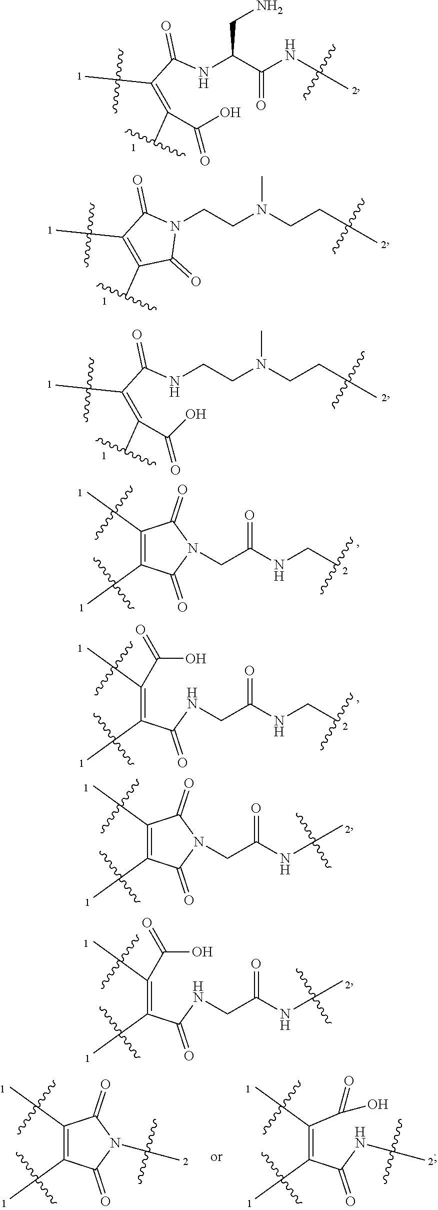
(2) G^{L1} is 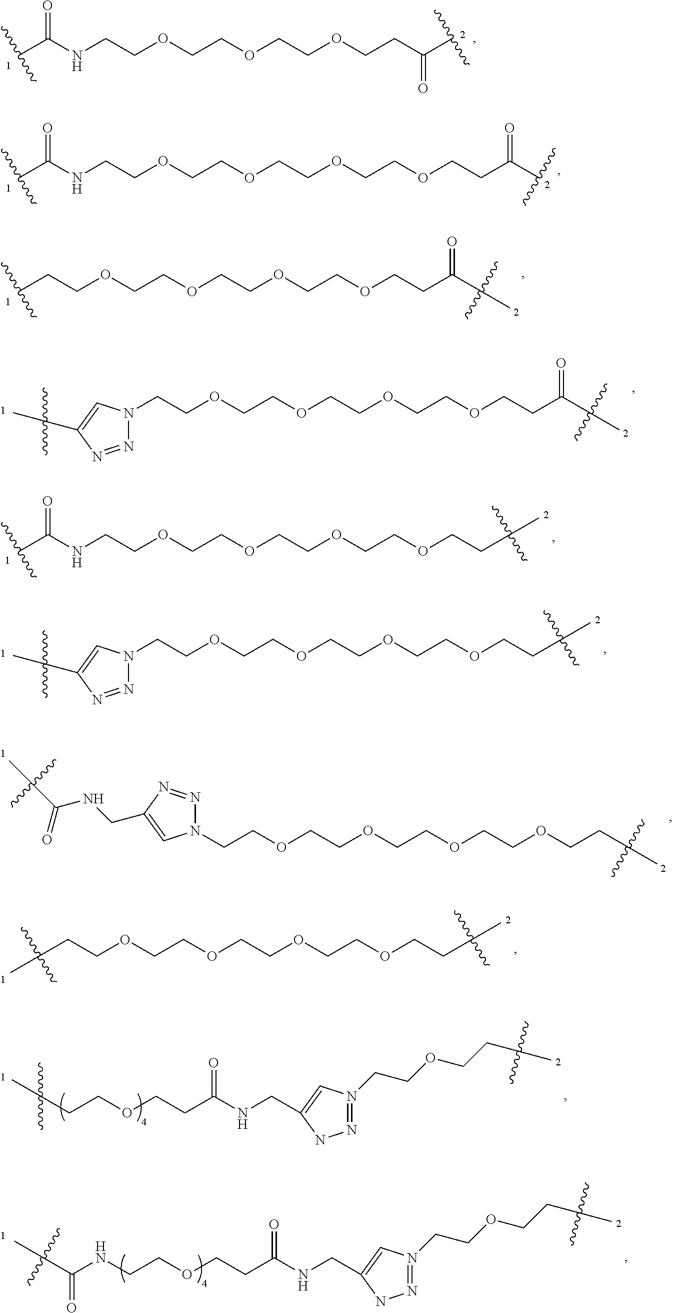 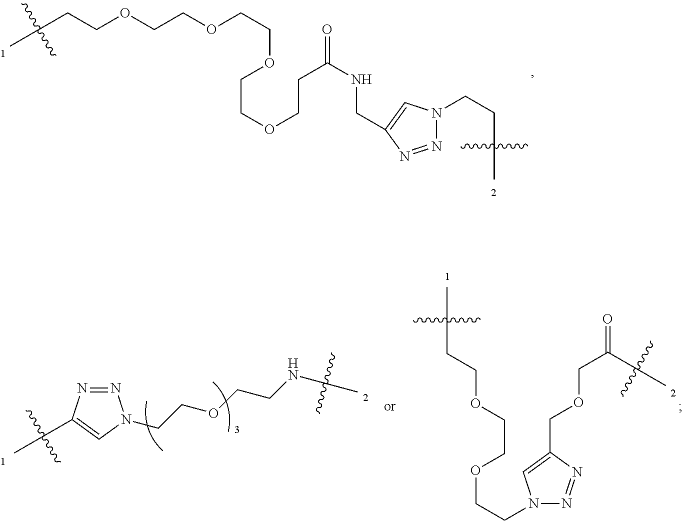
(3) G^{L2} is 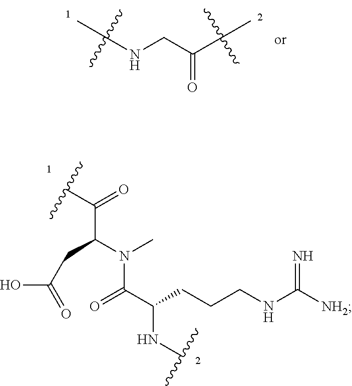 [SMILES: *NCC(*)=O ; *N[C@@H](CCCNC(=N)N)C(=O)N(C)[C@@H](CC(=O)O)C(*)=O]
(4) G^{E} is 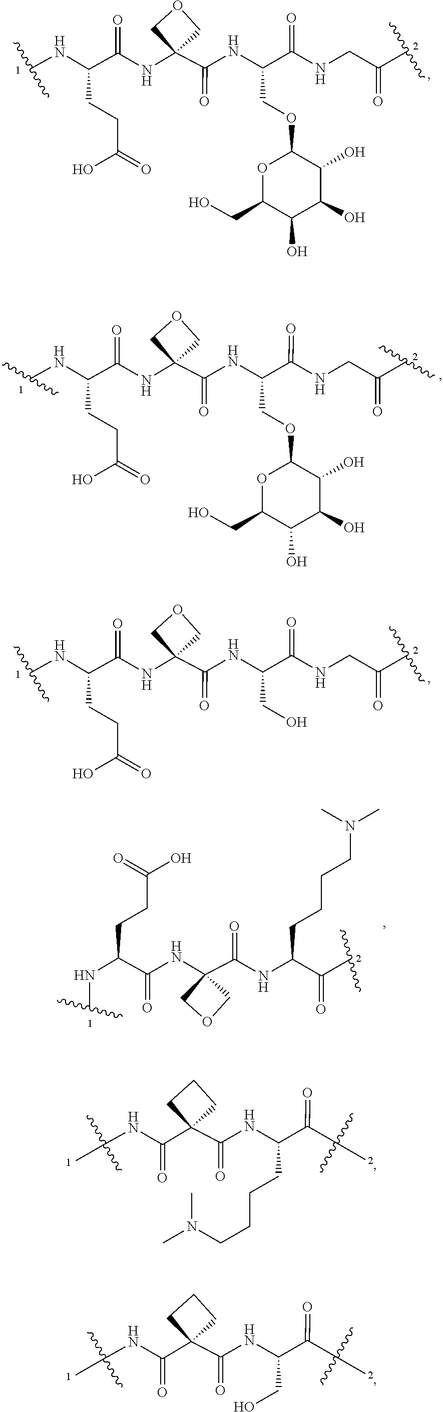 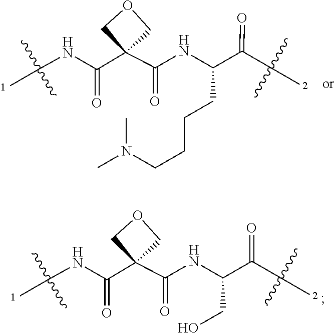
(5) G^{F} is 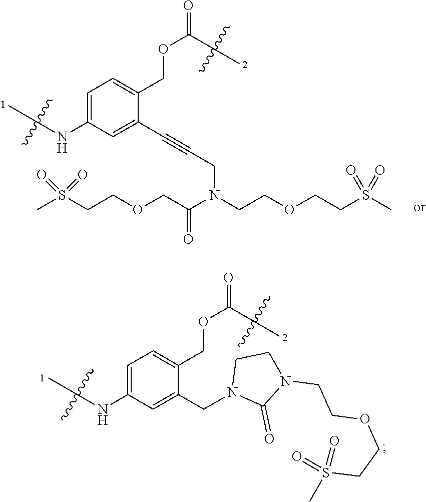 [SMILES: *Nc1ccc(COC(*)=O)c(C#CCN(CCOCCS(C)(=O)=O)C(=O)COCCS(C)(=O)=O)c1 ; *Nc1ccc(COC(*)=O)c(CN2CCN(CCOCCS(C)(=O)=O)C2=O)c1]
(6) G^{S} is 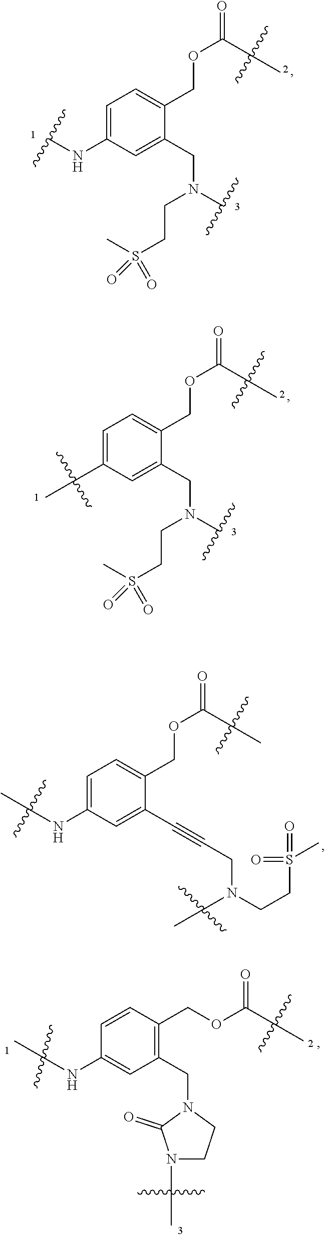 [SMILES: *Nc1ccc(COC(*)=O)c(CN(*)CCS(C)(=O)=O)c1 ; *C(=O)OCc1ccc(*)cc1CN(*)CCS(C)(=O)=O ; *Nc1ccc(COC(*)=O)c(C#CCN(*)CCS(C)(=O)=O)c1 ; *Nc1ccc(COC(*)=O)c(CN2CCN(*)C2=O)c1] 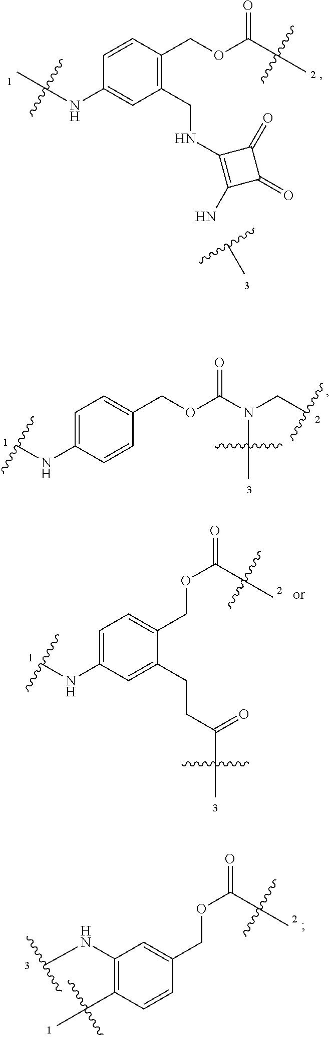
(7) G^{I} is 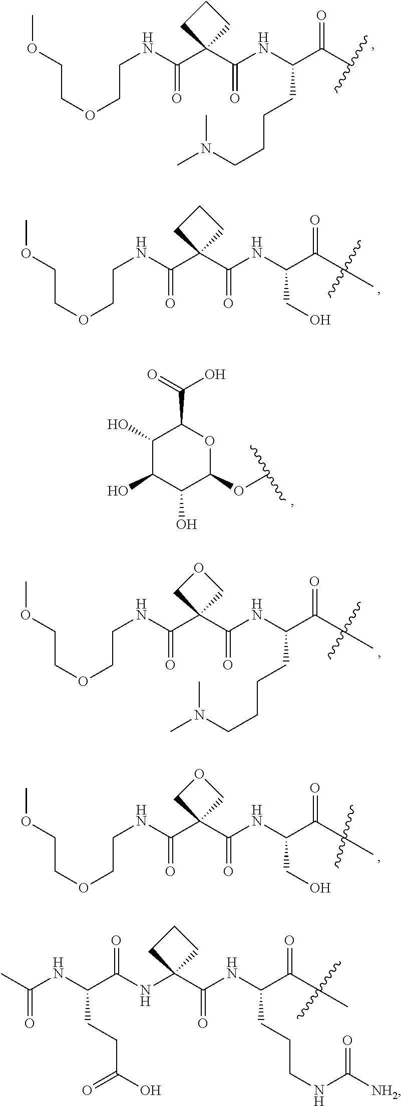 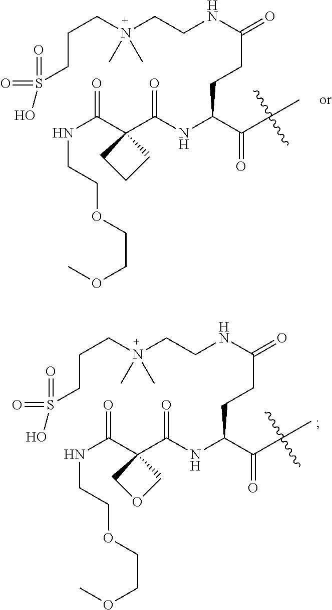
(8) A^{m} is a single bond, 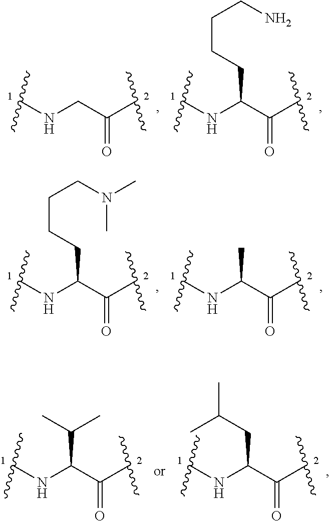
wherein position “1” is connected to G^{E}, G^{F} or G^{S}, and position “2” is connected to D;
(9) D is

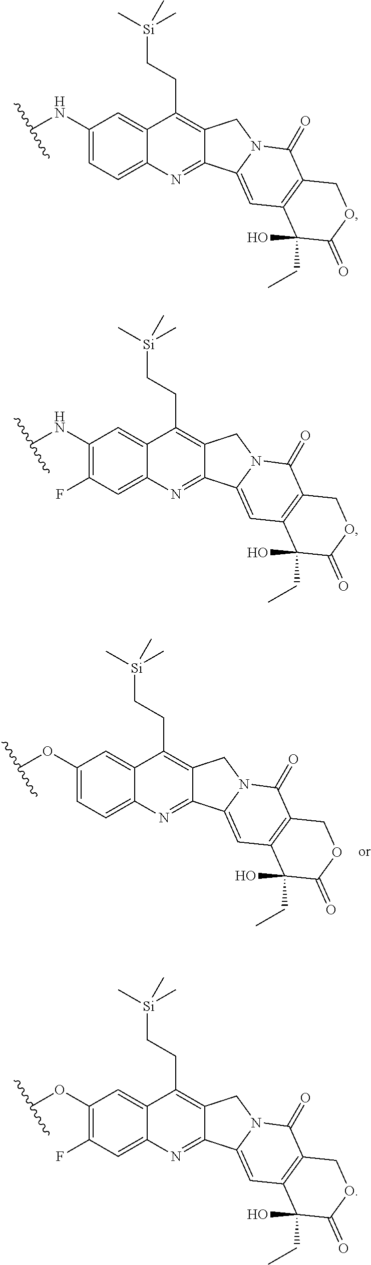 [SMILES: *Nc1ccc2nc3c(c(CC[Si](C)(C)C)c2c1)Cn1c-3cc2c(c1=O)COC(=O)[C@]2(O)CC ; *Nc1cc2c(CC[Si](C)(C)C)c3c(nc2cc1F)-c1cc2c(c(=O)n1C3)COC(=O)[C@]2(O)CC ; *Oc1ccc2nc3c(c(CC[Si](C)(C)C)c2c1)Cn1c-3cc2c(c1=O)COC(=O)[C@]2(O)CC ; *Oc1cc2c(CC[Si](C)(C)C)c3c(nc2cc1F)-c1cc2c(c(=O)n1C3)COC(=O)[C@]2(O)CC]

5. The drug conjugate of formula II or the pharmaceutically acceptable salt thereof according to claim 2, wherein
-G^{T}-G^{L1}- is

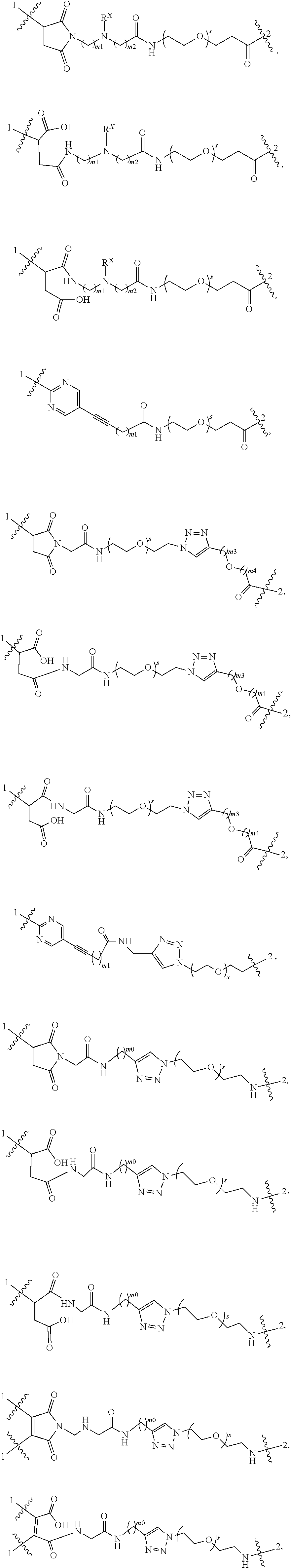

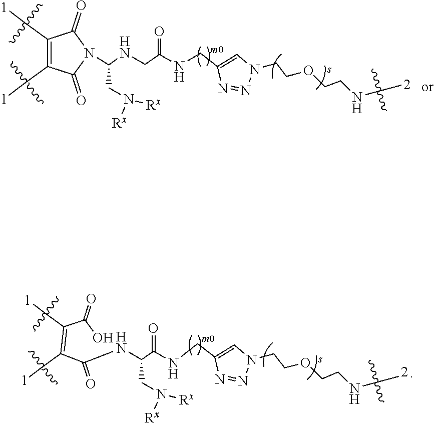

6. The drug conjugate of formula II or the pharmaceutically acceptable salt thereof according to claim 1, wherein the drug conjugate of formula II is in Scheme 1, Scheme 2 or Scheme 3:
Scheme 1: the drug conjugate of formula II is a drug conjugate of formula IIa, 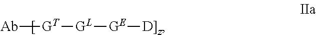
Ab is an antibody selected from one or more of anti-Her2 antibodies, anti-folate receptor α antibodies, anti-MUC1 antibodies and anti-human IgG1 antibodies;
G^{T} is 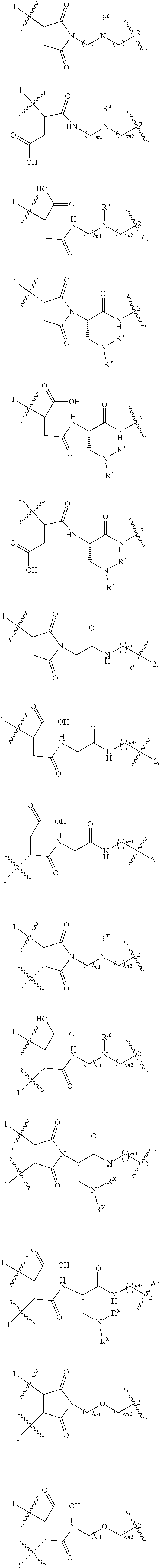 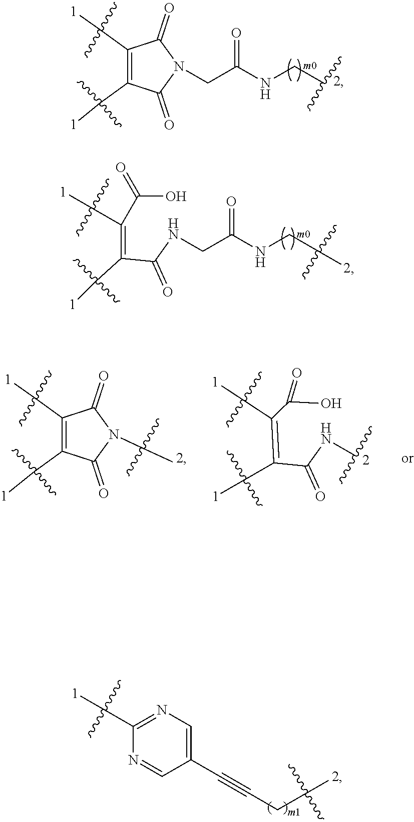
wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl;
G^{L} is 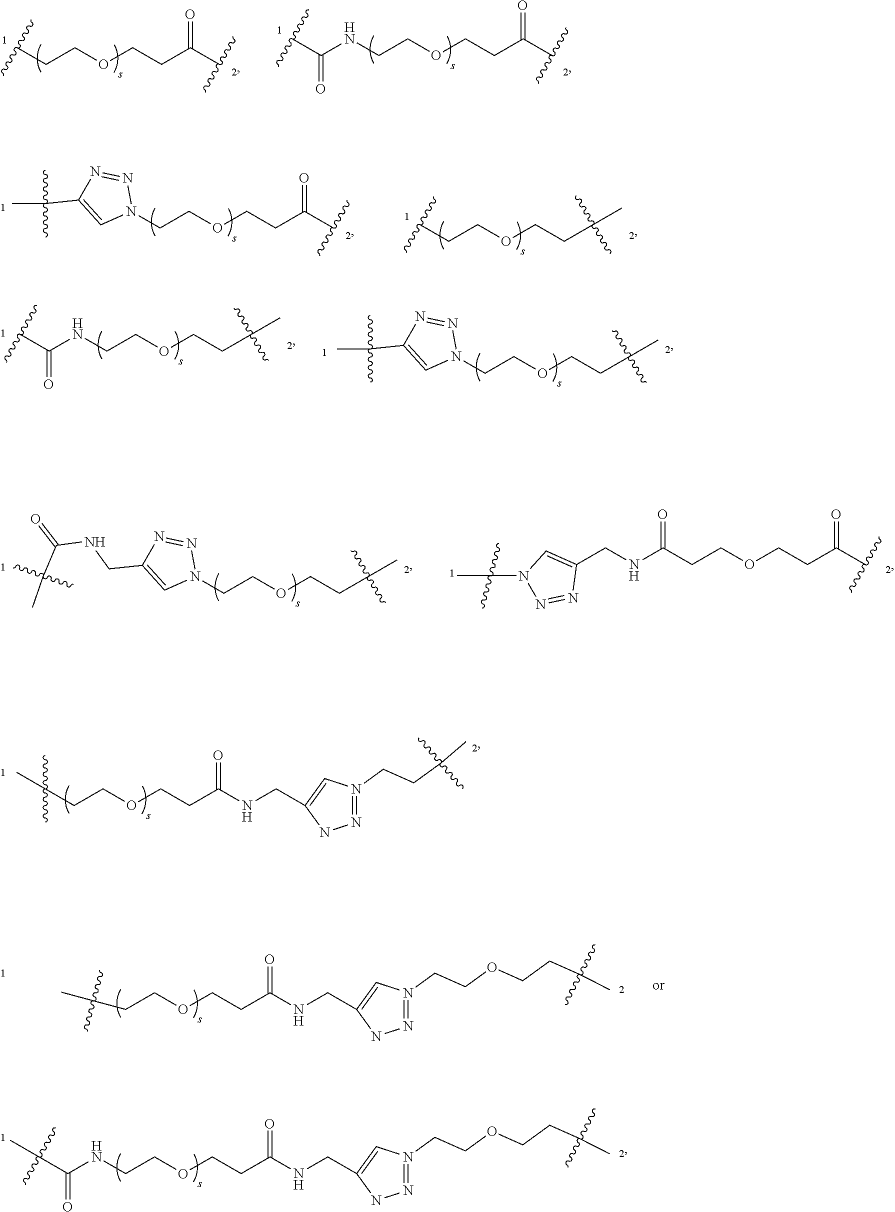
wherein position “1” is connected to G^{T}; position “2” is connected to G^{E};
each s is independently 1, 2, 3, 4, 5 or 6;
G^{E} is 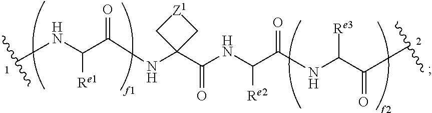
Z¹ is —CH₂— or O; R^{e1}, R^{e2} and R^{e3} are independently H or —(CH₂)_{f2}—R^{ee}, R^{ee} is —OH, —COOH, —NH₂, —NR^{ee-1}R^{ee-2}, 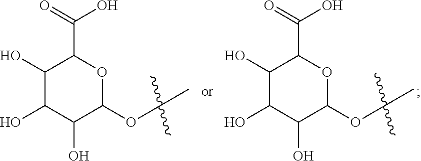 [SMILES: *OC1OC(C(=O)O)C(O)C(O)C1O ; *OC1OC(C(=O)O)C(O)C(O)C1O]
R^{ee-1} and R^{ee-2} are independently methyl, ethyl, n-propyl, isopropyl, n-butyl, isobutyl, sec-butyl or tert-butyl, f1 and f2 are independently 1, 2, 3 or 4;
D is 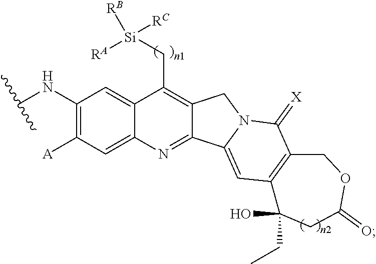
A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8;
Scheme 2: the drug conjugate of formula II is a drug conjugate of formula IIa, 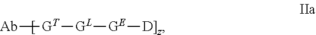
Ab is a Trastuzumab antibody, an FG1 antibody or an FG1-Tsc thiomab antibody;
G^{T} is 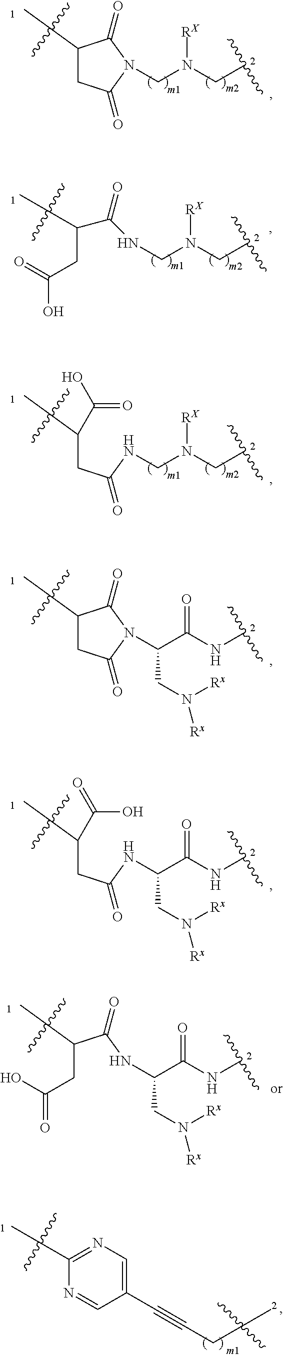
wherein position “1” is connected to Ab, and position “2” is connected to G^{L};
m1 and m2 are each independently 1, 2, 3, 4 or 5;
each R^{x} is independently H or C₁₋₆ alkyl;
G^{L} is 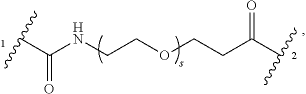
wherein position “1” is connected to G^{T}; position “2” is connected to G^{E};
s is 1, 2, 3, 5 or 6;
G^{E} is 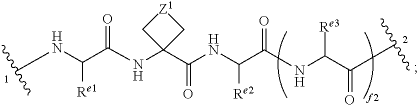
Z¹ is —CH₂— or O; R^{e1}, R^{e2} and R^{e3} are independently H or —(CH₂)_{f2}—R^{ee}, R^{ee} is —OH, —COOH, —NH₂, —NR^{ee-1}R^{ee-2}, 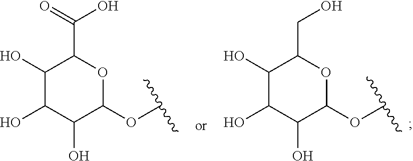 [SMILES: *OC1OC(C(=O)O)C(O)C(O)C1O ; *OC1OC(CO)C(O)C(O)C1O]
R^{ee-1} and R^{ee-2} are independently methyl, ethyl, n-propyl, isopropyl, n-butyl, isobutyl, sec-butyl or tert-butyl, f2 is 1 or 2;
D is 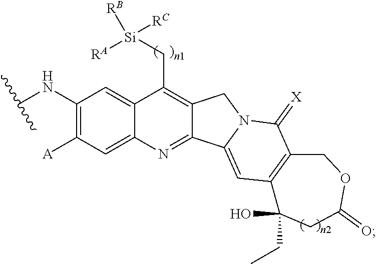
A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8;
Scheme 3: the drug conjugate of formula II is a drug conjugate of formula IIb, 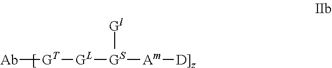
Ab is an antibody selected from one or more of anti-Her2 antibodies, anti-folate receptor α antibodies, anti-MUC1 antibodies and anti-human IgG1 antibodies; 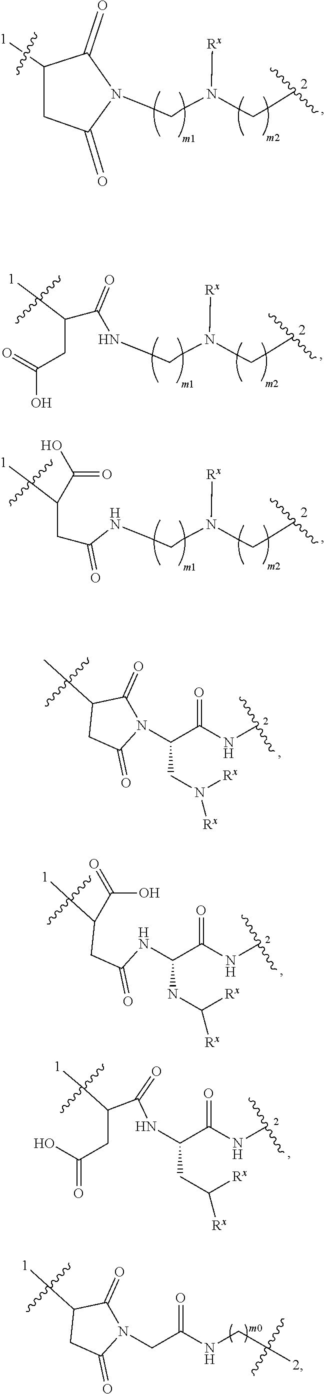 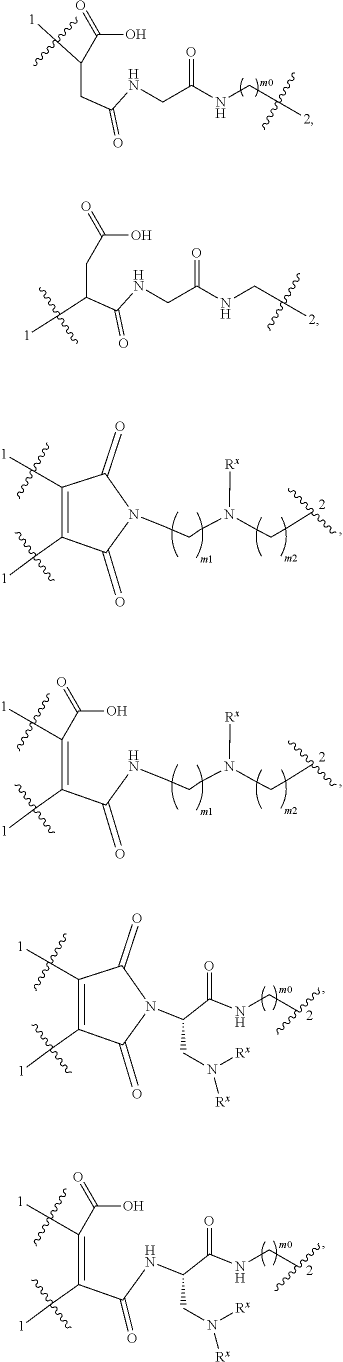 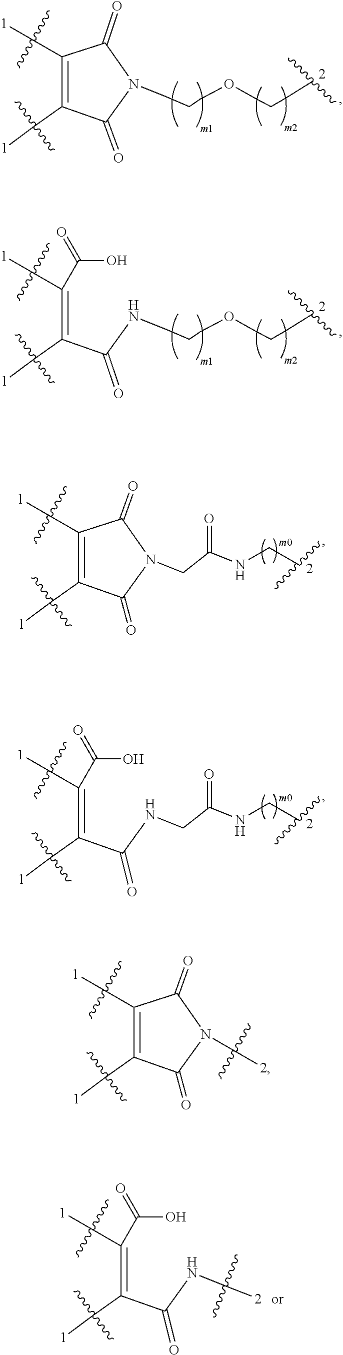 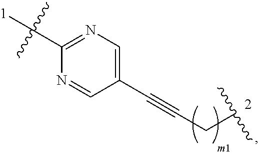
wherein position “1” is connected to Ab, and position “2” is connected to G^{L};
each m0 is independently 0, 1, 2, 3, 4 or 5;
m1 and m2 are each independently 1, 2, 3, 4 or 5;
each R^{x} is independently H or C₁₋₆ alkyl;
G^{L} is 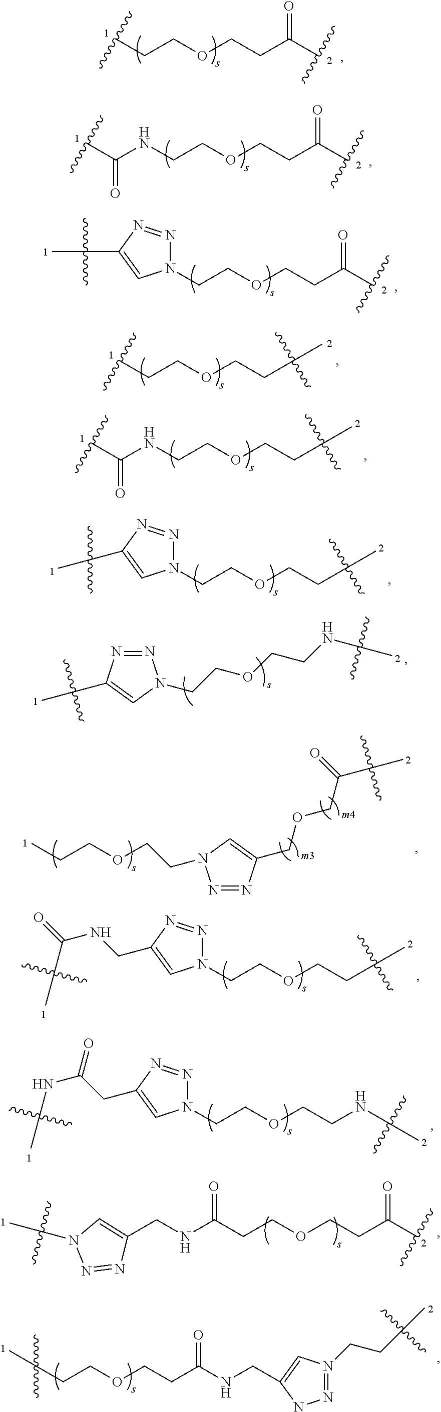 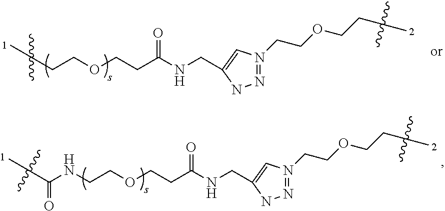
wherein position “1” is connected to G^{T}, and position “2” is connected to G^{L2} or G^{s};
each s is independently 1, 2, 3, 4, 5 or 6;
G^{L2} is 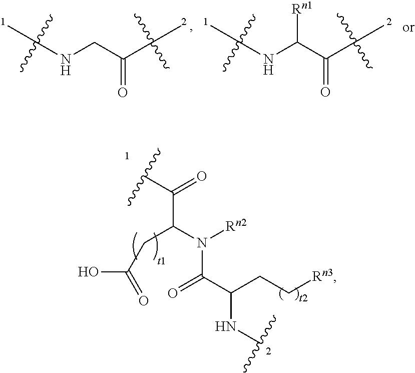
wherein position “1” is connected to G^{L1}; position “2” is connected to G^{E} or G^{S}; t1 and t2 are independently 1, 2, 3 or 4; Rⁿ¹ is C₁₋₄ alkyl, Rⁿ² is H or methyl, and Rⁿ³ is amino, 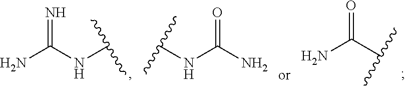
G^{S} is 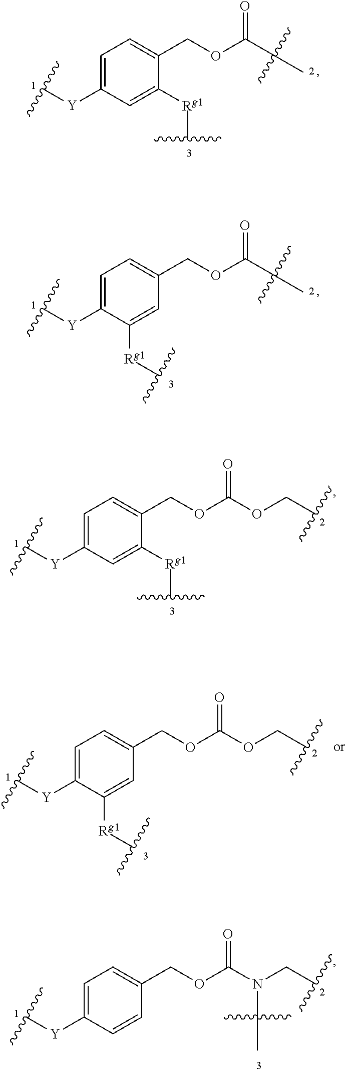
wherein each R^{g1} is independently 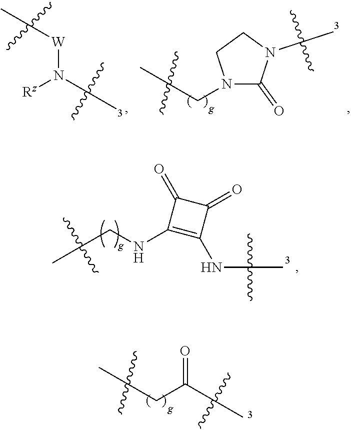
or —NH—³; wherein position “1” is connected to G^{I}, position “2” is connected to A^{m}, and position “3” is connected to G^{L};
each Y is independently NH or a single bond;
each W is independently C₁₋₆ alkylene or C₂₋₆ alkynylene;
each R^{z} is independently 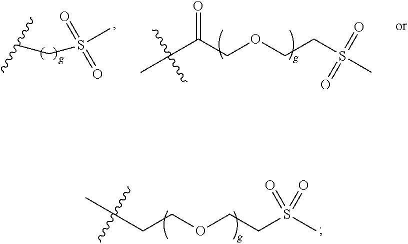
each g is independently 1, 2, 3, 4, 5 or 6;
G^{I} is 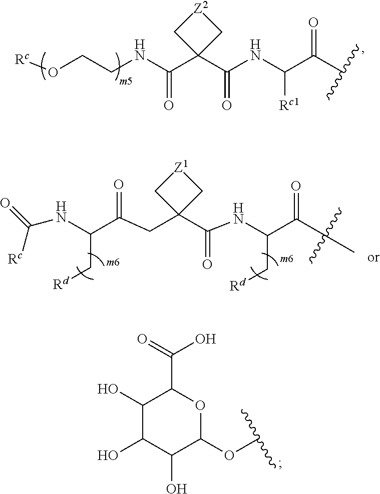
Z² is —CH₂— or O;
R^{c} is C₁₋₆ alkyl;
R^{c1} is 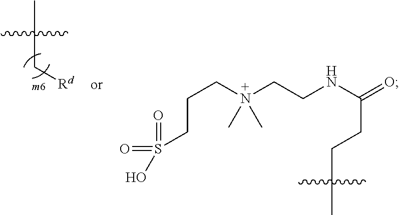
R^{d} is hydroxyl 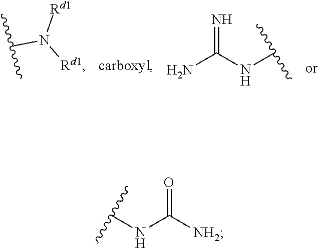
each R^{d1} is independently H or C₁₋₆ alkyl;
m5 and m6 are independently 1, 2, 3, 4, 5 or 6;
A^{m} is a single bond or 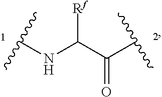
R^{f} is H, C₁₋₆ alkyl or —(CH₂)ₙ—R^{f1}, R^{f1} is —NH₂ or —NR^{ee-1}R^{ee-2} n is 1, 2, 3, 4, 5 or 6, R^{ee-1} and R^{ee-2} are independently methyl, ethyl, n-propyl, isopropyl, n-butyl, isobutyl, sec-butyl or tert-butyl, wherein position “1” is connected to G^{S}, and position “2” is connected to D,
D is 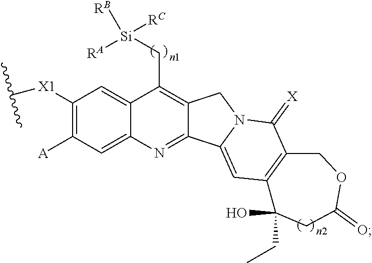
X1 is NH or O;
A is H, F, Cl, Br or I;
R^{A}, R^{B} and R^{C} are independently C₁₋₆ alkyl;
X is O;
n1 and n2 are independently 0, 1, 2 or 3;
z is an integer or decimal of 1-8;
preferably, the drug conjugate of formula IIb is a drug conjugate of formula IIb-1, 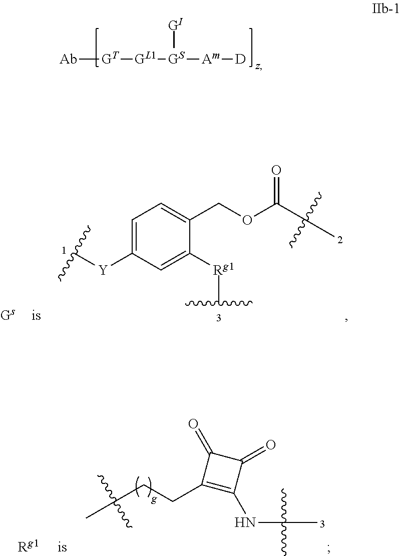
or, the drug conjugate of formula IIb is a drug conjugate of formula IIb-2, 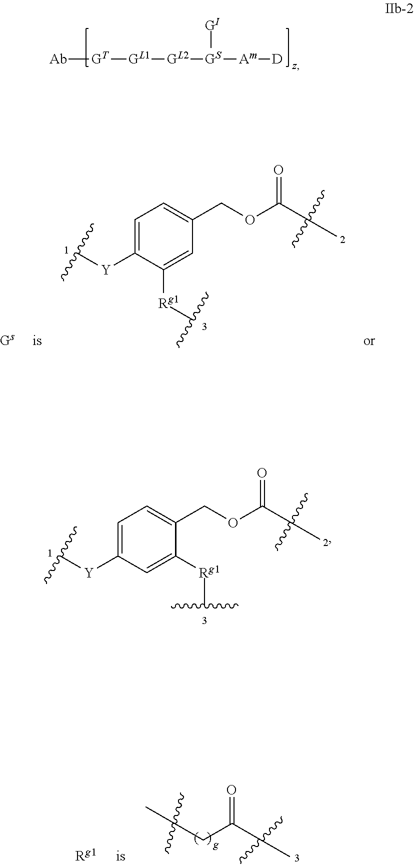
or —NH—³; R^{g1} is —NH—³.

7. The drug conjugate of formula II or the pharmaceutically acceptable salt thereof according to claim 1, wherein the drug conjugate of formula II is any one of the following drug conjugates or hydrolysates thereof: 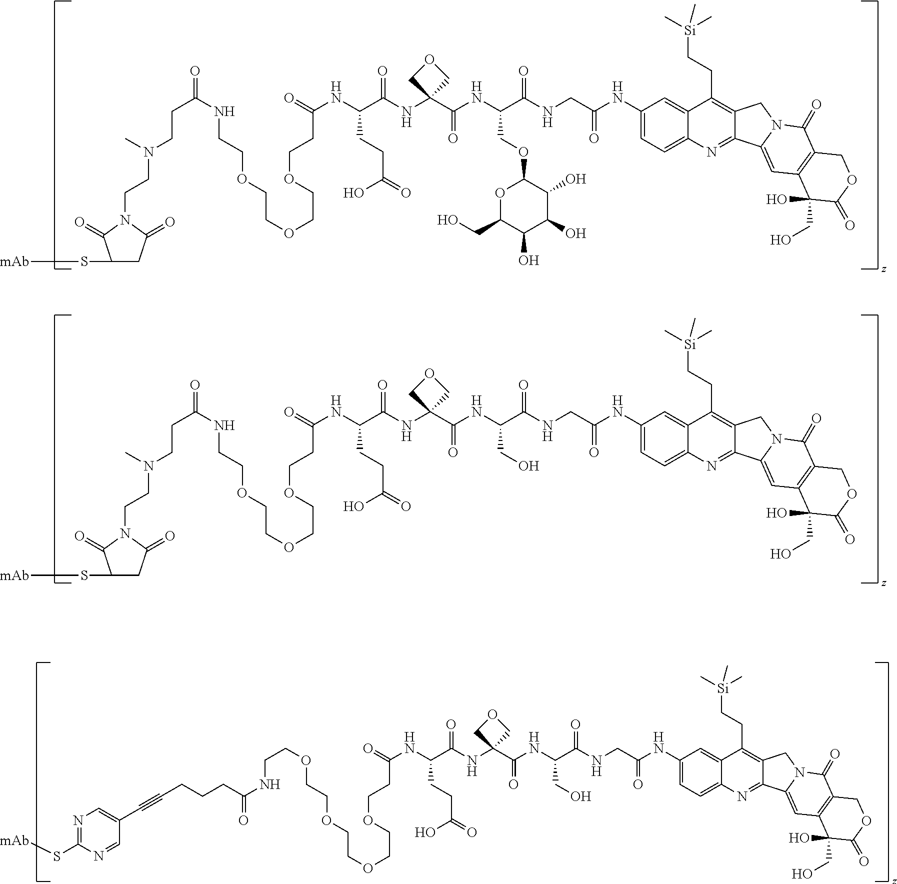 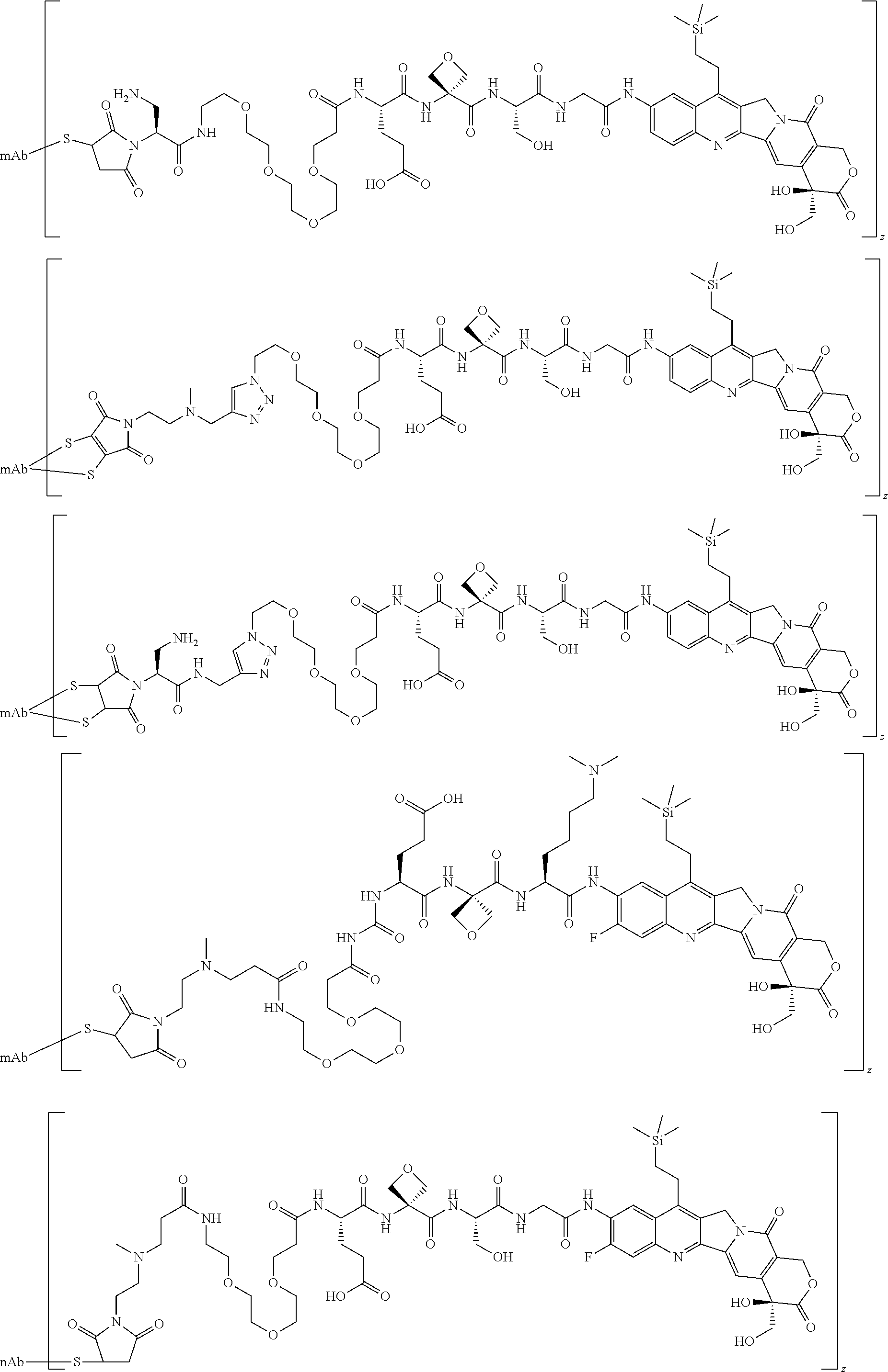 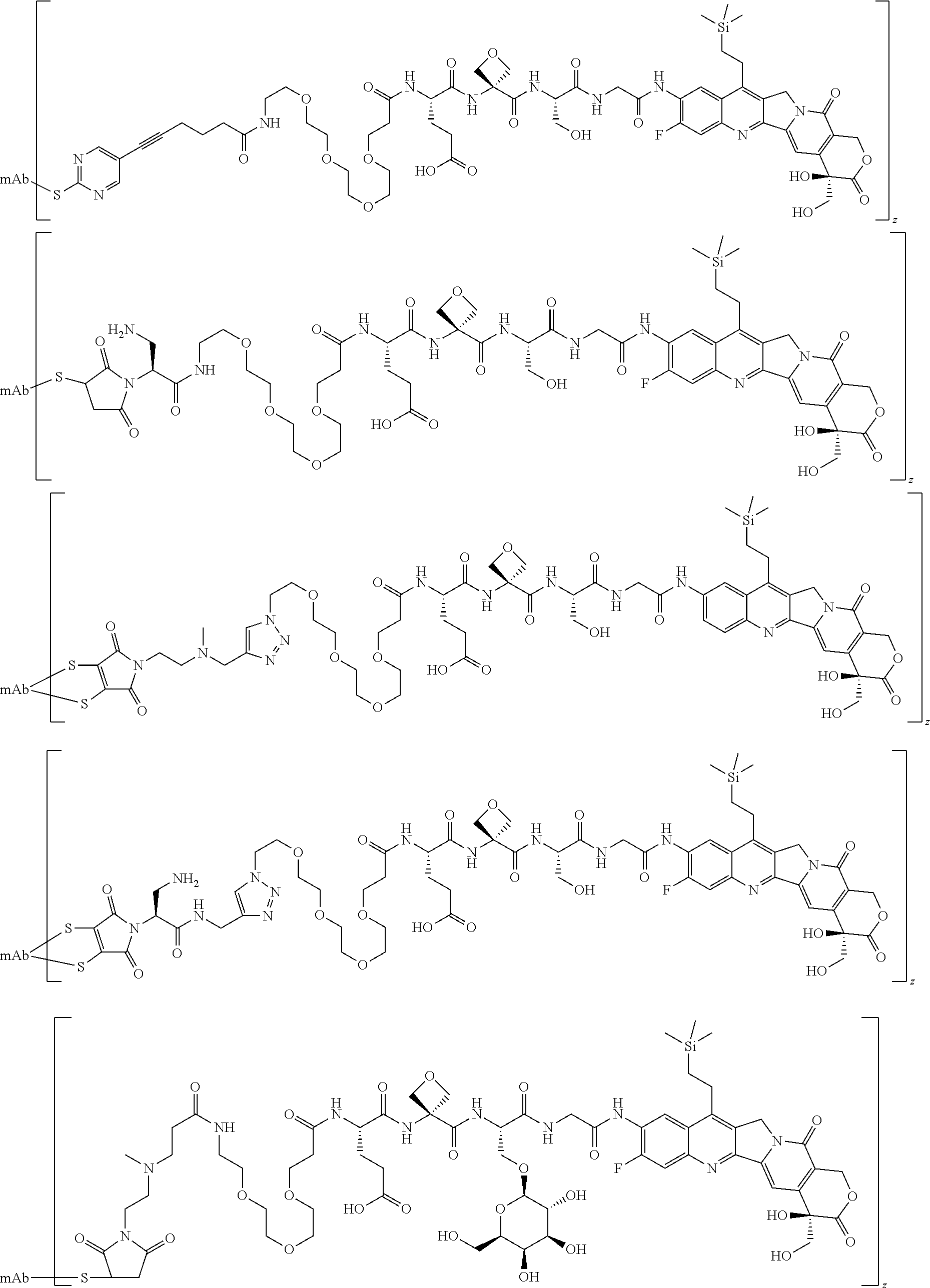 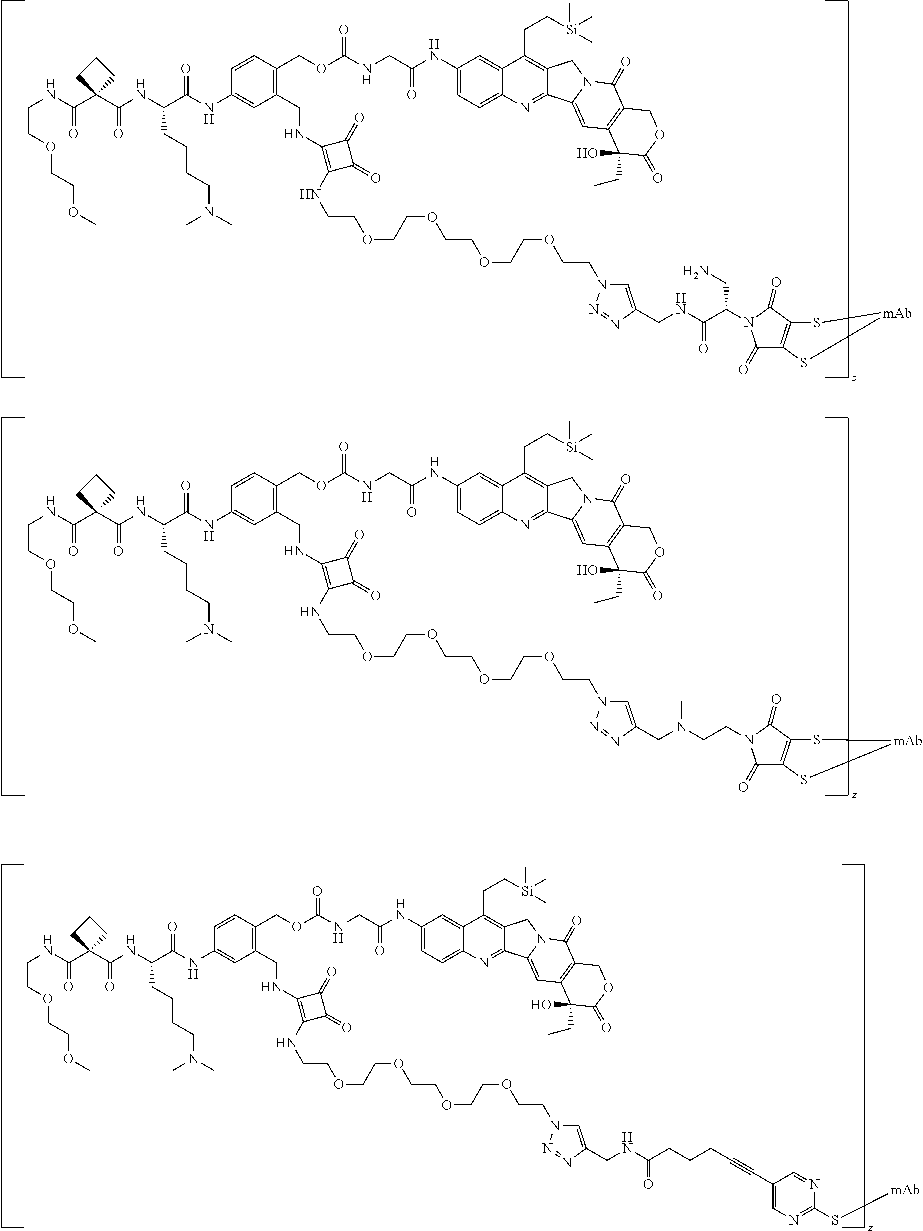 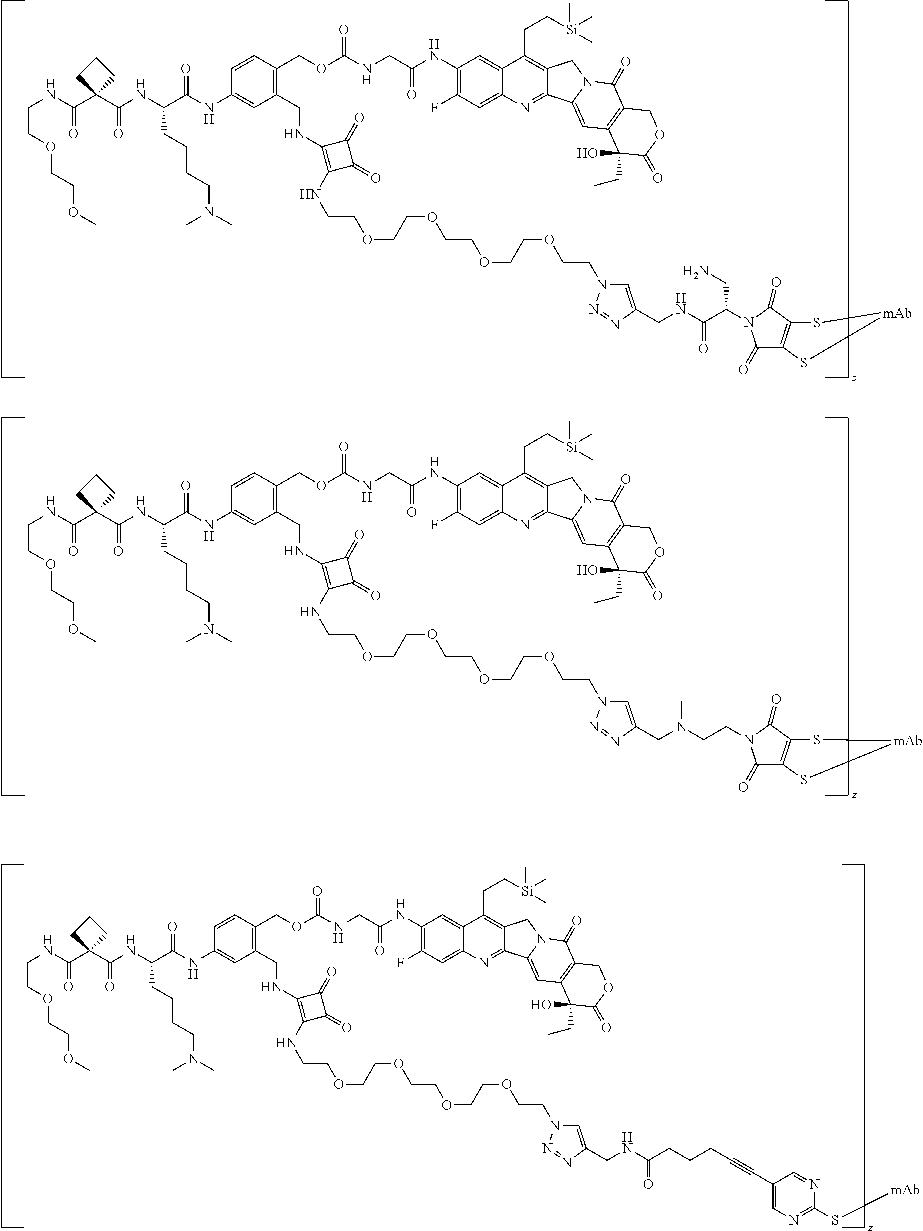 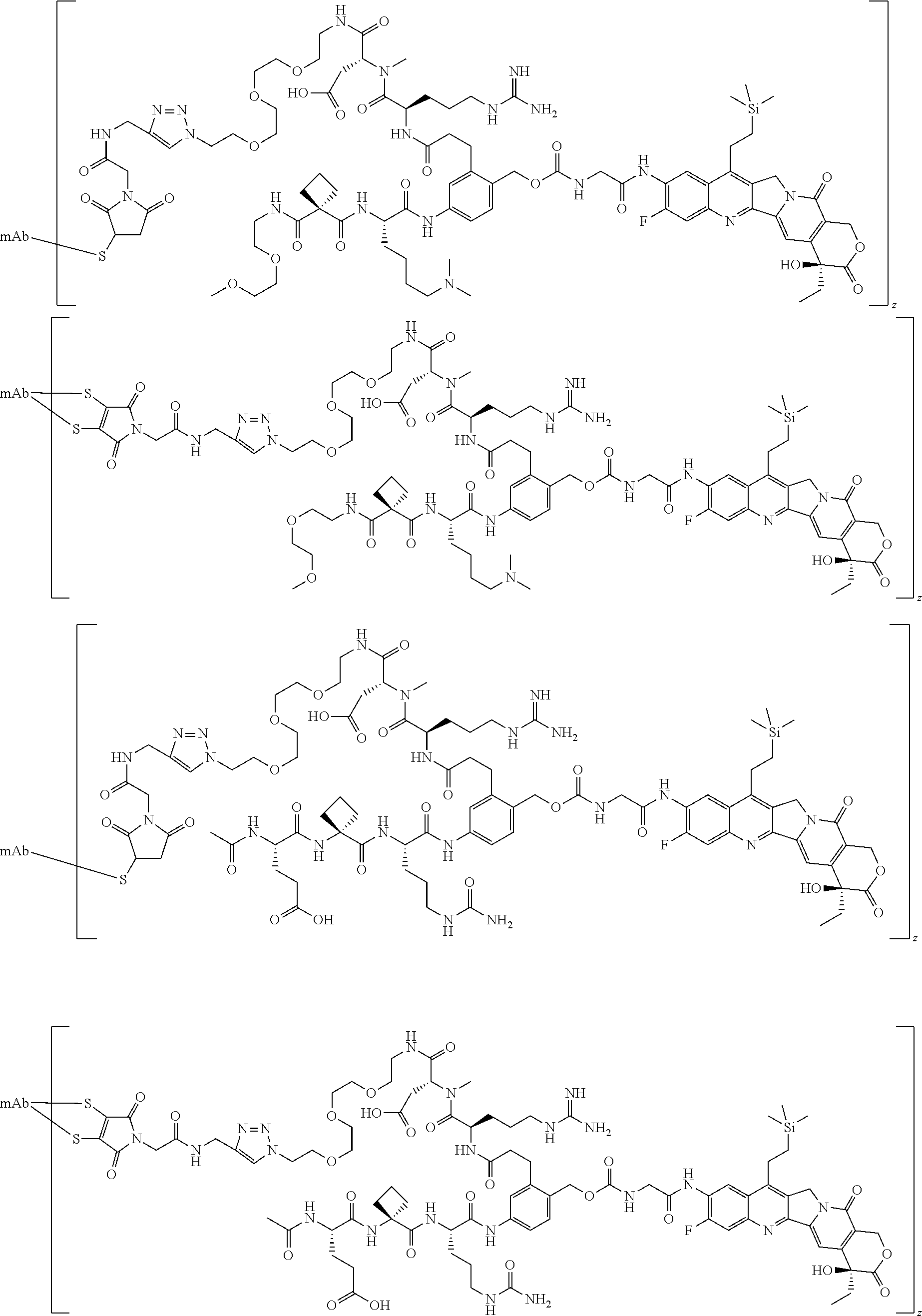 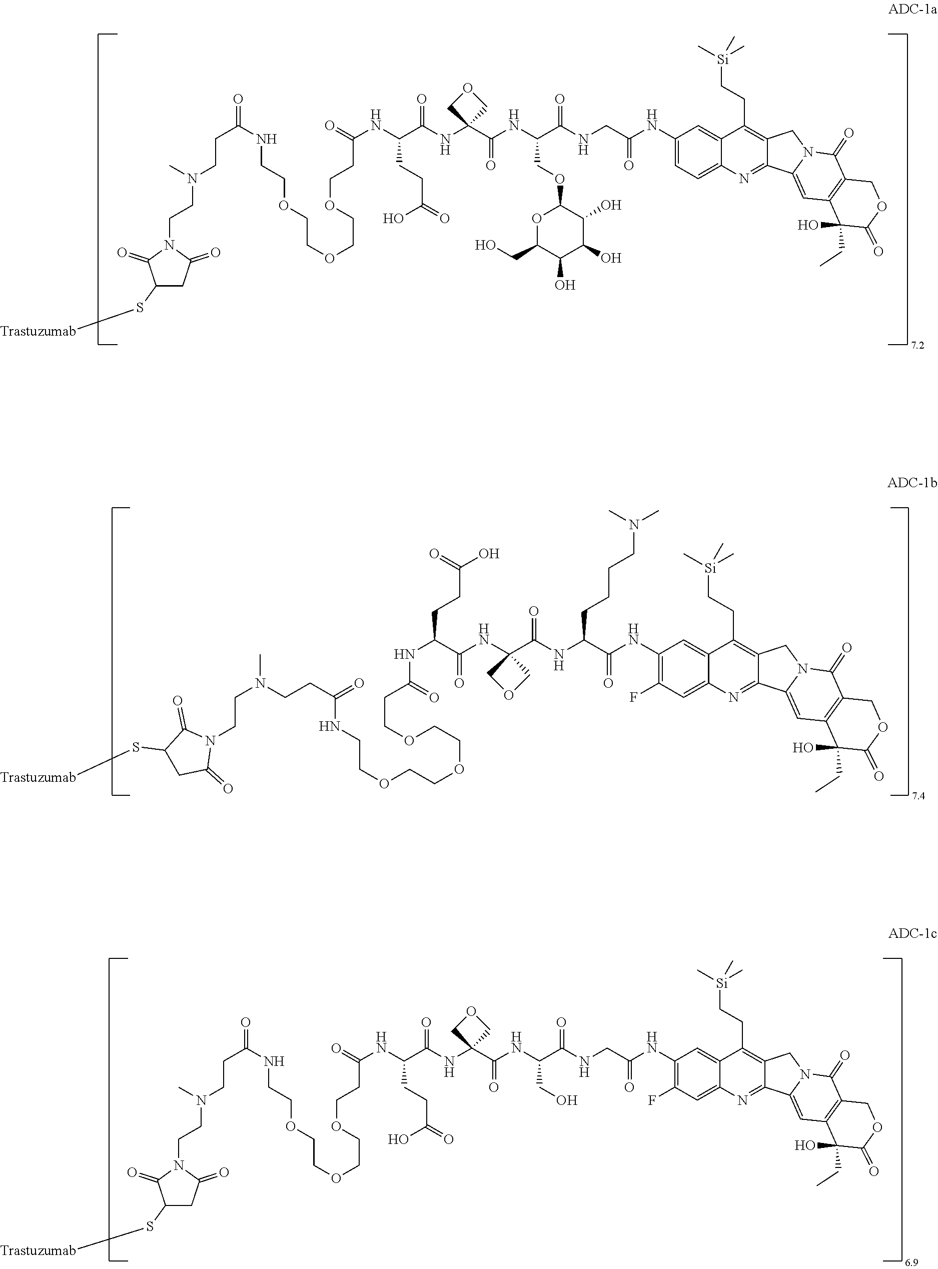
wherein, mAb is independently the Trastuzumab antibody, FG1 antibody, v30384 antibody, Gatipotuzumab antibody, DNP antibody, FG1-Tsc antibody or FG1-Tsc thiomab antibody, z is independently an integer or decimal of 1-8;
preferably, the drug conjugate of formula II is any one of the following drug conjugates or hydrolysates thereof:

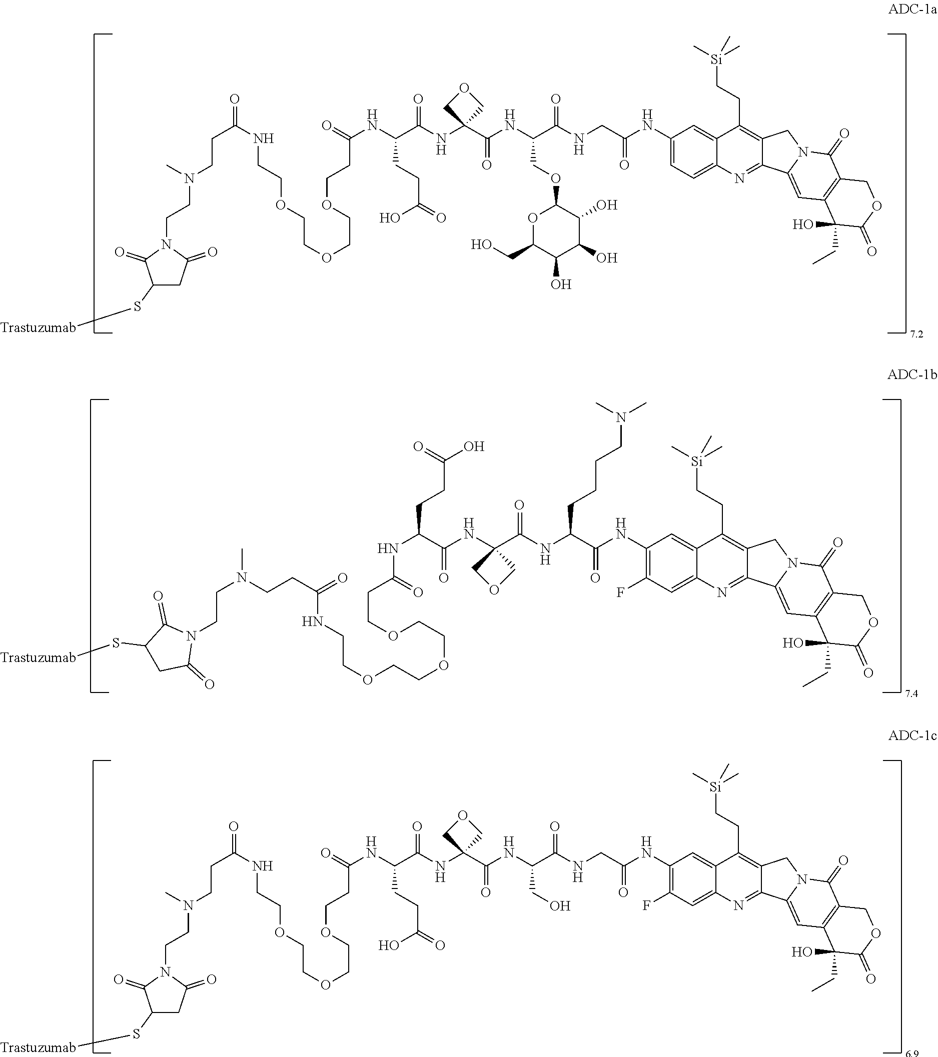

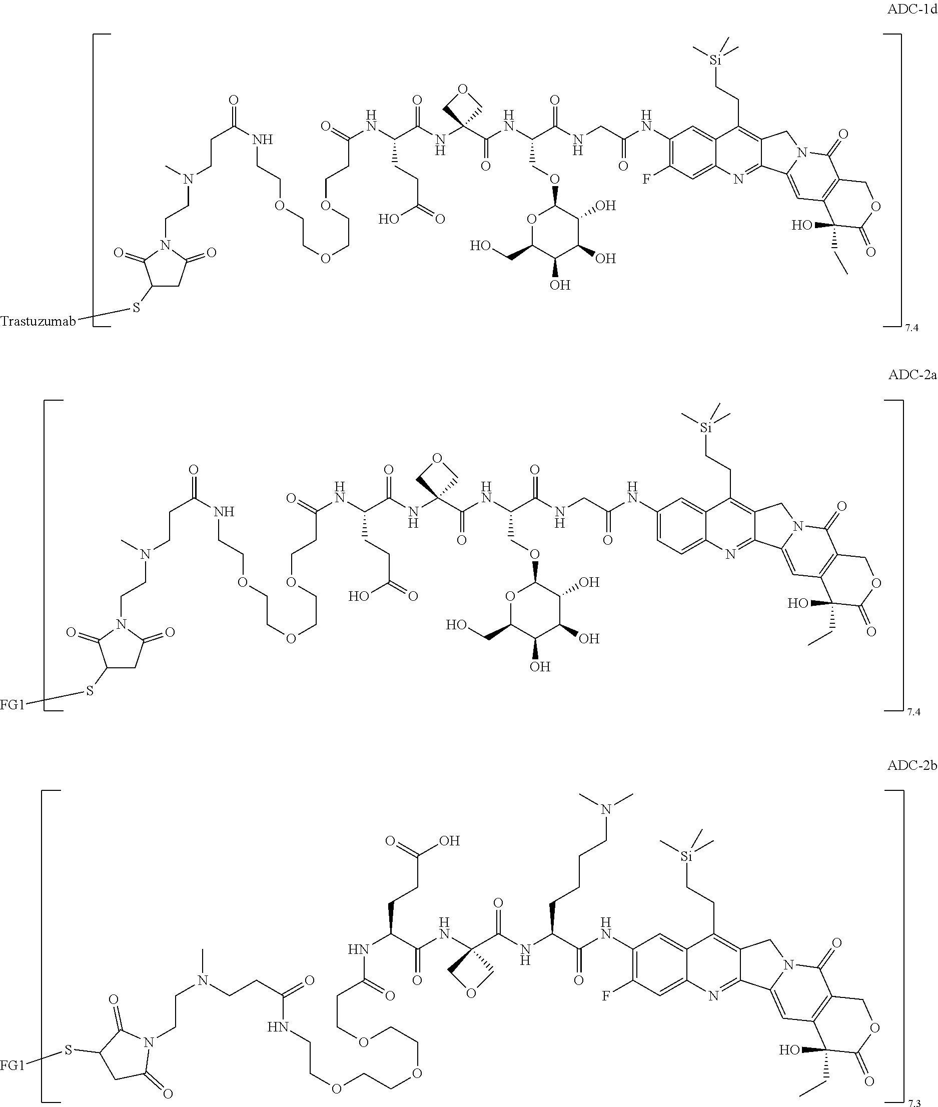

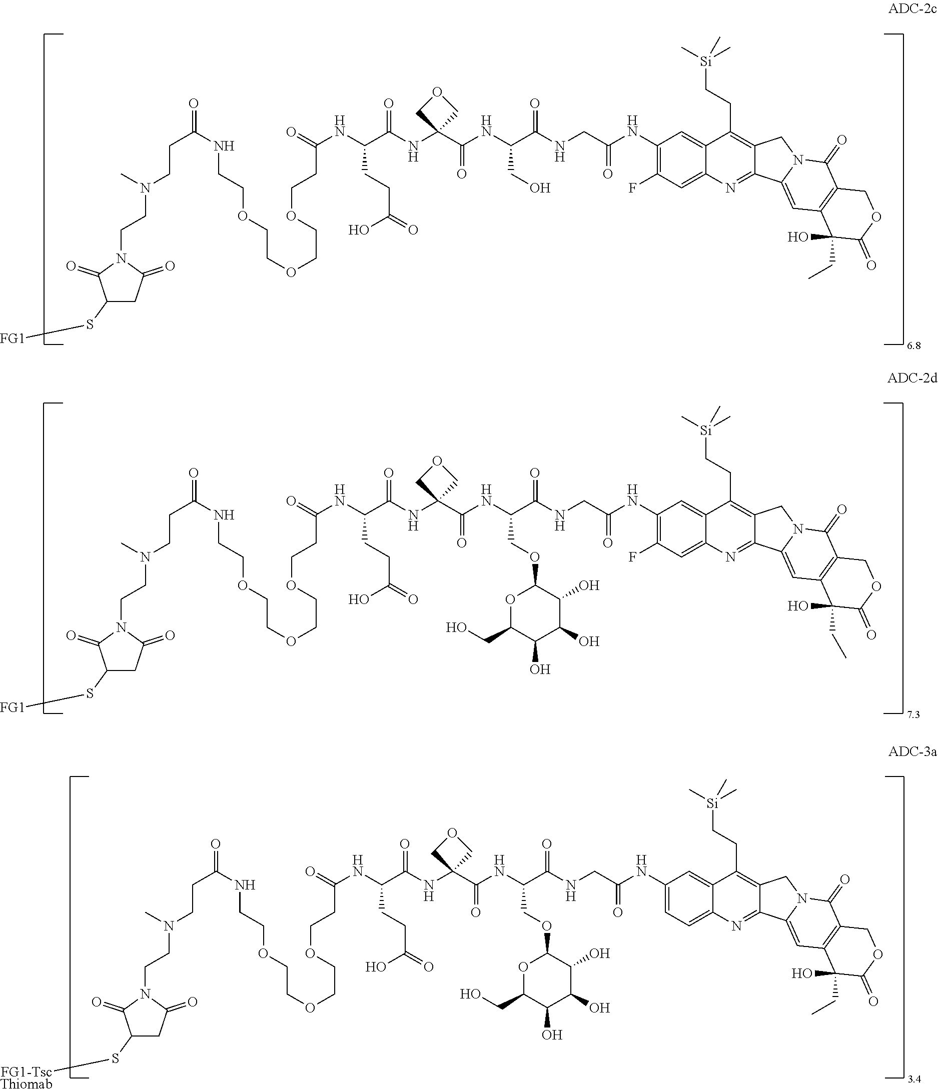

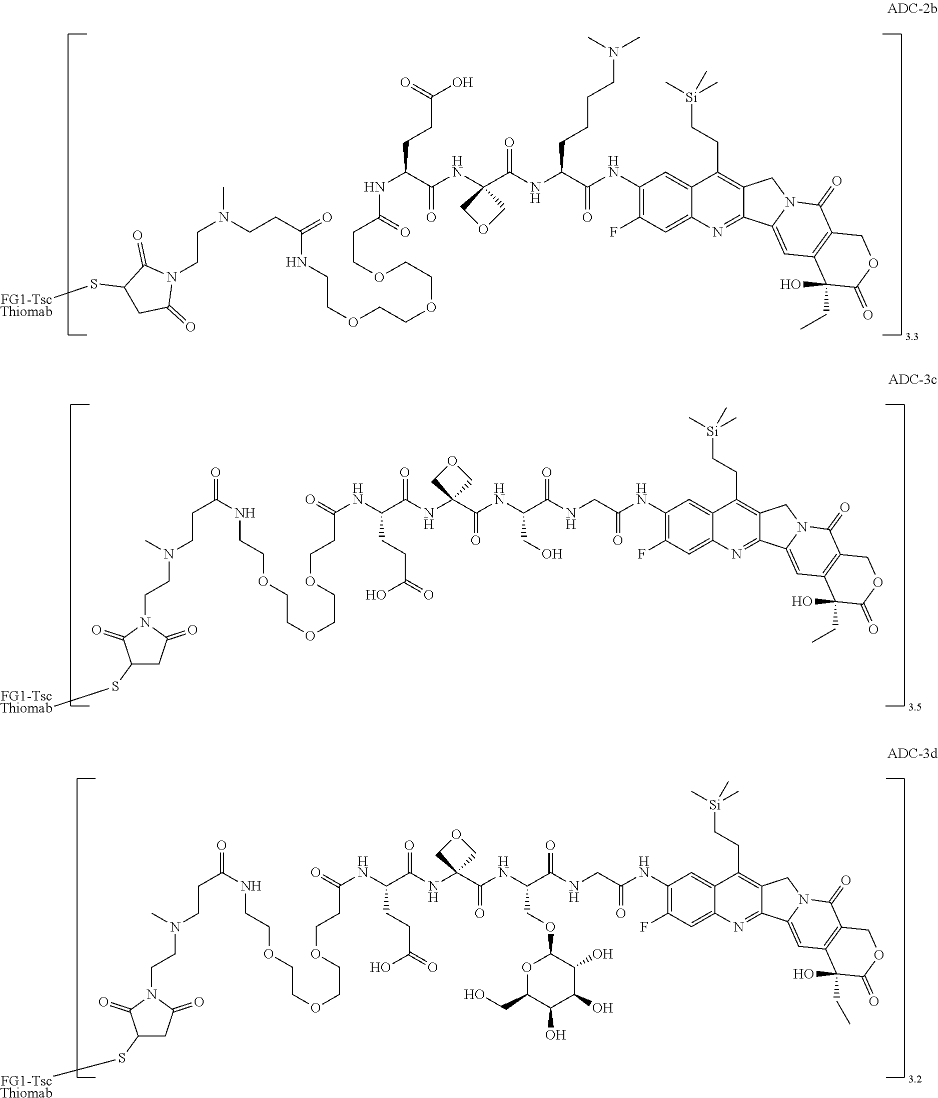

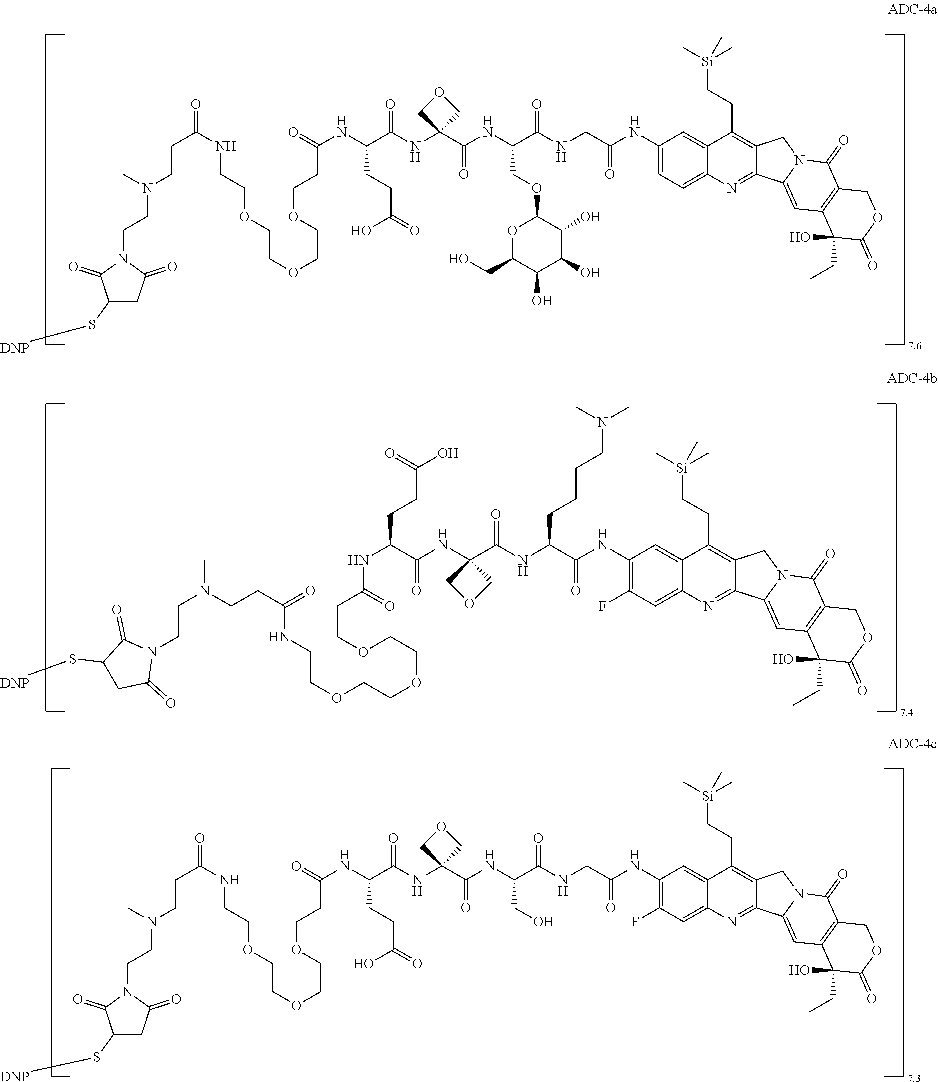

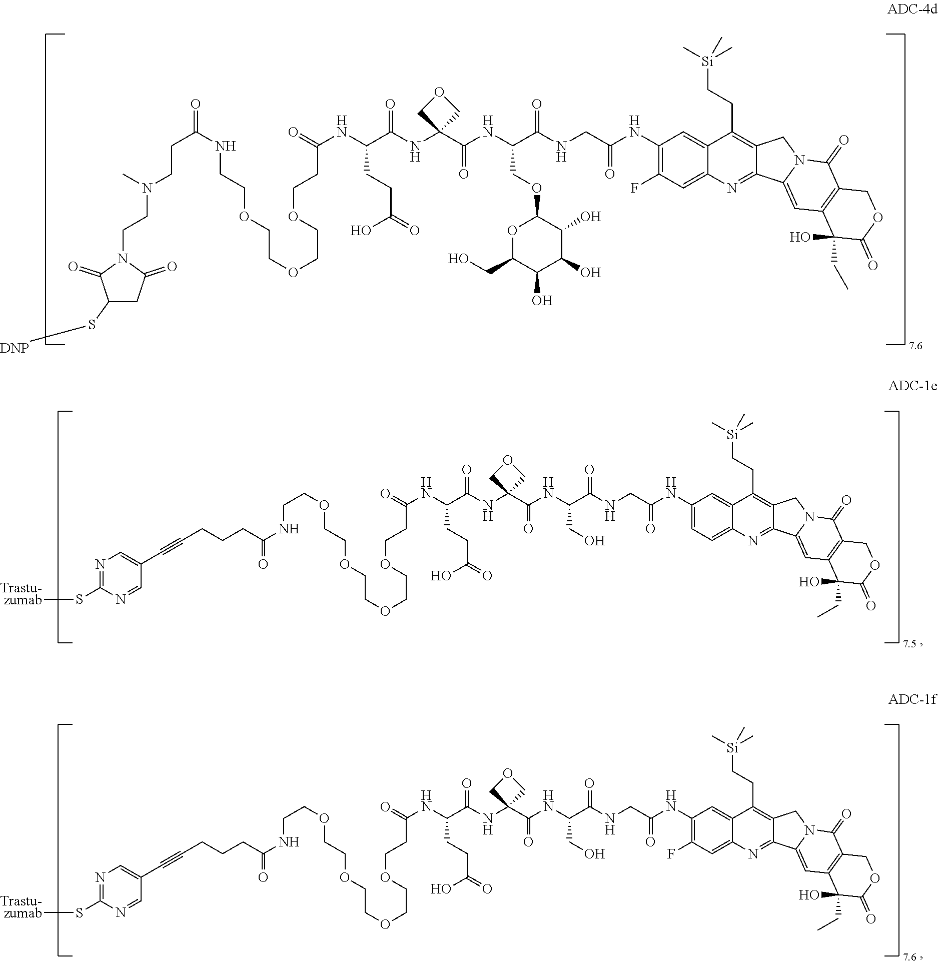

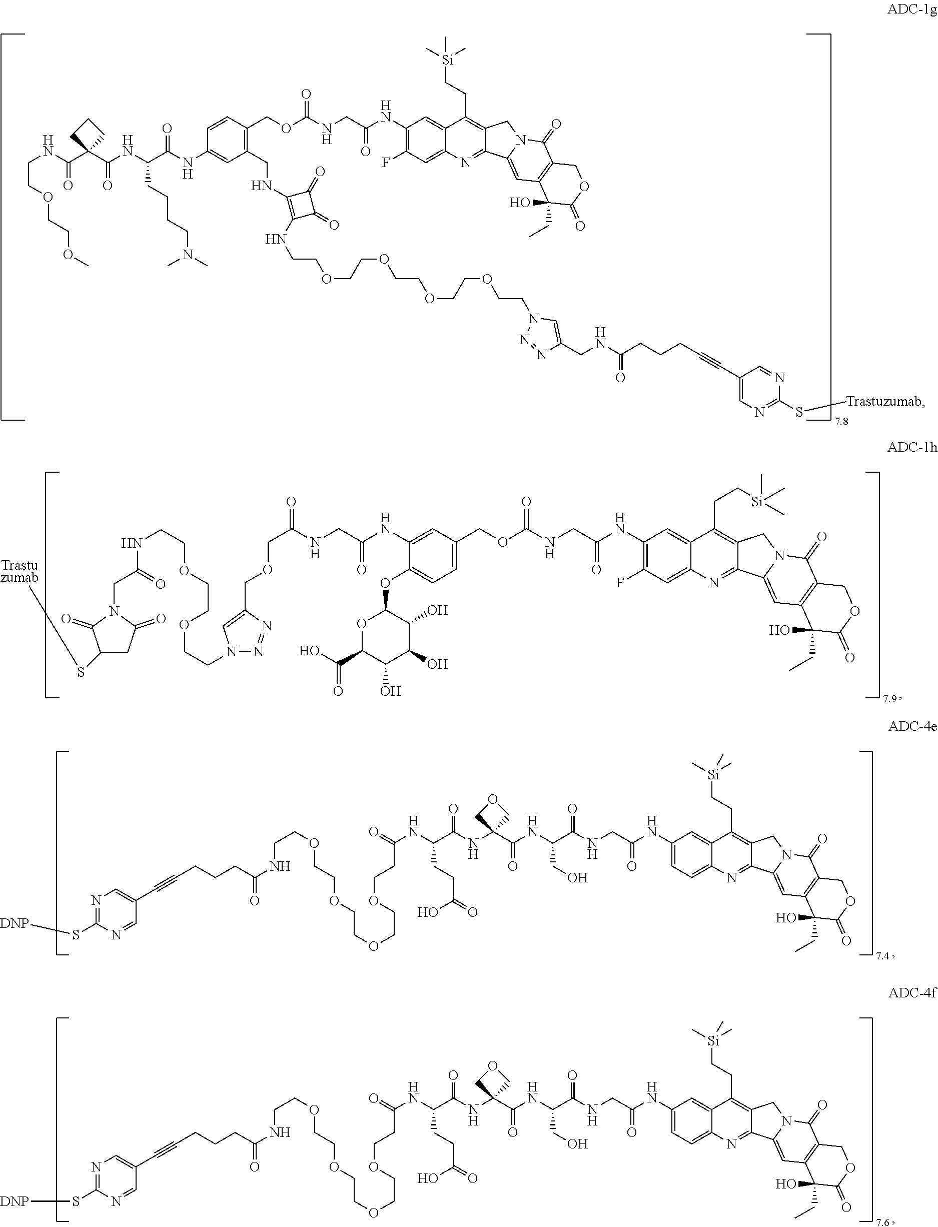

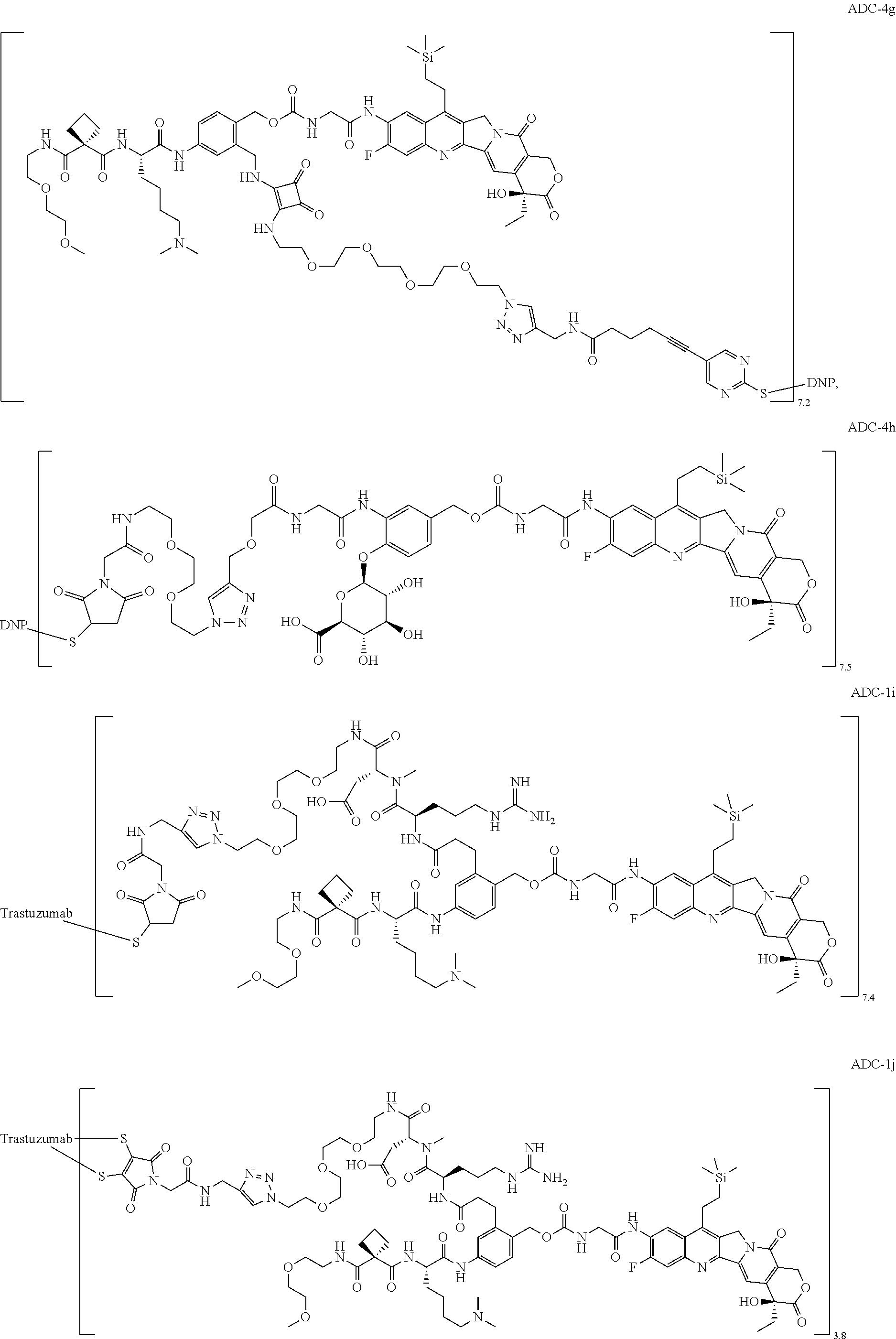

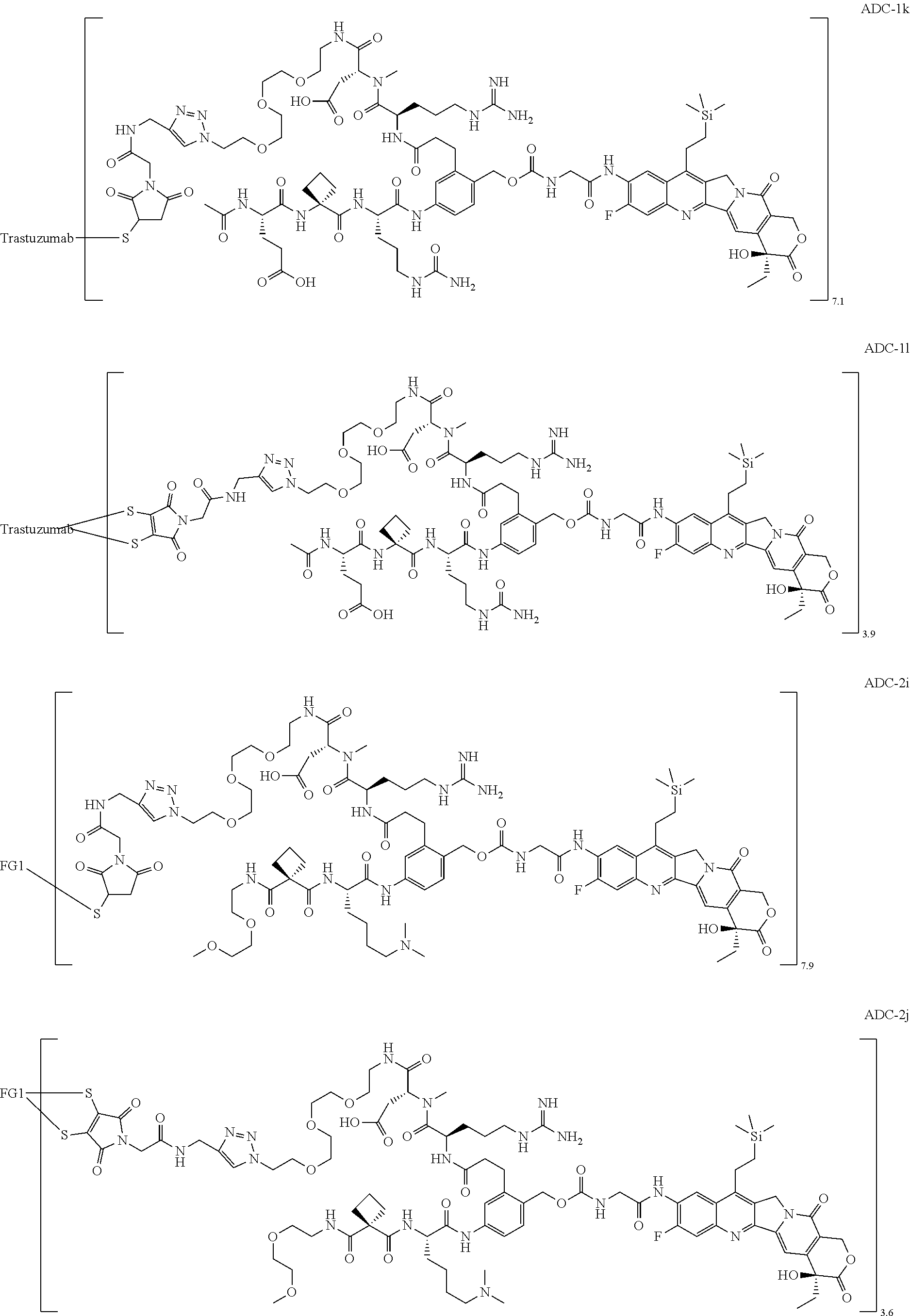

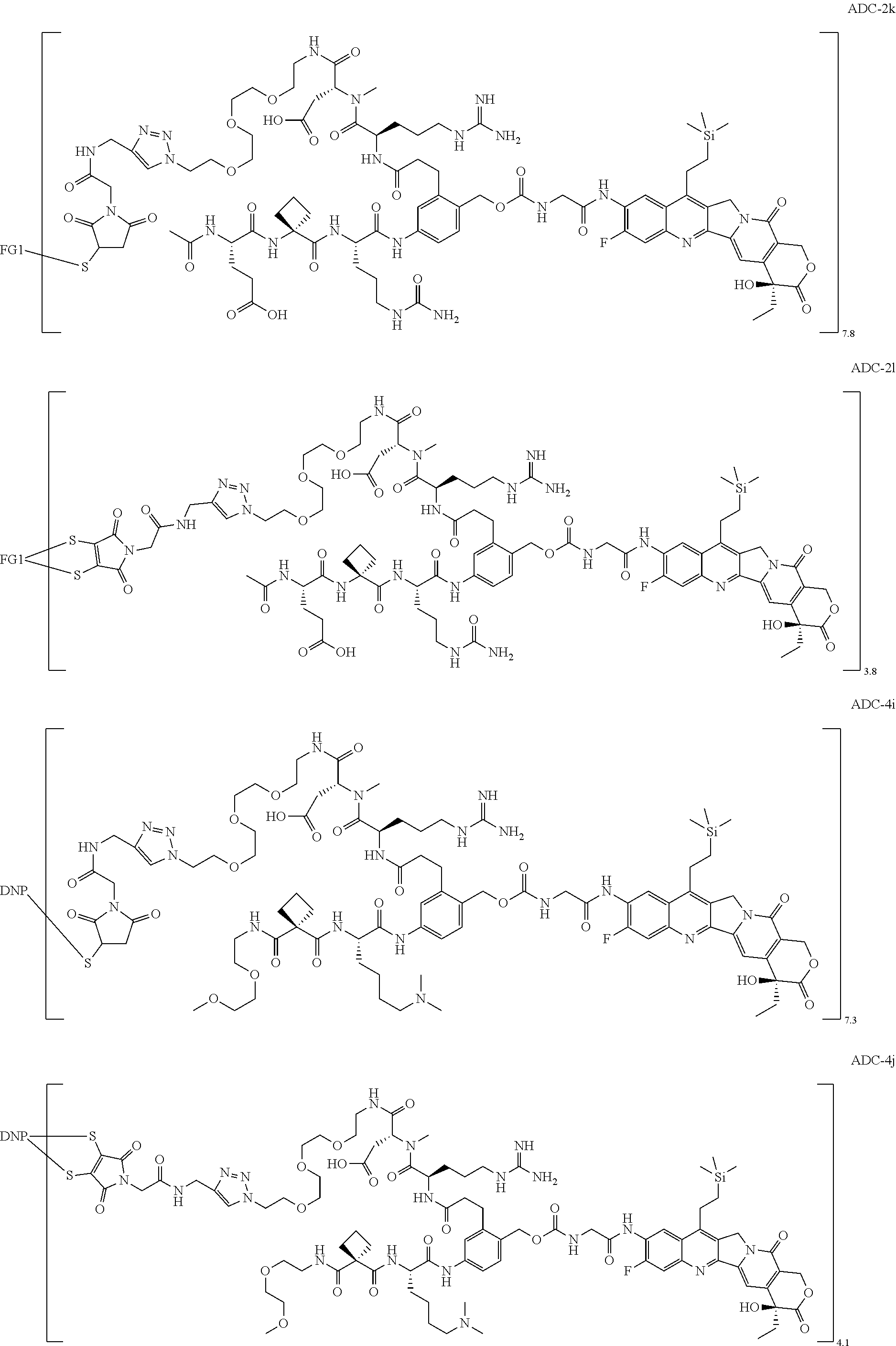

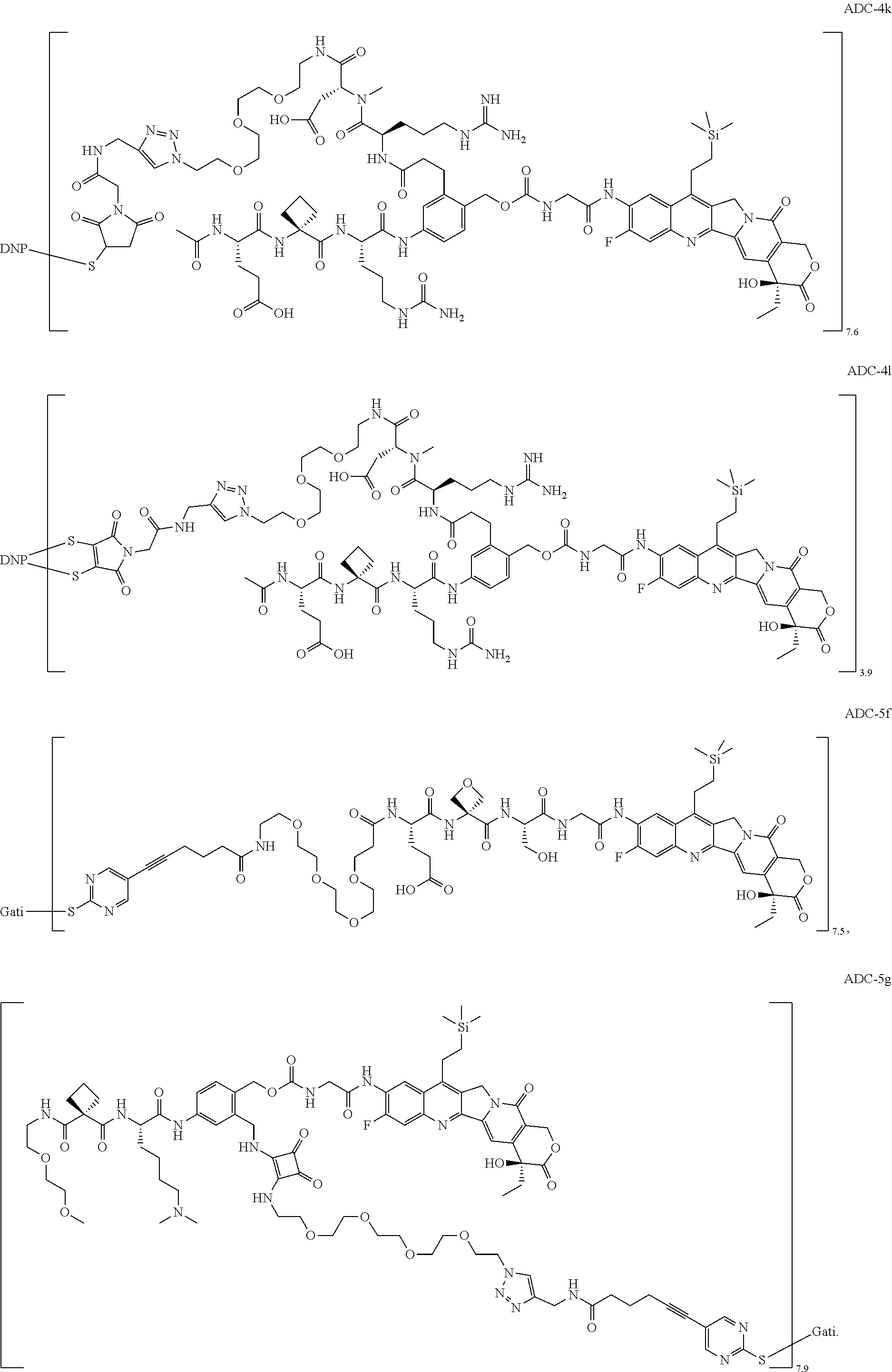

8. A compound of formula III or a pharmaceutically acceptable salt thereof: 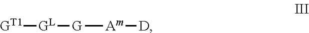
G^{T1} is a group connected to an antibody or an antigen-binding fragment thereof,
G^{L}, G, A^{m} and D are as defined in claim 1.

9. The compound of formula III or the pharmaceutically acceptable salt thereof according to claim 8, wherein
G^{T1} is 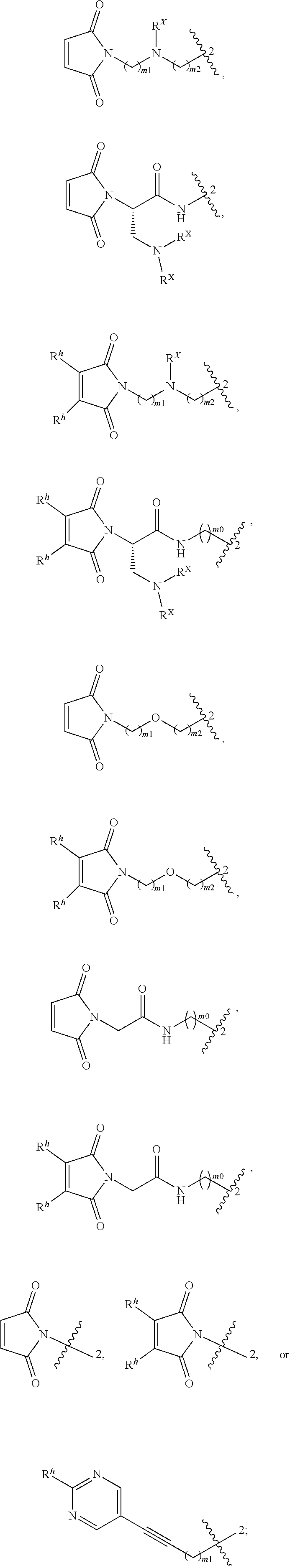
R^{h} is a leaving group, for example, R^{h} is 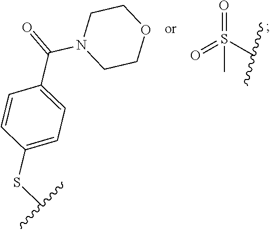
each m0 is independently 0, 1, 2, 3, 4 or 5; m1 and m2 are each independently 1, 2, 3, 4 or 5; each R^{x} is independently H or C₁₋₆ alkyl; for example, G^{T1} is

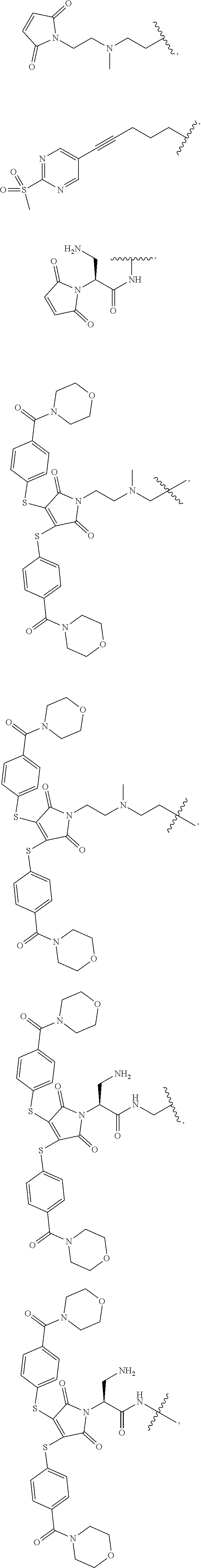

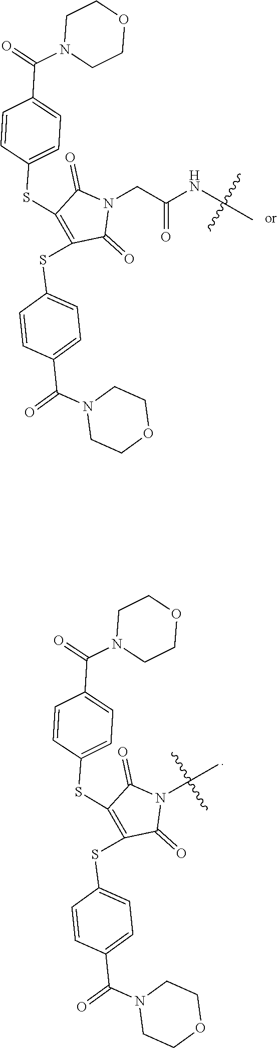 [SMILES: *NC(=O)CN1C(=O)C(Sc2ccc(C(=O)N3CCOCC3)cc2)=C(Sc2ccc(C(=O)N3CCOCC3)cc2)C1=O ; *N1C(=O)C(Sc2ccc(C(=O)N3CCOCC3)cc2)=C(Sc2ccc(C(=O)N3CCOCC3)cc2)C1=O]

10. The compound of formula III or the pharmaceutically acceptable salt thereof according to claim 8, wherein the compound of formula III is any one of the following compounds:

| Com- | |
|---|---|
| pound | |
| number | Structural formula of linker-payload |
| L3P1 | 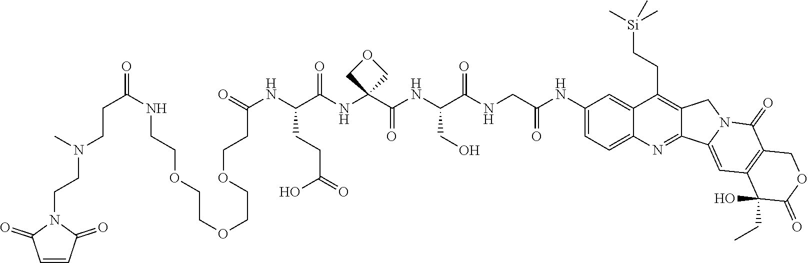 |
| L3aP1 | 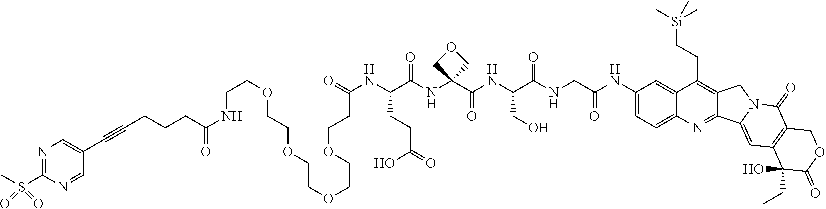 |
| L3bP1 | 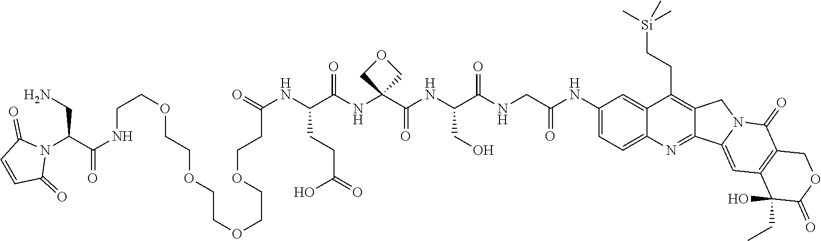 |
| L3cP1 | 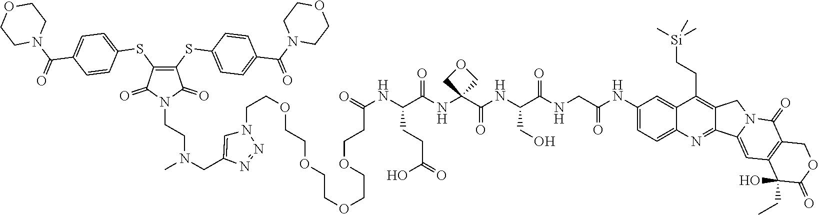 |
| L3dP1 | 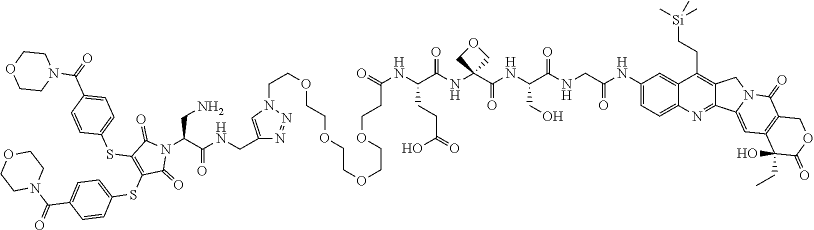 |
| L2P2 | 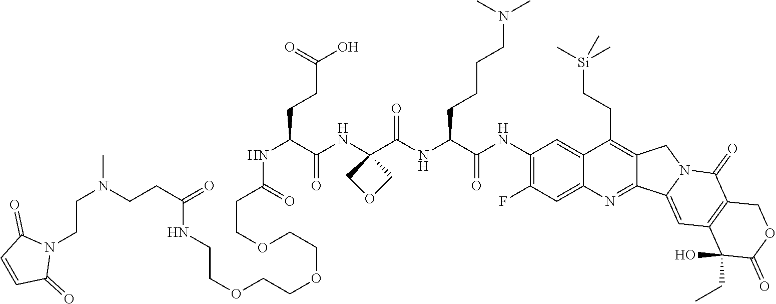 |
| L3P2 | 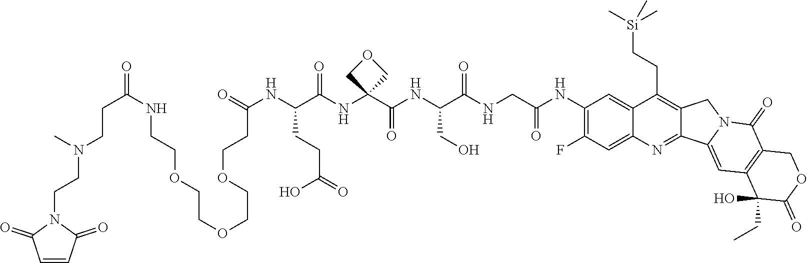 |
| L3aP2 | 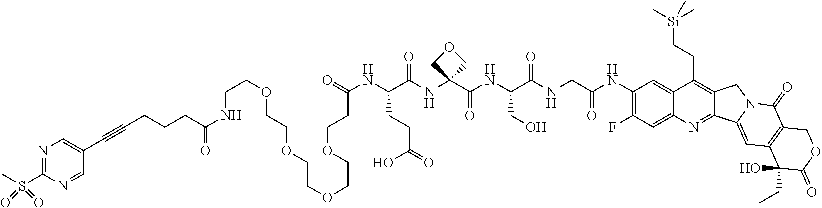 |
| L3bP2 | 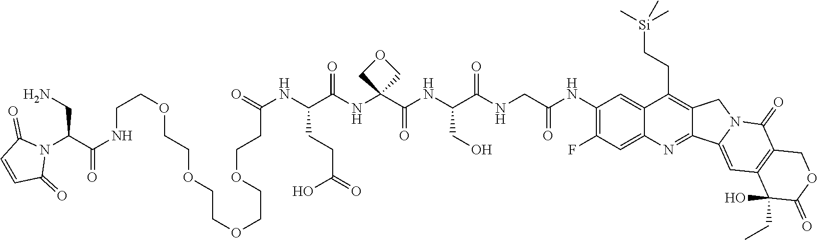 |
| L3cP2 | 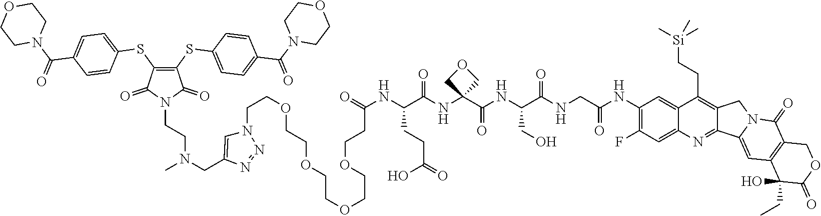 |
| L3dP2 | 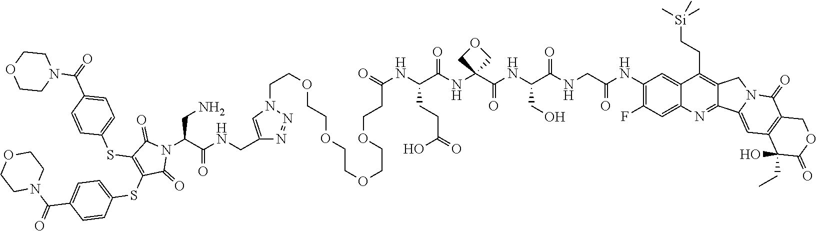 |
| L7P1 | 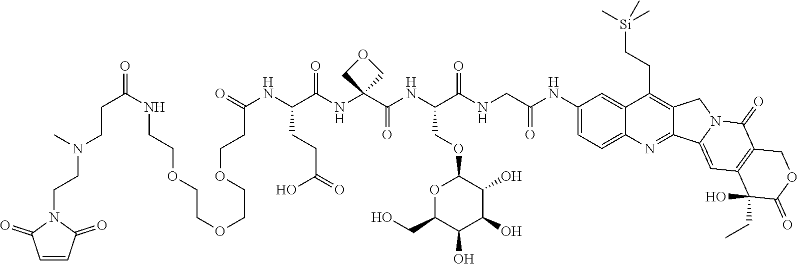 |
| L7P2 | 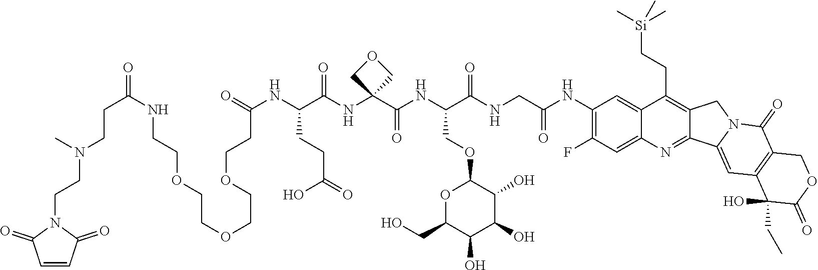 |
| L8pP1 | 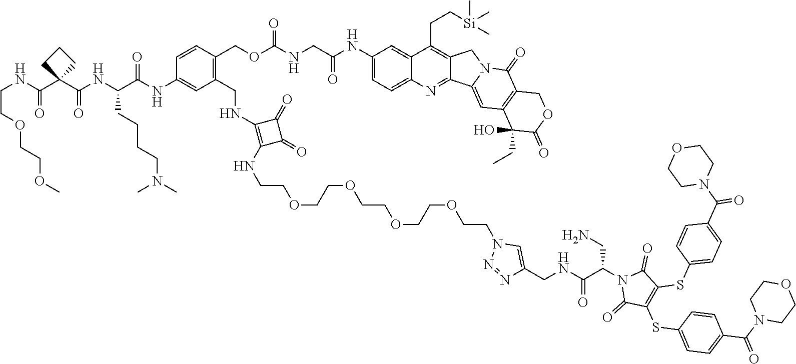 |
| L8qP1 | 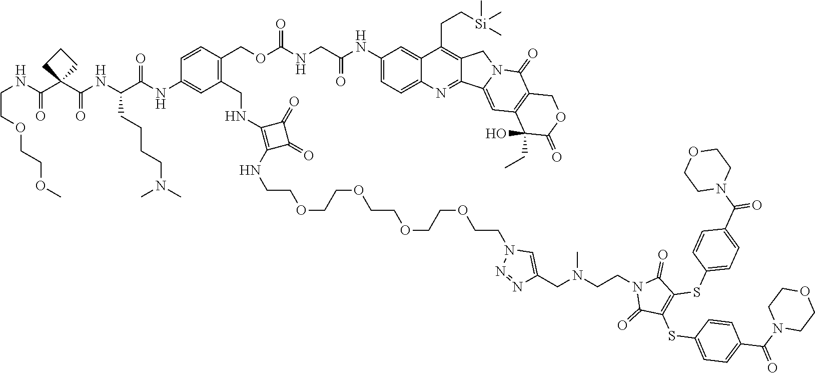 |
| L8rP1 | 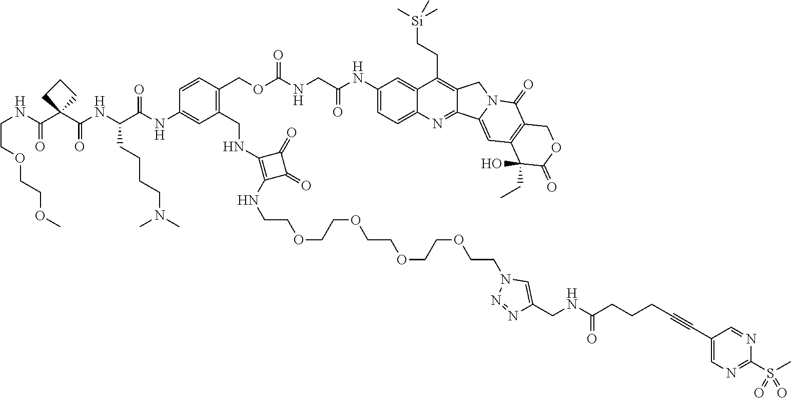 |
| L8pP2 | 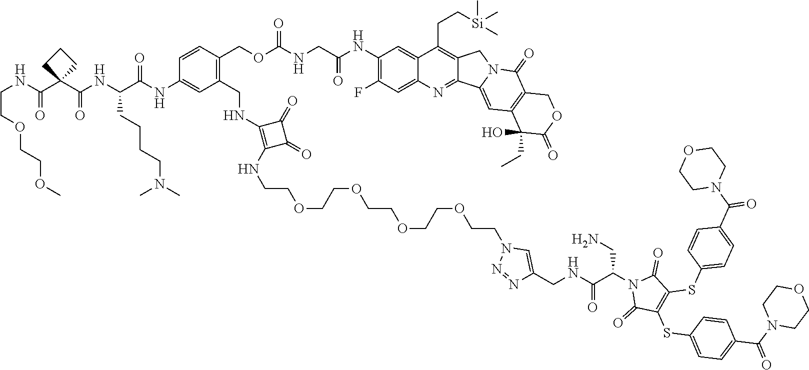 |
| L8qP2 | 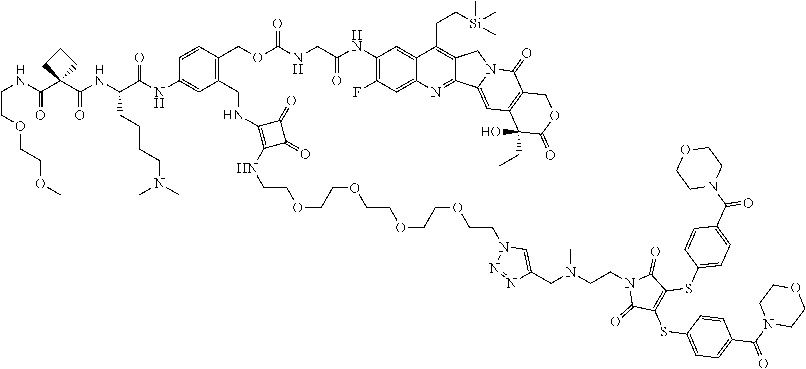 |
| L8rP2 | 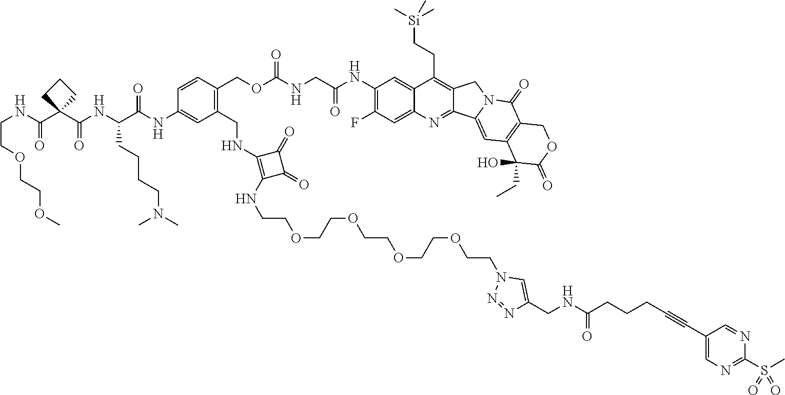 |
| L18aP2 | 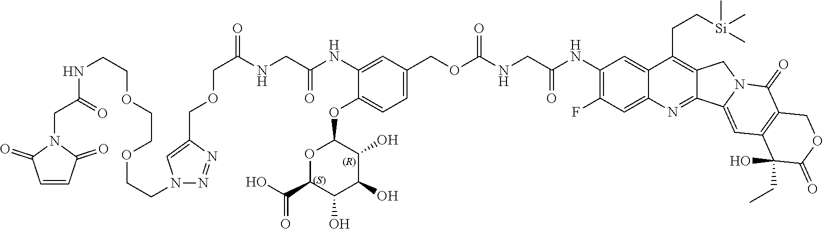 |
| L8rQP2 | 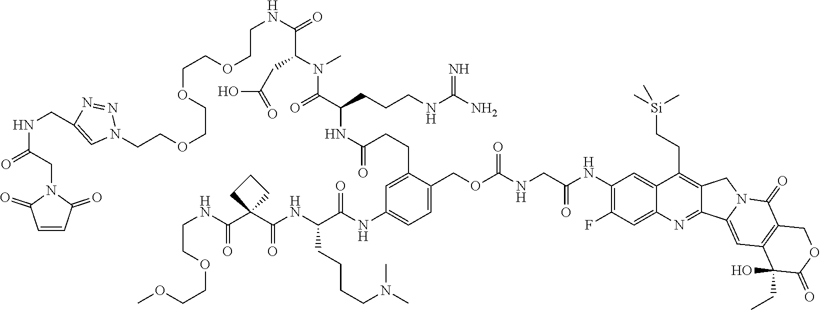 |
| L8rRP2 | 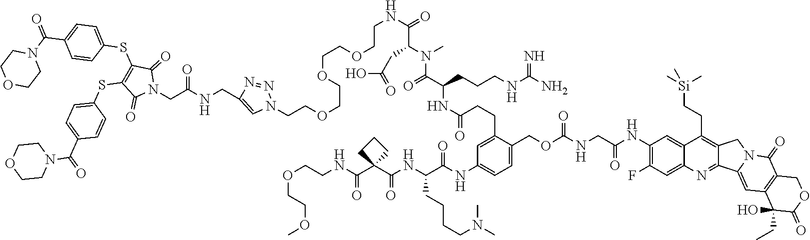 |
| L8rSP2 | 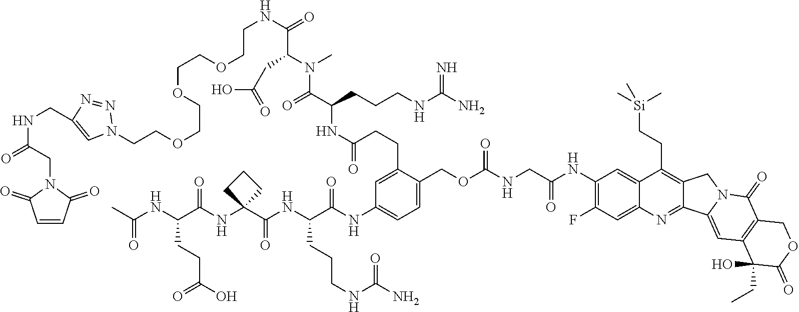 |
| L8rTP2 | 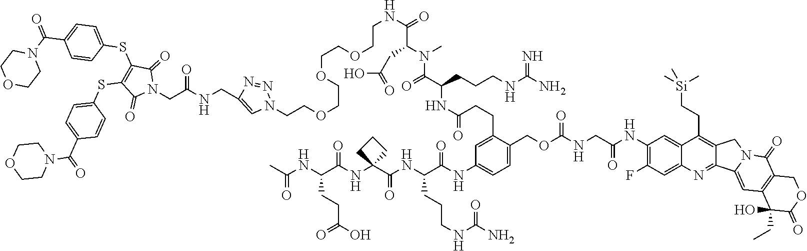 |

11. A compound of formula I or a pharmaceutically acceptable salt thereof, 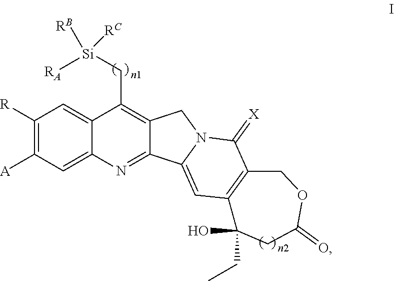
wherein n1 is 1, 2 or 3;
R is amino or hydroxyl;
A is halogen, hydroxyl, amino, C₁₋₆ alkyl, C₂₋₆ alkenyl, C₂₋₆ alkynyl, C₁₋₆ alkoxy, C₆₋₁₀ aryl, 5- to 10-membered heteroaryl, C₃₋₆ cycloalkyl or 3- to 6-membered heterocyclyl; the 5- to 10-membered heteroaryl and 3- to 6-membered heterocyclyl comprise one, two or three types of heteroatoms selected from N, O and S, and the number of the heteroatoms is independently 1, 2 or 3;
R^{A}, R^{B}, R^{C} and n2 are as defined in claim 1;
preferably, the compound of formula I is

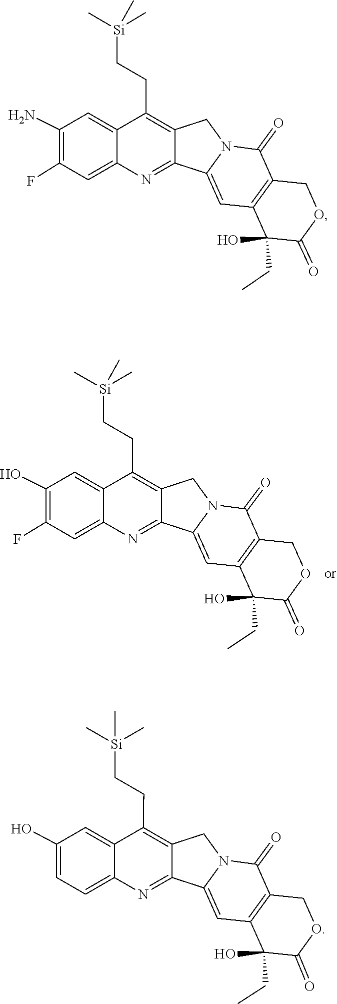 [SMILES: CC[C@@]1(O)C(=O)OCc2c1cc1n(c2=O)Cc2c-1nc1cc(F)c(N)cc1c2CC[Si](C)(C)C ; CC[C@@]1(O)C(=O)OCc2c1cc1n(c2=O)Cc2c-1nc1cc(F)c(O)cc1c2CC[Si](C)(C)C ; CC[C@@]1(O)C(=O)OCc2c1cc1n(c2=O)Cc2c-1nc1ccc(O)cc1c2CC[Si](C)(C)C]

12. A pharmaceutical composition, comprising:
(1) (a therapeutically effective amount of) the drug conjugate of formula II or the pharmaceutically acceptable salt thereof according to claim 1, and
(2) a pharmaceutical excipient.

13. A method of preventing and/or treating cancer in a subject in need thereof, comprising: administering a therapeutically effective amount of the drug conjugate of formula II or the pharmaceutically acceptable salt thereof according to claim 1 to the subject, wherein the cancer may be ovarian cancer, breast ductal carcinoma, breast cancer, pancreatic adenocarcinoma, pancreatic cancer or gastric cancer; and the pancreatic adenocarcinoma may be metastatic pancreatic adenocarcinoma.

14. A method of preventing and/or treating a disease related to an antibody target in a subject in need thereof, comprising: administering a therapeutically effective amount of the drug conjugate of formula II or the pharmaceutically acceptable salt thereof according to claim 1 to the subject;
preferably, the antibody target is one or more selected from Her2, folate receptor α, MUC1, and human IgG1, for example, the antibody target is Her2, MUC1 or both Her2 and MUC1;
more preferably, the disease related to Her2, MUC1, or Her2 and MUC1 targets is cancer, such as ovarian cancer, breast ductal carcinoma, breast cancer, pancreatic adenocarcinoma, pancreatic cancer or gastric cancer; the pancreatic adenocarcinoma may be metastatic pancreatic adenocarcinoma.


SEQUENCE LISTING (21 sequences)

SEQ ID NO: 1 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
EVQLLESGGGLVQPGGSLRLSCAVSGFSLSSYGVSWVRQAPGKGLEWIGAVNSGGSAYYADSVKGRSTISRDNSKNTVYLQMNSLRAEDTAVYYCARSGSGYPMDYLAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 4 - protein, 449 aa, unidentified
EVQLLESGGGLVQPGGSLRLSCAVSGFSLSSYGVSWVRQAPGKGLEWIGAVNSGGSAYYADSVKGRSTISRDNSKNTVYLQMNSLRAEDTAVYYCARSGSGYPMDYLAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 5 - protein, 217 aa, synthetic construct
DYQMTQSPSSVSASVGDRVTITCRASQSIGDWLAWYQQKPGKAPKLLIYEASTLASGVPSRFSGSGSGTDFTLTISSVQPEDAATYYCQQGYGRSNVDNIFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 448 aa, synthetic construct
QVQLVQSGAEVKKPGASVKVSCKASGYTFSAYWIEWVRQAPGKGLEWVGEILPGSNNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 220 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 447 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 219 aa, synthetic construct
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 450 aa, synthetic construct
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 213 aa, synthetic construct
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 705 aa, synthetic construct
QVQLVQSGAEVKKPGASVKVSCKASGYTFSAYWIEWVRQAPGKGLEWVGEILPGSNNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 13 - protein, 220 aa, synthetic construct; identical to SEQ ID NO: 7

SEQ ID NO: 14 - protein, 704 aa, synthetic construct
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 15 - protein, 219 aa, synthetic construct; identical to SEQ ID NO: 9

SEQ ID NO: 16 - protein, 707 aa, synthetic construct
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 17 - protein, 213 aa, synthetic construct; identical to SEQ ID NO: 11

SEQ ID NO: 18 - protein, 119 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARWDSSSWSFDYWGQGTLVTVSS

SEQ ID NO: 19 - protein, 108 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIKR

SEQ ID NO: 20 - protein, 705 aa, synthetic construct
QVQLVQSGAEVKKPGASVKVSCKASGYTFSAYWIEWVRQAPGKGLEWVGEILPGSNNSRYNEKFKGRVTVTRDTSTNTAYMELSSLRSEDTAVYYCARSYDFAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGKGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 21 - protein, 220 aa, synthetic construct
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC